(19) United States
(12) Patent Application Publication
(10) Pub. No.: US 2025/0136589 A1
(43) Pub. Date: May 1, 2025

(54) HETEROBIFUNCTIONAL COMPOUNDS AS HPK1 DEGRADERS

(71) Applicants: Icahn School of Medicine at Mount Sinai, New York, NY (US); Cullinan Oncology, Inc., Cambridge, MA (US)

(72) Inventors: Jian Jin, New York, NY (US); Steven Burakoff, New York, NY (US); H. Umit Kaniskan, New York, NY (US); Sansana Sawasdikosol, New York, NY (US); David Witter, Killington, VT (US)

(21) Appl. No.: 18/711,706

(22) PCT Filed: Nov. 23, 2022
(86) PCT No.: PCT/US2022/050929

§ 371 Date: May 20, 2024

Related U.S. Application Data
(60) Provisional application No. 63/264,506, filed on Nov. 23, 2021.

(51) Int. Cl. C07D 413/14; A61K 31/4545; A61K 31/496; A61K 31/506; C07D 471/04; C07D 471/10; C07D 519/00

(52) U.S. Cl. CPC C07D 413/14; A61K 31/4545; A61K 31/496; A61K 31/506; C07D 471/04; C07D 471/10; C07D 519/00

(57) ABSTRACT

Disclosed are Hematopoietic Progenitor Kinase 1 (HPK1) degradation/disruption compounds including a HPK1 ligand, a degradation/disruption tag and a linker, and methods for use of such compounds in the treatment of HPK1-mediated diseases.

### TECHNICAL FIELD

This disclosure relates to bivalent compounds (e.g., heterobifunctional compounds) which degrade and/or disrupt Hematopoietic Progenitor Kinase 1 (HPK1), compositions comprising one or more of the bivalent compounds, and methods of use thereof for the treatment of HPK1-mediated diseases in a subject in need thereof The disclosure also relates to methods for designing such bivalent compounds.

### BACKGROUND OF THE INVENTION

Unlike traditional enzyme inhibitors, which only inhibit the catalytic activity of the target enzyme, heterobifunctional compounds, also known as proteolysis-targeted chimeras (PROTACs), bind and induce degradation of the enzyme, thus eliminating potential scaffolding functions of the protein, in addition to inhibiting its enzymatic activity (Buckley and Crews, 2014). The HPK1 degraders disclosed herein offer a novel mechanism for treating HPK1-mediated diseases. Additionally, the ability of the degraders to target HPK1 for degradation, as opposed to inhibiting the catalytic activity of HPK1, is expected to overcome resistance, regardless of whether due to the drugs used in prior treatments or whether acquired resistance was caused by gene mutation, amplification or otherwise.

The immune system constantly surveil the host for the emergence of nascent cancer cells and eliminate them before they become tumors (Burnet, 1970). While several lines of evidence suggested that our immune system could accomplish such task (Corthay, 2014), the most direct support of such concept comes from the development of immuno-oncological drugs that target the inhibitory molecules that hinder the anti-tumor immunity effort, allowing immune system to vigorously engage and eliminate previously difficult to treat cancers. (Ribas and Wolchok, 2018) The success of the immune checkpoint inhibitor approach provides the roadmap as to how the exhausted immune systems could be provoked to re-engage the cancer cells. This theoretical framework spurs the search for novel immune checkpoint receptors that could serve as novel immune checkpoint targets.

Hematopoietic Progenitor Kinase 1 (HPK1, also known as MAP4K1), an intracellular negative regulator of the T cell antigen receptor (TCR) signal transduction pathways is a member of the KHS subfamily of Ste20 serine/threonine kinases (Hu et al., 1996; Kiefer et al., 1996). HPK1 is a multimodular-domain protein comprising proline-rich motifs and a C-terminal citron homology domain, in addition to its kinase domain. These additional domains suggest that HPK1 could perform functions other than its kinase function. HPK1 transcripts are detected in all embryonic tissues examined, but its expression profile shifts to a hematopoietic cell-restricted pattern post-partum at neonatal day 1 (Kiefer et al., 1996), leading to the speculation that HPK1 may perform a specialized function in hematopoietic cells. This cytosolic Ste20 kinase is recruited to the TCR complex (Ling et al., 2001) and its kinase activity is induced upon the engagement of the TCR (Liou et al., 2000). Overexpression of HPK1 suppresses TCR-induced activation of AP-1-dependent gene transcription in a kinase dependent manner, suggesting that the kinase activity of HPK1 is required to inhibit the Erk MAPK pathway (Liou et al., 2000). This blockage of the Erk MAPK pathway is thought to be the inhibitory mechanism that negatively regulates TCR-induced IL-2 gene transcription.

### SUMMARY OF THE INVENTION

The present disclosure relates generally to bivalent compounds (e.g., bi-functional compounds) which degrade and/or disrupt HPK1 and to methods for the treatment of HPK1-mediated diseases (i.e., a disease which depends on HPK1; overexpresses HPK1; depends on HPK1 activity; or includes elevated levels of HPK1 activity relative to a wild-type tissue of the same species and tissue type). It is important to note, because the HPK1 degraders/disruptors have dual functions (enzyme inhibition plus protein degradation/disruption), the bivalent compounds of the present disclosure can be significantly more effective therapeutic agents than currently available HPK1 inhibitors, which inhibit the enzymatic activity of HPK1, but do not affect HPK1 protein levels. The present disclosure further provides methods for identifying HPK1 degraders/disruptors as described herein.

More specifically, the present disclosure provides a bivalent compound including a HPK1 ligand conjugated to a degradation/disruption tag.

In some aspects, the HPK1 degraders/disruptors have the form “PI-linker-EL”, as shown below:

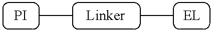

wherein PI (protein of interest) comprises an HPK1 ligand (e.g., an HPK1 inhibitor) and EL (E3 ligase) comprises a degradation/disruption tag (e.g., E3 ligase ligand). Exemplary HPK1 ligands (PI), exemplary degradation/disruption tags (EL), and exemplary linkers (Linker) are illustrated below:

### HPK1 Ligands

In an embodiment, (HPK1) ligands include a moiety according to FORMULA 1:

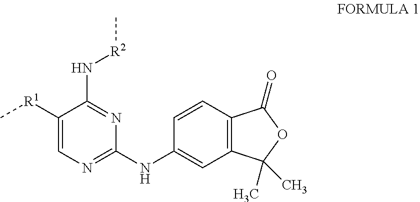

wherein
the “Linker” moiety of the bivalent compound is attached independently to R¹ or R² (indicated by dotted line);
When the “Linker” moiety of the bivalent compound is attached to R²;
R¹ is, at each occurence, independently selected from hydrogen, halogen, oxo, CN, NO₂, OR⁴, SR⁴, NR⁴R⁵, C(O)R⁴, C(O)OR⁴, C(O)NR⁴R⁵, S(O)R⁴, S(O)₂R⁴, S(O)₂NR⁴R⁵, NR⁶C(O)OR⁴, NR⁶C(O)R⁴, NR⁶C(O)NR⁴R⁵, NR⁶S(O)R⁴, NR⁶S(O)₂R⁴, NR⁶S(O)₂NR⁴R⁵, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl; wherein

R⁴, R⁵, and R⁶ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl; or

R⁴ and R⁵, R⁴ and R⁶, R⁵ and R⁶ together with the atom to which they are connected form an optionally substituted 3-20 membered cycloalkyl or heterocyclyl ring; and pharmaceutically acceptable salts thereof.

When the “Linker” moiety of the bivalent compound is attached to R¹;
R¹ is, at each occurence, independently selected from OR⁴, SR⁴, NR⁴R⁵, C(O)R⁴, C(O)OR⁴, C(O)NR⁴R⁵, S(O)R⁴, S(O)₂R⁴, S(O)₂NR⁴R⁵, NR⁶C(O)OR⁴, NR⁶C(O)R⁴, NR⁶C(O)NR⁴R⁵, NR⁶S(O)R⁴, NR⁶S(O)₂R⁴, NR⁶S(O)₂NR⁴R⁵, C₁-C₈ alkylene, optionally substituted C₁-C₈ alkylene, optionally substituted C₂-C₈ alkenylene, optionally substituted C₂-C₈ alkynylene, optionally substituted C₁-C₈ alkoxylene, optionally substituted C₁-C₈alkoxyC₁-C₈alkylene, optionally substituted C₁-C₈alkylaminoC₁-C₈alkylene, optionally substituted 3-8 membered cycloalkylene, optionally substituted C₃-C₈ cycloalkoxylene, optionally substituted 3-8 membered heterocyclylene, optionally substituted arylene, and optionally substituted heteroarylene; wherein

R⁴ is null, or a bivalent moiety selected from optionally substituted C₁-C₈ alkylene, optionally substituted C₂-C₈ alkenylene, optionally substituted C₂-C₈ alkynylene, optionally substituted C₃-C₈ cycloalkylene, optionally substituted 3-8 membered heterocyclylene, optionally substituted arylene, and optionally substituted heteroarylene;

R⁵ and R⁶ are independently selected from optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₈ cycloalkyl, optionally substituted 3-8 membered heterocyclyl, optionally substituted C₂-C₈ alkenyl, optionally substituted C₂-C₈ alkynyl, optionally substituted aryl, and optionally substituted heteroaryl; or

R⁴ and R⁵, R⁴ and R⁶, R⁵ and R⁶ together with the atom to which they are connected form a 3-20 membered cycloalkyl or heterocyclyl ring;

and pharmaceutically acceptable salts thereof.
When the “Linker” moiety of the bivalent compound is attached to R¹;
R² is, at each occurence, independently selected from null, hydrogen, C(O)R⁷, C(O)OR⁷, C(O)NR⁷R⁸, S(O)R⁷, S(O)₂R⁷, S(O)₂NR⁷R⁸, NR⁹C(O)OR⁷, NR⁹C(O)R⁷, NR⁹C(O)NR⁷R⁸, NR⁹S(O)R⁷, NR⁹S(O)₂R⁷, NR⁹S(O)₂NR⁷R⁸, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl; wherein
R⁷, R⁸, and R⁹ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl; or

R⁷ and R⁸, R⁷ and R⁹, R⁸ and R⁹ together with the atom to which they are connected form an optionally substituted 3-20 membered cycloalkyl or heterocyclyl ring; and pharmaceutically acceptable salts thereof.

When the “Linker” moiety of the bivalent compound is attached to R²;
R² is, at each occurence, independently selected from C(O)R⁷, C(O)OR⁷, C(O)NR⁷R⁸, S(O)R⁷, S(O)₂R⁷, S(O)₂NR⁷R⁸, NR⁹C(O)OR⁷, NR⁹C(O)R⁷, NR⁹C(O)NR⁷R⁸, NR⁹S(O)R⁷, NR⁹S(O)₂R⁷, NR⁹S(O)₂NR⁷R⁸, optionally substituted C₁-C₈ alkylene, optionally substituted C₂-C₈ alkenylene, optionally substituted C₂-C₈ alkynylene, optionally substituted C₁-C₈ alkoxylene, optionally substituted C₁-C₈alkoxyC₁-C₈alkylene, optionally substituted C₁-C₈alkylaminoC₁-C₈alkylene, optionally substituted 3-8 membered cycloalkylene, optionally substituted C₃-C₈ cycloalkoxylene, optionally substituted 3-8 membered heterocyclylene, optionally substituted arylene, and optionally substituted heteroarylene; wherein

R⁷ is null, or a bivalent moiety selected from optionally substituted C₁-C₈ alkylene, optionally substituted C₂-C₈ alkenylene, optionally substituted C₂-C₈ alkynylene, optionally substituted C₃-C₈ cycloalkylene, optionally substituted 3-8 membered heterocyclylene, optionally substituted arylene, and optionally substituted heteroarylene;

R⁸ and R⁹ are independently selected from optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₈ cycloalkyl, optionally substituted 3-8 membered heterocyclyl, optionally substituted C₂-C₈ alkenyl, optionally substituted C₂-C₈ alkynyl, optionally substituted aryl, and optionally substituted heteroaryl; or

R⁷ and R⁸, R⁷ and R⁹, R⁸ and R⁹ together with the atom to which they are connected form a 3-20 membered cycloalkyl or heterocyclyl ring;

and pharmaceutically acceptable salts thereof.

In another embodiment, (HPK1) ligands include a moiety according to FORMULA 2:

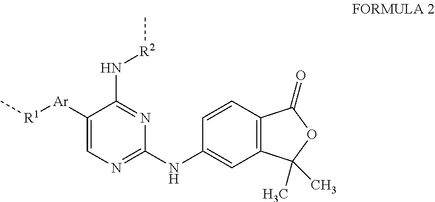

wherein
the “Linker” moiety of the bivalent compound is attached independently to R¹ or R² (indicated by dotted line);
The definitions of, R¹ and R², are the same as for FORMULA 1.
Ar is, at each occurence, independently selected from null, aryl or heteroaryl, each of which is substituted with R¹ and optionally substituted with one or more substituents independently selected from hydrogen, halogen, oxo, CN, NO₂, OR¹², SR¹², NR¹²R¹³, OCOR¹², OCO₂R¹², OCONR¹²R¹³, COR¹², CO₂R¹², CONR¹²R¹³, SOR¹², SO₂R¹², SO₂NR¹²R¹³, NR¹⁴CO₂R¹², NR¹⁴COR¹², NR¹⁴C(O)NR¹²R¹³, NR¹⁴SOR¹², NR¹⁴SO₂R¹², NR¹⁴SO₂NR¹²R¹³, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted 3-8 membered cycloalkyl, optionally substituted C₃-C₈ cycloalkoxy, optionally substituted 4-8 membered heterocyclyl, optionally substituted aryl, and optionally substituted heteroaryl; wherein

R¹², R¹³, and R¹⁴ are independently selected from hydrogen, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted 3-8 membered cycloalkyl, optionally substituted C₃-C₈ cycloalkoxy, optionally substituted 4-8 membered heterocyclyl, optionally substituted aryl, and optionally substituted heteroaryl; or

R¹² and R¹³, R¹² and R¹⁴, R¹³ and R¹⁴ together with the atom to which they are connected form a 4-20 membered heterocyclyl ring;

and pharmaceutically acceptable salts thereof.

In an embodiment, (HPK1) ligands include a moiety according to FORMULA 2A:

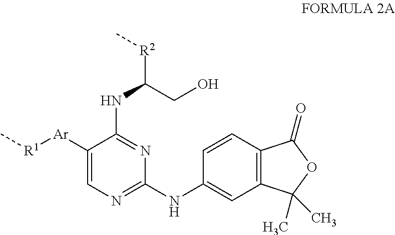

wherein
the “Linker” moiety of the bivalent compound is attached independently to R¹ or R² (indicated by dotted line);
The definitions of, R¹, R² and Ar are the same as for FORMULA 2.
In another embodiment, (HPK1) ligands include a moiety according to FORMULA 2B:

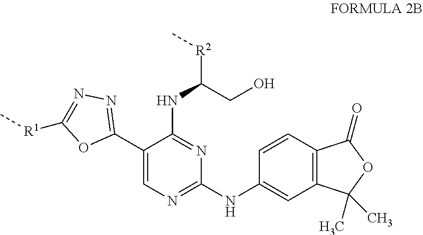

wherein
the “Linker” moiety of the bivalent compound is attached independently to R¹ or R² (indicated by dotted line);
The definitions of, R¹ and R² are the same as for FORMULA 2.
In another embodiment, (HPK1) ligands include a moiety according to FORMULA 2C:

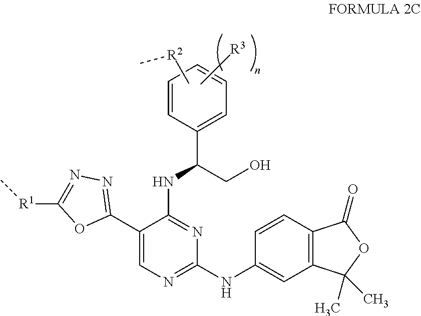

### Wherein

the “Linker” moiety of the bivalent compound is attached independently to R¹ or R² (indicated by dotted line);
The definitions of, R¹ and R² are the same as for FORMULA 2;
R³ is, at each occurence, independently selected from hydrogen, halogen, oxo, CN, NO₂, OR¹⁵, SR¹⁵, NR¹⁵R¹⁶C(O)R¹⁶, C(O)OR¹⁵, C(O)NR¹⁵R¹⁶, S(O)R¹⁵, S(O)₂R¹⁵, S(O)₂NR¹⁵R¹⁶, NR¹⁷C(O)OR¹⁵, NR⁶C(O)R¹⁵, NR¹⁷C(O)NR¹⁵R¹⁶, NR¹⁷S(O)R¹⁵, NR¹⁷S(O)₂R¹⁵, NR¹⁷S(O)₂NR¹⁵R¹⁶, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl; wherein

R¹⁵, R¹⁶, and R¹⁷ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl; or

R¹⁵ and R¹⁶, R¹⁵ and R¹⁷, R¹⁶ and R¹⁷ together with the atom to which they are connected form an optionally substituted 3-20 membered cycloalkyl or heterocyclyl ring;

n is independently selected from 0, 1, 2, 3 and 4;
and pharmaceutically acceptable salts thereof.
In another embodiment, (HPK1) ligands include a moiety according to FORMULA 3:

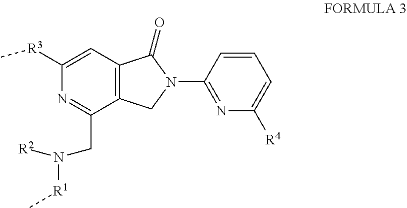

wherein
the “Linker” moiety of the bivalent compound is attached independently to R¹ or R³ (indicated by dotted line);
When the “Linker” moiety of the bivalent compound is attached to R³;
R¹ and R² are, at each occurence, independently selected from null, hydrogen, C(O)R¹⁰, C(O)OR¹⁰, C(O)NR¹⁰R¹¹, S(O)R¹⁰, S(O)₂R¹⁰, S(O)₂NR¹⁰R¹¹, NR¹²C(O)OR¹⁰, NR¹²C(O)R¹⁰, NR¹²C(O)NR¹⁰R¹¹, NR¹²S(O)R¹⁰, NR¹²S(O)₂R¹⁰, NR¹²S(O)₂NR¹⁰R¹¹, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl; wherein
R¹⁰, R¹¹, and R¹² are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl; or

R¹⁰ and R¹¹, R¹⁰ and R¹², R¹¹ and R¹² together with the atom to which they are connected form an optionally substituted 3-20 membered cycloalkyl or heterocyclyl ring;

R¹ and R² are, at each occurence, optionally, together with the atom to which they are connected, form an optionally substituted 3-20 membered cycloalkyl or heterocyclyl ring;
R³ is selected from OR¹³, SR¹³, NR¹³R¹⁴, CH₂NR¹³R¹⁴, CH₂CH₂NR¹³R¹⁴, C₁-C₈ alkylene NR¹³R¹⁴, C(O)R¹³, C(O)OR¹³, C(O)NR¹³R¹⁴, S(O)R¹³, S(O)₂R¹⁴, S(O)₂NR¹³R¹⁴, NR¹⁵C(O)OR¹³, NR¹⁵C(O)R¹³, NR¹⁵C(O)NR¹³R¹⁴, NR¹⁵S(O)R³², NR¹⁵S(O)₂R³², NR¹⁵S(O)₂NR¹³R¹⁴ optionally substituted C₁-C₈ alkylene, optionally substituted C₂-C₈ alkenylene, optionally substituted C₂-C₈ alkynylene, optionally substituted C₁-C₈alkoxyC₁-C₈alkylene, optionally substituted C₁-C₈alkylaminoC₁-C₈alkylene, optionally substituted 3-8 membered cycloalkylene, optionally substituted 3-8 membered heterocyclylene, optionally substituted aryl, and optionally substituted heteroaryl, wherein
R¹³ is null, or a bivalent moiety selected from optionally substituted C₁-C₈ alkylenyl, optionally substituted C₂-C₈ alkenylene, optionally substituted C₂-C₈ alkynylene, optionally substituted C₃-C₈ cycloalkylene, optionally substituted 3-8 membered heterocyclylene, optionally substituted arylene, and optionally substituted heteroarylene;
R¹⁴ and R¹⁵ are independently selected from optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₈ cycloalkyl, optionally substituted 3-8 membered heterocyclyl, optionally substituted C₂-C₈ alkenyl, optionally substituted C₂-C₈ alkynyl, optionally substituted aryl, and optionally substituted heteroaryl; or
R¹³ and R¹⁴, R¹³ and R¹⁵, R¹⁴ and R¹⁵ together with the atom to which they are connected form a 3-20 membered cycloalkyl or heterocyclyl ring;
   R⁴ is, at each occurence, independently selected from hydrogen, halogen, oxo, CN, NO₂, OR¹⁶, SR¹⁶, NR¹⁶R¹⁷, C(O)R¹⁶, C(O)OR¹⁶, C(O)NR¹⁶R¹⁷, S(O)R¹⁶, S(O)₂R¹⁶, S(O)₂NR¹⁶R¹⁷, NR¹⁸C(O)OR¹⁶, NR¹⁸C(O)R¹⁶, NR¹⁸C(O)NR¹⁶R¹⁷, NR¹⁸S(O)R¹⁶, NR¹⁸S(O)₂R¹⁶, NR¹⁸S(O)₂NR¹⁶R¹⁵, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl; wherein

R¹⁶, R¹⁷, and R¹⁸ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl; or

R¹⁶ and R¹⁷, R¹⁶ and R¹⁸, R¹⁷ and R¹⁸ together with the atom to which they are connected form an optionally substituted 3-20 membered cycloalkyl or heterocyclyl ring;

and pharmaceutically acceptable salts thereof.
When the “Linker” moiety of the bivalent compound is attached to R¹;
R¹ is selected from OR¹⁹, SR¹⁹, NR¹⁹R²⁰, CH₂NR¹⁹R²⁰, CH₂CH₂NR¹⁹R²⁰, C₁-C₈ alkylene NR¹⁹R²⁰, C(O)R¹⁹, C(O)OR¹⁹, C(O)NR¹⁹R²⁰, S(O)R¹⁹, S(O)₂R²⁰, S(O)₂NR¹⁹R²⁰, NR²¹C(O)OR¹⁹, NR²¹C(O)R¹⁹, NR²¹C(O)NR¹⁹R²⁰, NR²¹S(O)R¹⁹, NR²¹S(O)₂R¹⁹, NR²¹S(O)₂NR¹⁹R²⁰ optionally substituted C₁-C₈ alkylene, optionally substituted C₂-C₈ alkenylene, optionally substituted C₂-C₈ alkynylene, optionally substituted C₁-C₈alkoxyC₁-C₈alkylene, optionally substituted C₁-C₈alkylaminoC₁-C₈alkylene, optionally substituted 3-8 membered cycloalkylene, optionally substituted 3-8 membered heterocyclylene, optionally substituted aryl, and optionally substituted heteroaryl, wherein
R¹⁹ is null, or a bivalent moiety selected from optionally substituted C₁-C₈ alkylenyl, optionally substituted C₂-C₈ alkenylene, optionally substituted C₂-C₈ alkynylene, optionally substituted C₃-C₈ cycloalkylene, optionally substituted 3-8 membered heterocyclylene, optionally substituted arylene, and optionally substituted heteroarylene;
R²⁰ and R²¹ are independently selected from optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₈ cycloalkyl, optionally substituted 3-8 membered heterocyclyl, optionally substituted C₂-C₈ alkenyl, optionally substituted C₂-C₈ alkynyl, optionally substituted aryl, and optionally substituted heteroaryl; or
R¹⁹ and R²⁰, R¹⁹ and R²¹, R²⁰ and R²¹ together with the atom to which they are connected form a 3-20 membered cycloalkyl or heterocyclyl ring;
   R² is, at each occurence, independently selected from null, hydrogen, C(O)R¹⁰, C(O)OR¹⁰, C(O)NR¹⁰R¹¹, S(O)R¹⁰, S(O)₂R¹⁰, S(O)₂NR¹⁰R¹¹, NR¹²C(O)OR¹⁰, NR¹²C(O)R¹⁰, NR¹²C(O)NR¹⁰R¹¹, NR¹²S(O)R¹⁰, NR¹²S(O)₂R¹⁰, NR¹²S(O)₂NR¹⁰R¹¹, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl; wherein
   R¹⁰, R¹¹, and R¹² are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl; or

R¹⁰ and R¹¹, R¹⁰ and R¹², R¹¹ and R¹² together with the atom to which they are connected form an optionally substituted 3-20 membered cycloalkyl or heterocyclyl ring;

R³ and R⁴ are, at each occurence, independently selected from hydrogen, halogen, oxo, CN, NO₂, OR¹⁶, SR¹⁶, NR¹⁶R¹⁷, C(O)R¹⁶, C(O)OR¹⁶, C(O)NR¹⁶R¹⁷, S(O)R¹⁶, S(O)₂R¹⁶, S(O)₂NR¹⁶R¹⁷, NR¹⁸C(O)OR¹⁶, NR¹⁸C(O)R¹⁶, NR¹⁸C(O)NR¹⁶R¹⁷, NR¹⁸S(O)R¹⁶, NR¹⁸S(O)₂R¹⁶, NR¹⁸S(O)₂NR¹⁶R¹⁵, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl; wherein

R¹⁶, R¹⁷, and R¹⁸ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl; or

R¹⁶ and R¹⁷, R¹⁶ and R¹⁸, R¹⁷ and R¹⁸ together with the atom to which they are connected form an optionally substituted 3-20 membered cycloalkyl or heterocyclyl ring; and pharmaceutically acceptable salts thereof.

In another embodiment, (HPK1) ligands include a moiety according to FORMULA 3′:

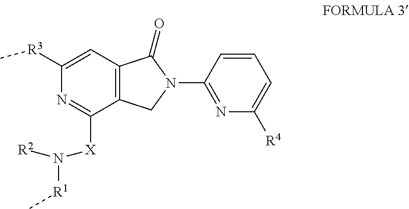

wherein X is absent or when present is CH₂,
the “Linker” moiety of the bivalent compound is attached independently to R¹ or R³ (indicated by dotted line);
when the “Linker” moiety of the bivalent compound is attached to R³; R¹ and R² are, at each occurrence, independently selected from null, hydrogen, C(O)R¹⁰, C(O)OR¹⁰, C(O)NR¹⁰R¹¹, S(O)R¹⁰, S(O)₂R¹⁰, S(O)₂NR¹⁰R¹¹, NR¹²C(O)OR¹⁰, NR¹²C(O)R¹⁰, NR¹²C(O)NR¹⁰R¹¹, NR¹²S(O)R¹⁰, NR¹²S(O)₂R¹⁰, NR¹²S(O)₂NR¹⁰R¹¹, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl; wherein
R¹⁰, R¹¹, and R¹² are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl; or

R¹⁰ and R¹¹, R¹⁰ and R¹², R¹¹ and R¹² together with the atom to which they are connected form an optionally substituted 3-20 membered cycloalkyl or heterocyclyl ring;

R¹ and R² are, at each occurrence, optionally, together with the atom to which they are connected, form an optionally substituted 3-20 membered cycloalkyl or heterocyclyl ring;
R³ is selected from OR¹³, SR¹³, NR¹³R¹⁴, CH₂NR¹³R¹⁴, CH₂CH₂NR¹³R¹⁴, C₁-C₈ alkylene NR¹³R¹⁴, C(O)R¹³, C(O)OR¹³, C(O)NR¹³R¹⁴, S(O)R¹³, S(O)₂R¹⁴, S(O)₂NR¹³R¹⁴, NR¹⁵C(O)OR¹³, NR¹⁵C(O)R¹³, NR¹⁵C(O)NR¹³R¹⁴, NR¹⁵S(O)R³², NR¹⁵S(O)₂R³², NR¹⁵S(O)₂NR¹³R¹⁴ optionally substituted C₁-C₈ alkylene, optionally substituted C₂-C₈ alkenylene, optionally substituted C₂-C₈ alkynylene, optionally substituted C₁-C₈alkoxyC₁-C₈alkylene, optionally substituted C₁-C₈alkylaminoC₁-C₈alkylene, optionally substituted 3-8 membered cycloalkylene, optionally substituted 3-8 membered heterocyclylene, optionally substituted aryl, and optionally substituted heteroaryl, wherein
R¹³ is null, or a bivalent moiety selected from optionally substituted C₁-C₈ alkylenyl, optionally substituted C₂-C₈ alkenylene, optionally substituted C₂-C₈ alkynylene, optionally substituted C₃-C₈ cycloalkylene, optionally substituted 3-8 membered heterocyclylene, optionally substituted arylene, and optionally substituted heteroarylene;
R¹⁴ and R¹⁵ are independently selected from optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₈ cycloalkyl, optionally substituted 3-8 membered heterocyclyl, optionally substituted C₂-C₈ alkenyl, optionally substituted C₂-C₈ alkynyl, optionally substituted aryl, and optionally substituted heteroaryl; or
R¹³ and R¹⁴, R¹³ and R¹⁵, R¹⁴ and R¹⁵ together with the atom to which they are connected form a 3-20 membered cycloalkyl or heterocyclyl ring;
   R⁴ is at each occurrence, independently selected from hydrogen, halogen, oxo, CN, NO₂, OR¹⁶, SR¹⁶, NR¹⁶R¹⁷, C(O)R¹⁶, C(O)OR¹⁶, C(O)NR¹⁶R¹⁷, S(O)R¹⁶, S(O)₂R¹⁶, S(O)₂NR¹⁶R¹⁷, NR¹⁸C(O)OR¹⁶, NR¹⁸C(O)R¹⁶, NR¹⁸C(O)NR¹⁶R¹⁷, NR¹⁸S(O)R¹⁶, NR¹⁸S(O)₂R¹⁶, NR¹⁸S(O)₂NR¹⁶R¹⁵, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl; wherein

R¹⁶, R¹⁷, and R¹⁸ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl; or

R¹⁶ and R¹⁷, R¹⁶ and R¹⁸, R¹⁷ and R¹⁸ together with the atom to which they are connected form an optionally substituted 3-20 membered cycloalkyl or heterocyclyl ring;

when the “Linker” moiety of the bivalent compound is attached to R¹;
R¹ is selected from OR¹⁹, SR¹⁹, NR¹⁹R²⁰, CH₂NR¹⁹R²⁰, CH₂CH₂NR¹⁹R²⁰, C₁-C₈ alkylene NR¹⁹R²⁰, C(O)R¹⁹, C(O)OR¹⁹, C(O)NR¹⁹R²⁰, S(O)R¹⁹, S(O)₂R²⁰, S(O)₂NR¹⁹R²⁰, NR²¹C(O)OR¹⁹, NR²¹C(O)R¹⁹, NR²¹C(O)NR¹⁹R²⁰, NR²¹S(O)R¹⁹, NR²¹S(O)₂R¹⁹, NR²¹S(O)₂NR¹⁹R²⁰ optionally substituted C₁-C₈ alkylene, optionally substituted C₂-C₈ alkenylene, optionally substituted C₂-C₈ alkynylene, optionally substituted C₁-C₈alkoxyC₁-C₈alkylene, optionally substituted C₁-C₈alkylaminoC₁-C₈alkylene, optionally substituted 3-8 membered cycloalkylene, optionally substituted 3-8 membered heterocyclylene, optionally substituted aryl, and optionally substituted heteroaryl, wherein
R¹⁹ is null, or a bivalent moiety selected from optionally substituted C₁-C₈ alkylenyl, optionally substituted C₂-C₈ alkenylene, optionally substituted C₂-C₈ alkynylene, optionally substituted C₃-C₈ cycloalkylene, optionally substituted 3-8 membered heterocyclylene, optionally substituted arylene, and optionally substituted heteroarylene;
R²⁰ and R²¹ are independently selected from optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₈ cycloalkyl, optionally substituted 3-8 membered heterocyclyl, optionally substituted C₂-C₈ alkenyl, optionally substituted C₂-C₈ alkynyl, optionally substituted aryl, and optionally substituted heteroaryl; or
R¹⁹ and R²⁰, R¹⁹ and R²¹, R²⁰ and R²¹ together with the atom to which they are connected form a 3-20 membered cycloalkyl or heterocyclyl ring;
   R² is, at each occurrence, independently selected from null, hydrogen, C(O)R¹⁰, C(O)OR¹⁰, C(O)NR¹⁰R¹¹, S(O)R¹⁰, S(O)₂R¹⁰, S(O)₂NR¹⁰R¹¹, NR¹²C(O)OR¹⁰, NR¹²C(O)R¹⁰, NR¹²C(O)NR¹⁰R¹¹, NR¹²S(O)R¹⁰, NR¹²S(O)₂R¹⁰, NR¹²S(O)₂NR¹⁰R¹¹, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl; wherein
   R¹⁰, R¹¹, and R¹² are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl; or

R¹⁰ and R¹¹, R¹⁰ and R¹², R¹¹ and R¹² together with the atom to which they are connected form an optionally substituted 3-20 membered cycloalkyl or heterocyclyl ring;

R³ and R⁴ are, at each occurrence, independently selected from hydrogen, halogen, oxo, CN, NO₂, OR¹⁶, SR¹⁶, NR¹⁶R¹⁷, C(O)R¹⁶, C(O)OR¹⁶, C(O)NR¹⁶R¹⁷, S(O)R¹⁶, S(O)₂R¹⁶, S(O)₂NR¹⁶R¹⁷, NR¹⁸C(O)OR¹⁶, NR¹⁸C(O)R¹⁶, NR¹⁸C(O)NR¹⁶R¹⁷, NR¹⁸S(O)R¹⁶, NR¹⁸S(O)₂R¹⁶, NR¹⁸S(O)₂NR¹⁶R¹⁵, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl; wherein

R¹⁶, R¹⁷, and R¹⁸ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl; or

R¹⁶ and R¹⁷, R¹⁶ and R¹⁸, R¹⁷ and R¹⁸ together with the atom to which they are connected form an optionally substituted 3-20 membered cycloalkyl or heterocyclyl ring;

In another embodiment, (HPK1) ligands include a moiety according to FORMULA 3A:

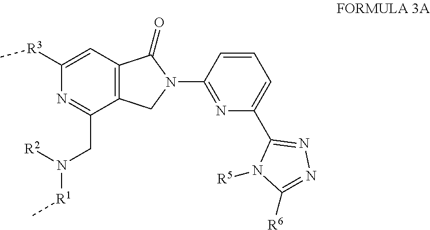

wherein
the “Linker” moiety of the bivalent compound is attached independently to R¹ or R³ (indicated by dotted line);
The definitions of, R¹, R² and R³ are the same as for FORMULA 3;
R⁵ is, at each occurence, independently selected from null, hydrogen, C(O)R²², C(O)OR²², C(O)NR²²R²³, S(O)R²², S(O)₂R²², S(O)₂NR²²R²³, NR²⁴C(O)OR²², NR²⁴C(O)R²², NR²⁴C(O)NR²²R²³, NR²⁴S(O)R²², NR²⁴S(O)₂R²², NR²⁴S(O)₂NR²²R²³, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl; wherein

R²², R²³, and R²⁴ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl; or

R²² and R²³, R²² and R²⁴, R²³ and R²⁴ together with the atom to which they are connected form an optionally substituted 3-20 membered cycloalkyl or heterocyclyl ring;

R⁶ is, at each occurence, independently selected from hydrogen, halogen, oxo, CN, NO₂, OR²⁵, SR²⁵, NR²⁵R²⁶, C(O)R²⁵, C(O)OR²⁵, C(O)NR²⁵R²⁶, S(O)R²⁵, S(O)₂R²⁵, S(O)₂NR²⁵R²⁶, NR²⁷C(O)OR²⁵, NR²⁷C(O)R²⁵, NR²¹C(O)NR²⁵R²⁶, NR²⁷S(O)R²⁵, NR²⁷S(O)₂R²⁵, NR²⁷S(O)₂NR²⁵R²⁶, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl; wherein

R²⁵, R²⁶, and R²⁷ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl; or

R²⁵ and R²⁶, R²⁵ and R²⁶, R²⁶ and R²⁷ together with the atom to which they are connected form an optionally substituted 3-20 membered cycloalkyl or heterocyclyl ring; and pharmaceutically acceptable salts thereof.

In another embodiment, (HIPK1) ligands include a moiety according to FORMULA 3A′:

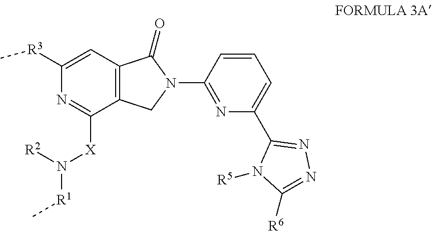

wherein X is absent or when present is CH₂,
the “Linker” moiety of the bivalent compound is attached independently to R¹ or R³ (indicated by dotted line);
the definitions of, R¹, R² and R³ are the same as for FORMULA 3;
R⁵ is, at each occurrence, independently selected from null, hydrogen, C(O)R²², C(O)OR²², C(O)NR²²R²³, S(O)R²², S(O)₂R²², S(O)₂NR²²R²³, NR²⁴C(O)OR²², NR²⁴C(O)R²², NR²⁴C(O)NR²²R²³, NR²⁴S(O)R²², NR²⁴S(O)₂R²², NR²⁴S(O)₂NR²²R²³, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl; wherein

R²², R²³, and R²⁴ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl; or

R²² and R²³, R²² and R²⁴, R²³ and R²⁴ together with the atom to which they are connected form an optionally substituted 3-20 membered cycloalkyl or heterocyclyl ring;

R⁶ is, at each occurrence, independently selected from hydrogen, halogen, oxo, CN, NO₂, OR²⁵, SR²⁵, NR²⁵R²⁶, C(O)R²⁵, C(O)OR²⁵, C(O)NR²⁵R²⁶, S(O)R²⁵, S(O)₂R²⁵, S(O)₂NR²⁵R²⁶, NR²⁷C(O)OR²⁵, NR²⁷C(O)R²⁵, NR²¹C(O)NR²⁵R²⁶, NR²⁷S(O)R²⁵, NR²⁷S(O)₂R²⁵, NR²⁷S(O)₂NR²⁵R²⁶, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl; wherein

R²⁵, R²⁶, and R²⁷ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl; or

R²⁵ and R²⁶, R²⁵ and R²⁶, R²⁶ and R²⁷ together with the atom to which they are connected form an optionally substituted 3-20 membered cycloalkyl or heterocyclyl ring;

In another embodiment, (HPK1) ligands include a moiety according to FORMULA 3B:

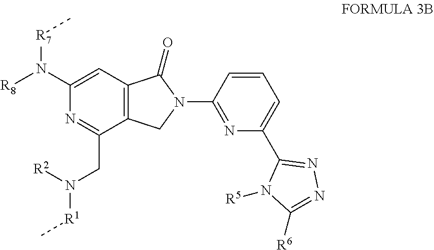

wherein
the “Linker” moiety of the bivalent compound is attached independently to R¹ or R⁷ (indicated by dotted line);
The definitions of R⁵ and R⁶ are the same as for FORMULA 3A;
When the “Linker” moiety of the bivalent compound is attached to R¹;
R¹ is selected from OR²⁸, SR²⁸, NR²⁸R²⁹, CH₂NR²⁸R²⁹, CH₂CH₂NR²⁸R²⁹, C₁-C₈ alkylene NR²⁸R²⁹, C(O)R²⁸, C(O)OR²⁸, C(O)NR²⁸R²⁹, S(O)R²⁸, S(O)₂R²⁹, S(O)₂NR²⁸R²⁹, NR³⁰C(O)OR²⁸, NR³⁰C(O)R²⁸, NR³⁰C(O)NR²⁸R²⁹, NR³⁰S(O)R²⁸, NR³⁰S(O)₂R²⁸, NR³⁰S(O)₂NR²⁸R²⁹ optionally substituted C₁-C₈ alkylene, optionally substituted C₂-C₈ alkenylene, optionally substituted C₂-C₈ alkynylene, optionally substituted C₁-C₈alkoxyC₁-C₈alkylene, optionally substituted C₁-C₈alkylaminoC₁-C₈alkylene, optionally substituted 3-8 membered cycloalkylene, optionally substituted 3-8 membered heterocyclylene, optionally substituted aryl, and optionally substituted heteroaryl, wherein
R²⁸ is null, or a bivalent moiety selected from optionally substituted C₁-C₈ alkylenyl, optionally substituted C₂-C₈ alkenylene, optionally substituted C₂-C₈ alkynylene, optionally substituted C₃-C₈ cycloalkylene, optionally substituted 3-8 membered heterocyclylene, optionally substituted arylene, and optionally substituted heteroarylene;
R²⁹ and R³⁰ are independently selected from optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₈ cycloalkyl, optionally substituted 3-8 membered heterocyclyl, optionally substituted C₂-C₈ alkenyl, optionally substituted C₂-C₈ alkynyl, optionally substituted aryl, and optionally substituted heteroaryl; or
R²⁸ and R²⁹, R²⁸ and R²⁹, R²⁹ and R³⁰ together with the atom to which they are connected form a 3-20 membered cycloalkyl or heterocyclyl ring;
   R², R⁷ and R⁸ are, at each occurence, independently selected from null, hydrogen, C(O)R³¹, C(O)OR³¹, C(O)NR³¹R³², S(O)R³¹, S(O)₂R³¹, S(O)₂NR³¹R³², NR³³C(O)OR³¹, NR³³C(O)R³¹, NR³³C(O)NR³¹R³², NR³³S(O)R³¹, NR³³S(O)₂R³¹, NR³³S(O)₂NR³¹R³², optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl; wherein

R³¹, R³², and R³³ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl; or

R³¹ and R³², R³¹ and R³³, R³² and R³³ together with the atom to which they are connected form an optionally substituted 3-20 membered cycloalkyl or heterocyclyl ring;

R⁷ and R⁸ are, at each occurence, optionally, together with the atom to which they are connected, form an optionally substituted 3-20 membered cycloalkyl or heterocyclyl ring; and pharmaceutically acceptable salts thereof.
When the “Linker” moiety of the bivalent compound is attached to R⁷;
R⁷ is selected from OR²⁸, SR²⁸, NR²⁸R²⁹, CH₂NR²⁸R²⁹, CH₂CH₂NR²⁸R²⁹, C₁-C₈ alkylene NR²⁸R²⁹, C(O)R²⁸, C(O)OR²⁸, C(O)NR²⁸R²⁹, S(O)R²⁸, S(O)₂R²⁹, S(O)₂NR²⁸R²⁹, NR³⁰C(O)OR²⁸, NR³⁰C(O)R²⁸, NR³⁰C(O)NR²⁸R²⁹, NR³⁰S(O)R²⁸, NR³⁰S(O)₂R²⁸, NR³⁰S(O)₂NR²⁸R²⁹ optionally substituted C₁-C₈ alkylene, optionally substituted C₂-C₈ alkenylene, optionally substituted C₂-C₈ alkynylene, optionally substituted C₁-C₈alkoxyC₁-C₈alkylene, optionally substituted C₁-C₈alkylaminoC₁-C₈alkylene, optionally substituted 3-8 membered cycloalkylene, optionally substituted 3-8 membered heterocyclylene, optionally substituted aryl, and optionally substituted heteroaryl, wherein
R²⁸ is null, or a bivalent moiety selected from optionally substituted C₁-C₈ alkylenyl, optionally substituted C₂-C₈ alkenylene, optionally substituted C₂-C₈ alkynylene, optionally substituted C₃-C₈ cycloalkylene, optionally substituted 3-8 membered heterocyclylene, optionally substituted arylene, and optionally substituted heteroarylene;
R²⁹ and R³⁰ are independently selected from optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₈ cycloalkyl, optionally substituted 3-8 membered heterocyclyl, optionally substituted C₂-C₈ alkenyl, optionally substituted C₂-C₈ alkynyl, optionally substituted aryl, and optionally substituted heteroaryl; or
R²⁸ and R²⁹, R²⁸ and R²⁹, R²⁹ and R³⁰ together with the atom to which they are connected form a 3-20 membered cycloalkyl or heterocyclyl ring;
   R¹, R² and R⁸ are, at each occurence, independently selected from null, hydrogen, C(O)R³¹, C(O)OR³¹, C(O)NR³¹R³², S(O)R³¹, S(O)₂R³¹, S(O)₂NR³¹R³², NR³³C(O)OR³¹, NR³³C(O)R³¹, NR³³C(O)NR³¹R³², NR³³S(O)R³¹, NR³³S(O)₂R³¹, NR³³S(O)₂NR³¹R³², optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl; wherein

R³¹, R³², and R³³ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl; or

R³¹ and R³², R³¹ and R³³, R³² and R³³ together with the atom to which they are connected form an optionally substituted 3-20 membered cycloalkyl or heterocyclyl ring;

R¹ and R² are, at each occurence, optionally, together with the atom to which they are connected, form an optionally substituted 3-20 membered cycloalkyl or heterocyclyl ring; and pharmaceutically acceptable salts thereof.
In another embodiment, (HPK1) ligands include a moiety according to FORMULA 4:

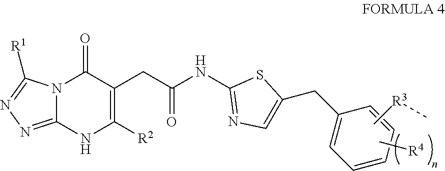

wherein
the “Linker” moiety of the bivalent compound is attached independently to R³ (indicated by dotted line);
R^{1′} R² and R⁴ are, at each occurence, independently selected from hydrogen, halogen, oxo, CN, NO₂, OR⁵, SR⁵, NR⁵R⁶, C(O)R⁵, C(O)OR⁵, C(O)NR⁵R⁶, S(O)R⁵, S(O)₂R⁵, S(O)₂NR⁵R⁶, NR⁷C(O)OR⁵, NR⁷C(O)R⁵, NR⁷C(O)NR²⁵R²⁶, NR⁷S(O)R⁵, NR⁷S(O)₂R⁵, NR⁷S(O)₂NR⁵R⁶ optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl; wherein

R⁵, R⁶, and R⁷ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl; or

R⁵ and R⁶, R⁵ and R⁷, R⁶ and R⁷ together with the atom to which they are connected form an optionally substituted 3-20 membered cycloalkyl or heterocyclyl ring;

n is independently selected from 0, 1, 2, 3 and 4;
R³ is selected from OR⁸, SR⁸, NR⁸R⁹, CH₂NR⁸R⁹, CH₂CH₂NR⁸R⁹, C₁-C₈ alkylene NR⁸R⁹, C(O)R⁸, C(O)OR⁹, C(O)NR⁸R⁹, S(O)R⁸, S(O)₂R⁹, S(O)₂NR⁸R⁹, NR¹⁰C(O)OR⁹, NR¹⁰C(O)R⁹, NR¹⁰C(O)NR⁸R⁹, NR¹⁰S(O)R⁸, NR¹⁰S(O)₂R⁸, NR¹⁰S(O)₂NR⁸R⁹ optionally substituted C₁-C₈ alkylene, optionally substituted C₂-C₈ alkenylene, optionally substituted C₂-C₈ alkynylene, optionally substituted C₁-C₈alkoxyC₁-C₈alkylene, optionally substituted C₁-C₈alkylaminoC₁-C₈alkylene, optionally substituted 3-8 membered cycloalkylene, optionally substituted 3-8 membered heterocyclylene, optionally substituted aryl, and optionally substituted heteroaryl, wherein
R⁸ is null, or a bivalent moiety selected from optionally substituted C₁-C₈ alkylenyl, optionally substituted C₂-C₈ alkenylene, optionally substituted C₂-C₈ alkynylene, optionally substituted C₃-C₈ cycloalkylene, optionally substituted 3-8 membered heterocyclylene, optionally substituted arylene, and optionally substituted heteroarylene;
R⁹ and R¹⁰ are independently selected from optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₈ cycloalkyl, optionally substituted 3-8 membered heterocyclyl, optionally substituted C₂-C₈ alkenyl, optionally substituted C₂-C₈ alkynyl, optionally substituted aryl, and optionally substituted heteroaryl; or
R⁸ and R⁹, R⁸ and R¹⁰, R⁹ and R¹⁰ together with the atom to which they are connected form a 3-20 membered cycloalkyl or heterocyclyl ring;
   and pharmaceutically acceptable salts thereof.
   In another embodiment, (HPK1) ligands include a moiety according to FORMULA 4A:

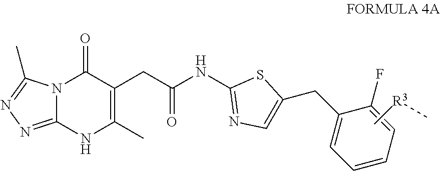

wherein
the “Linker” moiety of the bivalent compound is attached independently to R³ (indicated by dotted line);
The definitions of R³ is the same as for FORMULA 4;

### Degradation/Disruption Tags

Degradation/Disruption tags (EL) include, but are not limited to:

In an embodiment, degradation/disruption tags include a moiety according to FORMULAE 12A, 12B, 12C and 12D:

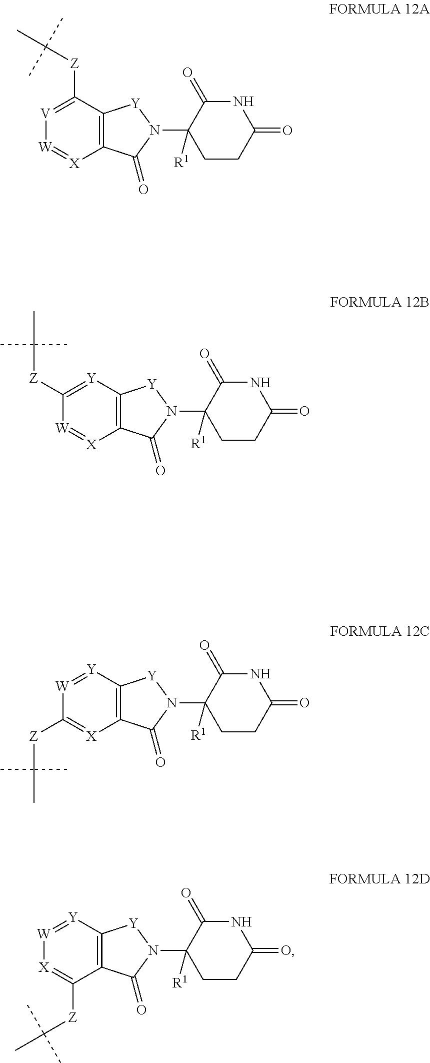

wherein

V, W, and X are independently selected from CR² and N;

Y is selected from CO, CR³R⁴, and N═N;

Z is selected from null, CO, CR⁵R⁶, NR⁵, O, optionally substituted C₁-C₁₀ alkylene, optionally substituted C₁-C₁₀ alkenylene, optionally substituted C₁-C₁₀ alkynylene, optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl; preferably, Z is selected from null, CH₂, CH═CH, C≡C, NH and O;

R¹, and R² are independently selected from hydrogen, halogen, cyano, nitro, optionally substituted C₁-C₆ alkyl, optionally substituted 3 to 6 membered carbocyclyl, and optionally substituted 4 to 6 membered heterocyclyl;

R³, and R⁴ are independently selected from hydrogen, halogen, cyano, nitro, optionally substituted C₁-C₆ alkyl, optionally substituted 3 to 6 membered carbocyclyl, and optionally substituted 4 to 6 membered heterocyclyl; or R³ and R⁴ together with the atom to which they are connected form a 3-6 membered carbocyclyl, or 4-6 membered heterocyclyl; and

R⁵ and R⁶ are independently selected from null, hydrogen, halogen, oxo, hydroxyl, amino, cyano, nitro, optionally substituted C₁-C₆ alkyl, optionally substituted 3 to 6 membered carbocyclyl, and optionally substituted 4 to 6 membered heterocyclyl; or R⁵ and R⁶ together with the atom to which they are connected form a 3-6 membered carbocyclyl, or 4-6 membered heterocyclyl.

In an embodiment, degradation/disruption tags include a moiety according to one of FORMULAE 12E, 12F, 12G, 12H and 12I:

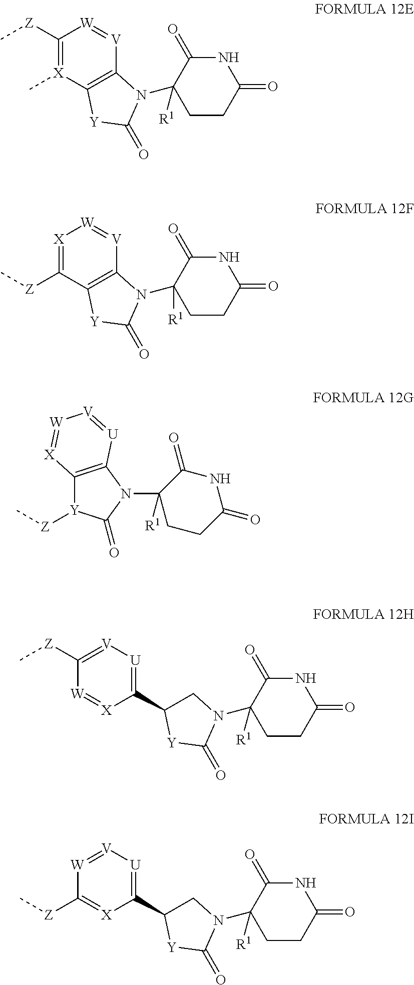

wherein

U, V, W, and X are independently selected from CR² and N;

Y is selected from CR³R⁴, NR³ and O; preferably, Y is selected from CH₂, NH, NCH₃ and O;

Z is selected from null, CO, CR⁵R⁶, NR⁵, O, optionally substituted C₁-C₁₀ alkylene, optionally substituted C₁-C₁₀ alkenylene, optionally substituted C₁-C₁₀ alkynylene, optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl; preferably, Z is selected from null, CH₂, CH═CH, C≡C, NH and O;

R¹, and R² are independently selected from hydrogen, halogen, cyano, nitro, optionally substituted C₁-C₆ alkyl, optionally substituted 3 to 6 membered carbocyclyl, and optionally substituted 4 to 6 membered heterocyclyl;

R³, and R⁴ are independently selected from hydrogen, halogen, cyano, nitro, optionally substituted C₁-C₆ alkyl, optionally substituted 3 to 6 membered carbocyclyl, and optionally substituted 4 to 6 membered heterocyclyl; or R³ and R⁴ together with the atom to which they are connected form a 3-6 membered carbocyclyl, or 4-6 membered heterocyclyl; and

R⁵ and R⁶ are independently selected from null, hydrogen, halogen, oxo, hydroxyl, amino, cyano, nitro, optionally substituted C₁-C₆ alkyl, optionally substituted 3 to 6 membered carbocyclyl, and optionally substituted 4 to 6 membered heterocyclyl; or R⁵ and R⁶ together with the atom to which they are connected form a 3-6 membered carbocyclyl, or 4-6 membered heterocyclyl; and
pharmaceutically acceptable salts thereof.

In an embodiment, degradation/disruption tags include a moiety according to FORMULA 13A:

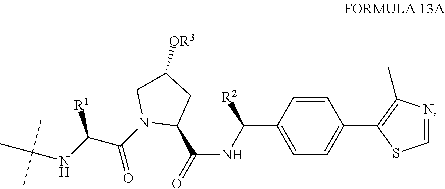

wherein

R¹ and R² are independently selected from hydrogen, optionally substituted C₁-C₈ alkyl, optionally substituted C₁-C₈alkoxyC₁-C₈alkyl, optionally substituted C₁-C₈ haloalkyl, optionally substituted C₁-C₈ hydroxyalkyl, optionally substituted C₁-C₈ aminoalkyl, optionally substituted C₁-C₈alkylaminoC₁-C₈alkyl, optionally substituted C₃-C₇ cycloalkyl, optionally substituted 3-7 membered heterocyclyl, optionally substituted C₂-C₈ alkenyl, and optionally substituted C₂-C₈ alkynyl; and

R³ is hydrogen, optionally substituted C(O)C₁-C₈ alkyl, optionally substituted C(O)C₁-C₈alkoxyC₁-C₈alkyl, optionally substituted C(O)C₁-C₈ haloalkyl, optionally substituted C(O)C₁-C₈ hydroxyalkyl, optionally substituted C(O)C₁-C₈ aminoalkyl, optionally substituted C(O)C₁-C₈alkylaminoC₁-C₈alkyl, optionally substituted C(O)C₃-C₇ cycloalkyl, optionally substituted C(O)(3-7 membered heterocyclyl), optionally substituted C(O)C₂-C₈ alkenyl, optionally substituted C(O)C₂-C₈ alkynyl, optionally substituted C(O)OC₁-C₈alkoxyC₁-C₈alkyl, optionally substituted C(O)OC₁-C₈ haloalkyl, optionally substituted C(O)OC₁-C₈ hydroxyalkyl, optionally substituted C(O)OC₁-C₈ aminoalkyl, optionally substituted C(O)OC₁-C₈alkylaminoC₁-C₈alkyl, optionally substituted C(O)OC₃-C₇ cycloalkyl, optionally substituted C(O)O(3-7 membered heterocyclyl), optionally substituted C(O)OC₂-C₈ alkenyl, optionally substituted C(O)OC₂-C₈ alkynyl, optionally substituted C(O)NC₁-C₈alkoxyC₁-C₈alkyl, optionally substituted C(O)NC₁-C₈ haloalkyl, optionally substituted C(O)NC₁-C₈ hydroxyalkyl, optionally substituted C(O)NC₁-C₈ aminoalkyl, optionally substituted C(O)NC₁-C₈alkylaminoC₁-C₈alkyl, optionally substituted C(O)NC₃-C₇ cycloalkyl, optionally substituted C(O)N(3-7 membered heterocyclyl), optionally substituted C(O)NC₂-C₈ alkenyl, optionally substituted C(O)NC₂-C₈ alkynyl, optionally substituted P(O)(OH)₂, optionally substituted P(O)(OC₁-C₈ alkyl)₂, and optionally substituted P(O)(OC₁-C₈ aryl)₂.

In an embodiment, degradation/disruption tags include a moiety according to FORMULAE 13B, 13C, 13D, 13E and 13F:

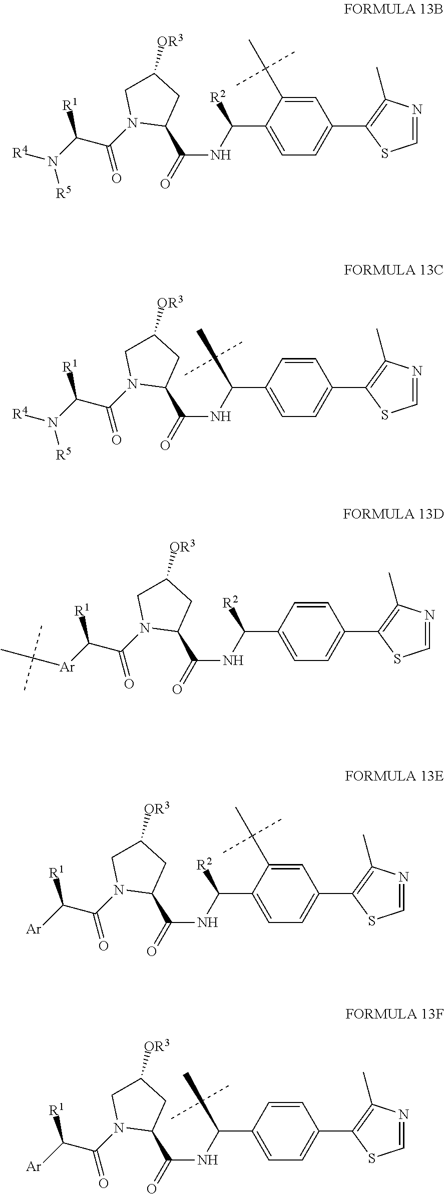

wherein

R¹ and R² are independently selected from hydrogen, halogen, OH, NH₂, CN, optionally substituted C₁-C₈ alkyl, optionally substituted C₁-C₈alkoxyC₁-C₈alkyl, optionally substituted C₁-C₈ haloalkyl, optionally substituted C₁-C₈ hydroxyalkyl, optionally substituted C₁-C₈ aminoalkyl, optionally substituted C₁-C₈alkylaminoC₁-C₈alkyl, optionally substituted C₃-C₇ cycloalkyl, optionally substituted 3-7 membered heterocyclyl, optionally substituted C₂-C₈ alkenyl, and optionally substituted C₂-C₈ alkynyl; (preferably, R¹ is selected from iso-propyl or tert-butyl; and R² is selected from hydrogen or methyl);

R³ is hydrogen, optionally substituted C(O)C₁-C₈ alkyl, optionally substituted C(O)C₁-C₈alkoxyC₁-C₈alkyl, optionally substituted C(O)C₁-C₈ haloalkyl, optionally substituted C(O)C₁-C₈ hydroxyalkyl, optionally substituted C(O)C₁-C₈ aminoalkyl, optionally substituted C(O)C₁-C₈alkylaminoC₁-C₈alkyl, optionally substituted C(O)C₃-C₇ cycloalkyl, optionally substituted C(O)(3-7 membered heterocyclyl), optionally substituted C(O)C₂-C₈ alkenyl, optionally substituted C(O)C₂-C₈ alkynyl, optionally substituted C(O)OC₁-C₈alkoxyC₁-C₈alkyl, optionally substituted C(O)OC₁-C₈ haloalkyl, optionally substituted C(O)OC₁-C₈ hydroxyalkyl, optionally substituted C(O)OC₁-C₈ aminoalkyl, optionally substituted C(O)OC₁-C₈alkylaminoC₁-C₈alkyl, optionally substituted C(O)OC₃-C₇ cycloalkyl, optionally substituted C(O)O(3-7 membered heterocyclyl), optionally substituted C(O)OC₂-C₈ alkenyl, optionally substituted C(O)OC₂-C₈ alkynyl, optionally substituted C(O)NC₁-C₈alkoxyC₁-C₈alkyl, optionally substituted C(O)NC₁-C₈ haloalkyl, optionally substituted C(O)NC₁-C₈ hydroxyalkyl, optionally substituted C(O)NC₁-C₈ aminoalkyl, optionally substituted C(O)NC₁-C₈alkylaminoC₁-C₈alkyl, optionally substituted C(O)NC₃-C₇ cycloalkyl, optionally substituted C(O)N(3-7 membered heterocyclyl), optionally substituted C(O)NC₂-C₈ alkenyl, optionally substituted C(O)NC₂-C₈ alkynyl, optionally substituted P(O)(OH)₂, optionally substituted P(O)(OC₁-C₈ alkyl)₂, and optionally substituted P(O)(OC₁-C₈ aryl)₂; and
R⁴ and R⁵ are independently selected from hydrogen, COR⁶, CO₂R⁶, CONR⁶R⁷, SOR⁶, SO₂R⁶, SO₂NR⁶R⁷, optionally substituted C₁-C₈ alkyl, optionally substituted C₁-C₈alkoxyC₁-C₈alkyl, optionally substituted C₁-C₈alkylaminoC₁-C₈alkyl, optionally substituted 3-8 membered cycloalkyl, optionally substituted 3-8 membered heterocyclyl, optionally substituted aryl, and optionally substituted heteroaryl; wherein
R⁶ and R⁷ are independently selected from hydrogen, optionally substituted C₁-C₈ alkyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted 3-8 membered cycloalkyl, optionally substituted 3-8 membered heterocyclyl, optionally substituted aryl, and optionally substituted heteroaryl; or
R⁴ and R⁵; R⁶ and R⁷ together with the atom to which they are connected form a 4-8 membered cycloalkyl or heterocyclyl ring;
Ar is selected from aryl and heteroaryl, each of which is optionally substituted with one or more substituents independently selected from F, Cl, CN, NO₂, OR⁸, NR⁸R⁹, COR⁸, CO₂R⁸, CONR⁸R⁹, SOR⁸, SO₂R⁸, SO₂NR⁹R¹⁰, NR⁹COR¹⁰, NR⁸C(O)NR⁹R¹⁰, NR⁹SOR¹⁰, NR⁹SO₂R¹⁰, optionally substituted C₁-C₆ alkyl, optionally substituted C₁-C₆ alkoxyalkyl, optionally substituted C₁-C₆ haloalkyl, optionally substituted C₁-C₆ hydroxyalkyl, optionally substituted C₁-C₆alkylaminoC₁-C₆alkyl, optionally substituted C₃-C₇ cycloalkyl, optionally substituted 3-7 membered heterocyclyl, optionally substituted C₂-C₆ alkenyl, optionally substituted C₂-C₆ alkynyl, optionally substituted aryl, and optionally substituted C₄-C₈ heteroaryl; wherein
R⁸, R⁹, and R¹⁰ are independently selected from null, hydrogen, optionally substituted C₁-C₆ alkyl, optionally substituted C₂-C₆ alkenyl, optionally substituted C₂-C₆ alkynyl, optionally substituted C₃-C₇ cycloalkyl, optionally substituted 3-7 membered heterocyclyl, optionally substituted aryl, and optionally substituted heteroaryl; or
R⁸ and R⁹; R⁹ and R¹⁰ together with the atom to which they are connected form a 4-8 membered cycloalkyl or heterocyclyl ring; and

pharmaceutically acceptable salts thereof.

In an embodiment, degradation/disruption tags include a moiety according to FORMULA 14A:

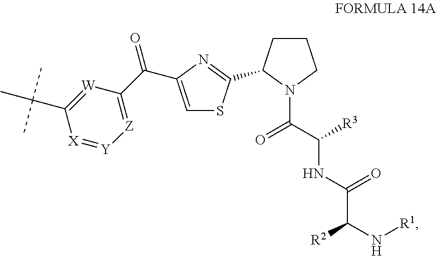

wherein

V, W, X, and Z are independently selected from CR⁴ and N;

R¹, R², R³, and R⁴ are independently selected from hydrogen, optionally substituted C₁-C₈ alkyl, optionally substituted C₁-C₈alkoxyC₁-C₈alkyl, optionally substituted C₁-C₈ haloalkyl, optionally substituted C₁-C₈ hydroxyalkyl, optionally substituted C₃-C₇ cycloalkyl, optionally substituted 3-7 membered heterocyclyl, optionally substituted C₂-C₈ alkenyl, and optionally substituted C₂-C₈ alkynyl.

In an embodiment, degradation/disruption tags include a moiety according to FORMULA 14B:

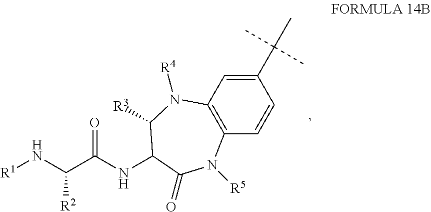

wherein

R¹, R², and R³ are independently selected from hydrogen, halogene, optionally substituted C₁-C₈ alkyl, optionally substituted C₁-C₈alkoxyC₁-C₈alkyl, optionally substituted C₁-C₈ haloalkyl, optionally substituted C₁-C₈ hydroxyalkyl, optionally substituted C₃-C₇ cycloalkyl, optionally substituted 3-7 membered heterocyclyl, optionally substituted C₂-C₈ alkenyl, and optionally substituted C₂-C₈ alkynyl;
R⁴ and R⁵ are independently selected from hydrogen, COR⁶, CO₂R⁶, CONR⁶R⁷, SOR⁶, SO₂R⁶, SO₂NR⁶R⁷, optionally substituted C₁-C₈ alkyl, optionally substituted C₁-C₈alkoxyC₁-C₈alkyl, optionally substituted C₁-C₈alkylaminoC₁-C₈alkyl, optionally substituted aryl-C₁-C₈alkyl, optionally substituted 3-8 membered cycloalkyl, optionally substituted 3-8 membered heterocyclyl, optionally substituted aryl, and optionally substituted heteroaryl; wherein
R⁶ and R⁷ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted 3-8 membered cycloalkyl, optionally substituted 3-8 membered heterocyclyl, optionally substituted aryl, and optionally substituted heteroaryl; or
R⁶ and R⁷ together with the atom to which they are connected form a 4-8 membered cycloalkyl or heterocyclyl ring; and

pharmaceutically acceptable salts thereof.

In an embodiment, degradation/disruption tags are selected from the group consisting of:

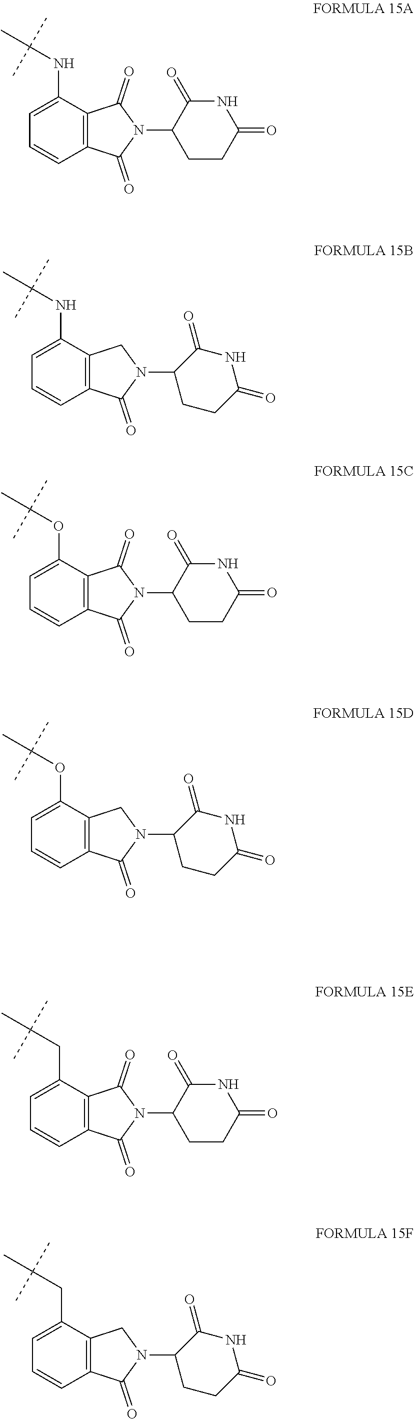

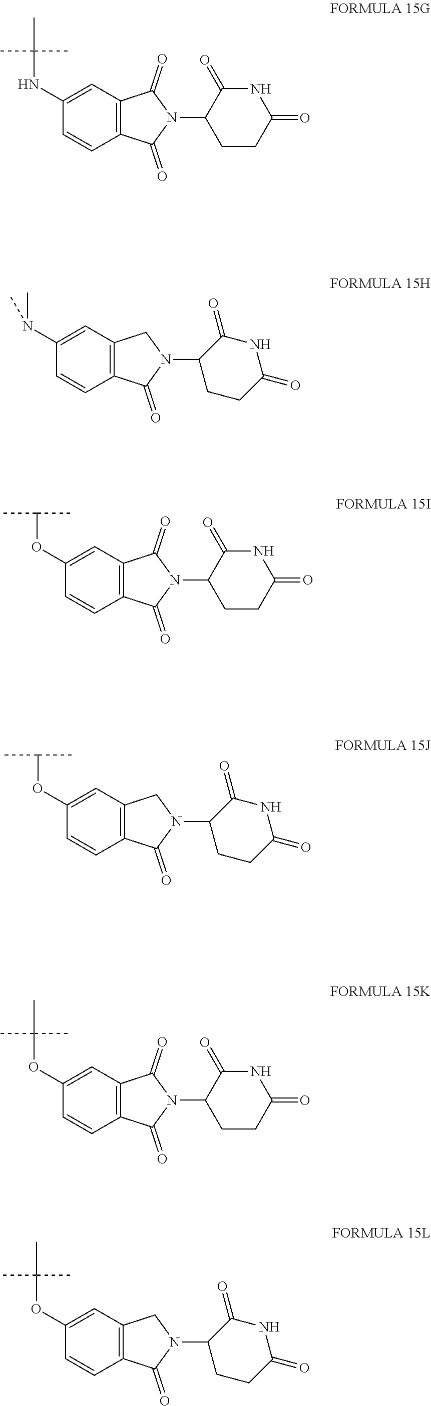

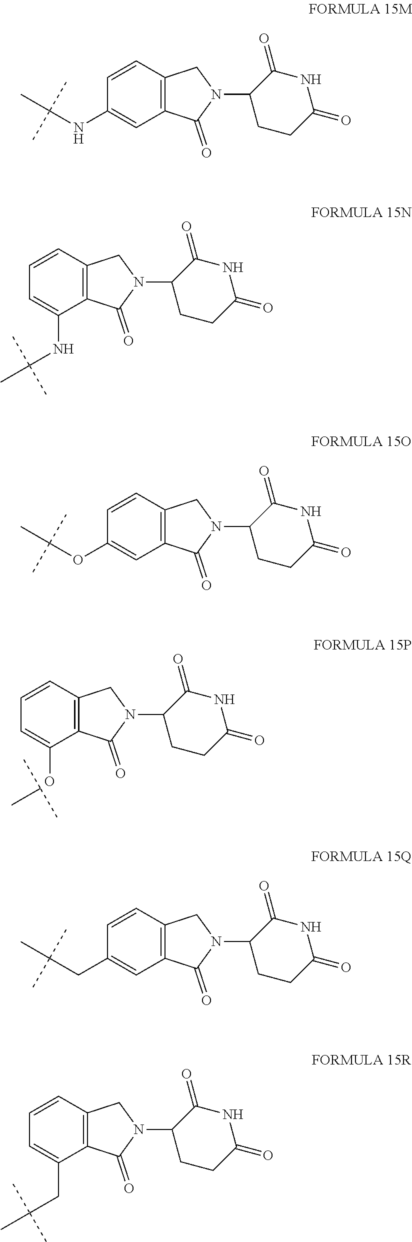

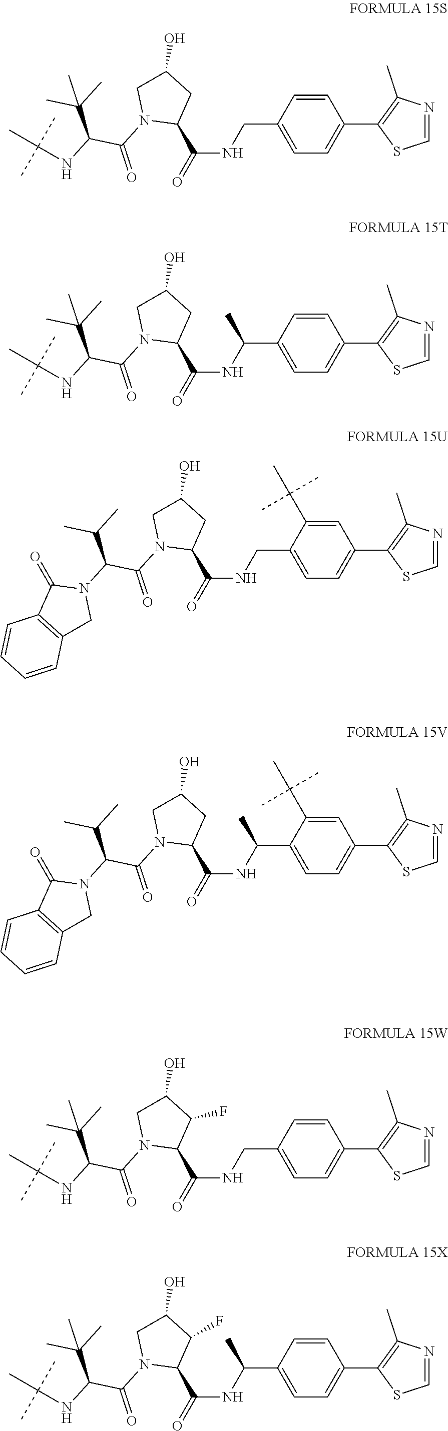

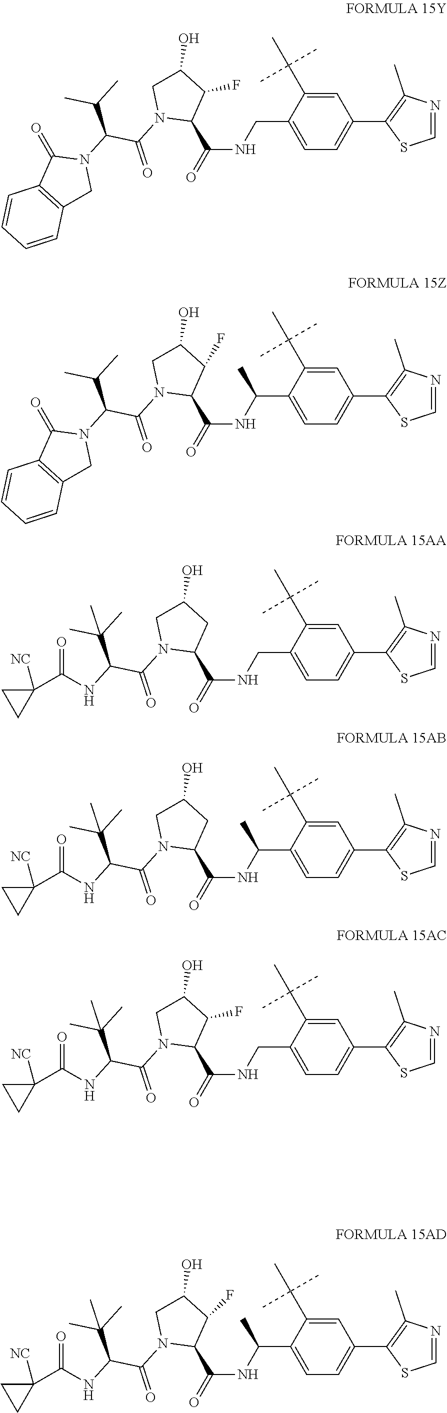

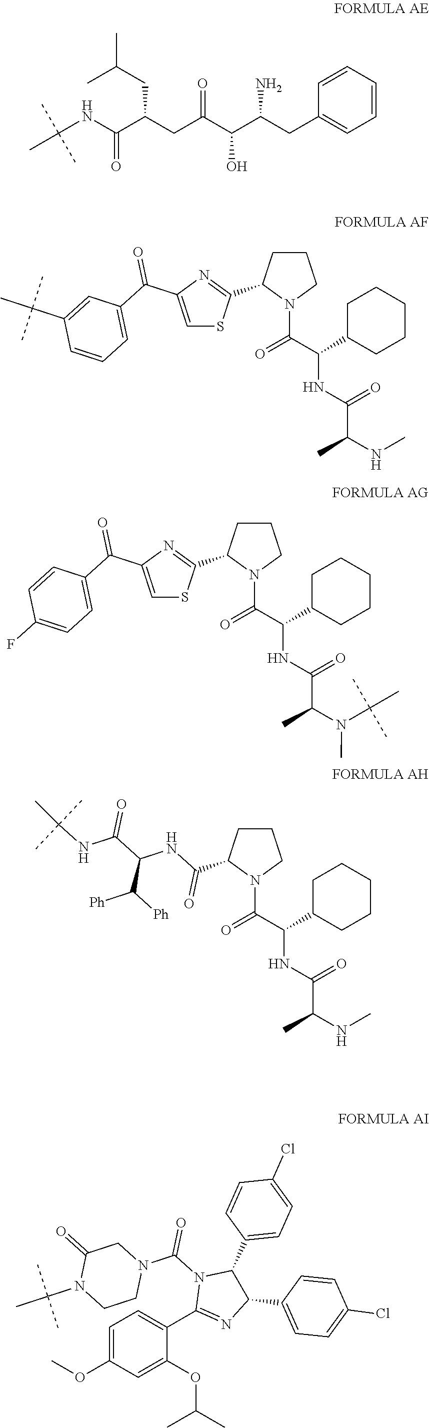

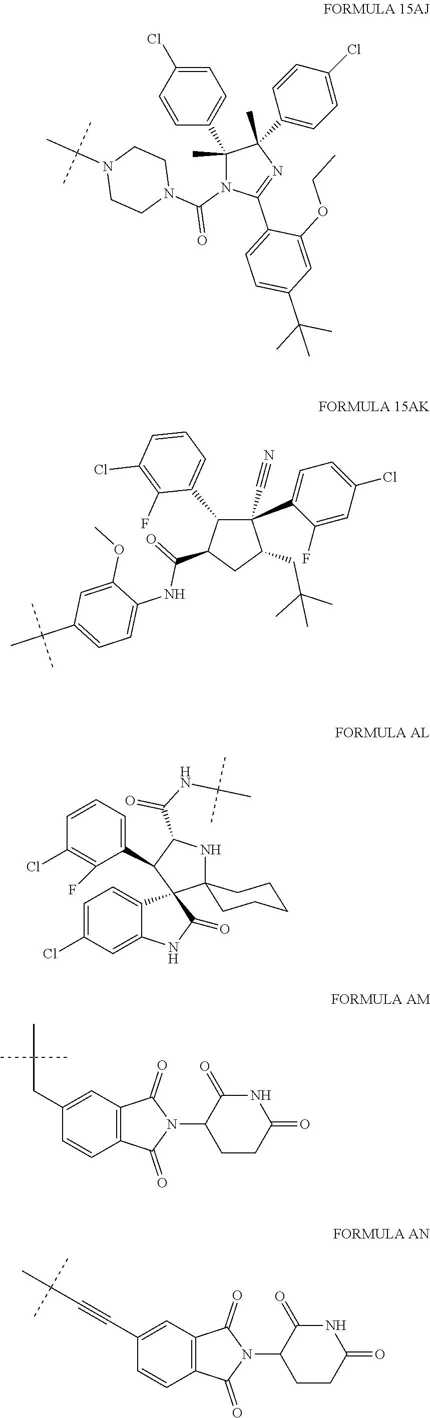

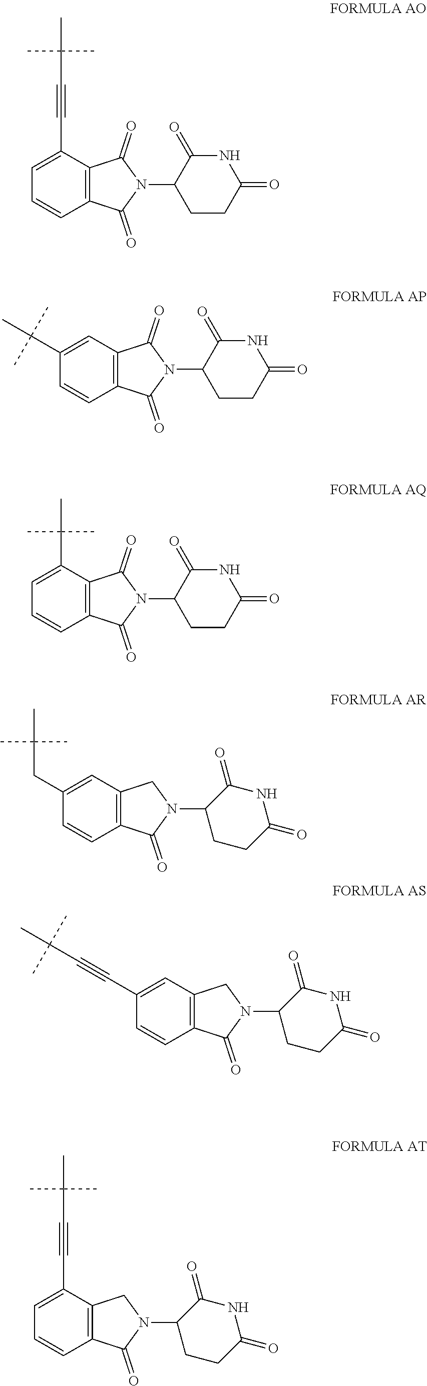

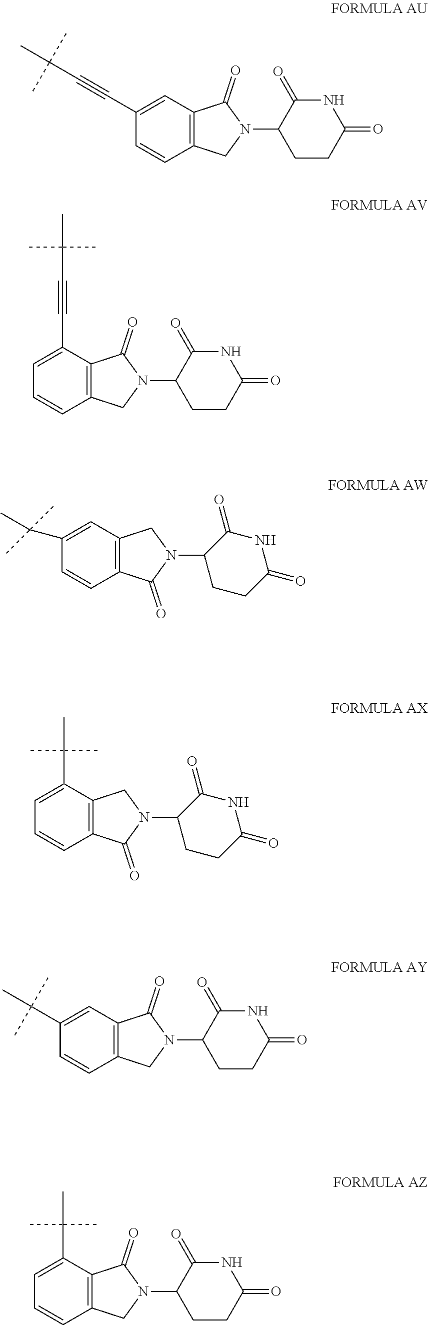

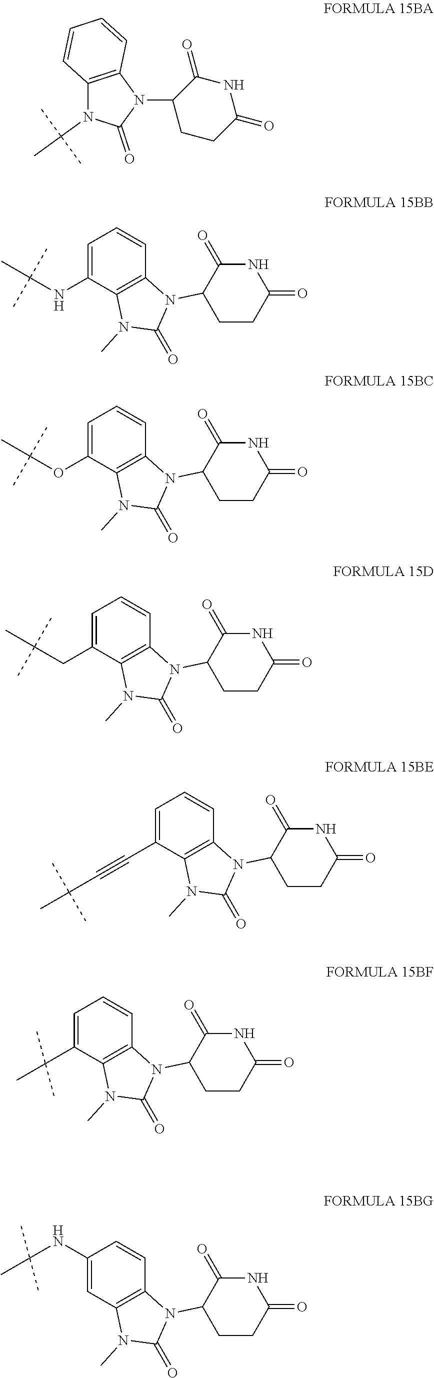

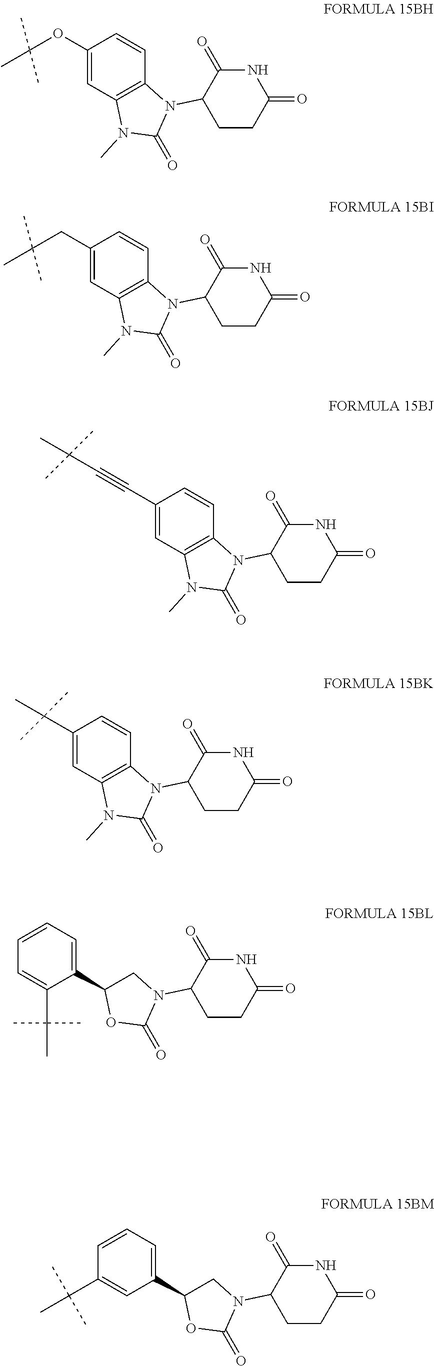

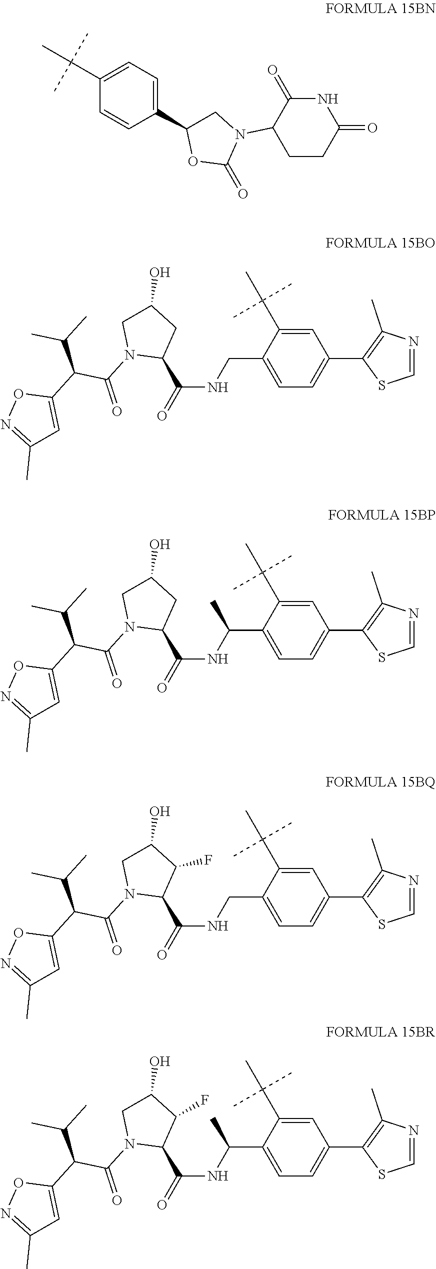

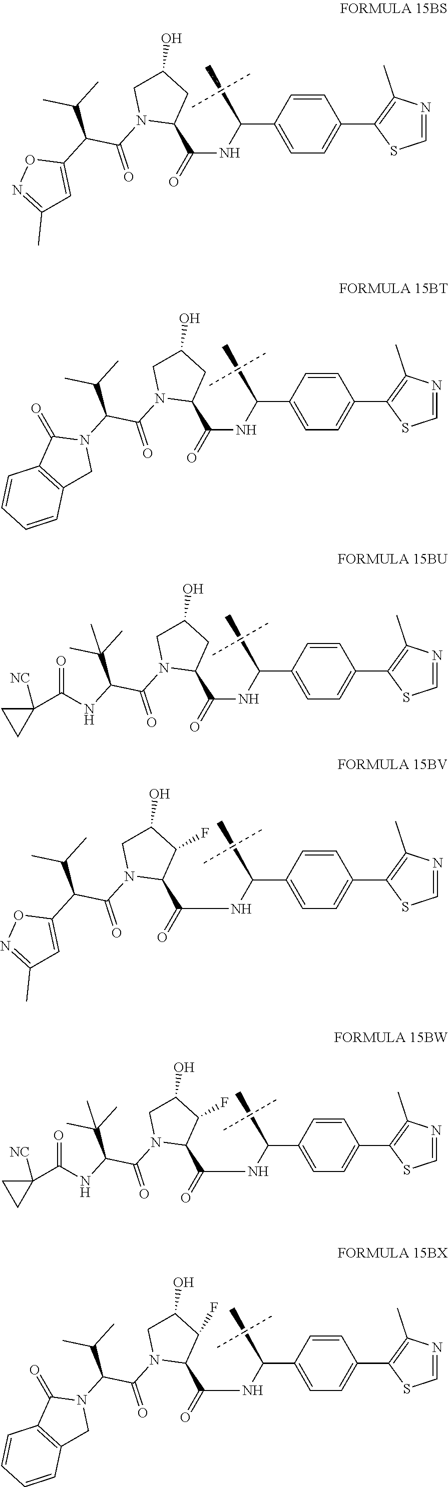

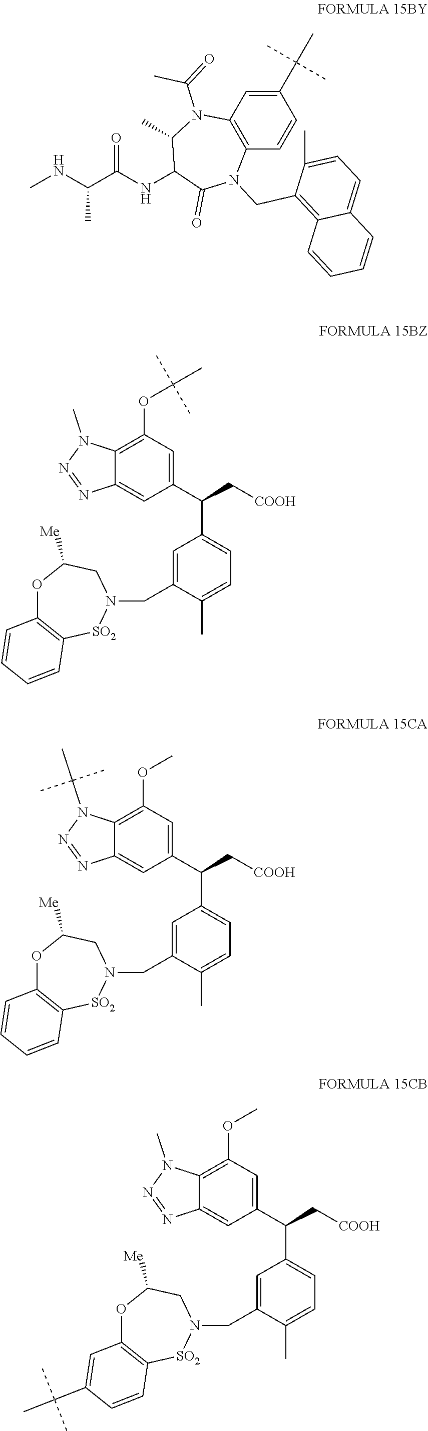

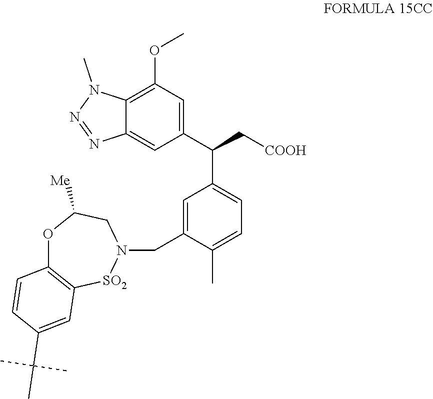

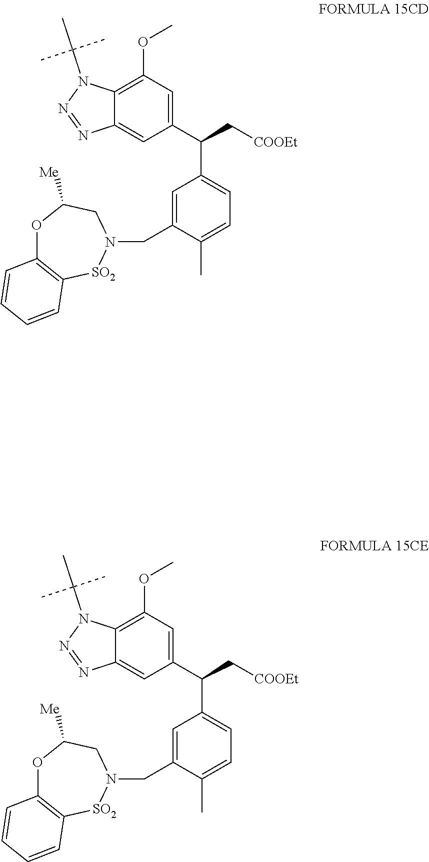

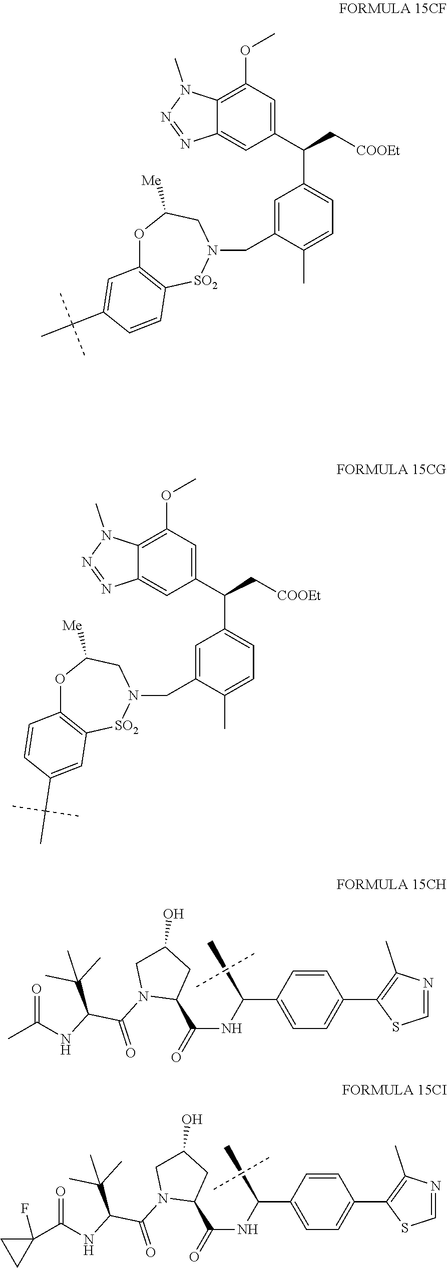

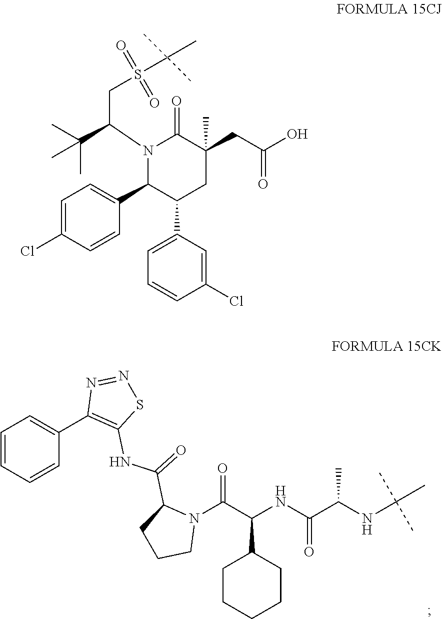

and
pharmaceutically acceptable salts thereof.

### Linkers

In any of the above-described compounds, the HPK1 ligand can be conjugated to the degradation/disruption tag through a linker. The linker can include, e.g., acyclic or cyclic saturated or unsaturated carbon, ethylene glycol, amide, amino, ether, urea, carbamate, aromatic, heteroaromatic, heterocyclic, and/or carbonyl containing groups with different lengths.

In an embodiment, the linker is a moiety according to FORMULA 16:

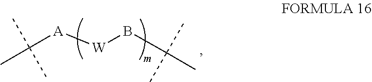

wherein

A, W, and B, at each occurrence, are independently selected from null, CO, CO₂, C(O)NR¹, C(S)NR¹, O, S, SO, SO₂, SO₂NR¹, NR¹, NR⁷CO, NR¹CONR², NR⁷C(S), optionally substituted C₁-C₈ alkyl, optionally substituted C₁-C₈ alkoxy, optionally substituted C₁-C₈alkoxyC₁-C₈alkyl, optionally substituted C₁-C₈ haloalkyl, optionally substituted C₁-C₈ hydroxyalkyl, optionally substituted C₂-C₈ alkenyl, optionally substituted C₂-C₈ alkynyl, optionally substituted 3-8 membered cycloalkyl, optionally substituted C₃-C₈ cycloalkoxy, optionally substituted 3-8 membered heterocyclyl, optionally substituted aryl, optionally substituted heteroaryl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, and optionally substituted C₃-C₁₃ spiro heterocyclyl; wherein

R¹ and R² are independently selected from hydrogen, optionally substituted C₁-C₈ alkyl, optionally substituted C₃-C₈ cycloalkyl, optionally substituted 3-8 membered cycloalkoxy, optionally substituted 3-8 membered heterocyclyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, and optionally substituted C₁-C₈alkylaminoC₁-C₈alkyl; and

m is 0 to 15.

In an embodiment, the linker is a moiety according to FORMULA 16A:

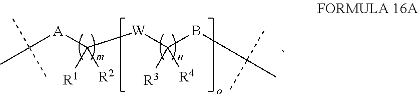

wherein

R¹, R², R³, and R⁴, at each occurrence, are independently selected from hydrogen, halogen, CN, OH, NH₂, optionally substituted C₁-C₈ alkyl, optionally substituted C₃-C₈ cycloalkyl, optionally substituted C₃-C₈ cycloalkoxy, optionally substituted 3-8 membered membered heterocyclyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, and optionally substituted C₁-C₈ alkylaminoC₁-C₈ alkyl;

A, W, and B, at each occurrence, are independently selected from null, CO, CO₂, C(O)NR⁵, C(S)NR⁵, O, S, SO, SO₂, SO₂NR⁵, NR⁵, NR⁵CO, NR⁵CONR⁶, NR⁵C(S), optionally substituted C₁-C₈ alkyl, optionally substituted C₁-C₈ alkoxy, optionally substituted C₁-C₈alkoxyC₁-C₈alkyl, optionally substituted C₁-C₈ haloalkyl, optionally substituted C₁-C₈ hydroxyalkyl, optionally substituted C₂-C₈ alkenyl, optionally substituted C₂-C₈ alkynyl, optionally substituted 3-8 membered cycloalkyl, optionally substituted C₃-C₈ cycloalkoxy, optionally substituted 3-8 membered heterocyclyl, optionally substituted aryl, optionally substituted heteroaryl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, and optionally substituted C₃-C₁₃ spiro heterocyclyl; wherein

R⁵ and R⁶ are independently selected from hydrogen, optionally substituted C₁-C₈ alkyl, optionally substituted 3-8 membered cycloalkyl, optionally substituted C₃-C₈ cycloalkoxy, optionally substituted 3-8 membered heterocyclyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, and optionally substituted C₁-C₈alkylaminoC₁-C₈alkyl;

m is 0 to 15;

n, at each occurrence, is 0 to 15;

o is 0 to 15.

In an embodiment, the linker is a moiety according to FORMULA 16B:

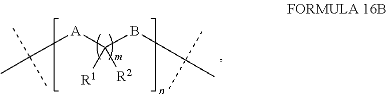

wherein

R¹ and R², at each occurrence, are independently selected from hydrogen, halogen, CN, OH, NH₂, and optionally substituted C₁-C₈ alkyl, optionally substituted 3-8 membered cycloalkyl, optionally substituted C₃-C₈ cycloalkoxy, optionally substituted 3-8 membered heterocyclyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, or C₁-C₈alkylaminoC₁-C₈alkyl;

A and B, at each occurrence, are independently selected from null, CO, CO₂, C(O)NR³, C(S)NR³, O, S, SO, SO₂, SO₂NR³, NR³, NR³CO, NR³CONR⁴, NR³C(S), and optionally substituted C₁-C₈ alkyl, optionally substituted C₁-C₈ alkoxy, optionally substituted C₁-C₈alkoxyC₁-C₈alkyl, optionally substituted C₁-C₈ haloalkyl, optionally substituted C₁-C₈ hydroxyalkyl, optionally substituted C₂-C₈ alkenyl, optionally substituted C₂-C₈ alkynyl, optionally substituted 3-8 membered cycloalkyl, optionally substituted C₃-C₈ cycloalkoxy, optionally substituted 3-8 membered heterocyclyl, optionally substituted aryl, optionally substituted heteroaryl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, or C₃-C₁₃ spiro heterocyclyl; wherein

R³ and R⁴ are independently selected from hydrogen, and optionally substituted C₁-C₈ alkyl, optionally substituted 3-8 membered cycloalkyl, optionally substituted C₃-C₈ cycloalkoxy, optionally substituted 3-8 membered heterocyclyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, or C₁-C₈alkylaminoC₁-C₈alkyl;

each m is 0 to 15; and

n is 0 to 15.

In an embodiment, the linker is a moiety according to FORMULA 16C:

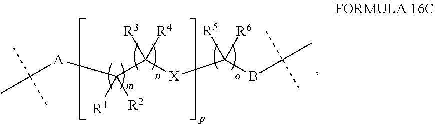

wherein

X is selected from O, NH, and NR⁷;

R¹, R², R³, R⁴, R⁵, and R⁶, at each occurrence, are independently selected from hydrogen, halogen, CN, OH, NH₂, optionally substituted C₁-C₈ alkyl, optionally substituted 3-8 membered cycloalkyl, optionally substituted C₃-C₈ cycloalkoxy, optionally substituted 3-8 membered heterocyclyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, and optionally substituted C₁-C₈ alkylaminoC₁-C₈ alkyl;

A and B, at each occurrence, are independently selected from null, CO, NH, NH—CO, CO—NH, CH₂—NH—CO, CH₂—CO—NH, NH—CO—CH₂, CO—NH—CH₂, CH₂—NH—CH₂—CO—NH, CH₂—NH—CH₂—NH—CO, —CO—NH, CO—NH—CH₂—NH—CH₂, CH₂—NH—CH₂, CO₂, C(O)NR⁷, C(S)NR⁷, O, S, SO, SO₂, SO₂NR⁷, NR⁷, NR⁷CO, NR⁷CONR¹, NR⁷C(S), optionally substituted C₁-C₈ alkyl, optionally substituted C₁-C₈ alkoxy, optionally substituted C₁-C₈alkoxyC₁-C₈alkyl, optionally substituted C₁-C₈ haloalkyl, optionally substituted C₁-C₈ hydroxyalkyl, optionally substituted C₂-C₈ alkenyl, optionally substituted C₂-C₈ alkynyl, optionally substituted 3-8 membered cycloalkyl, optionally substituted C₃-C₈ cycloalkoxy, optionally substituted 3-8 membered heterocyclyl, optionally substituted aryl, optionally substituted heteroaryl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, and optionally substituted C₃-C₁₃ spiro heterocyclyl; wherein

R⁷ and R⁸ are independently selected from hydrogen, optionally substituted C₁-C₈ alkyl, optionally substituted 3-8 membered cycloalkyl, optionally substituted C₃-C₈ cycloalkoxy, optionally substituted 3-8 membered heterocyclyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, and optionally substituted C₁-C₈ alkylaminoC₁-C₈ alkyl;

m, at each occurrence, is 0 to 15;

n, at each occurrence, is 0 to 15;

o is 0 to 15; and

p is 0 to 15; and

pharmaceutically acceptable salts thereof.

In an embodiment, the linker is selected from the group consisting of a ring selected from the group consisting of a 3 to 13 membered ring; a 3 to 13 membered fused ring; a 3 to 13 membered bridged ring; and a 3 to 13 membered spiro ring; and pharmaceutically acceptable salts thereof.

In an embodiment, the linker is a moiety according to one of FORMULAE C1, C2, C3, C4 and C5:

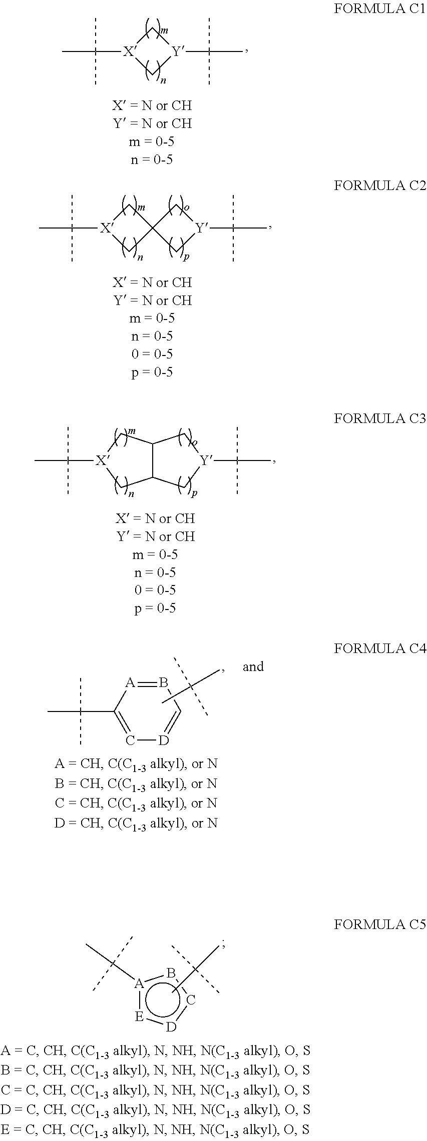

and pharmaceutically acceptable salts thereof.
In an embodiment, HPK1 degrader according to the present invention is selected from the group consist of:
MS-5-006, MS-5-007, MS-5-008, MS-5-009, MS-5-010, MS-5-011, MS-5-015, MS-5-016, MS-5-017, MS-5-018, MS-5-019, MS-5-021, MS-5-022, MS-5-024, MS-5-026, MS-5-027, MS-5-028, MS-5-029, MS-5-030, MS-5-033, MS-5-034, MS-5-035, MS-5-036, MS-5-037, MS-5-038, MS-5-039, MS-5-040, MS-5-041, MS-5-042, MS-5-043, MS-5-044, MS-5-045, MS-5-046, MS-6-003, MS-6-004, MS-6-005, MS-6-006, MS-6-007, MS-6-008, MS-6-009, MS-6-013, MS-6-014, MS-6-015, MS-6-016, MS-6-017, MS-6-018, MS-6-019, MS-6-020, MS-6-021, MS-6-022, MS-6-023, MS-6-024, MS-6-025, MS-6-026, MS-6-027, MS-6-028, MS-6-029, MS-6-037, MS-6-038, MS-6-039, MS-6-040, MS-6-041, MS-6-043, MS-6-044, MS-6-045, MS-6-046, MS-6-047, MS-6-048, MS-6-049, MS-6-050, MS-6-051, MS-6-052, MS-6-053, MS-6-054, MS-6-055, MS-6-058, MS-6-059, MS-6-060, MS-6-061.
In an embodiment, HPK1 degrader according to the present invention is selected from the group consist of:
MS-5-006, MS-5-007, MS-5-008, MS-5-009, MS-5-010, MS-5-011, MS-5-015, MS-5-016, MS-5-017, MS-5-018, MS-5-019, MS-5-021, MS-5-022, MS-5-024, MS-5-026, MS-5-027, MS-5-028, MS-5-029, MS-5-030, MS-5-033, MS-5-034, MS-5-035, MS-5-036, MS-5-037, MS-5-038, MS-5-039, MS-5-040, MS-5-041, MS-5-042, MS-5-043, MS-5-044, MS-5-045, MS-5-046.
In an embodiment, HPK1 degrader according to the present invention is selected from the group consist of:
MS-6-003, MS-6-004, MS-6-005, MS-6-006, MS-6-007, MS-6-008, MS-6-009, MS-6-013, MS-6-014, MS-6-015, MS-6-016, MS-6-017, MS-6-018, MS-6-019, MS-6-020, MS-6-021, MS-6-022, MS-6-023, MS-6-024, MS-6-025, MS-6-026, MS-6-027, MS-6-028, MS-6-029, MS-6-037, MS-6-038, MS-6-039, MS-6-040, MS-6-041, MS-6-043, MS-6-044, MS-6-045, MS-6-046, MS-6-047, MS-6-048, MS-6-049, MS-6-050, MS-6-051, MS-6-052, MS-6-053, MS-6-054, MS-6-055, MS-6-058, MS-6-059, MS-6-060, MS-6-061.
In one embodiment preferred compounds according to the present invention include:
a) 2-(4-(3-(5-(2-((3,3-dimethyl-1-oxo-1,3-dihydroisobenzofuran-5-yl)amino)-4-(((S)-2-hydroxy-1-phenylethyl)amino)pyrimidin-5-yl)-1,3,4-oxadiazol-2-yl)propyl)piperazin-1-yl)-N-(5-((2-(2,6-dioxopiperidin-3-yl)-1,3-dioxoisoindolin-4-yl)amino)pentyl)acetamide (MS-5-007).
b) 2-(4-(3-(5-(2-((3,3-dimethyl-1-oxo-1,3-dihydroisobenzofuran-5-yl)amino)-4-(((S)-2-hydroxy-1-phenylethyl)amino)pyrimidin-5-yl)-1,3,4-oxadiazol-2-yl)propyl)piperazin-1-yl)-N-(8-((2-(2,6-dioxopiperidin-3-yl)-1,3-dioxoisoindolin-4-yl)amino)octyl)acetamide (MS-5-008).
c) 4-((5-(5-(2-((3,3-dimethyl-1-oxo-1,3-dihydroisobenzofuran-5-yl)amino)-4-(((S)-2-hydroxy-1-phenylethyl)amino)pyrimidin-5-yl)-1,3,4-oxadiazol-2-yl)pentyl)amino)-2-(2,6-dioxopiperidin-3-yl)isoindoline-1,3-dione (MS-5-009).
d) 4-((6-(5-(2-((3,3-dimethyl-1-oxo-1,3-dihydroisobenzofuran-5-yl)amino)-4-(((S)-2-hydroxy-1-phenylethyl)amino)pyrimidin-5-yl)-1,3,4-oxadiazol-2-yl)hexyl)amino)-2-(2,6-dioxopiperidin-3-yl)isoindoline-1,3-dione (5MS-5-010).
e) 4-((8-(5-(2-((3,3-dimethyl-1-oxo-1,3-dihydroisobenzofuran-5-yl)amino)-4-(((S)-2-hydroxy-1-phenylethyl)amino)pyrimidin-5-yl)-1,3,4-oxadiazol-2-yl)octyl)amino)-2-(2,6-dioxopiperidin-3-yl)isoindoline-1,3-dione (MS-5-011).
   In one embodiment preferred compounds according to the present invention include:
a) 2-(2,6-dioxopiperidin-3-yl)-4-((3-(((2-(6-(4-ethyl-4H-1,2,4-triazol-3-yl)pyridin-2-yl)-6-((R)-2-methylpyrrolidin-1-yl)-1-oxo-2,3-dihydro-1H-pyrrolo[3,4-c]pyridin-4-yl)methyl)(methyl)amino)propyl)amino)isoindoline-1,3-dione (MS-6-007).
b) 2-(2,6-dioxopiperidin-3-yl)-4-((4-(((2-(6-(4-ethyl-4H-1,2,4-triazol-3-yl)pyridin-2-yl)-6-((R)-2-methylpyrrolidin-1-yl)-1-oxo-2,3-dihydro-1H-pyrrolo[3,4-c]pyridin-4-yl)methyl)(methyl)amino)butyl)amino)isoindoline-1,3-dione (MS-6-008).
c) 2-(2,6-dioxopiperidin-3-yl)-4-((6-(((2-(6-(4-ethyl-4H-1,2,4-triazol-3-yl)pyridin-2-yl)-6-((R)-2-methylpyrrolidin-1-yl)-1-oxo-2,3-dihydro-1H-pyrrolo[3,4-c]pyridin-4-yl)methyl)(methyl)amino)hexyl)amino)isoindoline-1,3-dione (MS-6-009).
d) 2-((2-(2,6-dioxopiperidin-3-yl)-1,3-dioxoisoindolin-4-yl)amino)-N-((2-(6-(4-ethyl-4H-1,2,4-triazol-3-yl)pyridin-2-yl)-6-((R)-2-methylpyrrolidin-1-yl)-1-oxo-2,3-dihydro-1H-pyrrolo[3,4-c]pyridin-4-yl)methyl)-N-methylacetamide (MS-6-024).

In some aspects, this disclosure provides a method of treating the HPK1-mediated diseases, the method including administering to a subject in need thereof with an HPK1-mediated disease one or more bivalent compounds including an HPK1 ligand conjugated to a degradation/disruption tag. The HPK1-mediated diseases may be a disease resulting from HPK1 amplification. The HPK1-mediated diseases can have elevated HPK1 enzymatic activity relative to a wild-type tissue of the same species and tissue type. Non-limiting examples of HPK1-mediated diseases or diseases whose clinical symptoms could be treated by HPK1 degraders/disruptors-mediated therapy include: all solid and liquid cancer, chronic infections that produce exhausted immune response, infection-mediated immune suppression, age-related decline in immune response, age-related decline in cognitive function and infertility.

Exemplary types of cancer that could prevented, or therapeutically treated by manipulation of HPK1 level by degraders/disruptors should include all solid and liquid cancers, including, but not limited to, cancers of the breast, respiratory tract, brain, reproductive organs, digestive tract, urinary tract, eye, liver, skin, head and neck, thyroid, parathyroid and their distant metastases. Examples of liquid cancers include lymphomas, sarcomas, and leukaemias. Listed below are the type of cancers that immunotherapy using HPK1 degraders/disruptors should be able to prevent or treat.

Examples of breast cancers include, but are not limited to, triple negative breast cancer, invasive ductal carcinoma, invasive lobular carcinoma, ductal carcinoma in situ, and lobular carcinoma in situ.

Examples of cancers of the respiratory tract include, but are not limited to, small-cell and non-small-cell lung carcinoma, as well as bronchial adenoma and pleuropulmonary blastoma.

Examples of brain cancers include, but are not limited to, brain stem and hypothalmic glioma, cerebellar and cerebral astrocytoma, glioblastoma, medulloblastoma, ependymoma, as well as neuroectodermal and pineal tumor.

Tumors of the male reproductive organs include, but are not limited to, prostate and testicular cancer.

Tumors of the female reproductive organs include, but are not limited to, endometrial, cervical, ovarian, vaginal, and vulvar cancer, as well as sarcoma of the uterus.

Examples of ovarian cancer include, but are not limited to, serous tumor, endometrioid tumor, mucinous cystadenocarcinoma, granulosa cell tumor, Sertoli-Leydig cell tumor and arrhenoblastoma.

Examples of cervical cancer include, but are not limited to, squamous cell carcinoma, adenocarcinoma, adenosquamous carcinoma, small cell carcinoma, neuroendocrine tumor, glassy cell carcinoma and villoglandular adenocarcinoma. Tumors of the digestive tract include, but are not limited to, anal, colon, colorectal, esophageal, gallbladder, gastric, pancreatic, rectal, small-intestine, and salivary gland cancers.

Examples of esophageal cancer include, but are not limited to, esophageal cell carcinomas and adenocarcinomas, as well as squamous cell carcinomas, leiomyosarcoma, malignant melanoma, rhabdomyosarcoma and lymphoma.

Examples of gastric cancer include, but are not limited to, intestinal type and diffuse type gastric adenocarcinoma.

Examples of pancreatic cancer include, but are not limited to, ductal adenocarcinoma, adenosquamous carcinomas and pancreatic endocrine tumors.

Example of tumors of the urinary tract include, but are not limited to, bladder, penile, kidney, renal pelvis, ureter, urethral and human papillary renal cancers.

Examples of kidney cancer include, but are not limited to, renal cell carcinoma, urothelial cell carcinoma, juxtaglomerular cell tumor (reninoma), angiomyolipoma, renal oncocytoma, Bellini duct carcinoma, clear-cell sarcoma of the kidney, mesoblastic nephroma and Wilms' tumor.

Examples of bladder cancer include, but are not limited to, transitional cell carcinoma, squamous cell carcinoma, adenocarcinoma, sarcoma and small cell carcinoma. Eye cancers include, but are not limited to, intraocular melanoma and retinoblastoma.

Examples of liver cancers include, but are not limited to, hepatocellular carcinoma (liver cell carcinomas with or without fibrolamellar variant), cholangiocarcinoma (intrahepatic bile duct carcinoma), and mixed hepatocellular cholangiocarcinoma.

Example of skin cancers include, but are not limited to, squamous cell carcinoma, Kaposi's sarcoma, malignant melanoma, Merkel cell skin cancer, and non-melanoma skin cancer.

Example of head-and-neck cancers include, but are not limited to, squamous cell cancer of the head and neck, laryngeal, hypopharyngeal, nasopharyngeal, oropharyngeal cancer, salivary gland cancer, lip and oral cavity cancer and squamous cell.

Example of lymphomas include, but are not limited to, AIDS-related lymphoma, non-Hodgkin's lymphoma, cutaneous T-cell lymphoma, Burkitt lymphoma, Hodgkin's disease, and lymphoma of the central nervous system.

Example of sarcomas include, but are not limited to, sarcoma of the soft tissue, osteosarcoma, malignant fibrous histiocytoma, lymphosarcoma, and rhabdomyosarcoma.

Example of leukemias include, but are not limited to, acute myeloid leukemia, acute lymphoblastic leukemia, chronic lymphocytic leukemia, chronic myelogenous leukemia, and hairy cell leukemia.

The HPK1 degraders/disruptors should be able to treat the above cancer types as stand alone agents or used as an agent in combination with existing standards of treatment therapy and other FDA-approved cancer therapy.

Therapeutic use of HPK1 extends to include diseases and therapies that are amenable to treatment by stimulation/augmentation of immune response, including the prolongation of immune responses during vaccination for immunizable diseases such as influenza and coronaviruses, including Covid 19. Also, because HPK1 is expressed at high level in two other anatomical locations—brain and testes—the HPK1 degraders/disruptors should be able to treat or prevent diseases related to brain and testes that were caused by HPK1 or could be treated by HPK1 degraders/disruptors. These potential diseases include, but are not limited to, Alzheimer's disease, age-related dementia and infertility, regardless whether these possible diseases were caused by HPK1 or by other etiological causes.

Therapeutic use of HPK1 further extends to include therapies involving ex vivo treatment of immune cells, including, but not limited to, all T cell subsets, genetically engineered T cells, Chimeric Antigen Receptor (CAR) T cells, tumor infiltrating lymphocytes, dendritic cells, macrophage, mast cells, granulocytes (include basophils, eosinophils, and neutrophils), natural killer cells, NK T cells and B cells. Such cells would be therapeutically treated by HPK1 degraders and then re-introduced back to the patient being treated for conditions that would benefit from reduction in HPK1 expression. The sources of cells for such ex vivo treatment include, but are not limited to, the autologous bone marrow cells from the patient him/herself, or from the patient's frozen banked cord blood stem cells, peripheral blood or bone marrow stem cells from MHC-matched or MHC-mismatched donors.

Treating patients by administering specific immune cells that had been treated with HPK1 degraders offers many added advantages over in vivo use. By treating specific immune cells type with HPK1 degraders ex vivo, it is possible to specifically target the immune cell type that would receive the benefit of having the endogenous HPK1 level reduced by HPK1 degraders while sparing the HPK1 expression level in other immune cell types that are not involved in the disease condition.

This therapeutic approach would provide cell type-specific targeting of immune cells in a way that is not possible with the use of HPK1 degrader in the in vivo setting. Thus, the ex vivo approach would likely limit potential toxicity that may result from reduction of HPK1 level in immune cell types that do not benefit from a reduction in HPK1 levels.

Furthermore, by administering HPK1 degraders in the ex vivo cell setting, the risk of patients experiencing the toxicity or undesirable outcome that might occur should the HPK1 degraders were to be administered systemically would be eliminated.

It is known that HPK1 is also expressed in non-hematopoietically-derived tissues such as the brain and testes. Because of this tissue-specific expression pattern of HPK1, HPK1 degraders might be able to treat or prevent diseases related to the brain and testes that were caused by HPK1.

These potential treatments include, but are not limited to, treatment of Alzheimer's disease, age-related dementia and infertility, irrespective to whether these possible diseases were caused by HPK1 or by other etiological causes.

The use of HPK1 expression status of the tumor as the biomarker would enable stratification of patients into appropriate therapeutic groups that would receive HPK1 degraders in vivo or ex vivo, based on HPK1 expression in the tumors.

Furthermore, using HPK1 degraders in an ex vivo setting offers additional advantages over gene-editing approaches such as CRISPR in that it allows therapeutic use of HPK1 degraders as a non-permanent treatment that allows a therapeutic regimen to be adjusted temporally through dosing levels and through alteration of the administration schedule.

In addition, HPK1 degraders could be used in settings whereby stimulation/augmentation of the immune response is required, or when the prolongation of immune responses is needed. Improving immune response to vaccination is one of the settings in which HPK1 degraders could be used therapeutically. HPK1 degraders could also be used to enhance the antigen presentation capability of dendritic cell-based cancer vaccines.

Other utilities of HPK1 degraders include treatment of dendritic cells with HPK1 degraders to increase resistance to maturation-induced apoptosis, thus increasing the yield of dendritic cell production.

In an embodiment, HPK1 degraders of the present invention may be employed in combination with treatments using checkpoint inhibitors, including, but not limited to anti-programmed cell death protein (anti-PD-1) and anti-programmed death ligand-1 (anti-PD-L1). Examples of anti-PD-1 and anti-PD-L1 agents include monoclonal antibodies that target either PD-1 or PD-L1. Such antibodies include, but are not limited to pembrolizumab (Keytruda), nivolumab (Opdivo), and cemiplimab (Libtayo) (PD-1 inhibitors); and atezolizumab (Tecentriq), avelumab (Bavencio), and durvalumab (Imfinzi) (PD-L1 inhibitors). Use of such anti-PD1 and/or anti-PD-L1 agents in immunotherapy, particularly cancer immunotherapy, may be enhanced by concomitant therapy with HPK1 degraders of the present invention. Such combination therapy of anti-PD-1 agents with HPK1 degraders of the present invention is particularly useful in the treatment of melanoma, lung cancer, renal cell carcinoma, Hodgkin lymphoma, head and neck cancer, colon cancer and liver cancer. Such combination therapy of anti-PD-L1 agents with HPK1 degraders of the present invention are particularly useful in the treatment of non-small cell lung carcinoma, multiple myeloma, urothelial cancer and head and neck cancer. (Hernandez 2018).

Similar combination therapy may employ HPK1 degraders of the present invention with an anti-CTLA-4 (cytotoxic T-lymphocyte-associated protein 4) agent, such as the monoclonal antibody ilimumab, particularly for the treatment of melanoma, lung cancer, renal cell carcinoma, glioblastoma, hepatocellular carcinoma large B cell lymphoma, Hodgkin lymphoma, head and neck cancer, colon cancer and liver cancer.

In an embodiment, the HPK1 degraders of the present invention may be used in the treatment of tumor types having elevated expression of cyclooxygenase-2 (COX-2). COX-2 elevation leads to over production of prostaglandin E2 (PGE2). PGE2 made by these tumors is known to inhibit the anti-tumor immune response. T cells lacking HPK1 are resistant to PGE2-mediated inhibition. (Alzabin 2010). Cancer types known to have high expression levels of COX-2 include, but not are not limited to colon cancer, lung cancer, sarcoma and breast cancer.

In any of the above described methods, HPK1 degraders can be MS-5-006, MS-5-007, MS-5-008, MS-5-009, MS-5-010, MS-5-011, MS-5-015, MS-5-016, MS-5-017, MS-5-018, MS-5-019, MS-5-021, MS-5-022, MS-5-024, MS-5-026, MS-5-027, MS-5-028, MS-5-029, MS-5-030, MS-5-033, MS-5-034, MS-5-035, MS-5-036, MS-5-037, MS-5-038, MS-5-039, MS-5-040, MS-5-041, MS-5-042, MS-5-043, MS-5-044, MS-5-045, MS-5-046, MS-6-003, MS-6-004, MS-6-005, MS-6-006, MS-6-007, MS-6-008, MS-6-009, MS-6-013, MS-6-014, MS-6-015, MS-6-016, MS-6-017, MS-6-018, MS-6-019, MS-6-020, MS-6-021, MS-6-022, MS-6-023, MS-6-024, MS-6-025, MS-6-026, MS-6-027, MS-6-028, MS-6-029, MS-6-037, MS-6-038, MS-6-039, MS-6-040, MS-6-041, MS-6-043, MS-6-044, MS-6-045, MS-6-046, MS-6-047, MS-6-048, MS-6-049, MS-6-050, MS-6-051, MS-6-052, MS-6-053, MS-6-054, MS-6-055, MS-6-058, MS-6-059, MS-6-060, MS-6-061.

In some aspects of the disclosed methods, the bivalent compounds can be administered by any of several routes of administration including, e.g., orally, parenterally, intradermally, subcutaneously, topically, and/or rectally.

Any of the above-described methods can further include treating the subject with one or more additional therapeutic regimens for treating cancer. The one or more additional therapeutic regimens for treating cancer can be, e.g., one or more of surgery, chemotherapy, radiation therapy, hormone therapy, or immunotherapy.

This disclosure additionally provides a method for identifying a bivalent compound which mediates degradation/disruption of HPK1, the method including providing a heterobifunctional test compound including a HPK1 ligand conjugated to a degradation/disruption tag, contacting the heterobifunctional test compound with a cell (e.g., a cancer cell such as a HPK1-mediated cancer cell) including a ubiquitin ligase and HPK1.

As used herein, the terms “about” and “approximately” are defined as being within plus or minus 10% of a given value or state, preferably within plus or minus 5% of said value or state. Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF DRAWINGS

FIGS. 1-3 show the effect of synthesized compounds on TCR-induced IL-2 production by Jurkat T cells and the effect of select compound on the HPK1 expression level in Jurkat T cells.

FIG. 4 depicts a reaction scheme for the synthesis of MS-5-006.

FIG. 5 depicts a reaction scheme for the synthesis of MS-5-007.

FIG. 6 depicts a reaction scheme for the synthesis of MS-5-016.

FIG. 7 depicts a reaction scheme for the synthesis of MS-5-017.

FIG. 8 depicts a reaction scheme for the synthesis of MS-5-019.

FIG. 9 depicts a reaction scheme for the synthesis of MS-5-027.

FIG. 10 depicts a reaction scheme for the synthesis of MS-5-028.

FIG. 11 depicts a reaction scheme for the synthesis of MS-5-029.

FIG. 12 depicts a reaction scheme for the synthesis of MS-5-035.

FIG. 13 depicts a reaction scheme for the synthesis of MS-5-039.

FIG. 14 depicts a reaction scheme for the synthesis of MS-5-041.

FIG. 15 depicts a reaction scheme for the synthesis of MS-6-017.

FIG. 16 depicts a reaction scheme for the synthesis of MS-6-027.

FIG. 17 depicts a reaction scheme for the synthesis of MS-6-028.

FIG. 18 depicts a reaction scheme for the synthesis of MS-6-029.

FIG. 19 depicts a reaction scheme for the synthesis of MS-6-046.

### DETAILED DESCRIPTION

The present disclosure is based, in part, on the discovery that novel heterobifunctional molecules which degrade HPK1, HPK1 fusion proteins, and/or HPK1 mutant proteins are useful in the treatment of HPK1-mediated diseases.

Non-limiting examples of HPK1-mediated diseases or diseases whose clinical symptoms could be treated by HPK1 degraders/disruptors-mediated therapy include: all solid and liquid cancer, chronic infections that produce exhausted immune response, infection-mediated immune suppression, age-related decline in immune response, age-related decline in cognitive function and infertility

Exemplary type of cancers that could be prevented, or therapeutically treated by manipulation of HPK1 level by degraders/disruptors should include all solid and liquid cancers, including, but not limited to, cancers of the breast, respiratory tract, brain, reproductive organs, digestive tract, urinary tract, eye, liver, skin, head and neck, thyroid, parathyroid and their distant metastases. Examples of liquid cancers include lymphomas, sarcomas, and leukaemias. Listed below are the type of cancers that immunotherapy using HPK1 degraders/disruptors should be able to prevent or treat.

Examples of breast cancers include, but are not limited to, triple negative breast cancer, invasive ductal carcinoma, invasive lobular carcinoma, ductal carcinoma in situ, and lobular carcinoma in situ.

Examples of cancers of the respiratory tract include, but are not limited to, small-cell and non-small-cell lung carcinoma, as well as bronchial adenoma and pleuropulmonary blastoma.

Examples of brain cancers include, but are not limited to, brain stem and hypothalmic glioma, cerebellar and cerebral astrocytoma, glioblastoma, medulloblastoma, ependymoma, as well as neuroectodermal and pineal tumor.

Tumors of the male reproductive organs include, but are not limited to, prostate and testicular cancer.

Tumors of the female reproductive organs include, but are not limited to, endometrial, cervical, ovarian, vaginal, and vulvar cancer, as well as sarcoma of the uterus.

Examples of ovarian cancer include, but are not limited to, serous tumor, endometrioid tumor, mucinous cystadenocarcinoma, granulosa cell tumor, Sertoli-Leydig cell tumor and arrhenoblastoma.

Examples of cervical cancer include, but are not limited to, squamous cell carcinoma, adenocarcinoma, adenosquamous carcinoma, small cell carcinoma, neuroendocrine tumor, glassy cell carcinoma and villoglandular adenocarcinoma. Tumors of the digestive tract include, but are not limited to, anal, colon, colorectal, esophageal, gallbladder, gastric, pancreatic, rectal, small-intestine, and salivary gland cancers.

Examples of esophageal cancer include, but are not limited to, esophageal cell carcinomas and adenocarcinomas, as well as squamous cell carcinomas, leiomyosarcoma, malignant melanoma, rhabdomyosarcoma and lymphoma.

Examples of gastric cancer include, but are not limited to, intestinal type and diffuse type gastric adenocarcinoma.

Examples of pancreatic cancer include, but are not limited to, ductal adenocarcinoma, adenosquamous carcinomas and pancreatic endocrine tumors.

Example of tumors of the urinary tract include, but are not limited to, bladder, penile, kidney, renal pelvis, ureter, urethral and human papillary renal cancers.

Examples of kidney cancer include, but are not limited to, renal cell carcinoma, urothelial cell carcinoma, juxtaglomerular cell tumor (reninoma), angiomyolipoma, renal oncocytoma, Bellini duct carcinoma, clear-cell sarcoma of the kidney, mesoblastic nephroma and Wilms' tumor.

Examples of bladder cancer include, but are not limited to, transitional cell carcinoma, squamous cell carcinoma, adenocarcinoma, sarcoma and small cell carcinoma. Eye cancers include, but are not limited to, intraocular melanoma and retinoblastoma.

Examples of liver cancers include, but are not limited to, hepatocellular carcinoma (liver cell carcinomas with or without fibrolamellar variant), cholangiocarcinoma (intrahepatic bile duct carcinoma), and mixed hepatocellular cholangiocarcinoma.

Example of skin cancers include, but are not limited to, squamous cell carcinoma, Kaposi's sarcoma, malignant melanoma, Merkel cell skin cancer, and non-melanoma skin cancer.

Example of head-and-neck cancers include, but are not limited to, squamous cell cancer of the head and neck, laryngeal, hypopharyngeal, nasopharyngeal, oropharyngeal cancer, salivary gland cancer, lip and oral cavity cancer and squamous cell.

Example of lymphomas include, but are not limited to, AIDS-related lymphoma, non-Hodgkin's lymphoma, cutaneous T-cell lymphoma, Burkitt lymphoma, Hodgkin's disease, and lymphoma of the central nervous system.

Example of sarcomas include, but are not limited to, sarcoma of the soft tissue, osteosarcoma, malignant fibrous histiocytoma, lymphosarcoma, and rhabdomyosarcoma.

Example of leukemias include, but are not limited to, acute myeloid leukemia, acute lymphoblastic leukemia, chronic lymphocytic leukemia, chronic myelogenous leukemia, and hairy cell leukemia.

The HPK1 degraders/disruptors should be able to treat the above cancer type as stand alone agent or used as agent in combination with existing standard of treatment therapy and other FDA-approved cancer therapy.

Therapeutic uses of HPK1 include diseases and therapies that are amenable to treatment by stimulation/augmentation of immune response, including the prolongation of immune responses during vaccination for immunizable diseases. Also, because HPK1 is expressed at high level in two other anatomical locations—brain and testes—the HPK1 degraders/disruptors should be able to treat or prevent diseases related to brain and testes that were caused by HPK1 or could be treated by HPK1 degraders/disruptors. These potential diseases include, but is not limited to, Alzheimer's disease, age-related dementia and infertility, regardless whether these possible diseases were caused by HPK1 or by other etiological causes.

Successful strategies for selective degradation/disruption of the target protein induced by a bifunctional molecule include recruiting an E3 ubiquitin ligase and mimicking protein misfolding with a hydrophobic tag (Buckley and Crews, 2014). PROTACs (PROteolysis TArgeting Chimeras) are bivalent molecules with one moiety that binds an E3 ubiquitin ligase and another moiety that binds the protein target of interest (Buckley and Crews, 2014). The induced proximity leads to selective ubiquitination of the target followed by its degradation at the proteasome. Several types of high affinity small-molecule E3 ligase ligands have been identified/developed: They include (1) immunomodulatory drugs (IMiDs) such as thalidomide and pomalidomide, which bind cereblon (CRBN or CRL4^{CRBN}), a component of a cullin-RING ubiquitin ligase (CRL) complex (Bondeson et al., 2015; Chamberlain et al., 2014; Fischer et al., 2014; Ito et al., 2010; Winter et al., 2015); (2) VHL-1, a hydroxyproline-containing ligand, which binds van Hippel-Lindau protein (VHL or CRL2^{VHL}), a component of another CRL complex (Bondeson et al., 2015; Buckley et al., 2012a; Buckley et al., 2012b; Galdeano et al., 2014; Zengerle et al., 2015); (3) compound 7, which selectively binds KEAP1, a component of a CRL3 complex (Davies et al., 2016); (4) AMG232, which selectively binds MDM2, a heterodimeric RING E3 ligase (Sun et al., 2014); and (5) LCL161, which selectively binds IAP, a homodimeric RING E3 ligase (Ohoka et al., 2017; Okuhira et al, 2011; Shibata et al., 2017). The degrader technology has been successfully applied to degradation of multiple targets (Bondeson et al., 2015; Buckley et al., 2015; Lai et al., 2016; Lu et al., 2015; Winter et al., 2015; Zengerle et al., 2015), but not to degradation of HPK1. In addition, a hydrophobic tagging approach, which utilizes a bulky and hydrophobic adamantyl group, has been developed to mimic protein misfolding, leading to the degradation of the target protein by proteasome (Buckley and Crews, 2014). This approach has also been successfully applied to selective degradation of the pseudokinase Her3 (Xie et al., 2014), but not to degradation of HPK1 proteins.

As discussed in the following examples, this disclosure provides specific examples of novel HPK1 degraders/disruptors, and examined the effect of exemplary degraders/disruptors on reducing HPK1 protein levels, inhibiting/disrupting HPK1 activity and increasing the TCR-induced IL-2 production by Jurkat T cells. The results indicated that these novel compounds can be beneficial in treating cancer. Exemplary type of cancers that could be prevented, or therapeutically treated by manipulation of HPK1 level by degraders/disruptors should include all solid and liquid cancers, including, but not limited to, cancers of the breast, respiratory tract, brain, reproductive organs, digestive tract, urinary tract, eye, liver, skin, head and neck, thyroid, parathyroid and their distant metastases. Examples of liquid cancers include lymphomas, sarcomas, and leukaemias. Listed below are the type of cancers that immunotherapy using HPK1 degraders/disruptors should be able to prevent or treat as mentioned above.

Current compounds targeting HPK1 generally focus on inhibition of its catalytic activity. In the present disclosure a different approach was taken: to develop compounds that directly and selectively target not only the catalytic function of HPK1, but also its protein level in cells. Strategies for inducing protein degradation include recruiting E3 ubiquitin ligases, mimicking protein misfolding with hydrophobic tags, and inhibiting chaperones. For example, a thalidomide-JQ1 bivalent compound has been used to hijack the cereblon E3 ligase, inducing highly selective BET protein degradation in vitro and in vivo and resulting in a demonstrated delay in leukemia progression in mice (Winter et al., 2015). Similarly, BET protein degradation has also been induced via another E3 ligase, VHL (Zengerle et al., 2015). Partial degradation of the Her3 protein has been induced using an adamantane-modified compound (Xie et al., 2014). Such an approach, based on the use of bivalent molecules, permits more flexible regulation of protein levels in vitro and in vivo compared with techniques such as gene knockout or knockdown via RNA interference. Unlike gene knockout or knockdown, this chemical approach provides an opportunity to study dose and time dependency in a disease model by varying the concentrations and frequencies of administration of the relevant compound.

This disclosure includes all stereoisomers, geometric isomers, tautomers and isotopes of the structures depicted and compounds named herein. This disclosure also includes compounds described herein, regardless of how they are prepared, e.g., synthetically, through biological process (e.g., metabolism or enzyme conversion), or a combination thereof.

This disclosure includes pharmaceutically acceptable salts of the structures depicted and compounds named herein.

One or more constituent atoms of the compounds presented herein can be replaced or substituted with isotopes of the atoms in natural or non-natural abundance. In some embodiments, the compound includes at least one deuterium atom In some embodiments, the compound includes two or more deuterium atoms. In some embodiments, the compound includes 1-2, 1-3, 1-4, 1-5, or 1-6 deuterium atoms. In some embodiments, all of the hydrogen atoms m a compound can be replaced or substituted by deuterium atoms. In some embodiments, the compound includes at least one fluorine atom In some embodiments, the compound includes two or more fluorine atoms. In some embodiments, the compound includes 1-2, 1-3, 1-4, 1-5, or 1-6 fluorine atoms. In some embodiments, all of the hydrogen atoms in a compound can be replaced or substituted by fluorine atoms.

### Degraders

In some aspects, the present disclosure provides bivalent compounds, also referred to herein as degarders, comprising a HPK1 ligand (or targeting moiety) conjugated to a degradation tag. Linkage of the HPK1 ligand to the degradation tag can be direct, or indirect via a linker.

As used herein, the terms “protein arginine methyltransferase 5 (HPK1) ligand” or “HPK1 ligand” or “HPK1 targeting moiety” are to be construed broadly, and encompass a wide variety of molecules ranging from small molecules to large proteins that associate with or bind to HPK1. The HPK1 ligand or targeting moiety can be, for example, a small molecule compound (i.e., a molecule of molecular weight less than about 1.5 kilodaltons (kDa)), a peptide or polypeptide, nucleic acid or oligonucleotide, carbohydrate such as oligosaccharides, or an antibody or fragment thereof.

The HPK1 ligand or targeting moiety can be derived from a HPK1 inhibitor (e.g., sutent and analogs thereof), which is capable of interfering with the enzymatic activity of HPK1. As used herein, an “inhibitor” refers to an agent that restrains, retards, or otherwise causes inhibition of a physiological, chemical or enzymatic action or function. As used herein an inhibitor causes a decrease in enzyme activity of at least 5%. An inhibitor can also or alternatively refer to a drug, compound, or agent that prevents or reduces the expression, transcription, or translation of a gene or protein. An inhibitor can reduce or prevent the function of a protein, e.g., by binding to or activating/inactivating another protein or receptor.

Exemplary HPK1 ligands include, but are not limited to, the compounds listed below:

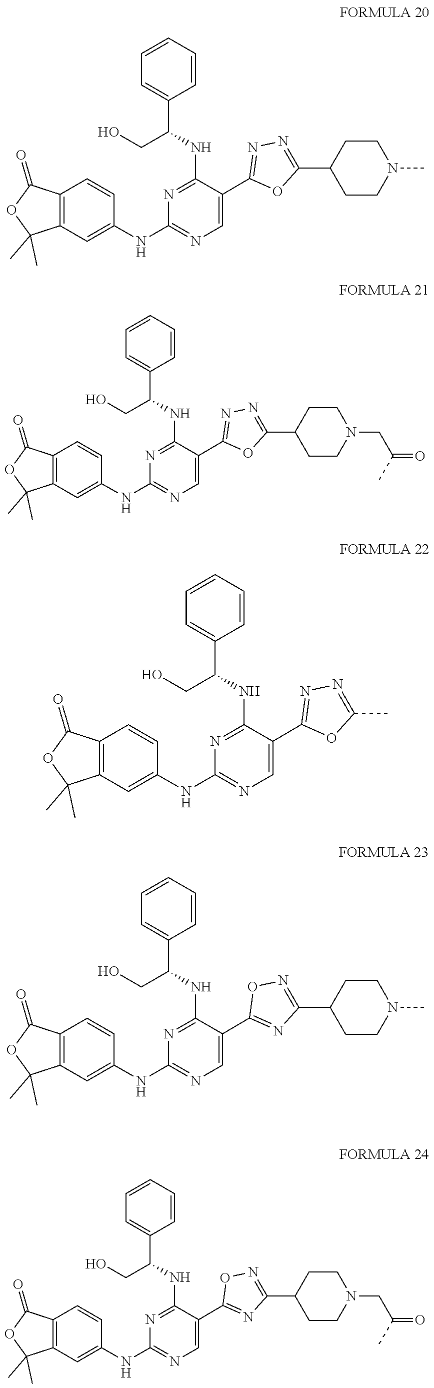

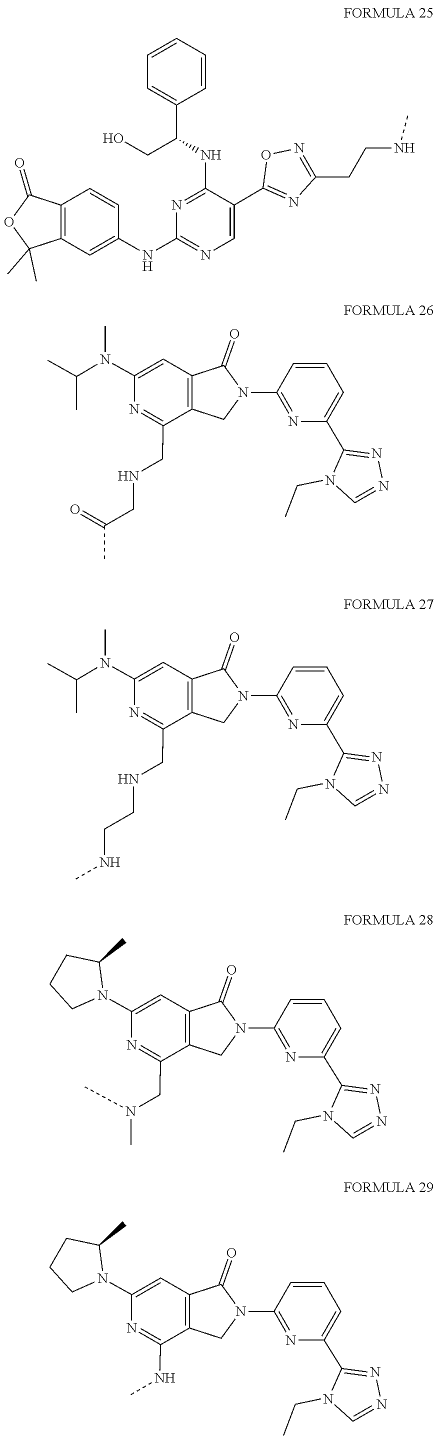

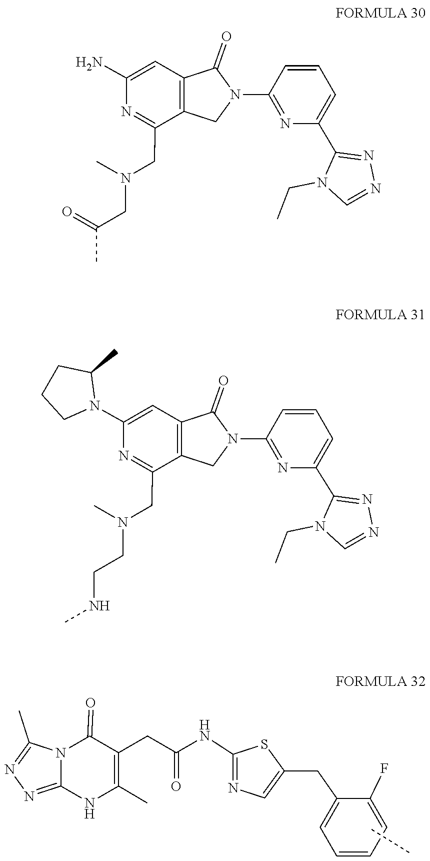

As used herein, the term “degradation/disruption tag” refers to a compound, which associates with or binds to a ubiquitin ligase for recruitment of the corresponding ubiquitination machinery to HIPK1 or induces HIPK1 protein misfolding and subsequent degradation at the proteasome or loss of function.

In some aspects, the degradation/disruption tags of the present disclosure include, e.g., thalidomide, pomalidomide, lenalidomide, VHL-1, adamantane, 1-((4,4,5,5,5-pentafluoropentyl)sulfinyl)nonane, nutlin-3a, RG7112, RG7338, AMG232, AA-115, bestatin, MV-1, LCL161, and/or analogs thereof.

As used herein, a “linker” is a bond, molecule, or group of molecules that binds two separate entities to one another. Linkers can provide for optimal spacing of the two entities. The term “linker” in some aspects refers to any agent or molecule that bridges the HIPK1 ligand to the degradation/disruption tag. One of ordinary skill in the art recognizes that sites on the HPK1 ligand or the degradation/disruption tag, which are not necessary for the function of the degraders of the present disclosure, are ideal sites for attaching a linker, provided that the linker, once attached to the conjugate of the present disclosure, does not interfere with the function of the degrader, i.e., its ability to target HPK1 and its ability to recruit a ubiquitin ligase.

The length of the linker of the bivalent compound can be adjusted to minimize the molecular weight of the disruptors/degraders and avoid any potential clash of the HPK1 ligand or targeting moiety with either the ubiquitin ligase or the induction of HPK1 misfolding by the hydrophobic tag at the same time.

In some aspects, the degradation/disruption tags of the present disclosure include, for example, thalidomide, pomalidomide, lenalidomide, VHL-1, adamantane, 1-((4,4,5,5,5-pentafluoropentyl)sulfinyl)nonane, nutlin-3a, RG7112, RG7338, AMG 232, AA-115, bestatin, MV-1, LCL161, and analogs thereof. The degradation/disruption tags can be attached to any portion of the structure of a HPK1 ligand or targeting moiety (e.g., sutent) with linkers of different types and lengths in order to generate effective bivalent compounds. In particular, attaching VHL1, pomalidomide, or LCL161 to any portion of the molecule can recruit the E3 ligase to HPK1.

The bivalent compounds disclosed herein can increasing the TCR-induced IL-2 production by Jurkat T cells.
Additional bivalent compounds (i.e., HPK1 degraders/disruptors) can be developed using the principles and methods disclosed herein. For example, other linkers, degradation tags, and HPK1 binding/inhibiting moieties can be synthesized and tested. Non-limiting examples of HPK1 disruptors/degraders (e.g., bivalent compounds) are shown in Table 1 (below). The left portion of each HPK1 disruptors/degrader compound as shown binds to HPK1 (as sutent (sunitinib) do), and the right portion of each compound recruits for the ubiquitination machinery to HPK1, which induces the poly-ubiquitination and degradation of HPK1 at the proteasome.

More specifically, the present disclosure provides a bivalent compound including a HPK1 ligand conjugated to a degradation/disruption tag.

In some aspects, the HPK1 degraders/disruptors have the form “PI-linker-EL”, as shown below:

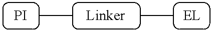

wherein PI (protein of interest) comprises an HPK1 ligand (e.g., an HPK1 inhibitor) and EL (E3 ligase) comprises a degradation/disruption tag (e.g., E3 ligase ligand). Exemplary HPK1 ligands (PI), exemplary degradation/disruption tags (EL), and exemplary linkers (Linker) are illustrated below:

### HPK1 Ligands

In an embodiment, (HPK1) ligands include a moiety according to FORMULA 1:

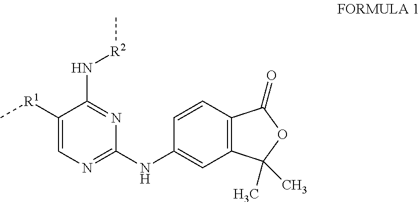

wherein
the “Linker” moiety of the bivalent compound is attached independently to R¹ or R² (indicated by dotted line);
When the “Linker” moiety of the bivalent compound is attached to R²;
R¹ is, at each occurence, independently selected from hydrogen, halogen, oxo, CN, NO₂, OR⁴, SR⁴, NR⁴R⁵, C(O)R⁴, C(O)OR⁴, C(O)NR⁴R⁵, S(O)R⁴, S(O)₂R⁴, S(O)₂NR⁴R⁵, NR⁶C(O)OR⁴, NR⁶C(O)R⁴, NR⁶C(O)NR⁴R⁵, NR⁶S(O)R⁴, NR⁶S(O)₂R⁴, NR⁶S(O)₂NR⁴R⁵, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl; wherein

R⁴, R⁵, and R⁶ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl; or

R⁴ and R⁵, R⁴ and R⁶, R⁵ and R⁶ together with the atom to which they are connected form an optionally substituted 3-20 membered cycloalkyl or heterocyclyl ring; and pharmaceutically acceptable salts thereof.

When the “Linker” moiety of the bivalent compound is attached to R¹;
R¹ is, at each occurence, independently selected from OR⁴, SR⁴, NR⁴R⁵, C(O)R⁴, C(O)OR⁴, C(O)NR⁴R⁵, S(O)R⁴, S(O)₂R⁴, S(O)₂NR⁴R⁵, NR⁶C(O)OR⁴, NR⁶C(O)R⁴, NR⁶C(O)NR⁴R⁵, NR⁶S(O)R⁴, NR⁶S(O)₂R⁴, NR⁶S(O)₂NR⁴R⁵, C₁-C₈ alkylene, optionally substituted C₁-C₈ alkylene, optionally substituted C₂-C₈ alkenylene, optionally substituted C₂-C₈ alkynylene, optionally substituted C₁-C₈ alkoxylene, optionally substituted C₁-C₈alkoxyC₁-C₈alkylene, optionally substituted C₁-C₈alkylaminoC₁-C₈alkylene, optionally substituted 3-8 membered cycloalkylene, optionally substituted C₃-C₈ cycloalkoxylene, optionally substituted 3-8 membered heterocyclylene, optionally substituted arylene, and optionally substituted heteroarylene; wherein

R⁴ is null, or a bivalent moiety selected from optionally substituted C₁-C₈ alkylene, optionally substituted C₂-C₈ alkenylene, optionally substituted C₂-C₈ alkynylene, optionally substituted C₃-C₈ cycloalkylene, optionally substituted 3-8 membered heterocyclylene, optionally substituted arylene, and optionally substituted heteroarylene;

R⁵ and R⁶ are independently selected from optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₈ cycloalkyl, optionally substituted 3-8 membered heterocyclyl, optionally substituted C₂-C₈ alkenyl, optionally substituted C₂-C₈ alkynyl, optionally substituted aryl, and optionally substituted heteroaryl; or

R⁴ and R⁵, R⁴ and R⁶, R⁵ and R⁶ together with the atom to which they are connected form a 3-20 membered cycloalkyl or heterocyclyl ring;

and pharmaceutically acceptable salts thereof.
When the “Linker” moiety of the bivalent compound is attached to R¹;
R² is, at each occurence, independently selected from null, hydrogen, C(O)R⁷, C(O)OR⁷, C(O)NR⁷R⁸, S(O)R⁷, S(O)₂R⁷, S(O)₂NR⁷R⁸, NR⁹C(O)OR⁷, NR⁹C(O)R⁷, NR⁹C(O)NR⁷R⁸, NR⁹S(O)R⁷, NR⁹S(O)₂R⁷, NR⁹S(O)₂NR⁷R⁸, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl; wherein
R⁷, R⁸, and R⁹ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl; or

R⁷ and R⁸, R⁷ and R⁹, R⁸ and R⁹ together with the atom to which they are connected form an optionally substituted 3-20 membered cycloalkyl or heterocyclyl ring; and pharmaceutically acceptable salts thereof.

When the “Linker” moiety of the bivalent compound is attached to R²;
R² is, at each occurence, independently selected from C(O)R⁷, C(O)OR⁷, C(O)NR⁷R⁸, S(O)R⁷, S(O)₂R⁷, S(O)₂NR⁷R⁸, NR⁹C(O)OR⁷, NR⁹C(O)R⁷, NR⁹C(O)NR⁷R⁸, NR⁹S(O)R⁷, NR⁹S(O)₂R⁷, NR⁹S(O)₂NR⁷R⁸, optionally substituted C₁-C₈ alkylene, optionally substituted C₂-C₈ alkenylene, optionally substituted C₂-C₈ alkynylene, optionally substituted C₁-C₈ alkoxylene, optionally substituted C₁-C₈alkoxyC₁-C₈alkylene, optionally substituted C₁-C₈alkylaminoC₁-C₈alkylene, optionally substituted 3-8 membered cycloalkylene, optionally substituted C₃-C₈ cycloalkoxylene, optionally substituted 3-8 membered heterocyclylene, optionally substituted arylene, and optionally substituted heteroarylene; wherein

R⁷ is null, or a bivalent moiety selected from optionally substituted C₁-C₈ alkylene, optionally substituted C₂-C₈ alkenylene, optionally substituted C₂-C₈ alkynylene, optionally substituted C₃-C₈ cycloalkylene, optionally substituted 3-8 membered heterocyclylene, optionally substituted arylene, and optionally substituted heteroarylene;

R⁸ and R⁹ are independently selected from optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₈ cycloalkyl, optionally substituted 3-8 membered heterocyclyl, optionally substituted C₂-C₈ alkenyl, optionally substituted C₂-C₈ alkynyl, optionally substituted aryl, and optionally substituted heteroaryl; or

R⁷ and R⁸, R⁷ and R⁹, R⁸ and R⁹ together with the atom to which they are connected form a 3-20 membered cycloalkyl or heterocyclyl ring;

and pharmaceutically acceptable salts thereof.
In another embodiment, (HPK1) ligands include a moiety according to FORMULA 2:

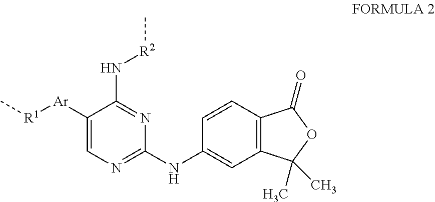

wherein
the “Linker” moiety of the bivalent compound is attached independently to R¹ or R² (indicated by dotted line);
The definitions of, R¹ and R², are the same as for FORMULA 1.
Ar is, at each occurence, independently selected from null, aryl or heteroaryl, each of which is substituted with R¹ and optionally substituted with one or more substituents independently selected from hydrogen, halogen, oxo, CN, NO₂, OR¹², SR¹², NR¹²R¹³, OCOR¹², OCO₂R¹², OCONR¹²R¹³, COR¹², CO₂R¹², CONR¹²R¹³, SOR¹², SO₂R¹², SO₂NR¹²R¹³, NR¹⁴CO₂R¹², NR¹⁴COR¹², NR¹⁴C(O)NR¹²R¹³, NR¹⁴SOR¹², NR¹⁴SO₂R¹², NR¹⁴SO₂NR¹²R¹³, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted 3-8 membered cycloalkyl, optionally substituted C₃-C₈ cycloalkoxy, optionally substituted 4-8 membered heterocyclyl, optionally substituted aryl, and optionally substituted heteroaryl; wherein

R¹², R¹³, and R¹⁴ are independently selected from hydrogen, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted 3-8 membered cycloalkyl, optionally substituted C₃-C₈ cycloalkoxy, optionally substituted 4-8 membered heterocyclyl, optionally substituted aryl, and optionally substituted heteroaryl; or

R¹² and R¹³, R¹² and R¹⁴, R¹³ and R¹⁴ together with the atom to which they are connected form a 4-20 membered heterocyclyl ring; and pharmaceutically acceptable salts thereof.

In an embodiment, (HPK1) ligands include a moiety according to FORMULA 2A:

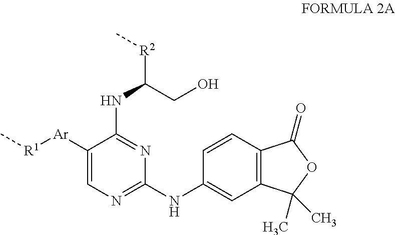

wherein
the “Linker” moiety of the bivalent compound is attached independently to R¹ or R² (indicated by dotted line);
The definitions of, R¹, R² and Ar are the same as for FORMULA 2.
In another embodiment, (HPK1) ligands include a moiety according to FORMULA 2B:

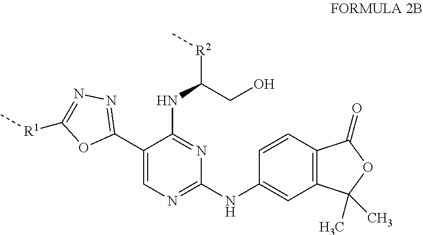

wherein
the “Linker” moiety of the bivalent compound is attached independently to R¹ or R² (indicated by dotted line);
The definitions of, R¹ and R² are the same as for FORMULA 2.
In another embodiment, (HPK1) ligands include a moiety according to FORMULA 2C:

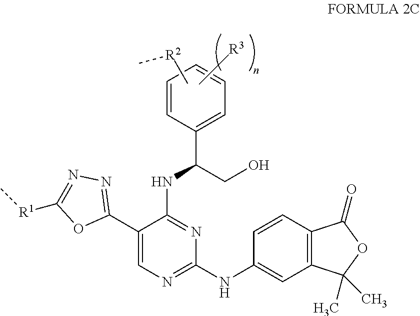

### Wherein

the “Linker” moiety of the bivalent compound is attached independently to R¹ or R² (indicated by dotted line);
The definitions of, R¹ and R² are the same as for FORMULA 2;
R³ is, at each occurence, independently selected from hydrogen, halogen, oxo, CN, NO₂, OR⁵, SR¹⁵, NR¹⁵R¹⁶C(O)R¹⁶, C(O)OR¹⁵, C(O)NR¹⁵R¹⁶, S(O)R¹, S(O)₂R¹⁵, S(O)₂NR¹⁵R¹⁶, NR¹⁷C(O)OR¹⁵, NR⁶C(O)R¹⁵, NR¹⁷C(O)NR¹⁵R¹⁶, NR¹⁷S(O)R¹⁵, NR¹⁷S(O)₂R¹⁵, NR¹⁷S(O)₂NR¹⁵R¹⁶, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl; wherein

R¹⁵, R¹⁶, and R¹⁷ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl; or

R¹⁵ and R¹⁶, R¹⁵ and R¹⁷, R¹⁶ and R¹⁷ together with the atom to which they are connected form an optionally substituted 3-20 membered cycloalkyl or heterocyclyl ring;

n is independently selected from 0, 1, 2, 3 and 4;
and pharmaceutically acceptable salts thereof.
In another embodiment, (HPK1) ligands include a moiety according to FORMULA 3:

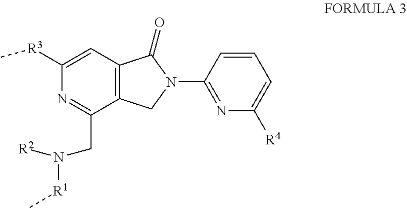

wherein
the “Linker” moiety of the bivalent compound is attached independently to R¹ or R³ (indicated by dotted line);
When the “Linker” moiety of the bivalent compound is attached to R³;
R¹ and R² are, at each occurence, independently selected from null, hydrogen, C(O)R¹⁰, C(O)OR¹⁰, C(O)NR¹⁰R¹¹, S(O)R¹⁰, S(O)₂R¹⁰, S(O)₂NR¹⁰R¹¹, NR¹²C(O)OR¹⁰, NR¹²C(O)R¹⁰, NR¹²C(O)NR¹⁰R¹¹, NR¹²S(O)R¹⁰, NR¹²S(O)₂R¹°, NR¹²S(O)₂NR¹⁰R¹¹, optionally substituted C₁—C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl; wherein
R¹⁰, R¹¹, and R¹² are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl; or

R¹⁰ and R¹¹, R¹⁰ and R¹², R¹¹ and R¹² together with the atom to which they are connected form an optionally substituted 3-20 membered cycloalkyl or heterocyclyl ring;

R¹ and R² are, at each occurence, optionally, together with the atom to which they are connected, form an optionally substituted 3-20 membered cycloalkyl or heterocyclyl ring;
R³ is selected from OR¹³, SR¹³, NR¹³R¹⁴, CH₂NR¹³R¹⁴, CH₂CH₂NR¹³R¹⁴, C₁-C₈ alkylene NR¹³R¹⁴, C(O)R¹³, C(O)OR¹³, C(O)NR¹³R¹⁴, S(O)R¹³, S(O)₂R¹⁴, S(O)₂NR¹³R¹⁴, NR¹⁵C(O)OR¹³, NR¹⁵C(O)R¹³, NR¹⁵C(O)NR¹³R¹⁴, NR¹⁵S(O)R³², NR¹⁵S(O)₂R³², NR¹⁵S(O)₂NR¹³R¹⁴ optionally substituted C₁-C₈ alkylene, optionally substituted C₂-C₈ alkenylene, optionally substituted C₂-C₈ alkynylene, optionally substituted C₁-C₈alkoxyC₁-C₈alkylene, optionally substituted C₁-C₈alkylaminoC₁-C₈alkylene, optionally substituted 3-8 membered cycloalkylene, optionally substituted 3-8 membered heterocyclylene, optionally substituted aryl, and optionally substituted heteroaryl, wherein
R¹³ is null, or a bivalent moiety selected from optionally substituted C₁-C₈ alkylenyl, optionally substituted C₂-C₈ alkenylene, optionally substituted C₂-C₈ alkynylene, optionally substituted C₃-C₈ cycloalkylene, optionally substituted 3-8 membered heterocyclylene, optionally substituted arylene, and optionally substituted heteroarylene;
R¹⁴ and R¹⁵ are independently selected from optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₈ cycloalkyl, optionally substituted 3-8 membered heterocyclyl, optionally substituted C₂-C₈ alkenyl, optionally substituted C₂-C₈ alkynyl, optionally substituted aryl, and optionally substituted heteroaryl; or
R¹³ and R¹⁴, R¹³ and R¹⁵, R¹⁴ and R¹⁵ together with the atom to which they are connected form a 3-20 membered cycloalkyl or heterocyclyl ring;
   R⁴ is, at each occurence, independently selected from hydrogen, halogen, oxo, CN, NO₂, OR¹⁶, SR¹⁶, NR¹⁶R¹⁷, C(O)R¹⁶, C(O)OR¹⁶, C(O)NR¹⁶R¹⁷, S(O)R¹⁶, S(O)₂R¹⁶, S(O)₂NR¹⁶R¹⁷, NR¹⁸C(O)OR¹⁶, NR¹⁸C(O)R¹⁶, NR¹⁸C(O)NR¹⁶R¹⁷, NR¹⁸S(O)R¹⁶, NR¹⁸S(O)₂R¹⁶, NR¹⁸S(O)₂NR¹⁶R¹⁵, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl; wherein

R¹⁶, R¹⁷, and R¹⁸ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl; or

R¹⁶ and R¹⁷, R¹⁶ and R¹⁸, R¹⁷ and R¹⁸ together with the atom to which they are connected form an optionally substituted 3-20 membered cycloalkyl or heterocyclyl ring; and pharmaceutically acceptable salts thereof.

When the “Linker” moiety of the bivalent compound is attached to R¹;
R¹ is selected from OR¹⁹, SR¹⁹, NR¹⁹R²⁰, CH₂NR¹⁹R²⁰, CH₂CH₂NR¹⁹R²⁰, C₁-C₈ alkylene NR¹⁹R²⁰, C(O)R¹⁹, C(O)OR¹⁹, C(O)NR¹⁹R²⁰, S(O)R¹⁹, S(O)₂R²⁰, S(O)₂NR¹⁹R²⁰, NR²¹C(O)OR¹⁹, NR²¹C(O)R¹⁹, NR²¹C(O)NR¹⁹R²⁰, NR²¹S(O)R¹⁹, NR²¹S(O)₂R¹⁹, NR²¹S(O)₂NR¹⁹R²⁰ optionally substituted C₁-C₈ alkylene, optionally substituted C₂-C₈ alkenylene, optionally substituted C₂-C₈ alkynylene, optionally substituted C₁-C₈alkoxyC₁-C₈alkylene, optionally substituted C₁-C₈alkylaminoC₁-C₈alkylene, optionally substituted 3-8 membered cycloalkylene, optionally substituted 3-8 membered heterocyclylene, optionally substituted aryl, and optionally substituted heteroaryl, wherein
R¹⁹ is null, or a bivalent moiety selected from optionally substituted C₁-C₈ alkylenyl, optionally substituted C₂-C₈ alkenylene, optionally substituted C₂-C₈ alkynylene, optionally substituted C₃-C₈ cycloalkylene, optionally substituted 3-8 membered heterocyclylene, optionally substituted arylene, and optionally substituted heteroarylene;
R²⁰ and R²¹ are independently selected from optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₈ cycloalkyl, optionally substituted 3-8 membered heterocyclyl, optionally substituted C₂-C₈ alkenyl, optionally substituted C₂-C₈ alkynyl, optionally substituted aryl, and optionally substituted heteroaryl; or
R¹⁹ and R²⁰, R¹⁹ and R²¹, R²⁰ and R²¹ together with the atom to which they are connected form a 3-20 membered cycloalkyl or heterocyclyl ring;
   R² is, at each occurence, independently selected from null, hydrogen, C(O)R¹⁰, C(O)OR¹⁰, C(O)NR¹⁰R¹¹, S(O)R¹⁰, S(O)₂R¹⁰, S(O)₂NR¹⁰R¹¹, NR¹²C(O)OR¹⁰, NR¹²C(O)R¹⁰, NR¹²C(O)NR¹⁰R¹¹, NR¹²S(O)R¹⁰, NR¹²S(O)₂R¹°, NR¹²S(O)₂NR¹⁰R¹¹, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl; wherein
   R¹⁰, R¹¹, and R¹² are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl; or

R¹⁰ and R¹¹, R¹⁰ and R¹², R¹¹ and R¹² together with the atom to which they are connected form an optionally substituted 3-20 membered cycloalkyl or heterocyclyl ring;

R³ and R⁴ are, at each occurence, independently selected from hydrogen, halogen, oxo, CN, NO₂, OR¹⁶, SR¹⁶, NR¹⁶R¹⁷, C(O)R¹⁶, C(O)OR¹⁶, C(O)NR¹⁶R¹⁷, S(O)R¹⁶, S(O)₂R¹⁶, S(O)₂NR¹⁶R¹⁷, NR¹⁸C(O)OR¹⁶, NR¹⁸C(O)R¹⁶, NR¹⁸C(O)NR¹⁶R¹⁷, NR¹⁸S(O)R¹⁶, NR¹⁸S(O)₂R¹⁶, NR¹⁸S(O)₂NR¹⁶R⁵, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl; wherein

R¹⁶, R¹⁷, and R¹⁸ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl; or

R¹⁶ and R¹⁷, R¹⁶ and R¹⁸, R¹⁷ and R¹⁸ together with the atom to which they are connected form an optionally substituted 3-20 membered cycloalkyl or heterocyclyl ring; and pharmaceutically acceptable salts thereof.

In another embodiment, (HPK1) ligands include a moiety according to FORMULA 3′:

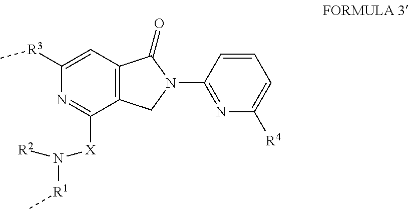

wherein X is absent or when present is CH₂,
the “Linker” moiety of the bivalent compound is attached independently to R¹ or R³ (indicated by dotted line);
when the “Linker” moiety of the bivalent compound is attached to R³;
R¹ and R² are, at each occurrence, independently selected from null, hydrogen, C(O)R¹⁰, C(O)OR¹⁰, C(O)NR¹⁰R¹¹, S(O)R¹⁰, S(O)₂R¹⁰, S(O)₂NR¹⁰R¹¹, NR¹²C(O)OR¹⁰, NR¹²C(O)R¹⁰, NR¹²C(O)NR¹⁰R¹¹, NR¹²S(O)R¹⁰, NR¹²S(O)₂R¹⁰, NR¹²S(O)₂NR¹⁰R¹¹, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl; wherein
R¹⁰, R¹¹, and R¹² are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl; or

R¹⁰ and R¹¹, R¹⁰ and R¹², R¹¹ and R¹² together with the atom to which they are connected form an optionally substituted 3-20 membered cycloalkyl or heterocyclyl ring; R¹ and R² are, at each occurrence, optionally, together with the atom to which they are connected, form an optionally substituted 3-20 membered cycloalkyl or heterocyclyl ring; R³ is selected from OR¹³, SR¹³, NR¹³R¹⁴, CH₂NR¹³R¹⁴, CH₂CH₂NR¹³R¹⁴, C₁-C₈ alkylene NR¹³R¹⁴, C(O)R¹³, C(O)OR¹³, C(O)NR¹³R¹⁴, S(O)R¹³, S(O)₂R¹⁴, S(O)₂NR¹³R¹⁴, NR¹⁵C(O)OR¹³, NR¹⁵C(O)R¹³, NR¹⁵C(O)NR¹³R¹⁴, NR¹⁵S(O)R³², NR¹⁵S(O)₂R³², NR¹⁵S(O)₂NR¹³R¹⁴ optionally substituted C₁-C₈ alkylene, optionally substituted C₂-C₈ alkenylene, optionally substituted C₂-C₈ alkynylene, optionally substituted C₁-C₈alkoxyC₁-C₈alkylene, optionally substituted C₁-C₈alkylaminoC₁-C₈alkylene, optionally substituted 3-8 membered cycloalkylene, optionally substituted 3-8 membered heterocyclylene, optionally substituted aryl, and optionally substituted heteroaryl, wherein
R¹³ is null, or a bivalent moiety selected from optionally substituted C₁-C₈ alkylenyl, optionally substituted C₂-C₈ alkenylene, optionally substituted C₂-C₈ alkynylene, optionally substituted C₃-C₈ cycloalkylene, optionally substituted 3-8 membered heterocyclylene, optionally substituted arylene, and optionally substituted heteroarylene;
R¹⁴ and R¹⁵ are independently selected from optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₈ cycloalkyl, optionally substituted 3-8 membered heterocyclyl, optionally substituted C₂-C₈ alkenyl, optionally substituted C₂-C₈ alkynyl, optionally substituted aryl, and optionally substituted heteroaryl; or
R¹³ and R¹⁴, R¹³ and R¹⁵, R¹⁴ and R¹⁵ together with the atom to which they are connected form a 3-20 membered cycloalkyl or heterocyclyl ring;
   R⁴ is at each occurrence, independently selected from hydrogen, halogen, oxo, CN, NO₂, OR¹⁶, SR¹⁶, NR¹⁶R¹⁷, C(O)R¹⁶, C(O)OR¹⁶, C(O)NR¹⁶R¹⁷, S(O)R¹⁶, S(O)₂R¹⁶, S(O)₂NR¹⁶R¹⁷, NR¹⁸C(O)OR¹⁶, NR¹⁸C(O)R¹⁶, NR¹⁸C(O)NR¹⁶R¹⁷, NR¹⁸S(O)R¹⁶, NR¹⁸S(O)₂R¹⁶, NR¹⁸S(O)₂NR¹⁶R¹⁵, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl; wherein

R¹⁶, R¹⁷, and R¹⁸ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl; or

R¹⁶ and R¹⁷, R¹⁶ and R¹⁸, R¹⁷ and R¹⁸ together with the atom to which they are connected form an optionally substituted 3-20 membered cycloalkyl or heterocyclyl ring;

when the “Linker” moiety of the bivalent compound is attached to R¹;
R¹ is selected from OR¹⁹, SR¹⁹, NR¹⁹R²⁰, CH₂NR¹⁹R²⁰, CH₂CH₂NR¹⁹R²⁰, C₁-C₈ alkylene NR¹⁹R²⁰, C(O)R¹⁹, C(O)OR¹⁹, C(O)NR¹⁹R²⁰, S(O)R¹⁹, S(O)₂R²⁰, S(O)₂NR¹⁹R²⁰, NR²¹C(O)OR¹⁹, NR²¹C(O)R¹⁹, NR²¹C(O)NR¹⁹R²⁰, NR²¹S(O)R¹⁹, NR²¹S(O)₂R¹⁹, NR²¹S(O)₂NR¹⁹R²⁰ optionally substituted C₁-C₈ alkylene, optionally substituted C₂-C₈ alkenylene, optionally substituted C₂-C₈ alkynylene, optionally substituted C₁-C₈alkoxyC₁-C₈alkylene, optionally substituted C₁-C₈alkylaminoC₁-C₈alkylene, optionally substituted 3-8 membered cycloalkylene, optionally substituted 3-8 membered heterocyclylene, optionally substituted aryl, and optionally substituted heteroaryl, wherein
R¹⁹ is null, or a bivalent moiety selected from optionally substituted C₁-C₈ alkylenyl, optionally substituted C₂-C₈ alkenylene, optionally substituted C₂-C₈ alkynylene, optionally substituted C₃-C₈ cycloalkylene, optionally substituted 3-8 membered heterocyclylene, optionally substituted arylene, and optionally substituted heteroarylene;
R²⁰ and R²¹ are independently selected from optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₈ cycloalkyl, optionally substituted 3-8 membered heterocyclyl, optionally substituted C₂-C₈ alkenyl, optionally substituted C₂-C₈ alkynyl, optionally substituted aryl, and optionally substituted heteroaryl; or
R¹⁹ and R²⁰, R¹⁹ and R²¹, R²⁰ and R²¹ together with the atom to which they are connected form a 3-20 membered cycloalkyl or heterocyclyl ring;
   R² is, at each occurrence, independently selected from null, hydrogen, C(O)R¹⁰, C(O)OR¹⁰, C(O)NR¹⁰R¹¹, S(O)R¹⁰, S(O)₂R¹⁰, S(O)₂NR¹⁰R¹¹, NR¹²C(O)OR¹⁰, NR¹²C(O)R¹⁰, NR¹²C(O)NR¹⁰R¹¹, NR¹²S(O)R¹⁰, NR¹²S(O)₂R¹°, NR¹²S(O)₂NR¹⁰R¹¹, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl; wherein
   R¹⁰, R¹¹, and R¹² are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl; or

R¹⁰ and R¹¹, R¹⁰ and R¹², R¹¹ and R¹² together with the atom to which they are connected form an optionally substituted 3-20 membered cycloalkyl or heterocyclyl ring;

R³ and R⁴ are, at each occurrence, independently selected from hydrogen, halogen, oxo, CN, NO₂, OR¹⁶, SR¹⁶, NR¹⁶R¹⁷, C(O)R¹⁶, C(O)OR¹⁶, C(O)NR¹⁶R¹⁷, S(O)R¹⁶, S(O)₂R¹⁶, S(O)₂NR¹⁶R¹⁷, NR¹⁸C(O)OR¹⁶, NR¹⁸C(O)R¹⁶, NR¹⁸C(O)NR¹⁶R¹⁷, NR¹⁸S(O)R¹⁶, NR¹⁸S(O)₂R¹⁶, NR¹⁸S(O)₂NR¹⁶R¹⁵, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl; wherein

R¹⁶, R¹⁷, and R¹⁸ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl; or

R¹⁶ and R¹⁷, R¹⁶ and R¹⁸, R¹⁷ and R¹⁸ together with the atom to which they are connected form an optionally substituted 3-20 membered cycloalkyl or heterocyclyl ring;

In another embodiment, (HPK1) ligands include a moiety according to FORMULA 3A:

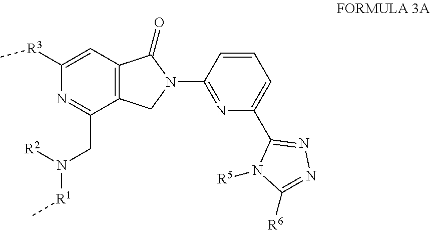

wherein
the “Linker” moiety of the bivalent compound is attached independently to R¹ or R³ (indicated by dotted line);
The definitions of, R¹, R² and R³ are the same as for FORMULA 3;
R⁵ is, at each occurence, independently selected from null, hydrogen, C(O)R²², C(O)OR²²C(O)NR²²R²³, S(O)R²², S(O)₂R²², S(O)₂NR²²R²³, NR²⁴C(O)OR²², NR²⁴C(O)R²², NR²⁴C(O)NR²²R²³, NR²⁴S(O)R²², NR²⁴S(O)₂R²², NR²⁴S(O)₂NR²²R²³, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl; wherein

R²², R²³, and R²⁴ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl; or

R²² and R²³, R²² and R²⁴, R²³ and R²⁴ together with the atom to which they are connected form an optionally substituted 3-20 membered cycloalkyl or heterocyclyl ring;

R⁶ is, at each occurence, independently selected from hydrogen, halogen, oxo, CN, NO₂, OR²⁵, SR²⁵, NR²⁵R²⁶, C(O)R²⁵, C(O)OR²⁵, C(O)NR²⁵R²⁶, S(O)R²⁵, S(O)₂R²⁵, S(O)₂NR²⁵R²⁶, NR²⁷C(O)OR²⁵, NR²⁷C(O)R²⁵, NR²¹C(O)NR²⁵R²⁶, NR²⁷S(O)R²⁵, NR²⁷S(O)₂R²⁵, NR²⁷S(O)₂NR²⁵R²⁶, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl; wherein

R²⁵, R²⁶, and R²⁷ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl; or

R²⁵ and R²⁶, R²⁵ and R²⁶, R²⁶ and R²⁷ together with the atom to which they are connected form an optionally substituted 3-20 membered cycloalkyl or heterocyclyl ring; and pharmaceutically acceptable salts thereof.

In another embodiment, (HPK1) ligands include a moiety according to FORMULA 3A′:

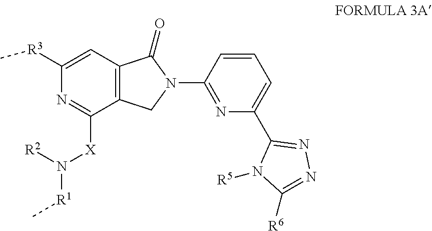

wherein X is absent or when present is CH₂,
the “Linker” moiety of the bivalent compound is attached independently to R¹ or R³ (indicated by dotted line);
the definitions of, R¹, R² and R³ are the same as for FORMULA 3;
R⁵ is, at each occurrence, independently selected from null, hydrogen, C(O)R²², C(O)OR²², C(O)NR²²R²³, S(O)R²², S(O)₂R²², S(O)₂NR²²R²³, NR²⁴C(O)OR²², N²⁴C(O)R²², NR²⁴C(O)NR²²R²³, NR²⁴S(O)R²², NR²⁴S(O)₂R²², NR²⁴S(O)₂NR²²R²³, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl; wherein

R²², R²³, and R²⁴ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl; or

R²² and R²³, R²² and R²⁴, R²³ and R²⁴ together with the atom to which they are connected form an optionally substituted 3-20 membered cycloalkyl or heterocyclyl ring;

R⁶ is, at each occurrence, independently selected from hydrogen, halogen, oxo, CN, NO₂, OR²⁵, SR²⁵, NR²⁵R²⁶, C(O)R²⁵, C(O)OR²⁵, C(O)NR²⁵R²⁶, S(O)R²⁵, S(O)₂R²⁵, S(O)₂NR²⁵R²⁶, NR²⁷C(O)OR²⁵, NR²⁷C(O)R²⁵, NR²¹C(O)NR²⁵R²⁶, NR²⁷S(O)R²⁵, NR²⁷S(O)₂R²⁵, NR²⁷S(O)₂NR²⁵R²⁶, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl; wherein

R²⁵, R²⁶, and R²⁷ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl; or

R²⁵ and R²⁶, R²⁵ and R²⁶, R²⁶ and R²⁷ together with the atom to which they are connected form an optionally substituted 3-20 membered cycloalkyl or heterocyclyl ring;

In another embodiment, (HPK1) ligands include a moiety according to FORMULA 3B:

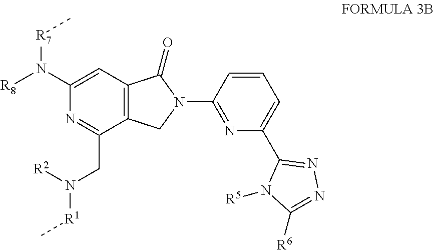

wherein
the “Linker” moiety of the bivalent compound is attached independently to R¹ or R⁷ (indicated by dotted line);
The definitions of R⁵ and R⁶ are the same as for FORMULA 3A;
When the “Linker” moiety of the bivalent compound is attached to R¹;
R¹ is selected from OR²⁸, SR²⁸, NR²⁸R²⁹, CH₂NR²⁸R²⁹, CH₂CH₂NR²⁸R²⁹, C₁-C₈ alkylene NR²⁸R²⁹, C(O)R²⁸, C(O)OR²⁸, C(O)NR²⁸R²⁹, S(O)R²⁸, S(O)₂R²⁹, S(O)₂NR²⁸R²⁹, NR³⁰C(O)OR²⁸, NR³⁰C(O)R²⁸, NR³⁰C(O)NR²⁸R²⁹, NR³⁰S(O)R²⁸, NR³⁰S(O)₂R²⁸, NR³⁰S(O)₂NR²⁸R²⁹ optionally substituted C₁-C₈ alkylene, optionally substituted C₂-C₈ alkenylene, optionally substituted C₂-C₈ alkynylene, optionally substituted C₁-C₈alkoxyC₁-C₈alkylene, optionally substituted C₁-C₈alkylaminoC₁-C₈alkylene, optionally substituted 3-8 membered cycloalkylene, optionally substituted 3-8 membered heterocyclylene, optionally substituted aryl, and optionally substituted heteroaryl, wherein
R²⁸ is null, or a bivalent moiety selected from optionally substituted C₁-C₈ alkylenyl, optionally substituted C₂-C₈ alkenylene, optionally substituted C₂-C₈ alkynylene, optionally substituted C₃-C₈ cycloalkylene, optionally substituted 3-8 membered heterocyclylene, optionally substituted arylene, and optionally substituted heteroarylene;
R²⁹ and R³⁰ are independently selected from optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₈ cycloalkyl, optionally substituted 3-8 membered heterocyclyl, optionally substituted C₂-C₈ alkenyl, optionally substituted C₂-C₈ alkynyl, optionally substituted aryl, and optionally substituted heteroaryl; or
R²⁸ and R²⁹, R²⁸ and R²⁹, R²⁹ and R³⁰ together with the atom to which they are connected form a 3-20 membered cycloalkyl or heterocyclyl ring;
   R², R⁷ and R⁸ are, at each occurence, independently selected from null, hydrogen, C(O)R³¹, C(O)OR³¹, C(O)NR³¹R³², S(O)R³¹, S(O)₂R³¹, S(O)₂NR³¹R³², NR³³C(O)OR³¹, NR³³C(O)R³¹, NR³³C(O)NR³¹R³², NR³³S(O)R³¹, NR³³S(O)₂R³¹, NR³³S(O)₂NR³¹R³², optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl; wherein

R³¹, R³², and R³³ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl; or

R³¹ and R³², R³¹ and R³³, R³² and R³³ together with the atom to which they are connected form an optionally substituted 3-20 membered cycloalkyl or heterocyclyl ring;

R⁷ and R⁸ are, at each occurence, optionally, together with the atom to which they are connected, form an optionally substituted 3-20 membered cycloalkyl or heterocyclyl ring;
and pharmaceutically acceptable salts thereof.
When the “Linker” moiety of the bivalent compound is attached to R⁷; R⁷ is selected from OR²⁸, SR²⁸, NR²⁸R²⁹, CH₂NR²⁸R²⁹, CH₂CH₂NR²⁸R²⁹, C₁-C₈ alkylene NR²⁸R²⁹, C(O)R²⁸, C(O)OR²⁸, C(O)NR²⁸R²⁹, S(O)R²⁸, S(O)₂R²⁹, S(O)₂NR²⁸R²⁹, NR³⁰C(O)OR²⁸, NR³⁰C(O)R²⁸, NR³⁰C(O)NR²⁸R²⁹, NR³⁰S(O)R²⁸, NR³⁰S(O)₂R²⁸, NR³⁰S(O)₂NR²⁸R²⁹ optionally substituted C₁-C₈ alkylene, optionally substituted C₂-C₈ alkenylene, optionally substituted C₂-C₈ alkynylene, optionally substituted C₁-C₈alkoxyC₁-C₈alkylene, optionally substituted C₁-C₈alkylaminoC₁-C₈alkylene, optionally substituted 3-8 membered cycloalkylene, optionally substituted 3-8 membered heterocyclylene, optionally substituted aryl, and optionally substituted heteroaryl, wherein
R²⁸ is null, or a bivalent moiety selected from optionally substituted C₁-C₈ alkylenyl, optionally substituted C₂-C₈ alkenylene, optionally substituted C₂-C₈ alkynylene, optionally substituted C₃-C₈ cycloalkylene, optionally substituted 3-8 membered heterocyclylene, optionally substituted arylene, and optionally substituted heteroarylene;
R²⁹ and R³⁰ are independently selected from optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₈ cycloalkyl, optionally substituted 3-8 membered heterocyclyl, optionally substituted C₂-C₈ alkenyl, optionally substituted C₂-C₈ alkynyl, optionally substituted aryl, and optionally substituted heteroaryl; or
R²⁸ and R²⁹, R²⁸ and R²⁹, R²⁹ and R³⁰ together with the atom to which they are connected form a 3-20 membered cycloalkyl or heterocyclyl ring;
   R¹, R² and R⁸ are, at each occurence, independently selected from null, hydrogen, C(O)R³¹, C(O)OR³¹, C(O)NR³¹R³², S(O)R³¹, S(O)₂R³¹, S(O)₂NR³¹R³², NR³³C(O)OR³¹, NR³³C(O)R³¹, NR³³C(O)NR³¹R³², NR³³S(O)R³¹, NR³³S(O)₂R³¹, NR³³S(O)₂NR³¹R³², optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl; wherein

R³¹, R³², and R³³ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl; or

R³¹ and R³², R³¹ and R³³, R³² and R³³ together with the atom to which they are connected form an optionally substituted 3-20 membered cycloalkyl or heterocyclyl ring;

R¹ and R² are, at each occurence, optionally, together with the atom to which they are connected, form an optionally substituted 3-20 membered cycloalkyl or heterocyclyl ring; and pharmaceutically acceptable salts thereof.
In another embodiment, (HPK1) ligands include a moiety according to FORMULA 4:

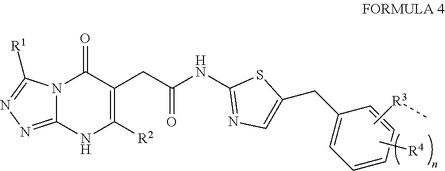

wherein
the “Linker” moiety of the bivalent compound is attached independently to R³ (indicated by dotted line);
R^{1′} R² and R⁴ are, at each occurence, independently selected from hydrogen, halogen, oxo, CN, NO₂, OR⁵, SR⁵, NR⁵R⁶, C(O)R⁵, C(O)OR⁵, C(O)NR⁵R⁶, S(O)R⁵, S(O)₂R⁵, S(O)₂NR⁵R⁶, NR⁷C(O)OR⁵, NR⁷C(O)R⁵, NR⁷C(O)NR²⁵R²⁶, NR⁷S(O)R⁵, NR⁷S(O)₂R⁵, NR⁷S(O)₂NR⁵R⁶ optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl; wherein

R⁵, R⁶, and R⁷ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl; or

R⁵ and R⁶, R⁵ and R⁷, R⁶ and R⁷ together with the atom to which they are connected form an optionally substituted 3-20 membered cycloalkyl or heterocyclyl ring;

n is independently selected from 0, 1, 2, 3 and 4;
R³ is selected from OR⁸, SR⁸, NR⁸R⁹, CH₂NR⁸R⁹, CH₂CH₂NR⁸R⁹, C₁-C₈ alkylene NR⁸R⁹, C(O)R⁸, C(O)OR⁹, C(O)NR⁸R⁹, S(O)R⁸, S(O)₂R⁹, S(O)₂NR⁸R⁹, NR¹⁰C(O)OR⁹, NR¹⁰C(O)R⁹, NR¹⁰C(O)NR⁸R⁹, NR¹⁰S(O)R⁸, NR¹⁰S(O)₂R⁸, NR¹⁰S(O)₂NR⁸R⁹ optionally substituted C₁-C₈ alkylene, optionally substituted C₂-C₈ alkenylene, optionally substituted C₂-C₈ alkynylene, optionally substituted C₁-C₈alkoxyC₁-C₈alkylene, optionally substituted C₁-C₈alkylaminoC₁-C₈alkylene, optionally substituted 3-8 membered cycloalkylene, optionally substituted 3-8 membered heterocyclylene, optionally substituted aryl, and optionally substituted heteroaryl, wherein
R⁸ is null, or a bivalent moiety selected from optionally substituted C₁-C₈ alkylenyl, optionally substituted C₂-C₈ alkenylene, optionally substituted C₂-C₈ alkynylene, optionally substituted C₃-C₈ cycloalkylene, optionally substituted 3-8 membered heterocyclylene, optionally substituted arylene, and optionally substituted heteroarylene;
R⁹ and R¹⁰ are independently selected from optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₈ cycloalkyl, optionally substituted 3-8 membered heterocyclyl, optionally substituted C₂-C₈ alkenyl, optionally substituted C₂-C₈ alkynyl, optionally substituted aryl, and optionally substituted heteroaryl; or
R⁸ and R⁹, R⁸ and R¹⁰, R⁹ and R¹⁰ together with the atom to which they are connected form a 3-20 membered cycloalkyl or heterocyclyl ring;
   and pharmaceutically acceptable salts thereof.
   In another embodiment, (HPK1) ligands include a moiety according to FORMULA 4A:

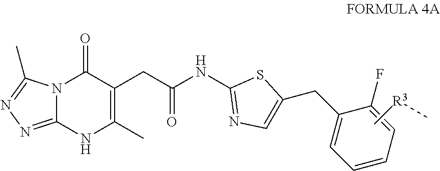

wherein
the “Linker” moiety of the bivalent compound is attached independently to R³ (indicated by dotted line);
The definitions of R³ is the same as for FORMULA 4.
In an embodiment, (HIPK1) ligands are selected from the group consisting of:

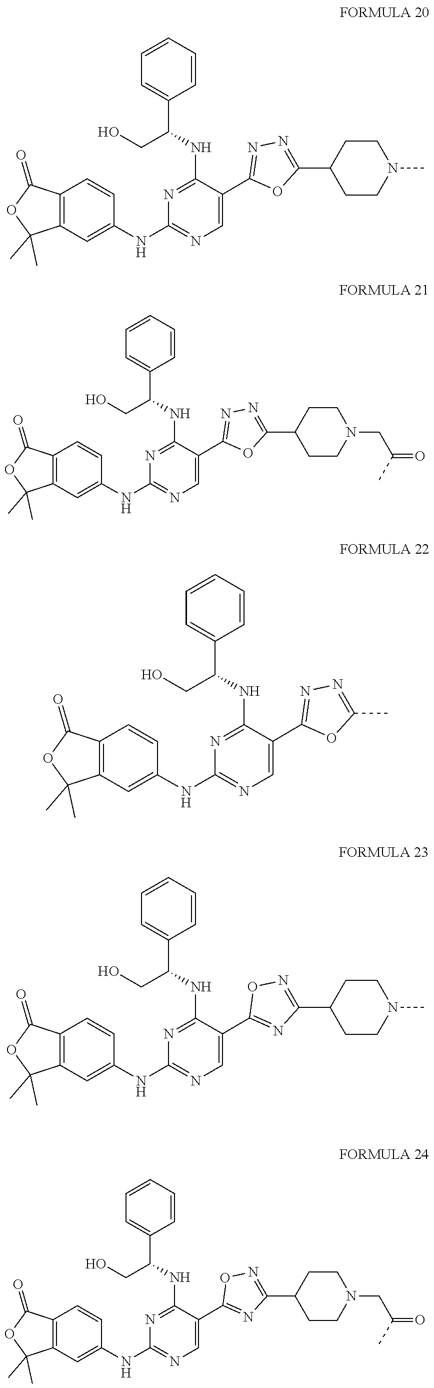

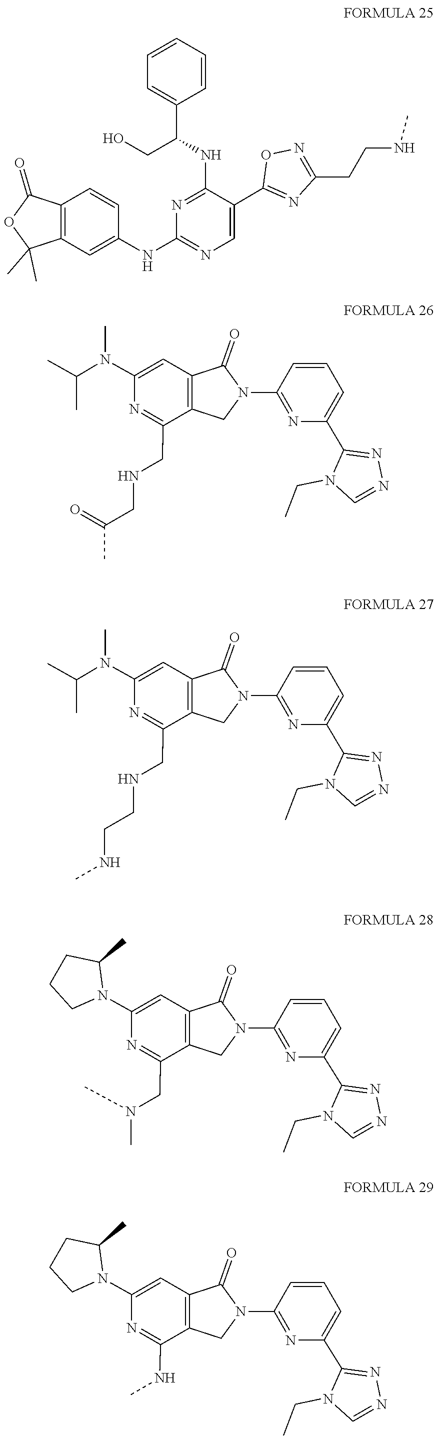

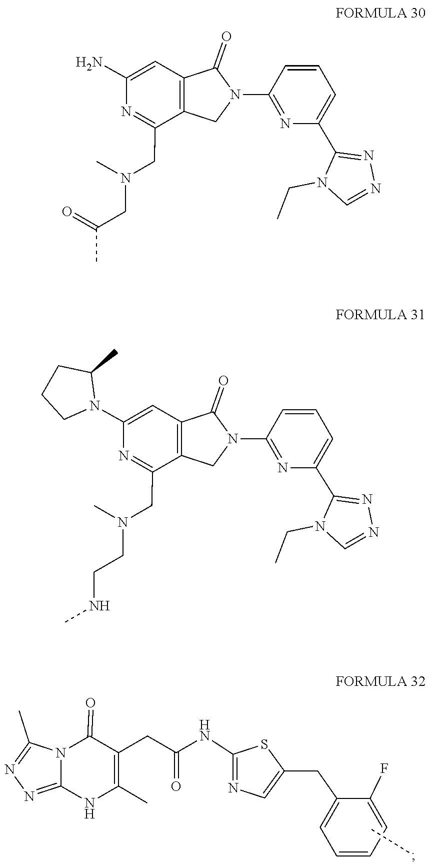

and
pharmaceutically acceptable salts thereof

### Degradation/Disruption Tags

Degradation/Disruption tags (EL) include, but are not limited to:
In an embodiment, degradation/disruption tags include a moiety according to FORMULAE 12A, 12B, 12C and 12D:

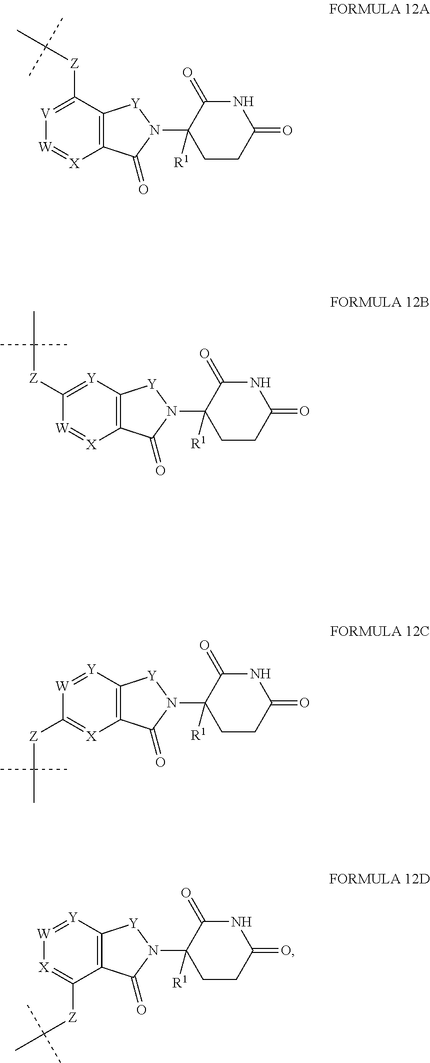

wherein
V, W, and X are independently selected from CR² and N;
Y is selected from CO, CR³R⁴, and N═N;
Z is selected from null, CO, CR⁵R⁶, NR⁵, O, optionally substituted C₁-C₁₀ alkylene, optionally substituted C₁-C₁₀ alkenylene, optionally substituted C₁-C₁₀ alkynylene, optionally substituted 3-membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl; preferably, Z is selected from null, CH₂, CH═CH, C≡C, NH and O;
R¹, and R² are independently selected from hydrogen, halogen, cyano, nitro, optionally substituted C₁-C₆ alkyl, optionally substituted 3 to 6 membered carbocyclyl, and optionally substituted 4 to 6 membered heterocyclyl;
R³, and R⁴ are independently selected from hydrogen, halogen, cyano, nitro, optionally substituted C₁-C₆ alkyl, optionally substituted 3 to 6 membered carbocyclyl, and optionally substituted 4 to 6 membered heterocyclyl; or R³ and R⁴ together with the atom to which they are connected form a 3-6 membered carbocyclyl, or 4-6 membered heterocyclyl; and
R⁵ and R⁶ are independently selected from null, hydrogen, halogen, oxo, hydroxyl, amino, cyano, nitro, optionally substituted C₁-C₆ alkyl, optionally substituted 3 to 6 membered carbocyclyl, and optionally substituted 4 to 6 membered heterocyclyl; or R⁵ and R⁶ together with the atom to which they are connected form a 3-6 membered carbocyclyl, or 4-6 membered heterocyclyl.
In an embodiment, degradation/disruption tags include a moiety according to one of FORMULAE 12E, 12F, 12G, 12H, and 12I:

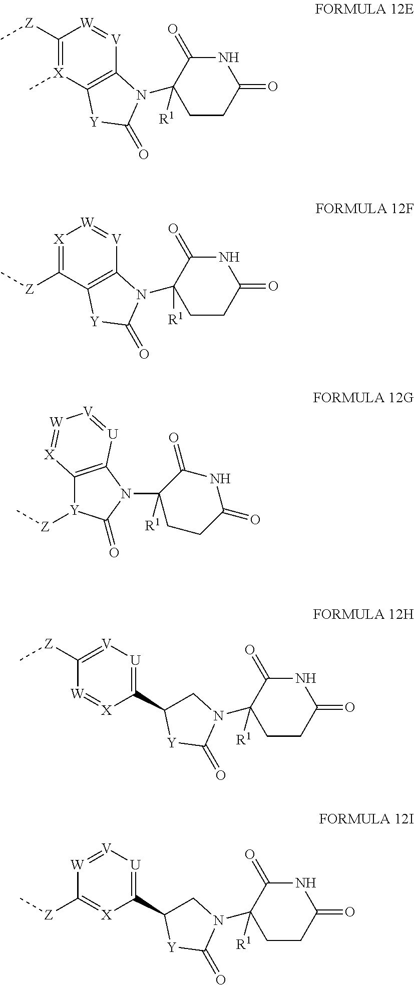

wherein
U, V, W, and X are independently selected from CR² and N;
Y is selected from CR³R⁴, NR³ and O; preferably, Y is selected from CH₂, NH, NCH₃ and O;
Z is selected from null, CO, CR⁵R⁶, NR⁵, O, optionally substituted C₁-C₁₀ alkylene, optionally substituted C₁-C₁₀ alkenylene, optionally substituted C₁-C₁₀ alkynylene, optionally substituted 3-membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl; preferably, Z is selected from null, CH₂, CH═CH, C≡C, NH and O;
R¹, and R² are independently selected from hydrogen, halogen, cyano, nitro, optionally substituted C₁-C₆ alkyl, optionally substituted 3 to 6 membered carbocyclyl, and optionally substituted 4 to 6 membered heterocyclyl;
R³, and R⁴ are independently selected from hydrogen, halogen, cyano, nitro, optionally substituted C₁-C₆ alkyl, optionally substituted 3 to 6 membered carbocyclyl, and optionally substituted 4 to 6 membered heterocyclyl; or R³ and R⁴ together with the atom to which they are connected form a 3-6 membered carbocyclyl, or 4-6 membered heterocyclyl; and
R⁵ and R⁶ are independently selected from null, hydrogen, halogen, oxo, hydroxyl, amino, cyano, nitro, optionally substituted C₁-C₆ alkyl, optionally substituted 3 to 6 membered carbocyclyl, and optionally substituted 4 to 6 membered heterocyclyl; or R⁵ and R⁶ together with the atom to which they are connected form a 3-6 membered carbocyclyl, or 4-6 membered heterocyclyl; and

pharmaceutically acceptable salts thereof.

In an embodiment, degradation/disruption tags include a moiety according to FORMULA 13A:

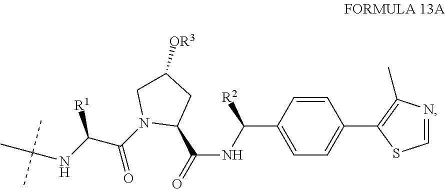

wherein
R¹ and R² are independently selected from hydrogen, optionally substituted C₁-C₈ alkyl, optionally substituted C₁-C₈alkoxyC₁-C₈alkyl, optionally substituted C₁-C₈ haloalkyl, optionally substituted C₁-C₈ hydroxyalkyl, optionally substituted C₁-C₈ aminoalkyl, optionally substituted C₁-C₈alkylaminoC₁-C₈alkyl, optionally substituted C₃-C₇ cycloalkyl, optionally substituted 3-7 membered heterocyclyl, optionally substituted C₂-C₈ alkenyl, and optionally substituted C₂-C₈ alkynyl; and
R³ is hydrogen, optionally substituted C(O)C₁-C₈ alkyl, optionally substituted C(O)C₁-C₈alkoxyC₁-C₈alkyl, optionally substituted C(O)C₁-C₈ haloalkyl, optionally substituted C(O)C₁-C₈ hydroxyalkyl, optionally substituted C(O)C₁-C₈ aminoalkyl, optionally substituted C(O)C₁-C₈alkylaminoC₁-C₈alkyl, optionally substituted C(O)C₃-C₇ cycloalkyl, optionally substituted C(O)(3-7 membered heterocyclyl), optionally substituted C(O)C₂-C₈ alkenyl, optionally substituted C(O)C₂-C₈ alkynyl, optionally substituted C(O)OC₁-C₈alkoxyC₁-C₈alkyl, optionally substituted C(O)OC₁-C₈ haloalkyl, optionally substituted C(O)OC₁-C₈ hydroxyalkyl, optionally substituted C(O)OC₁-C₈ aminoalkyl, optionally substituted C(O)OC₁-C₈alkylaminoC₁-C₈alkyl, optionally substituted C(O)OC₃-C₇ cycloalkyl, optionally substituted C(O)O(3-7 membered heterocyclyl), optionally substituted C(O)OC₂-C₈ alkenyl, optionally substituted C(O)OC₂-C₈ alkynyl, optionally substituted C(O)NC₁-C₈alkoxyC₁-C₈alkyl, optionally substituted C(O)NC₁-C₈ haloalkyl, optionally substituted C(O)NC₁-C₈ hydroxyalkyl, optionally substituted C(O)NC₁-C₈ aminoalkyl, optionally substituted C(O)NC₁-C₈alkylaminoC₁-C₈alkyl, optionally substituted C(O)NC₃-C₇ cycloalkyl, optionally substituted C(O)N(3-7 membered heterocyclyl), optionally substituted C(O)NC₂-C₈ alkenyl, optionally substituted C(O)NC₂-C₈ alkynyl, optionally substituted P(O)(OH)₂, optionally substituted P(O)(OC₁-C₈ alkyl)₂, and optionally substituted P(O)(OC₁-C₈ aryl)₂.
In an embodiment, degradation/disruption tags include a moiety according to FORMULAE 13B, 13C, 13D, 13E and 13F:

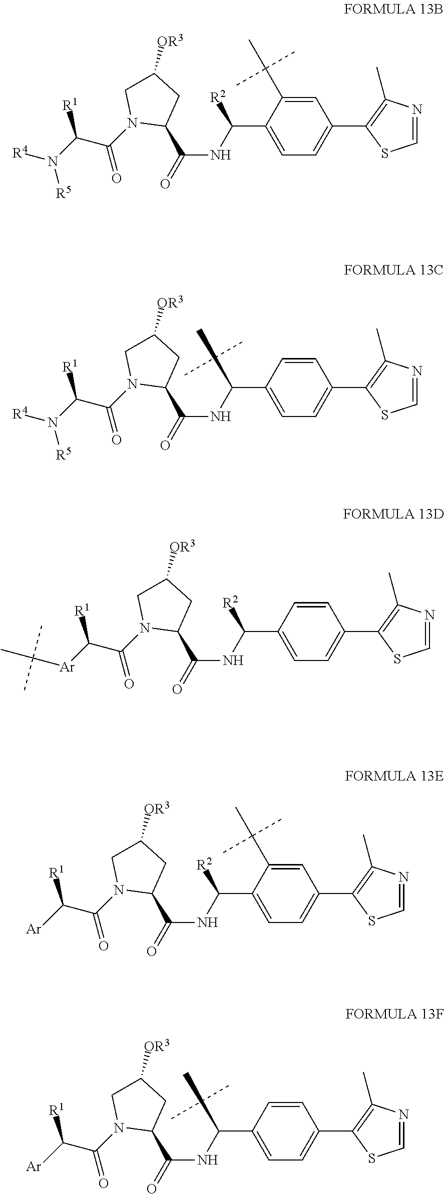

wherein
R¹ and R² are independently selected from hydrogen, halogen, OH, NH₂, CN, optionally substituted C₁-C₈ alkyl, optionally substituted C₁-C₈alkoxyC₁-C₈alkyl, optionally substituted C₁-C₈ haloalkyl, optionally substituted C₁-C₈ hydroxyalkyl, optionally substituted C₁-C₈ aminoalkyl, optionally substituted C₁-C₈alkylaminoC₁-C₈alkyl, optionally substituted C₃-C₇ cycloalkyl, optionally substituted 3-7 membered heterocyclyl, optionally substituted C₂-C₈ alkenyl, and optionally substituted C₂-C₈ alkynyl; (preferably, R¹ is selected from iso-propyl or tert-butyl; and R² is selected from hydrogen or methyl);
R³ is hydrogen, optionally substituted C(O)C₁-C₈ alkyl, optionally substituted C(O)C₁-C₈alkoxyC₁-C₈alkyl, optionally substituted C(O)C₁-C₈ haloalkyl, optionally substituted C(O)C₁-C₈ hydroxyalkyl, optionally substituted C(O)C₁-C₈ aminoalkyl, optionally substituted C(O)C₁-C₈alkylaminoC₁-C₈alkyl, optionally substituted C(O)C₃-C₇ cycloalkyl, optionally substituted C(O)(3-7 membered heterocyclyl), optionally substituted C(O)C₂-C₈ alkenyl, optionally substituted C(O)C₂-C₈ alkynyl, optionally substituted C(O)OC₁-C₈alkoxyC₁-C₈alkyl, optionally substituted C(O)OC₁-C₈ haloalkyl, optionally substituted C(O)OC₁-C₈ hydroxyalkyl, optionally substituted C(O)OC₁-C₈ aminoalkyl, optionally substituted C(O)OC₁-C₈alkylaminoC₁-C₈alkyl, optionally substituted C(O)OC₃-C₇ cycloalkyl, optionally substituted C(O)O(3-7 membered heterocyclyl), optionally substituted C(O)OC₂-C₈ alkenyl, optionally substituted C(O)OC₂-C₈ alkynyl, optionally substituted C(O)NC₁-C₈alkoxyC₁-C₈alkyl, optionally substituted C(O)NC₁-C₈ haloalkyl, optionally substituted C(O)NC₁-C₈ hydroxyalkyl, optionally substituted C(O)NC₁-C₈ aminoalkyl, optionally substituted C(O)NC₁-C₈alkylaminoC₁-C₈alkyl, optionally substituted C(O)NC₃-C₇ cycloalkyl, optionally substituted C(O)N(3-7 membered heterocyclyl), optionally substituted C(O)NC₂-C₈ alkenyl, optionally substituted C(O)NC₂-C₈ alkynyl, optionally substituted P(O)(OH)₂, optionally substituted P(O)(OC₁-C₈ alkyl)₂, and optionally substituted P(O)(OC₁-C₈ aryl)₂; and

R⁴ and R⁵ are independently selected from hydrogen, COR⁶, CO₂R⁶, CONR⁶R⁷, SOR⁶, SO₂R⁶, SO₂NR⁶R⁷, optionally substituted C₁-C₈ alkyl, optionally substituted C₁-C₈alkoxyC₁-C₈alkyl, optionally substituted C₁-C₈alkylaminoC₁-C₈alkyl, optionally substituted 3-8 membered cycloalkyl, optionally substituted 3-8 membered heterocyclyl, optionally substituted aryl, and optionally substituted heteroaryl; wherein

R⁶ and R⁷ are independently selected from hydrogen, optionally substituted C₁-C₈ alkyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted 3-8 membered cycloalkyl, optionally substituted 3-8 membered heterocyclyl, optionally substituted aryl, and optionally substituted heteroaryl; or

R⁴ and R⁵; R⁶ and R⁷ together with the atom to which they are connected form a 4-8 membered cycloalkyl or heterocyclyl ring;

Ar is selected from aryl and heteroaryl, each of which is optionally substituted with one or more substituents independently selected from F, Cl, CN, NO₂, OR⁸, NR⁸R⁹, COR⁸, CO₂R⁸, CONR⁸R⁹, SOR⁸, SO₂R⁸, SO₂NR⁹R¹⁰, NR⁹COR¹⁰, NR⁸C(O)NR⁹R¹⁰, NR⁹SOR¹⁰, NR⁹SO₂R¹⁰, optionally substituted C₁-C₆ alkyl, optionally substituted C₁-C₆ alkoxyalkyl, optionally substituted C₁-C₆ haloalkyl, optionally substituted C₁-C₆ hydroxyalkyl, optionally substituted C₁-C₆alkylaminoC₁-C₆alkyl, optionally substituted C₃-C₇ cycloalkyl, optionally substituted 3-7 membered heterocyclyl, optionally substituted C₂-C₆ alkenyl, optionally substituted C₂-C₆ alkynyl, optionally substituted aryl, and optionally substituted C₄-C₈ heteroaryl; wherein

R⁸, R⁹, and R¹⁰ are independently selected from null, hydrogen, optionally substituted C₁-C₆ alkyl, optionally substituted C₂-C₆ alkenyl, optionally substituted C₂-C₆ alkynyl, optionally substituted C₃-C₇ cycloalkyl, optionally substituted 3-7 membered heterocyclyl, optionally substituted aryl, and optionally substituted heteroaryl; or

R⁸ and R⁹; R⁹ and R¹⁰ together with the atom to which they are connected form a 4-8 membered cycloalkyl or heterocyclyl ring; and

pharmaceutically acceptable salts thereof.
In an embodiment, degradation/disruption tags include a moiety according to FORMULA 14A:

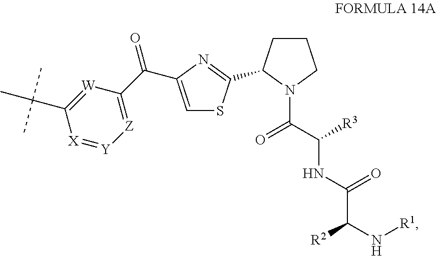

wherein
V, W, X, and Z are independently selected from CR⁴ and N;
R¹, R², R³, and R⁴ are independently selected from hydrogen, optionally substituted C₁-C₈ alkyl, optionally substituted C₁-C₈alkoxyC₁-C₈alkyl, optionally substituted C₁-C₈ haloalkyl, optionally substituted C₁-C₈ hydroxyalkyl, optionally substituted C₃-C₇ cycloalkyl, optionally substituted 3-7 membered heterocyclyl, optionally substituted C₂-C₈ alkenyl, and optionally substituted C₂-C₈ alkynyl.
In an embodiment, degradation/disruption tags include a moiety according to FORMULA 14B:

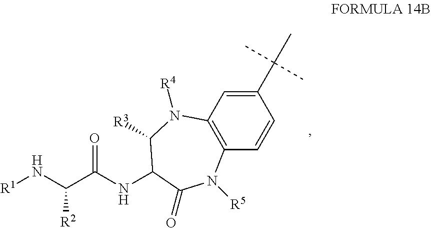

wherein
R¹, R², and R³ are independently selected from hydrogen, halogene, optionally substituted C₁-C₈ alkyl, optionally substituted C₁-C₈alkoxyC₁-C₈alkyl, optionally substituted C₁-C₈ haloalkyl, optionally substituted C₁-C₈ hydroxyalkyl, optionally substituted C₃-C₇ cycloalkyl, optionally substituted 3-7 membered heterocyclyl, optionally substituted C₂-C₈ alkenyl, and optionally substituted C₂-C₈ alkynyl;

R⁴ and R⁵ are independently selected from hydrogen, COR⁶, CO₂R⁶, CONR⁶R⁷, SOR⁶, SO₂R⁶, SO₂NR⁶R⁷, optionally substituted C₁-C₈ alkyl, optionally substituted C₁-C₈alkoxyC₁-C₈alkyl, optionally substituted C₁-C₈alkylaminoC₁-C₈alkyl, optionally substituted aryl-C₁-C₈alkyl, optionally substituted 3-8 membered cycloalkyl, optionally substituted 3-8 membered heterocyclyl, optionally substituted aryl, and optionally substituted heteroaryl; wherein

R⁶ and R⁷ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted 3-8 membered cycloalkyl, optionally substituted 3-8 membered heterocyclyl, optionally substituted aryl, and optionally substituted heteroaryl; or

R⁶ and R⁷ together with the atom to which they are connected form a 4-8 membered cycloalkyl or heterocyclyl ring; and pharmaceutically acceptable salts thereof.

In an embodiment, degradation/disruption tags are selected from the group consisting of:

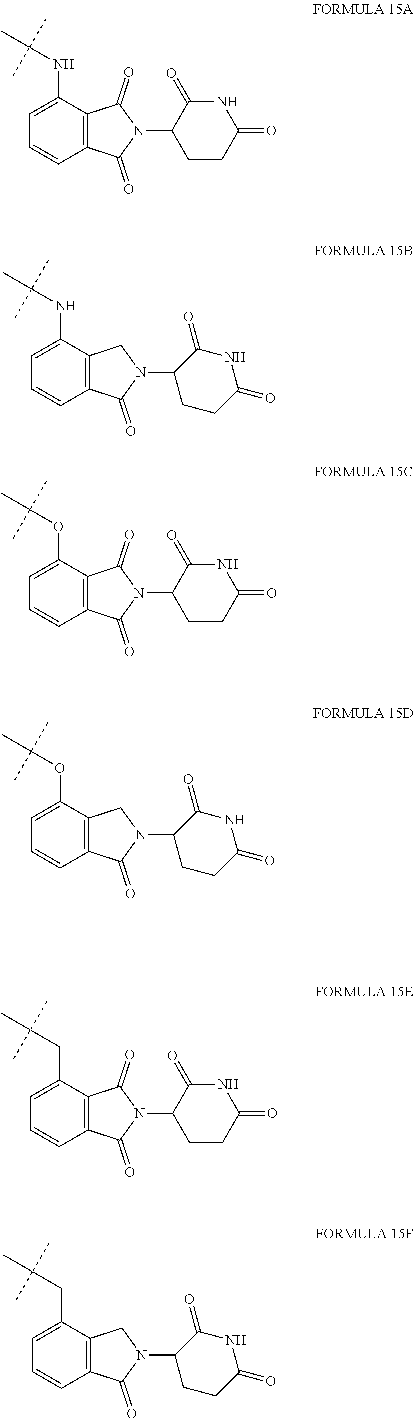

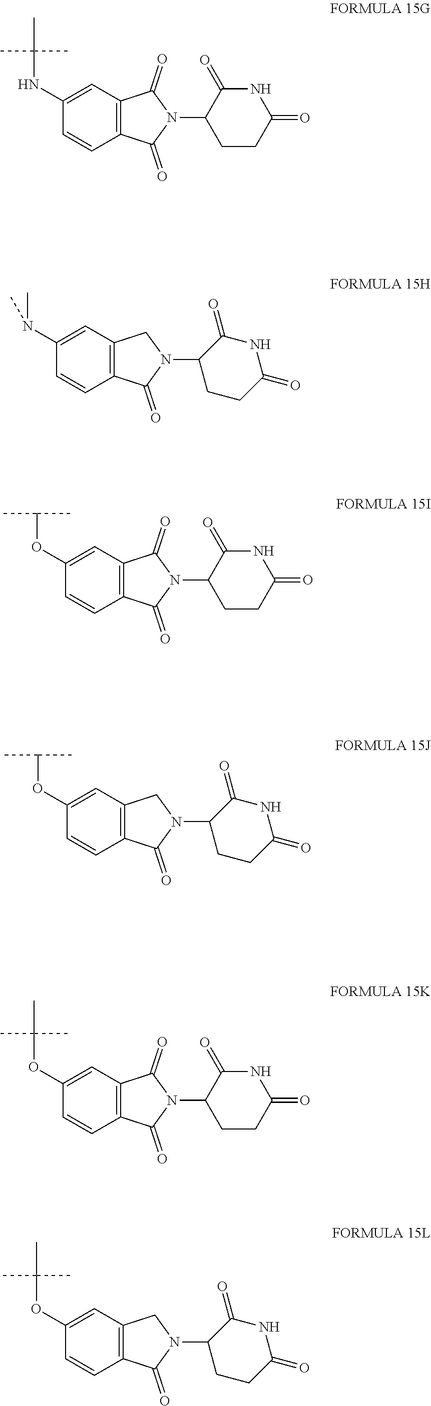

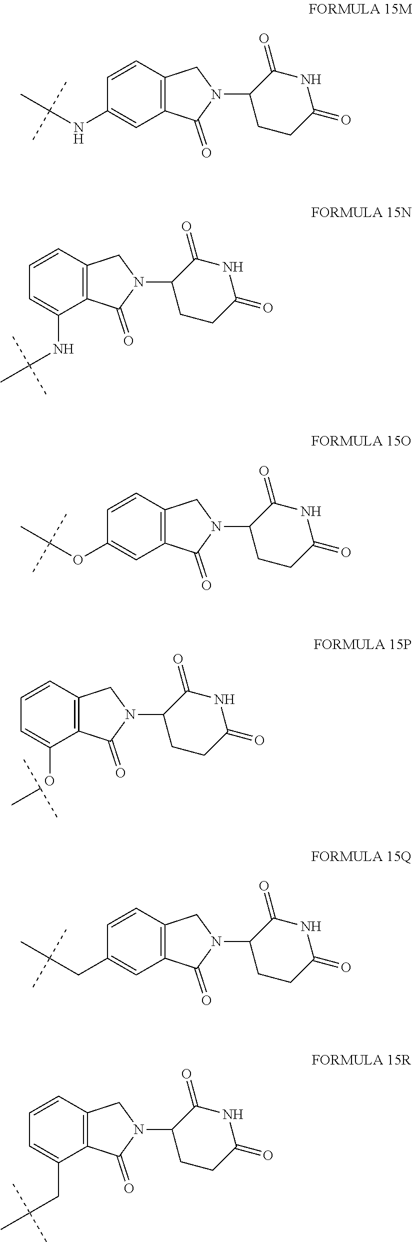

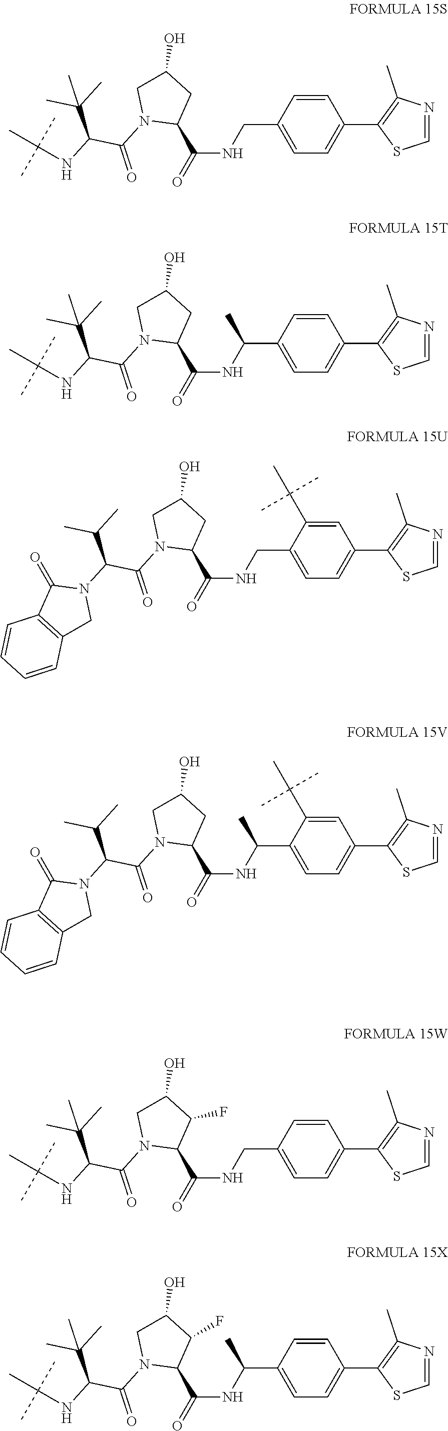

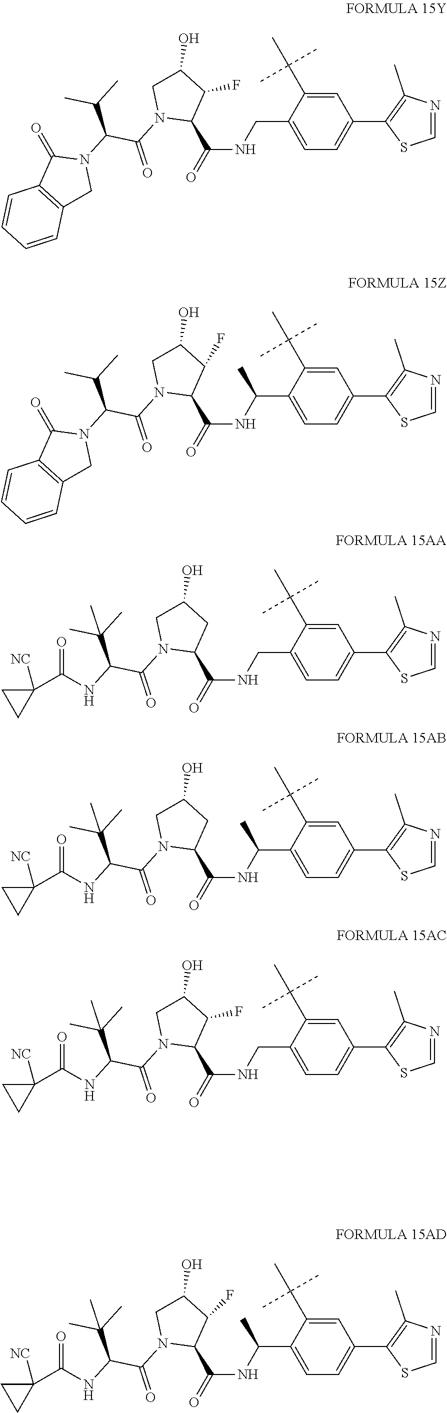

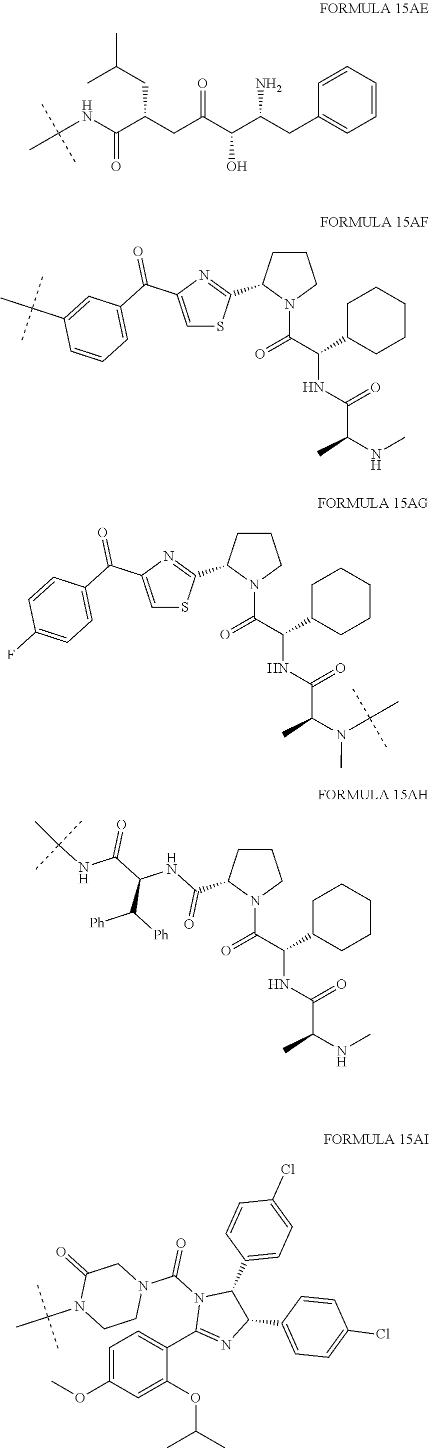

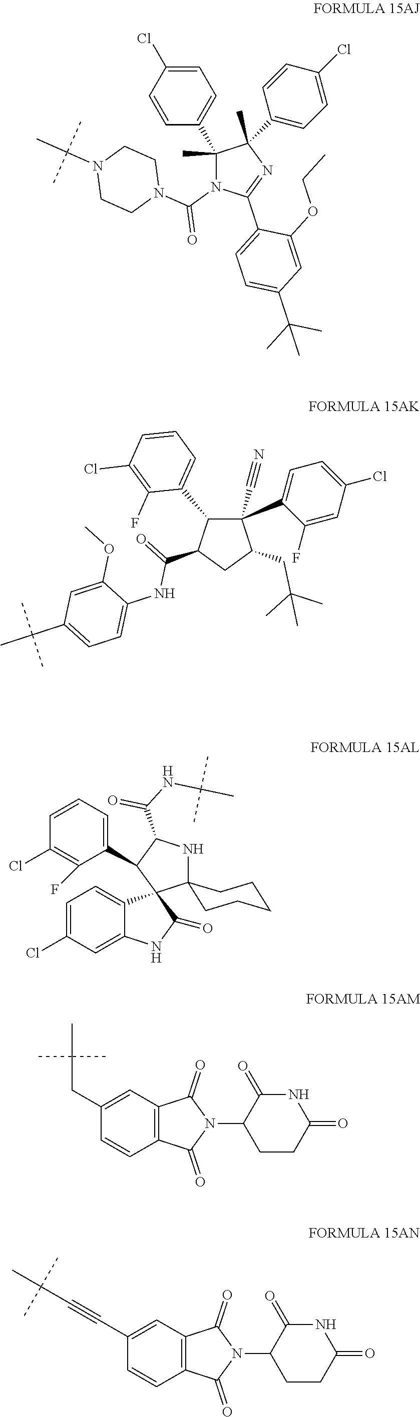

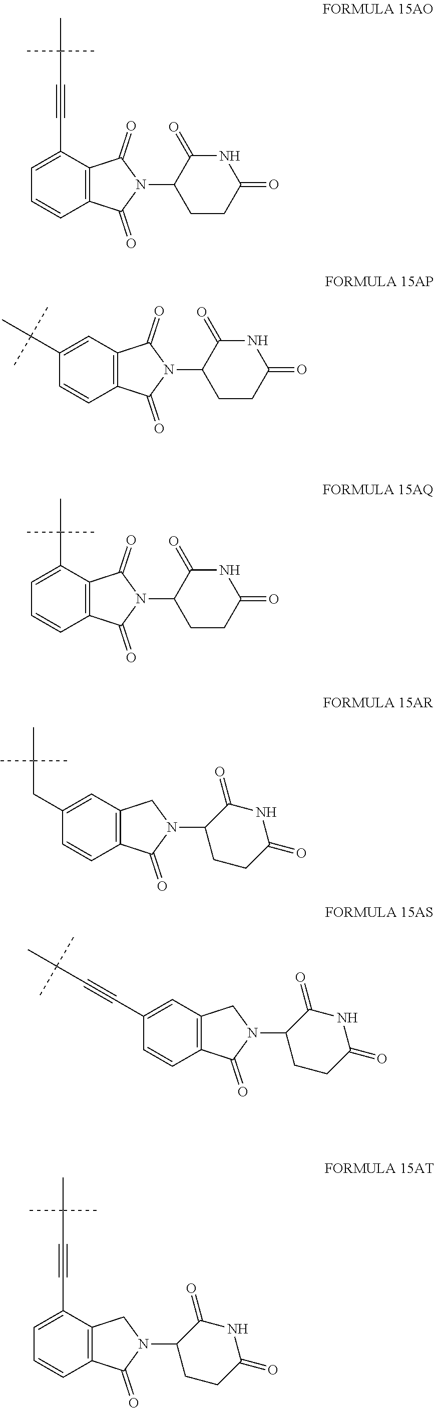

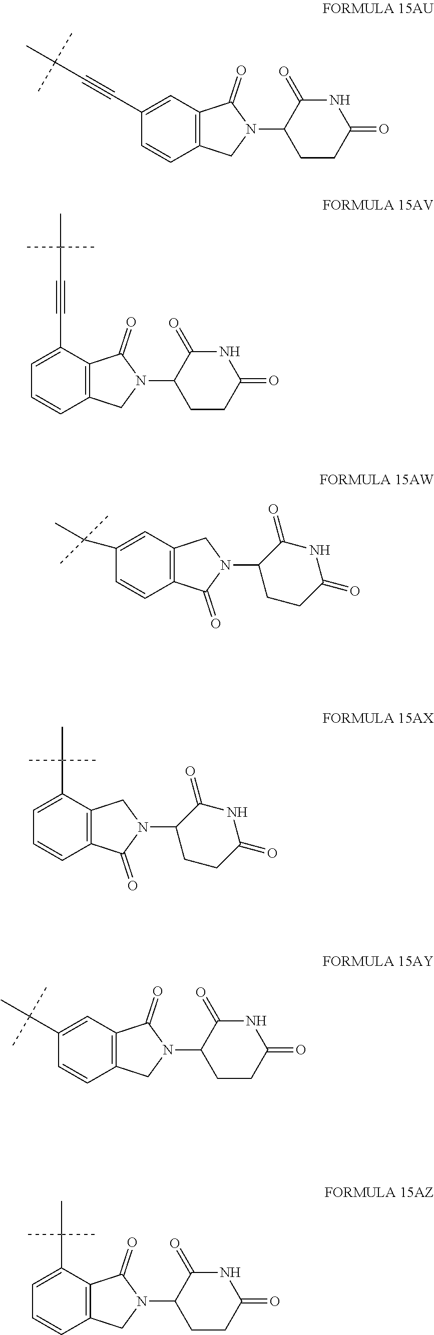

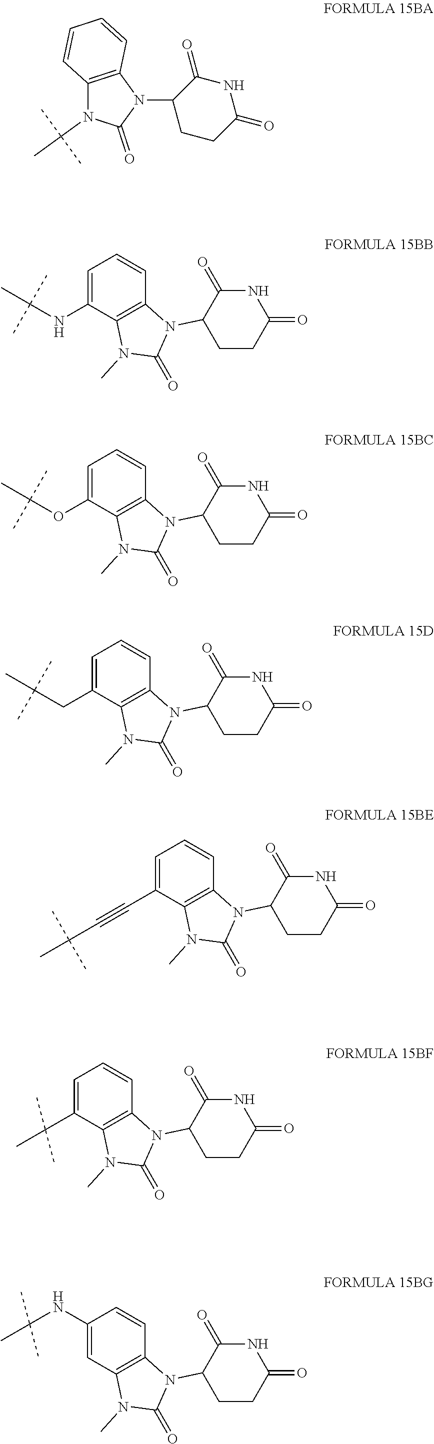

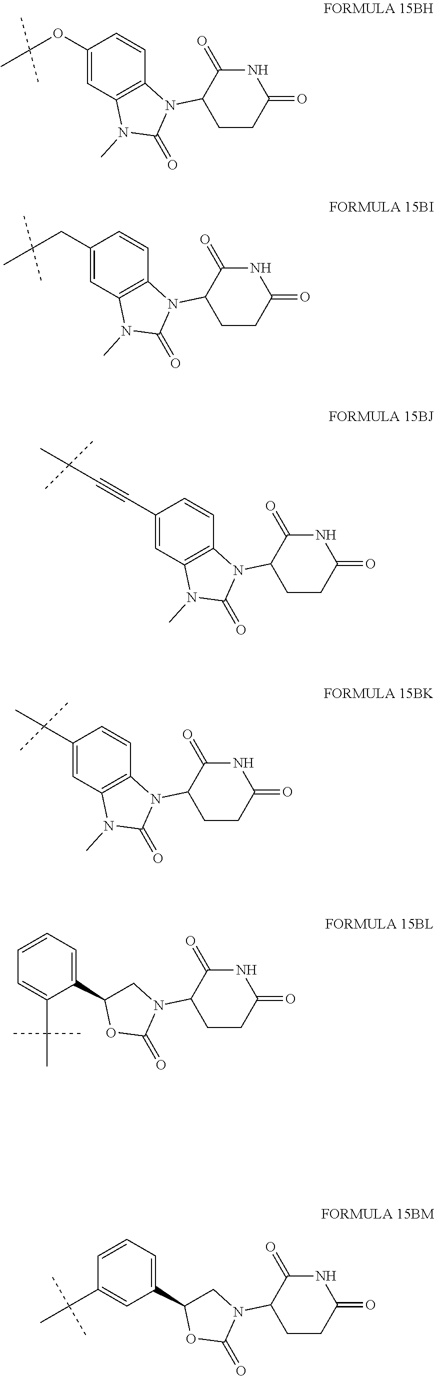

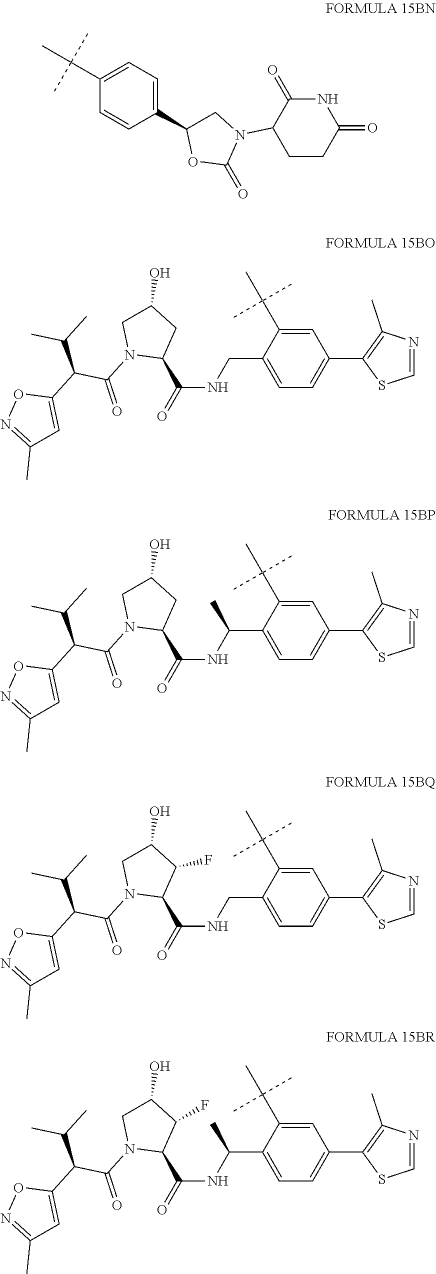

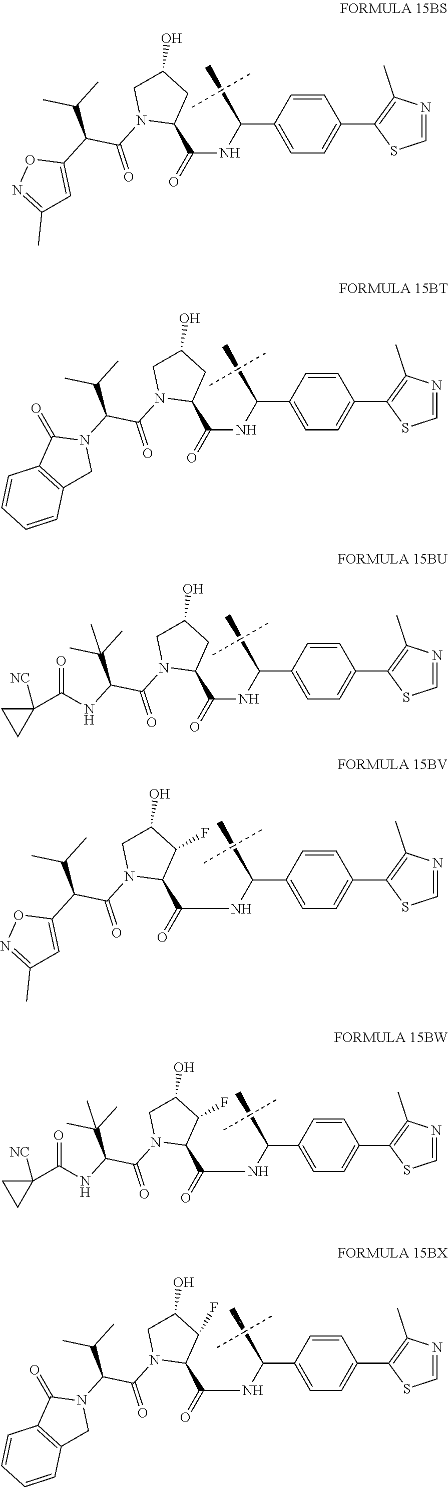

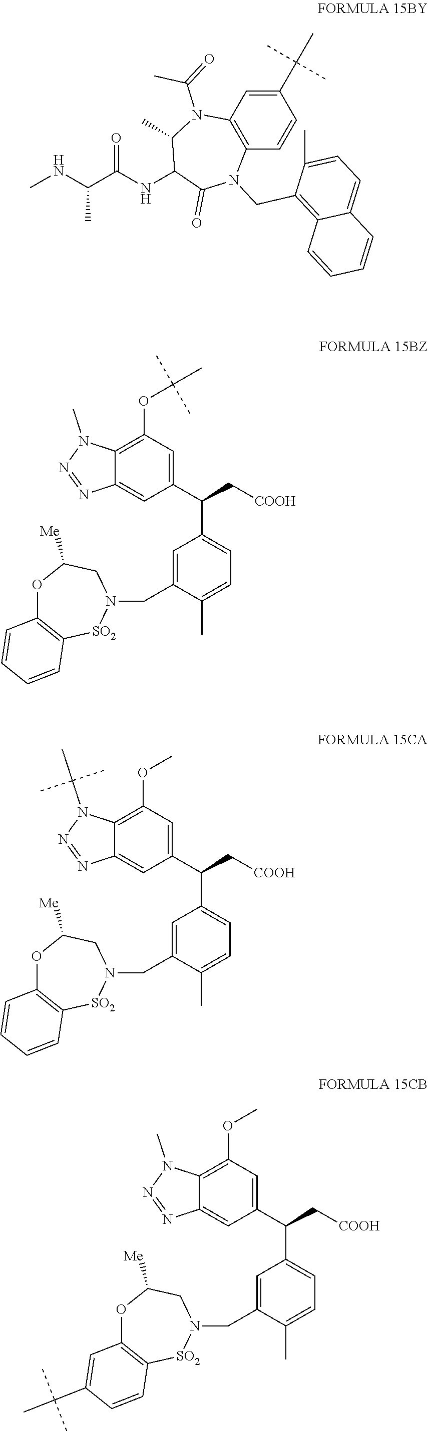

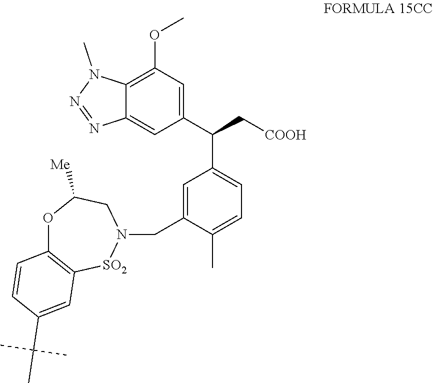

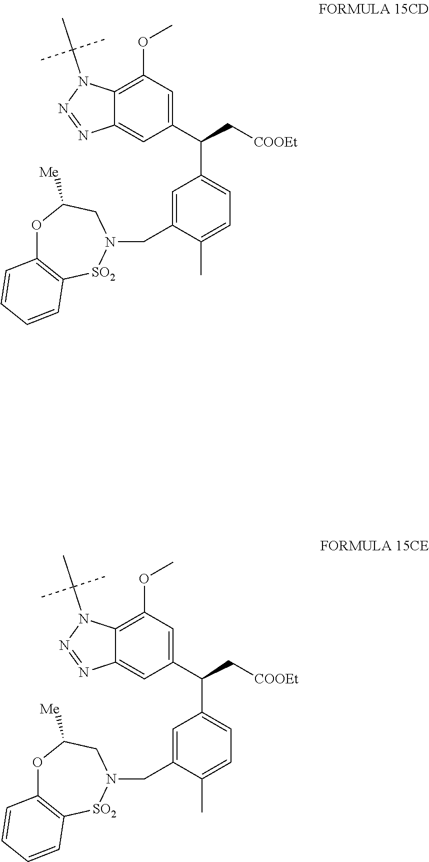

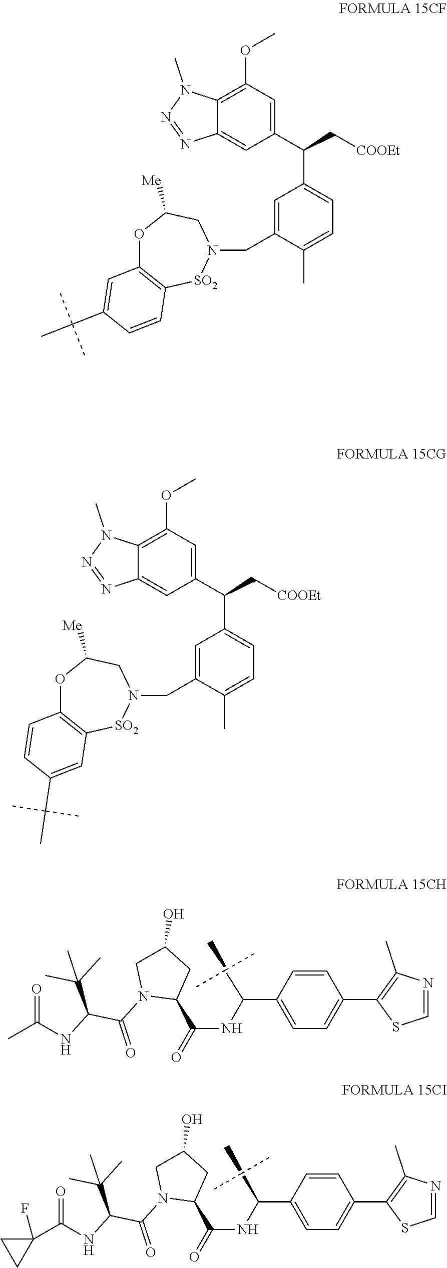

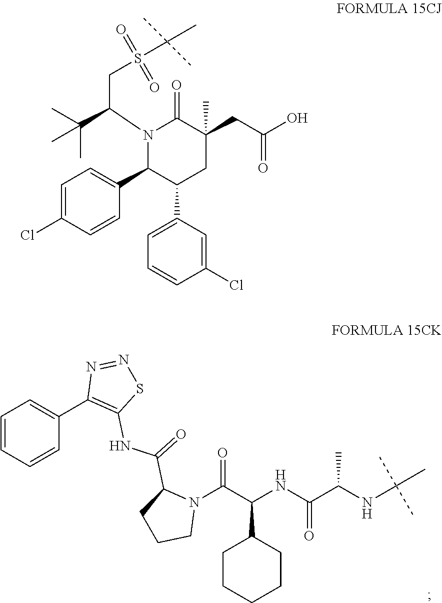

and
pharmaceutically acceptable salts thereof.

### Linkers

In any of the above-described compounds, the HPK1 ligand can be conjugated to the degradation/disruption tag through a linker. The linker can include, e.g., acyclic or cyclic saturated or unsaturated carbon, ethylene glycol, amide, amino, ether, urea, carbamate, aromatic, heteroaromatic, heterocyclic, and/or carbonyl containing groups with different lengths.
In an embodiment, the linker is a moiety according to FORMULA 16:

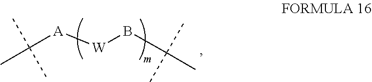

wherein
A, W, and B, at each occurrence, are independently selected from null, CO, CO₂, C(O)NR¹, C(S)NR¹, O, S, SO, SO₂, SO₂NR¹, NR¹, NR⁷CO, NR¹CONR², NR⁷C(S), optionally substituted C₁-C₈ alkyl, optionally substituted C₁-C₈ alkoxy, optionally substituted C₁-C₈alkoxyC₁-C₈alkyl, optionally substituted C₁-C₈ haloalkyl, optionally substituted C₁-C₈ hydroxyalkyl, optionally substituted C₂-C₈ alkenyl, optionally substituted C₂-C₈ alkynyl, optionally substituted 3-8 membered cycloalkyl, optionally substituted C₃-C₈ cycloalkoxy, optionally substituted 3-8 membered heterocyclyl, optionally substituted aryl, optionally substituted heteroaryl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, and optionally substituted C₃-C₁₃ spiro heterocyclyl; wherein
R¹ and R² are independently selected from hydrogen, optionally substituted C₁-C₈ alkyl, optionally substituted C₃-C₈ cycloalkyl, optionally substituted 3-8 membered cycloalkoxy, optionally substituted 3-8 membered heterocyclyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, and optionally substituted C₁-C₈alkylaminoC₁-C₈alkyl; and
m is 0 to 15.
In an embodiment, the linker is a moiety according to FORMULA 16A:

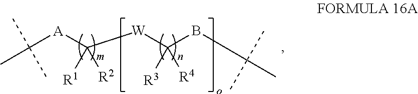

wherein
R¹, R², R³, and R⁴, at each occurrence, are independently selected from hydrogen, halogen, CN, OH, NH₂, optionally substituted C₁-C₈ alkyl, optionally substituted C₃-C₈ cycloalkyl, optionally substituted C₃-C₈ cycloalkoxy, optionally substituted 3-8 membered membered heterocyclyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, and optionally substituted C₁-C₈ alkylaminoC₁-C₈ alkyl;
A, W, and B, at each occurrence, are independently selected from null, CO, CO₂, C(O)NR⁵, C(S)NR⁵, O, S, SO, SO₂, SO₂NR⁵, NR⁵, NR⁵CO, NR⁵CONR⁶, NR⁵C(S), optionally substituted C₁-C₈ alkyl, optionally substituted C₁-C₈ alkoxy, optionally substituted C₁-C₈alkoxyC₁-C₈alkyl, optionally substituted C₁-C₈ haloalkyl, optionally substituted C₁-C₈ hydroxyalkyl, optionally substituted C₂-C₈ alkenyl, optionally substituted C₂-C₈ alkynyl, optionally substituted 3-8 membered cycloalkyl, optionally substituted C₃-C₈ cycloalkoxy, optionally substituted 3-8 membered heterocyclyl, optionally substituted aryl, optionally substituted heteroaryl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, and optionally substituted C₃-C₁₃ spiro heterocyclyl; wherein

R⁵ and R⁶ are independently selected from hydrogen, optionally substituted C₁-C₈ alkyl, optionally substituted 3-8 membered cycloalkyl, optionally substituted C₃-C₈ cycloalkoxy, optionally substituted 3-8 membered heterocyclyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, and optionally substituted C₁-C₈alkylaminoC₁-C₈alkyl;

m is 0 to 15;
n, at each occurrence, is 0 to 15;
o is 0 to 15.
In an embodiment, the linker is a moiety according to FORMULA 16B:

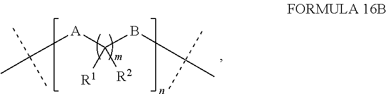

wherein
R¹ and R², at each occurrence, are independently selected from hydrogen, halogen, CN, OH, NH₂, and optionally substituted C₁-C₈ alkyl, optionally substituted 3-8 membered cycloalkyl, optionally substituted C₃-C₈ cycloalkoxy, optionally substituted 3-8 membered heterocyclyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, or C₁-C₈alkylaminoC₁-C₈alkyl;
A and B, at each occurrence, are independently selected from null, CO, CO₂, C(O)NR³, C(S)NR³, O, S, SO, SO₂, SO₂NR³, NR³, NR³CO, NR³CONR⁴, NR³C(S), and optionally substituted C₁-C₈ alkyl, optionally substituted C₁-C₈ alkoxy, optionally substituted C₁-C₈alkoxyC₁-C₈alkyl, optionally substituted C₁-C₈ haloalkyl, optionally substituted C₁-C₈ hydroxyalkyl, optionally substituted C₂-C₈ alkenyl, optionally substituted C₂-C₈ alkynyl, optionally substituted 3-8 membered cycloalkyl, optionally substituted C₃-C₈ cycloalkoxy, optionally substituted 3-8 membered heterocyclyl, optionally substituted aryl, optionally substituted heteroaryl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, or C₃-C₁₃ spiro heterocyclyl; wherein
R³ and R⁴ are independently selected from hydrogen, and optionally substituted C₁-C₈ alkyl, optionally substituted 3-8 membered cycloalkyl, optionally substituted C₃-C₈ cycloalkoxy, optionally substituted 3-8 membered heterocyclyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, or C₁-C₈alkylaminoC₁-C₈alkyl;
each m is 0 to 15; and
n is 0 to 15.
In an embodiment, the linker is a moiety according to FORMULA 16C:

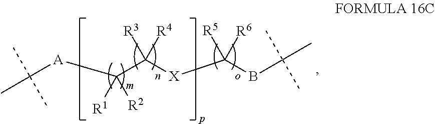

wherein
X is selected from O, NH, and NR⁷;
R¹, R², R³, R⁴, R⁵, and R⁶, at each occurrence, are independently selected from hydrogen, halogen, CN, OH, NH₂, optionally substituted C₁-C₈ alkyl, optionally substituted 3-8 membered cycloalkyl, optionally substituted C₃-C₈ cycloalkoxy, optionally substituted 3-8 membered heterocyclyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, and optionally substituted C₁-C₈ alkylaminoC₁-C₈ alkyl;
A and B, at each occurrence, are independently selected from null, CO, NH, NH—CO, CO—NH, CH₂—NH—CO, CH₂—CO—NH, NH—CO—CH₂, CO—NH—CH₂, CH₂—NH—CH₂—CO—NH, CH₂—NH—CH₂—NH—CO, —CO—NH, CO—NH—CH₂—NH—CH₂, CH₂—NH—CH₂, CO₂, C(O)NR⁷, C(S)NR⁷, O, S, SO, SO₂, SO₂NR⁷, NR⁷, NR⁷CO, NR⁷CONR⁸, NR⁷C(S), optionally substituted C₁-C₈ alkyl, optionally substituted C₁-C₈ alkoxy, optionally substituted C₁-C₈alkoxyC₁-C₈alkyl, optionally substituted C₁—C₈ haloalkyl, optionally substituted C₁-C₈ hydroxyalkyl, optionally substituted C₂-C₈ alkenyl, optionally substituted C₂-C₈ alkynyl, optionally substituted 3-8 membered cycloalkyl, optionally substituted C₃-C₈ cycloalkoxy, optionally substituted 3-8 membered heterocyclyl, optionally substituted aryl, optionally substituted heteroaryl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, and optionally substituted C₃-C₁₃ spiro heterocyclyl; wherein
R⁷ and R⁸ are independently selected from hydrogen, optionally substituted C₁-C₈ alkyl, optionally substituted 3-8 membered cycloalkyl, optionally substituted C₃-C₈ cycloalkoxy, optionally substituted 3-8 membered heterocyclyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, and optionally substituted C₁-C₈ alkylaminoC₁-C₈ alkyl;
m, at each occurrence, is 0 to 15;
n, at each occurrence, is 0 to 15;
o is 0 to 15; and
p is 0 to 15; and
pharmaceutically acceptable salts thereof.
In an embodiment, the linker is selected from the group consisting of a ring selected from the group consisting of a 3 to 13 membered ring; a 3 to 13 membered fused ring; a 3 to 13 membered bridged ring; and a 3 to 13 membered spiro ring; and pharmaceutically acceptable salts thereof.
In an embodiment, the linker is a moiety according to one of FORMULAE C1, C2, C3, C4 and C5:

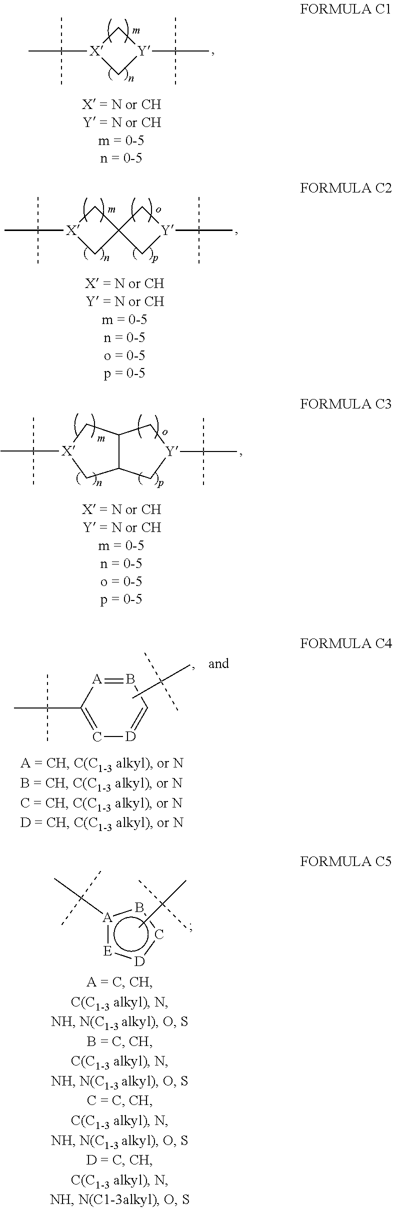

and pharmaceutically acceptable salts thereof.

### Synthesis and Testing of Bivalent Compounds

The binding affinity of novel synthesized bivalent compounds (i.e., HPK1 degraders/disruptors) can be assessed using standard biophysical assays known in the art (e.g., isothermal titration calorimetry (ITC)). Cellular assays can then be used to assess the bivalent compound's ability to induce HPK1 degradation and inhibit cancer cell proliferation. Besides evaluating bivalent compound's-induced changes in the protein expression of HPK1, enzymatic activity can also be assessed. Assays suitable for use in any or all of these steps are known in the art, and include, e.g., Western blotting, quantitative mass spectrometry (MS) analysis, flow cytometry, enzymatic inhibition, ITC, SPR, cell growth inhibition and xenograft and PDX models. Suitable cell lines for use in any or all of these steps are known in the art and include, e.g., HPK1-deficient Jurkat clone that had been created by CRISPR-mediated frameshift mutation that resulted in the loss of HPK1 expression in Jurkat T cells. Such line could serve as a platform for counter screening the lead HPK1 degraders/disruptors for non-HPK1-specific effects. By way of non-limiting example, detailed synthesis protocols are described in the Examples for specific exemplary HPK1 degraders/disruptors.

Pharmaceutically acceptable isotopic variations of the compounds disclosed herein are contemplated and can be synthesized using conventional methods known in the art or methods corresponding to those described in the Examples (substituting appropriate reagents with appropriate isotopic variations of those reagents). Specifically, an isotopic variation is a compound in which at least one atom is replaced by an atom having the same atomic number, but an atomic mass different from the atomic mass usually found in nature. Useful isotopes are known in the art and include, for example, isotopes of hydrogen, carbon, nitrogen, oxygen, phosphorus, sulfur, fluorine, and chlorine. Exemplary isotopes thus include, e.g., ²H, ³H, ¹³C, ¹⁴C, ¹⁵N, ¹⁷O, ¹⁸O, ³²P, ³⁵S, ¹⁸F, and ³⁶Cl.

Isotopic variations (e.g., isotopic variations containing ²H) can provide therapeutic advantages resulting from greater metabolic stability, e.g., increased in vivo half-life or reduced dosage requirements. In addition, certain isotopic variations (particularly those containing a radioactive isotope) can be used in drug or substrate tissue distribution studies. The radioactive isotopes tritium (3H) and carbon-14 (¹⁴C) are particularly useful for this purpose in view of their ease of incorporation and ready means of detection.

Pharmaceutically acceptable solvates of the compounds disclosed herein are contemplated. A solvate can be generated, e.g., by substituting a solvent used to crystallize a compound disclosed herein with an isotopic variation (e.g., D₂O in place of H₂O, d₆-acetone in place of acetone, or d₆-DMSO in place of DMSO).

Pharmaceutically acceptable fluorinated variations of the compounds disclosed herein are contemplated and can be synthesized using conventional methods known in the art or methods corresponding to those described in the Examples (substituting appropriate reagents with appropriate fluorinated variations of those reagents). Specifically, a fluorinated variation is a compound in which at least one hydrogen atom is replaced by a fluoro atom. Fluorinated variations can provide therapeutic advantages resulting from greater metabolic stability, e.g., increased in vivo half-life or reduced dosage requirements

### Definition of Terms

As used herein, the terms “comprising” and “including” are used in their open, non-limiting sense.

“Alkyl” refers to a straight or branched hydrocarbon chain radical consisting solely of carbon and hydrogen atoms, containing no unsaturation. An alkyl may comprise one, two, three, four, five, six, seven, eight, nine, ten, eleven, twelve, thirteen, fourteen, fifteen, or sixteen carbon atoms. In certain embodiments, an alkyl comprises one to fifteen carbon atoms (e.g., C₁-C₁₅ alkyl). In certain embodiments, an alkyl comprises one to thirteen carbon atoms (e.g., C₁-C₁₃ alkyl). In certain embodiments, an alkyl comprises one to eight carbon atoms (e.g., C₁-C₈ alkyl). In other embodiments, an alkyl comprises five to fifteen carbon atoms (e.g., C₅-C₁₅ alkyl). In other embodiments, an alkyl comprises five to eight carbon atoms (e.g., C₅-C₈ alkyl). The alkyl is attached to the rest of the molecule by a single bond, for example, methyl (Me), ethyl (Et), n-propyl, 1-methylethyl (iso-propyl), n-butyl, n-pentyl, 1,1-dimethylethyl (t-butyl), pentyl, 3-methylhexyl, 2-methylhexyl, and the like.

“Alkenyl” refers to a straight or branched hydrocarbon chain radical group consisting solely of carbon and hydrogen atoms, containing at least one double bond. An alkenyl may comprise two, three, four, five, six, seven, eight, nine, ten, eleven, twelve, thirteen, fourteen, fifteen, or sixteen carbon atoms. In certain embodiments, an alkenyl comprises two to twelve carbon atoms (e.g., C₂-C₁₂ alkenyl). In certain embodiments, an alkenyl comprises two to eight carbon atoms (e.g., C₂-C₈ alkenyl). In certain embodiments, an alkenyl comprises two to six carbon atoms (e.g., C₂-C₆ alkenyl). In other embodiments, an alkenyl comprises two to four carbon atoms (e.g., C₂-C₄ alkenyl). The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like.

The term “allyl,” as used herein, means a —CH₂CH═CH₂ group.

As used herein, the term “alkynyl” refers to a straight or branched hydrocarbon chain radical group consisting solely of carbon and hydrogen atoms, containing at least one triple bond. An alkynyl may comprise two, three, four, five, six, seven, eight, nine, ten, eleven, twelve, thirteen, fourteen, fifteen, or sixteen carbon atoms. In certain embodiments, an alkynyl comprises two to twelve carbon atoms (e.g., C₂-C₁₂ alkynyl). In certain embodiments, an alkynyl comprises two to eight carbon atoms (e.g., C₂-C₈ alkynyl). In other embodiments, an alkynyl has two to six carbon atoms (e.g., C₂-C₆ alkynyl). In other embodiments, an alkynyl has two to four carbon atoms (e.g., C₂-C₄ alkynyl). The alkynyl is attached to the rest of the molecule by a single bond. Examples of such groups include, but are not limited to, ethynyl, propynyl, 1-butynyl, 2-butynyl, 1-pentynyl, 2-pentynyl, 1-hexynyl, 2-hexynyl, 3-hexynyl, and the like.

The term “alkoxy”, as used herein, means an alkyl group as defined herein which is attached to the rest of the molecule via an oxygen atom. Examples of such groups include, but are not limited to, methoxy, ethoxy, n-propyloxy, iso-propyloxy, n-butoxy, iso-butoxy, tert-butoxy, pentyloxy, hexyloxy, and the like.

The term “aryl”, as used herein, “refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon atoms. An aryl may comprise from six to eighteen carbon atoms, where at least one of the rings in the ring system is fully unsaturated, i.e., it contains a cyclic, delocalized (4n+2) π-electron system in accordance with the Hückel theory. In certain embodiments, an aryl comprises six to fourteen carbon atoms (C₆-C₁₄ aryl). In certain embodiments, an aryl comprises six to ten carbon atoms (C₆-C₁₀ aryl). Examples of such groups include, but are not limited to, phenyl, fluorenyl and naphthyl. The terms “Ph” and “phenyl,” as used herein, mean a —C₆H₅ group.

The term “heteroaryl”, refers to a radical derived from a 3- to 18-membered aromatic ring radical that comprises two to seventeen carbon atoms and from one to six heteroatoms selected from nitrogen, oxygen and sulfur. As used herein, the heteroaryl radical may be a monocyclic, bicyclic, tricyclic or tetracyclic ring system, wherein at least one of the rings in the ring system is fully unsaturated, i.e., it contains a cyclic, delocalized (4n+2) π-electron system in accordance with the Hückel theory. Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Examples of such groups include, but not limited to, pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, furopyridinyl, and the like. In certain embodiments, an heteroaryl is attached to the rest of the molecule via a ring carbon atom. In certain embodiments, an heteroaryl is attached to the rest of the molecule via a nitrogen atom (N-attached) or a carbon atom (C-attached). For instance, a group derived from pyrrole may be pyrrol-1-yl (N-attached) or pyrrol-3-yl (C-attached). Further, a group derived from imidazole may be imidazol-1-yl (N-attached) or imidazol-3-yl (C-attached).

The term “heterocyclyl”, as used herein, means a non-aromatic, monocyclic, bicyclic, tricyclic, or tetracyclic radical having a total of from 4, 5, 6, 7, 8, 9, 10, 11, 12, or 13 atoms in its ring system, and containing from 3 to 12 carbon atoms and from 1 to 4 heteroatoms each independently selected from O, S and N, and with the proviso that the ring of said group does not contain two adjacent O atoms or two adjacent S atoms. A heterocyclyl group may include fused, bridged or spirocyclic ring systems. In certain embodiments, a heterocyclyl group comprises 3 to ring atoms (3-10 membered heterocyclyl). In certain embodiments, a heterocyclyl group comprises 3 to 8 ring atoms (3-8 membered heterocyclyl). In certain embodiments, a heterocyclyl group comprises 4 to 8 ring atoms (4-8 membered heterocyclyl). In certain embodiments, a heterocyclyl group comprises 3 to 6 ring atoms (3-6 membered heterocyclyl). A heterocyclyl group may contain an oxo substituent at any available atom that will result in a stable compound. For example, such a group may contain an oxo atom at an available carbon or nitrogen atom. Such a group may contain more than one oxo substituent if chemically feasible. In addition, it is to be understood that when such a heterocyclyl group contains a sulfur atom, said sulfur atom may be oxidized with one or two oxygen atoms to afford either a sulfoxide or sulfone. An example of a 4 membered heterocyclyl group is azetidinyl (derived from azetidine). An example of a 5 membered cycloheteroalkyl group is pyrrolidinyl. An example of a 6 membered cycloheteroalkyl group is piperidinyl. An example of a 9 membered cycloheteroalkyl group is indolinyl. An example of a 10 membered cycloheteroalkyl group is 4H-quinolizinyl. Further examples of such heterocyclyl groups include, but are not limited to, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl, quinolizinyl, 3-oxopiperazinyl, 4-methylpiperazinyl, 4-ethylpiperazinyl, and 1-oxo-2,8,diazaspiro[4.5]dec-8-yl. A heteroaryl group may be attached to the rest of molecular via a carbon atom (C-attached) or a nitrogen atom (N-attached). For instance, a group derived from piperazine may be piperazin-1-yl (N-attached) or piperazin-2-yl (C-attached).

The term “cycloalkyl” means a saturated, monocyclic, bicyclic, tricyclic, or tetracyclic radical having a total of from 4, 5, 6, 7, 8, 9, 10, 11, 12, or 13 carbon atoms in its ring system. A cycloalkyl may be fused, bridged or spirocyclic. In certain embodiments, a cycloalkyl comprises 3 to 8 carbon ring atoms (C₃-C₈ cycloalkyl). In certain embodiments, a cycloalkyl comprises 3 to 6 carbon ring atoms (C₃-C₆ cycloalkyl). Examples of such groups include, but are not limited to, cyclopropyl, cyclobutyl, cyclopentyl, cyclopentenyl, cyclohexyl, cycloheptyl, adamantyl, and the like.

The term “cycloalkylene” is a bidentate radical obtained by removing a hydrogen atom from a cycloalkyl ring as defined above. Examples of such groups include, but are not limited to, cyclopropylene, cyclobutylene, cyclopentylene, cyclopentenylene, cyclohexylene, cycloheptylene, and the like.

The term “spirocyclic” as used herein has its conventional meaning, that is, any ring system containing two or more rings wherein two of the rings have one ring carbon in common. Each ring of the spirocyclic ring system, as herein defined, independently comprises 3 to 20 ring atoms. Preferably, they have 3 to 10 ring atoms. Non-limiting examples of a spirocyclic system include spiro[3.3]heptane, spiro[3.4]octane, and spiro[4.5]decane.

The term cyano” refers to a —C—N group.

An “aldehyde” group refers to a —C(O)H group.

An “alkoxy” group refers to both an —O-alkyl, as defined herein.

An “alkoxycarbonyl” refers to a —C(O)-alkoxy, as defined herein.

An “alkylaminoalkyl” group refers to an -alkyl-NR-alkyl group, as defined herein.

An “alkylsulfonyl” group refer to a —SO₂alkyl, as defined herein.

An “amino” group refers to an optionally substituted —NH₂.

An “aminoalkyl” group refers to an -alky-amino group, as defined herein.

An “aminocarbonyl” refers to a —C(O)-amino, as defined herein.

An “arylalkyl” group refers to -alkylaryl, where alkyl and aryl are defined herein.

An “aryloxy” group refers to both an —O-aryl and an —O-heteroaryl group, as defined herein.

An “aryloxycarbonyl” refers to —C(O)-aryloxy, as defined herein.

An “arylsulfonyl” group refers to a —SO₂aryl, as defined herein.

A “carbonyl” group refers to a —C(O)— group, as defined herein.

A “carboxylic acid” group refers to a —C(O)OH group.

A “cycloalkoxy” refers to a —O-cycloalkyl group, as defined herein.

A “halo” or “halogen” group refers to fluorine, chlorine, bromine or iodine.

A “haloalkyl” group refers to an alkyl group substituted with one or more halogen atoms.

A “hydroxy” group refers to an —OH group.

A “nitro” group refers to a —NO₂ group.

An “oxo” group refers to the ═O substituent.

A “trihalomethyl” group refers to a methyl substituted with three halogen atoms.

The term “substituted,” means that the specified group or moiety bears one or more substituents independently selected from C₁-C₄ alkyl, aryl, heteroaryl, aryl-C₁-C₄ alkyl-, heteroaryl-C₁-C₄ alkyl-, C₁-C₄ haloalkyl, —OC₁-C₄ alkyl, —OC₁-C₄ alkylphenyl, —C₁-C₄ alkyl-OH, —OC₁-C₄ haloalkyl, halo, —OH, —NH₂, —C₁-C₄ alkyl-NH₂, —N(C₁-C₄ alkyl)(C₁-C₄ alkyl), —NH(C₁-C₄ alkyl), —N(C₁-C₄ alkyl)(C₁-C₄ alkylphenyl), —NH(C₁-C₄ alkylphenyl), cyano, nitro, oxo, —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —NHC(O)(phenyl), —N(C₁-C₄ alkyl)C(O)(C₁-C₄ alkyl), —N(C₁-C₄ alkyl)C(O)(phenyl), —C(O)C₁-C₄ alkyl, —C(O)C₁-C₄ alkylphenyl, —C(O)C₁-C₄ haloalkyl, —OC(O)C₁-C₄ alkyl, —SO₂(C₁-C₄ alkyl), —SO₂(phenyl), —SO₂(C₁-C₄ haloalkyl), —SO₂NH₂, —SO₂NH(C₁-C₄ alkyl), —SO₂NH(phenyl), —NHSO₂(C₁-C₄ alkyl), —NHSO₂(phenyl), and —NHSO₂(C₁-C₄ haloalkyl).

The term “optionally substituted” means that the specified group may be either unsubstituted or substituted by one or more substituents as defined herein. It is to be understood that in the compounds of the present invention when a group is said to be “unsubstituted,” or is “substituted” with fewer groups than would fill the valencies of all the atoms in the compound, the remaining valencies on such a group are filled by hydrogen. For example, if a C₆ aryl group, also called “phenyl” herein, is substituted with one additional substituent, one of ordinary skill in the art would understand that such a group has 4 open positions left on carbon atoms of the C₆ aryl ring (6 initial positions, minus one at which the remainder of the compound of the present invention is attached to and an additional substituent, remaining 4 positions open). In such cases, the remaining 4 carbon atoms are each bound to one hydrogen atom to fill their valencies. Similarly, if a C₆ aryl group in the present compounds is said to be “disubstituted,” one of ordinary skill in the art would understand it to mean that the C₆ aryl has 3 carbon atoms remaining that are unsubstituted. Those three unsubstituted carbon atoms are each bound to one hydrogen atom to fill their valencies.

“Pharmaceutically acceptable salt” includes both acid and base addition salts. A pharmaceutically acceptable salt of any one of the bivalent compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and. aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997), which is hereby incorporated by reference in its entirety). Acid addition salts of basic compounds may be prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts may be formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

### Pharmaceutical Compositions

In some aspects, the compositions and methods described herein include the manufacture and use of pharmaceutical compositions and medicaments that include one or more bivalent compounds as disclosed herein. Also included are the pharmaceutical compositions themselves.

In some aspects, the compositions disclosed herein can include other compounds, drugs, or agents used for the treatment of cancer. For example, in some instances, pharmaceutical compositions disclosed herein can be combined with one or more (e.g., one, two, three, four, five, or less than ten) compounds. Such additional compounds can include, e.g., conventional chemotherapeutic agents known in the art. When co-administered, HPK1 degraders/disruptors disclosed herein can operate in conjunction with conventional chemotherapeutic agents to produce mechanistically additive or synergistic therapeutic effects.

In some aspects, the pH of the compositions disclosed herein can be adjusted with pharmaceutically acceptable acids, bases, or buffers to enhance the stability of the HPK1 degraders/disruptor or its delivery form.

Pharmaceutical compositions typically include a pharmaceutically acceptable carrier, adjuvant, or vehicle. As used herein, the phrase “pharmaceutically acceptable” refers to molecular entities and compositions that are generally believed to be physiologically tolerable and do not typically produce an allergic or similar untoward reaction, such as gastric upset, dizziness and the like, when administered to a human. A pharmaceutically acceptable carrier, adjuvant, or vehicle is a composition that can be administered to a patient, together with a compound of the invention, and which does not destroy the pharmacological activity thereof and is nontoxic when administered in doses sufficient to deliver a therapeutic amount of the compound. Exemplary conventional nontoxic pharmaceutically acceptable carriers, adjuvants, and vehicles include saline, solvents, dispersion media, coatings, antibacterial and antifungal agents, isotonic and absorption delaying agents, and the like, compatible with pharmaceutical administration.

In particular, pharmaceutically acceptable carriers, adjuvants, and vehicles that can be used in the pharmaceutical compositions of this invention include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, may also be advantageously used to enhance delivery of compounds of the formulae described herein.

As used herein, the HPK1 degraders/disruptors disclosed herein are defined to include pharmaceutically acceptable derivatives or prodrugs thereof. A “pharmaceutically acceptable derivative” means any pharmaceutically acceptable salt, solvate, or prodrug, e.g., carbamate, ester, phosphate ester, salt of an ester, or other derivative of a compound or agent disclosed herein, which upon administration to a recipient is capable of providing (directly or indirectly) a compound described herein, or an active metabolite or residue thereof. Particularly favored derivatives and prodrugs are those that increase the bioavailability of the compounds disclosed herein when such compounds are administered to a mammal (e.g., by allowing an orally administered compound to be more readily absorbed into the blood) or which enhance delivery of the parent compound to a biological compartment (e.g., the brain or lymphatic system) relative to the parent species. Preferred prodrugs include derivatives where a group that enhances aqueous solubility or active transport through the gut membrane is appended to the structure of formulae described herein. Such derivatives are recognizable to those skilled in the art without undue experimentation. Nevertheless, reference is made to the teaching of Burger's Medicinal Chemistry and Drug Discovery, 5^{th} Edition, Vol. 1: Principles and Practice, which is incorporated herein by reference to the extent of teaching such derivatives.

The HPK1 degraders/disruptors disclosed herein include pure enantiomers, mixtures of enantiomers, pure diastereoisomers, mixtures of diastereoisomers, diastereoisomeric racemates, mixtures of diastereoisomeric racemates and the meso-form and pharmaceutically acceptable salts, solvent complexes, morphological forms, or deuterated derivative thereof.

In particular, pharmaceutically acceptable salts of the HPK1 degraders/disruptors disclosed herein include, e.g., those derived from pharmaceutically acceptable inorganic and organic acids and bases. Examples of suitable acid salts include acetate, adipate, benzoate, benzenesulfonate, butyrate, citrate, digluconate, dodecylsulfate, formate, fumarate, glycolate, hemisulfate, heptanoate, hexanoate, hydrochloride, hydrobromide, hydroiodide, lactate, maleate, malonate, methanesulfonate, 2-naphthalenesulfonate, nicotinate, nitrate, palmoate, phosphate, picrate, pivalate, propionate, salicylate, succinate, sulfate, tartrate, tosylate, trifluoromethylsulfonate, and undecanoate. Salts derived from appropriate bases include, e.g., HPK1 alkali metal (e.g., sodium), HPK1 alkaline earth metal (e.g., magnesium), ammonium and N—(HPK1yl)4+ salts. The invention also envisions the quaternization of any basic nitrogen-containing groups of the HPK1 degraders/disruptors disclosed herein. Water or oil-soluble or dispersible products can be obtained by such quaternization.

In some aspects, the pharmaceutical compositions disclosed herein can include an effective amount of one or more HPK1 degraders/disruptors. The terms “effective amount” and “effective to treat,” as used herein, refer to an amount or a concentration of one or more compounds or a pharmaceutical composition described herein utilized for a period of time (including acute or chronic administration and periodic or continuous administration) that is effective within the context of its administration for causing an intended effect or physiological outcome (e.g., treatment or prevention of cell growth, cell proliferation, or cancer). In some aspects, pharmaceutical compositions can further include one or more additional compounds, drugs, or agents used for the treatment of cancer (e.g., conventional chemotherapeutic agents) in amounts effective for causing an intended effect or physiological outcome (e.g., treatment or prevention of cell growth, cell proliferation, or cancer).

In some aspects, the pharmaceutical compositions disclosed herein can be formulated for sale in the United States, import into the United States, or export from the United States.

### Administration of Pharmaceutical Compositions

The pharmaceutical compositions disclosed herein can be formulated or adapted for administration to a subject via any route, e.g., any route approved by the Food and Drug Administration (FDA). Exemplary methods are described in the FDA Data Standards Manual (DSM) (available at http://www.fda.gov/Drugs/DevelopmentApprovalProcess/FormsSubmissionRequirements/ElectronicSubmissions/DataStandardsManualmonographs). In particular, the pharmaceutical compositions can be formulated for and administered via oral, parenteral, or transdermal delivery. The term “parenteral” as used herein includes subcutaneous, intracutaneous, intravenous, intramuscular, intraperitoneal, intra-articular, intra-arterial, intrasynovial, intrasternal, intrathecal, intralesional, and intracranial injection or infusion techniques.

For example, the pharmaceutical compositions disclosed herein can be administered, e.g., topically, rectally, nasally (e.g., by inhalation spray or nebulizer), buccally, vaginally, subdermally (e.g., by injection or via an implanted reservoir), or ophthalmically.

For example, pharmaceutical compositions of this invention can be orally administered in any orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions or emulsions are administered orally, the active ingredient may be suspended or dissolved in an oily phase is combined with emulsifying or suspending agents. If desired, certain sweetening, flavoring, or coloring agents can be added.

For example, the pharmaceutical compositions of this invention can be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax, and polyethylene glycols.

For example, the pharmaceutical compositions of this invention can be administered by nasal aerosol or inhalation. Such compositions are prepared according to techniques well-known in the art of pharmaceutical formulation and can be prepared as solutions in saline, employing benzyl alcohol or other suitable preservatives, absorption promoters to enhance bioavailability, fluorocarbons, or other solubilizing or dispersing agents known in the art.

For example, the pharmaceutical compositions of this invention can be administered by injection (e.g., as a solution or powder). Such compositions can be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, e.g., as a solution in 1,3-butanediol. Among the acceptable vehicles and solvents that may be employed are mannitol, water, Ringer's solution, and isotonic sodium chloride solution. In addition, sterile, fixed oils are conventionally employed as a solvent or suspending medium. For this purpose, any bland fixed oil can be employed, including synthetic mono- or diglycerides. Fatty acids, such as oleic acid and its glyceride derivatives are useful in the preparation of injectables, as are natural pharmaceutically-acceptable oils, e.g., olive oil or castor oil, especially in their polyoxyethylated versions. These oil solutions or suspensions can also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens, Spans, or other similar emulsifying agents or bioavailability enhancers which are commonly used in the manufacture of pharmaceutically acceptable solid, liquid, or other dosage forms can also be used for the purposes of formulation.

In some aspects, an effective dose of a pharmaceutical composition of this invention can include, but is not limited to, e.g., about 0.00001, 0.0001, 0.001, 0.01, 0.02, 0.03, 0.04, 0.05, 0.06, 0.07, 0.08, 0.09, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.25, 1.5, 1.75, 2, 2.5, 3, 4, 5, 6, 7, 8, 9, 10, 15, 20, 30, 40, 50, 60, 70, 80, 90, 100, 200, 300, 400, 500, 600, 700, 800, 900, 1000, 2500, 5000, or 10000 mg/kg/day, or according to the requirements of the particular pharmaceutical composition.

When the pharmaceutical compositions disclosed herein include a combination of a compound of the formulae described herein (e.g., a HPK1 degraders/disruptors) and one or more additional compounds (e.g., one or more additional compounds, drugs, or agents used for the treatment of cancer or any other condition or disease, including conditions or diseases known to be associated with or caused by cancer), both the compound and the additional compound should be present at dosage levels of between about 1 to 100%, and more preferably between about 5 to 95% of the dosage normally administered in a monotherapy regimen. The additional agents can be administered separately, as part of a multiple dose regimen, from the compounds of this invention. Alternatively, those agents can be part of a single dosage form, mixed together with the compounds of this invention in a single composition.

In some aspects, the pharmaceutical compositions disclosed herein can be included in a container, pack, or dispenser together with instructions for administration.

### Methods of Treatment

The methods disclosed herein contemplate administration of an effective amount of a compound or composition to achieve the desired or stated effect. Typically, the compounds or compositions of the invention will be administered from about 1 to about 6 times per day or, alternately or in addition, as a continuous infusion. Such administration can be used as a chronic or acute therapy. The amount of active ingredient that can be combined with the carrier materials to produce a single dosage form will vary depending upon the host treated and the particular mode of administration. A typical preparation will contain from about 5% to about 95% active compound (w/w). Alternatively, such preparations can contain from about 20% to about 80% active compound.

In some aspects, the present disclosure provides methods for using a composition comprising a HPK1 degrader/disruptor, including pharmaceutical compositions (indicated below as ‘X’) disclosed herein in the following methods:

Substance X for use as a medicament in the treatment of one or more diseases or conditions disclosed herein (e.g., cancer, referred to in the following examples as ‘Y’). Use of substance X for the manufacture of a medicament for the treatment of Y; and substance X for use in the treatment of Y.

In some aspects, the methods disclosed include the administration of a therapeutically effective amount of one or more of the compounds or compositions described herein to a subject (e.g., a mammalian subject, e.g., a human subject) who is in need of, or who has been determined to be in need of, such treatment. In some aspects, the methods disclosed include selecting a subject and administering to the subject an effective amount of one or more of the compounds or compositions described herein, and optionally repeating administration as required for the prevention or treatment of cancer.

In some aspects, subject selection can include obtaining a sample from a subject (e.g., a candidate subject) and testing the sample for an indication that the subject is suitable for selection. In some aspects, the subject can be confirmed or identified, e.g. by a health care professional, as having had or having a condition or disease. In some aspects, suitable subjects include, for example, subjects who have or had a condition or disease but that resolved the disease or an aspect thereof, present reduced symptoms of disease (e.g., relative to other subjects (e.g., the majority of subjects) with the same condition or disease), or that survive for extended periods of time with the condition or disease (e.g., relative to other subjects (e.g., the majority of subjects) with the same condition or disease), e.g., in an asymptomatic state (e.g., relative to other subjects (e.g., the majority of subjects) with the same condition or disease). In some aspects, exhibition of a positive immune response towards a condition or disease can be made from patient records, family history, or detecting an indication of a positive immune response. In some aspects, multiple parties can be included in subject selection. For example, a first party can obtain a sample from a candidate subject and a second party can test the sample. In some aspects, subjects can be selected or referred by a medical practitioner (e.g., a general practitioner). In some aspects, subject selection can include obtaining a sample from a selected subject and storing the sample or using the in the methods disclosed herein. Samples can include, e.g., cells or populations of cells.

In some aspects, methods of treatment can include a single administration, multiple administrations, and repeating administration of one or more compounds disclosed herein as required for the prevention or treatment of the disease or condition from which the subject is suffering (e.g., an HPK1-mediated cancer). In some aspects, methods of treatment can include assessing a level of disease in the subject prior to treatment, during treatment, or after treatment. In some aspects, treatment can continue until a decrease in the level of disease in the subject is detected.

The term “subject,” as used herein, refers to any animal. In some instances, the subject is a mammal. In some instances, the term “subject,” as used herein, refers to a human (e.g., a man, a woman, or a child).

The terms “administer,” “administering,” or “administration,” as used herein, refer to implanting, ingesting, injecting, inhaling, or otherwise absorbing a compound or composition, regardless of form. For example, the methods disclosed herein include administration of an effective amount of a compound or composition to achieve the desired or stated effect.

The terms “treat”, “treating,” or “treatment,” as used herein, refer to partially or completely alleviating, inhibiting, ameliorating, or relieving the disease or condition from which the subject is suffering. This means any manner in which one or more of the symptoms of a disease or disorder (e.g., cancer) are ameliorated or otherwise beneficially altered. As used herein, amelioration of the symptoms of a particular disorder (e.g., cancer) refers to any lessening, whether permanent or temporary, lasting or transient that can be attributed to or associated with treatment by the compositions and methods of the present invention. In some aspects, treatment can promote or result in, for example, a decrease in the number of tumor cells (e.g., in a subject) relative to the number of tumor cells prior to treatment; a decrease in the viability (e.g., the average/mean viability) of tumor cells (e.g., in a subject) relative to the viability of tumor cells prior to treatment; a decrease in the rate of growth of tumor cells; a decrease in the rate of local or distant tumor metastasis; or reductions in one or more symptoms associated with one or more tumors in a subject relative to the subject's symptoms prior to treatment.

As used herein, the term “treating cancer” means causing a partial or complete decrease in the rate of growth of a tumor, and/or in the size of the tumor and/or in the rate of local or distant tumor metastasis, and/or the overall tumor burden in a subject, and/or any decrease in tumor survival, in the presence of a degrader/disruptor (e.g., an HPK1 degrader/disruptor) described herein.

The terms “prevent,” “preventing,” and “prevention,” as used herein, shall refer to a decrease in the occurrence of a disease or decrease in the risk of acquiring a disease or its associated symptoms in a subject. The prevention may be complete, e.g., the total absence of disease or pathological cells in a subject. The prevention may also be partial, such that the occurrence of the disease or pathological cells in a subject is less than, occurs later than, or develops more slowly than that which would have occurred without the present invention.

Exemplary type of cancers that could be prevented, or therapeutically treated by manipulation of HPK1 level by degraders/disruptors should include all solid and liquid cancers, including, but not limited to, cancers of the breast, respiratory tract, brain, reproductive organs, digestive tract, urinary tract, eye, liver, skin, head and neck, thyroid, parathyroid and their distant metastases. Examples of liquid cancers include lymphomas, sarcomas, and leukaemias. Listed below are the type of cancers that immunotherapy using HPK1 degraders/disruptors should be able to prevent or treat.

Examples of breast cancers include, but are not limited to, triple negative breast cancer, invasive ductal carcinoma, invasive lobular carcinoma, ductal carcinoma in situ, and lobular carcinoma in situ.

Examples of cancers of the respiratory tract include, but are not limited to, small-cell and non-small-cell lung carcinoma, as well as bronchial adenoma and pleuropulmonary blastoma.

Examples of brain cancers include, but are not limited to, brain stem and hypophtalmic glioma, cerebellar and cerebral astrocytoma, glioblastoma, medulloblastoma, ependymoma, as well as neuroectodermal and pineal tumor.

Tumors of the male reproductive organs include, but are not limited to, prostate and testicular cancer.

Tumors of the female reproductive organs include, but are not limited to, endometrial, cervical, ovarian, vaginal, and vulvar cancer, as well as sarcoma of the uterus.

Examples of ovarian cancer include, but are not limited to, serous tumor, endometrioid tumor, mucinous cystadenocarcinoma, granulosa cell tumor, Sertoli-Leydig cell tumor and arrhenoblastoma.

Examples of cervical cancer include, but are not limited to, squamous cell carcinoma, adenocarcinoma, adenosquamous carcinoma, small cell carcinoma, neuroendocrine tumor, glassy cell carcinoma and villoglandular adenocarcinoma. Tumors of the digestive tract include, but are not limited to, anal, colon, colorectal, esophageal, gallbladder, gastric, pancreatic, rectal, small-intestine, and salivary gland cancers.

Examples of esophageal cancer include, but are not limited to, esophageal cell carcinomas and adenocarcinomas, as well as squamous cell carcinomas, leiomyosarcoma, malignant melanoma, rhabdomyosarcoma and lymphoma.

Examples of gastric cancer include, but are not limited to, intestinal type and diffuse type gastric adenocarcinoma.

Examples of pancreatic cancer include, but are not limited to, ductal adenocarcinoma, adenosquamous carcinomas and pancreatic endocrine tumors.

Example of tumors of the urinary tract include, but are not limited to, bladder, penile, kidney, renal pelvis, ureter, urethral and human papillary renal cancers.

Examples of kidney cancer include, but are not limited to, renal cell carcinoma, urothelial cell carcinoma, juxtaglomerular cell tumor (reninoma), angiomyolipoma, renal oncocytoma, Bellini duct carcinoma, clear-cell sarcoma of the kidney, mesoblastic nephroma and Wilms' tumor.

Examples of bladder cancer include, but are not limited to, transitional cell carcinoma, squamous cell carcinoma, adenocarcinoma, sarcoma and small cell carcinoma. Eye cancers include, but are not limited to, intraocular melanoma and retinoblastoma.

Examples of liver cancers include, but are not limited to, hepatocellular carcinoma (liver cell carcinomas with or without fibrolamellar variant), cholangiocarcinoma (intrahepatic bile duct carcinoma), and mixed hepatocellular cholangiocarcinoma.

Example of skin cancers include, but are not limited to, squamous cell carcinoma, Kaposi's sarcoma, malignant melanoma, Merkel cell skin cancer, and non-melanoma skin cancer.

Example of head-and-neck cancers include, but are not limited to, squamous cell cancer of the head and neck, laryngeal, hypopharyngeal, nasopharyngeal, oropharyngeal cancer, salivary gland cancer, lip and oral cavity cancer and squamous cell.

Example of lymphomas include, but are not limited to, AIDS-related lymphoma, non-Hodgkin's lymphoma, cutaneous T-cell lymphoma, Burkitt lymphoma, Hodgkin's disease, and lymphoma of the central nervous system.

Example of sarcomas include, but are not limited to, sarcoma of the soft tissue, osteosarcoma, malignant fibrous histiocytoma, lymphosarcoma, and rhabdomyosarcoma.

Example of leukemias include, but are not limited to, acute myeloid leukemia, acute lymphoblastic leukemia, chronic lymphocytic leukemia, chronic myelogenous leukemia, and hairy cell leukemia.

The HPK1 degraders/disruptors should be able to treat the above cancer type as stand alone agent or used as agent in combination with existing standard of treatment therapy and other FDA-approved cancer therapy.

Therapeutic uses of HPK1 include diseases and therapies that are amenable to treatment by stimulation/augmentation of immune response, including the prolongation of immune responses during vaccination for immunizable diseases. Also, because HPK1 is expressed at high level in two other anatomical locations—brain and testes—the HPK1 degraers/disruptors should be able to treat or prevent diseases related to brain and testes that were caused by HPK1 or could be treated by HPK1 degraders/disruptors. These potential diseases include, but is not limited to, Alzheimer's disease, age-related dementia and infertility, regardless whether these possible diseases were caused by HPK1 or by other ethological causes.

As used herein, the term “preventing a disease” (e.g., preventing cancer) in a subject means for example, to stop the development of one or more symptoms of a disease in a subject before they occur or are detectable, e.g., by the patient or the patient's doctor. A blood test that measure the level of HPK1 in each of the immune cell sub-types, which could be achieved by intracellular staining by anti-HPK1 antibody and analyze by clinical FACS analysis. Such detection method could identify immune cell type possess aberrant level of HPK1 and may signify that such patient might be a good candidate for HPK1 degraders/disruptors-based therapy. This detection of aberrant expression level of HPK1 may be an early warning biomarker that may indicate which patient may respond well to their disease conditions if HPK1 degraders/disruptors were to used as stand alone or as part of combination therapy. Preferably, the disease (e.g., cancer) does not develop at all, i.e., no symptoms of the disease are detectable. However, it can also mean delaying or slowing of the development of one or more symptoms of the disease. Alternatively, or in addition, it can mean decreasing the severity of one or more subsequently developed symptoms.

Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

An effective amount can be administered in one or more administrations, applications or dosages. A therapeutically effective amount of a therapeutic compound (i.e., an effective dosage) depends on the therapeutic compounds selected. Moreover, treatment of a subject with a therapeutically effective amount of the compounds or compositions described herein can include a single treatment or a series of treatments. For example, effective amounts can be administered at least once. The compositions can be administered one from one or more times per day to one or more times per week; including once every other day. The skilled artisan will appreciate that certain factors can influence the dosage and timing required to effectively treat a subject, including but not limited to the severity of the disease or disorder, previous treatments, the general health or age of the subject, and other diseases present.

Following administration, the subject can be evaluated to detect, assess, or determine their level of disease. In some instances, treatment can continue until a change (e.g., reduction) in the level of disease in the subject is detected. Upon improvement of a patient's condition (e.g., a change (e.g., decrease) in the level of disease in the subject), a maintenance dose of a compound, or composition disclosed herein can be administered, if necessary. Subsequently, the dosage or frequency of administration, or both, can be reduced, e.g., as a function of the symptoms, to a level at which the improved condition is retained. Patients may, however, require intermittent treatment on a long-term basis upon any recurrence of disease symptoms.

The HPK1 degraders/disruptors disclosed herein include pure enantiomers, mixtures of enantiomers, pure diastereoisomers, mixtures of diastereoisomers, diastereoisomeric racemates, mixtures of diastereoisomeric racemates and the meso-form and pharmaceutically acceptable salts, solvent complexes, morphological forms, or deuterated and fluoro derivatives thereof.

### EXAMPLES

The Following examples describe the synthesis of exemplary HPK1 degrader/disrupter compounds according to the present invention.

### Example 1

### Synthesis of MS-5-006

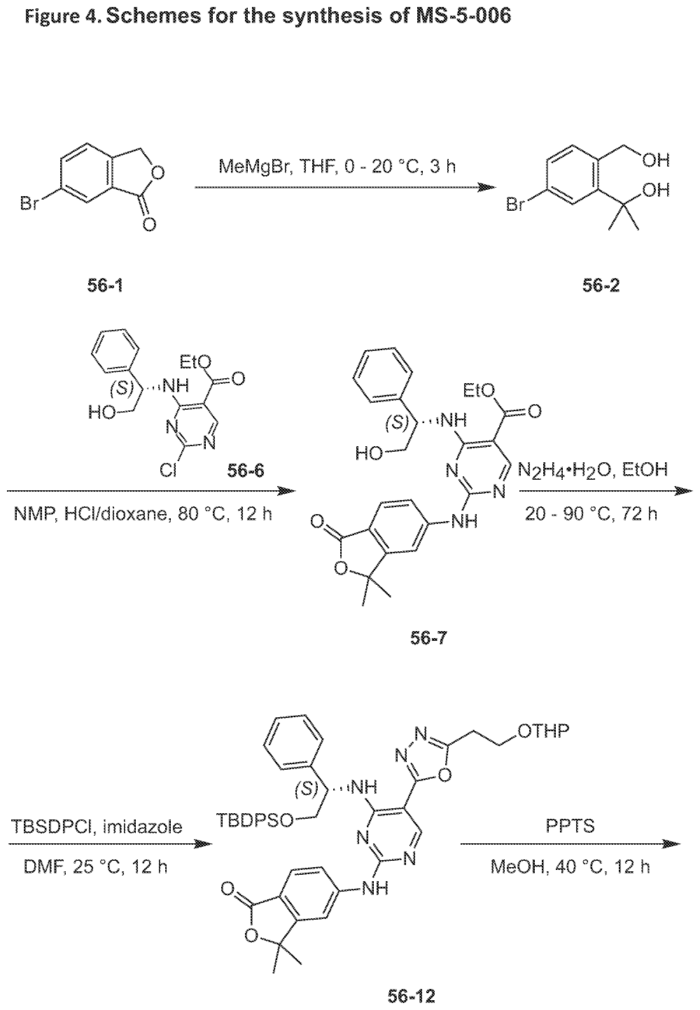

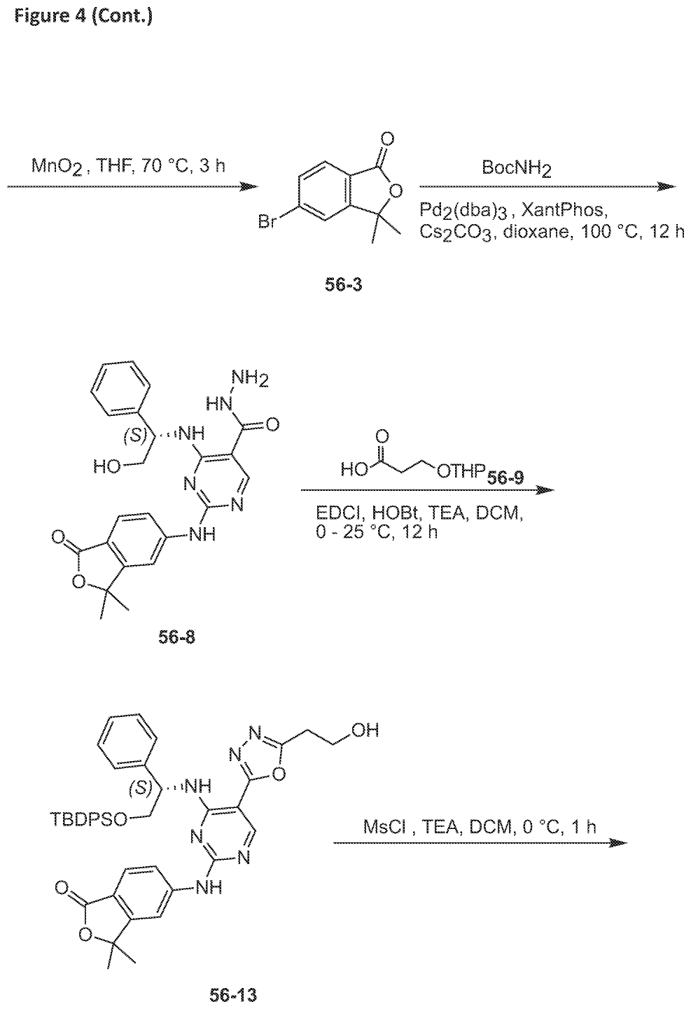

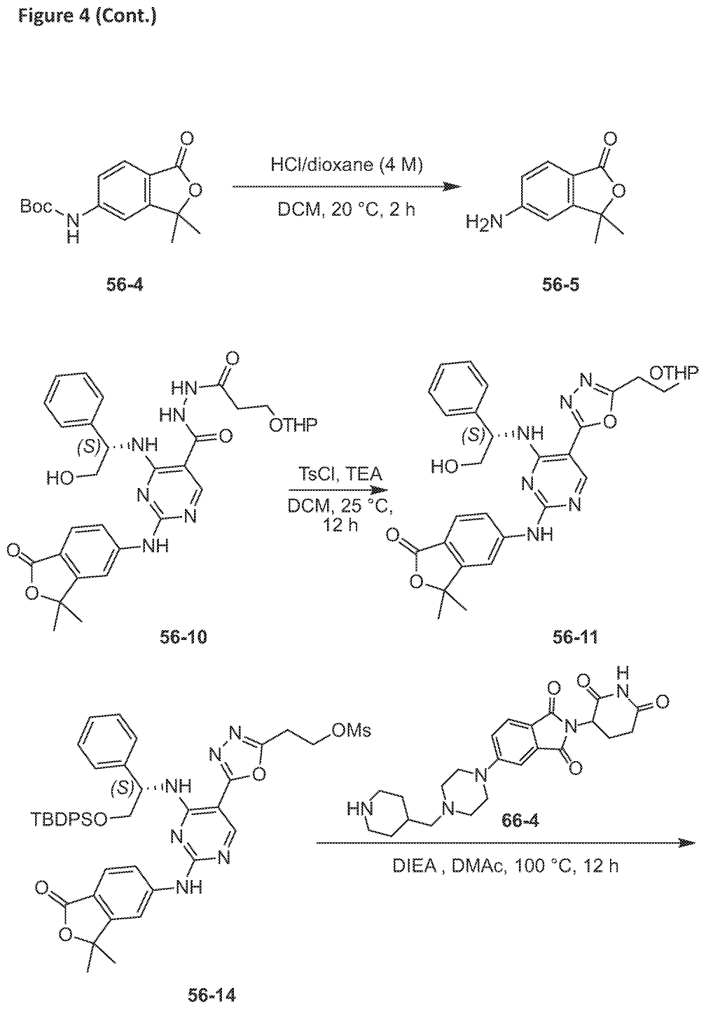

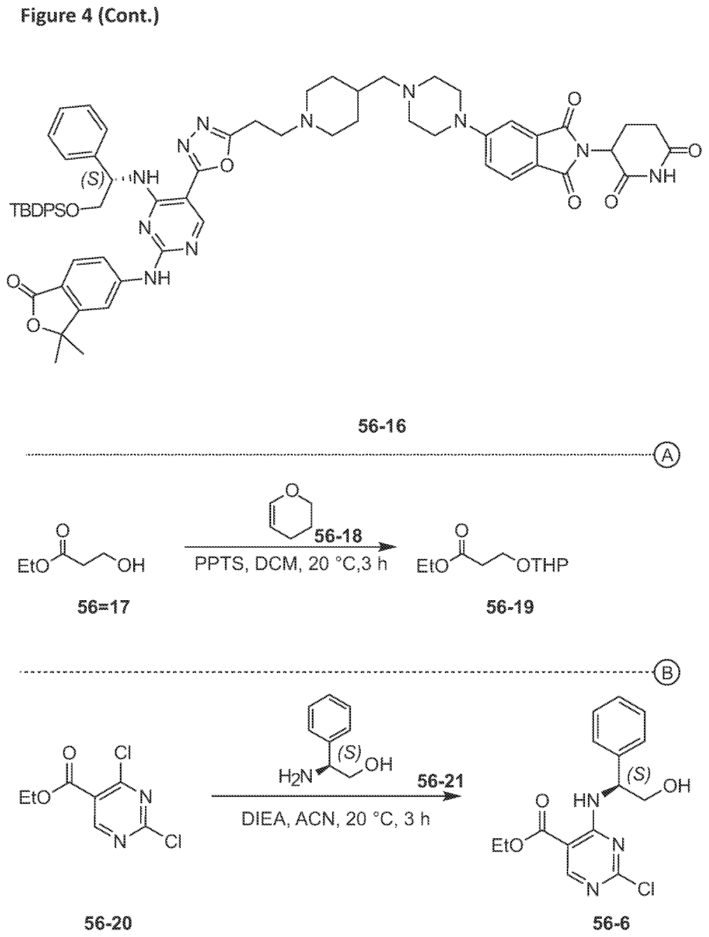

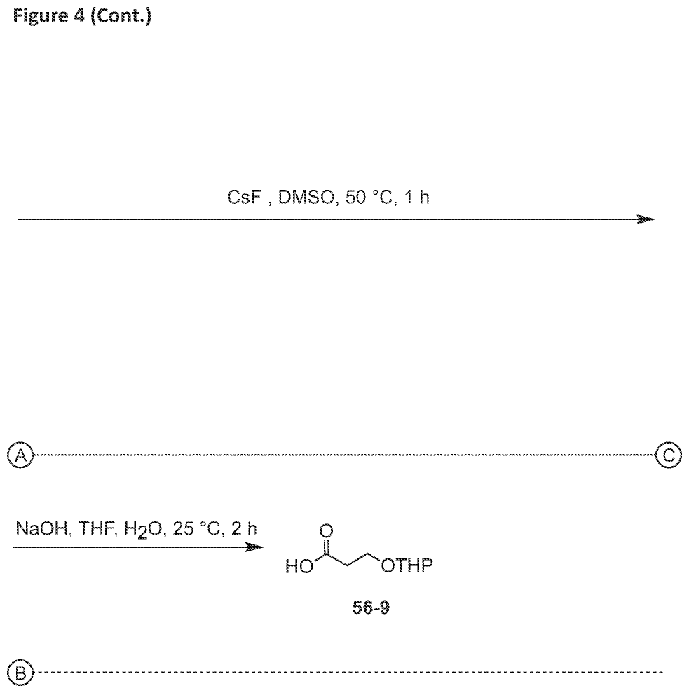

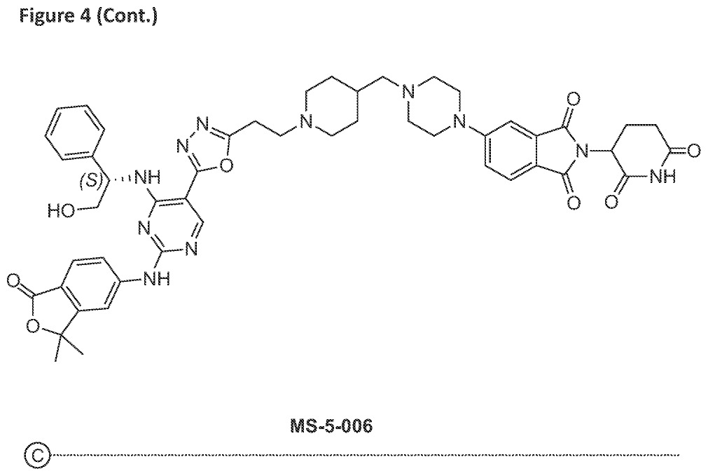

The reaction scheme for the synthesis of MS-5-006 is shown in FIG. 4.

The procedure for preparing 66-4 is described in the synthesis of MS-6-006.

Procedure for preparation of compound 56-2. To a solution of compound 56-1 (25 g, 117.36 mmol, 1 eq) in THE (250 mL) was added MeMgBr (3 M, 117.36 mL, 3 eq) at 0° C. The mixture was stirred at 20° C. for 3 h. The reaction was quenched by the addition of aq. NH₄Cl (500 mL). The aqueous phase was extracted with DCM (250 mL×3). The combined organic extracts were washed with brine (50 mL), dried, filtered and concentrated. The residue was purified by flash silica gel chromatography (SiO₂, PE/EA=100/1 to 1/1) to give compound 56-2 (19.6 g, 79.96 mmol, 68.14% yield) as a white solid. ¹H NMR (400 MHz, CDCl₃): δ=7.43 (d, J=2.0 Hz, 1H), 7.36 (dd, J=8.4, 2.0 Hz, 1H), 7.17 (d, J=8.0 Hz, 1H), 4.76 (s, 2H), 3.69-3.12 (m, 2H), 1.66 (s, 6H).

Procedure for preparation of compound 56-3. To a solution of compound 56-2 (19 g, 77.52 mmol, 1 eq) in THE (130 mL) was added MnO₂ (67.39 g, 775.15 mmol, 10 eq). The mixture was stirred at 70° C. for 3 h. The reaction mixture was filtered and the filtrate was concentrated under the reduced pressure to give compound 56-3 (18.55 g, 76.95 mmol, 99.26% yield) as a yellow solid. LCMS: t_{R}=434 min, MS (ESI+) m/z=240.7 [M+1]⁺. ¹H NMR (400 MHz, CDCl₃): δ=7.80-7.69 (m, 1H), 7.69-7.61 (m, 1H), 7.57 (s, 1H), 1.67 (s, 6H).

Procedure for preparation of compound 56-4. A mixture of compound 56-3 (18 g, 74.66 mmol, 1 eq), BocNH₂ (10.50 g, 89.60 mmol, 1.2 eq), Pd₂(dba)₃ (3.42 g, 3.73 mmol, 0.05 eq), Cs₂CO₃ (48.65 g, 149.33 mmol, 2 eq) and Xantphos (4.32 g, 7.47 mmol, 0.1 eq) in dioxane (180 mL) was degassed and purged with N₂ for 3 times, and then the mixture was stirred at 100° C. for 12 h under N₂ atmosphere. The reaction mixture were washed with brine (250 mL), and extracted with EtOAc (250 mL×3). The combined organic layers were dried over anhydrous Na₂SO₄, filtered and concentrated under reduced pressure to give a residue. The residue was purified by flash silica gel chromatography (SiO₂, DCM/MeOH=100/1 to 10/1) to give compound 56-4 (21.5 g) as a yellow solid. LCMS: t_{R}=0.450 min, MS (ESI+) m/z=278.1[M+1]⁺. ¹H NMR (400 MHz, DMSO-d₆): δ=9.97 (s, 1H), 7.88 (d, J=1.2 Hz, 1H), 7.67 (d, J=8.4 Hz, 1H), 7.42 (dd, J=8.4, 2.0 Hz, 1H), 1.57 (s, 6H), 1.49 (s, 9H).

Procedure for preparation of compound 56-5. To a solution of compound 56-4 (21.5 g, 77.53 mmol, 1 eq) in DCM (120 mL) was added HCl/dioxane (4 M, 58.15 mL, 3 eq). The mixture was stirred at 20° C. for 2 h. The reaction mixture was filtered and the filtrate was concentrated under the reduced pressure to give compound 56-5 (10.93 g, 51.16 mmol, 65.98% yield, HCl) as a yellow solid. ¹H NMR (400 MHz, DMSO-d₆): δ=7.40 (d, J=8.4 Hz, 1H), 6.67 (dd, J=8.0, 1.6 Hz, 1H), 6.58 (d, J=2.0 Hz, 1H), 1.51 (s, 6H).

Procedure for preparation of compound 56-6. To a solution of compound 56-20 (40 g, 180.96 mmol, 1 eq) and compound 56-21 (26.07 g, 190.01 mmol, 1.05 eq) in ACN (600 mL) was added DIEA (46.78 g, 361.92 mmol, 63.04 mL, 2 eq). The mixture was stirred at 20° C. for 3 h. The reaction mixture was quenched by addition water (800 mL), and the mixture was filtered to give compound 56-6 (54.2 g, crude) as a yellow solid. LCMS: t_{R}=0.447 min, MS (ESI+) m/z=322.7 [M+1]⁺. ¹H NMR (400 MHz, CDCl₃): δ=9.15 (d, J=7.6 Hz, 1H), 8.69 (s, 1H), 7.39 (d, J=4.4 Hz, 4H), 7.36-7.29 (m, 1H), 5.48 (m, 1H), 4.39 (m, 2H), 4.07-3.91 (m, 2H), 1.41 (t, J=7.2 Hz, 3H).

Procedure for preparation of compound 56-7. To a solution of compound 56-5 (10.9 g, 51.02 mmol, 1 eq, HCl) and compound 56-6 (19.79 g, 61.51 mmol, 1.21 eq) in NMP (70 mL) was added HCl/dioxane (4 M, 23.07 mL, 1.81 eq). The mixture was stirred at 80° C. for 12 h. The reaction was cooled to room temperature, quenched by addition of triethylamine (25 mL), and diluted with EtOAc (150 mL) and ethanol (7.5 mL). The mixture was washed with brine (150 mL×3), dried over Na₂SO₄, filtered and concentrated under reduced pressure to give compound 56-7 (24.95 g, crude) as a yellow solid. LCMS: t_{R}=0.445 min, MS (ESI+) m/z=463.1 [M+1]⁺. ¹H NMR (400 MHz, DMSO-d₆): δ=10.30 (s, 1H), 9.00 (d, J=7.6 Hz, 1H), 8.68 (s, 1H), 8.01-7.84 (m, 1H), 7.76-7.65 (m, 1H), 7.64-7.56 (m, 1H), 7.44-7.31 (m, 4H), 7.30-7.22 (m, 1H), 5.39-5.26 (m, 1H), 5.19 (t, J=4.8 Hz, 1H), 4.37-4.22 (m, 2H), 3.93-3.84 (m, 1H), 3.80 (m, 1H), 1.57 (s, 3H), 1.51 (s, 3H), 1.34 (t, J=6.8 Hz, 3H).

Procedure for preparation of compound 56-19. To a solution of compound 56-17 (10 g, 84.65 mmol, 1 eq) in DCM (100 mL) was added PPTS (1.06 g, 4.23 mmol, 0.05 eq) and compound 56-18 (9.97 g, 118.51 mmol, 10.84 mL, 1.4 eq). The mixture was stirred at 20° C. for 3 h. The reaction mixture was partitioned between DCM (150 mL×2) and water (150 mL). After quenching the reaction, the reaction mixture was poured into separatory funnel and separated. The residue was purified by flash silica gel chromatography (SiO₂, PE/EA=100/1 to 4/1) to give compound 56-19 (10.5 g, 51.92 mmol, 61.33% yield) as a colorless oil. ¹H NMR (400 MHz, CDCl₃): δ=4.63 (t, J=2.8 Hz, 1H), 4.21-4.09 (m, 2H), 4.00 (td, J=10.0, 6.4 Hz, 1H), 3.93-3.78 (m, 1H), 3.69 (td, J=10.0, 6.4 Hz, 1H), 3.59-3.44 (m, 1H), 2.60 (t, J=6.4 Hz, 2H), 1.87-1.75 (m, 1H), 1.74-1.64 (m, 1H), 1.62-1.45 (m, 4H), 1.31-1.22 (m, 3H).

Procedure for preparation of compound 56-9. To a solution of compound 56-19 (10.5 g, 51.92 mmol, 1 eq) in THE (100 mL) was added NaOH (2 M, 51.92 mL, 2 eq). The mixture was stirred at 25° C. for 2 h. The reaction mixture were washed with citric acid (5%, 100 mL), and extracted with EtOAc (50 mL×3). The combined organic layers were dried over anhydrous Na₂SO₄, filtered and concentrated under reduced pressure to give compound 56-9 (5.71 g, 32.78 mmol, 63.14% yield) as a colourless oil. ¹H NMR (400 MHz, CDCl₃): δ=4.65 (t, J=3.2 Hz, 1H), 4.01 (td, J=10.0, 6.0 Hz, 1H), 3.93-3.81 (m, 1H), 3.71 (td, J=10.0, 6.4 Hz, 1H), 3.62-3.41 (m, 1H), 2.66 (t, J=6.4 Hz, 2H), 1.86-1.75 (m, 1H), 1.75-1.66 (m, 1H), 1.66-1.45 (m, 4H).

Procedure for preparation of compound 56-8. To a solution of compound 56-7 (5 g, 10.81 mmol, 1 eq) in EtOH (250 mL) was added NH₂NH₂·H₂O (17.73 g, 354.17 mmol, 17.21 mL, 32.76 eq) slowly at 20° C. The mixture was stirred at 90° C. for 72 h. The reaction mixture was concentrated to afford the crude product which was purified by prep-HPLC (HCl condition) and lyophilized to give compound 56-8 (5.25 g, crude) as a yellow solid. LCMS: t_{R}=0.340 min, MS (ESI+) m/z=449.0 [M+1]⁺. ¹H NMR (400 MHz, DMSO-d₆): δ=10.06 (s, 2H), 9.62 (d, J=8.0 Hz, 1H), 8.57 (s, 1H), 7.96 (d, J=1.2 Hz, 1H), 7.74-7.65 (m, 1H), 7.64-7.56 (m, 1H), 7.41-7.30 (m, 4H), 7.29-7.19 (m, 1H), 5.80-4.48 (m, 4H), 3.89-3.79 (m, 1H), 3.78-3.66 (m, 1H), 1.54 (d, J=18.8 Hz, 6H).

Procedure for preparation of compound 56-10. To a solution of compound 56-9 (1.17 g, 6.69 mmol, 1 eq) in DCM (45 mL) was added EDCI (1.28 g, 6.69 mmol, 1 eq), HOBt (903.89 mg, 6.69 mmol, 1 eq) and TEA (744.58 mg, 7.36 mmol, 1.02 mL, 1.1 eq) at 0° C. for 30 min. Then compound 56-8 (3 g, 6.69 mmol, 1 eq) was added. The reaction mixture was partitioned between DCM (50 mL×2) and H₂O (50 mL). After quenching the reaction, the reaction mixture was poured into separatory funnel and separated. The residue was purified by prep-HPLC (22%-53% ACN in water (0.225% FA), 20 min) and lyophilized to give compound 56-10 (2.43 g, 3.45 mmol, 51.62% yield, 86% purity) as a white solid. LCMS: t_{R}=0.392 min, MS (ESI+) m/z=605.2 [M+1]⁺. ¹H NMR (400 MHz, DMSO-d₆): δ=10.25 (s, 1H), 10.14 (s, 1H), 9.85 (s, 1H), 9.54 (d, J=7.6 Hz, 1H), 8.68 (s, 1H), 7.97 (s, 1H), 7.73-7.66 (m, 1H), 7.65-7.59 (m, 1H), 7.36 (d, J=4.0 Hz, 4H), 7.25 (m, 1H), 5.30 (m, 1H), 5.14 (t, J=4.8 Hz, 1H), 4.62-4.55 (m, 1H), 3.96-3.53 (m, 6H), 3.49-3.39 (m, 1H), 2.48-2.45 (m, 1H), 1.81-1.62 (m, 2H), 1.58 (s, 3H), 1.53 (s, 3H), 1.50-1.41 (m, 4H).

Procedure for preparation of compound 56-11. To a solution of compound 56-10 (2.4 g, 3.97 mmol, 1 eq) in DCM (25 mL) was added TsCl (832.39 mg, 4.37 mmol, 1.1 eq) and TEA (602.46 mg, 5.95 mmol, 828.70 uL, 1.5 eq). The mixture was stirred at 25° C. for 12 h. Acidify the reaction mixture by adding, with shaking, 50 mL of NaHCO₃ until pH around 8, and then extracted with DCM (30 mL×2). The combined organic layers were dried over anhydrous Na₂SO₄, filtered and concentrated to give a residue. The residue was purified by flash silica gel chromatography (SiO₂, DCM/MeOH=100/1 to 10/1) to give compound 56-11 (2.51 g, crude) as a yellow solid. LCMS: t_{R}=0.464 min, MS (ESI+) m/z=587.1 [M+1]⁺. ¹H NMR (400 MHz, DMSO-d₆): δ=10.30 (s, 1H), 8.87 (d, J=8.0 Hz, 1H), 8.65 (s, 1H), 7.97 (s, 1H), 7.79-7.67 (m, 1H), 7.67-7.60 (m, 1H), 7.45-7.33 (m, 4H), 7.30-7.23 (m, 1H), 5.52-5.35 (m, 1H), 5.24 (t, J=4.8 Hz, 1H), 4.73-4.62 (m, 1H), 4.10-4.01 (m, 1H), 3.97-3.89 (m, 1H), 3.89-3.79 (m, 2H), 3.74-3.64 (m, 1H), 3.49-3.40 (m, 1H), 3.23 (t, J=6.4 Hz, 2H), 1.72-1.61 (m, 2H), 1.58 (s, 3H), 1.53 (s, 3H), 1.50-1.37 (m, 4H).

Procedure for preparation of compound 56-12. To a solution of compound 56-11 ((2.4 g, 4.09 mmol, 1 eq) in DMF (24 mL) was added TBDPSCl (3.37 g, 12.27 mmol, 3.15 mL, 3 eq) and imidazole (1.11 g, 16.36 mmol, 4 eq). The mixture was stirred at 25° C. for 12 h. The reaction mixture was partitioned between EtOAc (30 mL×2) and H₂O (30 mL). After quenching the reaction, the reaction mixture was poured into separatory funnel and separated. The residue was purified by flash silica gel chromatography (SiO₂, DCM/MeOH=100/1 to 10/1) to give compound 56-12 (2.69 g, 3.26 mmol, 79.70% yield) as a colourless solid. LCMS: t_{R}=0.664 min, MS (ESI+) m/z=825.1 [M+1]⁺. ¹H NMR (400 MHz, CDCl₃): δ=9.31 (d, J=7.6 Hz, 1H), 8.63 (s, 1H), 7.72 (d, J=8.4 Hz, 1H), 7.61 (dd, J=8.0, 1.2 Hz, 3H), 7.55 (dd, J=8.4, 1.6 Hz, 1H), 7.45-7.35 (m, 8H), 7.35-7.25 (m, 6H), 5.57-5.45 (m, 1H), 4.73 (s, 1H), 4.23 (m, 1H), 4.12-4.05 (m, 1H), 4.03-3.91 (m, 2H), 3.91-3.81 (m, 1H), 3.61-3.51 (m, 1H), 3.30 (t, J=6.4 Hz, 2H), 1.85-1.67 (m, 3H), 1.63 (d, J=2.0 Hz, 6H), 1.60-1.47 (m, 3H), 1.02 (s, 9H).

Procedure for preparation of compound 56-13. To a solution of compound 56-12 (2.6 g, 3.15 mmol, 1 eq) in MeOH (26 mL) was added PPTS (79.19 mg, 315.14 μmol, 0.1 eq). The mixture was stirred at 40° C. for 12 h. The reaction mixture was concentrated under the reduced pressure to give a residue. The residue was purified by flash silicagel chromatography (SiO₂, DCM/MeOH=100/1 to 10/1) to give compound 56-13 (1.91 g, 2.58 mmol, 81.80% yield) as a colourless solid. LCMS: t_{R}=0.597 min, MS (ESI+) m/z=741.2 [M+1]⁺. ¹H NMR (400 MHz, CDCl₃): δ=9.30 (d, J=8.0 Hz, 1H), 8.61 (s, 1H), 7.70 (d, J=8.4 Hz, 1H), 7.64-7.57 (m, 3H), 7.56-7.50 (m, 2H), 7.43-7.26 (m, 13H), 5.58-5.40 (m, 1H), 4.18 (t, J=5.6 Hz, 2H), 4.13-4.05 (m, 1H), 3.98 (dd, J=10.0, 4.8 Hz, 1H), 3.21 (t, J=5.6 Hz, 2H), 2.76-2.49 (m, 1H), 1.62 (s, 6H), 1.01 (s, 9H).

Procedure for preparation of compound 56-14. To a solution of compound 56-13 (1 g, 1.35 mmol, 1 eq) in DCM (20 mL) was added TEA (273.14 mg, 2.70 mmol, 375.71 uL, 2 eq) and MsCl (309.21 mg, 2.70 mmol, 208.93 uL, 2 eq) at 0° C. The mixture was stirred at 0° C. for 1 h under N₂ atmosphere. The reaction mixture was quenched by H₂O (30 mL) and extracted with DCM (20 mL×2), the organic layer was concentrated under reduced pressure to give compound 56-14 (1.83 g, crude) as a yellow oil. LCMS: t_{R}=0.604 min, MS (ESI+) m/z=819.1 [M+1]⁺.

Procedure for preparation of compound 56-16. To a solution of compound 56-14 (516.22 mg, 630.30 μmol, 1 eq) and compound 66-4 (300 mg, 630.30 μmol, 1 eq, HCl) in DMAc (5 mL) was added DIEA (325.84 mg, 2.52 mmol, 439.13 uL, 4 eq). The mixture was stirred at 100° C. for 12 h. The residue was purified by prep-HPLC (31%-61% ACN in water (0.225% FA), 10 min) and lyophilized to give compound 56-16 (200 mg, 172.06 μmol, 27.30% yield) as a yellow solid. LCMS: t_{R}=0.479 min, MS (ESI+) m/z=1162.8 [M+1]⁺. ¹H NMR (400 MHz, DMSO-d₆): δ=11.07 (s, 1H), 10.29 (s, 1H), 9.23 (d, J=8.0 Hz, 1H), 8.70 (s, 1H), 7.88 (s, 1H), 7.76-7.72 (m, 1H), 7.68 (d, J=8.4 Hz, 1H), 7.61 (d, J=8.4 Hz, 1H), 7.54 (d, J=6.8 Hz, 2H), 7.43-7.23 (m, 15H), 5.63-5.53 (m, 1H), 5.07 (m, 1H), 4.15 (dd, J=10.0, 3.2 Hz, 1H), 3.99 (dd, J=11.6, 4.4 Hz, 1H), 3.56 (m, 2H), 3.42 (d, J=4.8 Hz, 12H), 2.56 (s, 6H), 2.28-2.11 (m, 3H), 2.05-2.00 (m, 1H), 1.82 (d, J=10.8 Hz, 3H), 1.52 (d, J=6.0 Hz, 6H), 0.93 (s, 9H).

Procedure for preparation of MS-5-006. To a solution of compound 56-16 (150 mg, 129.04 μmol, 1 eq) in DMSO (1.5 mL) was added CsF (196.02 mg, 1.29 mmol, 47.58 uL, 10 eq). The mixture was stirred at 50° C. for 1 h. The residue was purified by prep-HPLC (20%-25% ACN in water (0.225% FA), 10 min) and lyophilized to give MS-5-006 (26.74 mg, 27.48 μmol, 21.30% yield, 99.7% purity, FA) as a yellow solid. LCMS: t_{R}=1.134 min, MS (ESI+) m/z=924.6 [M+1]⁺. ¹H NMR (400 MHz, DMSO-d₆): δ=11.08 (s, 1H), 10.30 (s, 1H), 8.88 (d, J=8.0 Hz, 1H), 8.65 (s, 1H), 8.14 (s, 1H), 7.97 (s, 1H), 7.81-7.55 (m, 3H), 7.51-7.31 (m, 5H), 7.30-7.15 (m, 2H), 5.53-5.37 (m, 1H), 5.34-5.15 (m, 1H), 5.07 (dd, J=12.8, 5.2 Hz, 1H), 3.94-3.88 (m, 1H), 3.87-3.81 (m, 1H), 3.13 (t, J=7.2 Hz, 4H), 3.01-2.74 (m, 6H), 2.63-2.52 (m, 2H), 2.46 (s, 4H), 2.16 (d, J=6.8 Hz, 2H), 2.10-1.91 (m, 3H), 1.70 (d, J=11.6 Hz, 2H), 1.55 (d, J=20.0 Hz, 7H), 1.09 (q, J=10.8 Hz, 2H).

### Example 2

### Synthesis of MS-5-007

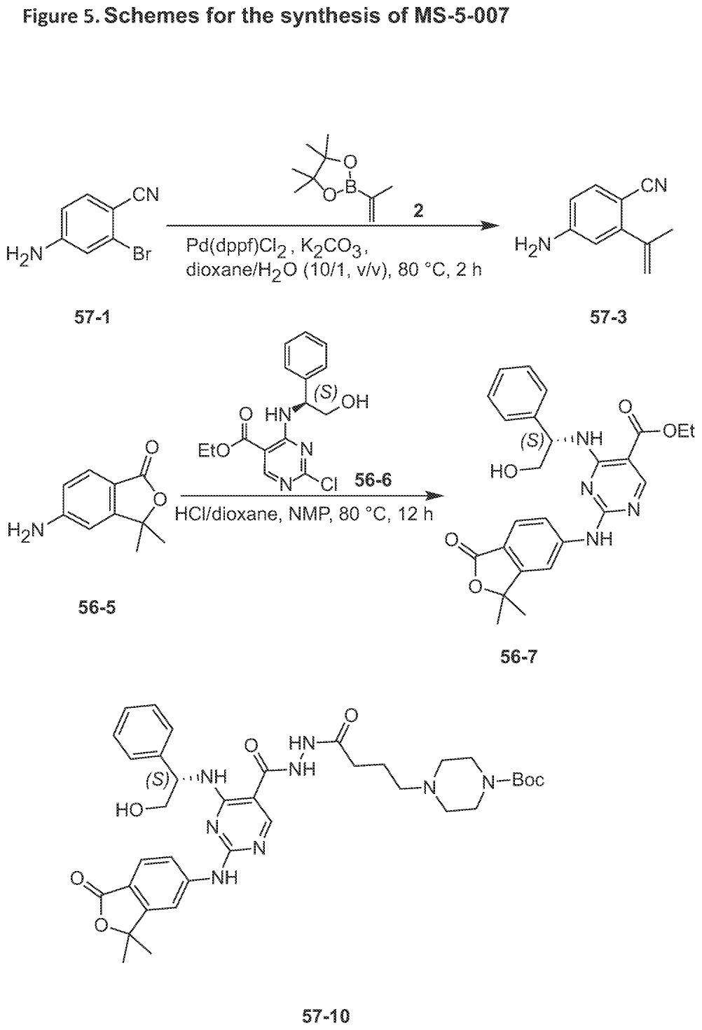

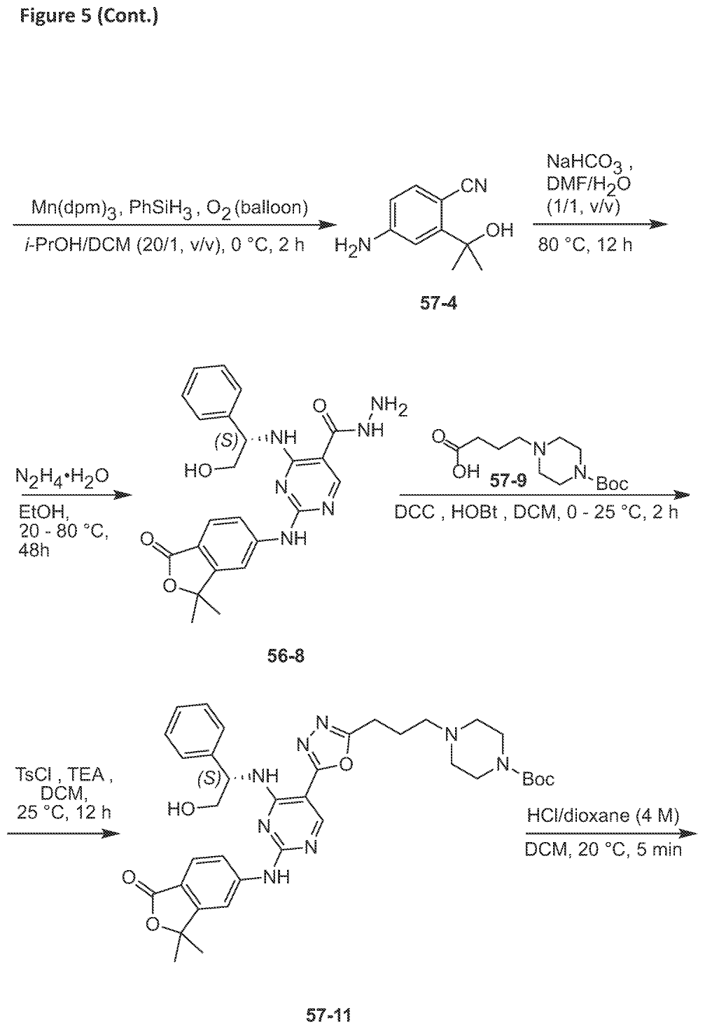

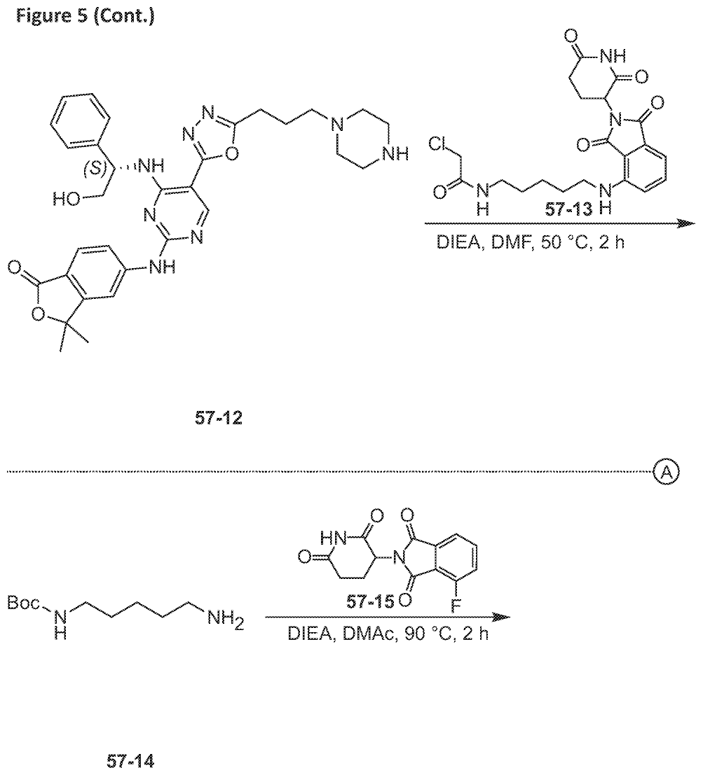

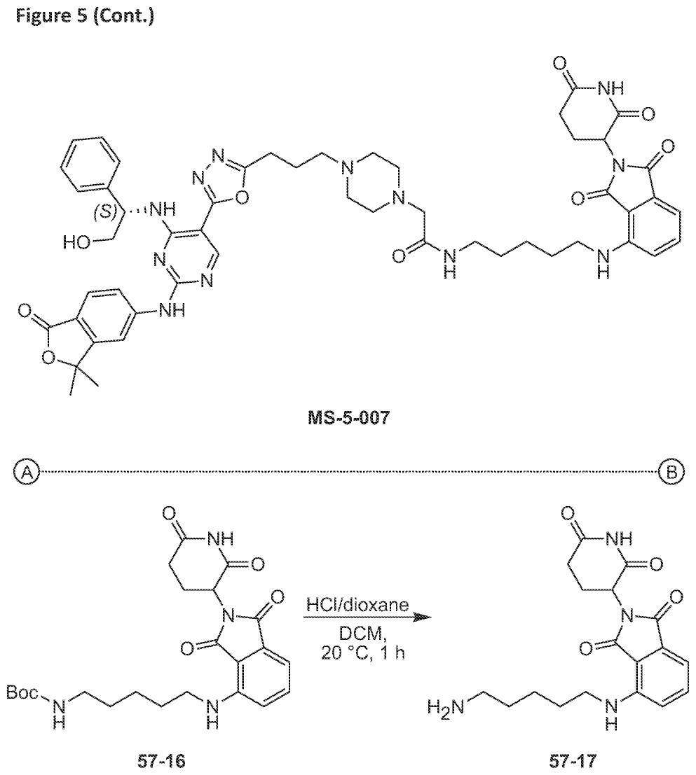

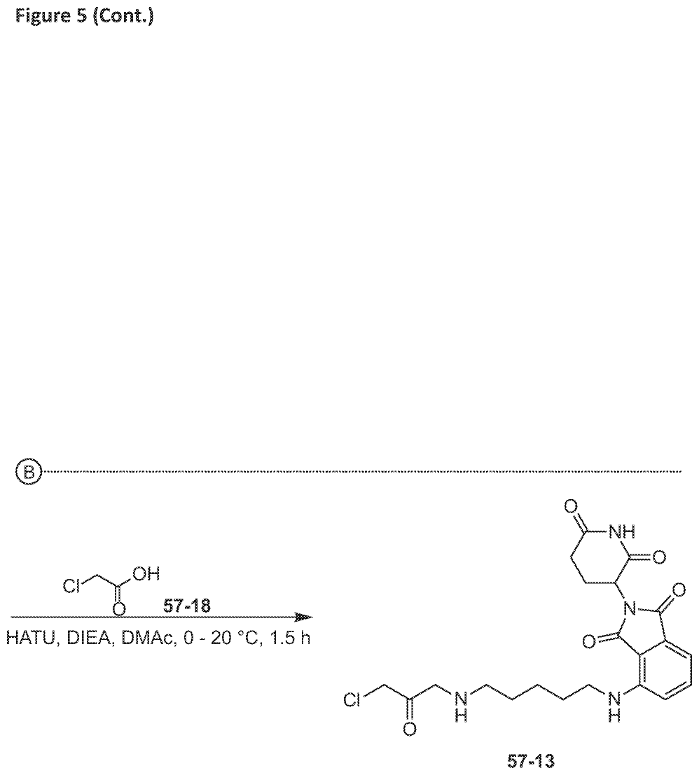

The reaction scheme for the synthesis of MS-5-007 is shown in FIG. 5.

Procedure for preparation of compound 57-3. A flask was purged with nitrogen and charged with a mixture of compound 57-1 (5 g, 25.38 mmol, 1 eq), compound 57-2 (5.12 g, 30.45 mmol, 1.2 eq), dioxane (80 mL) and H₂O (8 mL). To the solution was added K₂CO₃ (7.72 g, 55.83 mmol, 2.2 eq) and Pd(dppf)Cl₂ (1.86 g, 2.54 mmol, 0.1 eq). The reaction was stirred at 80° C. for 2 h. To the reaction mixture was added brine (150 mL) and EtOAc (100 mL) and the layers were separated. The aqueous layer was extracted with EtOAc (100 mL×3). The combined organic layers were dried over anhydrous Na₂SO₄, filtered and concentrated under reduced pressure to give a residue. The residue was purified by flash silica gel chromatography (SiO₂, PE/EtOAc=50/1 to 4/1) to give compound 57-3 (4.9 g, crude) as a yellow liquid. LCMS: t_{R}=0.363 min, MS (ESI+) m/z=159.1 [M+1]⁺. ¹H NMR (400 MHz, CDCl₃): δ=7.32 (d, J=8.8 Hz, 1H), 6.53-6.41 (m, 2H), 5.21 (m, 1H), 5.13 (s, 1H), 4.11 (s, 2H), 4.11 (s, 2H), 2.10-2.01 (m, 3H).

Procedure for preparation of compound 57-4. A mixture of compound 57-3 (4.8 g, 30.34 mmol, 1 eq), Mn(dpm)₃ (1.83 g, 3.03 mmol, 0.1 eq) and PhSiH₃ (6.57 g, 60.68 mmol, 7.49 mL, 2 eq) in DCM (10 mL) and i-PrOH (200 mL) was degassed and purged with O₂ (30.34 mmol, 1 eq) for 3 times, and then the mixture was stirred at 0° C. for 2 h under O₂ (30.34 mmol, 1 eq) (15 psi) atmosphere. The reaction mixture was quenched with 20% aqueous sodium sulfite. The organic volatiles were removed under reduced pressure. The aqueous mixture was extracted with EtOAc (50 mL×3). The combined organic layers were dried over anhydrous Na₂SO₄, filtered and concentrated under reduced pressure to give compound 57-4 (5.8 g, crude) as a black-brown oil. LCMS: t_{R}=0.585 min, MS (ESI+) m/z=177.1 [M+1]⁺.

Procedure for preparation of compound 56-5. To a solution of compound 57-4 (5.8 g, 32.91 mmol, 1 eq) in DMF (60 mL) and H₂O (60 mL) was added NaHCO₃ (11.06 g, 131.66 mmol, 5.12 mL, 4 eq). The mixture was stirred at 80° C. for 12 h. The reaction mixture was washed with brine (100 mL), and extracted with EtOAc (100 mL×3). The combined organic layers were dried over anhydrous Na₂SO₄, filtered and concentrated under reduced pressure to give a residue. The residue was purified by flash silica gel chromatography (SiO₂, DCM/MeOH=100/1 to 10/1) to give compound 56-5 (1.3 g, 7.34 mmol, 22.29% yield) as a yellow oil. LCMS: t_{R}=0.375 min, MS (ESI+) m/z=178.0 [M+1]⁺. ¹H NMR (400 MHz, CDCl₃): δ=7.63 (d, J=8.4 Hz, 1H), 6.70 (d, J=8.0 Hz, 1H), 6.52 (s, 1H), 4.26 (s, 2H), 1.61 (s, 6H).

Procedure for preparation of compound 56-7. To a solution of compound 56-5 (500 mg, 2.82 mmol, 1 eq) and compound 56-6 (907.90 mg, 2.82 mmol, 1 eq) in NMP (3 mL) was added HCl/dioxane (4 M, 1.06 mL, 1.5 eq). The mixture was stirred at 80° C. for 12 h. The reaction was cooled to room temperature, quenched by addition of triethylamine (1 mL), and diluted with EtOAc (20 mL) and ethanol (1 mL). The mixture was washed with brine (20 mL×3), dried over Na₂SO₄, filtered and concentrated under reduced pressure to give a residue. The residue was purified by flash silica gel chromatography (SiO₂, DCM/MeOH=100/1 to 10/1) to give compound 56-7 (1.13 g, 2.15 mmol, 76.20% yield) as a yellow solid. LCMS: t_{R}=0.445 min, MS (ESI+) m/z=463.0 [M+1]⁺. ¹H NMR (400 MHz, DMSO-d₆): δ=10.31 (s, 1H), 9.00 (d, J=7.6 Hz, 1H), 8.68 (s, 1H), 7.95 (s, 1H), 7.73-7.56 (m, 2H), 7.44-7.35 (m, 4H), 7.30-7.20 (m, 1H), 5.38-5.27 (m, 1H), 5.19 (t, J=4.8 Hz, 1H), 4.31 (m, 2H), 3.94-3.84 (m, 1H), 3.84-3.76 (m, 1H), 1.57 (s, 3H), 1.51 (s, 3H), 1.34 (t, J=6.2 Hz, 3H).

Procedure for preparation of compound 56-8. To a solution of compound 56-7 (1 g, 2.16 mmol, 1 eq) in EtOH (50 mL) was added N₂H₄·H₂O (1.320 g, 26.37 mmol, 1.28 mL, 12.20 eq) slowly at 20° C. The mixture was stirred at 70° C. for 3 h. After 36 h, added N₂H₄·H₂O (1.32 g, 26.38 mmol, 1.28 mL, 12.2 eq) at 20° C., and the mixture was stirred at 80° C. for 12 h. The residue was purified by prep-HPLC (10%-40% ACN in water (0.05% HCl), 20 min) and lyophilized to give compound 56-8 (410 mg, 914.21 μmol, 42.28% yield) as a yellow solid. LCMS: t_{R}=0.344 min, MS (ESI+) m/z=449.2 [M+1]⁺.

Procedure for preparation of compound 57-10. To a solution of compound 57-9 (273.27 mg, 1.00 mmol, 1.5 eq) in DCM (10 mL) was added DCC (138.02 mg, 668.94 μmol, 135.31 uL, 1 eq) and HOBt (90.39 mg, 668.94 μmol, 1 eq) and stirred at 0° C. for 0.5 h, then compound 56-8 (300 mg, 668.94 μmol, 1 eq) was added to the reaction mixture and the resulting mixture was warmed to 25° C. and stirred at 25° C. for another 1.5 h. The reaction mixture was concentrated under reduced pressure to give a residue. The residue was purified by prep-TLC (DCM/MeOH=100/1 to 10/1) to give compound 57-10 (240 mg, 341.49 μmol, 51.05% yield) as a yellow solid. LCMS: t_{R}=0.372 min, MS (ESI+) m/z=703.5 [M+1]⁺. ¹H NMR (400 MHz, DMSO-d₆): δ=10.25-10.08 (m, 2H), 9.83-9.72 (m, 1H), 9.54 (d, J=8.3 Hz, 1H), 8.67 (s, 1H), 7.97 (s, 1H), 7.81 (d, J=8.1 Hz, 1H), 7.72-7.65 (m, 1H), 7.65-7.60 (m, 1H), 7.54 (d, J=8.1 Hz, 1H), 7.34-7.21 (m, 3H), 5.34-5.25 (m, 1H), 5.15 (s, 1H), 4.21-4.00 (m, 3H), 3.89-3.73 (m, 3H), 3.17 (s, 6H), 2.29 (d, J=2.8 Hz, 2H), 2.26-2.20 (m, 4H), 1.77-1.69 (m, 2H), 1.64 (dt, J=4.4, 7.1 Hz, 2H), 1.40 (d, J=1.5 Hz, 9H).

### Procedure for Preparation of Compound 57-11.

To a solution of compound 57-10 (100 mg, 142.29 μmol, 1 eq) in DCM (2 mL) was added TosCl (27.13 mg, 142.29 μmol, 1 eq) followed by TEA (21.60 mg, 213.43 μmol, 29.71 uL, 1.5 eq) and the mixture was stirred at 25° C. for 12 h. The reaction mixture was concentrated under reduced pressure to give a residue. The residue was purified by prep-TLC (DCM/MeOH=100/1 to 10/1) to give compound 57-11 (70 mg, 102.22 μmol, 35.92% yield) as a yellow solid. LCMS: t_{R}=0.409 min, MS (ESI+) m/z=685.5 [M+1]⁺. ¹H NMR (400 MHz, DMSO-d₆): δ=8.98 (d, J=4.4 Hz, 1H), 8.53 (s, 1H), 7.71-7.66 (m, 2H), 7.44-7.41 (m, 1H), 7.35-7.33 (m, 4H), 7.25-7.23 (m, 1H), 5.49-5.44 (m, 1H), 3.98 (d, J=5.2 Hz, 1H), 3.31 (s, 4H), 2.96-2.91 (m, 2H), 2.43-2.40 (m, 2H), 2.32-2.31 (m, 4H), 1.59 (d, J=2.8 Hz, 1H), 1.38 (s, 9H), 1.18-0.80 (m, 3H), 0.78-0.76 (m, 4H).

Procedure for preparation of compound 57-12. To a solution of compound 57-11 (60 mg, 87.62 μmol, 1 eq) in DCM (2 mL) was added HCl/dioxane (4 M, 43.81 uL, 2 eq) and the mixture was stirred at 20° C. for 5 min. The reaction mixture was concentrated under reduced pressure to give compound 57-12 (60 mg, crude, HCl) as a white solid, which was used for next step without further purification.

Procedure for preparation of compound 57-16. A mixture of compound 57-15 (commercially obtained, 3 g, 10.86 mmol, 1 eq), compound 57-14 (2.20 g, 10.86 mmol, 2.26 mL, 1 eq), DIEA (4.21 g, 32.58 mmol, 5.68 mL, 3 eq) and DMAc (15 mL) was stirred at 90° C. for 2 h. To the mixture was added brine (50 mL). The mixture was extracted with ethyl acetate (50 mL×3). The combined organic extracts were washed with brine (50 mL), dried, filtered and concentrated. The residue was purified by flash silica gel chromatography (SiO₂, PE/EA=50/1 to 1/1) in petroleum ether to give compound 57-16 (2.7 g, 5.89 mmol, 54.22% yield) as a green oil. LCMS: t_{R}=0.469 min, MS (ESI+) m/z=359.1 [M+1]⁺. ¹H NMR (400 MHz, DMSO-d₆): δ=11.08 (s, 1H), 7.59 (t, J=7.6 Hz, 1H), 7.10 (d, J=8.8 Hz, 1H), 7.02 (d, J=7.2 Hz, 1H), 6.78 (d, J=5.2 Hz, 1H), 6.53 (t, J=6.0 Hz, 1H), 5.06 (dd, J=12.8, 5.2 Hz, 1H), 3.28 (d, J=6.4 Hz, 1H), 2.91-2.88 (m, 2H), 2.60-2.56 (m, 2H), 2.04-1.98 (m, 1H), 1.58-1.54 (m, 2H), 1.40-1.31 (m, 15H).

Procedure for preparation of compound 57-17. To a solution of compound 57-16 (2.4 g, 5.23 mmol, 1 eq) in DCM (15 mL) was added HCl/dioxane (4 M, 5.23 mL, 4 eq). The mixture was stirred at 20° C. for 1 h. The reaction mixture was filtered and the filter residue was concentrated under the reduced pressure to give compound 57-17 (1.34 g, 3.39 mmol, 64.83% yield, HCl) as a green solid. LCMS: t_{R}=0.459 min, MS (ESI+) m/z=359.1 [M+1]⁺. ¹H NMR (400 MHz, DMSO-d₆): δ=11.08 (s, 1H), 8.06 (s, 3H), 7.58 (dd, J=8.8, 7.2 Hz, 1H), 7.20-6.97 (m, 2H), 5.04 (dd, J=12.8, 5.2 Hz, 1H), 3.30 (t, J=6.8 Hz, 2H), 2.92-2.83 (m, 1H), 2.77-2.70 (m, 2H), 2.65-2.56 (m, 1H), 2.53 (d, J=4.4 Hz, 1H), 2.07-1.97 (m, 1H), 1.59 (m, 4H), 1.45-1.32 (m, 2H).

Procedure for preparation of compound 57-13. To a solution of compound 57-18 (330 mg, 3.49 mmol, 392.86 uL, 1.38 eq) in DMAc (10 mL) was added HATU (1.06 g, 2.79 mmol, 1.10 eq) at 0° C. The mixture was stirred at 0° C. for 1 h. To the cold solution was added compound 57-17 (1.00 g, 2.53 mmol, 1 eq, HCl) followed by DIEA (327.31 mg, 2.53 mmol, 441.12 uL, 1 eq). The reaction was stirred at 20° C. for 0.5 h. To the reaction was added brine (50 mL) and ethyl acetate (50 mL) and the layers were separated. The aqueous layer was extracted with ethyl acetate (30 mL×2). The combined organic extracts were washed with brine (50 mL×2), dried, filtered and concentrated. The residue was purified by prep-HPLC (22%-52% ACN in water (0.225% FA), 20 min) and lyophilized to give compound 57-13 (380 mg, 873.82 μmol, 34.50% yield) as a yellow solid. LCMS: t_{R}=0.397 min, MS (ESI+) m/z=435.0 [M+1]⁺. ¹H NMR (400 MHz, DMSO-d₆): δ=11.09 (s, 1H), 8.19 (s, 1H), 7.59 (t, J=8.4 Hz, 1H), 7.10 (d, J=8.4 Hz, 1H), 7.03 (d, J=7.2 Hz, 1H), 6.54 (t, J=6.0 Hz, 1H), 5.05 (d, J=12.8, 5.2 Hz, 1H), 4.03 (s, 2H), 3.30-3.23 (m, 2H), 3.10 (m, 2H), 2.89-2.80 (m, 1H), 2.65-2.53 (m, 2H), 2.11-2.01 (m, 1H), 1.59 (m, 2H), 1.47 (m, 2H), 1.40-1.30 (m, 2H).

Procedure for preparation of MS-5-007. To a solution of compound 57-12 (25 mg, 40.25 μmol, 1 eq, HCl) in DMF (1 mL) was added DIEA (10.40 mg, 80.50 μmol, 14.02 uL, 2 eq) and compound 57-13 (17.50 mg, 40.25 μmol, 1 eq). The mixture was stirred at 50° C. for 2 h. The residue was purified by prep-HPLC (20%-50% ACN in water (0.225% FA), 9 min) and lyophilized to give MS-5-007 (13.13 mg, 11.84 μmol, 29.42% yield, 92.8% purity, FA) as a yellow solid. LCMS: t_{R}=0.426 min, MS (ESI+) m/z=984.7 [M+1]⁺. ¹H NMR (400 MHz, DMSO-d₆): δ=11.08 (s, 1H), 10.27 (s, 1H), 8.90 (d, J=7.2 Hz, 1H), 8.65 (s, 1H), 8.23 (s, 1H), 7.97 (d, J=1.2 Hz, 1H), 7.73-7.68 (m, 1H), 7.65-7.55 (m, 3H), 7.43-7.34 (m, 4H), 7.29-7.23 (m, 1H), 7.08 (d, J=8.4 Hz, 1H), 7.01 (d, J=7.2 Hz, 1H), 6.54-6.49 (m, 1H), 5.43 (s, 1H), 5.23 (s, 1H), 5.04 (dd, J=12.8, 5.2 Hz, 1H), 3.97-3.80 (m, 2H), 3.28-3.23 (m, 4H), 3.08 (d, J=6.0 Hz, 2H), 2.94 (t, J=6.4 Hz, 4H), 2.81 (s, 2H), 2.60 (d, J=2.0 Hz, 2H), 2.39 (d, J=6.4 Hz, 4H), 2.05-2.00 (m, 1H), 1.99-1.86 (m, 3H), 1.65-1.50 (m, 9H), 1.46-1.41 (m, 2H), 1.34-1.28 (m, 2H), 0.97 (s, 1H).

### Example 3

### Synthesis of MS-5-008

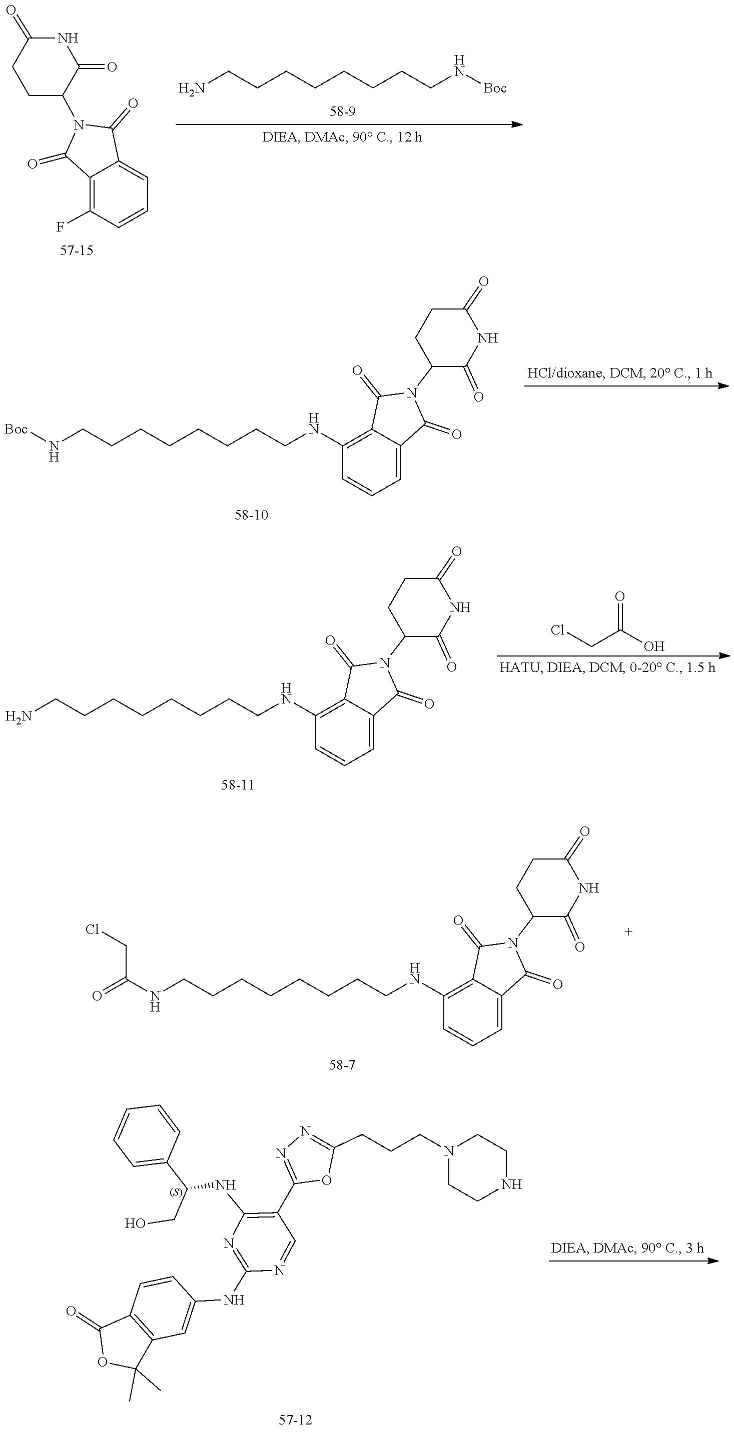

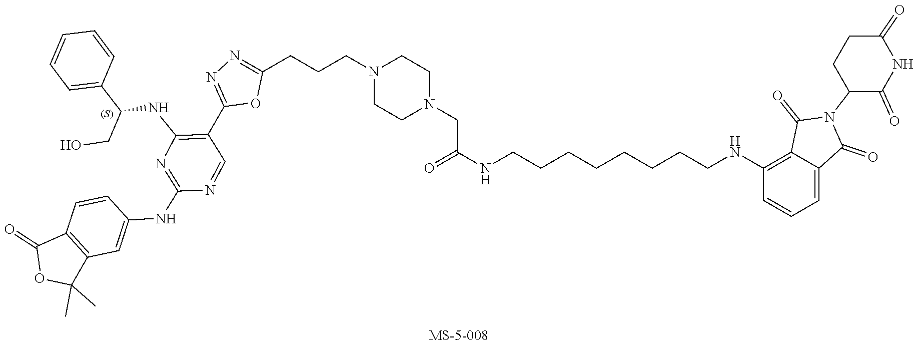

The procedure for preparing 57-12 is described in the synthesis of MS-5-007.

Procedure for preparation of compound 58-10. A mixture of compound 57-15 (commercially obtained, 2 g, 7.24 mmol, 1 eq), compound 58-9 (1.77 g, 7.24 mmol, 1 eq), DIEA (2.81 g, 21.72 mmol, 3.78 mL, 3 eq) and DMAc (15 mL) was stirred at 90° C. for 12 h. LCMS (EC6213-3-P1B) showed the desired mass. The mixture was added brine (50 mL). The mixture was extracted with ethyl acetate (50 mL×3) and the combined organic layers were washed with brine (50 mL), dried, filtered and concentrated. The crude product was purified by flash column chromatography (12% premixed solvent of ethyl acetate/ethanol (ratio: 3:1) in petroleum ether to give compound 58-10 (1.7 g, 3.40 mmol, 46.90% yield) as a green oil. LCMS: t_{R}=0.528 min, MS (ESI+) m/z=501.2[M+1]⁺.

Procedure for preparation of compound 58-11. To a solution of compound 58-10 (1.5 g, 3.00 mmol, 1 eq) in DCM (9 mL) was added HCl/dioxane (4 M, 3.00 mL, 4 eq). The mixture was stirred at 20° C. for 1 h. LCMS showed reactant was consumed and the desired mass was detected. The reaction mixture was filtered and the filter residue was concentrated under the reduced pressure to give compound 58-11 (0.75 g, 1.72 mmol, 57.28% yield, HCl) as a green solid. LCMS: t_{R}=0.349 min, MS (ESI+) m/z=401.2[M+1]⁺. ¹H NMR (400 MHz, DMSO-d₆): δ=11.08 (s, 1H), 7.92 (s, 3H), 7.58 (dd, J=8.4 Hz, 1H), 7.09 (d, J=8.4 Hz, 1H), 7.02 (d, J=7.2 Hz, 1H), 5.05 (d, J=7.6 Hz, 1H), 3.29 (t, J=6.8 Hz, 2H), 2.92-2.82 (m, 1H), 2.77-2.67 (m, 2H), 2.64-2.55 (m, 1H), 2.53 (d, J=5.6 Hz, 1H), 2.08-1.99 (m, 1H), 1.56 (m, 4H), 1.40-1.27 (m, 8H).

Procedure for preparation of compound 58-7. To a solution of Chloroacetic acid (182 mg, 1.93 mmol, 216.67 uL, 1.20 eq) in DMAC (7 mL) was added HATU (664 mg, 1.75 mmol, 1.09 eq) at 0° C. The mixture was stirred at 0° C. for 1 h. Then compound 58-11 (700 mg, 1.60 mmol, 1 eq, HCl) followed by DIEA (207.05 mg, 1.60 mmol, 279.05 uL, 1 eq) was added. The reaction was stirred at 20° C. for 0.5 h. To the reaction was added brine (50 mL) and ethyl acetate (50 mL) and the layers were separated. The aqueous layer was extracted with ethyl acetate (30 mL×2). The combined organic extracts were washed with brine (50 mL×2), dried, filtered and concentrated.

The residue was purified by prep-HPLC (33%-63% ACN in water (0.225% FA), 20 min) and lyophilized to give compound 58-7 (320 mg, 670.93 μmol, 41.88% yield) as a yellow solid. LCMS: t_{R}=0.462 min, MS (ESI+) m/z=477.2[M+1]⁺. ¹H NMR (400 MHz, DMSO-d₆): δ=11.09 (s, 1H), 8.16 (s, 1H), 7.59 (t, J=8.0 Hz, 1H), 7.23-6.82 (m, 2H), 6.52 (t, J=5.6 Hz, 1H), 5.05 (dd, J=12.8, 5.2 Hz, 1H), 4.02 (s, 2H), 3.29 (s, 2H), 3.07 (m, 2H), 2.96-2.78 (m, 1H), 2.65-2.54 (m, 2H), 2.10-2.01 (m, 1H), 1.67-1.51 (m, 2H), 1.44-1.24 (m, 10H).

Procedure for preparation of compound MS-5-008. To a solution of compound 58-6 (26.05 mg, 41.93 μmol, 1 eq, HCl) in DMF (2 mL) was added DIEA (16.26 mg, 125.80 μmol, 21.91 uL, 3 eq) and compound 58-7 (20 mg, 41.93 μmol, 1 eq) and the mixture was stirred at 90° C. for 3 h. LCMS showed desired m/z. The reaction mixture was filtered to give a residue. The residue was purified by prep-HPLC (26%-56% ACN in water (0.225% FA), 9 min) to give compound MS-5-008 (9.75 mg, 8.94 μmol, 21.32% yield, 94% purity) as a yellow solid. LCMS: t_{R}=0.448 min, MS (ESI+) m/z=1025.5[M+1]⁺. ¹H NMR (400 MHz, DMSO-d₆): δ=11.09 (s, 1H), 10.27 (s, 1H), 8.91 (d, J=8.0 Hz, 1H), 8.65 (s, 1H), 8.14 (s, 1H), 7.97 (s, 1H), 7.75-7.69 (m, 1H), 7.66-7.54 (m, 3H), 7.44-7.34 (m, 4H), 7.30-7.23 (m, 1H), 7.08 (d, J=8.4 Hz, 1H), 7.02 (d, J=6.8 Hz, 1H), 6.59-6.42 (m, 1H), 5.52-5.37 (m, 1H), 5.32-5.19 (m, 1H), 5.05 (dd, J=5.6, 12.8 Hz, 1H), 3.97-3.89 (m, 1H), 3.88-3.81 (m, 1H), 3.10-3.01 (m, 3H), 2.95 (t, J=7.2 Hz, 3H), 2.84 (s, 2H), 2.44-2.37 (m, 6H), 2.08-1.88 (m, 4H), 1.65-1.50 (m, 9H), 1.45-1.15 (m, 13H).

### Example 4

### Synthesis of MS-5-009

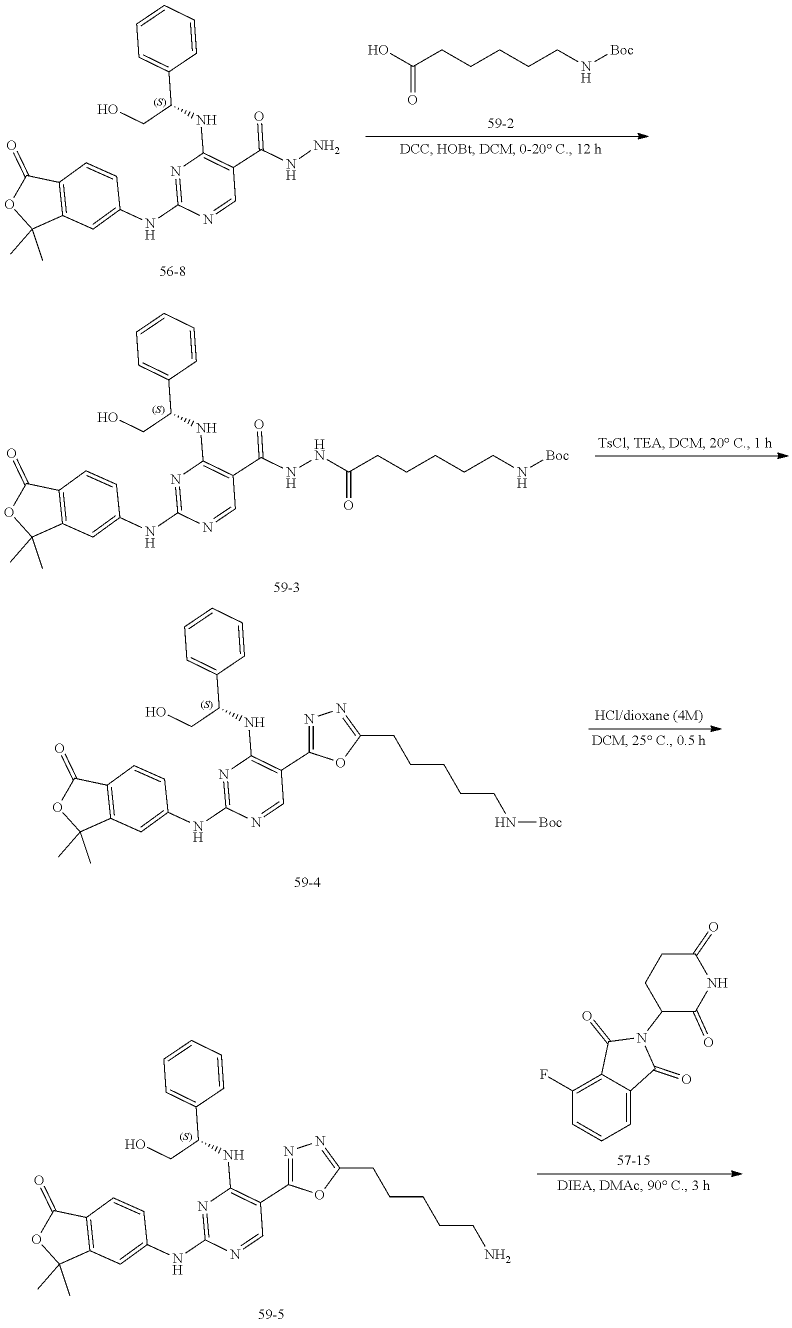

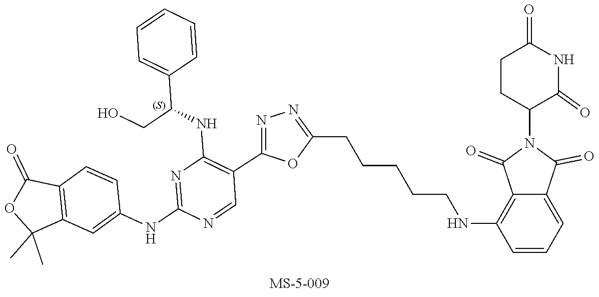

The procedure for preparing 56-8 is described in the synthesis of MS-5-006.

Procedure for preparation of compound 59-3. To a solution of compound 59-2 (270.75 mg, 1.17 mmol, 1.5 eq) in DCM (10.5 mL) was added DCC (161.02 mg, 780.43 μmol, 157.87 uL, 1 eq) and HOBt (105.45 mg, 780.43 μmol, 1 eq) at 0° C. for 0.5 h, then was added compound 56-8 (350 mg, 780.43 μmol, 1 eq). The mixture was stirred at 20° C. for 11.5 h. The reaction mixture was filtered and the filtrate was concentrated under the reduced pressure to give a residue. The residue was purified by prep-HPLC (26%-56% ACN in water (0.05% HCl), 10 min) and lyophilized to give compound 59-3 (135 mg, 204.01 μmol, 26.14% yield) as a black brown solid. LCMS (EC6212-53-P1B): t_{R}=0.428 min, MS (ESI+) m/z=662.2 [M+1]⁺. ¹H NMR (EC6212-53-P1A) (400 MHz, DMSO-d₆): δ=10.41-10.18 (m, 2H), 9.81 (s, 1H), 9.67 (d, J=7.6 Hz, 1H), 8.67 (s, 1H), 7.94 (s, 1H), 7.76-7.57 (m, 2H), 7.47-7.31 (m, 4H), 7.26 (m, 1H), 6.79 (s, 1H), 5.29 (m, 1H), 3.83 (d, J=4.4 Hz, 1H), 3.79-3.74 (m, 1H), 2.91 (q, J=6.4 Hz, 2H), 2.19 (t, J=7.2 Hz, 2H), 1.56 (d, J=16.0 Hz, 8H), 1.46-1.22 (m, 13H).

Procedure for preparation of compound 59-4. To a solution of compound 59-3 (135 mg, 204.01 μmol, 1 eq) in DCM (1.5 mL) was added TsCl (38.89 mg, 204.01 μmol, 1 eq) and TEA (30.96 mg, 306.01 μmol, 42.59 uL, 1.5 eq). The mixture was stirred at 20° C. for 1 h. The reaction mixture was concentrated under the reduced pressure to give a residue. The residue was purified by prep-HPLC (50%-80% ACN in water (0.05% HCl), 8 min) and lyophilized to give compound 59-4 (174 mg, crude) as a yellow solid. LCMS: t_{R}=0.506 min, MS (ESI+) m/z=644.2 [M+1]⁺. ¹H NMR (400 MHz, DMSO-d₆): δ=10.32 (s, 1H), 8.93 (d, J=7.6 Hz, 1H), 8.69 (s, 1H), 8.02 (s, 1H), 7.80-7.71 (m, 1H), 7.71-7.63 (m, 1H), 7.53-7.37 (m, 4H), 7.36-7.25 (m, 1H), 6.83 (t, J=5.2 Hz, 1H), 5.53-5.43 (m, 1H), 5.27 (t, J=4.8 Hz, 1H), 4.01-3.85 (m, 2H), 3.05-2.92 (m, 4H), 1.82 (m, 2H), 1.60 (d, J=19.6 Hz, 6H), 1.50-1.35 (m, 13H).

Procedure for preparation of compound 59-5. To a solution of compound 59-4 (90 mg, 139.81 μmol, 1 eq) in DCM (1 mL) was added HCl/dioxane (4 M, 139.81 uL, 4 eq). The mixture was stirred at 25° C. for 0.5 h. The reaction mixture was filtered and the filter residue was concentrated under the reduced pressure to give compound 59-5 (76 mg, crude, HCl) as a yellow solid. LCMS: t_{R}=0.375 min, MS (ESI+) m/z=544.2 [M+1]⁺.

Procedure for preparation of MS-5-009. To a solution of compound 59-5 (76 mg, 131.02 μmol, 1 eq, HCl) and compound 57-15 (40.40 mg, 146.27 μmol, 1 eq) in DMAc (3 mL) was added DIEA (50.80 mg, 393.05 μmol, 68.46 uL, 3 eq). The mixture was stirred at 90° C. for 3 h. The residue was purified by prep-HPLC (46%-76% ACN in water (0.225% FA), 9 min) and lyophilized to give MS-5-009 (22.64 mg, 26.75 μmol, 20.42% yield, 94.5% purity) as a yellow solid. LCMS: t_{R}=0.675 min, MS (ESI+) m/z=800.4 [M+1]⁺. ¹H NMR (400 MHz, DMSO-d₆): δ=11.08 (s, 1H), 10.27 (s, 1H), 8.88 (d, J=7.6 Hz, 1H), 8.64 (s, 1H), 7.98 (d, J=1.2 Hz, 1H), 7.74-7.68 (m, 1H), 7.67-7.62 (m, 1H), 7.58 (dd, J=8.4, 6.2 Hz, 1H), 7.44-7.33 (m, 4H), 7.31-7.23 (m, 1H), 7.12 (d, J=8.4 Hz, 1H), 7.02 (d, J=6.8 Hz, 1H), 6.58 (t, J=6.0 Hz, 1H), 5.47-5.39 (m, 1H), 5.22 (t, J=4.8 Hz, 1H), 5.04 (dd, J=12.8, 5.2 Hz, 1H), 3.97-3.88 (m, 1H), 3.87-3.80 (m, 1H), 3.39-3.33 (m, 2H), 3.04-2.94 (m, 2H), 2.92-2.82 (m, 1H), 2.59 (d, J=2.4 Hz, 2H), 2.06-1.98 (m, 1H), 1.84 (m, 2H), 1.71-1.61 (m, 2H), 1.58 (s, 3H), 1.53 (s, 3H), 1.51-1.44 (m, 2H).

### Example 5

### Synthesis of MS-5-010

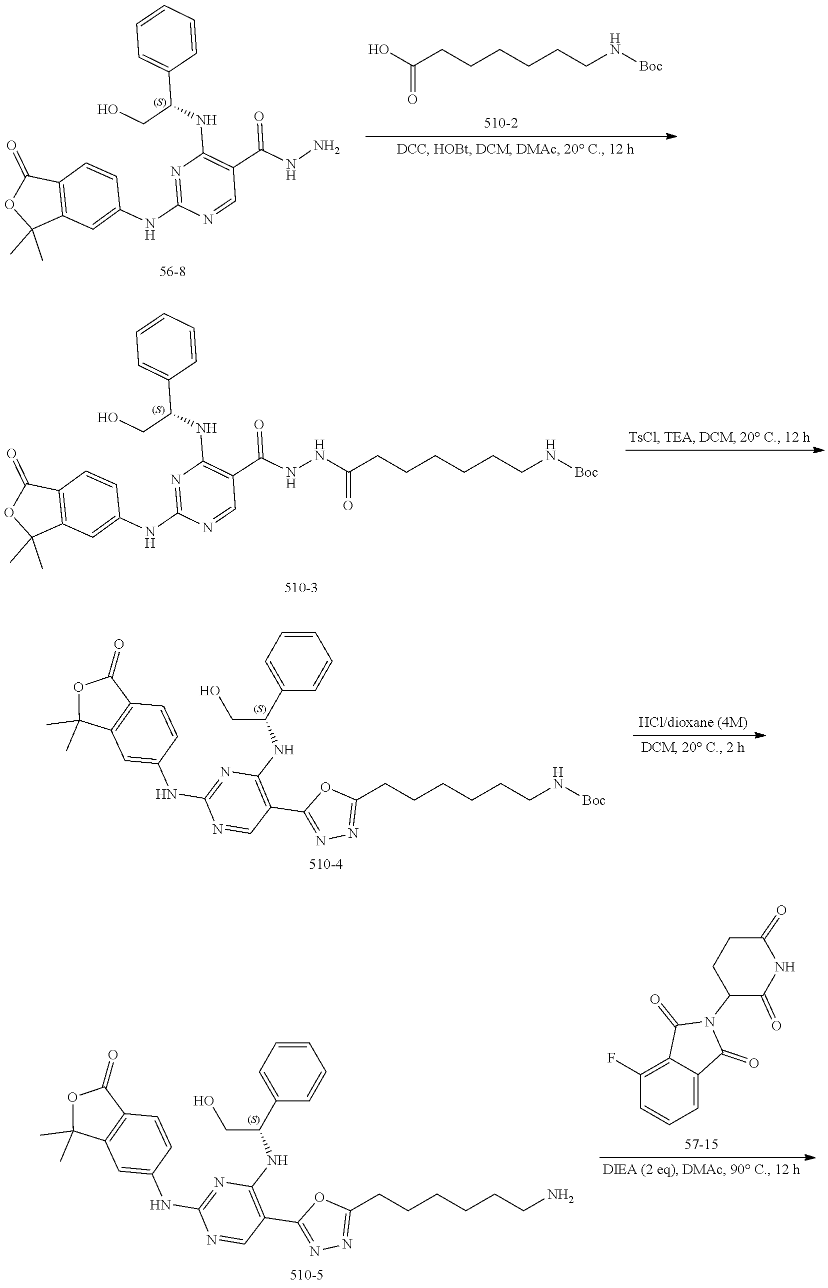

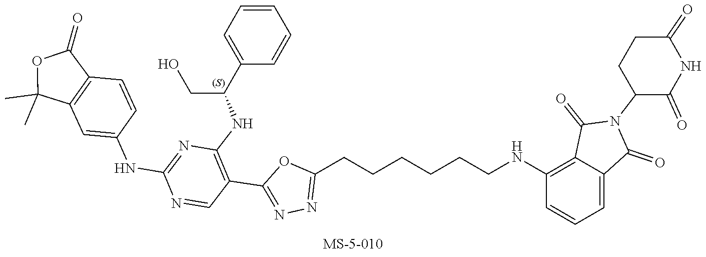

The procedure for preparing 56-8 is described in the synthesis of MS-5-006.

Procedure for preparation of compound 510-3. To a solution of compound 510-2 (191.45 mg, 780.43 μmol, 1 eq) in DCM (10 mL) was added DCC (161.02 mg, 780.43 μmol, 157.87 uL, 1 eq) and HOBt (105.45 mg, 780.43 μmol, 1 eq), the mixture was stirred at 0° C. for 0.5 h, then compound 56-8 (350 mg, 780.43 μmol, 1 eq) was added and the mixture was warmed to 25° C. and stirred at 25° C. for another 1.5 h. The reaction mixture was filtered. The residue was purified by Prep-TLC (SiO₂, Ethyl acetate) to give compound 510-3 (200 mg, 295.96 μmol, 37.92% yield) as a brown solid. LCMS: t_{R}=0.442 min, MS (ESI+) m/z=676.2[M+1]⁺. ¹H NMR (400 MHz, DMSO-d₆): δ=10.18 (s, 1H), 10.14 (s, 1H), 9.76 (s, 1H), 9.54 (d, J=8.0 Hz, 1H), 8.67 (s, 1H), 7.96 (d, J=1.2 Hz, 1H), 7.76-7.59 (m, 2H), 7.46-7.20 (m, 5H), 6.84-6.71 (m, 1H), 5.37-5.24 (m, 1H), 5.14 (t, J=4.8 Hz, 1H), 3.93-3.68 (m, 2H), 2.91 (q, J=6.8 Hz, 2H), 2.19 (t, J=7.2 Hz, 2H), 1.76-1.63 (m, 1H), 1.56 (d, J=18.4 Hz, 9H), 1.38 (s, 9H), 1.34-1.24 (m, 5H).

Procedure for preparation of compound 510-4. To a mixture of compound 510-3 (190 mg, 281.16 μmol, 1 eq) and TosCl (53.60 mg, 281.16 μmol, 1 eq) in DCM (4 mL) was added TEA (42.68 mg, 421.74 μmol, 58.70 uL, 1.5 eq) and the mixture was stirred at 25° C. for 1 h. The reaction mixture was filtered. The residue was purified by prep-TLC (EtOAc) to give compound 510-4 (100 mg, 152.03 μmol, 54.07% yield) as a brown solid. LCMS: t_{R}=0.516 min, MS (ESI+) m/z=658.1[M+1]⁺. ¹H NMR (400 MHz, DMSO-d₆): δ=10.38 (s, 1H), 8.98 (d, J=7.6 Hz, 1H), 8.73 (s, 1H), 8.07 (s, 1H), 7.85-7.69 (m, 2H), 7.53-7.42 (m, 4H), 7.39-7.28 (m, 1H), 6.91-6.80 (m, 1H), 5.52 (td, J=4.4, 7.6 Hz, 1H), 5.32 (t, J=4.9 Hz, 1H), 4.09-3.84 (m, 2H), 3.09-2.92 (m, 4H), 1.86 (quin, J=7.2 Hz, 3H), 1.67 (s, 3H), 1.62 (s, 3H), 1.51-1.43 (m, 14H).

Procedure for preparation of compound 510-5. To a solution of Compound 510-4 (90 mg, 136.83 μmol, 1 eq) in DCM (2 mL) was added HCl/dioxane (4 M, 2 mL, 58.47 eq) and the mixture was stirred at 20° C. for 2 h. The reaction mixture was concentrated to give Compound 510-5 (80 mg, 134.66 μmol, 98.41% yield, HCl) as a white solid, which was used for next step without further purification. LCMS: t_{R}=0.379 min, MS (ESI+) m/z=558.2[M+1]⁺.

Procedure for preparation of MS-5-010. To a solution of compound 510-6 (36.10 mg, 130.69 μmol, 1 eq) and compound 510-5 (80 mg, 134.66 μmol, 1.03 eq, HCl) in DMAc (3 mL) was added DIEA (50.67 mg, 392.07 μmol, 68.29 uL, 3 eq) and the mixture was stirred at 90° C. for 12 h. The mixture was filtered and purified by prep-HPLC (49%-79% ACN in water (0.225% FA), 10 min) to give MS-5-010 (FA salt, 7.03 mg, 8.32 μmol, 6.37% yield, 96.34% purity) as a yellow solid. LCMS: t_{R}=0.510 min, MS (ESI+) m/z=814.2[M+1]⁺. ¹H NMR (400 MHz, DMSO-d₆): δ=11.09 (s, 1H), 10.29 (s, 1H), 8.89 (d, J=8.0 Hz, 1H), 8.65 (s, 1H), 8.41 (s, 1H), 7.98 (s, 1H), 7.73-7.69 (m, 1H), 7.66-7.62 (m, 1H), 7.61-7.55 (m, 1H), 7.43-7.35 (m, 4H), 7.30-7.24 (m, 1H), 7.11 (d, J=8.8 Hz, 1H), 7.02 (d, J=7.2 Hz, 1H), 6.56 (t, J=5.6 Hz, 1H), 5.48-5.40 (m, 1H), 5.24 (t, J=4.8 Hz, 1H), 5.05 (dd, J=12.8, 5.2 Hz, 1H), 3.98-3.78 (m, 2H), 3.01-2.82 (m, 4H), 2.61 (d, J=2.4 Hz, 2H), 2.07-2.01 (m, 1H), 1.85-1.78 (m, 2H), 1.66-1.57 (m, 6H), 1.54 (s, 3H), 1.48-1.41 (m, 4H).

### Example 6

### Synthesis of MS-5-011

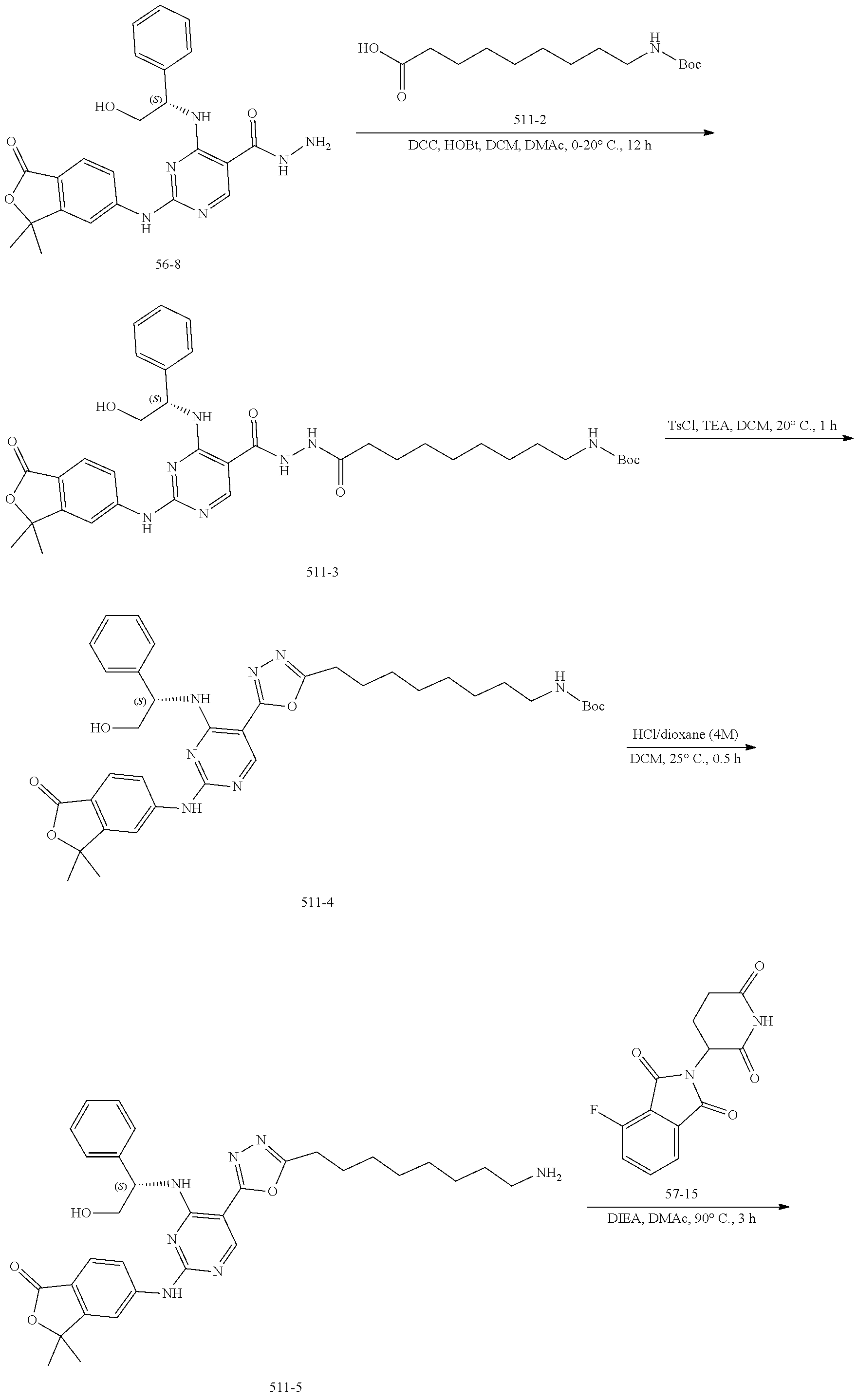

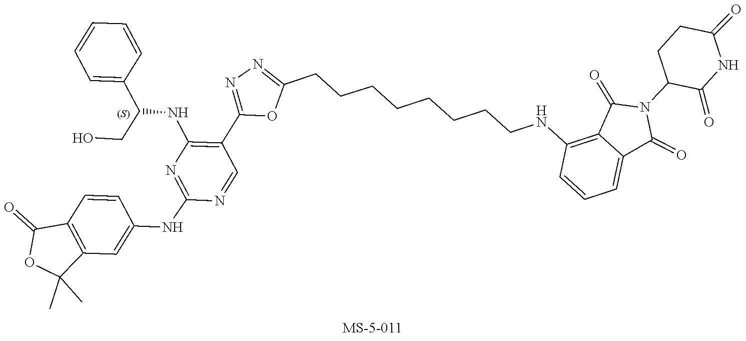

The procedure for preparing 56-8 is described in the synthesis of MS-5-006.

Procedure for preparation of compound 511-3. To a solution of compound 511-2 (320.01 mg, 1.17 mmol, 1.5 eq) in DCM (10.5 mL) was added DCC (161.02 mg, 780.43 μmol, 157.87 uL, 1 eq) and HOBt (105.45 mg, 780.43 μmol, 1 eq) at 0° C. for 0.5 h, then was added compound 56-8 (350 mg, 780.43 μmol, 1 eq). The mixture was stirred at 20° C. for 11.5 h. The reaction mixture was filtered and the filtrate was concentrated under the reduced pressure to give a residue. The residue was purified by prep-HPLC (33%-63% ACN in water (0.05% HCl), 10 min) and lyophilized to give compound 511-3 (344 mg, 488.76 μmol, 62.63% yield) as a black brown solid. LCMS: t_{R}=0.469 min, MS (ESI+) m/z=704.2 [M+1]⁺. ¹H NMR (400 MHz, DMSO-d₆): δ=10.46-10.15 (m, 2H), 9.80 (s, 1H), 9.70 (d, J=7.6 Hz, 1H), 8.67 (s, 1H), 7.93 (s, 1H), 7.71-7.60 (m, 2H), 7.48-7.31 (m, 4H), 7.30-7.19 (m, 1H), 6.75 (br s, 1H), 5.28 (m, 1H), 3.83 (d, J=6.4 Hz, 1H), 3.78-3.73 (m, 1H), 2.91-2.87 (m, 2H), 2.20-2.17 (m, 2H), 1.58 (s, 3H), 1.54 (s, 3H), 1.37 (s, 15H), 1.24 (d, J=10.8 Hz, 6H).

Procedure for preparation of compound 511-4. To a solution of compound 511-3 (340 mg, 483.07 μmol, 1 eq) in DCM (6.5 mL) was added TsCl (92.10 mg, 483.07 μmol, 1 eq) and TEA (73.32 mg, 724.61 μmol, 100.86 uL, 1.5 eq). The mixture was stirred at 20° C. for 1 h. The reaction mixture was filtered and the filtrate was concentrated under the reduced pressure to give a residue. The residue was purified by prep-HPLC (51%-81% ACN in water (0.05% HCl), 8 min) and lyophilized to give compound 511-4 (90 mg, 131.23 μmol, 27.17% yield) as a yellow solid. LCMS: t_{R}=0.547 min, MS (ESI+) m/z=686.1 [M+1]⁺. ¹H NMR (400 MHz, DMSO-d₆): δ=10.29 (s, 1H), 8.89 (d, J=7.6 Hz, 1H), 8.65 (s, 1H), 7.99 (s, 1H), 7.77-7.68 (m, 1H), 7.67-7.60 (m, 1H), 7.48-7.33 (m, 4H), 7.31-7.24 (m, 1H), 6.75 (s, 1H), 5.49-5.40 (m, 1H), 5.24 (t, J=4.8 Hz, 1H), 3.98-3.80 (m, 2H), 3.01-2.84 (m, 4H), 1.78 (td, J=7.3, 14.4 Hz, 2H), 1.56 (d, J=19.5 Hz, 6H), 1.49-1.17 (m, 19H).

Procedure for preparation of compound 511-5. To a solution of compound 511-4 (90 mg, 131.23 μmol, 1 eq) in DCM (1 mL) was added HCl/dioxane (4 M, 131.23 uL, 4 eq). The mixture was stirred at 25° C. for 0.5 h. The reaction mixture was filtered and the filter residue was concentrated under the reduced pressure to give compound 511-5 (91 mg, crude, HCl) as a yellow solid. LCMS (EC6212-71-P1B): t_{R}=0.410 min, MS (ESI+) m/z=586.2 [M+1]⁺.

Procedure for preparation of MS-5-011. To a solution of compound 511-5 (91 mg, 146.27 μmol, 1 eq, HCl) and compound 57-15 (40.40 mg, 146.27 μmol, 1 eq) in DMAc (4 mL) was added DIEA (56.71 mg, 438.80 μmol, 76.43 uL, 3 eq). The mixture was stirred at 90° C. for 3 h. The residue was purified by prep-HPLC (52%-82% ACN in water (0.225% FA), 9 min) and lyophilized to give MS-5-011 (2.67 mg, 3.08 μmol, 2.11% yield, 97.09% purity) as a yellow solid. LCMS: t_{R}=0.536 min, MS (ESI+) m/z=842.2 [M+1]⁺. ¹H NMR (400 MHz, DMSO-d₆): δ=11.08 (s, 1H), 10.28 (s, 1H), 8.88 (d, J=7.6 Hz, 1H), 8.64 (s, 1H), 7.97 (s, 1H), 7.73-7.69 (m, 1H), 7.65-7.61 (m, 1H), 7.60-7.54 (m, 1H), 7.44-7.34 (m, 4H), 7.29-7.23 (m, 1H), 7.08 (d, J=8.4 Hz, 1H), 7.01 (d, J=6.8 Hz, 1H), 6.55-6.49 (m, 1H), 5.46-5.40 (m, 1H), 5.24-5.20 (m, 1H), 5.04 (dd, J=12.4, 4.8 Hz, 1H), 3.95-3.88 (m, 1H), 3.84 (dd, J=10.4, 4.8 Hz, 1H), 3.29-3.25 (m, 2H), 2.97-2.91 (m, 2H), 2.90-2.81 (m, 1H), 2.60 (s, 2H), 2.05-2.00 (m, 1H), 1.81-1.75 (m, 2H), 1.55 (d, J=19.6 Hz, 8H), 1.45-1.33 (m, 8H).

### Example 7

### Synthesis of MS-5-015

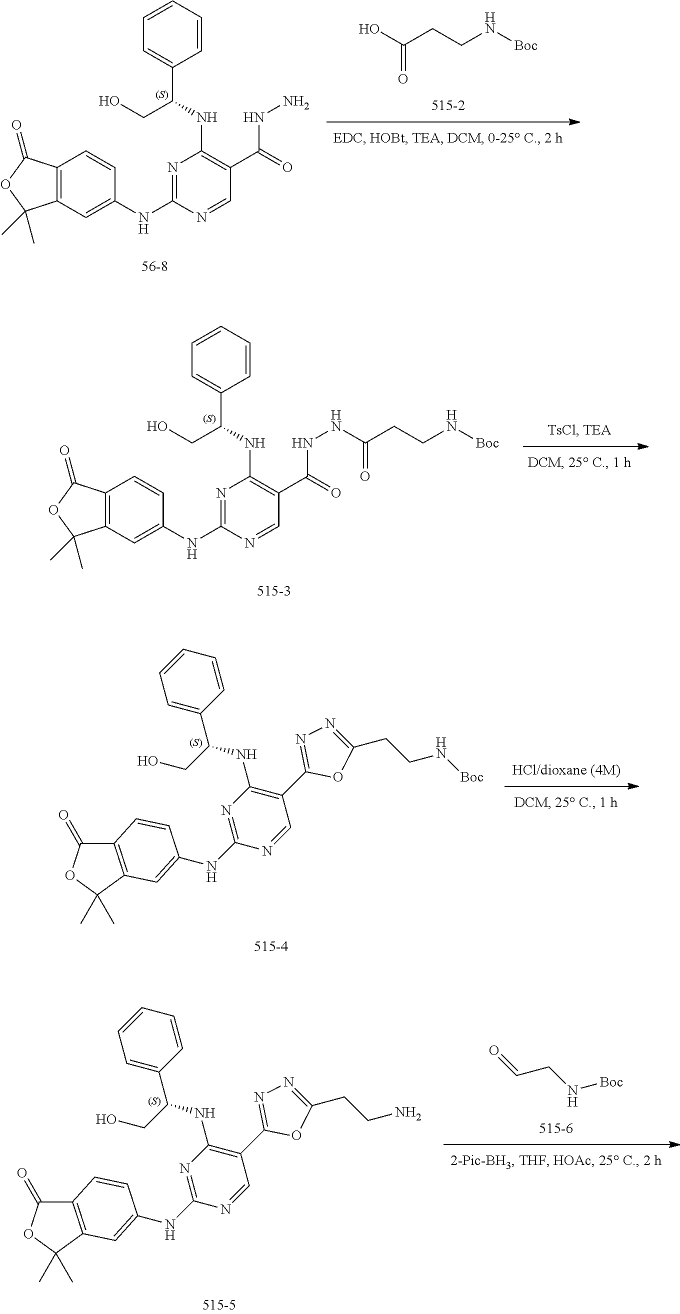

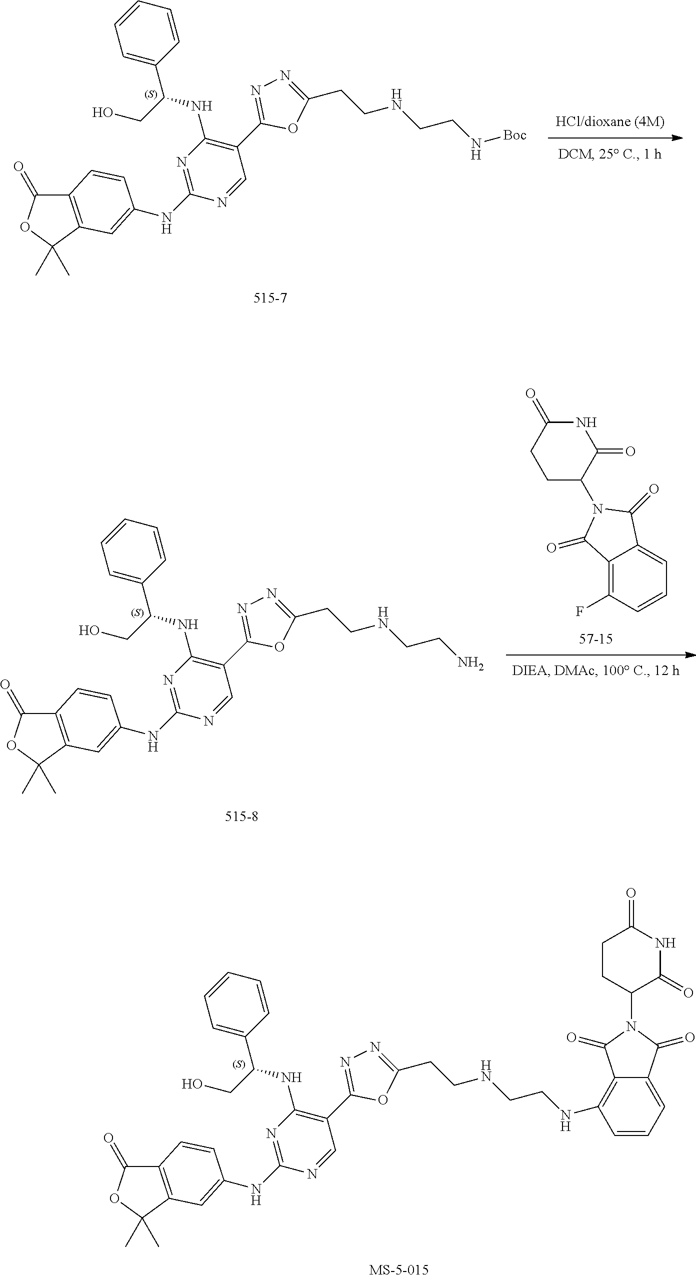

The procedure for preparing 56-8 is described in the synthesis of MS-5-006.

Procedure for preparation of compound 515-3. To a solution of compound 515-2 (1.27 g, 6.69 mmol, 1 eq) in DCM (30 mL) was added EDCI (1.28 g, 6.69 mmol, 1 eq), HOBt (903.89 mg, 6.69 mmol, 1 eq) and TEA (744.58 mg, 7.36 mmol, 1.02 mL, 1.1 eq) at 0° C. for 0.5 h. Then compound 56-8 (3 g, 6.69 mmol, 1 eq) was added, the mixture was stirred at 25° C. for 1.5 h. The reaction mixture was partitioned between DCM (50 mL) and water (50 mL). After quenching the reaction, the reaction mixture was poured into separatory funnel and separated. The residue was purified by prep-HPLC (FA condition) and lyophilized to give compound 515-3 (1.48 g, 2.32 mmol, 34.68% yield, 97% purity) as a white solid. LCMS: t_{R}=0.408 min, MS (ESI+) m/z=620.3 [M+1]⁺. ¹H NMR (400 MHz, DMSO-d₆): δ=10.45-10.00 (m, 2H), 9.84 (s, 1H), 8.66 (s, 1H), 7.96 (s, 1H), 7.75-7.65 (m, 1H), 7.64-7.56 (m, 1H), 7.37-7.34 (m, 3H), 7.25 (m, 1H), 6.81 (t, J=5.2 Hz, 1H), 5.36-5.23 (m, 1H), 5.15 (t, J=4.8 Hz, 1H), 3.89-3.79 (m, 1H), 3.79-3.68 (m, 1H), 3.25-3.08 (m, 2H), 2.37 (t, J=7.6 Hz, 2H), 1.57 (s, 3H), 1.53 (s, 3H), 1.39 (s, 9H).

Procedure for preparation of compound 515-4. To a solution of compound 515-3 (1.4 g, 2.26 mmol, 1 eq) in DCM (14 mL) was added TsCl (473.80 mg, 2.49 mmol, 1.1 eq) and TEA (342.92 mg, 3.39 mmol, 471.69 uL, 1.5 eq). The mixture was stirred at 25° C. for 1 h. The reaction mixture was partitioned between EtOAc (30 mL) and water (30 mL). After quenching the reaction, the reaction mixture was poured into separatory funnel and separated. The residue was purified by flash silica gel chromatography (SiO₂, DCM/MeOH=100/1 to 10/1) to give compound 515-4 (860 mg, 1.43 mmol, 63.27% yield) as a colourless solid. LCMS: t_{R}=0.468 min, MS (ESI+) m/z=602.2 [M+1]⁺. ¹H NMR (400 MHz, DMSO-d₆): δ=10.31 (s, 1H), 8.88 (d, J=8.0 Hz, 1H), 8.64 (s, 1H), 7.98 (s, 1H), 7.75-7.67 (m, 1H), 7.67-7.60 (m, 1H), 7.45-7.32 (m, 4H), 7.31-7.23 (m, 1H), 7.08 (t, J=5.6 Hz, 1H), 5.43 (m, 1H), 5.24 (t, J=4.8 Hz, 1H), 3.98-3.88 (m, 1H), 3.87-3.78 (m, 1H), 3.42-3.35 (m, 2H), 3.11-2.99 (m, 2H), 1.58 (s, 3H), 1.53 (s, 3H), 1.36 (s, 9H).

Procedure for preparation of compound 515-5. To a solution of compound 515-2 (860 mg, 1.43 mmol, 1 eq) in DCM (10 mL) was added HCl/dioxane (4 M, 1.43 mL, 4 eq). The mixture was stirred at 25° C. for 1 h. The reaction mixture was filtered and the filter residue was concentrated under the reduced pressure to give compound 515-3 (2.2 g, crude) as a yellow solid. LCMS: t_{R}=0.357 min, MS (ESI+) m/z=502.0[M+1]⁺. ¹H NMR (400 MHz, DMSO-d₆): δ=10.55 (s, 1H), 9.03 (d, J=8.0 Hz, 1H), 8.74 (s, 1H), 8.33 (s, 3H), 7.95 (s, 1H), 7.76-7.63 (m, 2H), 7.42-7.34 (m, 4H), 7.30-7.22 (m, 1H), 5.53-5.36 (m, 1H), 5.33-5.20 (m, 1H), 3.97-3.88 (m, 1H), 3.88-3.80 (m, 1H), 3.44-3.21 (m, 4H), 1.58 (s, 3H), 1.53 (s, 3H).

Procedure for preparation of compound 515-7. To a solution of compound 515-5 (500 mg, 929.37 μmol, 1 eq, HCl) and compound 515-6 (118.35 mg, 743.50 μmol, 0.8 eq) in THF (5 mL) was added HOAc (167.43 mg, 2.79 mmol, 159.46 uL, 3 eq) and borane;2-methylpyridine (198.81 mg, 1.86 mmol, 2 eq), the mixture was stirred at 25° C. for 2 h. The reaction mixture was quenched by addition ACN (20 mL), and concentrated under the reduced pressure to give a residue. The residue was purified by prep-HPLC (12%-42% ACN in water (0.225% FA), 10 min) and lyophilized to give compound 515-7 (1.52 g, crude) as a yellow solid. LCMS: t_{R}=0.394 min, MS (ESI+) m/z=645.2 [M+1]⁺. ¹H NMR (400 MHz, DMSO-d₆): δ=10.30 (s, 1H), 8.90 (d, J=8.0 Hz, 1H), 8.78-8.53 (m, 1H), 8.19 (s, 1H), 7.98 (s, 1H), 7.82-7.68 (m, 1H), 7.67-7.57 (m, 1H), 7.48-7.33 (m, 4H), 7.31-7.06 (m, 1H), 6.74 (t, J=5.2 Hz, 1H), 5.51-5.38 (m, 1H), 5.27 (s, 1H), 3.94-3.89 (m, 1H), 3.85-3.82 (m, 1H), 3.13-2.96 (m, 6H), 2.60 (t, J=6.4 Hz, 2H), 1.67-1.48 (m, 6H), 1.35 (s, 9H).

Procedure for preparation of compound 515-8. To a solution of compound 515-7 (117 mg, 181.47 μmol, 1 eq) in DCM (1 mL) was added HCl/dioxane (4 M, 136.11 uL, 3 eq). The mixture was stirred at 25° C. for 1 h. The reaction mixture was filtered and the filter residue was concentrated under the reduced pressure to give compound 515-8 (108 mg, crude, HCl) as a black brown solid. LCMS: t_{R}=0.329 min, MS (ESI+) m/z=545.2 [M+1]⁺.

Procedure for preparation of MS-5-015. To a solution of compound 515-8 (108 mg, 185.87 μmol, 1 eq, HCl) and compound 57-15 (51.34 mg, 185.87 μmol, 1 eq) in DMAC (1 mL) was added DIEA (48.04 mg, 371.73 μmol, 64.75 uL, 2 eq). The mixture was stirred at 100° C. for 12 h. The residue was purified by prep-HPLC (16%-46% ACN in water (0.05% HCl), 8 min) and lyophilized to give MS-5-015 (2.59 mg, 2.47 μmol, 1.33% yield, 76.38% purity) as a yellow solid. LCMS: t_{R}=1.236 min, MS (ESI+) m/z=801.4 [M+1]⁺. ¹H NMR (400 MHz, DMSO-d₆): δ=11.07 (s, 1H), 10.26 (s, 1H), 8.88 (d, J=6.0 Hz, 1H), 8.63 (d, J=3.2 Hz, 1H), 7.97 (s, 1H), 7.77-7.67 (m, 1H), 7.66-7.61 (m, 1H), 7.60-7.52 (m, 1H), 7.46-7.32 (m, 4H), 7.30-7.22 (m, 1H), 7.11 (d, J=8.8 Hz, 1H), 7.01 (d, J=7.2 Hz, 1H), 6.83-6.66 (m, 1H), 5.51-5.34 (m, 1H), 5.28-5.16 (m, 1H), 4.99 (dd, J=12.8, 5.2 Hz, 1H), 3.98-3.87 (m, 1H), 3.87-3.78 (m, 1H), 3.09 (d, J=4.4 Hz, 4H), 3.02-2.70 (m, 4H), 2.08-1.85 (m, 2H), 1.58 (s, 3H), 1.53 (s, 3H).

### Example 8

### Synthesis of MS-5-016

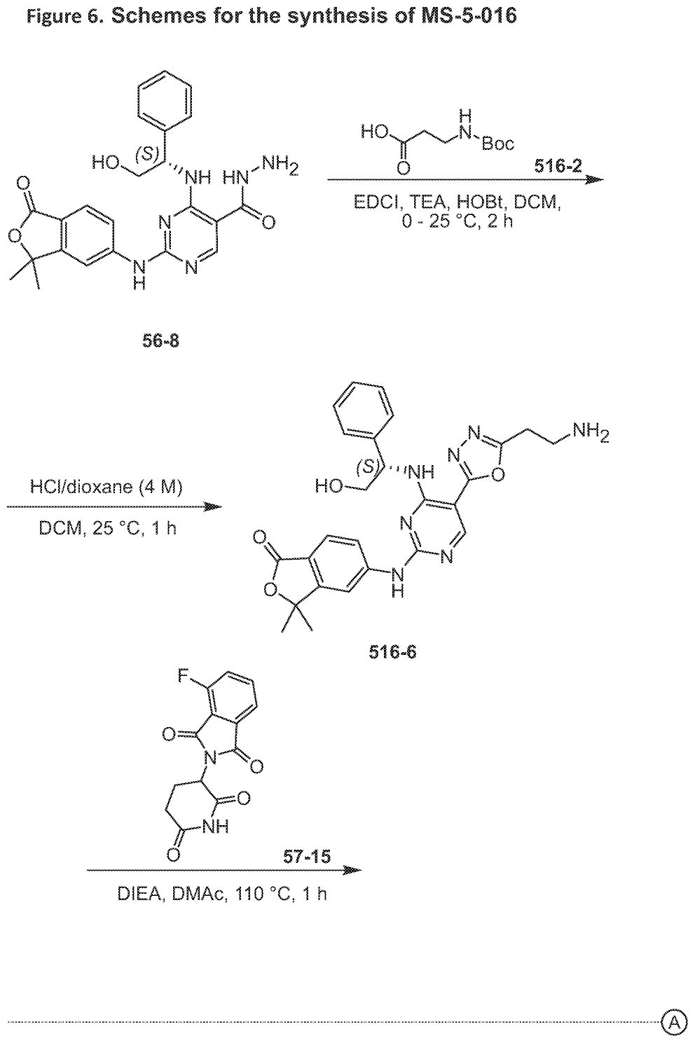

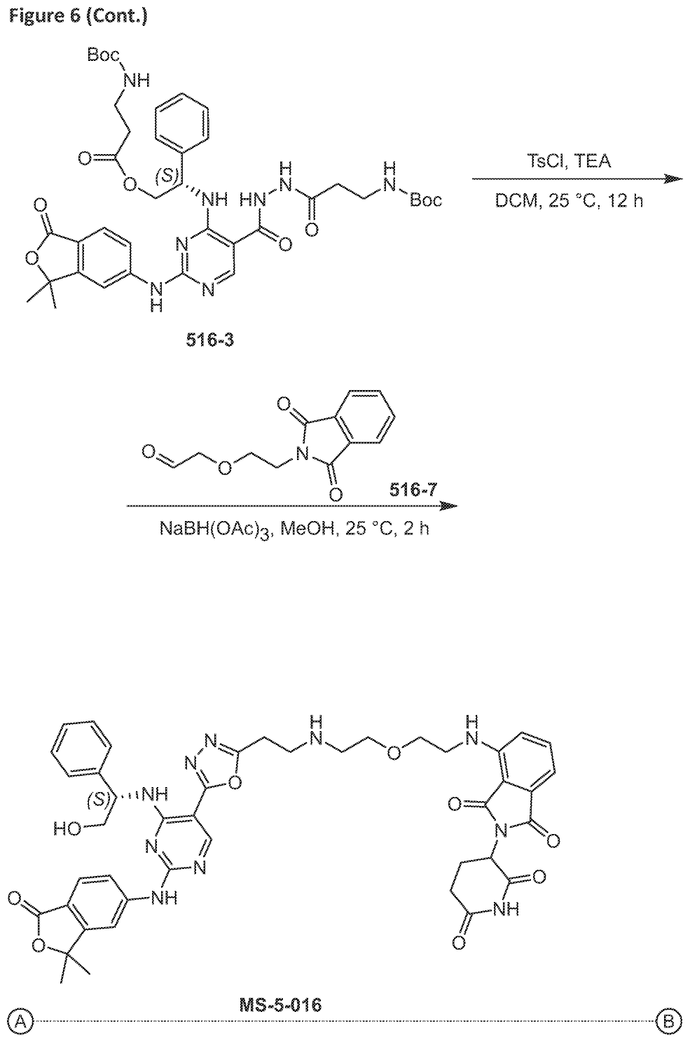

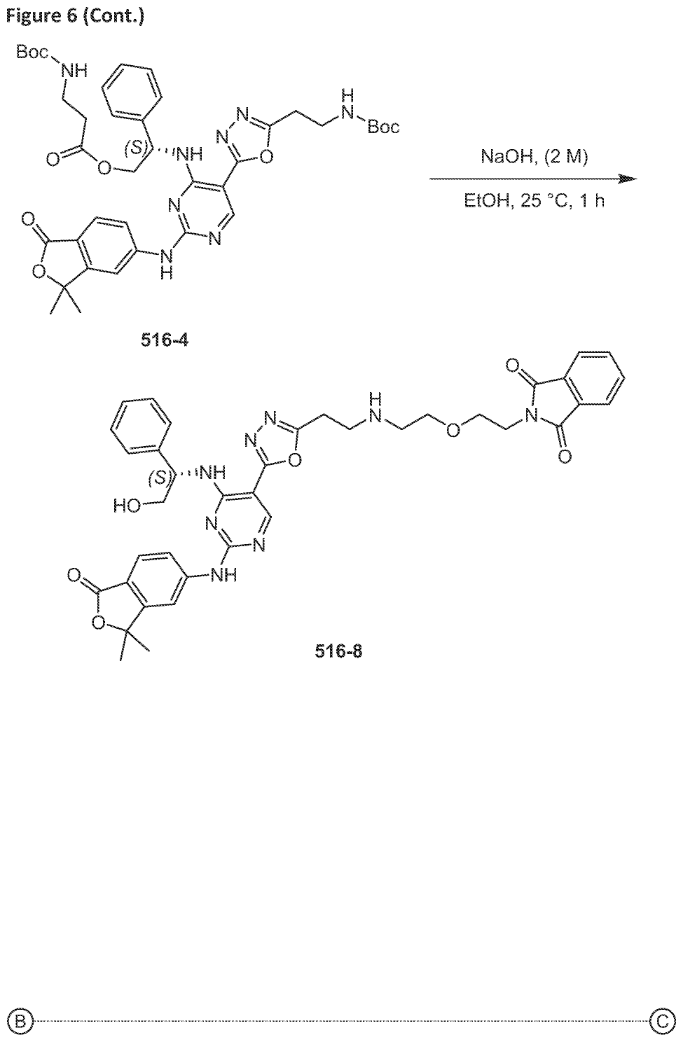

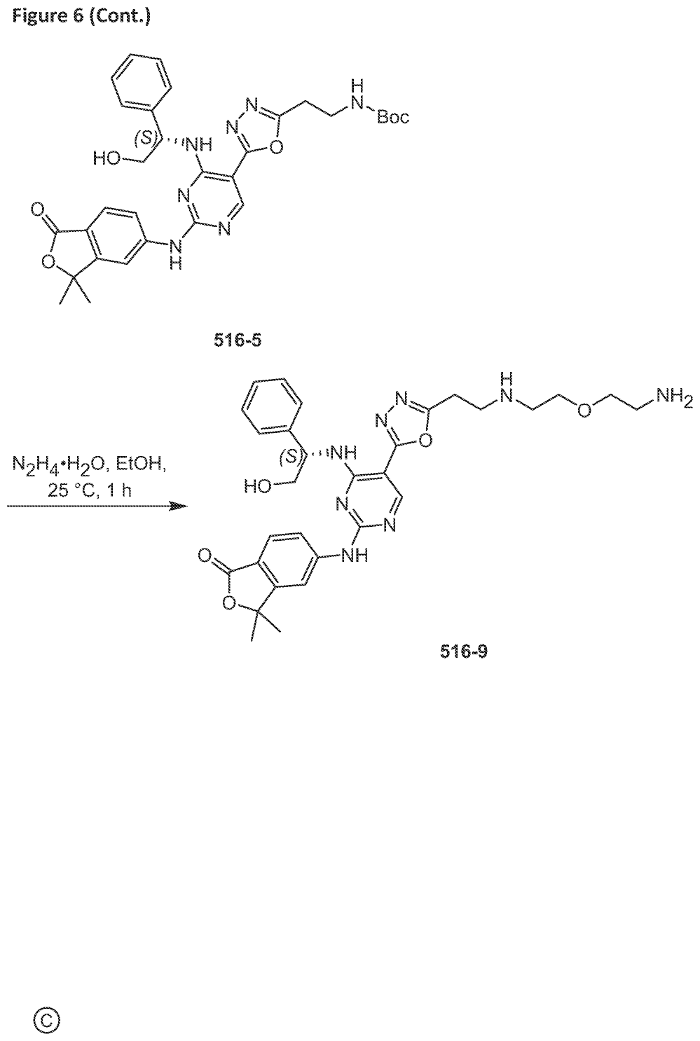

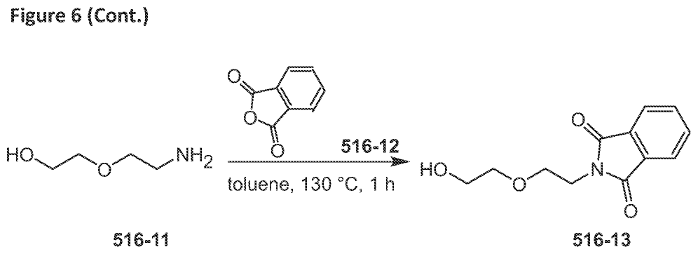

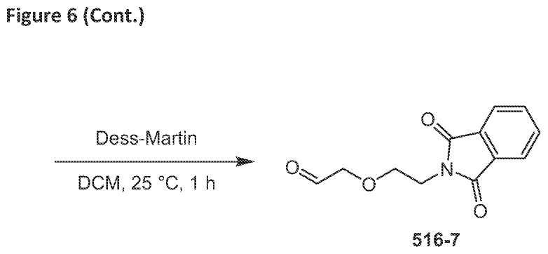

The reaction scheme for the synthesis of MS-5-016 is shown in FIG. 6.

The procedure for preparing 56-8 is described in the synthesis of MS-5-006.

Procedure for preparation of compound 516-13. To a solution of compound 516-10 (20 g, 190.23 mmol, 19.05 mL, 1 eq) and compound 516-11 (30.99 g, 209.25 mmol, 1.1 eq) in toluene (150 mL). The mixture was stirred at 130° C. for 1 h. The reaction mixture was concentrated under the reduced pressure to give compound 516-12 (49.51 g, crude) as a yellow oil. LCMS: t_{R}=0.278 min, MS (ESI+) m/z=235.8 [M+1]⁺. ¹H NMR (400 MHz, CDCl₃): δ=7.87 (dd, J=5.2, 2.8 Hz, 2H), 7.74 (dd, J=5.2, 2.8 Hz, 2H), 3.98-3.89 (m, 2H), 3.82-3.74 (m, 2H), 3.72-3.67 (m, 2H), 3.65-3.40 (m, 2H).

Procedure for preparation of compound 516-7. To a solution of compound 516-12 (1 g, 4.25 mmol, 1 eq) in DCM (15 mL) was added Dess-Martin (2.34 g, 5.53 mmol, 1.71 mL, 1.3 eq). The mixture was stirred at 25° C. for 1 h. To the mixture was added water (20 mL). The mixture was extracted with ethyl acetate (20 mL×3). The combined organic extracts were washed with brine (50 mL×3), dried, filtered and concentrated to give compound 516-7 (523 mg, crude) as a colourless oil. LCMS: t_{R}=0.258 min, MS (ESI+) m/z=233.8 [M+1]⁺. ¹H NMR (400 MHz, CDCl₃): δ=9.68 (s, 1H), 7.89 (dd, J=5.2, 2.8 Hz, 2H), 7.75 (dd, J=5.6, 2.8 Hz, 2H), 4.14 (s, 2H), 4.00-3.96 (m, 2H), 3.87-3.82 (m, 2H).

Procedure for preparation of compound 516-3. To a solution of compound 516-2 (1.27 g, 6.69 mmol, 1 eq) in DCM (30 mL) was added EDCI (1.28 g, 6.69 mmol, 1 eq), HOBt (903.89 mg, 6.69 mmol, 1 eq) and TEA (744.58 mg, 7.36 mmol, 1.02 mL, 1.1 eq) at 0° C. for 0.5 h. Then compound 56-8 (3 g, 6.69 mmol, 1 eq) was added, the mixture was stirred at 25° C. for 1.5 h. The reaction mixture was partitioned between DCM (50 mL) and water (50 mL). After quenching the reaction, the reaction mixture was poured into separatory funnel and separated. The residue was purified by prep-HPLC (FA condition) and lyophilized to give compound 516-3 (2.2 g, crude) as a yellow solid. LCMS (EC6212-103-P1A): t_{R}=0.476 min, MS (ESI+) m/z=791.3 [M+1]⁺.

Procedure for preparation of compound 516-4. To a solution of compound 516-3 (2.2 g, 2.78 mmol, 1 eq) in DCM (22 mL) was added TosCl (583.37 mg, 3.06 mmol, 1.1 eq) and TEA (422.23 mg, 4.17 mmol, 580.78 uL, 1.5 eq). The mixture was stirred at 25° C. for 1 h. The reaction mixture was partitioned between DCM (30 mL×2) and water (30 mL). After quenching the reaction, the reaction mixture was poured into separatory funnel and separated. The residue was purified by flash silica gel chromatography (SiO₂, DCM/MeOH=100/1 to 10/1) to give compound 516-4 (1.52 g, crude) as a yellow solid. LCMS: t_{R}=0.534 min, MS (ESI+) m/z=773.4 [M+1]⁺.

Procedure for preparation of compound 516-5. To a solution of compound 516-4 (1.5 g, 1.94 mmol, 1 eq) in EtOH (15 mL) was added NaOH (2 M, 1.94 mL, 2 eq). The mixture was stirred at 25° C. for 1 h. The reaction mixture was partitioned between EtOAc (30 mL×2) and water (30 mL). After quenching the reaction, the reaction mixture was poured into separatory funnel and separated. The residue was purified by flash silica gel chromatography (SiO₂, DCM/MeOH=100/1 to 10/1) to give compound 516-5 (800 mg, 1.33 mmol, 68.51% yield) as a yellow solid. LCMS (EC6212-141-P1B): t_{R}=0.462 min, MS (ESI+) m/z=602.1 [M+1]⁺.

Procedure for preparation of compound 516-6. To a solution of compound 516-5 (800 mg, 1.33 mmol, 1 eq) in DCM (8 mL) was added HCl/dioxane (4 M, 1.33 mL, 4 eq). The mixture was stirred at 25° C. for 1 h. The reaction mixture was filtered and the filter residue was concentrated under the reduced pressure to give compound 516-6 (647 mg, crude, HCl) as a yellow solid. LCMS: t_{R}=0.355 min, MS (ESI+) m/z=502.2 [M+1]⁺. ¹H NMR (400 MHz, DMSO-d₆): δ=10.58 (s, 1H), 9.05 (d, J=7.6 Hz, 1H), 8.75 (s, 1H), 8.36 (s, 3H), 7.95 (s, 1H), 7.74-7.61 (m, 2H), 7.42-7.34 (m, 4H), 7.30-7.23 (m, 1H), 5.49-5.39 (m, 1H), 5.36-5.19 (m, 1H), 3.97-3.89 (m, 1H), 3.87-3.81 (m, 1H), 3.41-3.24 (m, 4H), 1.59 (s, 3H), 1.54 (s, 3H).

Procedure for preparation of compound 516-8. To a solution of compound 516-6 (130 mg, 211.19 μmol, 1 eq, TFA) and compound 516-7 (49.25 mg, 211.19 μmol, 1 eq) in MeOH (3 mL), the mixture was stirred at 25° C. for 1 h. Then was added NaBH(OAc)₃ (134.28 mg, 633.57 μmol, 3 eq). The mixture was stirred at 25° C. for 1 h. The residue was purified by prep-HPLC (22%-52% ACN in water (0.1% TFA), 10 min) and lyophilized to give compound 516-8 (32 mg, 44.52 μmol, 21.08% yield) as a white solid. LCMS: t_{R}=0.389 min, MS (ESI+) m/z=719.0 [M+1]⁺. ¹H NMR (400 MHz, DMSO-d₆): δ=10.31 (s, 1H), 8.87 (d, J=8.0 Hz, 1H), 8.70-8.57 (m, 3H), 7.95 (s, 1H), 7.90-7.80 (m, 4H), 7.75-7.69 (m, 1H), 7.68-7.62 (m, 1H), 7.44-7.32 (m, 4H), 7.31-7.23 (m, 1H), 5.53-5.34 (m, 1H), 3.98-3.85 (m, 2H), 3.81 (t, J=5.6 Hz, 2H), 3.75-3.70 (m, 4H), 3.45 (d, J=5.6 Hz, 2H), 3.33 (d, J=6.8 Hz, 2H), 3.22 (d, J=4.4 Hz, 2H), 1.58 (s, 3H), 1.53 (s, 3H).

Procedure for preparation of compound 516-9. To a solution of compound 516-8 (27 mg, 37.56 μmol, 1 eq) in EtOH (1 mL) was added N₂H₄·H₂O (0.020 g, 399.52 μmol, 19.42 uL, 10.64 eq). The mixture was stirred at 25° C. for 1 h. The reaction mixture was concentrated under the reduced pressure to give compound 516-8 (38 mg, crude) as a white solid. LCMS: t_{R}=0.314 min, MS (ESI+) m/z=589.2 [M+1]⁺.

Procedure for preparation of MS-5-016. To a solution of compound 516-9 (38 mg, 64.55 μmol, 1 eq) and compound 57-15 (53.49 mg, 193.66 μmol, 3 eq) in DMAC (1 mL) was added DIEA (25.03 mg, 193.66 μmol, 33.73 uL, 3 eq). The mixture was stirred at 110° C. for 1 h. The residue was purified by prep-HPLC (18%-48% ACN in water (0.225% FA), 9 min) and prep-HPLC (26%-26% ACN in water (10 mM NH₄HCO₃), 2 min) and lyophilized to give MS-5-016 (2.06 mg, 2.19 μmol, 3.40% yield, 90% purity) as a yellow solid. LCMS: t_{R}=1.829 min, MS (ESI+) m/z=845.3 [M+1]⁺. ¹H NMR (400 MHz, DMSO-d₆): δ=11.08 (s, 1H), 10.26 (s, 1H), 8.87 (d, J=7.6 Hz, 1H), 8.62 (s, 1H), 7.97 (s, 1H), 7.73-7.67 (m, 1H), 7.66-7.61 (m, 1H), 7.56-7.51 (m, 1H), 7.42-7.33 (m, 4H), 7.29-7.23 (m, 1H), 7.10 (d, J=7.2 Hz, 1H), 7.00 (d, J=7.2 Hz, 1H), 6.61 (t, J=5.2 Hz, 1H), 5.45-5.37 (m, 1H), 5.23 (t, J=4.0 Hz, 1H), 5.09-4.99 (m, 1H), 4.15-3.98 (m, 1H), 3.91-3.81 (m, 2H), 3.61 (t, J=4.8 Hz, 2H), 3.53-3.50 (m, 2H), 3.46 (d, J=6.0 Hz, 2H), 3.03 (dd, J=12.4, 5.2 Hz, 4H), 2.91-2.83 (m, 1H), 2.73 (t, J=5.2 Hz, 2H), 2.59 (dd, J=4.0, 1.2 Hz, 2H), 2.06-1.99 (m, 1H), 1.58 (s, 3H), 1.53 (s, 3H).

### Example 9

### Synthesis of MS-5-017

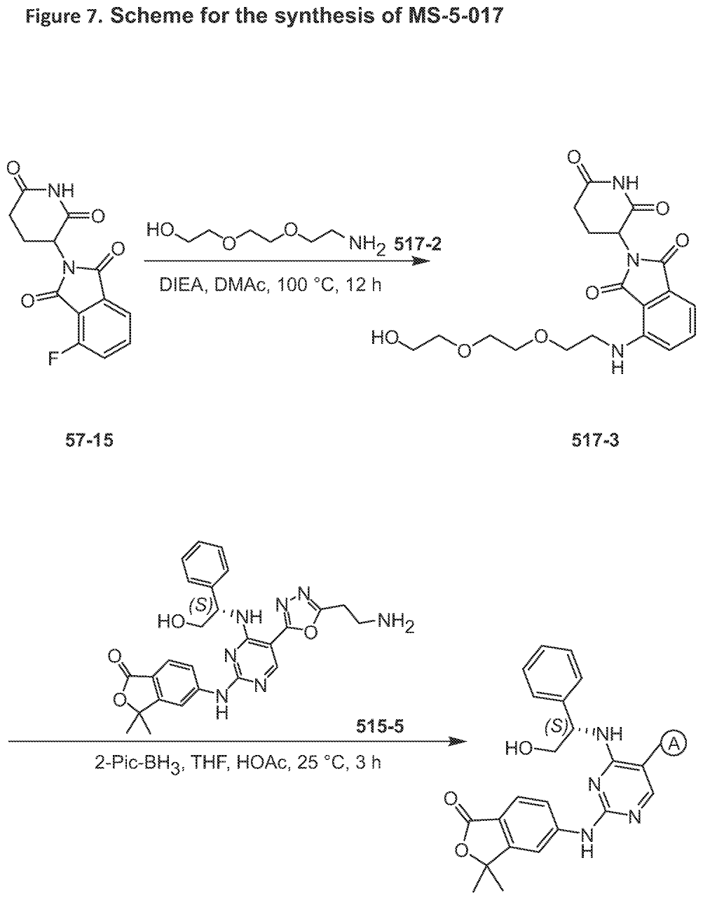

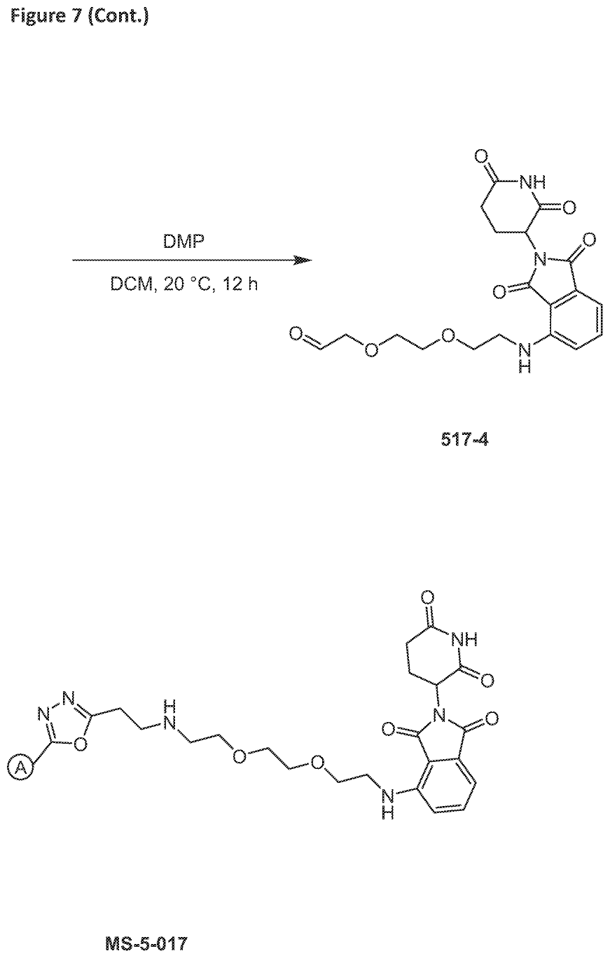

The reaction scheme for the synthesis of MS-5-017 is shown in FIG. 7.

The procedure for preparing 515-5 is described in the synthesis of MS-5-015.

Procedure for preparation of compound 517-3. To a solution of compound 57-15 (2 g, 7.24 mmol, 1 eq) and compound 517-2 (1.62 g, 10.86 mmol, 1.5 eq) in DMAc (10 mL) was added DIEA (2.81 g, 21.72 mmol, 3.78 mL, 3 eq), the reaction mixture was stirred at 100° C. for 12 h. The reaction mixture was quenched by addition H₂O (10 mL) and extracted with EtOAc (100×4 mL), the combined organic layers was wished with brine (30 mL) and concentrated under reduced pressure to give a residue. The residue was purified by flash silica gel chromatography (SiO₂, PE/EA=50/1 to 0/1) to give compound 517-3 (1.04 g, 2.57 mmol, 35.43% yield) as a green oil. LCMS: t_{R}=0.332 min, MS (ESI+) m/z=405.9 [M+1]⁺. ¹H NMR (400 MHz, DMSO-d₆): δ=11.10 (s, 1H), 7.59 (dd, J=8.4, 7.2 Hz, 1H), 7.15 (d, J=8.4 Hz, 1H), 7.05 (d, J=6.8 Hz, 1H), 6.61 (t, J=6.0 Hz, 1H), 5.06 (dd, J=12.8, 5.2 Hz, 1H), 5.02-5.02 (m, 1H), 4.56 (t, J=5.2 Hz, 1H), 3.65-3.61 (m, 2H), 3.58 (s, 4H), 3.51-3.46 (m, 4H), 3.44-3.41 (m, 2H), 2.91-2.81 (m, 1H), 2.64-2.53 (m, 2H), 2.07-2.01 (m, 1H).

Procedure for preparation of compound 517-4. To a solution of compound 517-3 (300 mg, 740.01 μmol, 1 eq) in DCM (5 mL) was added DMP (376.64 mg, 888.01 μmol, 274.92 uL, 1.2 eq), the reaction mixture was stirred at 20° C. for 12 h. The reaction mixture was quenched by addition NaHCO₃ (aq.) (40 mL) and extracted with DCM (40×3 mL), the combined organic layers was washed with brine (40 mL) and concentrated under reduced pressure to give a residue. The residue was purified by prep-TLC (SiO₂, PE/EA=50/1 to 0:1) to give compound 517-4 (339 mg, 840.39 μmol, 56.78% yield) as a yellow solid. LCMS: t_{R}=0.304 min, MS (ESI+) m/z=404.0 [M+1]⁺, H NMR (400 MHz, DMSO-d₆): δ=11.10 (s, 1H), 9.61-9.51 (m, 1H), 7.63-7.51 (m, 1H), 7.22-7.08 (m, 1H), 6.61 (s, 1H), 5.83-5.66 (m, 1H), 5.06 (d, J=8.4 Hz, 1H), 3.73-3.38 (m, 10H), 2.97-2.80 (m, 1H), 2.59 (d, J=17.2 Hz, 2H), 2.10-1.96 (m, 1H).

Procedure for preparation of MS-5-017. To a solution of compound 517-4 (66.79 mg, 165.57 μmol, 8.91e-1 eq) and compound 515-5 (100 mg, 185.87 μmol, 1 eq, HCl) in THF (1 mL) was added HOAc (33.49 mg, 557.61 μmol, 31.89 uL, 3 eq) and borane;2-methylpyridine (39.76 mg, 371.74 μmol, 2 eq). The mixture was stirred at 25° C. for 3 h. The residue was purified by prep-HPLC (52%-82% ACN in water (10 mM NH₄HCO₃), 9 min) and prep-TLC (SiO₂, DCM/MeOH=10:1) and eluted with ACN (10 mL) to give MS-5-017 (5.59 mg, 6.21 μmol, 3.34% yield, 98.8% purity) as a yellow solid. LCMS: t_{R}=0.400 min, MS (ESI+) m/z=889.4 [M+1]⁺. ¹H NMR (400 MHz, CDCl₃): δ=8.80-8.70 (m, 1H), 8.26 (s, 1H), 8.12 (d, J=7.2 Hz, 1H), 8.02 (s, 1H), 7.96-7.88 (m, 1H), 7.61 (dd, J=8.4, 7.2 Hz, 1H), 7.42 (d, J=6.8 Hz, 1H), 7.21 (d, J=8.4 Hz, 1H), 6.17 (s, 1H), 5.23 (s, 2H), 4.98 (dd, J=12.0, 5.2 Hz, 1H), 4.65 (q, J=7.2 Hz, 2H), 4.25-4.14 (m, 1H), 3.88-3.80 (m, 2H), 3.66 (d, J=2.4 Hz, 2H), 3.48-3.32 (m, 5H), 2.94-2.88 (m, 1H), 2.86-2.79 (m, 1H), 2.79-2.72 (m, 1H), 2.23 (s, 1H), 2.11 (dd, J=11.2, 5.2 Hz, 2H), 2.04-2.01 (m, 2H), 1.98 (d, J=7.2 Hz, 2H), 1.90 (t, J=5.2 Hz, 4H), 1.75-1.71 (m, 1H), 1.26 (s, 3H), 0.89 (t, J=6.4 Hz, 3H).

### Example 10

### Synthesis of MS-5-018

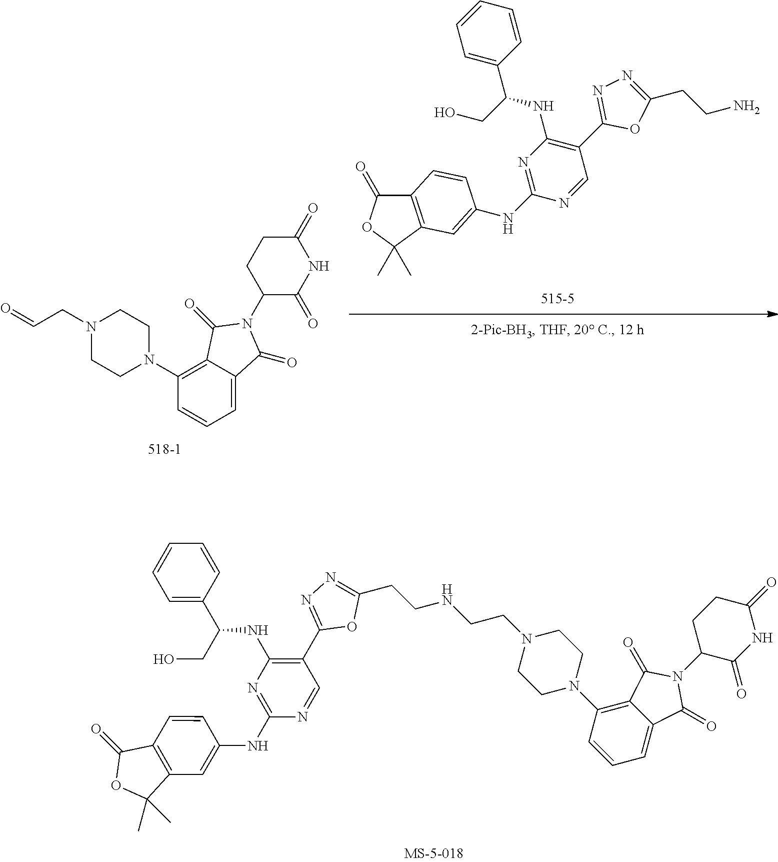 [SMILES: O=CCN1CCN(c2cccc3c2C(=O)N(C2CCC(=O)NC2=O)C3=O)CC1 ; CC1(C)OC(=O)c2ccc(Nc3ncc(-c4nnc(CCN)o4)c(N[C@H](CO)c4ccccc4)n3)cc21 ; CC1(C)OC(=O)c2ccc(Nc3ncc(-c4nnc(CCNCCN5CCN(c6cccc7c6C(=O)N(C6CCC(=O)NC6=O)C7=O)CC5)o4)c(N[C@H](CO)c4ccccc4)n3)cc21]

The procedure for preparing 515-5 is described in the synthesis of MS-5-015.

Procedure for preparation of compound MS-5-018. To a solution of compound 518-1 (80 mg, 208.12 μmol, 1 eq) and compound 515-5 (104.38 mg, 208.12 μmol, 1 eq) in DMAc (1 mL) was added 2-Pic-BH₃ (22.26 mg, 208.12 μmol, 1 eq) and the mixture was stirred at 25° C. for 2 h. The reaction mixture was filtered and purified by prep-HPLC (25%-55% ACN in water (10 mM NH₄HCO₃), 8 min) to give compound MS-5-018 (7.50 mg, 8.41 μmol, 4.04% yield, 97.58% purity) as a yellow solid. LCMS: t_{R}=0.379 min, MS (ESI+) m/z=870.5[M+1]⁺. ¹H NMR (400 MHz, DMSO-d₆): δ=11.27-10.90 (m, 1H), 10.41-10.16 (m, 1H), 8.90 (d, J=8.0 Hz, 1H), 8.67 (s, 1H), 7.96 (s, 1H), 7.74-7.68 (m, 1H), 7.67-7.62 (m, 1H), 7.61-7.54 (m, 1H), 7.42-7.38 (m, 2H), 7.37-7.33 (m, 2H), 7.31-7.18 (m, 3H), 5.46-5.38 (m, 1H), 5.24 (t, J=4.8 Hz, 1H), 5.08 (dd, J=5.2, 12.8 Hz, 1H), 3.95-3.81 (m, 2H), 3.24 (s, 4H), 3.14-3.00 (m, 4H), 2.92-2.82 (m, 1H), 2.71 (t, J=6.0 Hz, 2H), 2.61-2.54 (m, 6H), 2.48-2.43 (m, 2H), 2.07-1.96 (m, 1H), 1.60-1.52 (m, 6H).

### Example 11

### Synthesis of MS-5-019

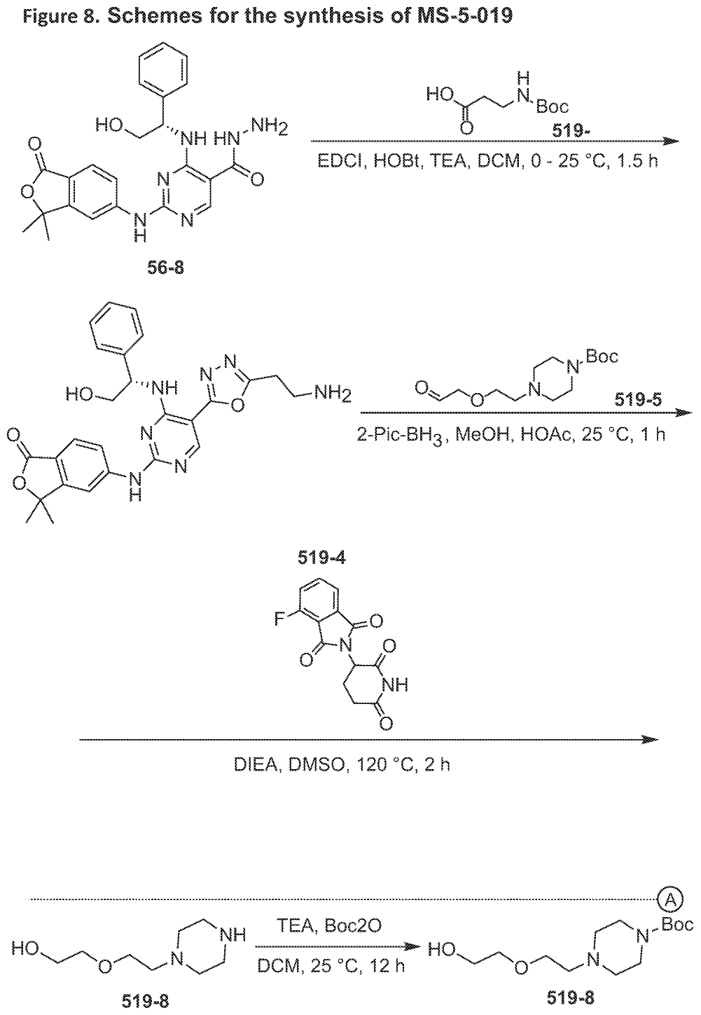

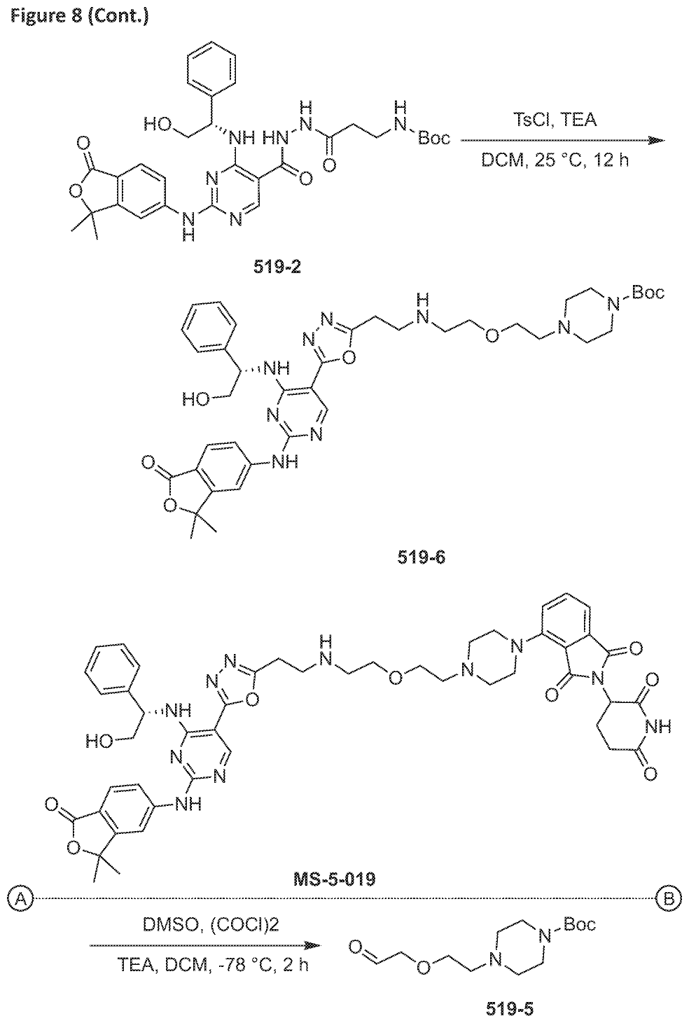

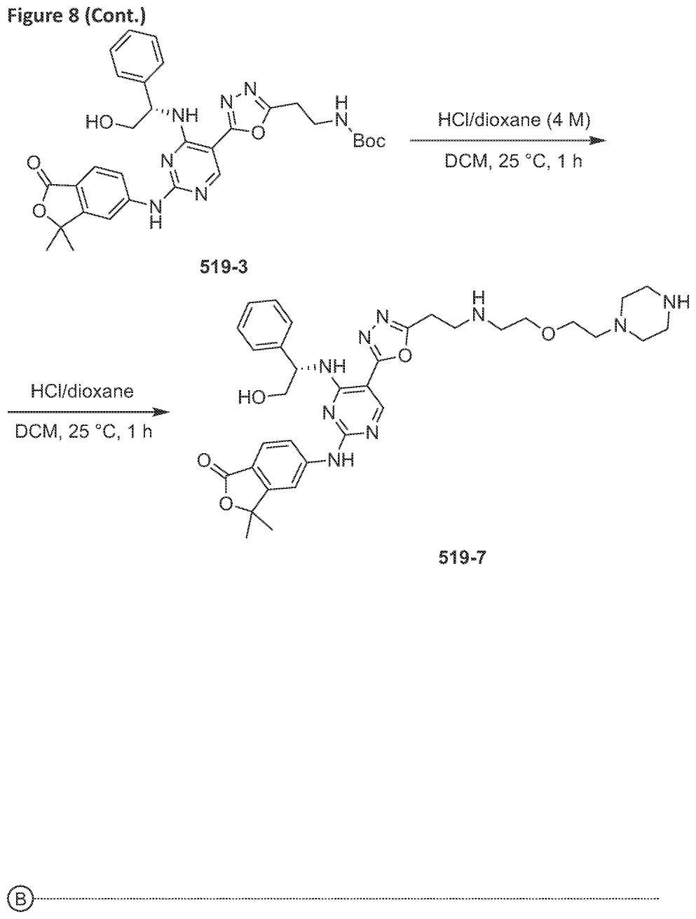

The reaction scheme for the synthesis of MS-5-019 is shown in FIG. 8.

The procedure for preparing 56-8 is described in the synthesis of MS-5-006.

Procedure for preparation of compound 519-2. To a solution of compound 56-8 (2.78 g, 14.72 mmol, 1.2 eq) in DCM (55 mL) was added EDCI (3.53 g, 18.40 mmol, 1.5 eq), HOBt (1.66 g, 12.26 mmol, 1 eq) and TEA (3.72 g, 36.79 mmol, 5.12 mL, 3 eq) at 0° C. for 0.5 h. Then compound 519-1 (5.5 g, 12.26 mmol, 1 eq) was added, the mixture was stirred at 25° C. for 1 h. The reaction mixture was partitioned between DCM (50 mL) and water (100 mL). After quenching the reaction, the reaction mixture was poured into separatory funnel and separated. The residue was purified by flash silica gel chromatography (SiO₂, DCM/MeOH=100/1 to 10/1) to give compound 519-2 (3.71 g, 5.99 mmol, 48.82% yield) as a white solid. LCMS: t_{R}=0.406 min, MS (ESI+) m/z=620.0 [M+1]⁺. ¹H NMR (400 MHz, DMSO-d₆) δ=10.23 (s, 1H), 10.16 (s, 1H), 9.86 (s, 1H), 9.53 (d, J=8.0 Hz, 1H), 8.67 (s, 1H), 7.96 (s, 1H), 7.72-7.66 (m, 1H), 7.65-7.56 (m, 1H), 7.36 (d, J=4.4 Hz, 4H), 7.29-7.18 (m, 1H), 6.83 (t, J=5.2 Hz, 1H), 5.39-5.23 (m, 1H), 5.16 (t, J=4.8 Hz, 1H), 3.90-3.79 (m, 1H), 3.78-3.69 (m, 1H), 3.19 (m, 2H), 2.37 (t, J=7.2 Hz, 2H), 1.55 (d, J=18.8 Hz, 6H), 1.39 (s, 9H)

Procedure for preparation of compound 519-3. To a solution of compound 519-2 (3.71 g, 5.99 mmol, 1 eq) in DCM (40 mL) was added TosCl (1.26 g, 6.59 mmol, 1.1 eq) and TEA (908.74 mg, 8.98 mmol, 1.25 mL, 1.5 eq). The mixture was stirred at 25° C. for 12 h. The reaction mixture was partitioned between DCM (30 mL) and water (30 mL). After quenching the reaction, the reaction mixture was poured into separatory funnel and separated. The residue was purified by flash silica gel chromatography (SiO₂, DCM/MeOH=100/1 to 10/1) to give compound 519-3 (3.11 g, 5.17 mmol, 86.34% yield) as a colourless solid. LCMS: t_{R}=0.456 min, MS (ESI+) m/z=602.3 [M+1]⁺. ¹H NMR (400 MHz, DMSO-d₆) δ=10.31 (s, 1H), 8.88 (d, J=7.6 Hz, 1H), 8.64 (s, 1H), 7.98 (s, 1H), 7.75-7.68 (m, 1H), 7.67-7.61 (m, 1H), 7.45-7.33 (m, 4H), 7.30-7.23 (m, 1H), 7.08 (t, J=5.6 Hz, 1H), 5.50-5.36 (m, 1H), 5.24 (t, J=4.8 Hz, 1H), 3.99-3.88 (m, 1H), 3.87-3.77 (m, 1H), 3.39 (q, J=5.6 Hz, 2H), 3.05 (t, J=6.4 Hz, 2H), 1.58 (s, 3H), 1.53 (s, 3H), 1.36 (s, 9H).

Procedure for preparation of compound 519-4. To a solution of compound 519-3 (500 mg, 831.05 μmol, 1 eq) in DCM (5 mL) was added HCl/dioxane (4 M, 623.28 uL, 3 eq). The mixture was stirred at 25° C. for 1 h. The reaction mixture was filtered and the filter residue was concentrated under the reduced pressure to give 519-4 (460 mg, 641.27 μmol, 77.16% yield, 75% purity, HCl) as a white solid. LCMS: t_{R}=0.338 min, MS (ESI+) m/z=502.1 [M+1]⁺ Procedure for preparation of compound 519-9. To a solution of compound 519-8 (5 g, 28.70 mmol, 4.72 mL, 1 eq) in DCM (30 mL) was added TEA (5.81 g, 57.39 mmol, 7.99 mL, 2 eq) and Boc₂O (6.89 g, 31.57 mmol, 7.25 mL, 1.1 eq) and the mixture was stirred at 25° C. for 12 h. The reaction mixture was concentrated under reduced pressure to give a residue. The residue was purified by flash silica gel chromatography (SiO₂, PE/EA=50/1 to 0/1) to give compound 519-9 (6 g, 21.87 mmol, 76.21% yield) as a yellow oil. LCMS: t_{R}=0.208 min, MS (ESI+) m/z=275.2 [M+1]⁺. ¹H NMR (400 MHz, CDCl₃): δ=3.65-3.52 (m, 6H), 3.42-3.36 (m, 4H), 2.56-2.50 (m, 2H), 2.45-2.36 (m, 4H), 1.39 (s, 9H).

Procedure for preparation of compound 519-5. To a solution of DMSO (1.42 g, 18.22 mmol, 1.42 mL, 5 eq) in DCM (20 mL) was added a mixture of (COCl)₂ (925.27 mg, 7.29 mmol, 638.11 uL, 2 eq) in DCM (20 mL) and stirred at −78° C. for 30 min and then compound 519-9 (1 g, 3.64 mmol, 1 eq) in DCM (20 mL) was added, then TEA (2.95 g, 29.16 mmol, 4.06 mL, 8 eq) was added and the resulting mixture was stirred at −78° C. for another 1.5 h. The reaction mixture was concentrated under the reduced pressure to give compound 519-5 (896 mg, crude) as a yellow oil. ¹H NMR (400 MHz, CDCl₃): δ=9.72 (s, 1H), 4.13 (s, 2H), 3.71-3.68 (m, 2H), 3.44 (d, J=5.2 Hz, 4H), 2.47 (d, J=4.8 Hz, 6H), 1.46 (s, 9H).

Procedure for preparation of compound 519-6. To a solution of compound 519-4 (460 mg, 641.27 μmol, 75% purity, 1 eq, HCl) and compound 519-5 (261.96 mg, 961.90 μmol, 1.5 eq) in MeOH (2 mL) and HOAc (0.2 mL) was added borane; 2-methylpyridine (68.59 mg, 641.27 μmol, 1 eq). The mixture was stirred at 25° C. for 1 h. The reaction mixture was concentrated under reduced pressure to give a residue. The residue was purified by prep-HPLC (15%-45% ACN in water (0.05% HCl), 20 min) and lyophilized to give compound 519-6 (196 mg, 258.62 μmol, 40.33% yield) as a white solid. LCMS: t_{R}=0.351 min, MS (ESI+) m/z=758.0 [M+1]⁺. ¹H NMR (400 MHz, DMSO-d₆): δ=10.29 (s, 1H), 8.88 (d, J=7.6 Hz, 1H), 8.65 (s, 1H), 7.98 (s, 1H), 7.75-7.68 (m, 1H), 7.66-7.60 (m, 1H), 7.46-7.32 (m, 4H), 7.30-7.22 (m, 1H), 5.48-5.37 (m, 1H), 5.23 (s, 1H), 3.97-3.88 (m, 1H), 3.87-3.77 (m, 1H), 3.47 (m, 4H), 3.28-3.24 (m, 4H), 3.07 (dd, J=14.0, 5.2 Hz, 4H), 2.74 (t, J=4.8 Hz, 2H), 2.47-2.43 (m, 2H), 2.33 (t, J=4.4 Hz, 4H), 1.58 (s, 3H), 1.53 (s, 3H), 1.36 (s, 9H).

Procedure for preparation of compound 519-7. To a solution of compound 519-6 (196 mg, 258.62 μmol, 1 eq) in DCM (2 mL) was added HCl/dioxane (4 M, 193.96 uL, 3 eq). The mixture was stirred at 25° C. for 1 h. The reaction mixture was filtered and the filter residue was concentrated under the reduced pressure to give compound 519-7 (226 mg, crude, HCl) as a white solid. LCMS: t_{R}=0.319 min, MS (ESI+) m/z=658.2 [M+1]⁺ Procedure for preparation of MS-5-019. To a solution of compound 519-7 (30 mg, 45.61 μmol, 1 eq) and 2-(2,6-dioxopiperidin-3-yl)-4-fluoroisoindoline-1,3-dione (10.71 mg, 38.77 μmol, 0.85 eq) in DMSO (0.5 mL) was added DIEA (8.84 mg, 68.41 μmol, 11.92 uL, 1.5 eq). The mixture was stirred at 120° C. for 2 h. The residue was purified by prep-HPLC (26%-56% ACN in water (10 mM NH₄HCO₃), 8 min) and lyophilized to give MS-5-019 (3.87 mg, 4.11 μmol, 9.01% yield, 97% purity) as a yellow solid. LCMS: t_{R}=1.145 min, MS (ESI+) m/z=914.3 [M+1]⁺. ¹H NMR (400 MHz, DMSO-d₆): δ=11.08 (s, 1H), 10.28 (s, 1H), 8.94-8.81 (m, 1H), 8.65 (s, 1H), 7.96 (s, 1H), 7.76-7.68 (m, 1H), 7.67-7.59 (m, 2H), 7.44-7.20 (m, 7H), 5.50-5.35 (m, 1H), 5.23 (s, 1H), 5.08 (dd, J=13.2, 5.2 Hz, 1H), 3.96-3.88 (m, 1H), 3.87-3.78 (m, 1H), 3.58-3.51 (m, 4H), 3.26 (s, 4H), 3.15 (s, 4H), 2.94-2.78 (m, 3H), 2.62-2.53 (m, 8H), 2.04-1.98 (m, 1H), 1.57 (s, 3H), 1.52 (s, 3H).

### Example 12

### Synthesis of MS-5-021

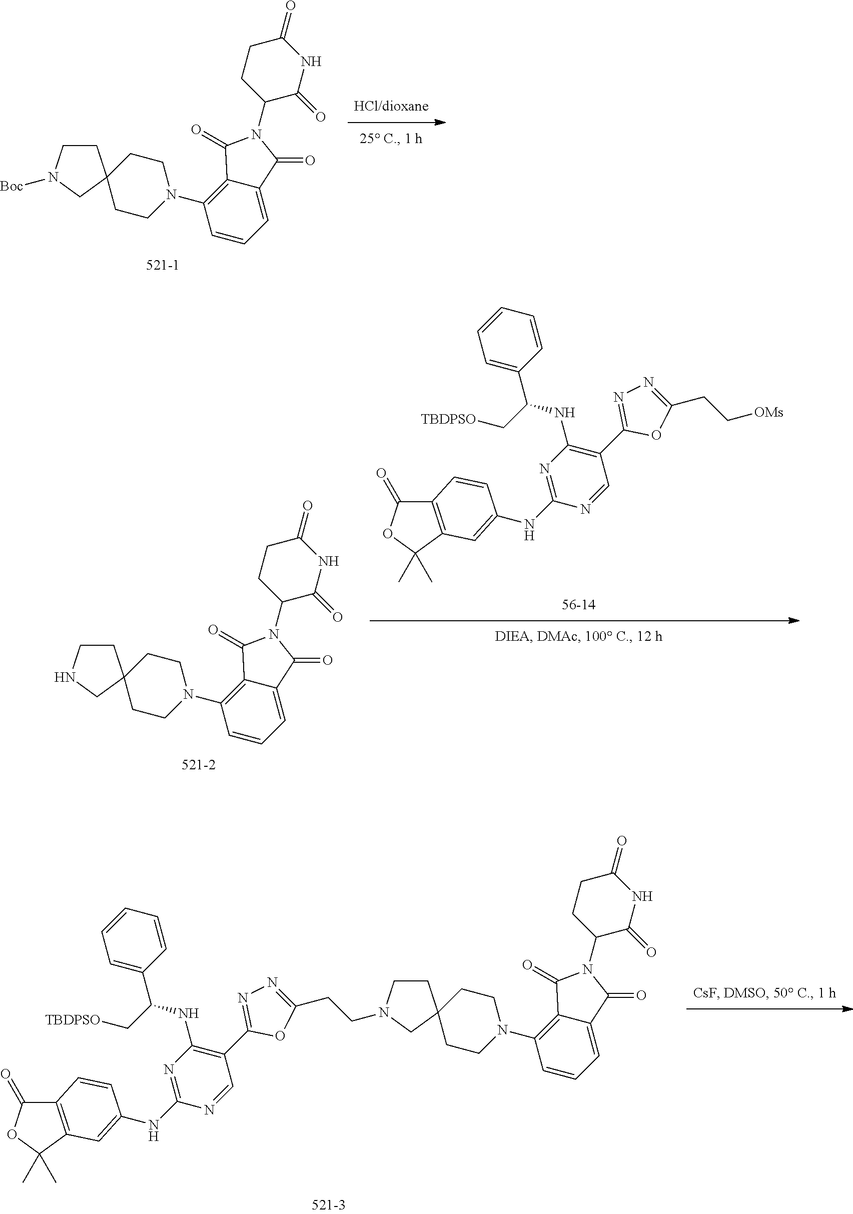

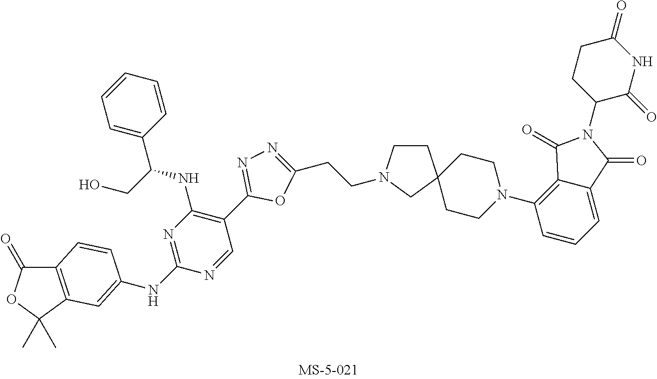 [SMILES: CC1(C)OC(=O)c2ccc(Nc3ncc(-c4nnc(CCN5CCC6(CCN(c7cccc8c7C(=O)N(C7CCC(=O)NC7=O)C8=O)CC6)C5)o4)c(N[C@H](CO)c4ccccc4)n3)cc21]

The procedure for preparing compound 521-1 is described in the synthesis of MS-6-019.

The procedure for preparing 56-14 is described in the synthesis of MS-5-006.

Procedure for preparation of compound 521-2. To a solution of compound 521-1 (1 g, 2.01 mmol, 1 eq) in DCM (10 mL) was added HCl/dioxane (4 M, 1.51 mL, 3 eq). The mixture was stirred at 25° C. for 1 h. The reaction mixture was filtered and the filter residue was concentrated under the reduced pressure to give compound 521-2 (677 mg, 1.56 mmol, 77.65% yield, HCl) as a yellow solid. LCMS: t_{R}=0.262 m, MS (ESI+) m/z=397.0[M+1]H Procedure for preparation of compound 521-3. To a solution of compound 521-2 (300.00 mg, 693.00 μmol, 1 eq, HCl) and compound 56-14 (567.57 mg, 693.00 μmol, 1 eq) in DMAc (5 mL) was added DIEA (358.26 mg, 2.77 mmol, 482.83 uL, 4 eq). The mixture was stirred at 100° C. for 12 h. The residue was purified by prep-PLC (410-71%0 ACN in water (0.225% FA), 10 min) and lyophilized to give compound 521-3 (123 mg, 109.89 μmol, 15.86% yield) as a yellow solid. LCMS: t_{R}=0.526 min, MS (ESI+) m/z=1119.9 [M+1]⁺. ¹H NMR (400 MHz, DMSO-d₆): δ=11.09 (s, 1H), 10.50-10.15 (m, 1H), 9.25 (d, J=8.0 Hz, 1H), 8.71 (s, 1H), 7.87 (s, 1H), 7.78-7.49 (m, 5H), 7.48-7.19 (m, 15H), 5.64-5.50 (m, 1H), 5.09 (dd, J=12.8, 4.4 Hz, 1H), 4.08-3.94 (m, 2H), 3.55 (s, 1H), 3.68-3.50 (m, 1H), 3.27 (d, J=9.6 Hz, 6H), 2.90-2.83 (m, 1H), 2.64-2.56 (m, 2H), 1.96 (d, J=2.0 Hz, 1H), 1.81-1.63 (m, 4H), 1.52 (d, J=7.6 Hz, 6H), 1.40 (s, 2H), 1.17 (t, J=7.2 Hz, 2H), 0.92 (s, 9H).

Procedure for preparation of MS-5-021. To a solution of compound 521-3 (100 mg, 89.34 μmol, 1 eq) in DMSO (2 mL) was added CsF (135.71 mg, 893.38 μmol, 32.94 uL, 10 eq). The mixture was stirred at 50° C. for 1 h. The residue was purified by prep-HPLC (33%-63% ACN in water (0.225% FA), 10 min) and lyophilized to give MS-5-021 (22.89 mg, 24.27 μmol, 27.16% yield, 98.27% purity, FA) as a yellow solid. LCMS: t_{R}=1.330 min, MS (ESI+) m/z=881.5 [M+1]⁺. ¹H NMR (400 MHz, DMSO-d₆): δ=11.08 (s, 1H), 10.30 (s, 1H), 8.88 (d, J=7.6 Hz, 1H), 8.66 (s, 1H), 8.24 (s, 1H), 7.97 (s, 1H), 7.76-7.59 (m, 3H), 7.46-7.30 (m, 6H), 7.29-7.22 (m, 1H), 5.52-5.37 (m, 1H), 5.33-5.17 (m, 1H), 5.08 (dd, J=12.8, 5.2 Hz, 1H), 4.02-3.88 (m, 1H), 3.87-3.76 (m, 1H), 3.23 (s, 6H), 3.13 (t, J=7.2 Hz, 2H), 2.94-2.82 (m, 3H), 2.65-2.53 (m, 4H), 2.06-1.94 (m, 1H), 1.73-1.60 (m, 6H), 1.58 (s, 3H), 1.53 (s, 3H).

### Example 13

### Synthesis of MS-5-022

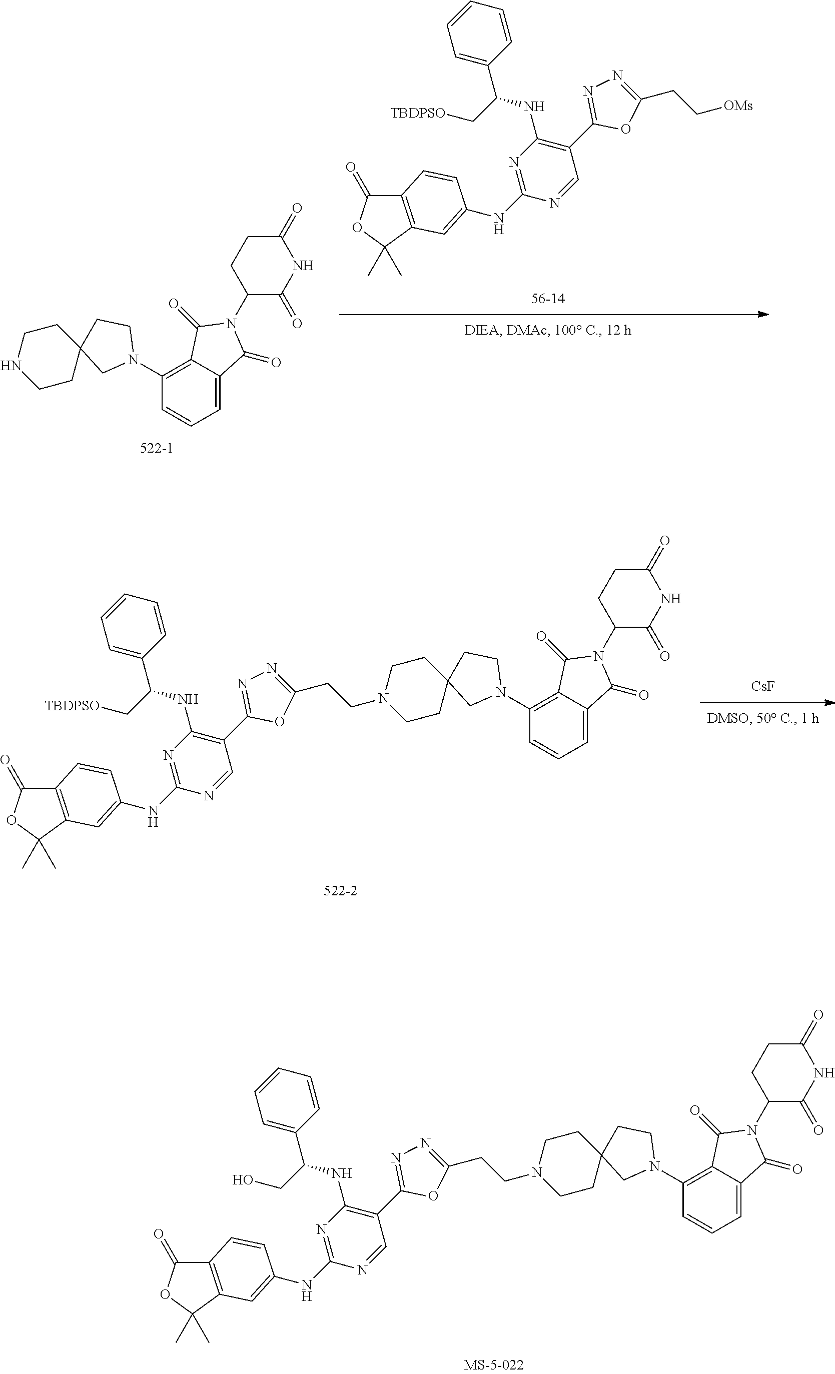

The procedure for preparing compound 522-1 is described in the synthesis of MS-6-031.

The procedure for preparing 56-14 is described in the synthesis of MS-5-006.

Procedure for preparation of compound 522-2. To a solution of compound 522-1 (280 mg, 646.80 μmol, 1 eq, HCl) and compound 56-14 (529.74 mg, 646.80 μmol, 1 eq) in DMAc (5 mL) was added DIEA (334.38 mg, 2.59 mmol, 450.64 uL, 4 eq). The mixture was stirred at 100° C. for 12 h. The residue was purified by prep-HPLC (41%-71% ACN in water (0.225% FA), 10 min) and lyophilized to give compound 522-2 (151 mg, 134.90 μmol, 20.86% yield) as a yellow solid. LCMS: t_{R}=0.528 min, MS (ESI+) m/z=1119.4 [M+1]⁺. ¹H NMR (400 MHz, DMSO-d₆): δ=11.06 (s, 1H), 10.31 (s, 1H), 9.23 (d, J=8.4 Hz, 1H), 8.69 (s, 1H), 7.88 (s, 1H), 7.73 (d, J=8.4 Hz, 1H), 7.65-7.48 (m, 4H), 7.46-7.02 (m, 15H), 5.65-5.51 (m, 1H), 5.06 (dd, J=12.0, 5.2 Hz, 1H), 4.20-4.10 (m, 1H), 4.00-3.94 (m, 1H), 3.68-3.55 (m, 2H), 3.52-3.39 (m, 4H), 3.23-3.06 (m, 2H), 2.88-2.82 (m, 1H), 2.66-2.52 (m, 4H), 1.95 (s, 1H), 1.81 (dd, J=4.0, 2.8 Hz, 2H), 1.52 (d, J=5.6 Hz, 9H), 1.17 (t, J=7.2 Hz, 2H), 0.91 (s, 9H).

Procedure for preparation of MS-5-022. To a solution of compound 522-3 (100 mg, 89.34 μmol, 1 eq) in DMSO (0.5 mL) was added CsF (135.71 mg, 893.38 μmol, 32.94 uL, 10 eq). The mixture was stirred at 50° C. for 1 h. The residue was purified by prep-HPLC (33%-63% ACN in water (0.05% HCl), 10 min) and lyophilized to give MS-5-022 (22.64 mg, 25.15 μmol, 28.16% yield, 97.88% purity) as a yellow solid. LCMS: t_{R}=1.346 min, MS (ESI+) m/z=881.3 [M+1]⁺. ¹H NMR (400 MHz, DMSO-d₆): δ=11.06 (s, 1H), 10.29 (s, 1H), 8.88 (d, J=7.6 Hz, 1H), 8.65 (s, 1H), 8.19 (s, 1H), 7.98 (s, 1H), 7.76-7.68 (m, 1H), 7.66-7.61 (m, 1H), 7.59-7.52 (m, 1H), 7.46-7.32 (m, 4H), 7.31-7.23 (m, 1H), 7.18-7.04 (m, 2H), 5.55-5.38 (m, 1H), 5.36-5.15 (m, 1H), 5.06 (dd, J=12.8, 5.2 Hz, 1H), 4.05-3.88 (m, 1H), 3.88-3.78 (m, 1H), 3.61 (t, J=6.0 Hz, 2H), 3.46-3.42 (m, 2H), 3.13 (t, J=7.2 Hz, 2H), 2.93-2.78 (m, 3H), 2.63-2.53 (m, 4H), 2.47-2.36 (m, 2H), 2.05-1.94 (m, 1H), 1.81 (t, J=6.4 Hz, 2H), 1.55 (d, J=19.6 Hz, 10H).

### Example 14

### Synthesis of MS-5-024

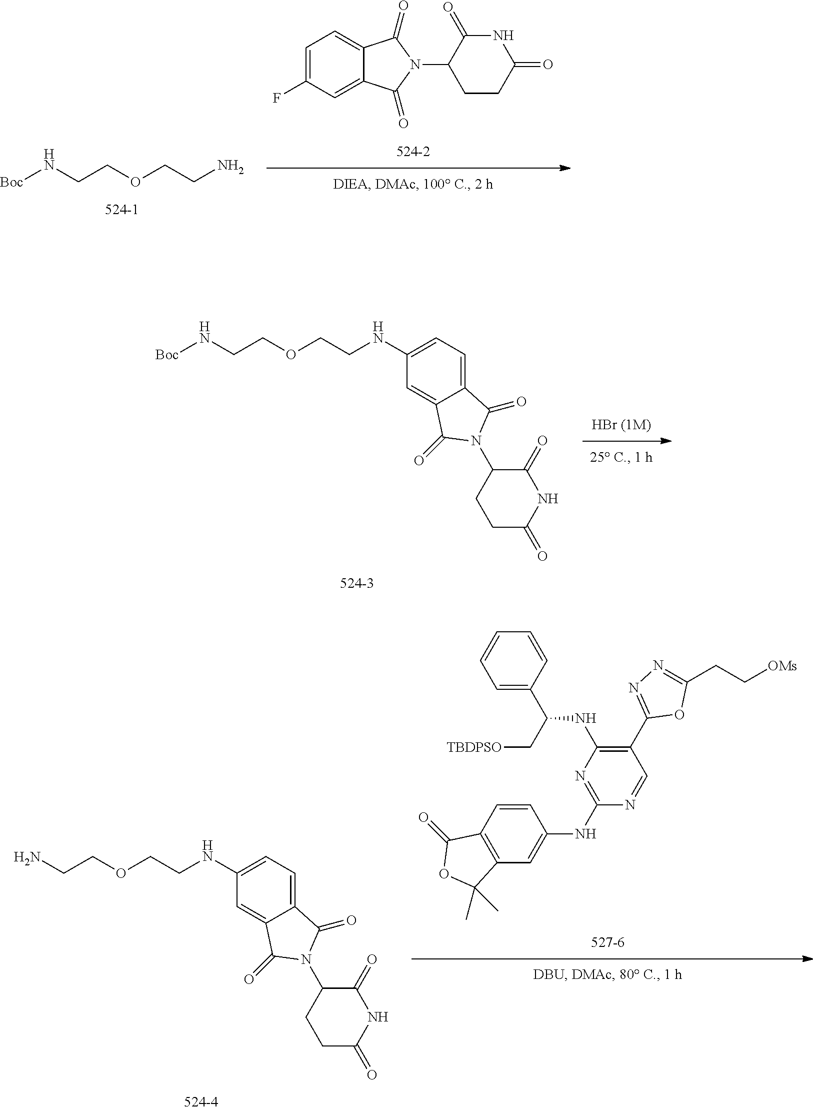

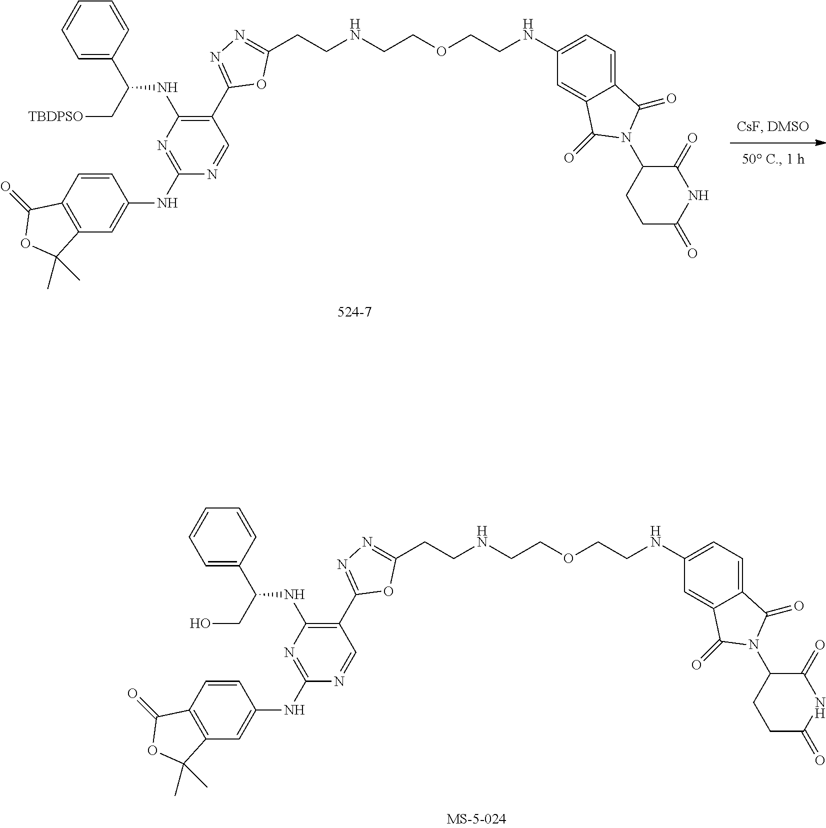

The procedure for preparing compound 527-6 is described in the synthesis of MS-5-027.

Procedure for preparation of compound 524-3. To a solution of compound 524-1 (commercially available, 500 mg, 2.45 mmol, 1 eq) and compound 524-2 (commercially available, 676.13 mg, 2.45 mmol, 1 eq) in DMAc (5 mL) was added DIEA (632.72 mg, 4.90 mmol, 852.72 uL, 2 eq). The mixture was stirred at 100° C. for 2 h. The residue was purified by prep-HPLC (26%-56% ACN in water (0.225% FA), 10 min) and lyophilized to give compound 524-3 (112 mg, 243.22 μmol, 9.94% yield) as a yellow solid. LCMS: t_{R}=0.394 min, MS (ESI+) m/z=361.0 [M+1]⁺. ¹H NMR (400 MHz, DMSO-d₆) δ=11.06 (s, 1H), 7.56 (d, J=8.4 Hz, 1H), 7.12 (t, J=5.2 Hz, 1H), 7.01 (d, J=1.2 Hz, 1H), 6.90 (dd, J=8.4, 1.6 Hz, 1H), 6.80 (s, 1H), 5.03 (dd, J=12.8, 5.2 Hz, 1H), 3.57 (t, J=5.6 Hz, 2H), 3.41 (t, J=6.0 Hz, 4H), 3.09 (q, J=5.6 Hz, 2H), 2.87 (m, 1H), 2.60-2.53 (m, 2H), 2.05-1.90 (m, 1H), 1.36 (s, 9H).

Procedure for preparation of compound 524-4. To a solution of compound 524-3 (90 mg, 195.45 μmol, 1 eq) in HBr (1 M, 586.35 uL, 40% purity, 3 eq). The mixture was stirred at 25° C. for 1 h. The reaction mixture was concentrated under the reduced pressure to give compound 524-4 (80 mg, crude) as a yellow solid. LCMS: t_{R}=0.222 min, MS (ESI+) m/z=361.2 [M+1]⁺

Procedure for preparation of compound 524-7. To a solution of compound 527-6 (181.82 mg, 222.00 μmol, 1 eq) and compound 524-4 (80 mg, 222.00 μmol, 1 eq) in DMAc (1 mL) was added DBU (101.39 mg, 665.99 μmol, 100.39 uL, 3 eq). The mixture was stirred at 80° C. for 1 h. The residue was purified by prep-HPLC (58%-88% ACN in water (10 mM NH₄HCO₃), 8 min) and lyophilized to give compound 524-7 (20 mg, 18.46 μmol, 8.32% yield) as a yellow solid. LCMS: t_{R}=0.884 min, MS (ESI+) m/z=1083.0 [M+1]⁺ Procedure for preparation of MS-5-024. To a solution of compound 527-7 (20 mg, 18.46 μmol, 1 eq) in DMSO (0.2 mL) was added CsF (14.02 mg, 92.31 μmol, 3.40 uL, 5 eq). The mixture was stirred at 50° C. for 1 h. The residue was purified by prep-HPLC (16%-46% ACN in water (0.225% FA), 10 min) and lyophilized to give MS-5-024 (2.95 mg, 3.17 μmol, 17.18% yield, 95.8% purity, FA) as a yellow solid. LCMS: t_{R}=0.348 min, MS (ESI+) m/z=845.5 [M+1]⁺. ¹H NMR (400 MHz, DMSO-d₆) δ=11.05 (s, 1H), 10.27 (s, 1H), 8.88 (d, J=7.6 Hz, 1H), 8.63 (s, 1H), 8.22 (s, 1H), 7.97 (s, 1H), 7.75-7.67 (m, 1H), 7.66-7.60 (m, 1H), 7.53 (d, J=8.4 Hz, 1H), 7.45-7.34 (m, 4H), 7.29-7.22 (m, 1H), 7.18-7.10 (m, 1H), 7.01 (d, J=2.0 Hz, 1H), 6.88 (dd, J=8.4, 1.2 Hz, 1H), 5.42 (dd, J=5.6, 3.6 Hz, 1H), 5.27-5.14 (m, 1H), 5.01 (dd, J=12.4, 4.8 Hz, 1H), 3.96-3.81 (m, 2H), 3.70-3.53 (m, 4H), 3.52-3.48 (m, 2H), 3.03 (dd, J=14.8, 5.2 Hz, 4H), 2.83 (dd, J=12.8, 5.6 Hz, 1H), 2.73 (t, J=4.8 Hz, 2H), 2.56 (d, J=2.0 Hz, 2H), 2.00-1.92 (m, 1H), 1.58 (s, 3H), 1.53 (s, 3H).

### Example 15

### Synthesis of MS-5-026

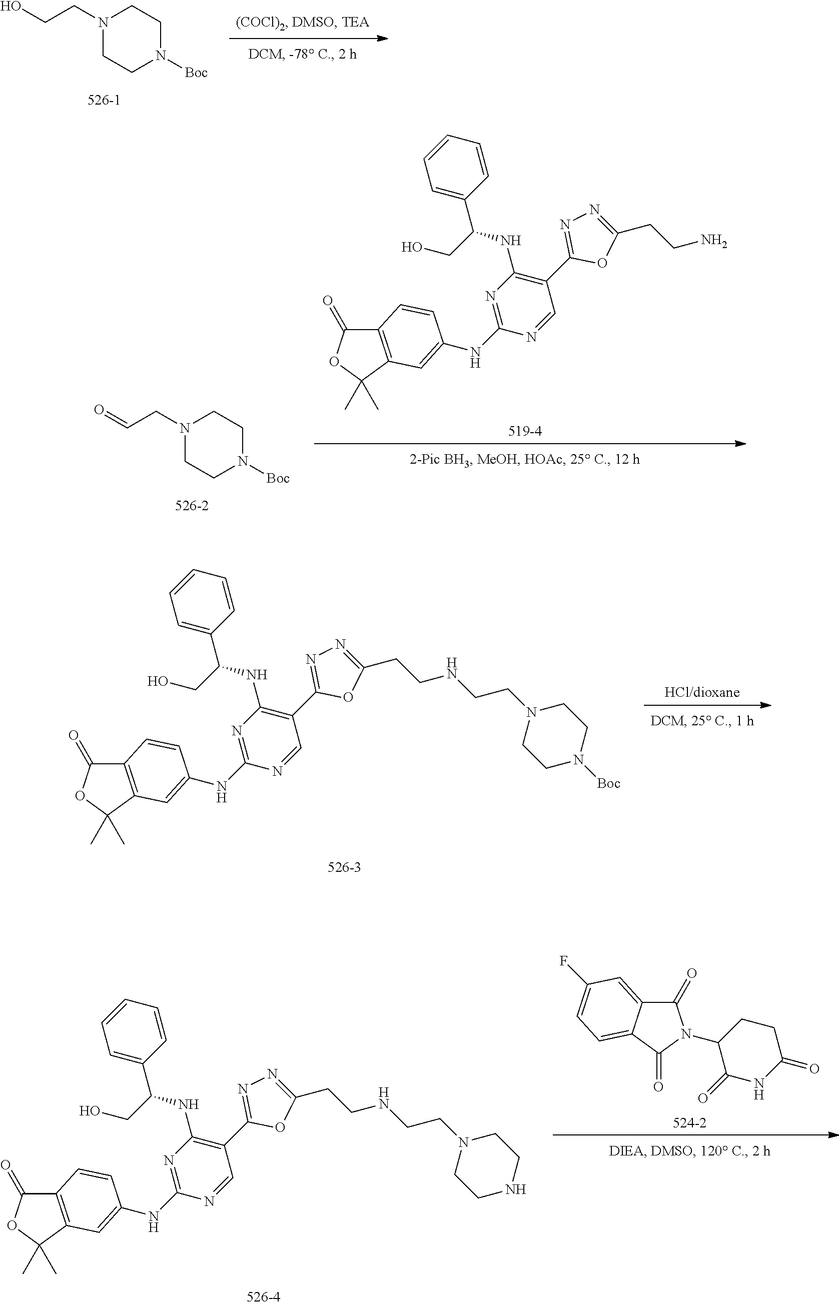

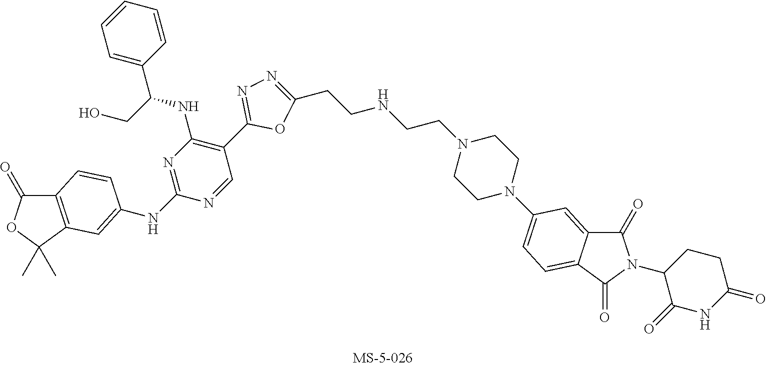 [SMILES: CC1(C)OC(=O)c2ccc(Nc3ncc(-c4nnc(CCNCCN5CCN(c6ccc7c(c6)C(=O)N(C6CCC(=O)NC6=O)C7=O)CC5)o4)c(N[C@H](CO)c4ccccc4)n3)cc21]

The procedure for preparing compound 519-4 is described in the synthesis of MS-5-019.

Procedure for preparation of compound 526-2. To a solution of DMSO (3.39 g, 43.42 mmol, 3.39 mL, 5 eq) in DCM (40 mL) was added a mixture of (COCl)₂ (2.20 g, 17.37 mmol, 1.52 mL, 2 eq) in DCM (40 mL) and stirred at −78° C. for 30 min and then compound 526-1 (commercially available, 2 g, 8.68 mmol, 1 eq) in DCM (40 mL) was added, then TEA (7.03 g, 69.47 mmol, 9.67 mL, 8 eq) was added and the resulting mixture was stirred at −78° C. for another 1.5 h. The reaction mixture was quenched by addition water (100 mL), and extracted with DCM (50 mL×3). The combined organic layers were dried over anhydrous Na₂SO₄, filtered and concentrated under reduced pressure to give compound 526-2 (1.9 g, crude) as a yellow oil. ¹H NMR (400 MHz, CDCl₃): δ=9.69 (t, J=1.2 Hz, 1H), 3.65-3.59 (m, 2H), 3.45-3.39 (m, 4H), 2.49-2.43 (m, 4H), 1.44 (s, 9H).

Procedure for preparation of compound 526-3. To a solution of compound 526-2 (195.15 mg, 854.84 μmol, 1.5 eq) and compound 519-4 (420 mg, 569.89 μmol, 73% purity, 1 eq, HCl) in MeOH (4 mL) and HOAc (0.4 mL) was added 2-Pic-BH₃ (60.96 mg, 569.89 μmol, 1 eq). The mixture was stirred at 25° C. for 12 h. The reaction mixture was concentrated under the reduced pressure to give a residue. The residue was purified by prep-HPLC (21%-41% ACN in water (0.05% HCl), 20 min) and lyophilized to give compound 526-3 (43 mg, 60.24 μmol, 10.57% yield) as a white solid. LCMS: t_{R}=0.395 min, MS (ESI+) m/z=714.2 [M+1]⁺. ¹H NMR (400 MHz, DMSO-d₆): δ=10.30 (s, 1H), 8.95-8.83 (m, 1H), 8.65 (s, 1H), 7.98 (s, 1H), 7.75-7.67 (m, 1H), 7.66-7.59 (m, 1H), 7.43-7.33 (m, 4H), 7.30-7.20 (m, 1H), 5.52-5.37 (m, 1H), 5.24 (s, 1H), 3.96-3.88 (m, 1H), 3.87-3.79 (m, 1H), 3.29 (s, 4H), 3.15-3.00 (m, 4H), 2.79-2.63 (m, 2H), 2.54 (s, 2H), 2.46-2.39 (m, 2H), 2.38-2.24 (m, 4H), 1.58 (s, 3H), 1.53 (s, 3H), 1.45-1.33 (m, 8H).

Procedure for preparation of compound 526-4. To a solution of compound 526-3 (43 mg, 60.24 μmol, 1 eq) in DCM (0.5 mL) was added HCl/dioxane (4 M, 45.18 uL, 3 eq). The mixture was stirred at 25° C. for 1 h. The reaction mixture was filtered and the filter residue was concentrated under the reduced pressure to give compound 526-4 (30 mg, crude) as a white solid. LCMS: t_{R}=0.326 min, MS (ESI+) m/z=614.3 [M+1]⁺.

Procedure for preparation of MS-5-026. To a solution of compound 526-4 (25 mg, 40.74 μmol, 1 eq) and compound 524-2 (commercially available, 11.25 mg, 40.74 μmol, 1 eq) in DMSO (0.5 mL) was added DIEA (7.90 mg, 61.10 μmol, 10.64 uL, 1.5 eq). The mixture was stirred at 120° C. for 2 h. The residue was purified by prep-HPLC (12%-42% ACN in water (0.1% TFA), 10 min) and lyophilized to give MS-5-026 (7.01 mg, 7.25 μmol, 17.80% yield, 90% purity) as a yellow solid. LCMS: t_{R}=0.373 min, MS (ESI+) m/z=870.5 [M+1]⁺. ¹H NMR (400 MHz, DMSO-d₆): δ=11.07 (s, 1H), 10.26 (s, 1H), 8.89 (d, J=7.8 Hz, 1H), 8.66 (s, 1H), 8.20 (s, 1H), 7.95 (s, 1H), 7.73-7.66 (m, 1H), 7.66-7.53 (m, 2H), 7.44-7.28 (m, 5H), 7.28-7.21 (m, 1H), 7.18-7.12 (m, 1H), 5.49-5.34 (m, 1H), 5.29-5.19 (m, 1H), 5.10-4.98 (m, 1H), 3.95-3.87 (m, 1H), 3.86-3.80 (m, 1H), 3.53-3.38 (m, 10H), 3.12-3.07 (m, 2H), 3.06-2.98 (m, 2H), 2.90-2.84 (m, 1H), 2.72 (t, J=5.6 Hz, 2H), 2.64-2.58 (m, 2H), 2.07-1.93 (m, 1H), 1.58 (s, 3H), 1.53 (s, 3H).

### Example 16

### Synthesis MS-5-027

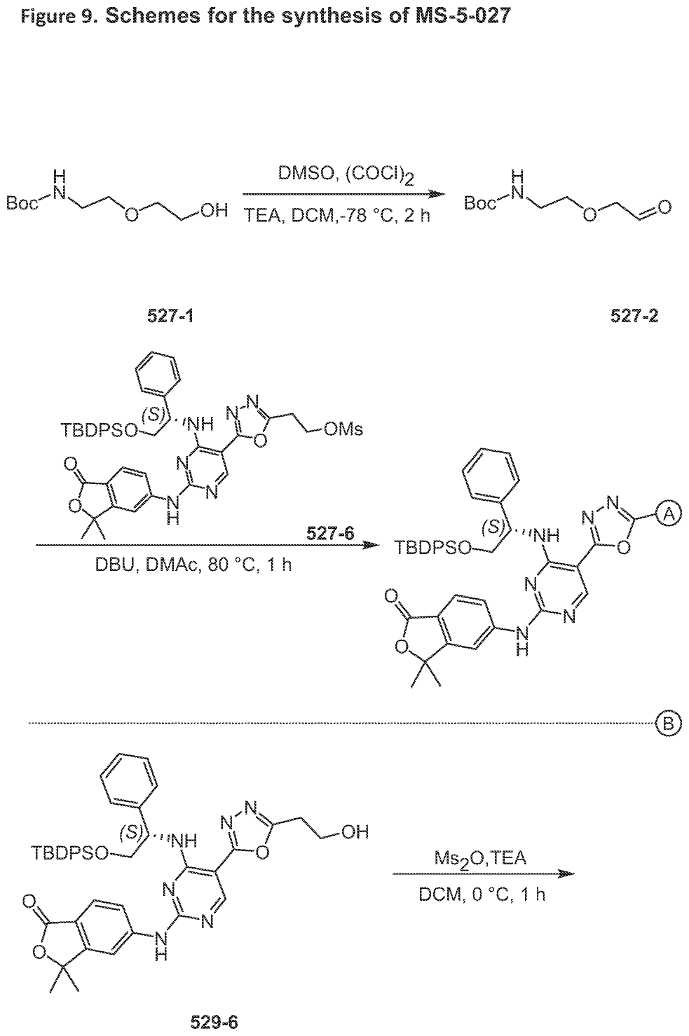

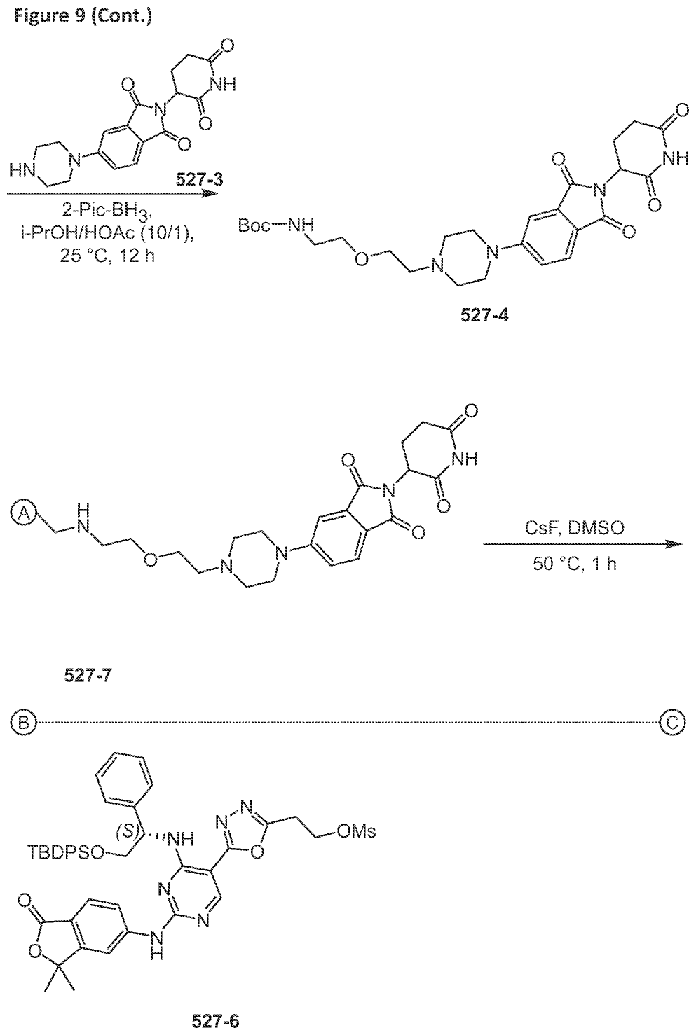

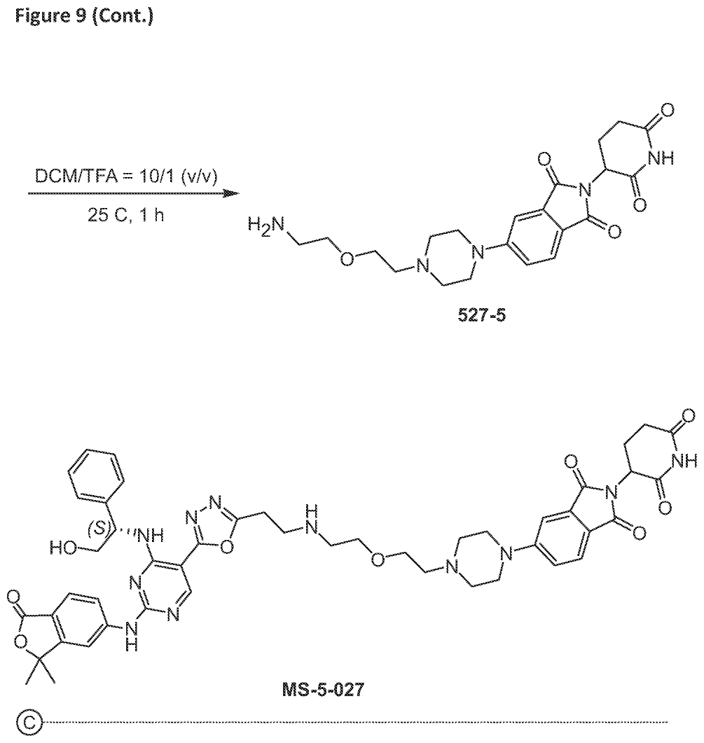

The reaction scheme for the synthesis of MS-5-027 is shown in FIG. 9.

The procedure for preparing compound 527-6 is described in the synthesis of MS-5-029.

The procedure for preparing compound 527-3 is described in the synthesis of MS-6-046.

Procedure for preparation of compound 527-2. To a solution of DMSO (1.90 g, 24.36 mmol, 1.90 mL, 5 eq) in DCM (20 mL) was added (COCl)₂ (1.24 g, 9.74 mmol, 852.96 uL, 2 eq) in DCM (20 mL) at −78° C. for 0.5 h. Then compound 527-1 (1 g, 4.87 mmol, 1 eq) in DCM (20 mL) was aeede, then TEA (3.94 g, 38.98 mmol, 5.43 mL, 8 eq) was added. The mixture was stirred at −78° C. for 1.5 h. The reaction mixture was partitioned between DCM (30 mL) and water (100 mL). After quenching the reaction, the reaction mixture was poured into separatory funnel and separated to give compound 527-2 (819 mg, crude) as a yellow oil. ¹H NMR (400 MHz, DMSO-d₆) δ=9.57 (s, 1H), 6.89-6.67 (m, 1H), 3.51-3.45 (m, 2H), 3.39 (d, J=7.6 Hz, 2H), 3.10-3.05 (m, 2H), 1.38 (s, 9H).

Procedure for preparation of compound 527-4. To a solution of compound 527-2 (445.24 mg, 2.19 mmol, 5 eq) and compound 527-3 (150 mg, 438.15 μmol, 1 eq) in HOAc (0.3 mL) and i-PrOH (3 mL) was added borane;2-methylpyridine (93.73 mg, 876.30 μmol, 2 eq). The mixture was stirred at 25° C. for 12 h. The residue was purified by prep-HPLC (2%-32% ACN in water (0.225% FA), 10 min) and lyophilized to give compound 527-4 (135 mg, 254.92 μmol, 58.18% yield) as a yellow solid. LCMS: t_{R}=0.317 min, MS (ESI+) m/z=530.2 [M+1]⁺. ¹H NMR (400 MHz, DMSO-d₆) δ=11.07 (s, 1H), 7.67 (d, J=8.4 Hz, 1H), 7.33 (s, 1H), 7.25 (d, J=8.4 Hz, 1H), 6.86-6.70 (m, 1H), 5.07 (dd, J=13.2, 5.2 Hz, 1H), 3.53 (t, J=5.2 Hz, 2H), 3.46-3.36 (m, 6H), 3.07 (q, J=5.2 Hz, 2H), 2.97-2.79 (m, 1H), 2.64-2.51 (m, 8H), 2.08-1.99 (m, 1H), 1.36 (s, 9H).

Procedure for preparation of compound 527-5. To a solution of compound 527-4 (130 mg, 245.48 μmol, 1 eq) in DCM (1 mL) was added TFA (0.1 mL). The mixture was stirred at 25° C. for 1 h. The reaction mixture was concentrated under the reduced pressure to give compound 527-(150 mg, crude) as a green oil. LCMS: t_{R}=0.171 min, MS (ESI+) m/z=430.1 [M+1]⁺

Procedure for preparation of compound 527-6. To a solution of compound 529-6 (1 g, 1.35 mmol, 1 eq) in DCM (10 mL) was added Ms₂O (705.32 mg, 4.05 mmol, 3 eq) and TEA (682.86 mg, 6.75 mmol, 939.29 uL, 5 eq) at 0° C. The mixture was stirred at 0° C. for 1 h. The reaction mixture was concentrated under the reduced pressure to give compound 527-6 (1.13 g, 1.10 mmol, 81.78% yield, 80% purity) as a yellow solid. LCMS: t_{R}=0.599 min, MS (ESI+) m/z=819.2 [M+1]⁺

Procedure for preparation of compound 527-7. To a solution of compound 527-5 (140 mg, 325.98 μmol, 1 eq) in DMAC (2 mL) was added DBU (148.88 mg, 977.95 μmol, 147.41 uL, 3 eq) and compound 527-6 (333.73 mg, 325.98 μmol, 80% purity, 1 eq) at 80° C. The mixture was stirred at 80° C. for 1 h. The residue was purified by prep-HPLC (34%-64% ACN in water (0.225% FA), 10 min) and lyophilized to give compound 527-7 (96 mg, 83.31 μmol, 25.56% yield) as a yellow solid. LCMS: t_{R}=0.461 min, MS (ESI+) m/z=1152.4 [M+1]⁺ Procedure for preparation of MS-5-027. To a solution of compound 527-7 (90 mg, 78.10 μmol, 1 eq) in DMSO (1 mL) was added CsF (59.32 mg, 390.50 μmol, 14.40 uL, 5 eq). The mixture was stirred at 50° C. for 1 h. The residue was purified by prep-HPLC (9%-39% ACN in water (0.225% FA), 10 min) and lyophilized to give MS-5-027 (38.29 mg, 39.49 μmol, 50.56% yield, 99% purity, FA) as a yellow solid. LCMS: t_{R}=0.331 min, MS (ESI+) m/z=914.4 [M+1]⁺. ¹H NMR (400 MHz, DMSO-d₆) δ=11.07 (s, 1H), 10.27 (s, 1H), 8.88 (d, J=7.6 Hz, 1H), 8.64 (s, 1H), 8.18 (s, 1H), 7.95 (d, J=1.2 Hz, 1H), 7.74-7.66 (m, 1H), 7.66-7.53 (m, 2H), 7.48-7.32 (m, 4H), 7.31-7.21 (m, 2H), 7.17 (dd, J=8.4, 2.0 Hz, 1H), 5.41-5.41 (m, 1H), 5.47-5.37 (m, 1H), 5.05 (dd, J=12.8, 5.6 Hz, 1H), 3.93-3.88 (m, 1H), 3.83 (dd, J=11.2, 4.8 Hz, 1H), 3.53 (s, 2H), 3.50 (s, 2H), 3.49 (s, 2H), 3.39-3.35 (m, 4H), 3.15-3.05 (m, 4H), 2.92-2.82 (m, 1H), 2.78 (t, J=5.6 Hz, 2H), 2.62-2.51 (m, 6H), 2.06-1.92 (m, 1H), 1.57 (s, 3H), 1.52 (s, 3H).

### Example 17

Synthetic Route for MS-5-028

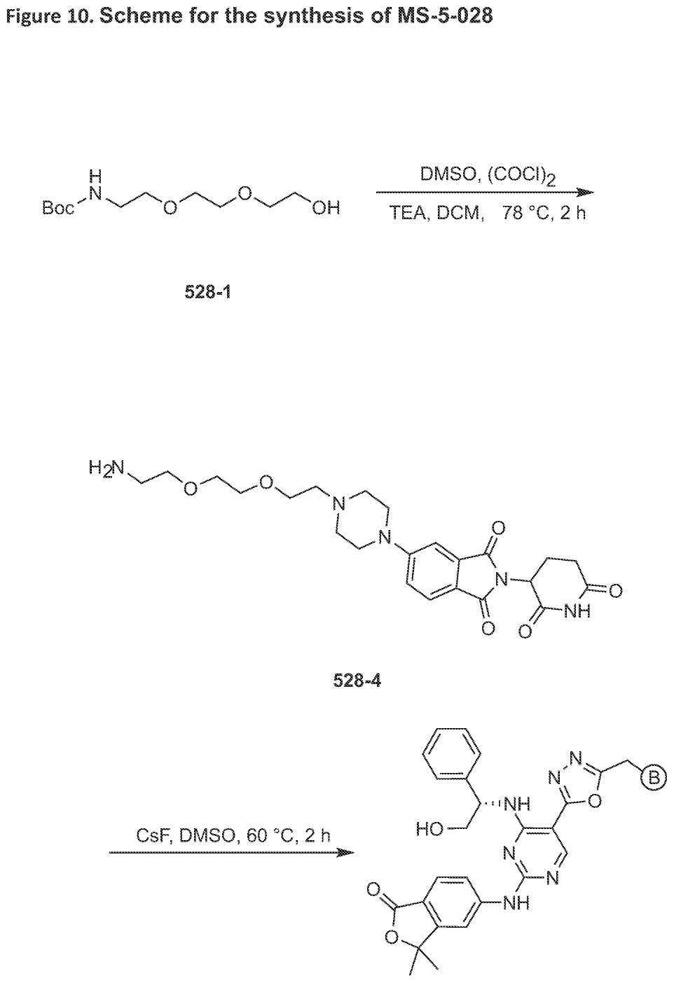

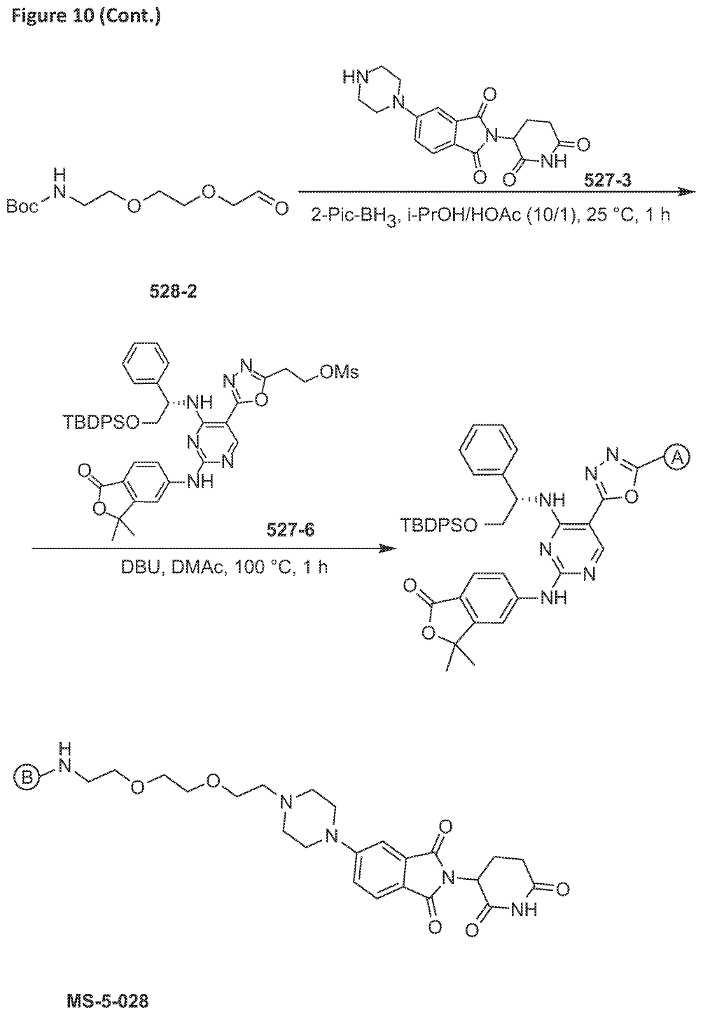

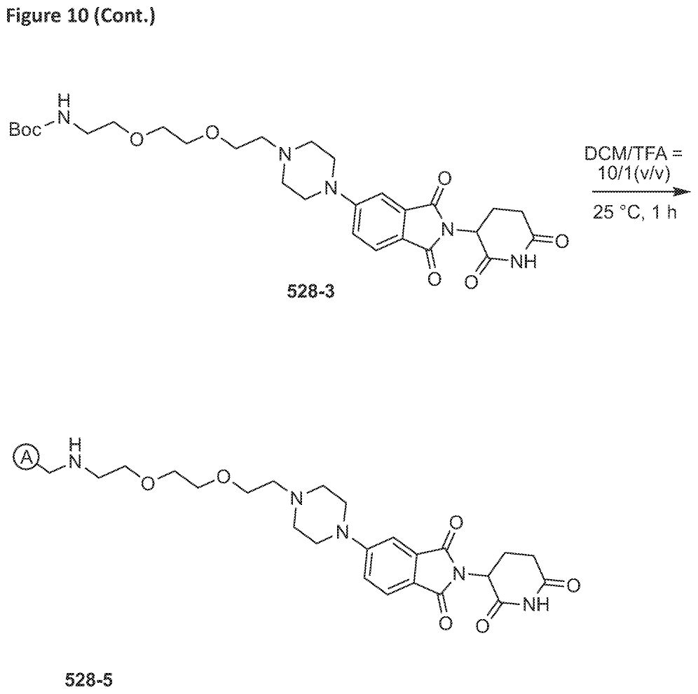

The reaction scheme for the synthesis of MS-5-028 is shown in FIG. 10.

The procedure for preparing compound 527-6 are described in the synthesis of MS-5-027.

The procedure for preparing compound 527-3 is described in the synthesis of MS-6-046.

Procedure for preparation of compound 528-2. To a solution of DMSO (1.57 g, 20.06 mmol, 1.57 mL, 5 eq) in DCM (20 mL) was added (COCl)₂ (1.02 g, 8.02 mmol, 702.24 μL, 2 eq) in DCM (20 mL) at −78° C. for 0.5 h. Then compound 528-1 (1 g, 4.01 mmol, 1 eq) in DCM (20 mL) was added, then TEA (3.25 g, 32.09 mmol, 4.47 mL, 8 eq) was added. The mixture was stirred at −78° C. for 1.5 h. The reaction mixture was partitioned between DCM (50 mL) and H₂O (50 mL). After quenching the reaction, the reaction mixture was poured into separatory funnel and separated to give compound 528-2 (1.45 g, crude) as a yellow oil. ¹H NMR (400 MHz, DMSO-d₆) δ=9.58 (s, 1H), 6.76 (d, J=5.6 Hz, 1H), 3.64-3.46 (m, 4H), 3.40-3.35 (m, 4H), 3.06 (d, J=7.6 Hz, 2H), 1.38 (s, 9H).

Procedure for preparation of compound 528-3. To a solution of compound 528-2 (1.44 g, 5.84 mmol, 5 eq) and compound 527-3 (400 mg, 1.17 mmol, 1 eq) in HOAc (0.5 mL) and i-PrOH (5 mL) was added 2-Pic-BH₃ (249.95 mg, 2.34 mmol, 2 eq). The mixture was stirred at 25° C. for 1 h. The residue was purified by prep-HPLC (4%-34% ACN in water (0.225% FA), 10 min) and lyophilized to give compound 528-3 (374 mg, 651.98 μmol, 55.80% yield) as a yellow solid. LCMS: t_{R}=0.315 min, MS (ESI+) m/z=574.3[M+1]⁺. ¹H NMR (400 MHz, DMSO-d₆) δ=11.07 (s, 1H), 7.67 (d, J=8.4 Hz, 1H), 7.33 (d, J=2.4 Hz, 1H), 7.25 (dd, J=8.8, 2.0 Hz, 1H), 6.81-6.67 (m, 1H), 5.14-4.97 (m, 1H), 3.55 (t, J=6.0 Hz, 2H), 3.50 (s, 4H), 3.44-3.38 (m, 6H), 3.15-2.99 (m, 2H), 2.98-2.79 (m, 1H), 2.65-2.51 (m, 8H), 2.06-1.99 (m, 1H), 1.36 (s, 9H).

Procedure for preparation of compound 528-4. To a solution of compound 528-3 (374 mg, 651.98 μmol, 1 eq) in DCM (3 mL) was added TFA (0.3 mL). The mixture was stirred at 25° C. for 1 h. The reaction mixture was concentrated under the reduced pressure to give compound 528-4 (701 mg, crude) as a yellow oil. LCMS: t_{R}=0.138 min, MS (ESI+) m/z=474.2[M+1]⁺.

Procedure for preparation of compound 528-5. To a solution of compound 528-4 (300 mg, 633.55 μmol, 1 eq) in DMAc (3 mL) was added DBU (289.35 mg, 1.90 mmol, 286.49 μL, 3 eq) and compound 527-6 (259.44 mg, 316.78 μmol, 0.5 eq) at 100° C. The mixture was stirred at 100° C. for 1 h. The residue was purified by prep-HPLC (23%-53% ACN in water (0.225% FA), 10 min) and lyophilized to give compound 528-5 (53 mg, 44.30 μmol, 6.99% yield) as a yellow solid. LCMS: t_{R}=0.470 min, MS (ESI+) m/z=1197.7[M+1]⁺.

Procedure for preparation of MS-5-028. To a solution of compound 528-5 (38 mg, 31.76 μmol, 1 eq) in DMSO (1 mL) was added CsF (4.82 mg, 31.76 μmol, 1.17 μL, 1 eq). The mixture was stirred at 60° C. for 2 h. The residue was purified by prep-HPLC (10%-40% ACN in water (0.225% FA), 8 min). The collected fractions were lyophilized to give MS-5-028 (9 mg, 8.81 μmol, 27.74% yield, 98.3% purity, FA) was obtained as a yellow solid. LCMS: t_{R}=0.327 min, MS (ESI+) m/z=958.8[M+1]⁺. ¹H NMR (400 MHz, DMSO-d₆) δ=11.07 (s, 1H), 10.28 (s, 1H), 8.89 (d, J=8.0 Hz, 1H), 8.64 (s, 1H), 8.18 (s, 1H), 7.96 (d, J=0.8 Hz, 1H), 7.76-7.67 (m, 1H), 7.63 (dd, J=2.4, 8.4 Hz, 2H), 7.43-7.34 (m, 4H), 7.31-7.23 (m, 2H), 7.19 (dd, J=2.1, 8.4 Hz, 1H), 5.48-5.37 (m, 1H), 5.34-5.13 (m, 1H), 5.05 (dd, J=5.6, 13.2 Hz, 1H), 3.90 (s, 1H), 3.86-3.82 (m, 1H), 3.51 (s, 14H), 3.19-2.97 (m, 8H), 2.88 (d, J=2.4 Hz, 1H), 2.76-2.71 (m, 2H), 2.61-2.54 (m, 2H), 2.06-1.94 (m, 1H), 1.57 (s, 3H), 1.52 (s, 3H).

### Example 18

Synthetic Route for MS-5-029

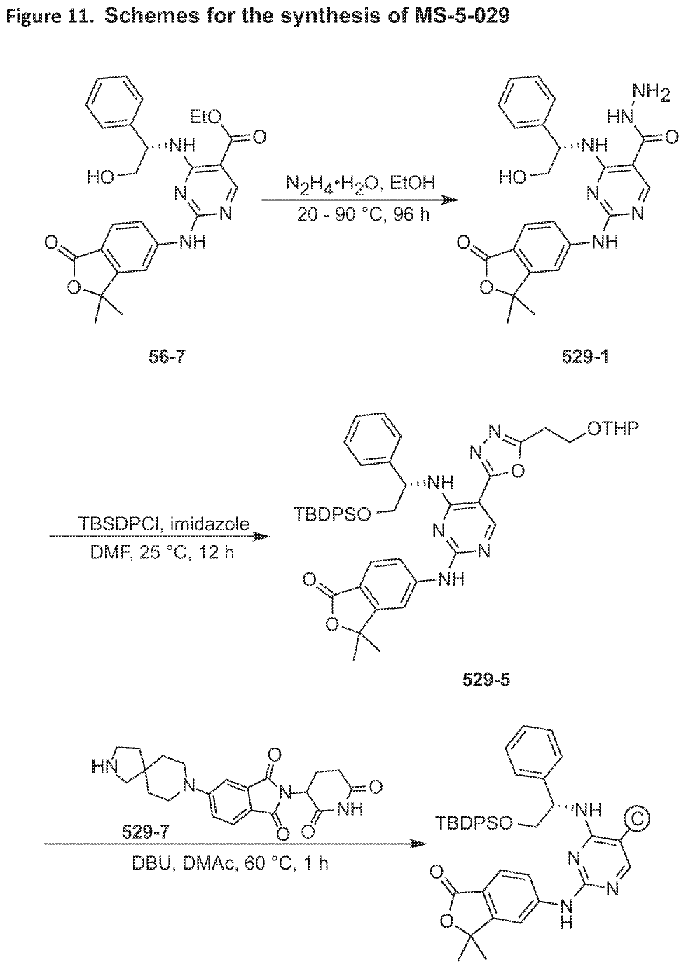

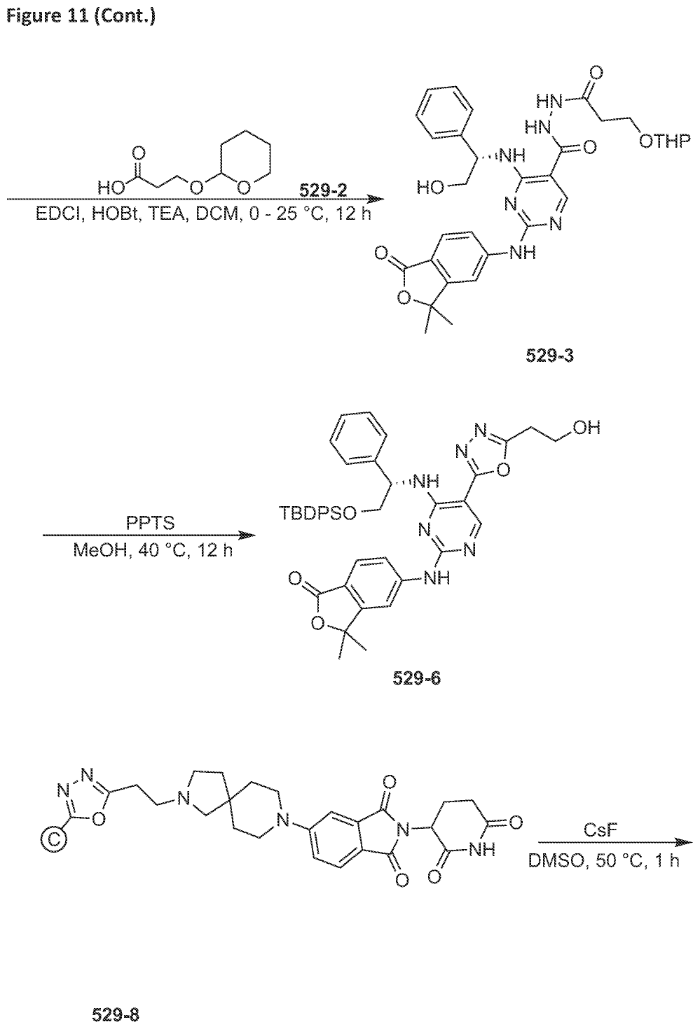

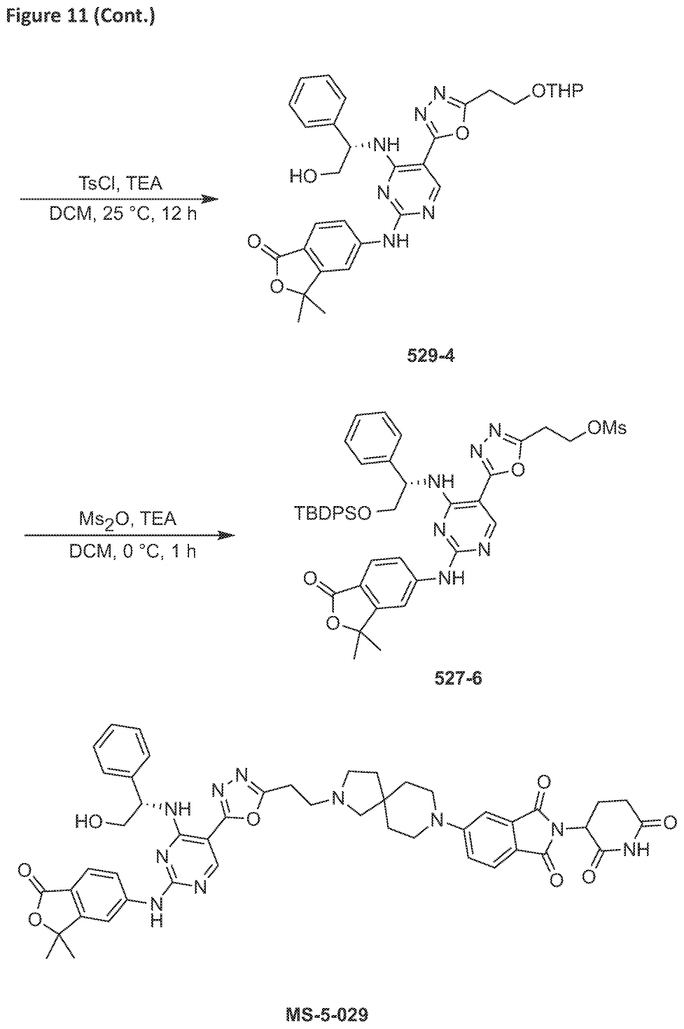

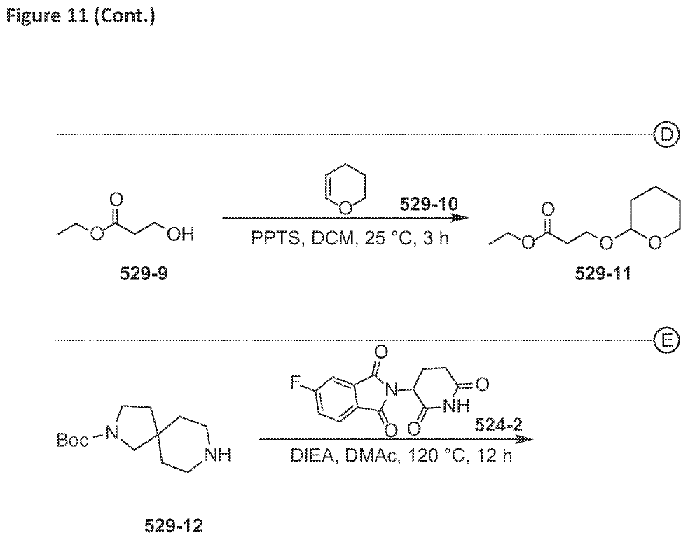

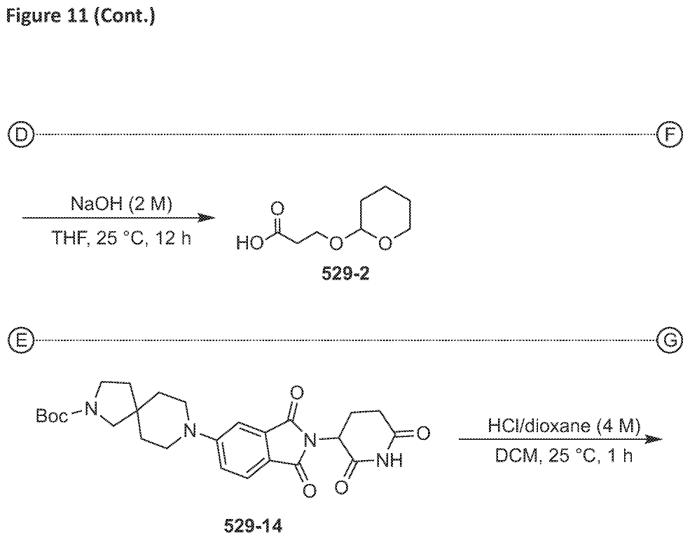

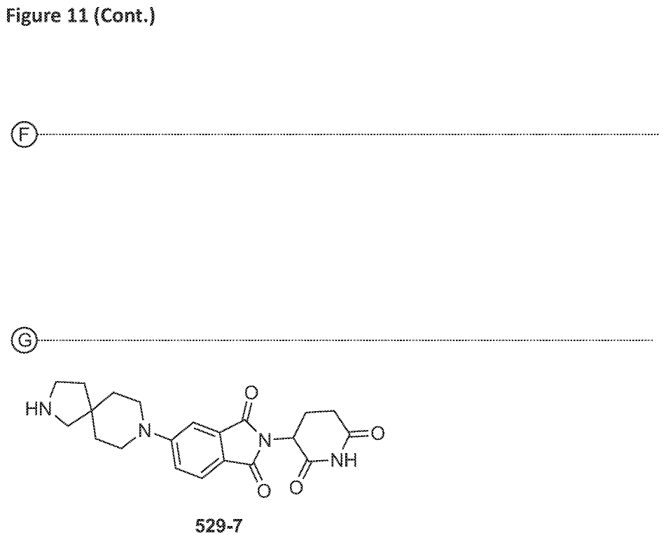

The reaction scheme for the synthesis of MS-5-029 is shown in FIG. 11.

The procedure for preparing compound 56-7 is described in the synthesis of MS-5-006.

Procedure for preparation of compound 529-1. To a solution of compound 56-7 (10 g, 21.62 mmol, 1 eq) in EtOH (500 mL) was added NH₂NH₂·H₂O (32.47 g, 648.65 mmol, 31.53 mL, 30 eq) slowly at 20° C. The mixture was stirred at 90° C. for 96 h. The reaction mixture was quenched by addition water (100 mL), and extracted with EtOAc (100 mL×3). The combined organic layers were dried over anhydrous Na₂SO₄, filtered and concentrated under reduced pressure to give a residue. The residue was purified by flash silica gel chromatography (SiO₂, DCM/MeOH=100/1 to 10/1) to give compound 529-1 (24.53 g, 54.71 mmol, 84.34% yield) as a white solid. LCMS: t_{R}=0.329 min, MS (ESI+) m/z=449.0 [M+1]⁺. ¹H NMR (400 MHz, DMSO-d₆) δ=10.04 (s, 1H), 9.76-9.60 (m, 2H), 8.53 (s, 1H), 7.96 (d, J=1.2 Hz, 1H), 7.72-7.64 (m, 1H), 7.64-7.58 (m, 1H), 7.42-7.31 (m, 4H), 7.30-7.21 (m, 1H), 5.28 (m, 1H), 5.13 (t, J=4.8 Hz, 1H), 4.44 (s, 2H), 3.93-3.80 (m, 1H), 3.79-3.69 (m, 1H), 1.57 (s, 3H), 1.53 (s, 3H).

Procedure for preparation of compound 529-12. To give compound 529-10 (25 g, 211.63 mmol, 1 eq) in DCM (250 mL) was added PPTS (2.66 g, 10.58 mmol, 0.05 eq) and compound 529-11 (24.92 g, 296.28 mmol, 27.09 mL, 1.4 eq). The mixture was stirred at 25° C. for 3 h. The reaction mixture was partitioned between DCM (150 mL×2) and water (150 mL). After quenching the reaction, the reaction mixture was poured into separatory funnel and separated. The residue was purified by flash silica gel chromatography (SiO₂, PE/EA=100/1 to 4/1) to give 529-12 (39.72 g, 196.39 mmol, 92.80% yield) as a colorless oil. ¹H NMR (400 MHz, CDCl₃) δ=4.63 (t, J=2.8 Hz, 1H), 4.23-4.07 (m, 2H), 4.00 (m, 1H), 3.86 (m, 1H), 3.69 (m, 1H), 3.56-3.45 (m, 1H), 2.60 (t, J=6.4 Hz, 2H), 1.89-1.76 (m, 1H), 1.73-1.64 (m, 1H), 1.62-1.43 (m, 4H), 1.30-1.22 (m, 3H).

Procedure for preparation of compound 529-2. To a solution of compound 529-12 (24.2 g, 119.66 mmol, 1 eq) in THE (250 mL) was added NaOH (2 M, 119.66 mL, 2 eq). The mixture was stirred at 25° C. for 12 h. The reaction mixture were washed with citric acid (5% in wt., 100 mL), and extracted with EtOAc (50 mL×3). The combined organic layers were dried over anhydrous Na₂SO₄, filtered and concentrated under reduced pressure to give compound 529-2 (14.54 g, 83.47 mmol, 69.76% yield) as a colourless oil. ¹H NMR (400 MHz, CDCl₃) δ=4.64 (t, J=3.2 Hz, 1H), 4.11 (q, J=7.2 Hz, 1H), 4.00 (m, 1H), 3.86 (m, 1H), 3.70 m, 1H), 3.57-3.47 (m, 1H), 2.65 (t, J=6.4 Hz, 2H), 1.90-1.75 (m, 1H), 1.74-1.65 (m, 1H), 1.64-1.43 (m, 4H).

Procedure for preparation of compound 529-3. To a solution of compound 529-2 (9.32 g, 53.51 mmol, 1 eq) in DCM (250 mL) was added EDCI (10.26 g, 53.51 mmol, 1 eq), TEA (5.96 g, 58.87 mmol, 8.19 mL, 1.1 eq) and HOBt (7.23 g, 53.51 mmol, 1 eq) at 0° C. for 30 min. Then compound 529-1 (24 g, 53.51 mmol, 1 eq) was added. The mixture was stirred at 25° C. for 11.5 h. The reaction mixture was partitioned between DCM (100 mL×2) and water (200 mL). After quenching the reaction, the reaction mixture was poured into separatory funnel and separated. The residue was purified by flash silica gel chromatography (SiO₂, DCM/MeOH=100/1 to 10/1) to give compound 529-3 (24.02 g, 39.73 mmol, 74.23% yield) as a white solid. LCMS: t_{R}=0.397 min, MS (ESI+) m/z=604.9 [M+1]⁺. ¹H NMR (400 MHz, DMSO-d₆): δ=10.25 (s, 1H), 10.14 (s, 1H), 9.86 (s, 1H), 9.55 (d, J=7.6 Hz, 1H), 8.68 (s, 1H), 7.96 (s, 1H), 7.72-7.66 (m, 1H), 7.65-7.60 (m, 1H), 7.36 (d, J=4.4 Hz, 4H), 7.29-7.20 (m, 1H), 5.30 (m, 1H), 5.14 (t, J=4.8 Hz, 1H), 4.66-4.51 (m, 1H), 3.95-3.69 (m, 4H), 3.68-3.57 (m, 1H), 3.49-3.39 (m, 1H), 2.49-2.45 (m, 1H), 1.79-1.60 (m, 2H), 1.58 (s, 3H), 1.53 (s, 3H), 1.51-1.33 (m, 4H).

Procedure for preparation of compound 529-4. To a solution of compound 529-3 (24 g, 39.69 mmol, 1 eq) in DCM (250 mL) was added TosCl (8.32 g, 43.66 mmol, 1.1 eq) and TEA (6.02 g, 59.54 mmol, 8.29 mL, 1.5 eq). The mixture was stirred at 25° C. for 12 h. The reaction mixture was partitioned between DCM (100 mL×2) and water (200 mL). Acidify the reaction mixture by adding, with shaking, 200 mL of NaHCO₃ until pH around 8, and then extracted with DCM (100 mL×2). The combined organic layers were dried over anhydrous Na₂SO₄, filtered and concentrated to give a residue. The residue was purified by flash silica gel chromatography (SiO₂, DCM/MeOH=100/1 to 10/1) to give compound 529-4 (19.57 g, 33.36 mmol, 84.05% yield) as a yellow solid. LCMS: t_{R}=0.464 min, MS (ESI+) m/z=587.1 [M+1]⁺. ¹H NMR (400 MHz, DMSO-d₆): δ=10.30 (s, 1H), 8.87 (d, J=7.6 Hz, 1H), 8.65 (s, 1H), 7.97 (s, 1H), 7.78-7.68 (m, 1H), 7.67-7.59 (m, 1H), 7.46-7.32 (m, 4H), 7.30-7.22 (m, 1H), 5.51-5.38 (m, 1H), 5.25 (t, J=4.4 Hz, 1H), 4.72-4.61 (m, 1H), 4.13-3.99 (m, 1H), 3.98-3.89 (m, 1H), 3.89-3.79 (m, 2H), 3.70 (m, 1H), 3.49-3.39 (m, 1H), 3.23 (t, J=6.4 Hz, 2H), 1.73-1.59 (m, 2H), 1.58 (s, 3H), 1.53 (s, 3H), 1.50-1.37 (m, 4H).

Procedure for preparation of compound 529-6. To a solution of compound 529-5 (19.5 g, 23.64 mmol, 1 eq) in MeOH (200 mL) was added PPTS (614.87 mg, 2.45 mmol, 1.04e-1 eq). The mixture was stirred at 40° C. for 12 h. The reaction mixture was concentrated under the reduced pressure to give a residue. The residue was purified by flash silica gel chromatography (SiO₂, DCM/MeOH=100/1 to 10/1) to give compound 529-6 (17.56 g, 22.75 mmol, 96.26% yield, 96% purity) as a white solid. LCMS: t_{R}=0.584 min, MS (ESI+) m/z=741.4 [M+1]⁺. ¹H NMR (400 MHz, DMSO-d₆): δ=9.21 (d, J=8.0 Hz, 1H), 8.53 (s, 1H), 7.62 (d, J=8.4 Hz, 1H), 7.56 (s, 1H), 7.54-7.48 (m, 3H), 7.45 (dd, J=8.4, 2.0 Hz, 1H), 7.33-7.17 (m, 13H), 5.51-5.30 (m, 1H), 4.09 (t, J=5.6 Hz, 2H), 4.00 (dd, J=4.2, 10.3 Hz, 1H), 3.89 (dd, J=10.4, 4.4 Hz, 1H), 3.13 (t, J=5.6 Hz, 2H), 2.70 (s, 1H), 1.53 (s, 6H), 0.93 (s, 9H).

Procedure for preparation of compound 527-6. To a solution of compound 529-6 (500 mg, 674.84 μmol, 1 eq) in DCM (5 mL) was added TEA (341.43 mg, 3.37 mmol, 469.64 uL, 5 eq) and MS₂O (352.66 mg, 2.02 mmol, 3 eq). The mixture was stirred at 0° C. for 1 h. The reaction mixture was partitioned between EtOAc (10 mL×3) and water (20 mL). After quenching the reaction, the reaction mixture was poured into separatory funnel and separated to give compound 527-6 (19.56 g, 23.71 mmol, 71.32% yield) as a colourless solid white solid. LCMS: t_{R}=0.604 min, MS (ESI+) m/z=819.0 [M+1]⁺

Procedure for preparation of compound 529-14. To a solution of compound 529-12 (500 mg, 2.08 mmol, 1 eq) and compound 524-2 (861.96 mg, 3.12 mmol, 1.5 eq) in DMAC (5 mL) was added DIEA (1.08 g, 8.32 mmol, 1.45 mL, 4 eq). The mixture was stirred at 120° C. for 12 h. The residue was purified by prep-HPLC (41%-71% ACN in water (0.225% FA), 10 min) and lyophilized to give compound 529-14 (885 mg, 1.78 mmol, 85.67% yield) as a yellow solid. LCMS: t_{R}=0.0.474 min, MS (ESI+) m/z=496.9 [M+1]⁺. ¹H NMR (400 MHz, CDCl₃) δ=8.06 (s, 1H), 7.69 (d, J=8.8 Hz, 1H), 7.29 (s, 1H), 7.07 (d, J=7.2 Hz, 1H), 4.95 (dd, J=12.4, 5.2 Hz, 1H), 3.64-3.15 (m, 8H), 2.97-2.65 (m, 3H), 2.21-2.09 (m, 1H), 1.79 (t, J=6.8 Hz, 2H), 1.75-1.66 (m, 4H), 1.48 (s, 9H).

Procedure for preparation of compound 529-7. To a solution of compound 529-14 (885 mg, 1.78 mmol, 1 eq) in DCM (10 mL) was added HCl/dioxane (4 M, 1.34 mL, 3 eq). The mixture was stirred at 25° C. for 1 h. The reaction mixture was filtered and the filter residue was concentrated under the reduced pressure to give compound 529-7 (634 mg, 1.46 mmol, 82.17% yield, HCl) as a yellow solid. LCMS: t_{R}=0.393 min, MS (ESI+) m/z=397.0 [M+1]⁺. ¹H NMR (400 MHz, DMSO-d₆): δ=11.07 (s, 1H), 9.36 (s, 2H), 7.66 (d, J=8.4 Hz, 1H), 7.36 (d, J=2.0 Hz, 1H), 7.27 (dd, J=8.8, 2.4 Hz, 1H), 5.06 (dd, J=12.8, 5.6 Hz, 1H), 3.55-3.42 (m, 4H), 3.30-3.19 (m, 2H), 3.03 (t, J=5.6 Hz, 2H), 2.96-2.79 (m, 1H), 2.65-2.52 (m, 2H), 2.08-1.94 (m, 1H), 1.85 (t, J=7.4 Hz, 2H), 1.72-1.50 (m, 4H).

Procedure for preparation of compound 529-9. To a solution of compound 527-6 (200 mg, 244.20 μmol, 1 eq) and compound 529-7 (158.57 mg, 366.30 μmol, 1.5 eq, HCl) in DMAC (6 mL) was added DBU (185.88 mg, 1.22 mmol, 184.04 uL, 5 eq). The mixture was stirred at 60° C. for 1 h. The residue was purified by prep-HPLC (42%-72% ACN in water (0.225% FA), 10 min) and lyophilized to give compound 529-8 (78 mg, 69.68 μmol, 28.54% yield) as a yellow solid. LCMS: t_{R}=0.527 min, MS (ESI+) m/z=1119.9 [M+1]⁺. ¹H NMR (400 MHz, DMSO-d₆): δ=11.07 (s, 1H), 10.30 (s, 1H), 9.24 (d, J=8.0 Hz, 1H), 8.69 (s, 1H), 7.89 (s, 1H), 7.73 (dd, J=8.4, 1.6 Hz, 1H), 7.66-7.59 (m, 2H), 7.54 (d, J=6.8 Hz, 2H), 7.45-7.36 (m, 6H), 7.36-7.23 (m, 8H), 7.22-7.15 (m, 1H), 5.65-5.52 (m, 1H), 5.06 (dd, J=12.8, 5.2 Hz, 1H), 4.15 (dd, J=10.8, 3.6 Hz, 1H), 3.98 (dd, J=10.0, 3.6 Hz, 1H), 3.49-3.37 (m, 4H), 3.15 (t, J=6.8 Hz, 2H), 2.95-2.81 (m, 3H), 2.70-2.52 (m, 6H), 2.08-1.96 (m, 1H), 1.63-1.51 (m, 11H), 0.92 (s, 9H).

Procedure for preparation of MS-5-029. To a solution of compound 529-8 (70 mg, 62.54 μmol, 1 eq) in DMSO (0.5 mL) was added CsF (95.00 mg, 625.37 μmol, 23.06 uL, 10 eq). The mixture was stirred at 50° C. for 1 h. The residue was purified by prep-HPLC (19%-49% ACN in water (0.225% FA), 9 min) and lyophilized to give MS-5-029 (20.5 mg, 22.12 μmol, 35.36% yield, 100% purity, FA) as a yellow solid. LCMS: t_{R}=0.397 min, MS (ESI+) m/z=881.4 [M+1]⁺. ¹H NMR (400 MHz, DMSO-d₆): δ=11.07 (s, 1H), 10.30 (s, 1H), 8.88 (d, J=7.6 Hz, 1H), 8.65 (s, 1H), 7.98 (s, 1H), 7.75-7.68 (m, 1H), 7.64 (d, J=8.8 Hz, 2H), 7.44-7.34 (m, 4H), 7.33-7.19 (m, 3H), 5.54-5.37 (m, 1H), 5.25 (s, 1H), 5.06 (dd, J=12.8, 5.6 Hz, 1H), 3.99-3.89 (m, 1H), 3.87-3.80 (m, 1H), 3.56-3.39 (m, 6H), 3.12 (t, J=6.8 Hz, 2H), 2.93-2.84 (m, 3H), 2.65-2.54 (m, 4H), 2.07-1.96 (m, 1H), 1.64-1.50 (m, 12H)

### Example 19

### Synthesis of MS-5-030

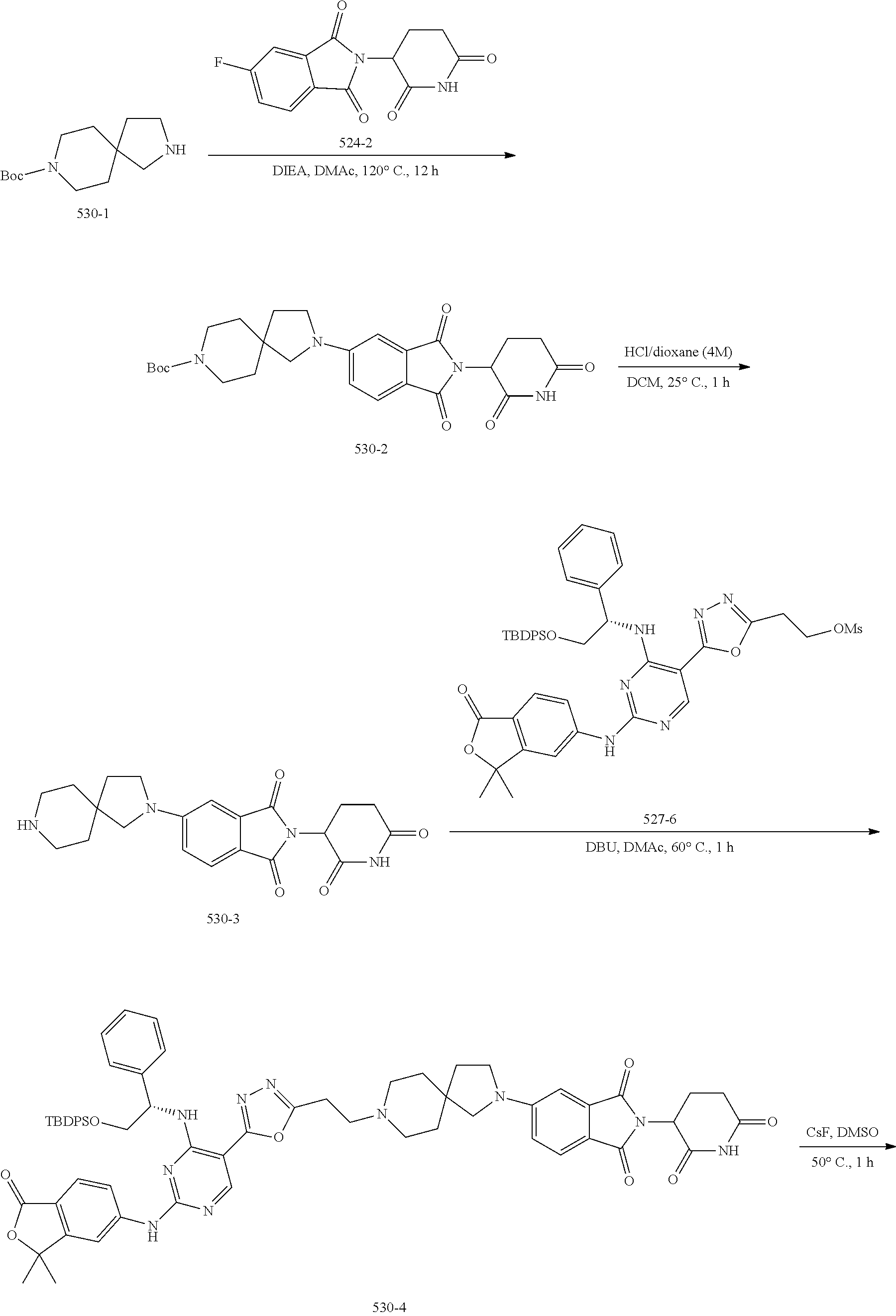

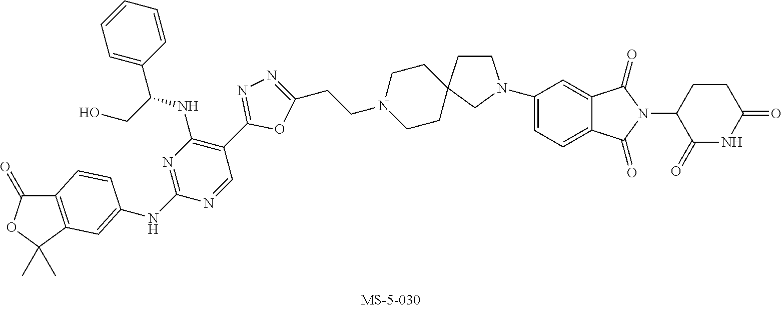 [SMILES: CC1(C)OC(=O)c2ccc(Nc3ncc(-c4nnc(CCN5CCC6(CC5)CCN(c5ccc7c(c5)C(=O)N(C5CCC(=O)NC5=O)C7=O)C6)o4)c(N[C@H](CO)c4ccccc4)n3)cc21]

The procedure for preparing compound 527-6 is described in the synthesis of MS-5-029.

Procedure for preparation of compound 530-2. To a solution of compound 530-1 (500 mg, 2.08 mmol, 1 eq) and compound 524-2 (commercially available, 861.96 mg, 3.12 mmol, 1.5 eq) in DMAc (5 mL) was added DIEA (1.08 g, 8.32 mmol, 1.45 mL, 4 eq). The mixture was stirred at 120° C. for 12 h. The residue was purified by prep-HPLC (3%-33% ACN in water (0.225% FA), 10 min) and lyophilized to give compound 529-2 (961 mg, 1.94 mmol, 93.03% yield) as a yellow solid. LCMS: t_{R}=0.479 min, MS (ESI+) m/z=440.9 [M+1]⁺. ¹H NMR (400 MHz, CDCl₃): δ=8.02 (s, 1H), 7.67 (d, J=8.8 Hz, 1H), 6.95 (d, J=2.0 Hz, 1H), 6.68 (dd, J=8.4, 2.0 Hz, 1H), 4.94 (dd, J=12.0, 5.2 Hz, 1H), 3.62-3.46 (m, 4H), 3.45-3.34 (m, 2H), 3.29 (s, 2H), 2.99-2.65 (m, 3H), 2.20-2.09 (m, 1H), 2.03-1.94 (m, 2H), 1.60 (s, 4H), 1.48 (s, 9H).

Procedure for preparation of compound 530-3. To give compound 530-2 (960 mg, 1.93 mmol, 1 eq) in DCM (10 mL) was added HCl/dioxane (4 M, 1.45 mL, 3 eq). The mixture was stirred at 25° C. for 1 h. The reaction mixture was filtered and the filter residue was concentrated under the reduced pressure to give compound 530-3 (854 mg, 1.62 mmol, 83.67% yield, 82% purity, HCl) as a yellow solid. LCMS: t_{R}=0.272 min, MS (ESI+) m/z=397.0 [M+1]⁺. ¹H NMR (400 MHz, DMSO-d₆) δ=11.06 (s, 1H), 9.13-8.67 (m, 2H), 7.65 (d, J=8.4 Hz, 1H), 6.96 (d, J=1.6 Hz, 1H), 6.84 (d, J=2.0 Hz, 1H), 5.05 (dd, J=12.8, 5.2 Hz, 1H), 3.54-3.44 (m, 4H), 3.40 (s, 2H), 3.11 (d, J=3.6 Hz, 3H), 2.96-2.81 (m, 1H), 2.63-2.51 (m, 2H), 2.08-1.86 (m, 3H), 1.75 (t, J=5.6 Hz, 3H).

Procedure for preparation of compound 530-4. To a solution of compound 530-3 (200 mg, 244.20 μmol, 1 eq) and compound 527-6 (158.57 mg, 366.30 μmol, 1.5 eq, HCl) in DMAc (4 mL) was added DBU (185.88 mg, 1.22 mmol, 184.04 uL, 5 eq). The mixture was stirred at 60° C. for 1 h. The residue was purified by prep-HPLC (41%-71% ACN in water (0.225% FA), 9 min) and lyophilized to give compound 530-4 (210 mg, 187.61 μmol, 76.83% yield) as a yellow solid. LCMS: t_{R}=0.524 min, MS (ESI+) m/z=1119.8 [M+1]⁺. ¹H NMR (400 MHz, DMSO-d₆): δ=11.06 (s, 1H), 10.30 (s, 1H), 9.23 (d, J=8.0 Hz, 1H), 8.69 (s, 1H), 7.90 (s, 1H), 7.73 (dd, J=8.4, 1.2 Hz, 1H), 7.62 (t, J=7.6 Hz, 2H), 7.56-7.50 (m, 2H), 7.45-7.37 (m, 6H), 7.35-7.26 (m, 7H), 6.92 (d, J=2.0 Hz, 1H), 6.80 (dd, J=8.4, 1.6 Hz, 1H), 5.65-5.53 (m, 1H), 5.05 (dd, J=12.4, 4.8 Hz, 1H), 4.22-4.08 (m, 1H), 3.98 (dd, J=10.0, 3.2 Hz, 1H), 3.45 (t, J=6.8 Hz, 4H), 3.17 (t, J=6.8 Hz, 2H), 2.91-2.80 (m, 3H), 2.63-2.54 (m, 4H), 2.06-1.96 (m, 1H), 1.86 (t, J=6.8 Hz, 2H), 1.59-1.46 (m, 11H), 0.92 (s, 9H).

Procedure for preparation of MS-5-030. To a solution of compound 530-4 (100 mg, 89.34 μmol, 1 eq) in DMSO (1 mL) was added CsF (135.71 mg, 893.38 μmol, 32.94 uL, 10 eq). The mixture was stirred at 50° C. for 1 h. The residue was purified by prep-HPLC (18%-48% ACN in water (0.225% FA), 9 min) and lyophilized to give MS-5-030 (24.83 mg, 27.75 μmol, 31.06% yield, 98.45% purity) as a yellow solid. LCMS: t_{R}=1.271 min, MS (ESI+) m/z=881.4 [M+1]⁺. ¹H NMR (400 MHz, DMSO-d₆): δ=11.06 (s, 1H), 10.29 (s, 1H), 8.88 (d, J=8.0 Hz, 1H), 8.65 (s, 1H), 8.23 (s, 1H), 7.98 (s, 1H), 7.80-7.67 (m, 1H), 7.63 (d, J=9.2 Hz, 2H), 7.49-7.32 (m, 4H), 7.31-7.19 (m, 1H), 6.92 (s, 1H), 6.81 (dd, J=8.8, 2.0 Hz, 1H), 5.53-5.37 (m, 1H), 5.34-5.13 (m, 1H), 5.05 (dd, J=12.8, 5.2 Hz, 1H), 3.98-3.88 (m, 1H), 3.88-3.79 (m, 1H), 3.48-3.44 (m, 4H), 3.14 (t, J=7.2 Hz, 2H), 2.91-2.79 (m, 3H), 2.62-2.53 (m, 6H), 2.04-1.95 (m, 1H), 1.88 (t, J=6.8 Hz, 2H), 1.56 (d, J=19.6 Hz, 10H).

### Example 20

### Synthesis of MS-5-033

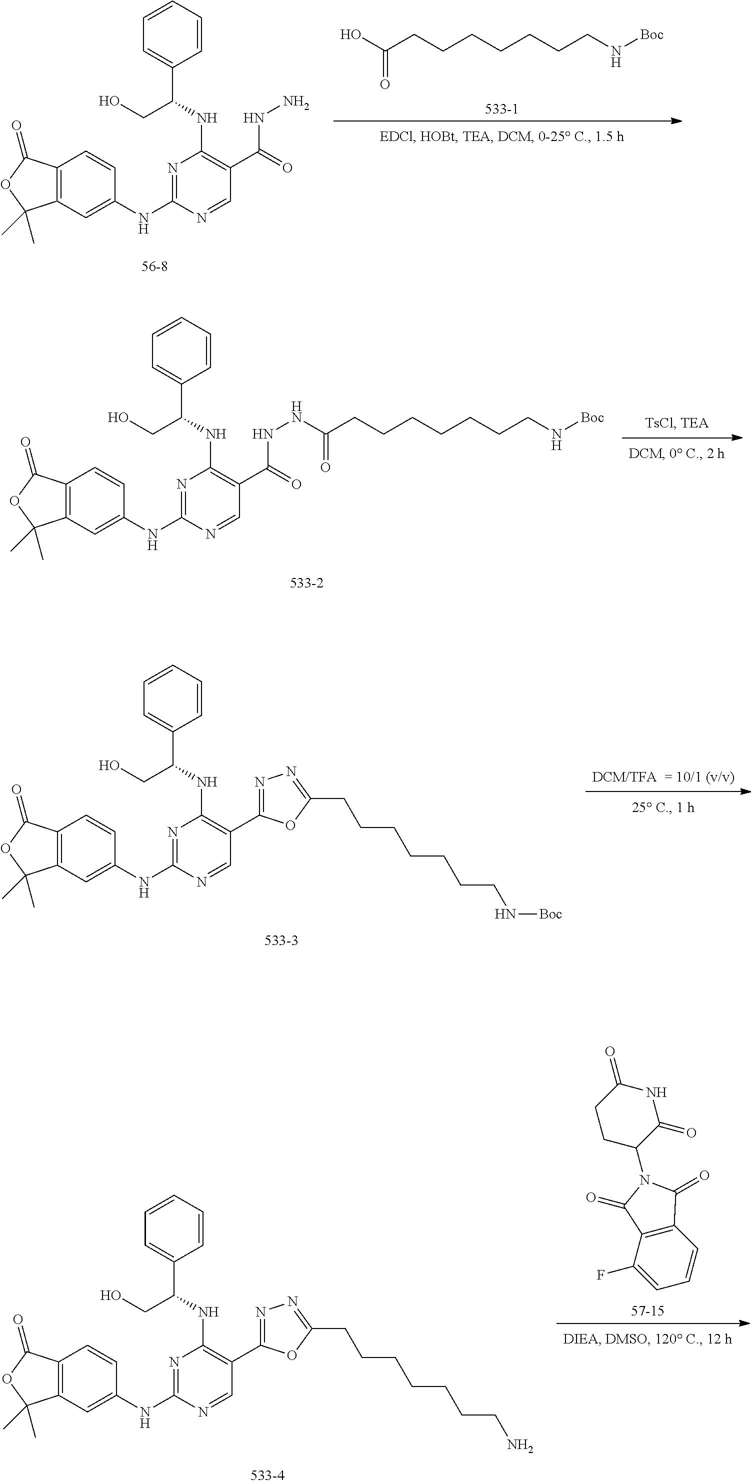

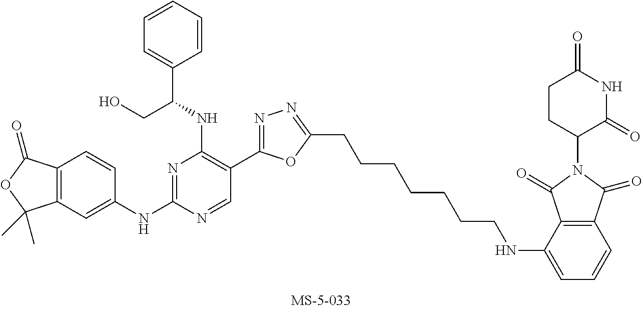 [SMILES: CC1(C)OC(=O)c2ccc(Nc3ncc(-c4nnc(CCCCCCCNc5cccc6c5C(=O)N(C5CCC(=O)NC5=O)C6=O)o4)c(N[C@H](CO)c4ccccc4)n3)cc21]

The procedure for preparing compound 56-8 is described in the synthesis of MS-5-006.

Procedure for preparation of compound 533-2. To a solution of compound 56-8 (900 mg, 3.47 mmol, 1.2 eq) in DCM (10 mL) was added EDCI (831.59 mg, 4.34 mmol, 1.5 eq), HOBt (390.77 mg, 2.89 mmol, 1 eq) and TEA (877.90 mg, 8.68 mmol, 1.21 mL, 3 eq). The mixture was stirred at 0° C. for 0.5 h. Then compound 533-1 (1.30 g, 2.89 mmol, 1 eq) was added, the mixture was stirred at 25° C. for 1 h. The reaction mixture was partitioned between DCM (10 mL×3) and water (10 mL). After quenching the reaction, the reaction mixture was poured into separatory funnel and separated. The residue was purified by prep-HPLC (20%-50% ACN in water (0.225% FA), 30 min) and lyophilized to give compound 533-2 (903 mg, 1.31 mmol, 45.27% yield) as a white solid. LCMS: t_{R}=0.449 min, MS (ESI+) m/z=690.4 [M+1]⁺. ¹H NMR (400 MHz, DMSO-d₆) δ=10.33-10.03 (m, 2H), 9.75 (s, 1H), 9.54 (d, J=6.0 Hz, 1H), 8.66 (s, 1H), 7.96 (d, J=1.2 Hz, 1H), 7.75-7.65 (m, 1H), 7.65-7.58 (m, 1H), 7.35 (d, J=4.4 Hz, 3H), 7.25 (m, 1H), 6.83-6.68 (m, 1H), 5.36-5.23 (m, 1H), 5.14 (t, J=5.2 Hz, 1H), 3.89-3.80 (m, 1H), 3.79-3.70 (m, 1H), 2.90 (q, J=6.8 Hz, 2H), 2.18 (t, J=7.6 Hz, 2H), 1.55 (d, J=18.8 Hz, 7H), 1.41-1.21 (m, 17H).

Procedure for preparation of compound 533-3. To a solution of compound 533-2 (400 mg, 579.88 μmol, 1 eq) in DCM (5 mL) was added TosCl (121.61 mg, 637.87 μmol, 1.1 eq) and TEA (88.02 mg, 869.82 μmol, 121.07 uL, 1.5 eq). The mixture was stirred at 0° C. for 2 h. The reaction mixture was concentrated under the reduced pressure and purified by prep-HPLC (52%-82% ACN in water (0.225% FA), 10 min) and lyophilized to give compound 533-3 (251 mg, 373.63 μmol, 64.43% yield) as a white solid. LCMS: t_{R}=0.521 min, MS (ESI+) m/z=672.4 [M+1]⁺. ¹H NMR (400 MHz, DMSO-d₆) δ=10.29 (s, 1H), 8.88 (d, J=7.6 Hz, 1H), 8.64 (s, 1H), 7.98 (s, 1H), 7.76-7.67 (m, 1H), 7.66-7.59 (m, 1H), 7.47-7.32 (m, 4H), 7.31-7.21 (m, 1H), 6.76 (t, J=5.2 Hz, 1H), 5.50-5.38 (m, 1H), 5.24 (s, 1H), 3.98-3.88 (m, 1H), 3.88-3.78 (m, 1H), 2.99-2.83 (m, 4H), 1.77 (m, 2H), 1.58 (s, 3H), 1.53 (s, 3H), 1.42-1.24 (m, 17H).

Procedure for preparation of compound 533-4. To a solution of compound 533-3 (240 mg, 357.26 μmol, 1 eq) in DCM (2 mL) and TFA (0.2 mL). The mixture was stirred at 25° C. for 1 h. The reaction mixture was quenched by addition ammonium hydroxide (1 mL) and was concentrated under the reduced pressure to give compound 533-4 (318 mg, crude) as a white solid. LCMS: t_{R}=0.391 min, MS (ESI+) m/z=572.3 [M+1]⁺ Procedure for preparation of MS-5-033. To a solution of compound 533-4 (200 mg, 349.85 μmol, 1 eq) and compound 57-15 (115.96 mg, 419.82 μmol, 1.2 eq) in DMSO (1 mL) was added DIEA (90.43 mg, 699.71 μmol, 121.88 uL, 2 eq). The mixture was stirred at 120° C. for 12 h. The residue was purified by prep-HPLC (52%-82% ACN in water (0.225% FA), 10 min) and lyophilized to give MS-5-033 (47.89 mg, 52.61 μmol, 15.04% yield, 96% purity, FA) as a yellow solid. LCMS: t_{R}=0.517 min, MS (ESI+) m/z=828.3 [M+1]⁺. ¹H NMR (400 MHz, DMSO-d₆) δ=11.09 (s, 1H), 10.29 (s, 1H), 8.88 (d, J=8.0 Hz, 1H), 8.64 (s, 1H), 8.15 (s, 1H), 7.97 (s, 1H), 7.74-7.67 (m, 1H), 7.66-7.61 (m, 1H), 7.60-7.53 (m, 1H), 7.44-7.34 (m, 4H), 7.30-7.22 (m, 1H), 7.09 (d, J=8.4 Hz, 1H), 7.01 (d, J=6.8 Hz, 1H), 6.54 (t, J=5.6 Hz, 1H), 5.48-5.36 (m, 1H), 5.23 (t, J=4.8 Hz, 1H), 5.04 (dd, J=13.2, 5.2 Hz, 1H), 3.95-3.88 (m, 1H), 3.87-3.79 (m, 1H), 2.94 (t, J=7.6 Hz, 2H), 2.88-2.82 (m, 1H), 2.59 (d, J=2.4 Hz, 2H), 2.06-1.97 (m, 1H), 1.83-1.75 (m, 2H), 1.64-1.33 (m, 16H).

### Example 21

### Synthetic Route for MS-5-034

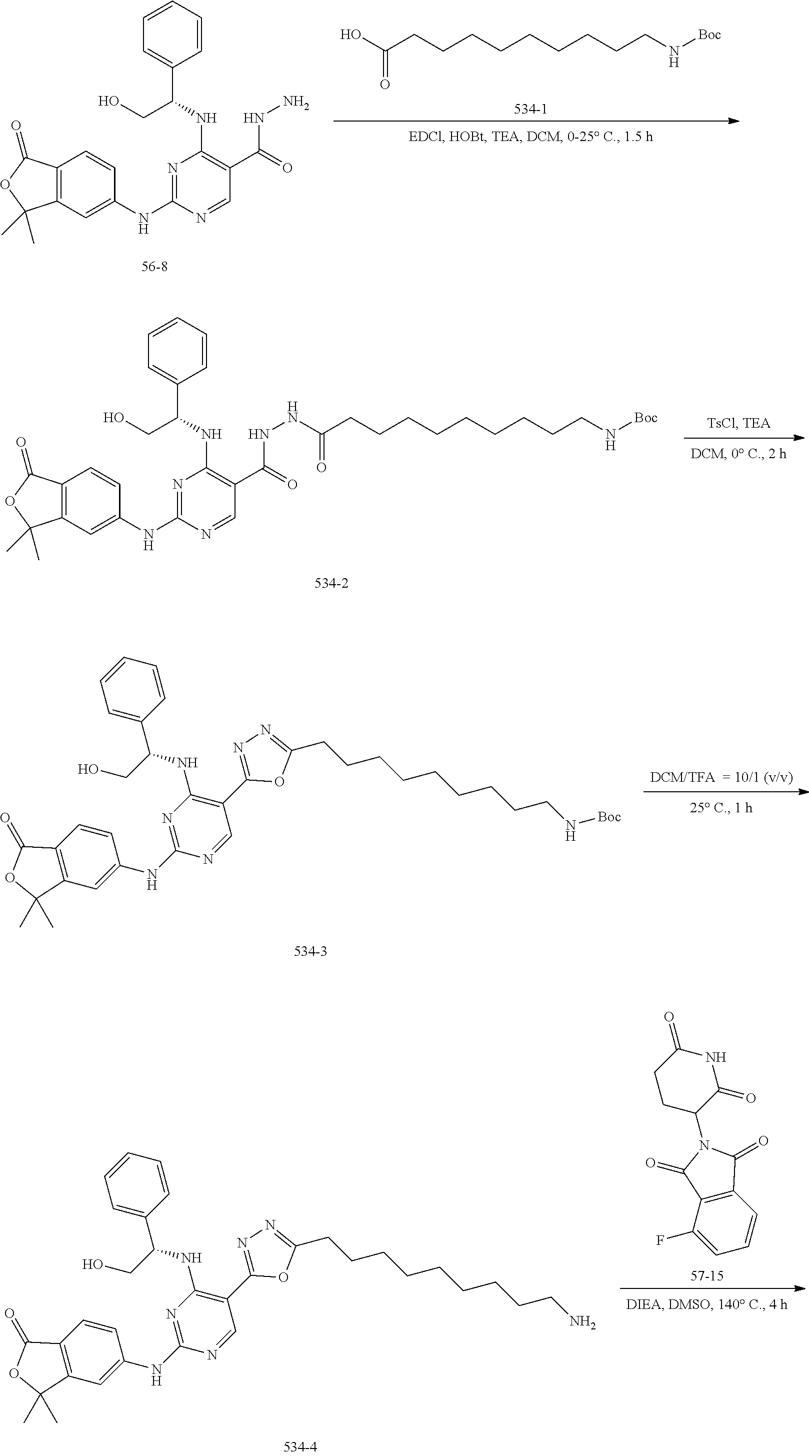

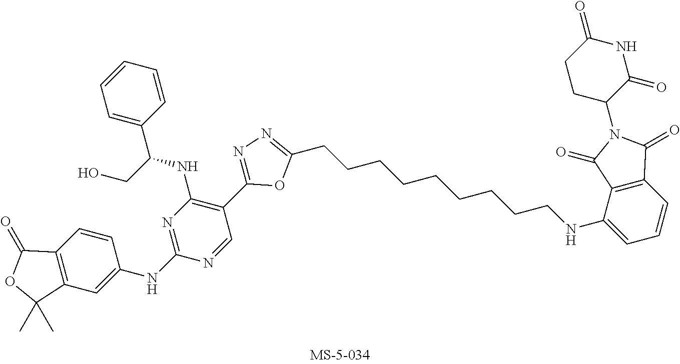 [SMILES: CC1(C)OC(=O)c2ccc(Nc3ncc(-c4nnc(CCCCCCCCCNc5cccc6c5C(=O)N(C5CCC(=O)NC5=O)C6=O)o4)c(N[C@H](CO)c4ccccc4)n3)cc21]

The procedure for preparing compound 56-8 is described in the synthesis of MS-5-006.

Procedure for preparation of compound 534-2. To a solution of compound 56-8 (900 mg, 3.13 mmol, 1.2 eq) in DCM (10 mL) was added EDCI (750.42 mg, 3.91 mmol, 1.5 eq), HOBt (352.62 mg, 2.61 mmol, 1 eq) and TEA (792.21 mg, 7.83 mmol, 1.09 mL, 3 eq). The mixture was stirred at 0° C. for 0.5 h. Then compound 534-1 (1.17 g, 2.61 mmol, 1 eq) was added, the mixture was stirred at 25° C. for 1 h. The reaction mixture was partitioned between DCM (10 mL×3) and water (10 mL). After quenching the reaction, the reaction mixture was poured into separatory funnel and separated. The residue was purified by prep-HPLC (30%-60% ACN in water (0.225% FA), 30 min) and lyophilized to give compound 534-2 (907 mg, 1.26 mmol, 48.42% yield) as a white solid. LCMS: t_{R}=0.473 min, MS (ESI+) m/z=718.7 [M+1]⁺. ¹H NMR (400 MHz, DMSO-d₆) δ=10.25-10.05 (m, 2H), 9.75 (s, 1H), 9.54 (d, J=7.6 Hz, 1H), 8.67 (s, 1H), 7.96 (s, 1H), 7.73-7.65 (m, 1H), 7.65-7.58 (m, 1H), 7.35 (d, J=4.4 Hz, 3H), 7.25 (m, 1H), 6.75 (t, J=4.8 Hz, 1H), 5.29 (m, 1H), 5.14 (t, J=4.4 Hz, 1H), 3.89-3.80 (m, 1H), 3.75 (m, 1H), 2.88 (q, J=6.8 Hz, 2H), 2.18 (t, J=7.2 Hz, 2H), 1.55 (d, J=18.4 Hz, 8H), 1.38-1.20 (m, 21H).

Procedure for preparation of compound 534-3. To a solution of compound 534-2 (400 mg, 557.22 μmol, 1 eq) in DCM (5 mL) was added TosCl (116.86 mg, 612.94 μmol, 1.1 eq) and TEA (84.58 mg, 835.83 μmol, 116.34 uL, 1.5 eq) at 0° C. and the mixture was stirred at this temperature for 2 h. The reaction mixture was concentrated under the reduced pressure and purified by prep-HPLC (62%-92% ACN in water (0.225% FA), 10 min) and lyophilized to give compound 534-3 (261 mg, 372.94 μmol, 66.93% yield) as a white solid. LCMS: t_{R}=0.549 min, MS (ESI+) m/z=700.8 [M+1]⁺. ¹H NMR (400 MHz, DMSO-d₆) δ=10.29 (s, 1H), 8.89 (d, J=7.6 Hz, 1H), 8.64 (s, 1H), 7.98 (s, 1H), 7.77-7.67 (m, 1H), 7.66-7.61 (m, 1H), 7.44-7.32 (m, 4H), 7.31-7.22 (m, 1H), 6.75 (t, J=4.8 Hz, 1H), 5.55-5.37 (m, 1H), 5.24 (s, 1H), 3.96-3.88 (m, 1H), 3.87-3.80 (m, 1H), 2.97-2.83 (m, 4H), 1.77 (m, 2H), 1.58 (s, 3H), 1.53 (s, 3H), 1.40-1.21 (m, 21H).

Procedure for preparation of compound 534-4. To a solution of compound 534-3 (200 mg, 285.78 μmol, 1 eq) in DCM (5 mL) and TFA (0.5 mL). The mixture was stirred at 25° C. for 1 h. The reaction mixture was quenched by addition ammonium hydroxide (2 mL) and was concentrated under the reduced pressure to give compound 534-4 (210 mg, crude) as a yellow solid. LCMS: t_{R}=0.417 min, MS (ESI+) m/z=600. [M+1]⁺ Procedure for preparation of MS-5-034. To a solution of compound 534-4 (100 mg, 166.74 μmol, 1 eq) and compound 57-15 (55.27 mg, 200.09 μmol, 1.2 eq) in DMSO (2 mL) was added DIEA (43.10 mg, 333.49 μmol, 58.09 uL, 2 eq). The mixture was stirred at 140° C. for 4 h. The residue was purified by prep-HPLC (41%-71% ACN in water (0.225% FA), 10 min) and prep-HPLC (52%-82% ACN in water (0.225% FA), 10 min) and lyophilized to give MS-5-034 (12.96 mg, 14.38 μmol, 8.63% yield, 95% purity) as a yellow solid. LCMS: t_{R}=0.547 min, MS (ESI+) m/z=856.3 [M+1]⁺. ¹H NMR (400 MHz, DMSO-d₆) δ=11.09 (s, 1H), 10.28 (s, 1H), 8.88 (d, J=8.0 Hz, 1H), 8.64 (s, 1H), 7.97 (s, 1H), 7.75-7.68 (m, 1H), 7.66-7.61 (m, 1H), 7.56 (dd, J=8.4, 7.2 Hz, 1H), 7.42-7.34 (m, 4H), 7.30-7.23 (m, 1H), 7.08 (d, J=8.8 Hz, 1H), 7.00 (d, J=7.2 Hz, 1H), 6.52 (t, J=5.6 Hz, 1H), 5.49-5.37 (m, 1H), 5.23 (t, J=4.8 Hz, 1H), 5.04 (dd, J=12.8, 5.2 Hz, 1H), 3.97-3.88 (m, 1H), 3.87-3.78 (m, 1H), 3.02-2.78 (m, 3H), 2.62-2.53 (m, 2H), 2.06-1.99 (m, 1H), 1.77 (m, 2H), 1.55 (d, J=20.0 Hz, 8H), 1.41-1.23 (m, 12H).

### Example 22

### Synthesis of MS-5-035

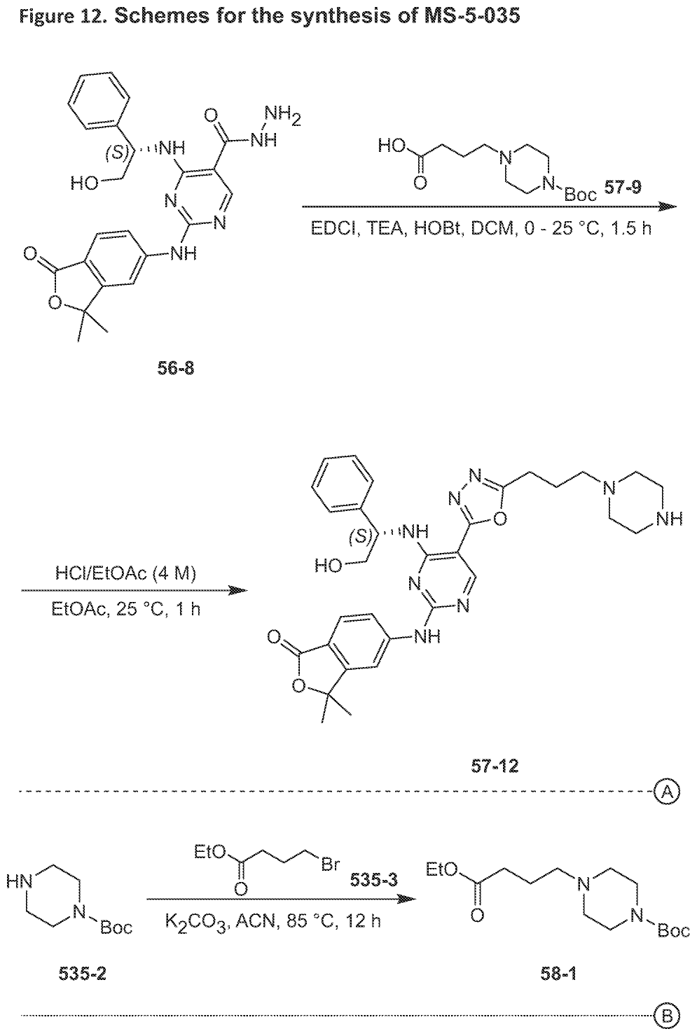

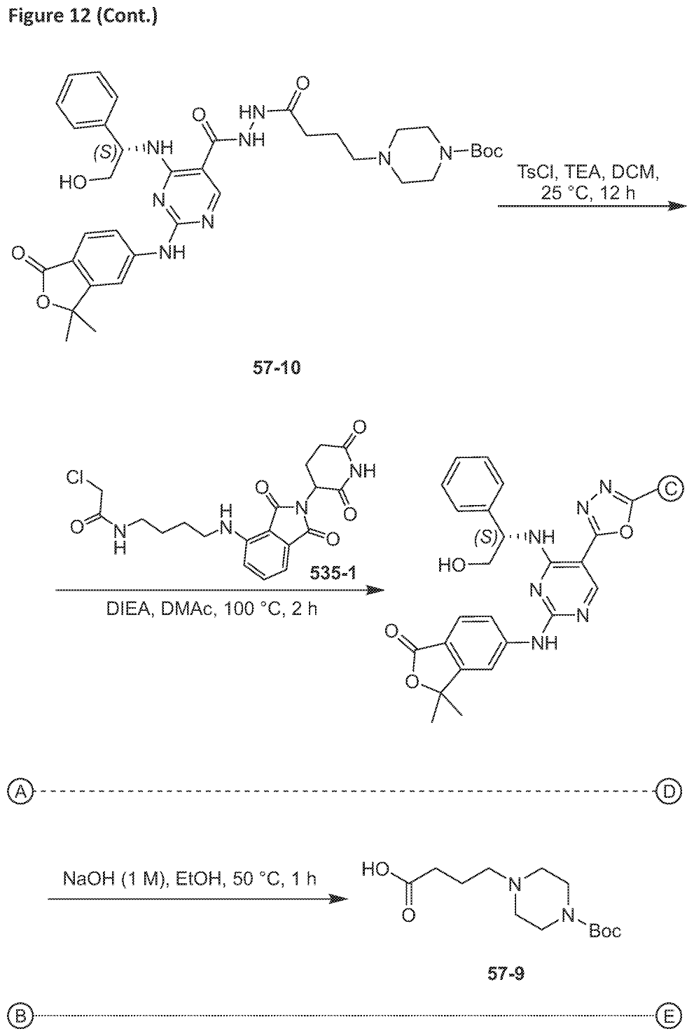

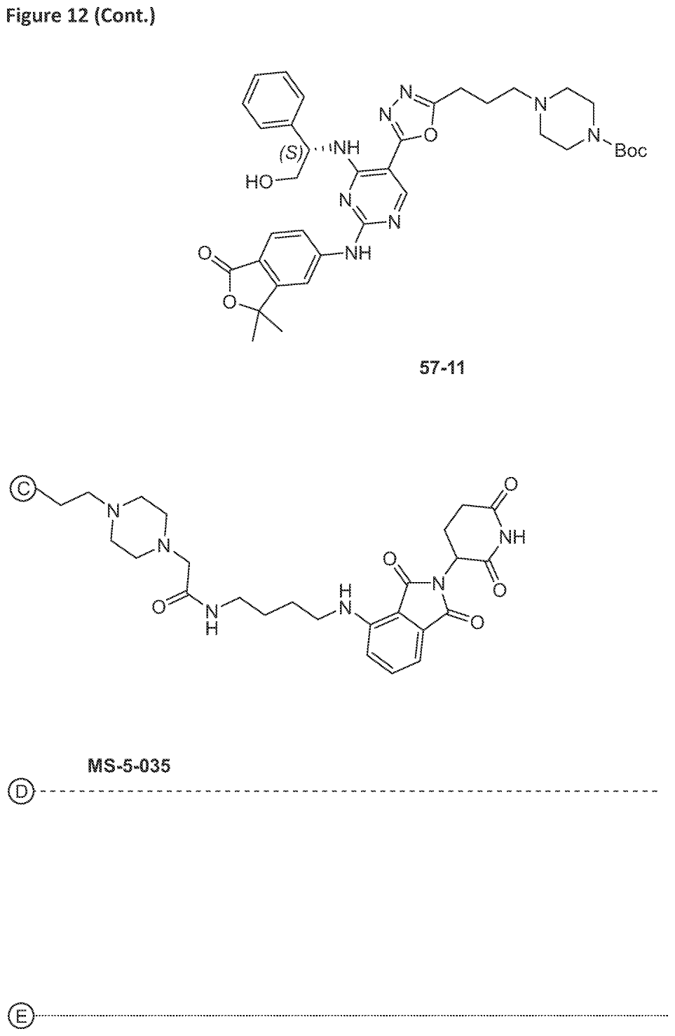

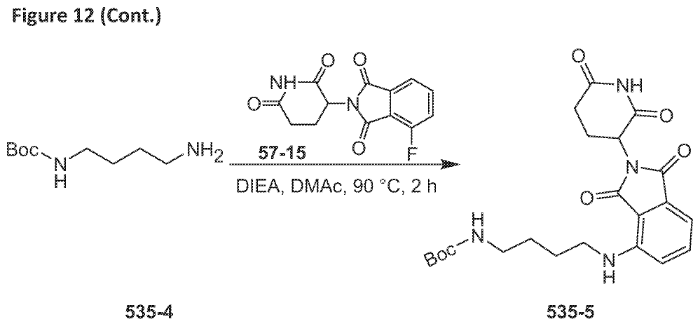

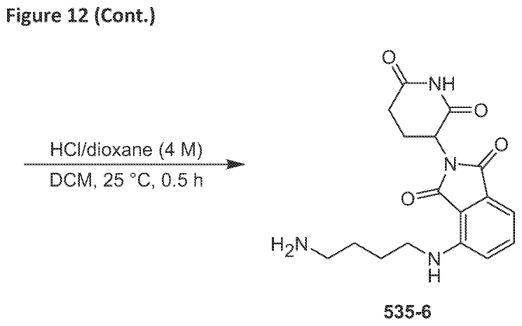

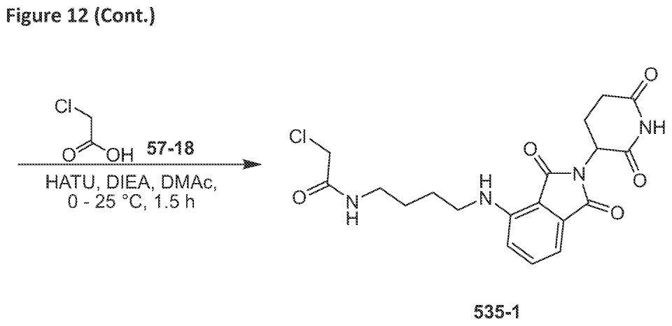

The reaction scheme for the synthesis of MS-5-035 is shown in FIG. 12.

The procedure for preparing compound 56-8 is described in the synthesis of MS-5-006.

Procedure for preparation of compound 58-1. To a solution of compound 535-2 (10 g, 53.69 mmol, 1 eq) and compound 535-3 (12.57 g, 64.43 mmol, 9.24 mL, 1.2 eq) in ACN (50 mL) was added K₂CO₃ (22.26 g, 161.07 mmol, 3 eq). The mixture was stirred at 85° C. for 12 h. The reaction mixture was filtered and the filtrate was concentrated under the reduced pressure to give a residue. The suspension was filtered and the filtrate was concentrated under reduced pressure to give a yellow suspension. The suspension was triturated with ethyl acetate (100 mL) and filtered. The filtrate was concentrated under reduced pressure to give compound 58-1 (16.88 g, crude) as a yellow oil. ¹H NMR (400 MHz, CDCl₃): δ=4.13 (q, J=7.2 Hz, 2H), 3.44-3.35 (m, 4H), 2.45-2.26 (m, 8H), 1.81 (m, 2H), 1.46 (s, 9H), 1.28-1.22 (m, 3H).

Procedure for preparation of compound 57-9. To a solution of compound 58-1 (16.88 g, 56.19 mmol, 1 eq) in EtOH (70 mL) was added NaOH (1 M, 56.19 mL, 1 eq). The mixture was stirred at 50° C. for 1 h. Acidify the reaction mixture by adding, with shaking, 50 mL of HCl (1 M) until pH around 4, then concentrated under the reduced pressure and lyophilized to give compound 57-9 (15.05 g, crude) as a yellow oil. LCMS: t_{R}=0.170 min, MS (ESI+) m/z=273.2 [M+1]⁺. ¹H NMR (400 MHz, DMSO-d₆) δ=3.34-3.22 (m, 4H), 2.33-2.17 (m, 8H), 1.68-1.59 (m, 2H), 1.39 (s, 9H).

Procedure for preparation of compound 57-10. To a solution of compound 56-8 (4.55 g, 16.72 mmol, 1.5 eq) in DCM (50 mL) was added EDCI (3.21 g, 16.72 mmol, 1.5 eq) and TEA (3.38 g, 33.45 mmol, 4.66 mL, 3 eq) and HOBt (1.51 g, 11.15 mmol, 1 eq) at 0° C. for 0.5 h. Then compound 57-9 (5 g, 11.15 mmol, 1 eq) was added and the mixture was stirred at 25° C. for 1 h. The reaction mixture was filtered and the filtrate was concentrated under the reduced pressure to give a residue. The residue was purified by flash silica gel chromatography (SiO₂, DCM/MeOH=100/1 to 10/1) to give compound 57-10 (4.9 g, 6.97 mmol, 62.54% yield) as a black brown solid oil. LCMS: t_{R}=0.367 min, MS (ESI+) m/z=703.3 [M+1]⁺. ¹H NMR (400 MHz, DMSO-d₆) δ=10.36-9.96 (m, 2H), 9.78 (s, 1H), 9.54 (d, J=8.0 Hz, 1H), 8.67 (s, 1H), 7.96 (s, 1H), 7.74-7.65 (m, 1H), 7.65-7.57 (m, 1H), 7.36 (d, J=4.4 Hz, 4H), 7.25 (m, 1H), 5.29 (m, 1H), 5.14 (t, J=4.8 Hz, 1H), 4.10 (q, J=5.2 Hz, 1H), 3.93-3.80 (m, 1H), 3.79-3.68 (m, 1H), 3.31 (s, 1H), 3.17 (d, J=5.2 Hz, 2H), 2.37-2.27 (m, 6H), 2.22 (t, J=7.2 Hz, 2H), 1.72 (m, 2H), 1.57 (s, 3H), 1.53 (s, 3H), 1.39 (s, 9H).

Procedure for preparation of compound 57-11. To a solution of compound 57-10 (4.9 g, 6.97 mmol, 1 eq) in DCM (50 mL) was added TosCl (1.46 g, 7.67 mmol, 1.1 eq) and TEA (1.06 g, 10.46 mmol, 1.46 mL, 1.5 eq). The mixture was stirred at 25° C. for 12 h. The reaction mixture was concentrated under the reduced pressure to give a residue. The residue was purified by prep-HPLC (24%-54% ACN in water (0.05% HCl), 20 min) and lyophilized to give compound 57-11 (3.12 g, 4.56 mmol, 65.35% yield) as a black brown solid. LCMS: t_{R}=0.392 min, MS (ESI+) m/z=685.2 [M+1]⁺. ¹H NMR (400 MHz, DMSO-d₆): δ=10.28 (s, 1H), 8.91 (d, J=8.0 Hz, 1H), 8.66 (s, 1H), 7.97 (s, 1H), 7.75-7.68 (m, 1H), 7.66-7.60 (m, 1H), 7.44-7.31 (m, 4H), 7.29-7.20 (m, 1H), 5.49-5.36 (m, 1H), 5.23 (t, J=4.8 Hz, 1H), 3.97-3.88 (m, 1H), 3.84 (m, 1H), 3.23 (s, 4H), 2.96 (t, J=7.2 Hz, 2H), 2.41 (t, J=6.4 Hz, 2H), 2.34-2.25 (m, 4H), 1.99-1.89 (m, 2H), 1.58 (s, 3H), 1.53 (s, 3H), 1.37 (s, 9H).

Procedure for preparation of compound 57-12. To a solution of compound 57-11 (3 g, 4.38 mmol, 1 eq) in EtOAc (30 mL) was added HCl/EtOAc (4 M, 3.29 mL, 3 eq). The mixture was stirred at 25° C. for 1 h. The reaction mixture was filtered and the filter residue was concentrated under the reduced pressure to give a residue. The residue was purified by prep-HPLC (5%-35% ACN in water (0.1% TFA), 10 min) and lyophilized to give compound 57-12 (1.38 g, 2.22 mmol, 50.71% yield, HCl) as a white solid. LCMS: t_{R}=0.333 min, MS (ESI+) m/z=585.5 [M+1]⁺.

Procedure for preparation of compound 535-5. To a solution of compound 535-4 (5 g, 26.56 mmol, 1 eq) and compound 57-15 (7.34 g, 26.56 mmol, 1 eq) in DMAc (40 mL) was added DIEA (10.30 g, 79.67 mmol, 13.88 mL, 3 eq). The mixture was stirred at 90° C. for 2 h. The residue was purified by prep-HPLC (35%-40% ACN in water (0.05% HCl), 20 min) and lyophilized to give compound 535-5 (3.36 g, 6.35 mmol, 23.91% yield, 84% purity) as a yellow solid. LCMS: t_{R}=0.438 min, MS (ESI+) m/z=344.8 [M+1]⁺. ¹H NMR (400 MHz, DMSO-d₆) δ=11.09 (s, 1H), 7.57 (dd, J=8.4, 7.2 Hz, 1H), 7.10 (d, J=8.8 Hz, 1H), 7.02 (d, J=6.8 Hz, 1H), 6.83 (t, J=5.2 Hz, 1H), 6.55 (t, J=5.6 Hz, 1H), 5.05 (dd, J=12.8, 5.2 Hz, 1H), 3.32-3.27 (m, 2H), 3.00-2.91 (m, 3H), 2.66-2.52 (m, 2H), 2.06-1.96 (m, 1H), 1.55-1.50 (m, 2H), 1.36 (s, 11H).

Procedure for preparation of compound 535-6. To a solution of compound 535-5 (3.3 g, 6.24 mmol, 84% purity, 1 eq) in DCM (33 mL) was added HCl/dioxane (4 M, 4.68 mL, 3 eq). The mixture was stirred at 25° C. for 0.5 h. The reaction mixture was filtered and the filter residue was concentrated under the reduced pressure to give a residue. The residue was purified by prep-HPLC (0%-30% ACN in water (0.05% HCl), 23 min) and lyophilized to give compound 535-6 (870 mg, 2.28 mmol, 36.63% yield, HCl) as a yellow solid. LCMS: t_{R}=0.259 min, MS (ESI+) m/z=344.8 [M+1]⁺. ¹H NMR (400 MHz, DMSO-d₆) δ=11.09 (s, 1H), 7.90 (s, 3H), 7.59 (dd, J=8.4, 7.2 Hz, 1H), 7.13 (d, J=8.4 Hz, 1H), 7.03 (d, J=7.2 Hz, 1H), 6.78-6.40 (m, 1H), 5.05 (dd, J=12.4, 5.2 Hz, 1H), 3.34 (s, 2H), 2.97-2.72 (m, 3H), 2.65-2.53 (m, 2H), 2.12-1.95 (m, 1H), 1.62 (s, 4H).

Procedure for preparation of compound 535-1. To a solution of compound 57-18 (323.82 mg, 3.43 mmol, 385.50 uL, 1.5 eq) in DMAc (10 mL) was added HATU (955.50 mg, 2.51 mmol, 1.1 eq) at 0° C. The mixture was stirred at 0° C. for 1 h. To the cold solution was added compound 535-6 (870 mg, 2.28 mmol, 1 eq, HCl) followed by DIEA (295.26 mg, 2.28 mmol, 397.92 uL, 1 eq). The mixture was stirred at 25° C. for 0.5 h. To the reaction was added brine (30 mL) and ethyl acetate (30 mL) and the layers were separated. The aqueous layer was extracted with ethyl acetate (15 mL×2). The combined organic extracts were washed with brine (30 mL×3), dried, filtered and concentrated. The residue was purified by prep-HPLC (15%-45% ACN in water (0.225% FA), 10 min) and lyophilized to give compound 535-1 (267 mg, 634.44 μmol, 27.77% yield) as a yellow solid. LCMS: t_{R}=0.361 min, MS (ESI+) m/z=421.0 [M+1]⁺. ¹H NMR (400 MHz, DMSO-d₆) δ=11.09 (s, 1H), 8.22 (t, J=5.2 Hz, 1H), 7.58 (dd, J=8.4, 7.2 Hz, 1H), 7.11 (d, J=8.8 Hz, 1H), 7.02 (d, J=6.8 Hz, 1H), 6.56 (s, 1H), 5.05 (dd, J=12.8, 5.6 Hz, 1H), 4.03 (s, 2H), 3.32 (s, 2H), 3.13 (q, J=6.8 Hz, 2H), 2.94-2.82 (m, 1H), 2.63-2.52 (m, 2H), 2.07-1.97 (m, 1H), 1.64-1.43 (m, 4H).

Procedure for preparation of MS-5-035. To a solution of compound 57-12 (100 mg, 161.00 μmol, 1 eq, HCl) and compound 535-1 (67.76 mg, 161.00 μmol, 1 eq) in DMAc (1 mL) was added DIEA (41.62 mg, 321.99 μmol, 56.09 uL, 2 eq). The mixture was stirred at 100° C. for 2 h. The residue was purified by prep-HPLC (19%-49% ACN in water (0.225% FA), 9 min) and lyophilized to give MS-5-035 (53.08 mg, 52.29 μmol, 32.48% yield, 100% purity, FA) as a yellow solid. LCMS: t_{R}=0.406 min, MS (ESI+) m/z=969.4 [M+1]⁺. ¹H NMR (400 MHz, DMSO-d₆) δ=11.09 (s, 1H), 10.27 (s, 1H), 8.91 (d, J=8.0 Hz, 1H), 8.65 (s, 1H), 7.97 (s, 1H), 7.77-7.69 (m, 1H), 7.69-7.61 (m, 2H), 7.57 (dd, J=8.4, 7.2 Hz, 1H), 7.46-7.33 (m, 4H), 7.30-7.22 (m, 1H), 7.09 (d, J=8.8 Hz, 1H), 7.02 (d, J=7.2 Hz, 1H), 6.55 (t, J=6.0 Hz, 1H), 5.49-5.38 (m, 1H), 5.04 (dd, J=12.8, 5.2 Hz, 1H), 3.94-3.88 (m, 1H), 3.87-3.82 (m, 1H), 3.50-3.41 (m, 2H), 3.33-3.28 (m, 2H), 3.16-3.09 (m, 2H), 2.94 (t, J=7.2 Hz, 2H), 2.89-2.83 (m, 1H), 2.81 (s, 2H), 2.62-2.52 (m, 2H), 2.44-2.29 (m, 8H), 2.07-1.98 (m, 1H), 1.92 (m, 2H), 1.60-1.40 (m, 10H).

### Example 23

### Synthesis of MS-5-036

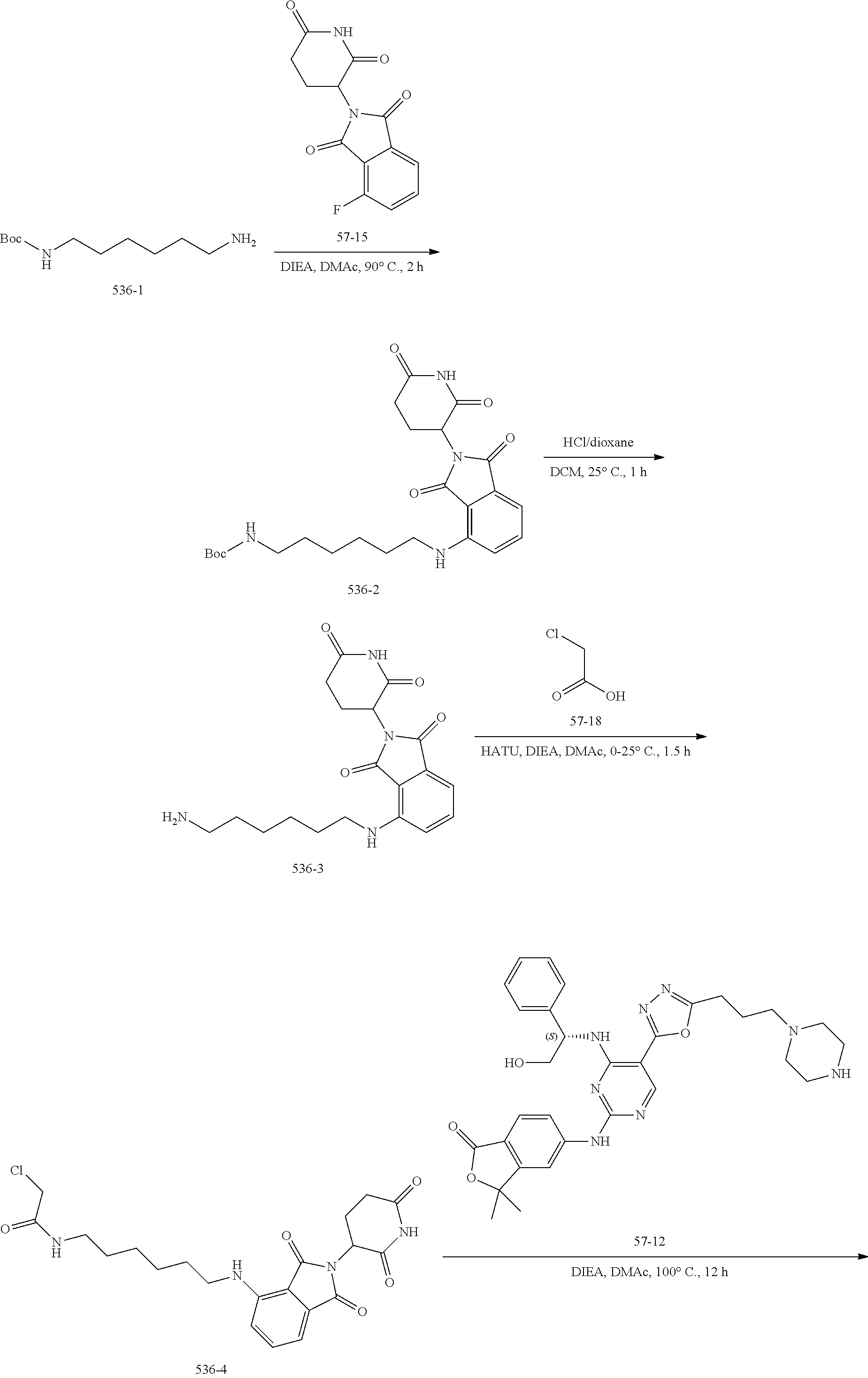

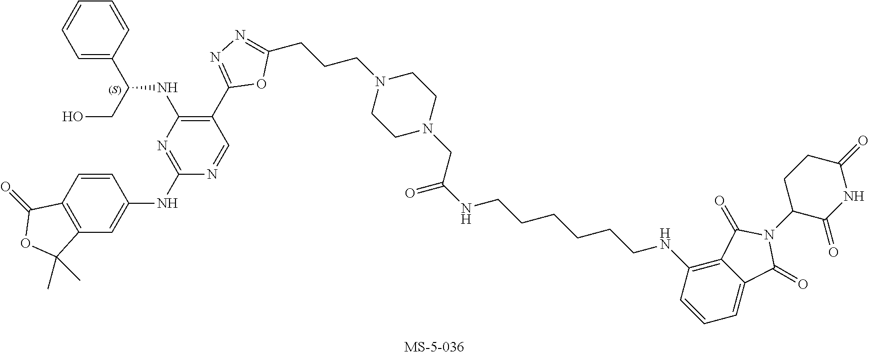

The procedure for preparing compound 57-12 is described in the synthesis of MS-5-007. Procedure for preparation of compound 536-2. To a solution of compound 536-1 (5 g, 23.11 mmol, 1 eq) and compound 57-15 (6.38 g, 23.11 mmol, 1 eq) in DMAc (40 mL) was added DIEA (8.96 g, 69.34 mmol, 12.08 mL, 3 eq). The mixture was stirred at 90° C. for 2 h. The residue was purified by prep-HPLC (25%-45% ACN in water (0.225% FA), 10 min) and lyophilized to give compound 536-2 (4.46 g, 9.44 mmol, 40.83% yield) as a yellow solid. LCMS: t_{R}=0.476 min, MS (ESI+) m/z=373.0 [M+1]⁺. ¹H NMR (400 MHz, DMSO-d₆) δ=11.09 (s, 1H), 7.58 (dd, J=8.4, 7.2 Hz, 1H), 7.09 (d, J=8.8 Hz, 1H), 7.02 (d, J=6.8 Hz, 1H), 6.76 (t, J=5.2 Hz, 1H), 6.53 (t, J=6.0 Hz, 1H), 5.05 (dd, J=13.2, 5.6 Hz, 1H), 3.32-3.25 (m, 2H), 2.94-2.85 (m, 3H), 2.64-2.53 (m, 2H), 2.06-1.97 (m, 1H), 1.57 (t, J=6.8 Hz, 2H), 1.43-1.31 (m, 15H).

Procedure for preparation of compound 536-3. To a solution of compound 536-2 (4.46 g, 9.44 mmol, 1 eq) in DCM (50 mL) was added HCl/dioxane (4 M, 7.08 mL, 3 eq). The mixture was stirred at 25° C. for 1 h. The reaction mixture was filtered and the filter residue was concentrated under the reduced pressure to give compound 536-3 (3.04 g, 7.43 mmol, 78.77% yield, HCl) as a yellow solid. LCMS: t_{R}=0.291 min, MS (ESI+) m/z=372.9 [M+1]⁺. ¹H NMR (400 MHz, DMSO-d₆) δ=11.09 (s, 1H), 7.91 (s, 3H), 7.58 (dd, J=8.4, 7.2 Hz, 1H), 7.10 (d, J=8.8 Hz, 1H), 7.02 (d, J=6.8 Hz, 1H), 6.53 (s, 1H), 5.05 (dd, J=12.8, 5.6 Hz, 1H), 3.30 (t, J=7.2 Hz, 2H), 2.97-2.82 (m, 1H), 2.81-2.69 (m, 2H), 2.63-2.51 (m, 2H), 2.10-1.96 (m, 1H), 1.60-1.51 (m, 4H), 1.35 (s, 4H).

Procedure for preparation of compound 536-4. To a solution of compound 57-18 (1.04 g, 11.01 mmol, 1.24 mL, 1.50 eq) in DMAc (30 mL) was added HATU (3.07 g, 8.07 mmol, 1.1 eq) at 0° C. The mixture was stirred at 0° C. for 1 h. To the cold solution was added compound 536-3 (3 g, 7.34 mmol, 1 eq, HCl) followed by DIEA (948.27 mg, 7.34 mmol, 1.28 mL, 1 eq). The mixture was stirred at 25° C. for 0.5 h. To the reaction was added brine (100 mL) and ethyl acetate (50 mL) and the layers were separated. The aqueous layer was extracted with ethyl acetate (30 mL×2). The combined organic extracts were washed with brine (50 mL×3), dried, filtered and concentrated. The residue was purified by prep-HPLC (TFA condition) and lyophilized to give compound 536-4 (1.14 g, 2.54 mmol, 34.61% yield) as a yellow solid. LCMS: t_{R}=0.414 min, MS (ESI+) m/z=448.8 [M+1]⁺. ¹H NMR (400 MHz, DMSO-d₆) δ=11.01 (s, 1H), 8.17 (t, J=5.2 Hz, 1H), 7.58 (dd, J=8.4, 6.8 Hz, 1H), 7.09 (d, J=8.8 Hz, 1H), 7.02 (d, J=7.2 Hz, 1H), 6.53 (t, J=5.6 Hz, 1H), 5.05 (dd, J=12.8, 7.2 Hz, 1H), 4.02 (s, 2H), 3.31-3.24 (m, 2H), 3.13-3.03 (m, 2H), 2.95-2.81 (m, 1H), 2.63-2.52 (m, 2H), 2.07-1.97 (m, 1H), 1.57 (m, 2H), 1.48-1.39 (m, 2H), 1.37-1.25 (m, 4H).

Procedure for preparation of MS-5-036. To a solution of compound 57-12 (100 mg, 161.00 μmol, 1 eq, HCl) and compound 536-4 (72.27 mg, 161.00 μmol, 1 eq) in DMAc (1 mL) was added DIEA (41.62 mg, 321.99 μmol, 56.09 uL, 2 eq). The mixture was stirred at 100° C. for 12 h. The residue was purified by prep-HPLC (31%-61% ACN in water (10 Mm NH₄HCO₃), 11 min) and lyophilized to give MS-5-036 (24.75 mg, 23.70 μmol, 14.72% yield, 95.5% purity) as a yellow solid. LCMS: t_{R}=0.420 min, MS (ESI+) m/z=997.5 [M+1]⁺. ¹H NMR (400 MHz, DMSO-d₆) δ=11.08 (s, 1H), 10.27 (s, 1H), 8.91 (d, J=8.0 Hz, 1H), 8.65 (s, 1H), 7.97 (s, 1H), 7.76-7.67 (m, 1H), 7.66-7.51 (m, 3H), 7.45-7.32 (m, 4H), 7.31-7.21 (m, 1H), 7.08 (d, J=8.8 Hz, 1H), 7.01 (d, J=6.8 Hz, 1H), 6.52 (t, J=5.6 Hz, 1H), 5.48-5.37 (m, 1H), 5.23 (t, J=4.8 Hz, 1H), 5.04 (dd, J=12.8, 5.6 Hz, 1H), 3.96-3.88 (m, 1H), 3.88-3.79 (m, 1H), 3.29-3.22 (m, 2H), 3.06 (q, J=6.4 Hz, 2H), 2.94 (t, J=7.2 Hz, 2H), 2.89-2.83 (m, 1H), 2.80 (s, 2H), 2.60-2.54 (m, 2H), 2.44-2.23 (m, 10H), 2.06-1.99 (m, 1H), 1.96-1.86 (m, 2H), 1.60-1.49 (m, 7H), 1.46-1.37 (m, 2H), 1.35-1.21 (m, 4H).

### Example 24

### Synthesis of MS-5-037

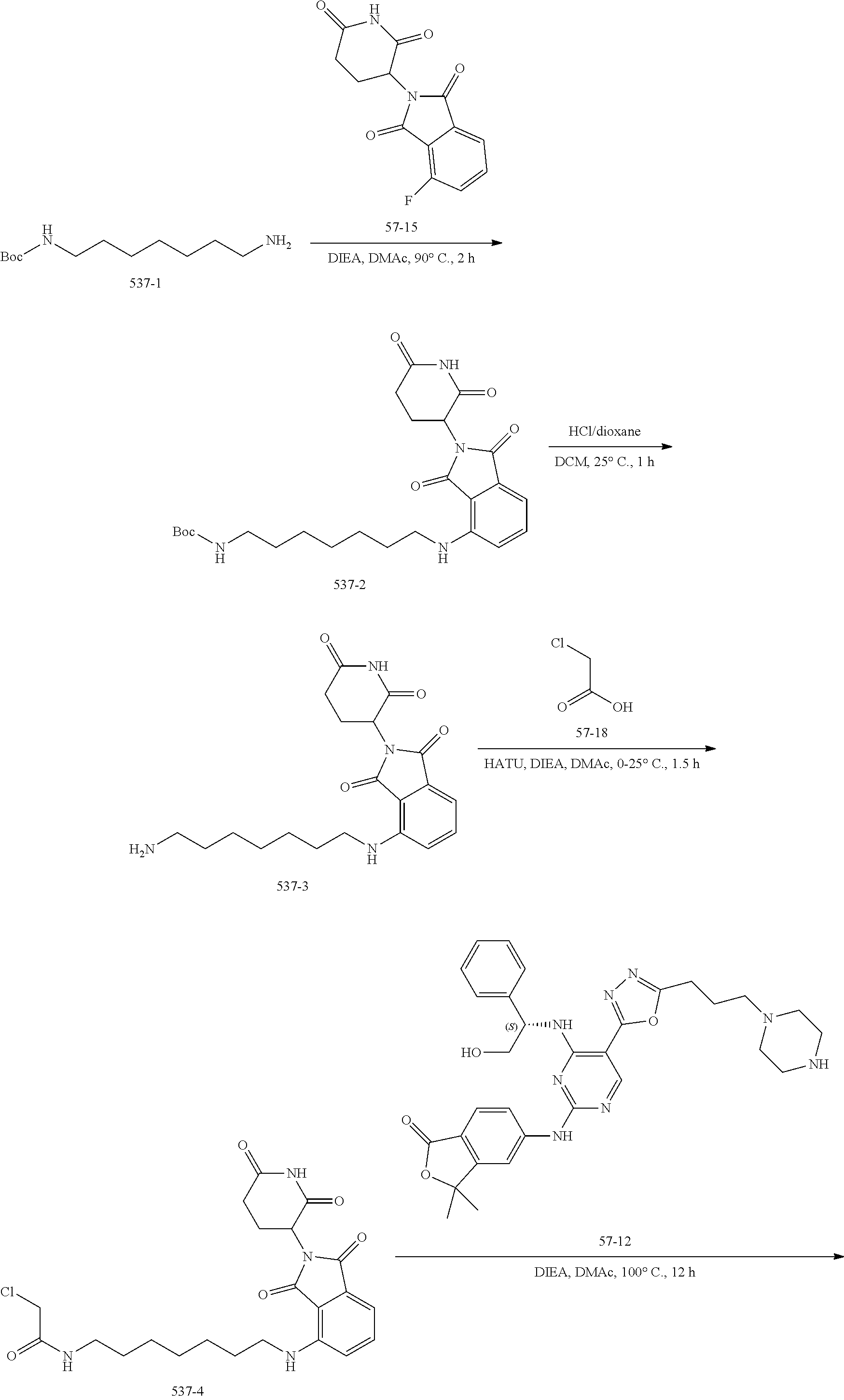

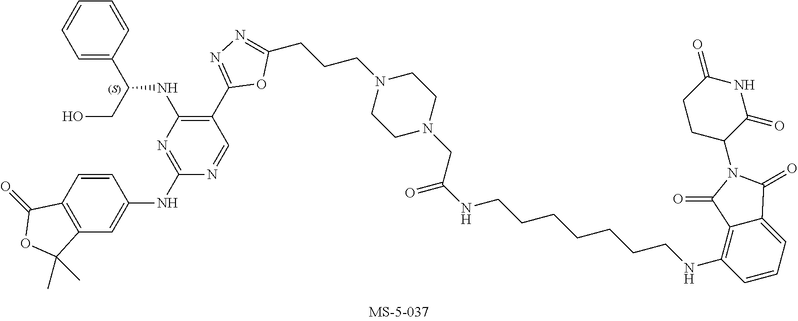

For the procedure for preparing compound 57-12 is described in the synthesis of MS-5-007.

Procedure for preparation of compound 537-2. To a solution of compound 537-1 (1 g, 4.34 mmol, 1 eq) and compound 57-15 (1.20 g, 4.34 mmol, 1 eq) in DMAc (10 mL) was added DIEA (1.68 g, 13.02 mmol, 2.27 mL, 3 eq). The mixture was stirred at 90° C. for 2 h. The residue was purified by prep-HPLC (40%-70% ACN in water (0.225% FA), 20 min) and lyophilized to give compound 537-2 (958 mg, 1.97 mmol, 45.35% yield) as a yellow solid. LCMS: t_{R}=0.497 min, MS (ESI+) m/z=387.0 [M+1]⁺. ¹H NMR (400 MHz, DMSO-d₆) δ=11.09 (s, 1H), 7.58 (dd, J=8.4, 7.2 Hz, 1H), 7.09 (d, J=8.8 Hz, 1H), 7.02 (d, J=6.8 Hz, 1H), 6.76 (t, J=5.2 Hz, 1H), 6.53 (t, J=6.0 Hz, 1H), 5.05 (dd, J=12.8, 5.2 Hz, 1H), 3.31-3.24 (m, 2H), 2.94-2.82 (m, 3H), 2.65-2.52 (m, 2H), 2.12-1.96 (m, 1H), 1.63-1.48 (m, 2H), 1.38-1.23 (m, 17H).

Procedure for preparation of compound 537-3. To a solution of compound 537-2 (950 mg, 1.95 mmol, 1 eq) in DCM (10 mL) was added HCl/dioxane (4 M, 1.46 mL, 3 eq). The mixture was stirred at 25° C. for 1 h. The reaction mixture was filtered and the filter residue was concentrated under the reduced pressure to give compound 537-3 (540 mg, 1.28 mmol, 65.40% yield, HCl) as a yellow solid. LCMS: t_{R}=0.325 min, MS (ESI+) m/z=386.9 [M+1]⁺. ¹H NMR (400 MHz, DMSO-d₆) δ=11.09 (s, 1H), 7.81 (s, 3H), 7.58 (dd, J=8.4, 7.2 Hz, 1H), 7.10 (d, J=8.6 Hz, 1H), 7.03 (d, J=7.2 Hz, 1H), 6.52 (s, 1H), 5.05 (dd, J=12.8, 5.6 Hz, 1H), 3.30 (s, 2H), 2.96-2.81 (m, 1H), 2.81-2.70 (m, 2H), 2.64-2.52 (m, 2H), 2.10-1.95 (m, 1H), 1.61-1.48 (m, 4H), 1.32 (s, 6H).

Procedure for preparation of compound 537-4. To a solution of compound 57-18 (180.99 mg, 1.92 mmol, 215.47 uL, 1.5 eq) in DMAc (6 mL) was added HATU (534.06 mg, 1.40 mmol, 1.1 eq) at 0° C. The mixture was stirred at 0° C. for 1 h. To the cold solution was added compound 537-3 (540 mg, 1.28 mmol, 1 eq, HCl) followed by DIEA (165.03 mg, 1.28 mmol, 222.41 uL, 1 eq). The mixture was stirred at 25° C. for 0.5 h. To the reaction was added brine (30 mL) and ethyl acetate (30 mL) and the layers were separated. The aqueous layer was extracted with ethyl acetate (15 mL×2). The combined organic extracts were washed with brine (30 mL×3), dried, filtered and concentrated. The residue was purified by prep-HPLC (28%-58% ACN in water (0.225% FA), 10 min) and lyophilized to give compound 537-4 (280 mg, 604.85 μmol, 47.37% yield) as a yellow solid. LCMS (EC6212-272-P1B): t_{R}=0.434 min, MS (ESI+) m/z=462.9 [M+1]⁺. ¹H NMR (400 MHz, DMSO-d₆) δ=11.09 (s, 1H), 8.16 (t, J=4.8 Hz, 1H), 7.58 (dd, J=8.4, 7.2 Hz, 1H), 7.09 (d, J=8.8 Hz, 1H), 7.02 (d, J=7.2 Hz, 1H), 6.52 (t, J=5.2 Hz, 1H), 5.05 (dd, J=12.8, 5.2 Hz, 1H), 4.02 (s, 2H), 3.29 (q, J=6.8 Hz, 2H), 3.12-3.01 (m, 2H), 2.96-2.82 (m, 1H), 2.64-2.52 (m, 2H), 2.07-1.97 (m, 1H), 1.64-1.50 (m, 2H), 1.41 (m, 2H), 1.36-1.20 (m, 6H).

Procedure for preparation of MS-5-037. To a solution of compound 57-12 (100 mg, 161.00 μmol, 1 eq, HCl) and compound 537-4 (74.53 mg, 161.00 μmol, 1 eq) in DMAc (1 mL) was added DIEA (41.61 mg, 321.99 μmol, 56.08 uL, 2 eq). The mixture was stirred at 100° C. for 12 h. The residue was purified by prep-HPLC (21%-51% ACN in water (0.225% FA), 10 min) and lyophilized to give MS-5-037 (34.87 mg, 32.98 μmol, 20.49% yield, 100% purity, FA) as a yellow solid. LCMS: t_{R}=0.423 min, MS (ESI+) m/z=1011.4 [M+1]⁺. ¹H NMR (400 MHz, DMSO-d₆) δ=11.09 (s, 1H), 10.27 (s, 1H), 8.91 (d, J=8.0 Hz, 1H), 8.64 (s, 1H), 8.24 (s, 1H), 7.97 (s, 1H), 7.71 (dd, J=8.4, 1.2 Hz, 1H), 7.67-7.51 (m, 3H), 7.44-7.31 (m, 4H), 7.30-7.21 (m, 1H), 7.08 (d, J=8.4 Hz, 1H), 7.01 (d, J=6.8 Hz, 1H), 6.51 (t, J=5.6 Hz, 1H), 5.53-5.38 (m, 1H), 5.38-5.10 (m, 1H), 5.04 (dd, J=12.8, 5.6 Hz, 1H), 3.95-3.88 (m, 1H), 3.87-3.79 (m, 1H), 3.28 (d, J=6.8 Hz, 2H), 3.08-3.02 (m, 2H), 2.94 (t, J=7.2 Hz, 2H), 2.89-2.83 (m, 1H), 2.80 (s, 2H), 2.63-2.52 (m, 2H), 2.47-2.11 (m, 10H), 2.07-1.98 (m, 1H), 1.92 (m, 2H), 1.65-1.46 (m, 7H), 1.43-1.35 (m, 2H), 1.34-1.16 (m, 6H).

### Example 25

### Synthesis of MS-5-038

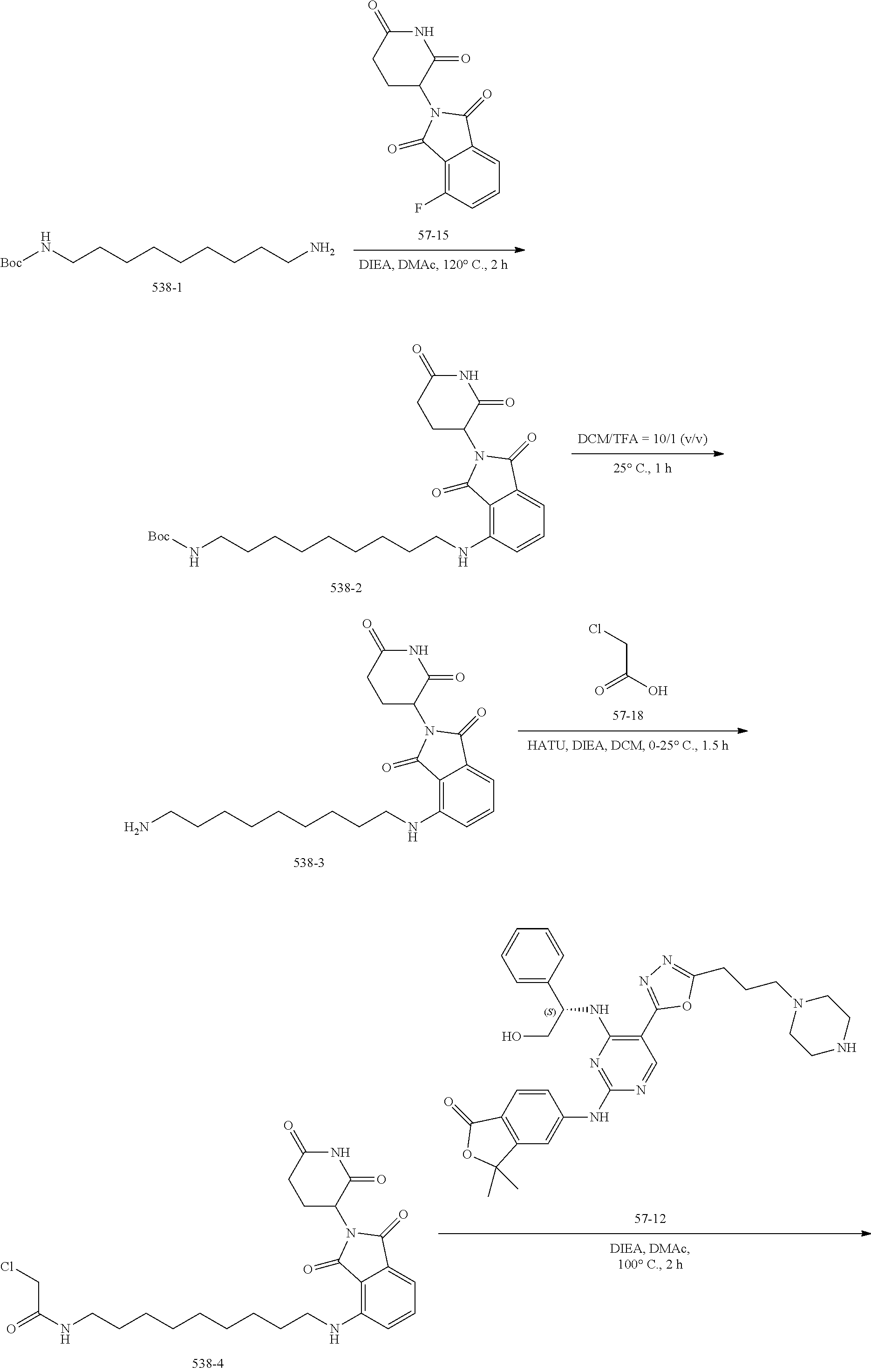

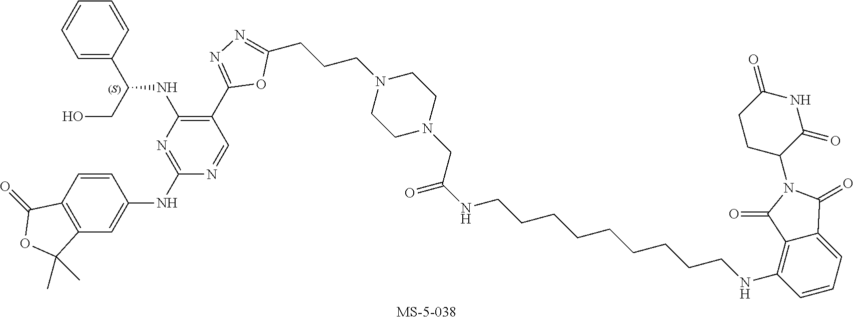

The procedure for preparing compound 57-12 described in the synthesis of MS-5-007.

Procedure for preparation of compound 538-2. To a solution of compound 538-1 (1 g, 3.87 mmol, 1 eq) and compound 57-15 (1.28 g, 4.64 mmol, 1.2 eq) in DMAc (8 mL) was added DIEA (1.50 g, 11.61 mmol, 2.02 mL, 3 eq). The mixture was stirred at 120° C. for 2 h. The residue was purified by prep-HPLC (75%-80% ACN in water (0.225% FA), 20 min) and lyophilized to give compound 538-2 (1.08 g, 2.10 mmol, 54.23% yield) as a yellow solid. LCMS: t_{R}=0.526 min, MS (ESI+) m/z=515.3 [M+1]⁺. ¹H NMR (400 MHz, DMSO-d₆) δ=11.09 (s, 1H), 7.58 (dd, J=8.8, 7.2 Hz, 1H), 7.09 (d, J=8.4 Hz, 1H), 7.01 (d, J=6.8 Hz, 1H), 6.74 (t, J=5.2 Hz, 1H), 6.52 (t, J=6.0 Hz, 1H), 5.05 (dd, J=12.8, 5.2 Hz, 1H), 3.30-3.24 (m, 2H), 2.95-2.80 (m, 3H), 2.64-2.52 (m, 2H), 2.07-1.96 (m, 1H), 1.62-1.48 (m, 2H), 1.40-1.21 (m, 21H).

Procedure for preparation of compound 538-3. To a solution of compound 538-2 (1 g, 1.94 mmol, 1 eq) in DCM (10 mL) was added TFA (1 mL). The mixture was stirred at 25° C. for 1 h. The reaction mixture was concentrated under the reduced pressure to give compound 538-3 (800 mg, crude) as a yellow solid. LCMS: t_{R}=0.370 min, MS (ESI+) m/z=415.2 [M+1]⁺.

Procedure for preparation of compound 538-4. To a solution of compound 57-18 (68.39 mg, 723.77 μmol, 81.42 uL, 1.5 eq) in DCM (3 mL) was added HATU (366.93 mg, 965.03 μmol, 2 eq) at 0° C. The mixture was stirred at 0° C. for 1 h. To the cold solution was added compound 538-3 (200 mg, 482.51 μmol, 1 eq) followed by DIEA (187.08 mg, 1.45 mmol, 252.14 uL, 3 eq). The mixture was stirred at 25° C. for 0.5 h. To the reaction was added brine (10 mL) and ethyl acetate (10 mL) and the layers were separated. The aqueous layer was extracted with ethyl acetate (10 mL×2). The combined organic extracts were washed with brine (30 mL), dried, filtered and concentrated. The residue was purified by prep-HPLC (43%-73% ACN in water (0.225% FA), 10 min) and lyophilized to give compound 538-4 (182 mg, 370.69 μmol, 76.82% yield) as a yellow solid. LCMS: t_{R}=0.467 min, MS (ESI+) m/z=491.1 [M+1]⁺. ¹H NMR (400 MHz, DMSO-d₆) δ=11.08 (s, 1H), 8.23-8.08 (m, 1H), 7.66-7.50 (m, 1H), 7.09 (d, J=8.8 Hz, 1H), 7.02 (d, J=6.8 Hz, 1H), 6.52 (t, J=6.0 Hz, 1H), 5.13-4.94 (m, 1H), 4.01 (s, 2H), 3.30-3.24 (m, 2H), 3.10-3.01 (m, 2H), 2.97-2.80 (m, 1H), 2.65-2.52 (m, 2H), 2.09-1.95 (m, 1H), 1.57 (m, 2H), 1.42-1.17 (m, 12H).

Procedure for preparation of MS-5-038. To a solution of compound 57-12 (100 mg, 193.96 μmol, 1 eq) and compound 538-4 (95.23 mg, 193.96 μmol, 1 eq) in DMAc (1 mL) was added DIEA (50.14 mg, 387.93 μmol, 67.57 uL, 2 eq). The mixture was stirred at 100° C. for 2 h. The residue was purified by prep-HPLC (27%-57% ACN in water (0.225% FA), 10 min) and lyophilized to give MS-5-038 (70.6 mg, 64.21 μmol, 33.10% yield, 98.7% purity, FA) as a yellow solid. LCMS: t_{R}=0.445 min, MS (ESI+) m/z=1039.4 [M+1]⁺. ¹H NMR (400 MHz, DMSO-d₆) δ=11.10 (br s, 1H), 10.27 (s, 1H), 8.90 (d, J=7.6 Hz, 1H), 8.64 (s, 1H), 8.18 (s, 1H), 7.96 (s, 1H), 7.71 (d, J=8.8 Hz, 1H), 7.67-7.50 (m, 3H), 7.46-7.32 (m, 4H), 7.30-7.21 (m, 1H), 7.07 (d, J=8.8 Hz, 1H), 7.00 (d, J=7.2 Hz, 1H), 6.51 (t, J=5.6 Hz, 1H), 5.43 (dd, J=4.8, 1.6 Hz, 1H), 5.31-5.16 (m, 1H), 5.04 (dd, J=12.8, 5.6 Hz, 1H), 3.95-3.88 (m, 1H), 3.84 (dd, J=13.6, 5.2 Hz, 1H), 3.12-3.00 (m, 4H), 2.94 (t, J=67.2 Hz, 2H), 2.89-2.84 (m, 1H), 2.80 (s, 2H), 2.60-2.55 (m, 2H), 2.44-2.29 (m, 10H), 2.07-1.99 (m, 1H), 1.97-1.86 (m, 2H), 1.64-1.47 (m, 8H), 1.41-1.19 (m, 12H).

### Example 26

### Synthesis of MS-5-039

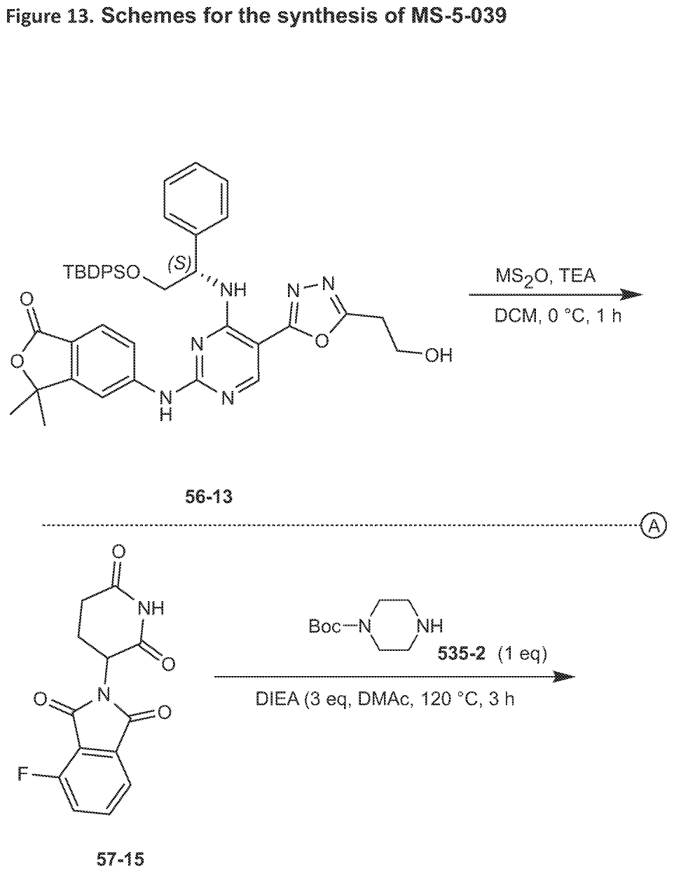

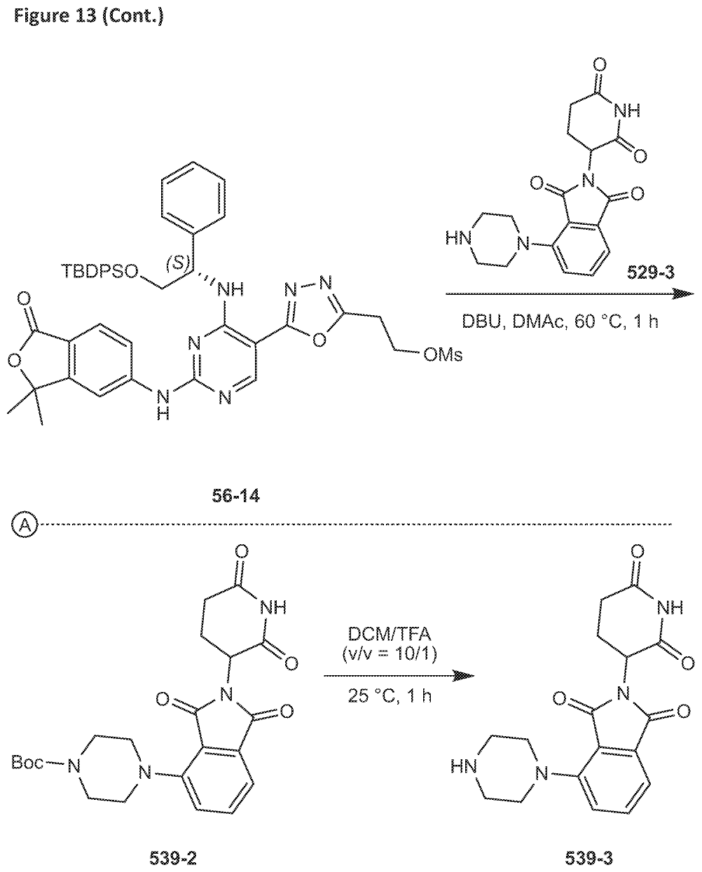

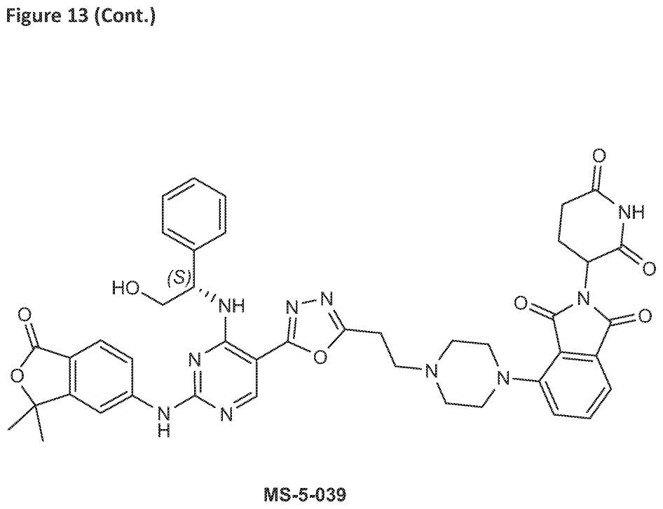

The reaction scheme for the synthesis of MS-5-039 is shown in FIG. 13.

Procedure for preparation of compound 539-2. To a solution of compound 57-15 (2 g, 7.24 mmol, 1.2 eq) and compound 535-2 (1.12 g, 6.03 mmol, 1 eq) in DMAc (15 mL) was added DIEA (2.34 g, 18.10 mmol, 3.15 mL, 3 eq). The mixture was stirred at 120° C. for 3 h. The reaction mixture was filtered and the filtrate was concentrated under the reduced pressure. The residue was purified by prep-HPLC (1%-30% ACN in water (0.225% FA), 8 min) and lyophilized to give the compound 539-2 (2.05 g, 4.20 mmol, 90% yield) as a yellow solid. LCMS: t_{R}=0.437 min, MS (ESI+) m/z=442.19[M+1]⁺. ¹H NMR (400 MHz, DMSO-d₆): δ=11.09 (s, 1H), 7.72 (dd, J=8.4, 7.2 Hz, 1H), 7.37 (dd, J=16.4, 8.0 Hz, 2H), 5.10 (dd, J=12.8, 4.8 Hz, 1H), 3.51 (s, 4H), 3.25 (d, J=4.0 Hz, 4H), 2.96-2.82 (m, 1H), 2.63-2.56 (m, 2H), 2.09-1.97 (m, 1H), 1.42 (s, 9H).

Procedure for preparation of compound 539-3. To a solution of compound 539-2 (2 g, 4.52 mmol, 1 eq) in DCM (20 mL) was added TFA (2 mL). The mixture was stirred at 25° C. for 1 h. The reaction mixture was filtered and the filtrate was concentrated under the reduced pressure. The residue was purified by prep-HPLC (0%-30% ACN in water (0.225% FA), 23 min) and lyophilized to give the compound 539-3 (1.34 g, 3.91 mmol, 86.59% yield) as a yellow solid. LCMS: t_{R}=0.425 min, MS (ESI+) m/z=342.13[M+1]⁺. ¹H NMR (400 MHz, DMSO-d₆): δ=10.11 (s, 1H), 7.94 (s, 2H), 6.83-6.72 (m, 1H), 6.53-6.38 (m, 2H), 4.13 (dd, J=12.8, 5.2 Hz, 1H), 2.50 (s, 4H), 2.32 (s, 4H), 1.98-1.82 (m, 1H), 1.67-1.53 (m, 2H), 1.12-0.99 (m, 1H).

Procedure for preparation of compound 56-14. To a solution of compound 56-13 (1 g, 1.35 mmol, 1 eq) in DCM (10 mL) was added TEA (682.86 mg, 6.75 mmol, 939.29 μL, 5 eq) and methylsulfonyl methanesulfonate (705.32 mg, 4.05 mmol, 3 eq). The mixture was stirred at 0° C. for 1 h. The reaction mixture was filtered and the filtrate was concentrated under the reduced pressure to give the compound 56-14 (1.5 g, 1.83 mmol, 90% yield) as a yellow oil. LCMS: t_{R}=0.597 min, MS (ESI+) m/z=819.01[M+1]⁺.

Procedure for preparation of MS-5-039. To a solution of compound 56-14 (300 mg, 876.30 μmol, 1.5 eq) in DMAc (3 mL) was added dropwise DBU (444.68 mg, 2.92 mmol, 440.28 μL, 5 eq) at 80° C. After addition, the mixture was stirred at 80° C. for 5 min, then compound 539-3 (478.47 mg, 584.20 μmol, 1 eq) was added dropwise at 80° C. The resulting mixture was stirred at 80° C. for 60 min. The reaction mixture was filtered and the filtrate was concentrated under the reduced pressure. The residue was purified by prep-HPLC (30%-60% ACN in water (10 mM NH₄HCO₃), 10 min) to give the MS-5-039 (0.017 g, 16.62 mmol, 100% yield) as a yellow solid. LCMS: t_{R}=0.382 min, MS (ESI+) m/z=1064.44[M+1]⁺. ¹H NMR (400 MHz, DMSO-d₆): δ=11.09 (s, 1H), 10.30 (s, 1H), 8.89 (d, J=7.4 Hz, 1H), 8.67 (s, 1H), 7.98 (s, 1H), 7.77-7.60 (m, 3H), 7.44-7.23 (m, 7H), 5.50-5.38 (m, 1H), 5.25 (t, J=4.8 Hz, 1H), 5.09 (dd, J=12.4, 5.2 Hz, 1H), 3.96-3.89 (m, 1H), 3.87-3.79 (m, 1H), 3.56-3.38 (m, 2H), 3.30 (s, 2H), 3.19 (t, J=6.8 Hz, 2H), 2.93-2.81 (m, 3H), 2.72-2.64 (m, 4H), 2.62-2.56 (m, 2H), 2.10-1.97 (m, 1H), 1.58 (s, 3H), 1.53 (s, 3H).

### Example 27

### Synthesis of MS-5-040

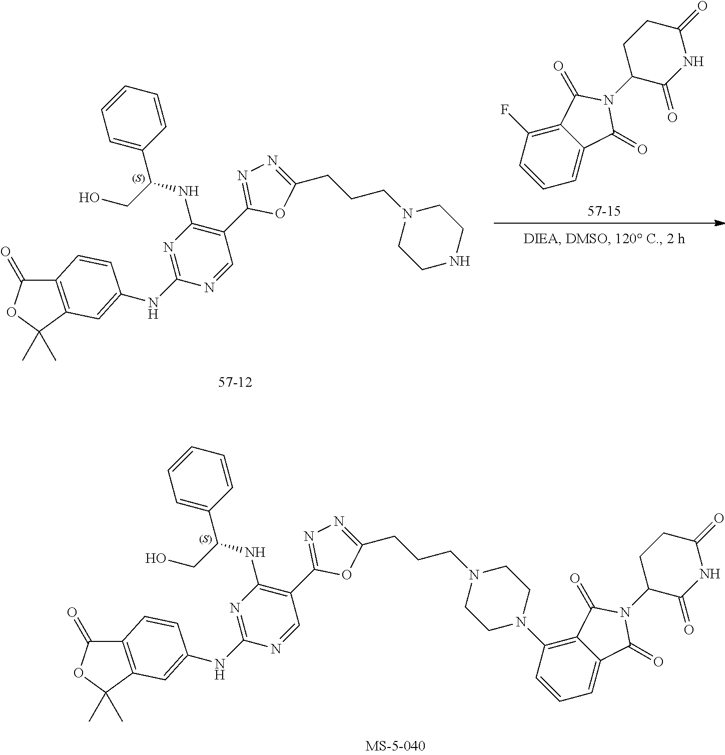

The procedure for preparing compound 57-12 is described in the synthesis of MS-5-007.

Procedure for preparation of MS-5-040. To a solution of compound 57-12 (60 mg, 102.62 μmol, 1 eq) and compound 57-15 (34.02 mg, 123.15 μmol, 1.2 eq) in DMSO (1 mL) was added DIEA (39.79 mg, 307.87 μmol, 53.62 uL, 3 eq). The mixture was stirred at 120° C. for 2 h. The residue was purified by prep-HPLC (water(FA)-ACN]; B %: 18%-48%, 10 min) and by prep-HPLC (32%-62% ACN in water (10 mM NH₄HCO₃), 10 min) and lyophilized to give MS-5-040 (29.95 mg, 35.26 μmol, 34.36% yield, 99% purity) as a yellow solid. LCMS: t_{R}=0.628 min, MS (ESI+) m/z=841.6 [M+1]⁺. ¹H NMR (400 MHz, DMSO-d₆) δ=11.08 (s, 1H), 10.28 (s, 1H), 8.91 (d, J=8.0 Hz, 1H), 8.67 (s, 1H), 7.97 (s, 1H), 7.75-7.68 (m, 1H), 7.67-7.59 (m, 2H), 7.42-7.29 (m, 5H), 7.28-7.19 (m, 2H), 5.48-5.35 (m, 1H), 5.23 (t, J=4.8 Hz, 1H), 5.08 (dd, J=12.8, 2.4 Hz, 1H), 3.94-3.86 (m, 1H), 3.86-3.78 (m, 1H), 3.22 (br s, 4H), 3.00 (t, J=Hz, 2H), 2.93-2.78 (m, 1H), 2.63-2.52 (m, 6H), 2.47 (br s, 2H), 2.05-1.96 (m, 3H), 1.58 (s, 3H), 1.53 (s, 3H).

### Example 28

### Synthesis of MS-5-041

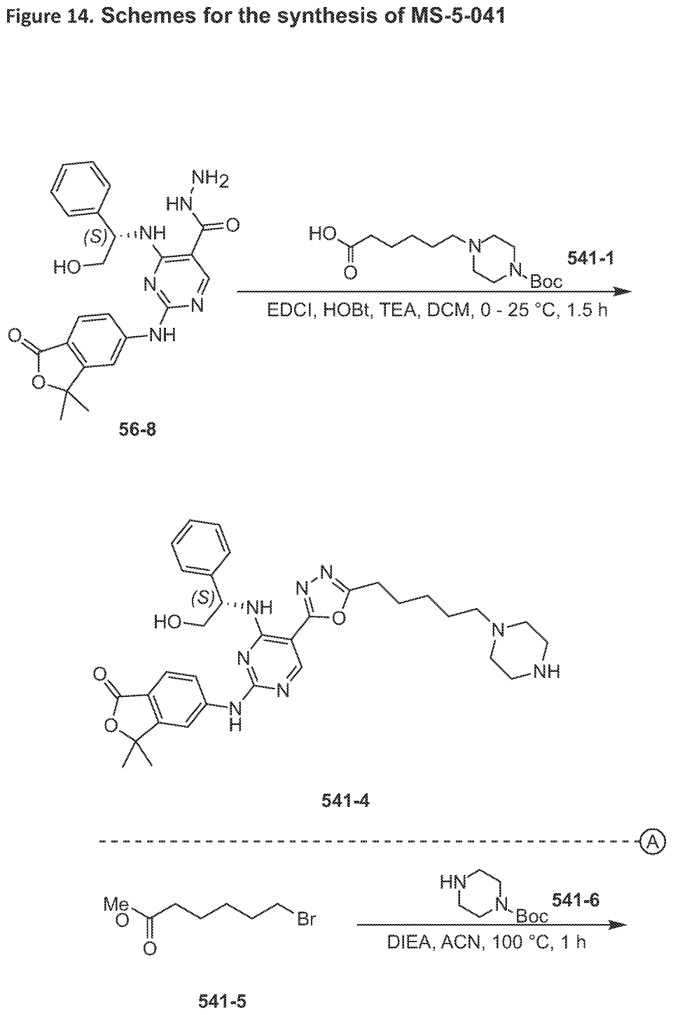

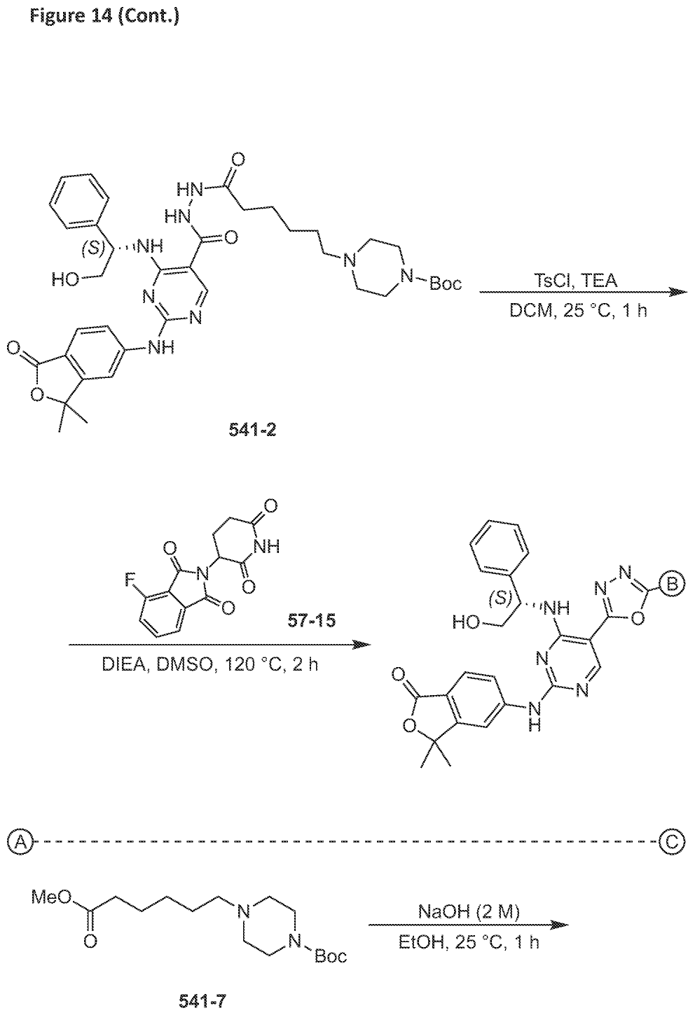

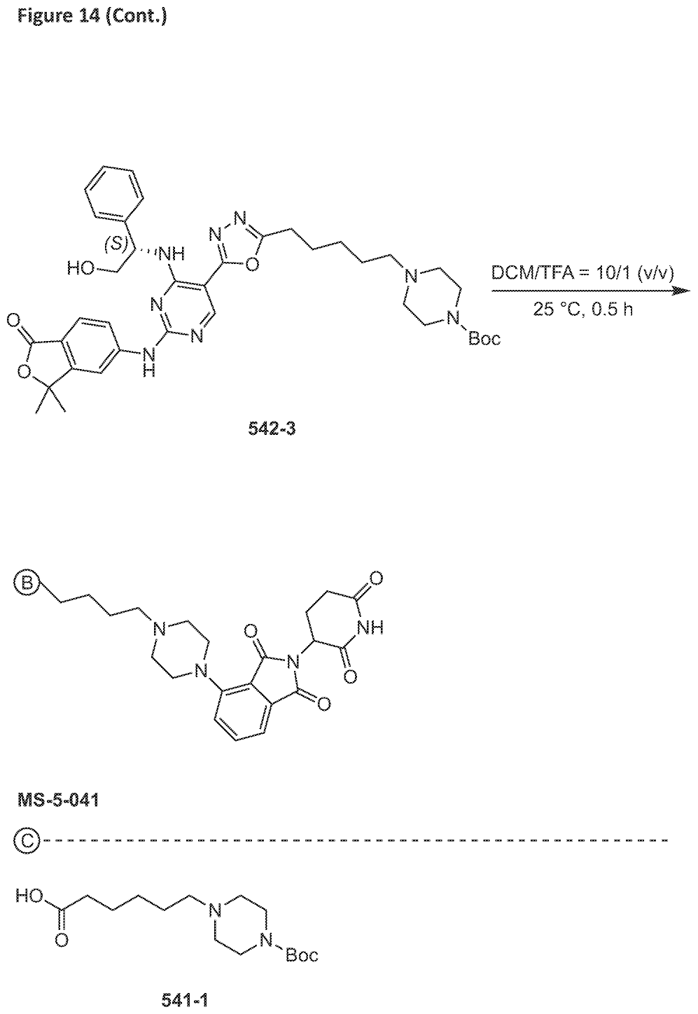

The reaction scheme for the synthesis of MS-5-041 is shown in FIG. 14.

Procedure for preparation of compound 541-7. To a solution of compound 541-5 (5 g, 23.91 mmol, 1 eq) and compound 541-6 (4.45 g, 23.91 mmol, 1 eq) in ACN (50 mL) was added DIEA (6.18 g, 47.83 mmol, 8.33 mL, 2 eq). The mixture was stirred at 100° C. for 1 h. The reaction mixture were washed with citric acid (50 mL), and extracted with EtOAc (50 mL×3). The combined organic layers were dried over anhydrous Na₂SO₄, filtered and concentrated under reduced pressure to give compound 541-7 (5.2 g, crude) as a brown solid. LCMS: t_{R}=0.266 min, MS (ESI+) m/z=315.2 [M+1]⁺. ¹H NMR (400 MHz, DMSO-d₆) δ=3.58 (s, 3H), 3.32 (s, 4H), 2.68-2.43 (m, 6H), 2.31 (t, J=7.2 Hz, 2H), 1.63-1.46 (m, 4H), 1.40 (s, 9H), 1.31-1.23 (m, 2H).

Procedure for preparation of compound 541-1. To a solution of compound 541-7 (5.2 g, 16.54 mmol, 1 eq) in EtOH (50 mL) was added NaOH (2 M, 8.27 mL, 1 eq). The mixture was stirred at 25° C. for 1 h. Acidify the reaction mixture by adding, with shaking, 20 mL of citric acid until pH around 6, and then extracted with EtOAc (50 mL×2). The combined organic layers were dried over anhydrous Na₂SO₄, filtered and concentrated to give compound 541-1 (1.46 g, 4.86 mmol, 29.39% yield) as a yellow oil. LCMS: t_{R}=0.230 min, MS (ESI+) m/z=301.2 [M+1]⁺. ¹H NMR (400 MHz, CDCl₃) δ=3.57-3.48 (m, 4H), 2.60 (t, J=4.8 Hz, 4H), 2.54-2.46 (m, 2H), 2.29 (t, J=7.2 Hz, 2H), 1.61 (m, 4H), 1.46 (s, 8H), 1.41-1.35 (m, 2H).

Procedure for preparation of compound 541-2. To a solution of compound 541-1 (1.31 g, 4.35 mmol, 1.5 eq) in DCM (13 mL) was added EDCI (833.54 mg, 4.35 mmol, 1.5 eq), HOBt (391.68 mg, 2.90 mmol, 1 eq) and TEA (879.96 mg, 8.70 mmol, 1.21 mL, 3 eq) at 0° C. for 0.5 h. Then compound 56-8 (1.3 g, 2.90 mmol, 1 eq) was added and the mixture was stirred at 25° C. for 1 h. The reaction mixture were washed with water (20 mL), and extracted with DCM (20 mL×2). The combined organic layers were dried over anhydrous Na₂SO₄, filtered and concentrated under reduced pressure to give compound 541-2 (2.6 g, crude) as a yellow solid. LCMS: t_{R}=0.367 min, MS (ESI+) m/z=731.4 [M+1]⁺.

Procedure for preparation of compound 541-3. To a solution of compound 541-2 (2.6 g, 3.56 mmol, 1 eq) in DCM (20 mL) was added TosCl (746.05 mg, 3.91 mmol, 1.1 eq) and TEA (539.97 mg, 5.34 mmol, 742.74 uL, 1.5 eq). The mixture was stirred at 25° C. for 1 h. The reaction mixture was partitioned between EtOAc (50 mL) and water (50 mL). After quenching the reaction, the reaction mixture was poured into separatory funnel and separated. The residue was purified by prep-HPLC (25%-60% ACN in water (0.225% FA), 18 min) and lyophilized to give compound 541-3 (831 mg, 1.17 mmol, 32.77% yield) as a white solid. LCMS: t_{R}=0.402 min, MS (ESI+) m/z=713.4 [M+1]⁺. ¹H NMR (400 MHz, DMSO-d₆) δ=10.28 (s, 1H), 8.88 (d, J=7.6 Hz, 1H), 8.64 (s, 1H), 7.97 (d, J=1.2 Hz, 1H), 7.75-7.68 (m, 1H), 7.66-7.60 (m, 1H), 7.44-7.31 (m, 4H), 7.30-7.22 (m, 1H), 5.49-5.38 (m, 1H), 5.23 (s, 1H), 3.97-3.88 (m, 1H), 3.88-3.78 (m, 1H), 2.95 (t, J=7.2 Hz, 2H), 2.66-2.51 (m, 4H), 2.49 (s, 4H), 1.89-1.72 (m, 2H), 1.68-1.26 (m, 20H).

Procedure for preparation of compound 541-4. To a solution of compound 541-3 (200 mg, 280.57 μmol, 1 eq) in TFA (0.2 mL) was added DCM (2 mL). The mixture was stirred at 25° C. for 0.5 h. Acidify the reaction mixture by adding, with shaking, 1 mL of NH₃H₂O until pH around 9, and then extracted with DCM (2 mL×2). The combined organic layers were dried over anhydrous Na₂SO₄, filtered and concentrated to give compound 541-4 (200 mg, crude) as a yellow oil. LCMS: t_{R}=0.344 min, MS (ESI+) m/z=613.3 [M+1]⁺ Procedure for preparation of MS-5-041. To a solution of compound 541-4 (100 mg, 163.21 μmol, 1 eq) and compound 57-15 (45.08 mg, 163.21 μmol, 1 eq) in DMSO (2 mL) was added DIEA (42.19 mg, 326.41 μmol, 56.86 uL, 2 eq). The mixture was stirred at 120° C. for 2 h. The reaction mixture was purified by prep-HPLC (32%-62% ACN in water (10 mM NH₄HCO₃), 8 min) then prep-HPLC (25%-45% ACN in water (0.225% FA), 10 min) and lyophilized to give MS-5-041 (24.61 mg, 26.09 μmol, 15.99% yield, 97% purity, FA) as a yellow solid. LCMS: t_{R}=0.387 min, MS (ESI+) m/z=869.4 [M+1]⁺. ¹H NMR (400 MHz, DMSO-d₆) δ=11.08 (s, 1H), 10.28 (s, 1H), 8.89 (d, J=8.0 Hz, 1H), 8.65 (s, 1H), 8.18 (s, 1H), 7.96 (s, 1H), 7.75-7.68 (m, 1H), 7.67-7.59 (m, 2H), 7.45-7.14 (m, 7H), 5.51-5.37 (m, 1H), 5.23 (s, 1H), 5.08 (dd, J=12.8, 5.2 Hz, 1H), 3.98-3.87 (m, 1H), 3.87-3.77 (m, 1H), 3.27 (s, 4H), 2.96 (t, J=7.2 Hz, 2H), 2.92-2.83 (m, 1H), 2.64-2.51 (m, 6H), 2.36 (t, J=6.8 Hz, 2H), 2.06-1.94 (m, 1H), 1.82 (m, 2H), 1.60-1.36 (m, 10H).

### Example 29

### Synthesis of MS-5-042

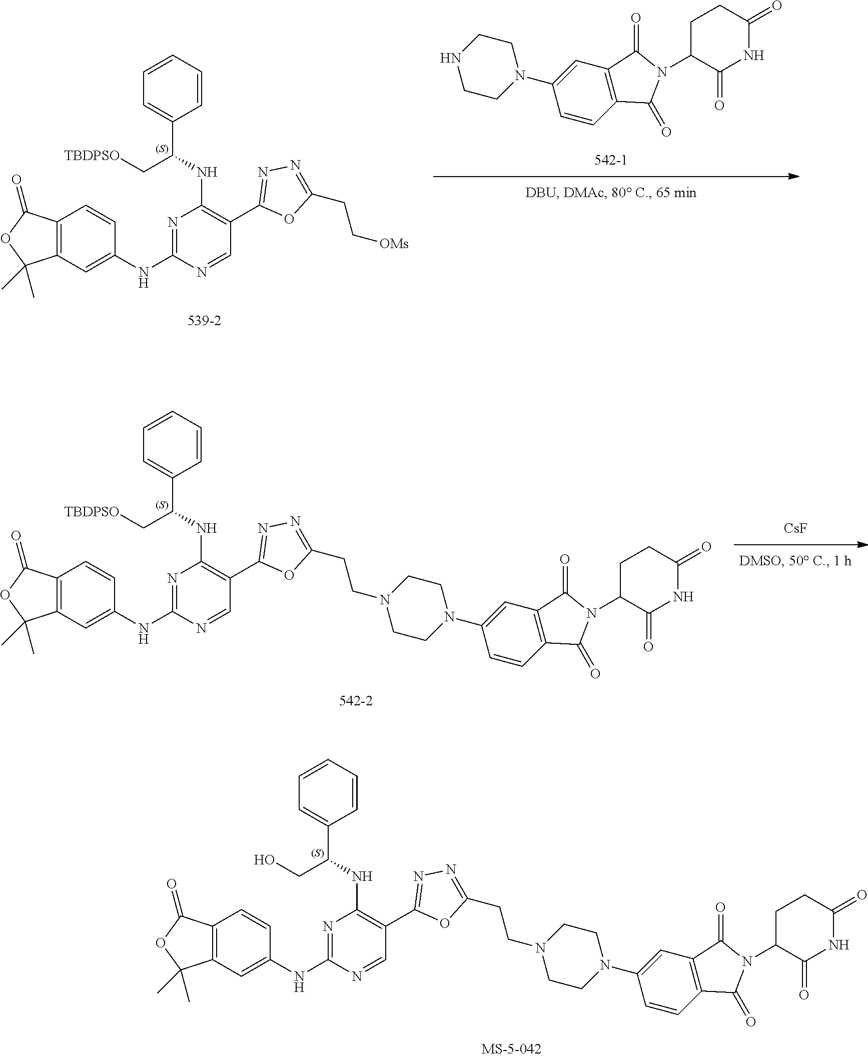

Procedure for preparation of compound 542-2. To a solution of compound 539-2 (188.10 mg, 549.44 μmol, 1.5 eq) in DMAc (3 mL) was added dropwise DBU (278.82 mg, 1.83 mmol, 276.06 L, 5 eq) at 80° C. After addition, the mixture was stirred at 80° C. for 5 min, then compound 542-1 (300 mg, 366.30 μmol, 1 eq) was added dropwise at 80° C. The resulting mixture was stirred at 80° C. for 1 h. The reaction was monitored by LCMS, LCMS showed compound 539-2 was consumed and the desired mass was detected. The residue was purified by prep-HPLC (45%-75% ACN in water (0.225% FA), 10 min) and lyophilized to give compound 542-2 (65 mg, 61.02 μmol, 16.66% yield) as a yellow solid. LCMS: t_{R}=0.512 min, MS (ESI+) m/z=1064.44[M+1]⁺ Procedure for preparation of MS-5-042. To a solution of compound 542-2 (60 mg, 56.32 μmol, 1 eq) in DMSO (1 mL) was added CsF (85.56 mg, 563.25 μmol, 20.77 μL, 10 eq). The mixture was stirred at 50° C. for 1 h. The reaction was monitored by LCMS, LCMS showed compound 542-2 was consumed and the desired mass was detected. The residue was purified by prep-HPLC (17%-47% ACN in water (0.225% FA), 10 min) and lyophilized to give the product to give MS-5-042 (18.45 mg, 22.31 μmol, 39.62% yield, 100% purity) as a yellow solid. LCMS: t_{R}=0.382 min, MS (ESI+) m/z=826.32[M+1]⁺. ¹H NMR (400 MHz, DMSO-d₆) δ=11.08 (s, 1H), 10.29 (s, 1H), 8.88 (d, J=8 Hz, 1H), 8.67 (s, 1H), 8.13 (s, 1H), 7.97 (s, 1H), 7.79-7.57 (m, 3H), 7.38 (d, J=14.8 Hz, 5H), 7.31-7.20 (m, 2H), 5.51-5.35 (m, 1H), 5.24 (t, J=4.8 Hz, 1H), 5.07 (dd, J=12.4, 5.2 Hz, 1H), 3.97-3.88 (m, 1H), 3.88-3.78 (m, 1H), 3.44 (s, 4H), 3.19 (s, 2H), 2.95-2.81 (m, 3H), 2.63 (s, 4H), 2.58-2.53 (m, 2H), 2.06-1.95 (m, 1H), 1.58 (s, 3H), 1.53 (s, 3H).

### Example 30

### Synthesis of MS-5-043

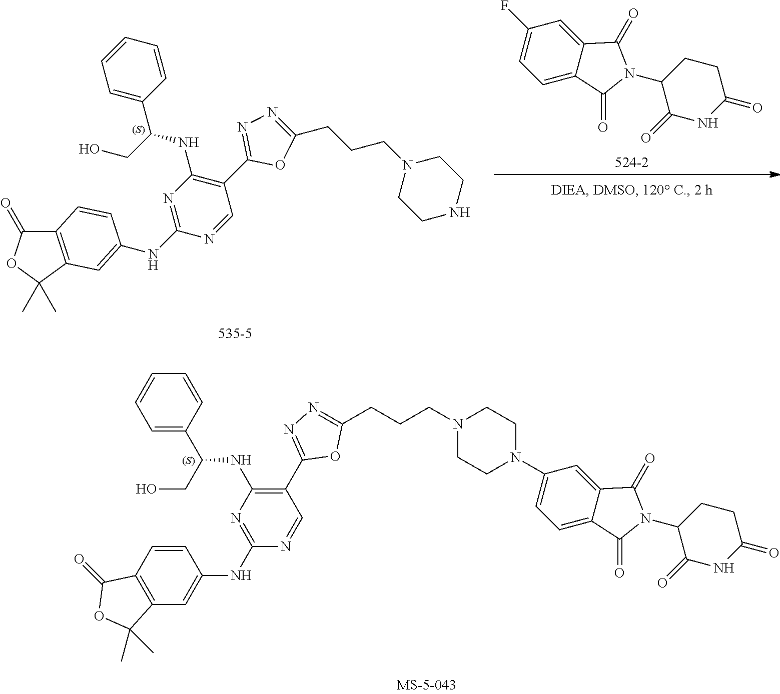

Procedure for preparation of MS-5-043. To a solution of compound 535-5 (60 mg, 102.62 μmol, 1 eq) and compound 524-2 (32.02 mg, 115.92 μmol, 1.2 eq) in DMSO (1 mL) was added DIEA (37.45 mg, 289.80 μmol, 50.48 uL, 3 eq). The mixture was stirred at 120° C. for 2 h. The residue was purified by prep-HPLC (11%-41% ACN in water (0.05% HCl), 8 min) and prep-HPLC (32%-62% ACN in water (10 mM NH₄HCO₃), 10 min) and lyophilized to give MS-5-043 (13.01 mg, 15.16 μmol, 15.70% yield, 98% purity) as a yellow solid. LCMS: t_{R}=0.386 min, MS (ESI+) m/z=841.4 [M+1]⁺. ¹H NMR (400 MHz, DMSO-d₆) δ=11.07 (s, 1H), 10.27 (s, 1H), 8.90 (d, J=7.6 Hz, 1H), 8.67 (s, 1H), 7.95 (s, 1H), 7.73-7.67 (m, 1H), 7.66-7.60 (m, 2H), 7.42-7.29 (m, 5H), 7.23 (dd, J=15.6, 7.2 Hz, 2H), 5.49-5.33 (m, 1H), 5.22 (t, J=3.6 Hz, 1H), 5.06 (dd, J=12.8, 5.2 Hz, 1H), 3.93-3.86 (m, 1H), 3.85-3.77 (m, 1H), 3.37 (d, J=4.8 Hz, 4H), 3.05-2.95 (m, 2H), 2.94-2.82 (m, 1H), 2.63-2.55 (m, 2H), 2.49-2.43 (m, 6H), 2.04-1.95 (m, 3H), 1.58 (s, 3H), 1.53 (s, 3H).

### Example 31

### Synthesis of MS-5-044

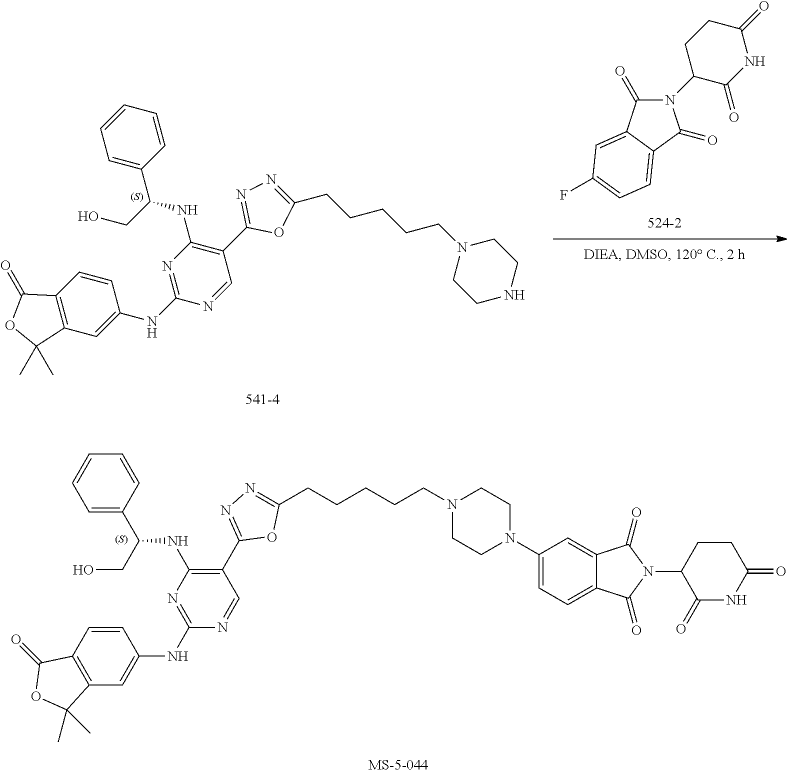

Procedure for preparation of MS-5-044. To a solution of compound 541-4 (90 mg, 146.89 μmol, 1 eq) and compound 524-2 (40.57 mg, 146.89 μmol, 1 eq) in DMSO (1 mL) was added DIEA (37.97 mg, 293.77 μmol, 51.17 uL, 2 eq). The mixture was stirred at 120° C. for 2 h. The residue was purified by prep-HPLC (33%-63% ACN in water (10 mM NH₄HCO₃), 8 min) and prep-HPLC (25%-45% ACN in water (0.225% FA), 10 min) and lyophilized to give MS-5-044 (30.13 mg, 31.61 μmol, 21.52% yield, 96% purity, FA) as a yellow solid. LCMS: t_{R}=0.378 min, MS (ESI+) m/z=869.5 [M+1]⁺. ¹H NMR (400 MHz, DMSO-d₆) δ=11.07 (s, 1H), 10.27 (s, 1H), 8.89 (d, J=7.6 Hz, 1H), 8.65 (s, 1H), 8.16 (s, 1H), 7.96 (s, 1H), 7.75-7.67 (m, 1H), 7.63 (d, J=8.4 Hz, 2H), 7.45-7.29 (m, 5H), 7.29-7.16 (m, 2H), 5.51-5.35 (m, 1H), 5.24 (s, 1H), 5.06 (dd, J=12.4, 5.2 Hz, 1H), 3.97-3.88 (m, 1H), 3.87-3.79 (m, 1H), 3.40 (d, J=4.4 Hz, 4H), 2.95 (t, J=7.6 Hz, 2H), 2.91-2.81 (m, 1H), 2.64-2.52 (m, 2H), 2.34 (t, J=6.8 Hz, 6H), 2.06-1.95 (m, 1H), 1.82 (m, 2H), 1.61-1.41 (m, 10H).

### Example 32

### Synthesis of MS-5-045

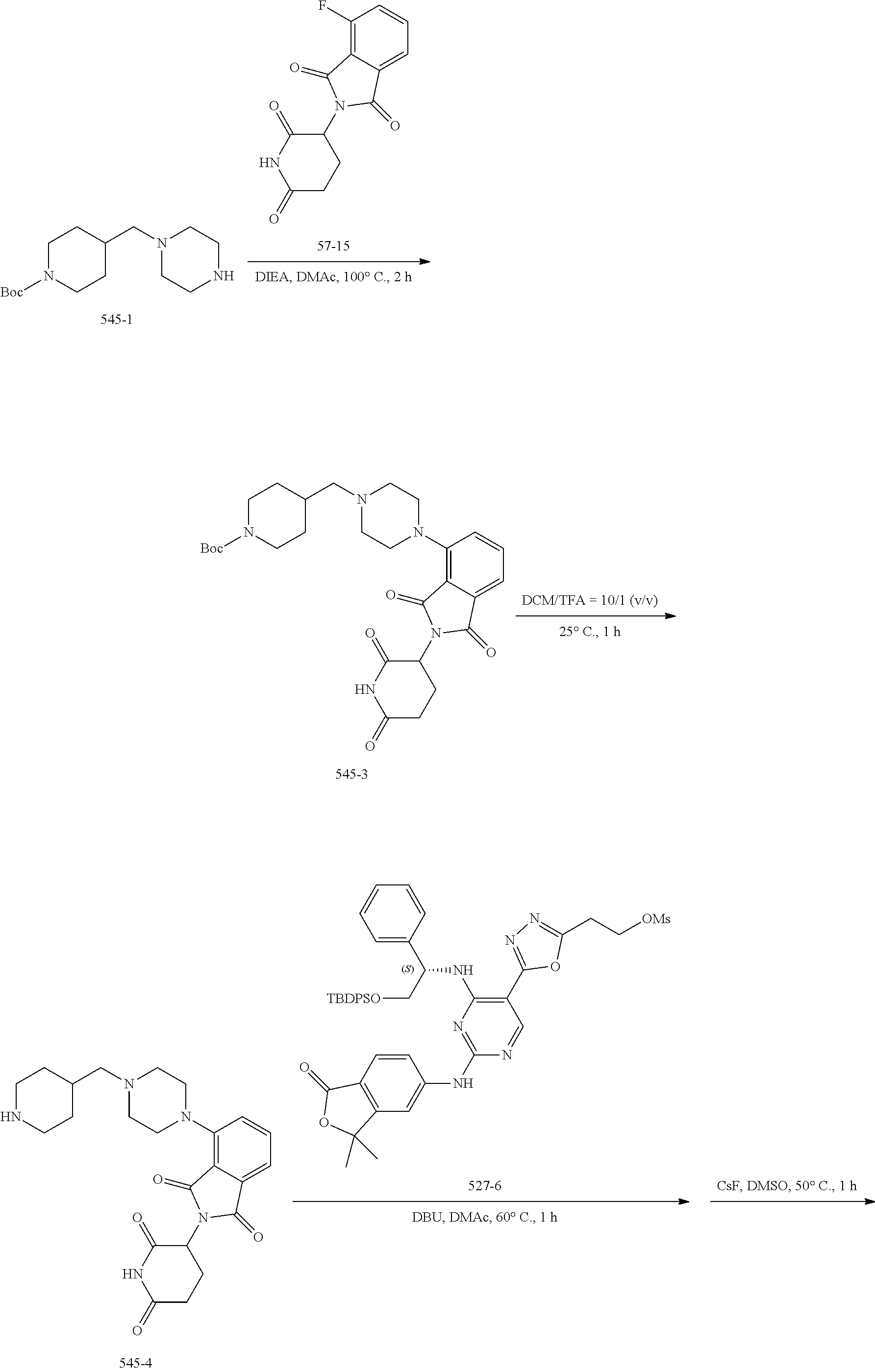

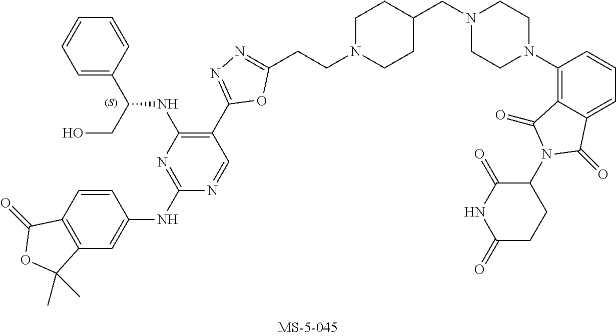

The procedure for preparing compound 527-6 is described in the synthesis of MS-5-029.

Procedure for preparation of compound 545-3. To a solution of compound 545-1 (1.5 g, 5.29 mmol, 1 eq) in DMAc (15 mL) was added DIEA (1.37 g, 10.59 mmol, 1.84 mL, 2 eq) and compound 57-15 (1.46 g, 5.29 mmol, 1 eq). The mixture was stirred at 100° C. for 2 h. The reaction mixture was filtered and concentrated under reduced pressure to give compound 545-3 (1.24 g, crude) as a yellow solid. LCMS: t_{R}=0.335 min, MS (ESI+) m/z=540.4[M+1]⁺. ¹H NMR (400 MHz, DMSO-d₆) δ=11.08 (s, 1H), 7.69 (dd, J=8.4, 7.2 Hz, 1H), 7.17-7.44 (m, 2H), 5.08 (dd, J=12.8, 5.4 Hz, 1H), 3.91 (d, J=11.6 Hz, 2H), 3.28 (s, 4H), 2.84-2.92 (m, 1H), 2.65-2.76 (m, 2H), 2.60 (d, J=2.8 Hz, 2H), 2.50-2.55 (m, 4H), 2.18 (d, J=6.8 Hz, 2H), 1.97-2.07 (m, 1H), 1.67 (s, 3H), 1.38 (s, 9H), 0.86-1.05 (m, 2H).

Procedure for preparation of compound 545-4. To a solution of compound 545-3 (1.24 g, 2.29 mmol, 1 eq) in DCM (10 mL) was added TFA (1 mL), and then the mixture was stirred at 25° C. for 1 h. The mixture was concentrated in vacuum to give compound 545-4 (1 g, crude, TFA) as a yellow oil. LCMS: t_{R}=0.157 min, MS (ESI+) m/z=440.2[M+1]⁺ Procedure for preparation of MS-5-045. To a solution of compound 545-4 (500 mg, 1.14 mmol, 1 eq) in DBU (865.95 mg, 5.69 mmol, 857.38 μL, 5 eq) was added DMAc (5 mL) and compound 527-6 (931.74 mg, 1.14 mmol, 1 eq). The mixture was stirred at 60° C. for 1 h. The mixture was concentrated in vacuum to give a residue. The residue was purified by prep-HPLC (40%-70% ACN in water (0.225% FA), 10 min) and lyophilized to give MS-5-045 (54 mg, 58.38 μmol, 5.13% yield, 99.9% purity) as a yellow solid. LCMS: t_{R}=0.326 min, MS (ESI+) m/z=462.9[M+1]⁺. ¹H NMR (400 MHz, DMSO-d₆) δ=11.08 (s, 1H), 10.29 (s, 1H), 8.88 (d, J=7.6 Hz, 1H), 8.66 (s, 1H), 8.20 (s, 1H), 7.98 (s, 1H), 7.58-7.76 (m, 3H), 7.21-7.48 (m, 7H), 5.40-5.48 (m, 1H), 5.19-5.29 (m, 1H), 5.09 (dd, J=12.8, 5.6 Hz, 1H), 3.89-3.95 (m, 1H), 3.82-3.88 (m, 1H), 3.12 (t, J=7.2 Hz, 4H), 2.75-2.98 (m, 7H), 2.54-2.64 (m, 6H), 2.18 (d, J=7.2 Hz, 2H), 1.96-2.06 (m, 3H), 1.71 (d, J=12.0 Hz, 2H), 1.56 (d, J=19.6 Hz, 7H), 1.03-1.15 (m, 2H).

### Example 33

### Synthesis of MS-5-046

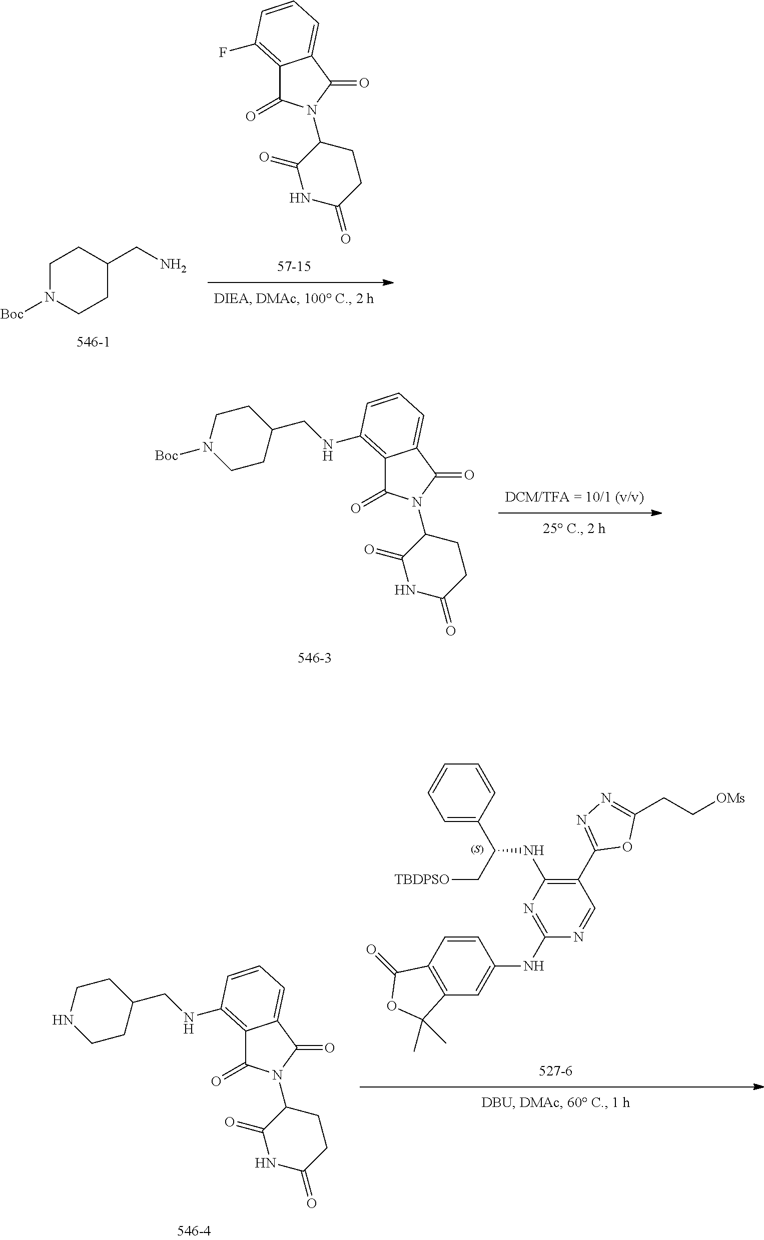

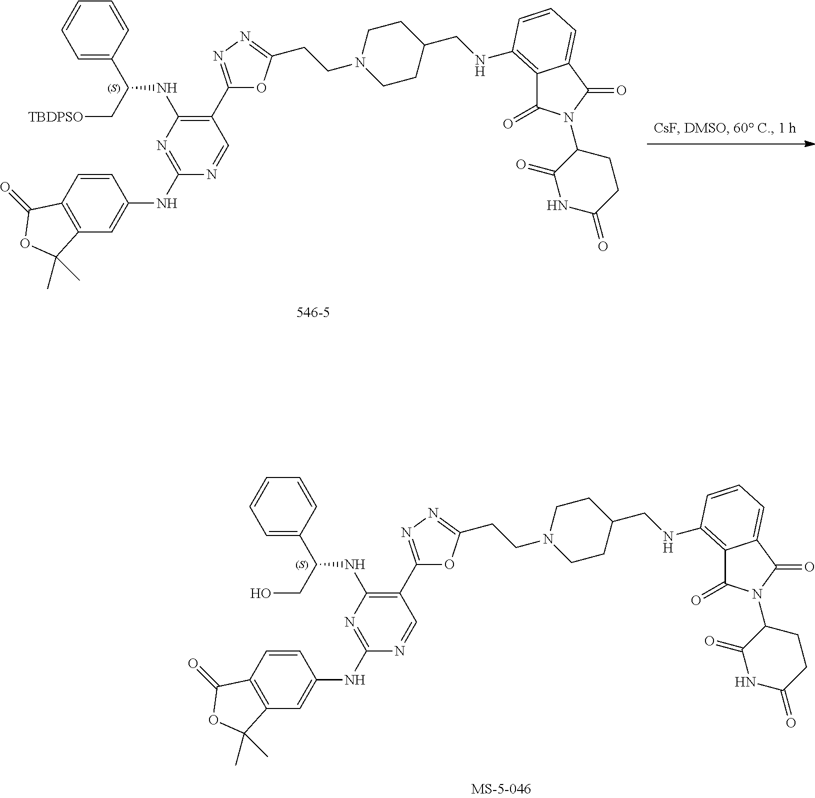

The procedure for preparing compound 527-6 is described in the synthesis of MS-5-029.

Procedure for preparation of compound 546-3. To a solution of compound 546-1 (2 g, 9.33 mmol, 1 eq) and compound 57-15 (2.58 g, 9.33 mmol, 1 eq) in DMAc (15 mL) was added DIEA (3.62 g, 28.00 mmol, 4.88 mL, 3 eq). The mixture was stirred at 100° C. for 2 h. The residue was purified by prep-HPLC (45%-75% ACN in water (0.225% FA), 30 min) and lyophilized to give compound 546-3 (1.8 g, 3.83 mmol, 40.99% yield) as a green solid. LCMS: t_{R}=0.488 min, MS (ESI+) m/z=493.1[M+1]⁺. ¹H NMR (400 MHz, DMSO-d₆) δ=11.09 (s, 1H), 7.68-7.47 (m, 1H), 7.15 (d, J 8.8 Hz, 1H), 7.02 (d, J 6.8 Hz, 1H), 6.63 (t, J 6.0 Hz, 1H), 5.04 (dd, J 13.2, 5.6 Hz, 1H), 3.94 (d, J 10.8 Hz, 2H), 3.22 (t, J 6.4 Hz, 2H), 2.97-2.80 (m, 1H), 2.64-2.52 (m, 3H), 2.07-1.97 (m, 1H), 1.83-1.72 (m, 1H), 1.67 (d, J 12.8 Hz, 2H), 1.39 (s, 9H), 1.07 (m, 2H).

Procedure for preparation of compound 546-4. To a solution of compound 546-3 in DCM (3 mL) was added TFA (0.3 mL). The mixture was stirred at 25° C. for 2 h. The mixture was filtered and concentrated in vacuum to give compound 546-4 (100 mg, crude, TFA) as a green oil. LCMS: t_{R}=0.261 min, MS (ESI+) m/z=371.2[M+1]⁺

Procedure for preparation of compound 546-5. To a solution of compound 546-4 (95 mg, 256.48 mol, 1 eq) in DMAc (1 mL) was added DBU (195.23 mg, 1.28 mmol, 193.29 μL, 5 eq) and compound 527-6 (210.06 mg, 256.48 μmol, 1 eq). The mixture was stirred at 60° C. for 1 h. The residue was purified by prep-HPLC (36%-66% ACN in water (0.225% FA), 10 min) and lyophilized to give compound 546-5 (80 mg, 70.98 μmol, 27.67% yield, 97% purity, FA) as a yellow solid. LCMS: t_{R}=0.518 min, MS (ESI+) m/z=1093.7[M+1]⁺. ¹H NMR (400 MHz, DMSO-d₆) δ=11.04-11.13 (m, 1H), 10.29 (s, 1H), 9.22 (d, J=8.4 Hz, 1H), 8.68 (s, 1H), 8.21-8.27 (m, 1H), 8.23 (s, 1H), 7.88 (s, 1H), 7.70-7.75 (m, 1H), 7.55-7.63 (m, 2H), 7.51-7.54 (m, 2H), 7.37-7.42 (m, 6H), 7.29-7.35 (m, 4H), 7.27 (s, 3H), 7.11 (d, J=8.8 Hz, 1H), 7.01 (d, J=6.8 Hz, 1H), 6.58 (t, J=6.4 Hz, 1H), 5.53-5.60 (m, 1H), 5.03 (dd, J=12.8, 5.6 Hz, 1H), 4.13 (dd, J=10.2, 3.6 Hz, 1H), 3.97 (dd, J=10.0, 3.2 Hz, 1H), 3.15 (d, J=14.8 Hz, 4H), 2.96 (d, J=10.4 Hz, 2H), 2.85-2.89 (m, 1H), 2.79-2.83 (m, 2H), 2.54-2.60 (m, 2H), 2.00 (d, J=10.8 Hz, 3H), 1.68 (d, J=11.6 Hz, 2H), 1.51 (d, J=4.8 Hz, 6H), 1.17-1.24 (m, 2H), 0.90 (s, 9H).

Procedure for preparation of compound MS-5-046. To a solution of compound 546-5 (70 mg, 64.03 μmol, 1 eq) in DMSO (1 mL) was added CsF (9.73 mg, 64.03 μmol, 2.36 μL, 1 eq). The mixture was stirred at 60° C. for 1 h. The residue was purified by prep-HPLC (14%-44% ACN in water (0.225% FA), 10 min) and lyophilized to give MS-5-046 (41 mg, 47.48 μmol, 74.16% yield, 99% purity) as a yellow solid. LCMS: t_{R}=0.383 min, MS (ESI+) m/z=855.5[M+1]⁺. ¹H NMR (400 MHz, DMSO-d₆) δ=11.09 (s, 1H), 10.29 (s, 1H), 8.87 (d, J=8.0 Hz, 1H), 8.65 (s, 1H), 8.17 (s, 1H), 7.97 (s, 1H), 7.68-7.73 (m, 1H), 7.61-7.65 (m, 1H), 7.56 (dd, J=8.8, 7.2 Hz, 1H), 7.34-7.42 (m, 4H), 7.22-7.30 (m, 1H), 7.13 (d, J=8.8 Hz, 1H), 7.01 (d, J=7.2 Hz, 1H), 6.59 (t, J=6.0 Hz, 1H), 5.43 (d, J=7.6 Hz, 1H), 5.23 (s, 1H), 5.04 (dd, J=12.8, 5.6 Hz, 1H), 3.81-3.91 (m, 2H), 3.20 (s, 2H), 3.08-3.12 (m, 2H), 2.93-2.97 (m, 2H), 2.83-2.89 (m, 1H), 2.78 (t, J=7.2 Hz, 2H), 2.55-2.63 (m, 2H), 1.92-2.05 (m, 3H), 1.64-1.72 (m, 2H), 1.57 (s, 3H), 1.52 (s, 3H), 1.14-1.24 (m, 2H).

### Example 34

### Synthesis of MS-6-003

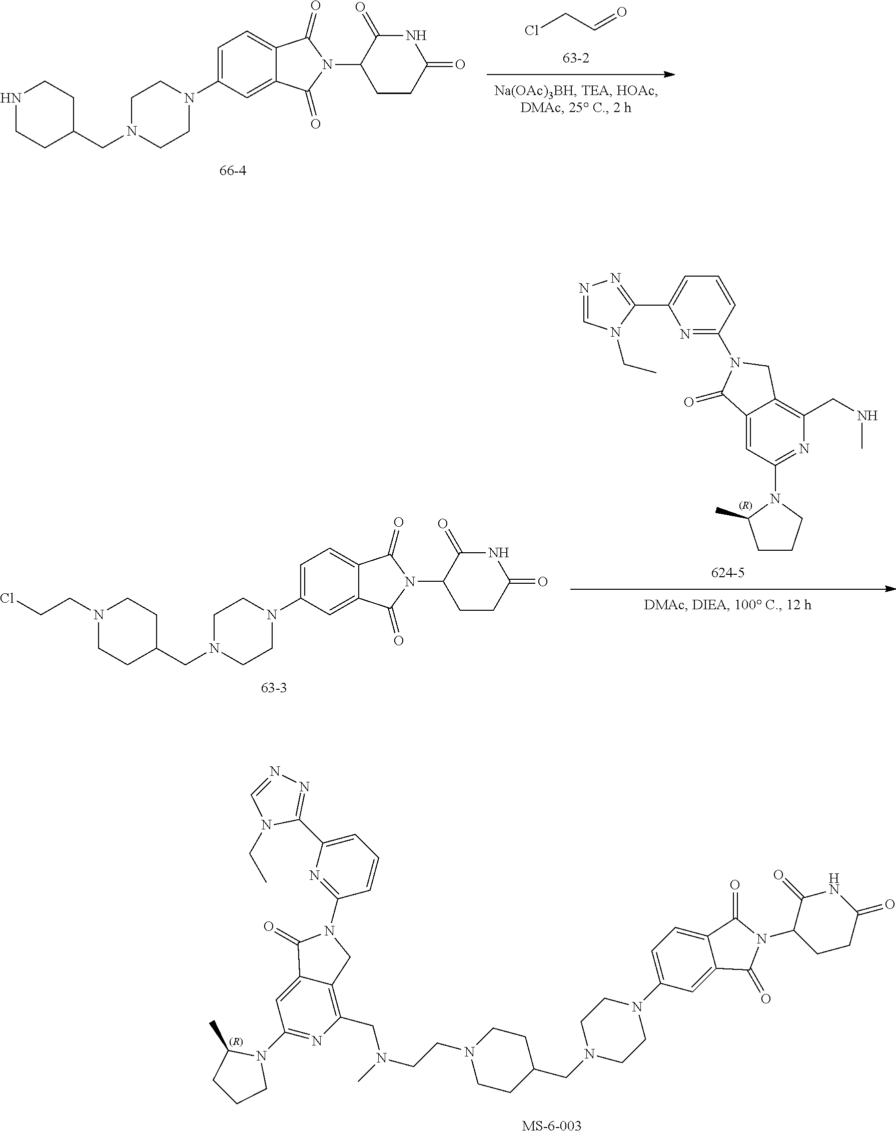

The procedure for preparing compound 66-4 is described in the synthesis of MS-6-006.

The procedure for preparing compound 624-5 is described in synthesis of MS-6-027.

Procedure for preparation of compound 63-3. To a solution of compound 66-4 (500 mg, 1.05 mmol, 1 eq, HCl) and compound 63-2 (206.06 mg, 1.05 mmol, 168.90 uL, 40% purity, 1 eq) in DMAc (5 mL) and HOAc (0.5 mL) was added borane;2-methylpyridine (112.36 mg, 1.05 mmol, 1 eq) and the mixture was stirred at 25° C. for 2 h. The reaction mixture was filtered and purified by prep-HPLC (0%-15% ACN in water (0.225% FA), 10 min) to give compound 63-3 (150 mg, 273.71 μmol, 26.07% yield, FA) as a yellow solid. LCMS: t_{R}=0.226 min, MS (ESI+) m/z=502.0[M+1]⁺. ¹H NMR (EC6208-153-P1A) (400 MHz, DMSO-d₆): δ=11.09 (s, 1H), 8.15 (s, 1H), 7.68 (dd, J=1.6, 8.4 Hz, 1H), 7.40-7.21 (m, 2H), 5.07 (dd, J=5.2, 12.8 Hz, 1H), 4.40-3.51 (m, 9H), 3.27-2.52 (m, 9H), 2.45-1.06 (m, 9H).

Procedure for preparation of MS-6-003. A mixture of compound 63-3 (20 mg, 39.84 μmol, 1 eq), compound 624-5 (17.23 mg, 39.84 μmol, 1 eq), DIEA (25.75 mg, 199.20 μmol, 34.70 uL, 5 eq) and KI (13.23 mg, 79.68 μmol, 2 eq) in DMAc (0.5 mL) was stirred at 100° C. for 12 h. The reaction mixture was filtered and purified by prep-HPLC (47%-77% ACN in water (10 mM NH₄HCO₃), 9 min) and prep-HPLC (B %: 2%-32% ACN in water (0.05% HCl), 8 min) to give MS-6-003 (6.20 mg, 6.63 μmol, 16.65% yield, HCl) as a yellow solid. LCMS (EC6208-161-P1G): t_{R}=0.333 min, MS (ESI+) m/z=898.5[M+1]⁺. ¹H NMR (EC6208-161-P1A) (400 MHz, DMSO-d₆): δ=11.30 (s, 2H), 11.10 (s, 1H), 9.25 (s, 1H), 8.67 (d, J=8.4 Hz, 1H), 8.16 (t, J=8.0 Hz, 1H), 8.02 (d, J=7.6 Hz, 1H), 7.76 (d, J=8.4 Hz, 1H), 7.50 (s, 1H), 7.36 (d, J=8.4 Hz, 1H), 6.82 (s, 1H), 5.29 (s, 2H), 5.10 (dd, J=5.6, 12.8 Hz, 1H), 4.72 (q, J=7.2 Hz, 2H), 4.59 (br s, 2H), 4.34 (d, J=4.4 Hz, 1H), 4.20 (d, J=11.8 Hz, 2H), 3.91 (s, 3H), 3.64-3.58 (m, 6H), 3.51-3.36 (m, 2H), 3.18-2.95 (m, 8H), 2.94-2.84 (m, 1H), 2.71-2.53 (m, 3H), 2.28-1.93 (m, 7H), 1.82-1.58 (m, 3H), 1.52 (t, J=7.2 Hz, 3H), 1.32-1.18 (m, 4H).

### Example 35

### Synthesis of MS-6-004

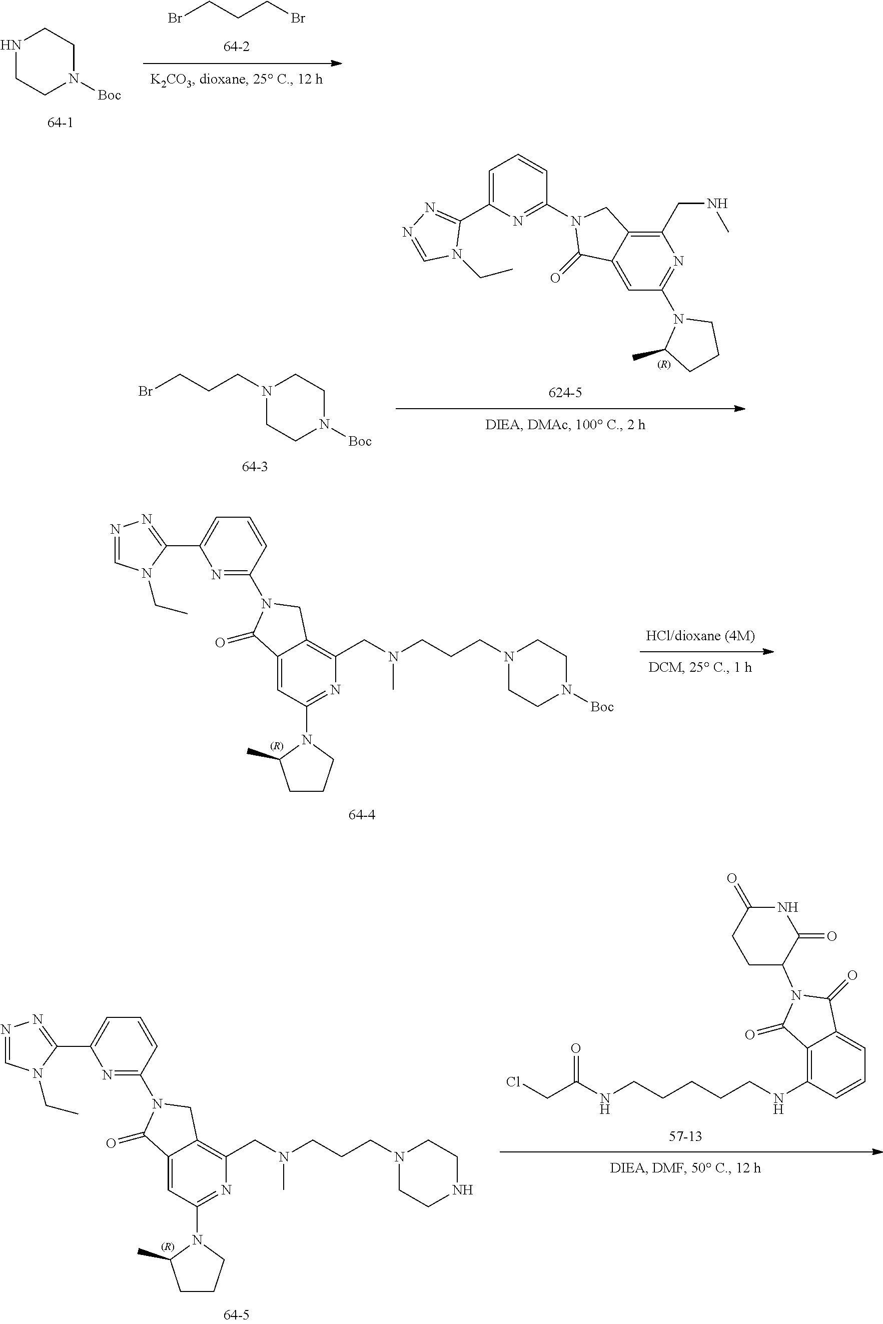

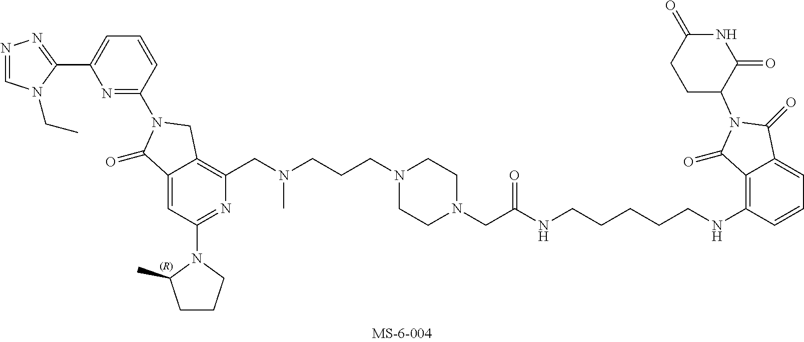

The procedure for preparing 624-5 is described in the synthesis of MS-6-027.

For the procedure for preparing compound 57-13 please see synthesis of MS-5-007.

Procedure for preparation of compound 64-3. To a solution of compound 64-1 (5 g, 26.85 mmol, 1 eq) and compound 64-2 (54.20 g, 268.46 mmol, 27.37 mL, 10 eq) in dioxane (100 mL) was added K₂CO₃ (18.55 g, 134.23 mmol, 5 eq). The mixture was stirred at 25° C. for 12 h. The reaction mixture was filtered and the filtrate was concentrated under the reduced pressure to give a residue. The residue was purified by flash silica gel chromatography (SiO₂, DCM/MeOH=100/1 to 10/1) to give compound 64-3 (5.62 g, 18.28 mmol, 68.10% yie) as a colourless solid. LCMS: t_{R}=0.268 min, MS (ESI+) m/z=307.1[M+1]⁺. ¹H NMR (400 MHz, CDCl₃): δ=3.69-3.63 (m, 1H), 3.43 (t, J=6.4 Hz, 2H), 3.40-3.27 (m, 4H), 2.44 (t, J=6.8 Hz, 2H), 2.39-2.26 (m, 4H), 1.99-1.93 (m, 1H), 1.42 (s, 9H).

Procedure for preparation of compound 64-4. To a solution of intermediate 3 (587.12 mg, 1.25 mmol, 1 eq, HCl) in DMAc (3 mL) was added DIEA (485.40 mg, 3.76 mmol, 654.17 uL, 3 eq) and compound 64-3 (500 mg, 1.63 mmol, 1.3 eq). The mixture was stirred at 100° C. for 2 h. The reaction mixture purified by prep-HPLC (10%-40% ACN in water (0.225% FA), 10 min) and lyophilized to give compound 64-4 (127 mg, 192.76 μmol, 15.40% yield) as a yellow oil. LCMS: t_{R}=0.367 min, MS (ESI+) m/z=659.3 [M+1]⁺. ¹H NMR (400 MHz, CDCl₃): δ=8.85-8.65 (m, 1H), 8.28 (s, 1H), 8.16 (d, J=6.8 Hz, 1H), 7.94 (t, J=8.0 Hz, 1H), 6.72 (d, J=2.8 Hz, 1H), 5.24-5.00 (m, 2H), 4.71 (q, J=6.8 Hz, 2H), 4.30-4.18 (m, 1H), 3.87-3.68 (m, 2H), 3.66-3.57 (m, 1H), 3.57-3.27 (m, 6H), 2.59-2.23 (m, 10H), 2.12 (d, J=3.2 Hz, 2H), 1.79-1.66 (m, 4H), 1.60 (t, J=7.2 Hz, 3H), 1.46 (s, 9H), 1.26 (d, J=6.8 Hz, 3H)

Procedure for preparation of compound 64-5. To a solution of compound 64-4 (127 mg, 192.76 μmol, 1 eq) in DCM (1.5 mL) was added HCl/dioxane (4 M, 240.96 uL, 5 eq). The mixture was stirred at 25° C. for 1 h. The reaction mixture was filtered and the filter residue was concentrated under the reduced pressure to give compound 64-5 (110 mg, crude, HCl) as a yellow solid. LCMS: t_{R}=0.310 min, MS (ESI+) m/z=559.4 [M+1]⁺ Procedure for preparation of MS-6-004. To a solution of compound 64-5 (55 mg, 92.41 μmol, 1 eq, HCl) and compound 57-13 (40.19 mg, 92.41 μmol, 1 eq) in DMF (1 mL) was added DIEA (35.83 mg, 277.23 μmol, 48.29 uL, 3 eq). The mixture was stirred at 50° C. for 12 h. The reaction mixture was purified by prep-HPLC (9%-39% ACN in water (0.225% FA), 9 min) and lyophilized to give MS-6-004 (24.51 mg, 24.43 μmol, 26.44% yield, 100% purity, FA) as a yellow solid. LCMS: t_{R}=0.387 min, MS (ESI+) m/z=957.8 [M+1]⁺. ¹H NMR (400 MHz, DMSO-d₆): δ=11.09 (s, 1H), 8.75 (s, 1H), 8.62 (d, J=8.4 Hz, 1H), 8.15 (s, 1H), 8.12-8.03 (m, 1H), 7.98 (d, J=7.6 Hz, 1H), 7.68-7.44 (m, 2H), 7.06 (d, J=8.8 Hz, 1H), 6.99 (d, J=6.8 Hz, 1H), 6.59 (s, 1H), 6.50 (t, J=5.6 Hz, 1H), 5.13 (s, 2H), 5.04 (dd, J=4.8, 12.8 Hz, 1H), 4.63 (q, J=7.2 Hz, 2H), 4.24-4.14 (m, 1H), 3.66 (s, 2H), 3.55-3.51 (m, 2H), 3.25 (d, J=6.4 Hz, 2H), 3.10-3.05 (m, 2H), 2.96-2.82 (m, 2H), 2.79 (s, 2H), 2.61-2.55 (m, 2H), 2.42-2.16 (m, 14H), 2.13-1.95 (m, 4H), 1.69 (s, 1H), 1.63-1.38 (m, 9H), 1.37-1.25 (m, 2H), 1.19 (d, J=6.0 Hz, 3H).

### Example 36

### Synthesis of MS-6-005

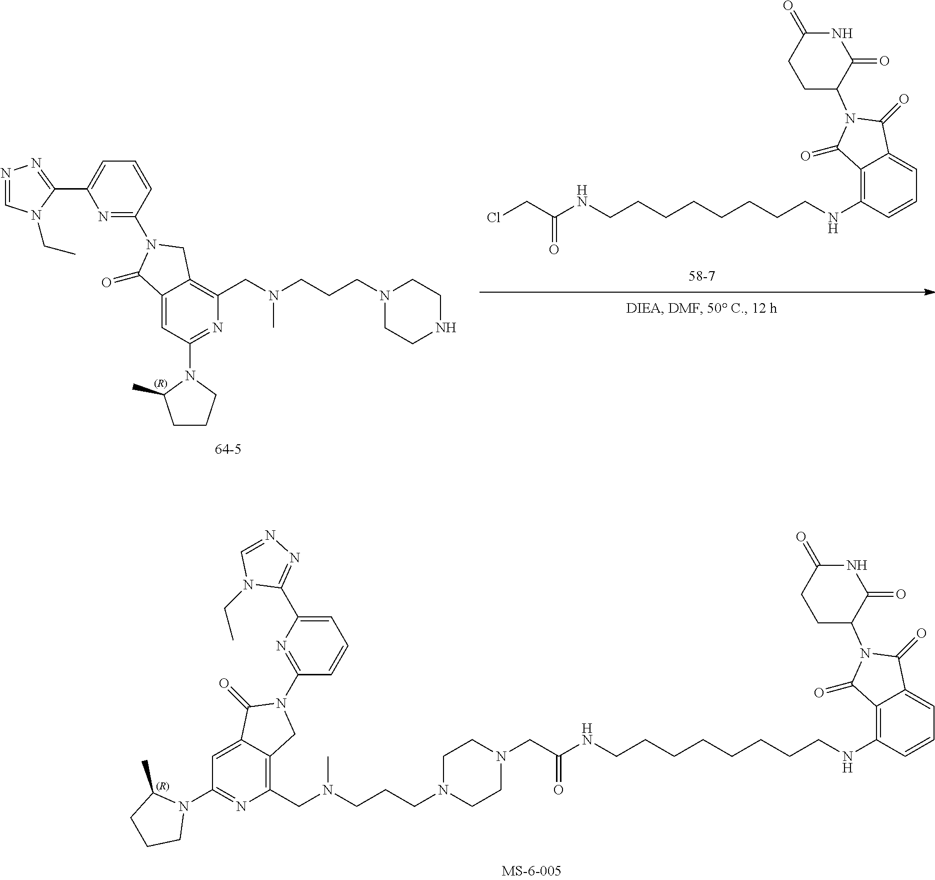

For the procedure for preparing compound 64-5 please see synthesis of MS-6-004.

For the procedure for preparing compound 58-7 please see synthesis of MS-5-008.

Procedure for preparation of MS-6-005. To a solution of compound 64-5 (55 mg, 92.41 μmol, 1 eq, HCl) and compound 58-7 (44.07 mg, 92.41 μmol, 1 eq) in DMF (1 mL) was added DIEA (35.83 mg, 277.23 μmol, 48.29 uL, 3 eq). The mixture was stirred at 50° C. for 12 h. The reaction mixture was purified by prep-HPLC (16%-46% ACN in water (0.225% FA), 9 min) to give MS-6-005 (25.12 mg, 23.92 μmol, 25.89% yield, 99.54% purity, FA) as a yellow solid. LCMS: t_{R}=0.420 min, MS (ESI+) m/z=1000.5 [M+1]⁺. ¹H NMR (400 MHz, DMSO-d₆): δ=11.09 (s, 1H), 8.75 (s, 1H), 8.61 (d, J=8.4 Hz, 1H), 8.14 (s, 1H), 8.11-8.03 (m, 1H), 8.02-7.90 (m, 1H), 7.68-7.45 (m, 2H), 7.04 (d, J=8.4 Hz, 1H), 6.99 (d, J=7.2 Hz, 1H), 6.58 (s, 1H), 6.48 (t, J=5.6 Hz, 1H), 5.12 (s, 2H), 5.04 (m, 1H), 4.63 (m, 2H), 4.25-4.14 (m, 1H), 3.66 (s, 2H), 3.52 (m, 2H), 3.26 (d, J=7.2 Hz, 2H), 3.09-3.03 (m, 2H), 2.94-2.81 (m, 2H), 2.79 (s, 2H), 2.60 (d, J=1.8 Hz, 2H), 2.43-2.16 (m, 14H), 2.11-1.93 (m, 4H), 1.69 (, 1H), 1.63-1.45 (m, 7H), 1.43-1.12 (m, 13H)

### Example 37

### Synthesis of MS-6-006

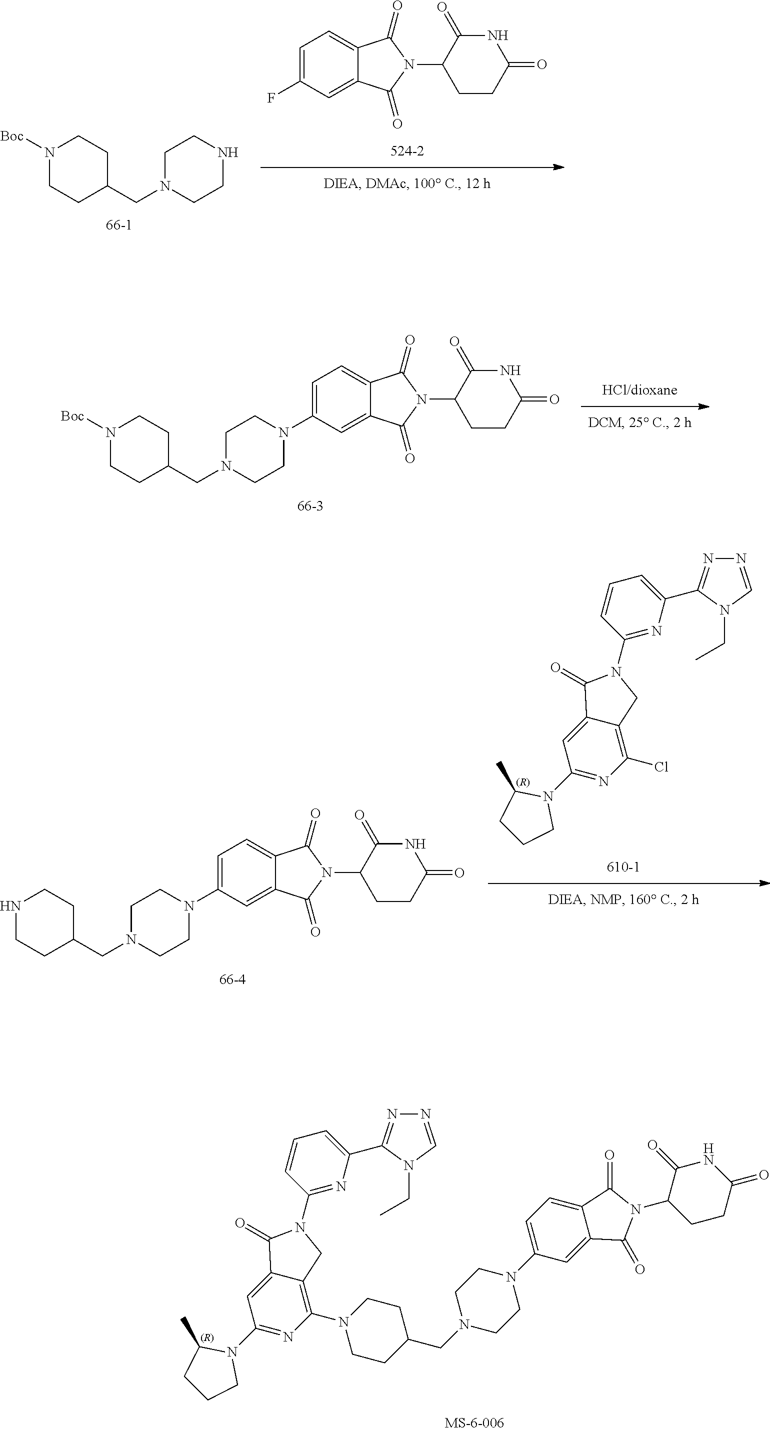

Procedure for preparation of compound 66-3. To a solution of compound 66-1 (4 g, 14.11 mmol, 1 eq) and compound 524-2 (3.90 g, 14.11 mmol, 1 eq) in DMAc (25 mL) was added DIEA (5.47 g, 42.34 mmol, 7.38 mL, 3 eq) and the mixture was stirred at 90° C. for 4 h. The reaction mixture was quenched with H₂O (80 mL) and filtered, the cake was dried in vacuum to give a residue. The crude product was triturated with MeCN (50 mL) at 25° C. for 30 min to give compound 66-3 (5.6 g, 10.38 mmol, 73.53% yield) as a yellow solid. LCMS: t_{R}=0.331 min, MS (ESI+) m/z=540.1[M+1]⁺. ¹H NMR (400 MHz, CDCl₃): δ=8.22 (s, 1H), 7.70 (d, J=8.4 Hz, 1H), 7.30 (d, J=2.0 Hz, 1H), 7.07 (dd, J=2.0, 8.4 Hz, 1H), 4.96 (dd, J=5.2, 12.4 Hz, 1H), 4.11 (s, 2H), 3.52-3.37 (m, 4H), 3.06-2.64 (m, 5H), 2.62-2.54 (m, 4H), 2.25 (d, J=7.2 Hz, 2H), 2.20-2.08 (m, 1H), 1.85-1.61 (m, 3H), 1.48 (s, 9H), 1.20-1.03 (m, 2H).

Procedure for preparation of compound 66-4. To a solution of compound 66-3 (2 g, 3.71 mmol, 1 eq) in DCM (10 mL) was added HCl/dioxane (4 M, 10 mL, 10.79 eq) and the mixture was stirred at 25° C. for 2 h. The reaction mixture was concentrated under reduced pressure to give compound 66-4 (6 g, crude, HCl) as a yellow solid, used directly. LCMS: t_{R}=0.311 min, MS (ESI+) m/z=439.9[M+1]⁺. ¹H NMR (400 MHz, DMSO-d₆): δ=11.38 (s, 1H), 11.09 (s, 1H), 9.41-8.96 (m, 2H), 7.75 (d, J=8.4 Hz, 1H), 7.49 (d, J=1.6 Hz, 1H), 7.36 (dd, J=2.0, 8.4 Hz, 1H), 5.09 (dd, J=5.6, 12.8 Hz, 1H), 4.81 (s, 4H), 4.18 (d, J=13.6 Hz, 2H), 3.70-3.62 (m, 2H), 3.25 (d, J=12.4 Hz, 2H), 3.17-3.00 (m, 4H), 2.91-2.84 (m, 2H), 2.65-2.52 (m, 2H), 2.20 (s, 1H), 2.08-2.03 (m, 2H), 1.56-1.39 (m, 2H).

See below for the procedure for preparation of compound 610-1:

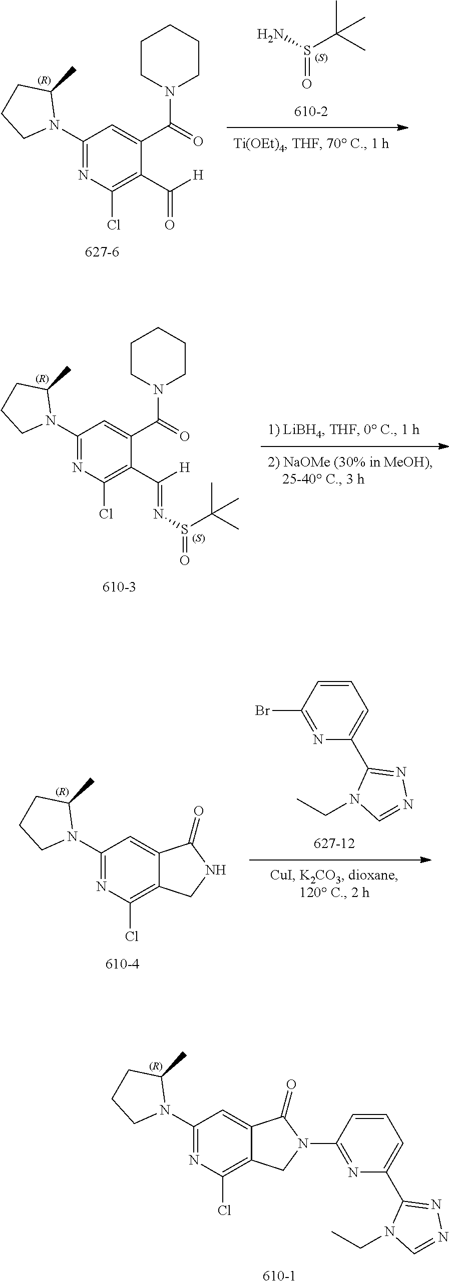

The procedure for preparing compound 627-6 & 627-12 are described in the synthesis of MS-6-027.

Procedure for preparation of compound 610-3. To a solution of compound 627-6 (33 g, 98.26 mmol, 1 eq), compound 610-2 (23.82 g, 196.53 mmol, 2 eq) and Ti(OEt)₄ (56.04 g, 245.66 mmol, 50.94 mL, 2.5 eq) in THE (270 mL) was degassed and purged with N₂ for 3 times, and then the mixture was stirred at 70° C. for 1 h under N₂ atmosphere. The reaction mixture was poured into water (500 mL) and extracted with ethyl acetate (300 mL×3). The combined organic phase was dried by over Na₂SO₄, filtered and concentrated in vacuo to afford crude. The residue was purified by flash silica gel chromatography (SiO₂, Petroleum ether/Ethyl acetate=100/1 to 2/1) to give compound 610-3 (26.7 g, 60.82 mmol, 61.89% yield) as a yellow solid. LCMS: t_{R}=0.519 min, MS (ESI+) m/z=439.3 [M+1]⁺. ¹H NMR (400 MHz, CDCl₃): δ=8.82 (s, 1H), 6.13 (s, 1H), 3.95 (d, J=12.0 Hz, 1H), 3.32-3.10 (m, 3H), 2.99 (m, 1H), 2.09-1.97 (m, 3H), 1.81-1.63 (m, 4H), 1.57-1.48 (m, 3H), 1.43-1.27 (m, 2H), 1.20-1.15 (m, 12H)

Procedure for preparation of compound 610-4. To give compound 610-3 (26 g, 59.22 mmol, 1 eq) in THE (260 mL) was added dropwise LiBH₄ (1.42 g, 65.19 mmol, 1.10 eq) at 0° C. After addition, the mixture was stirred for 1 h, and then NaOMe (53.32 g, 296.12 mmol, 30% purity, eq) was added dropwise at 25° C. The resulting mixture was stirred at 40° C. for 3 h. The mixture was quenched with NH₄Cl aqueous solution (500 mL). The mixture was extracted with EtOAc (200 mL×3). The combined organic phase was washed with brine (500 mL), dried with anhydrous Na₂SO₄, filtered and concentrated in vacuum. The residue was purified by flash silica gel chromatography (SiO₂, Petroleum ether/Ethyl acetate=100/1 to 2/1) to give compound 610-4 (8.79 g, 34.92 mmol, 58.96% yield) as a white solid. LCMS: t_{R}=0.432 min, MS (ESI+) m/z=251.9 [M+1]⁺. ¹H NMR (400 MHz, CDCl₃): δ=7.37 (d, J=1.5 Hz, 1H), 6.66 (s, 1H), 4.35 (s, 2H), 4.17 (t, J=5.8 Hz, 1H), 3.67-3.49 (m, 1H), 3.45-3.31 (m, 1H), 2.19-1.92 (m, 3H), 1.83-1.59 (m, 1H), 1.23 (d, J=6.4 Hz, 3H).

Procedure for preparation of 610-1. To a solution of compound 610-4 (8.5 g, 33.77 mmol, 1 eq), compound 627-12 (10.26 g, 40.52 mmol, 1.2 eq), CuI (1.61 g, 8.44 mmol, 0.25 eq) and K₂CO₃ (10.27 g, 74.29 mmol, 2.2 eq) in dioxane (170 mL) was degassed and purged with N₂ for 3 times, and then the mixture was stirred at 120° C. for 2 h under N₂ atmosphere. The mixture was filtered and concentrated in vacuum to give a residue. The residue was triturated with water at 20° C. for 10 min to give 610-1 (13.6 g, 24.29 mmol, 71.92% yield, 75.7% purity) as a brown solid. 100 mg of the crude product was purified by prep-HPLC (45%-70% ACN in water (0.225% FA), 22 min) and lyophilized to give MS-6-010 (30.08 mg, 70.64 μmol, 99.55% purity) as a yellow solid. LCMS: t_{R}=0.510 min, MS (ESI+) m/z=424.0 [M+1]⁺. ¹H NMR (400 MHz, CDCl₃): δ=8.70 (dd, J=8.4, 0.4 Hz, 1H), 8.29 (s, 1H), 8.21 (dd, J=7.6, 0.8 Hz, 1H), 7.99-7.91 (m, 1H), 6.73 (s, 1H), 4.98 (s, 2H), 4.70 (m, 2H), 4.22 (br t, J=6.4 Hz, 1H), 3.62 (m, 1H), 3.43 (m, 1H), 2.28-1.98 (m, 3H), 1.78 (m, 1H), 1.63 (t, J=7.2 Hz, 3H), 1.27 (d, J=6.4 Hz, 3H).

Procedure for preparation of MS-6-006. 610-1 (100 mg, 235.91 μmol, 1 eq), compound 66-4 (112.28 mg, 235.91 μmol, 1 eq, HCl) and DIEA (91.47 mg, 707.72 μmol, 123.27 uL, 3 eq) were taken up into a microwave tube in NMP (1.5 mL). The sealed tube was microwaved at 145° C. for 2 h. The reaction mixture was filtered and purified by prep-HPLC (16%-46% ACN in water (0.225% FA), 15 min) to give MS-6-006 (16.25 mg, 18.67 μmol, 7.91% yield, 95% purity) as a yellow solid. LCMS: t_{R}=0.435 min, MS (ESI+) m/z=827.2[M+1]⁺. ¹H NMR (400 MHz, DMSO-d₆): δ=11.09 (s, 1H), 8.74 (s, 1H), 8.63 (d, J=8.4 Hz, 1H), 8.12-8.04 (m, 1H), 8.03-7.95 (m, 1H), 7.69 (d, J=8.4 Hz, 1H), 7.36 (s, 1H), 7.32-7.23 (m, 1H), 6.06 (s, 1H), 5.19 (s, 2H), 5.12-5.02 (m, 1H), 4.62 (q, J=6.8 Hz, 2H), 4.31-4.14 (m, 3H), 3.55-3.41 (m, 5H), 3.07-2.82 (m, 4H), 2.61 (, 4H), 2.27-2.17 (m, 2H), 2.07-1.78 (m, 8H), 1.67 (s, 1H), 1.50 (t, J=7.2 Hz, 3H), 1.26-1.15 (m, 5H).

### Example 38

### Synthesis for MS-6-007

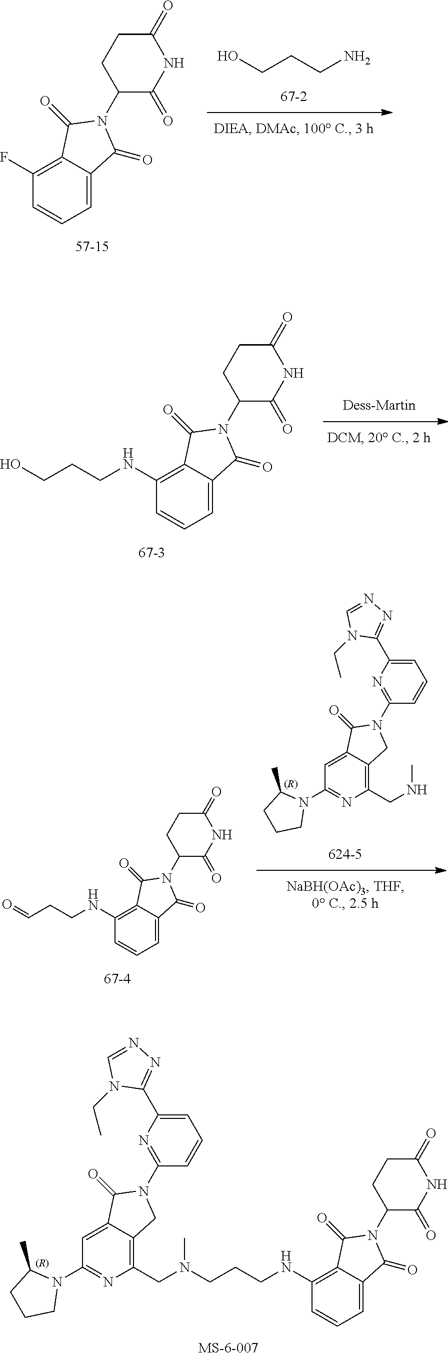

The procedure for preparing compound 624-5 is described in the synthesis of MS-6-027.

Procedure for preparation of compound 67-3. To a solution of compound 67-2 (326.30 mg, 4.34 mmol, 335.01 uL, 1.2 eq) in DMAc (10 mL) was added compound 57-15 (commercially available, 1 g, 3.62 mmol, 1 eq) and DIEA (1.40 g, 10.86 mmol, 1.89 mL, 3 eq) and the mixture was stirred at 100° C. for 3 h. The reaction mixture was quenched with H₂O (20 mL) and extracted with EtOAc (30 mL×3), the combined organic layers were concentrated under reduced pressure to give a residue. The residue was purified by prep-HPLC (9%-39% ACN in water (0.05% HCl), 10 min) to give compound 67-3 (264 mg, 796.81 μmol, 22.01% yield) as a yellow powder. LCMS: t_{R}=0.323 min, MS (ESI+) m/z=331.8[M+1]⁺. ¹H NMR (400 MHz, DMSO-d₆): δ=11.09 (s, 1H), 7.59 (dd, J=8.4, 7.2 Hz, 1H), 7.28-6.92 (m, 2H), 6.88-6.56 (m, 1H), 5.05 (dd, J=5.6, 12.8 Hz, 1H), 3.52 (t, J=6.0 Hz, 3H), 3.37 (d, J=5.6 Hz, 2H), 2.89 (dd, J=14.0, 5.6 Hz, 1H), 2.65-2.53 (m, 2H), 2.14-1.96 (m, 1H), 1.84-1.68 (m, 2H).

Procedure for preparation of compound 67-4. To a solution of compound 67-3 (260 mg, 784.73 μmol, 1 eq) in DCM (6 mL) was added DMP (399.41 mg, 941.68 μmol, 291.54 uL, 1.2 eq). The reaction mixture was stirred at 20° C. for 2 h. The reaction mixture was quenched by NaHCO₃ (aq.) (20 mL) and extracted with DCM (20 mL×3). The combined organic layers were washed with brine (20 mL) and concentrated under reduced pressure to give compound 67-4 (420 mg, crude) as a yellow solid. LCMS: t_{R}=0.323 min, MS (ESI+) m/z=329.8[M+1]⁺. ¹H NMR (400 MHz, DMSO-d₆): δ=11.15 (s, 1H), 9.78 (s, 1H), 7.94-7.87 (m, 1H), 7.21 (d, J=8.4 Hz, 1H), 7.13-7.09 (m, 1H), 6.73-6.63 (m, 1H), 5.10 (dd, J=12.8, 5.2 Hz, 1H), 3.70-3.64 (m, 2H), 2.88-2.83 (m, 2H), 2.69-2.62 (m, 2H), 2.15-2.03 (m, 2H).

Procedure for preparation of MS-6-007. To a solution of compound 67-4 (131.66 mg, 255.87 μmol, 64% purity, 2 eq) and compound 624-5 (60 mg, 127.94 μmol, 1 eq, HCl) in THE (6 mL) was added HOAc (20.43 mg, 340.15 μmol, 19.45 uL, 2.66 eq), and the reaction mixture was stirred at 0° C. for 30 mins. Then NaBH(OAc)₃ (93.96 mg, 443.35 μmol, 3.47 eq) was added to the mixture, and the reaction mixture was stirred at 0° C. for 2 h. The reaction mixture was quenched by addition H₂O (1 mL) and were under reduced pressure to give a residue. The residue was purified by prep-HPLC (42%-72% ACN in water (10 mM NH₄HCO₃), 8 min) to give compound 67-MS-6-007 (12.07 mg, 15.86 μmol, 12.40% yield, 98% purity) as a yellow solid. LCMS: t_{R}=0.396 min, MS (ESI+) m/z=746.2[M+1]⁺. ¹H NMR (400 MHz, DMSO-d₆): δ=11.05 (d, J=0.8 Hz, 1H), 8.67 (s, 1H), 8.54 (dd, J=8.4, 3.2 Hz, 1H), 8.09-7.92 (m, 2H), 7.27-7.14 (m, 1H), 6.88 (d, J=8.8 Hz, 1H), 6.79-6.72 (m, 1H), 6.57 (s, 1H), 6.49-6.43 (m, 1H), 5.21-4.90 (m, 4H), 4.67-4.53 (m, 2H), 4.27-4.10 (m, 1H), 3.69 (s, 2H), 3.57-3.49 (m, 1H), 3.28-3.22 (m, 2H), 2.98-2.81 (m, 1H), 2.63 (dd, J=4.4, 2.4 Hz, 2H), 2.29 (s, 3H), 2.16-1.91 (m, 6H), 1.84-1.77 (m, 2H), 1.70 (d, J=5.2 Hz, 1H), 1.47 (t, J=7.2 Hz, 3H), 1.19 (d, J=5.6 Hz, 3H).

### Example 39

### Synthesis of MS-6-008

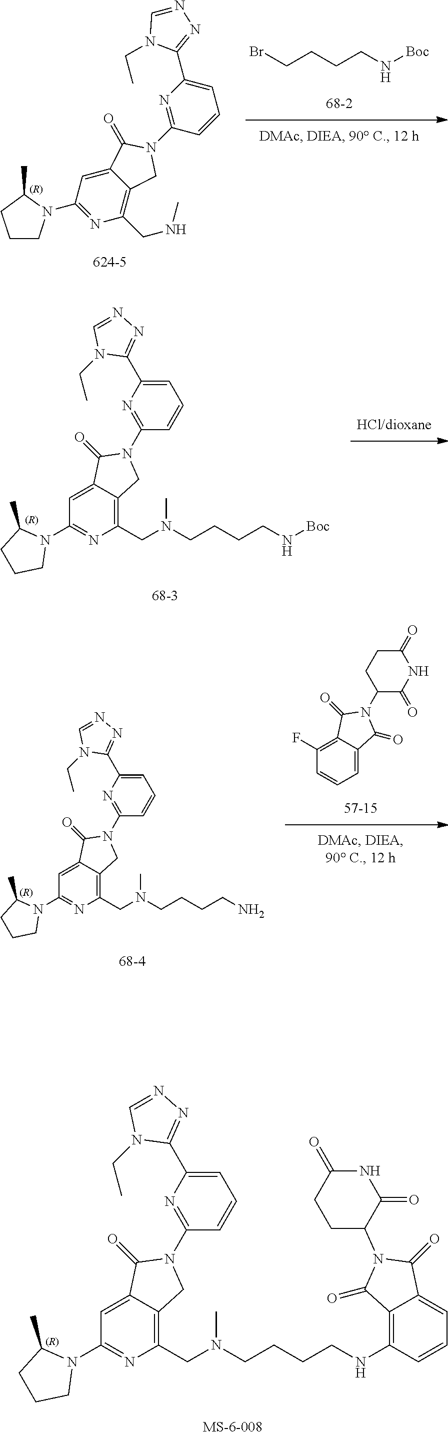

The procedure for preparing compound 624-5 is described in the synthesis of MS-6-027. Procedure for preparation of compound 68-3. To a mixture of compound 624-5 (200 mg, 426.46 μmol, 1 eq, HCl) in DMAc (3 mL) was added DIEA (165.35 mg, 1.28 mmol, 222.84 uL, 3 eq) and compound 68-2 (129.04 mg, 511.75 μmol, 104.91 uL, 1.2 eq) and the mixture was stirred at 90° C. for 12 h. The reaction mixture was filtered and purified by prep-HPLC (23%-53% ACN in water (0.225% FA), 58 min) to give compound 68-3 (35 mg, 57.97 μmol, 13.59% yield) as a brown solid. LCMS: t_{R}=0.417 min, MS (ESI+) m/z=604.4[M+1]⁺

Procedure for preparation of compound 68-4. To a solution of compound 68-3 (20 mg, 33.13 μmol, 1 eq) in DCM (2 mL) was added HCl/dioxane (4 M, 0.5 mL, 60.38 eq) and the reaction was stirred at 25° C. for 30 min. The reaction mixture was concentrated under reduced pressure to give compound 68-4 (30 mg, crude) as a yellow solid. LCMS: t_{R}=0.329 min, MS (ESI+) m/z=504.4[M+1]⁺

Procedure for preparation of MS-6-008. To a solution of 4 compound 68-4 (30 mg, 59.57 μmol, 1 eq) in DMAC (1 mL) was added DIEA (7.70 mg, 59.57 μmol, 10.38 uL, 1 eq) and compound 68-5 (16.45 mg, 59.57 μmol, 1 eq) and the mixture was stirred at 90° C. for 12 h. The reaction mixture was filtered and purified by prep-HPLC (15%-45% ACN in water (0.225% FA), 8 min) to give MS-6-008 (2 mg, 2.48 μmol, 4.17% yield, 100% purity, FA) as a yellow solid. LCMS: t_{R}=0.404 min, MS (ESI+) m/z=760.3 [M+1]⁺. ¹H NMR (400 MHz, DMSO-d₆): δ=11.08 (s, 1H), 8.68 (s, 1H), 8.58 (d, J=7.6 Hz, 1H), 8.43 (s, 1H), 8.07-8.03 (m, 1H), 8.00-7.97 (m, 1H), 7.47-7.40 (m, 1H), 6.95 (d, J=8.4 Hz, 1H), 6.91 (d, J=7.2 Hz, 1H), 6.60 (s, 1H), 6.43 (t, J=6.0 Hz, 1H), 5.11 (s, 2H), 4.97 (dd, J=5.2, 12.8 Hz, 1H), 4.61 (q, J=7.2 Hz, 2H), 4.27-4.14 (m, 1H), 3.68 (s, 2H), 2.90-2.82 (m, 1H), 2.60 (d, J=2.4 Hz, 2H), 2.43 (d, J=6.0 Hz, 4H), 2.24 (s, 3H), 2.09-1.92 (m, 6H), 1.72-1.66 (m, 1H), 1.56 (s, 4H), 1.47 (t, J=7.2 Hz, 3H), 1.19 (d, J=6.0 Hz, 3H).

### Example 40

### Synthesis of MS-6-009

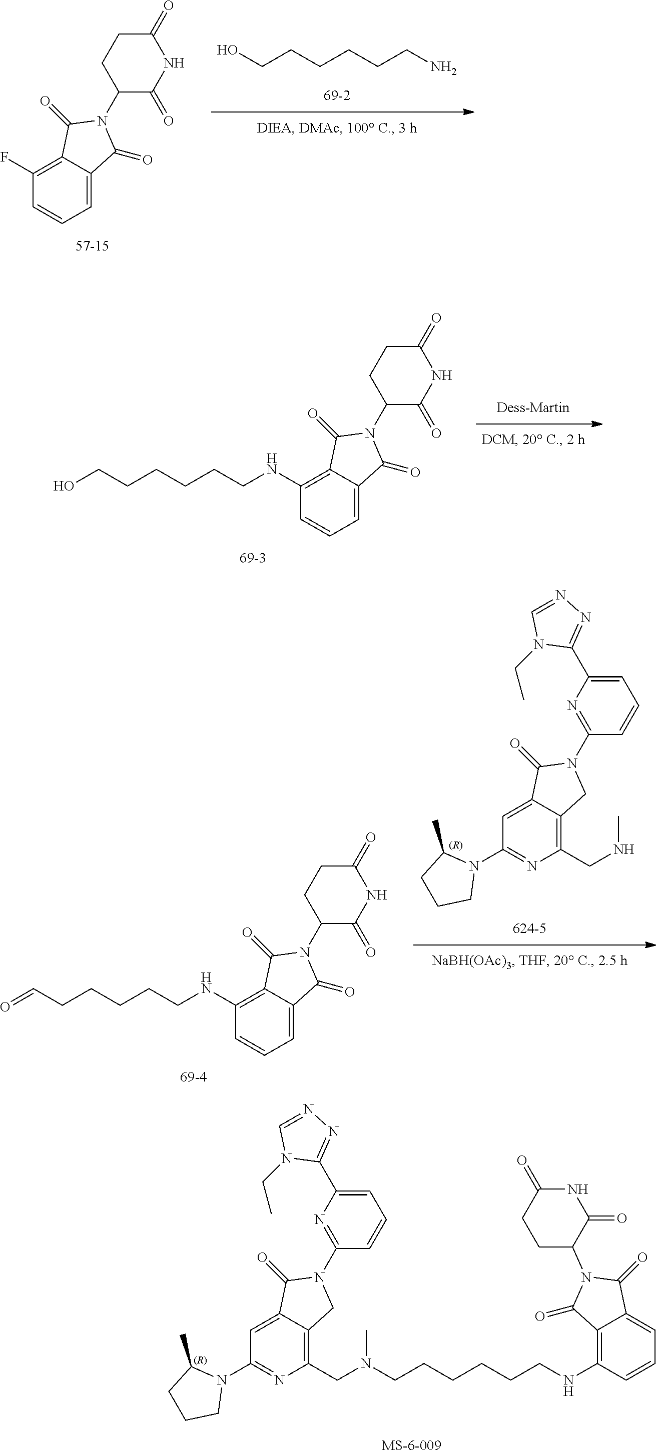

The procedure for preparing compound 624-5 is described in the synthesis of MS-6-027.

Procedure for preparation of compound 69-3. To a solution of compound 57-15 (commercially available, 1 g, 3.62 mmol, 1 eq) and compound 69-2 (636.39 mg, 5.43 mmol, 1.5 eq) in DMAc (10 mL) was added DIEA (1.40 g, 10.86 mmol, 1.89 mL, 3 eq) and the mixture was stirred at 100° C. for 3 h. The reaction mixture was quenched by addition H₂O (20 mL) and and extracted with EtOAc (20 mL×3). The combined organic layers were concentrated under reduced pressure to give compound 69-3 (1.110 g, crude) as a green oil. LCMS (EC6736-11-P1B): t_{R}=0.393 min, MS (ESI+) m/z=374.0[M+1]⁺. ¹H NMR (EC6736-11-P1B) (400 MHz, DMSO-d₆): δ=11.10 (s, 1H), 7.58 (dd, J=8.4, 7.2 Hz, 1H), 7.10 (d, J=8.4 Hz, 1H), 7.02 (d, J=7.2 Hz, 1H), 6.55 (t, J=6.0 Hz, 1H), 5.05 (dd, J=12.8, 5.6 Hz, 1H), 4.45 (t, J=5.4 Hz, 1H), 3.47-3.35 (m, 4H), 3.33-3.28 (m, 2H), 2.91-2.83 (m, 1H), 2.64-2.56 (m, 1H), 2.50-2.46 (m, 1H), 2.08-1.99 (m, 1H), 1.64-1.47 (m, 6H).

Procedure for preparation of compound 69-4. To a solution of compound 69-3 (400 mg, 1.07 mmol, 1 eq) in DCM (6 mL) was added DMP (545.23 mg, 1.29 mmol, 397.97 uL, 1.2 eq), the reaction mixture was stirred at 20° C. for 2 h. The reaction mixture was quenched by NaHCO₃ (aq.) (20 mL) and extracted with DCM (20 mL×3). The combined organic layers were washed with brine (20 mL) and concentrated under reduced pressure to give a residue. The residue was purified prep-TLC (SiO₂, PE:EtOAc=1:4) to give compound 69-4 (240 mg, 646.23 μmol, 60.33% yield) as a yellow oil. LCMS: t_{R}=0.400 min, MS (ESI+) m/z=353.9[M+1]⁺. ¹H NMR (400 MHz, DMSO-d₆): δ=11.09 (s, 1H), 9.67 (s, 1H), 7.58 (t, J=7.6 Hz, 1H), 7.10 (d, J=8.4 Hz, 1H), 7.02 (d, J=7.2 Hz, 1H), 6.54 (t, J=5.6 Hz, 1H), 5.05 (dd, J=5.2, 12.8 Hz, 1H), 2.94-2.83 (m, 1H), 2.65-2.57 (m, 2H), 2.45 (t, J=7.2 Hz, 2H), 2.04 (d, J=5.2 Hz, 1H), 1.64-1.54 (m, 5H), 1.40-1.30 (m, 3H).

Procedure for preparation of MS-6-009. To a solution of compound 624-5 (70 mg, 119.41 μmol, 80% purity, 1 eq, HCl) in THE (3 mL) was added TEA (24.17 mg, 238.82 μmol, 33.24 uL, 2 eq) and stirred for 10 min, then compound 69-4 (110.87 mg, 238.82 μmol, 80% purity, 2 eq) was added and stirred at 20° C. for 20 min, then NaBH(OAc)₃ (32.90 mg, 155.23 μmol, 1.3 eq) was added stirred at 20° C. for another 2 h. The reaction was quenched with H₂O (0.5 mL) and concentrated under reduced pressure to give a residue. The residue was purified by prep-HPLC (25%-55% ACN in water (0.225% FA), 10 min) to give MS-6-009 (21.11 mg, 26.36 μmol, 22.07% yield, 98.38% purity) as a yellow solid. LCMS: t_{R}=0.431 min, MS (ESI+) m/z=788.6[M+1]⁺. ¹H NMR (400 MHz, DMSO-d₆): δ=11.09 (s, 1H), 8.73 (s, 1H), 8.61 (d, J=8.4 Hz, 1H), 8.13-8.03 (m, 1H), 8.01-7.96 (m, 1H), 7.51 (t, J=7.8 Hz, 1H), 7.04-6.93 (m, 2H), 6.59 (s, 1H), 6.44 (t, J=5.6 Hz, 1H), 5.13 (s, 2H), 5.03 (dd, J=12.8, 5.6 Hz, 1H), 4.64 (q, J=7.2 Hz, 2H), 4.20 (t, J=5.6 Hz, 1H), 3.75-3.59 (m, 2H), 3.53 (t, J=7.2 Hz, 1H), 3.29-3.26 (m, 1H), 3.18 (q, J=6.4 Hz, 2H), 2.96-2.80 (m, 1H), 2.63-2.53 (m, 2H), 2.40-2.34 (m, 2H), 2.22 (s, 3H), 2.11-1.91 (m, 4H), 1.69 (d, J=1.6 Hz, 1H), 1.48 (t, J=7.2 Hz, 7H), 1.27 (s, 4H), 1.19 (d, J=6.0 Hz, 3H).

### Example 41

### Synthesis of MS-6-013

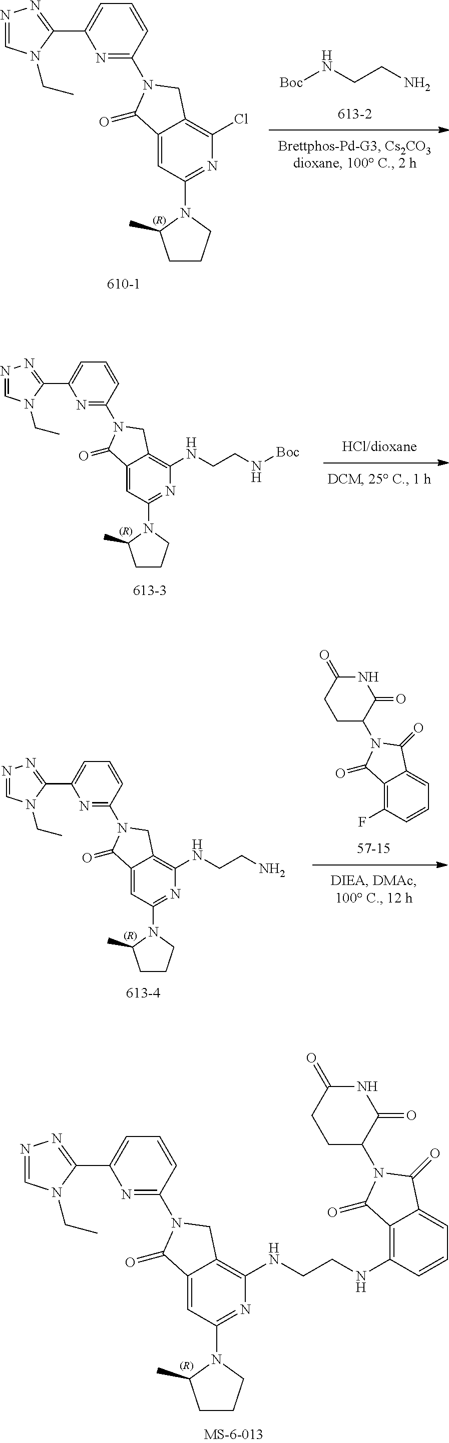

The procedure for preparing compound 610-1 is described in the synthesis of MS-6-006.

Procedure for preparation of compound 613-3. A mixture of compound 610-1 (500 mg, 1.18 mmol, 1 eq), compound 613-2 (566.93 mg, 3.54 mmol, 555.81 uL, 3 eq), BrettPhos Pd G₃ (213.85 mg, 235.91 μmol, 0.2 eq) and Cs₂CO₃ (1.15 g, 3.54 mmol, 3 eq) in dioxane (10 mL) was degassed and purged with N₂ for 3 times. The mixture was stirred at 100° C. for 2 h. The reaction mixture was filtered and purified by prep-HPLC (50%-80% ACN in water (0.225% FA), 10 min) to give compound 613-3 (50 mg, 91.30 μmol, 7.74% yield) as a brown solid. LCMS: t_{R}=0.450 min, MS (ESI+) m/z=548.2[M+1]⁺. ¹H NMR (400 MHz, DMSO-d₆): δ=8.76 (s, 1H), 8.59 (d, J=8.4 Hz, 1H), 8.18 (s, 1H), 8.10-8.03 (m, 1H), 7.94 (d, J=7.6 Hz, 1H), 6.93 (t, J=4.8 Hz, 1H), 6.65 (t, J=5.2 Hz, 1H), 5.88 (s, 1H), 4.82 (s, 2H), 4.68-4.59 (m, 2H), 4.19 (t, J=6.0 Hz, 1H), 3.47 (s, 2H), 3.22 (d, J=5.6 Hz, 2H), 2.11-1.87 (m, 5H), 1.68 (d, J=5.6 Hz, 1H), 1.41-1.36 (m, 12H), 1.20 (d, J=6.4 Hz, 3H).

Procedure for preparation of compound 613-4. To a solution of compound 613-3 (50 mg, 91.30 μmol, 1 eq) in DCM (1 mL) was added HCl/dioxane (4 M, 540.22 uL, 23.67 eq) and the mixture was stirred at 25° C. for 1 h. The reaction mixture was concentrated under reduced pressure to give compound 613-4 (50 mg, crude, HCl) as a brown solid, used directly. LCMS: t_{R}=0.379 min, MS (ESI+) m/z=447.9[M+1]⁺

Procedure for preparation of MS-6-013. To a solution of compound 613-4 (50 mg, 103.31 μmol, 1 eq, HCl) in DMAc (1 mL) was added DIEA (40.05 mg, 309.92 μmol, 53.98 uL, 3 eq) and compound 57-15 (28.54 mg, 103.31 μmol, 1 eq) and the mixture was stirred at 100° C. for 12 h. The reaction mixture was filtered and purified by prep-HPLC (46%-76% ACN in water (0.225% FA), 10 min) to give MS-6-013 (9.95 mg, 13.27 μmol, 12.85% yield, 100% purity, FA) as a yellow solid. LCMS: t_{R}=0.451 min, MS (ESI+) m/z=704.3 [M+1]⁺. ¹H NMR (400 MHz, DMSO-d₆): δ=11.27-10.92 (m, 1H), 8.72 (s, 1H), 8.58 (d, J=8.4 Hz, 1H), 8.07 (t, J=8.0 Hz, 1H), 7.93 (d, J=7.6 Hz, 1H), 7.54 (t, J=7.6 Hz, 1H), 7.15 (dd, J=8.4, 5.4 Hz, 1H), 7.01 (d, J=7.2 Hz, 1H), 6.87-6.73 (m, 2H), 5.89 (s, 1H), 5.02 (dd, J=12.8, 5.2 Hz, 1H), 4.79 (s, 2H), 4.60 (q, J=7.2 Hz, 2H), 4.25-4.06 (m, 1H), 3.80-3.68 (m, 1H), 3.68-3.57 (m, 3H), 3.47 (, J=7.8 Hz, 2H), 2.92-2.82 (m, 1H), 2.64-2.53 (m, 2H), 2.05-1.87 (m, 4H), 1.66 (s, 1H), 1.35 (t, J=7.2 Hz, 3H), 1.18 (d, J=6.0 Hz, 3H).

### Example 42

### Synthesis of MS-6-014

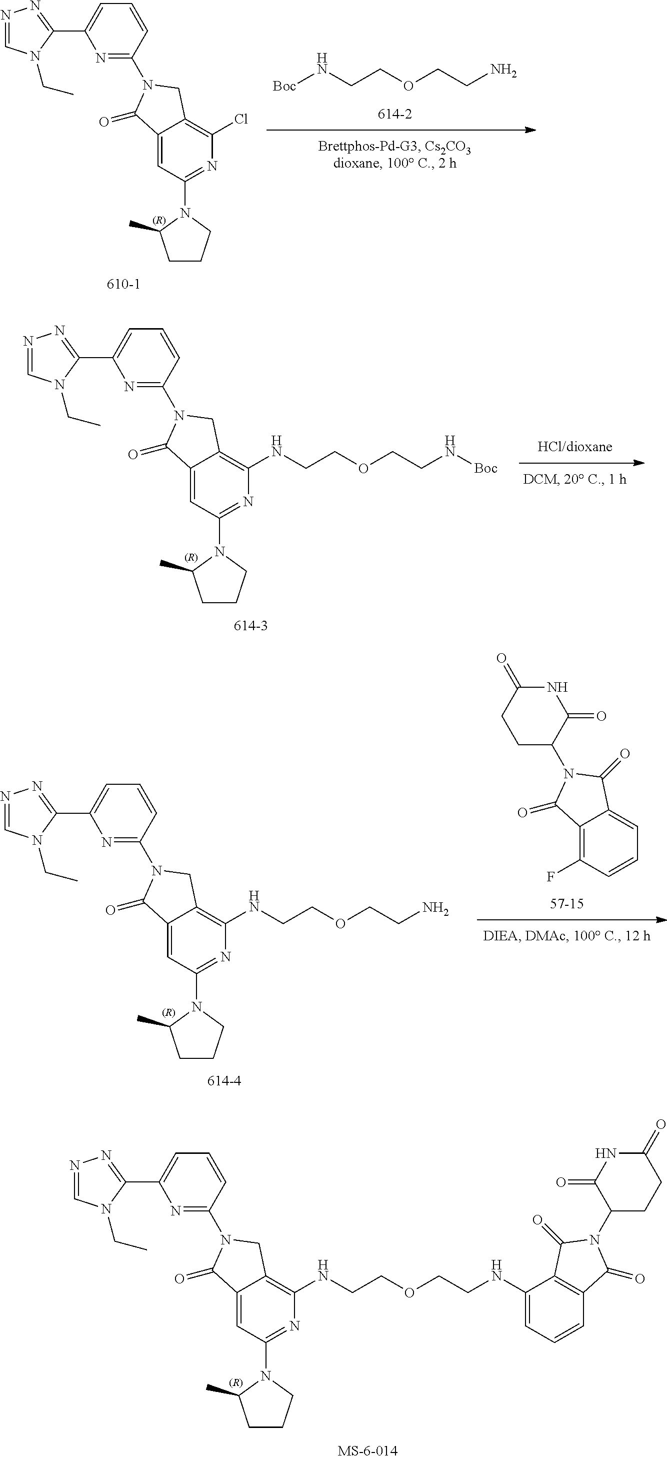

The procedure for preparing compound 610-1 is described in the synthesis of MS-6-006.

Procedure for preparation of compound 614-3. A mixture of 610-1 (500 mg, 1.18 mmol, 1 eq), compound 614-2 (602.34 mg, 2.95 mmol, 2.5 eq), BrettPhos Pd G3 (106.92 mg, 117.95 μmol, 0.1 eq) and Cs₂CO₃ (768.63 mg, 2.36 mmol, 2 eq) in dioxane (10 mL) was degassed and purged with N₂ for 3 times and the mixture was stirred at 100° C. for 2 h. The reaction mixture was filtered and purified by prep-HPLC (40%-70% ACN in water (0.225% FA), 10 min) to give compound 614-3 (50 mg, 84.50 μmol, 7.16% yield) as a brown solid. LCMS: t_{R}=0.453 min, MS (ESI+) m/z=592.1[M+1]⁺. ¹H NMR (400 MHz, DMSO-d₆): δ=8.75 (s, 1H), 8.59 (d, J=8.0 Hz, 1H), 8.06 (t, J=8.0 Hz, 1H), 7.93 (d, J=7.6 Hz, 1H), 6.84-6.65 (m, 2H), 5.88 (s, 1H), 4.83 (s, 2H), 4.64 (q, J=7.2 Hz, 2H), 4.17 (t, J=6.4 Hz, 1H), 4.03 (q, J=7.2 Hz, 1H), 3.68-3.52 (m, 4H), 3.13-3.05 (m, 2H), 2.08-1.91 (m, 5H), 1.74-1.63 (m, 1H), 1.41-1.33 (m, 13H), 1.23-1.17 (m, 4H).

Procedure for preparation of compound 614-4. To a solution of compound 614-3 (50 mg, 84.50 μmol, 1 eq) in DCM (1 mL) was added HCl/dioxane (4 M, 0.5 mL, 23.67 eq) and the mixture was stirred at 20° C. for 1 h. The reaction mixture was concentrated to give compound 614-4 (50 mg, crude, HCl) as a yellow solid, which was used directly. LCMS: t_{R}=0.343 min, MS (ESI+) m/z=492.0[M+1]

Procedure for preparation of MS-6-014. To a solution of compound 614-4 (50 mg, 94.69 μmol, 1 eq, HCl) in DMAc (0.5 mL) was added DIEA (36.71 mg, 284.06 μmol, 49.48 uL, 3 eq) and compound 57-15 (26.15 mg, 94.69 μmol, 1 eq), the mixture was stirred at 100° C. for 12 h. The reaction mixture was filtered and purified by prep-HPLC (31%-61% ACN in water (0.225% FA), 10 min) to give MS-6-014 (7.04 mg, 8.41 μmol, 8.89% yield, 94.88% purity, FA) as a yellow solid. LCMS: t_{R}=0.680 min, MS (ESI+) m/z=748.2 [M+1]⁺. ¹H NMR (400 MHz, DMSO-d₆): δ=11.26-10.93 (m, 1H), 8.73 (s, 1H), 8.58 (d, J=8.4 Hz, 1H), 8.32-8.27 (m, 1H), 8.06 (t, J=8.0 Hz, 1H), 7.94 (d, J=7.2 Hz, 1H), 7.62-7.50 (m, 1H), 7.13 (d, J=8.6 Hz, 1H), 7.02 (d, J=7.2 Hz, 1H), 6.72 (t, J=5.4 Hz, 1H), 6.61 (t, J=5.4 Hz, 1H), 5.86 (s, 1H), 5.01 (dd, J=12.8, 5.2 Hz, 1H), 4.81 (s, 2H), 4.62 (q, J=7.2 Hz, 2H), 4.19-4.08 (m, 1H), 3.73-3.58 (m, 6H), 3.54-3.42 (m, 5H), 2.89-2.78 (m, 1H), 2.57 (d, J=4.0 Hz, 1H), 2.06-1.88 (m, 4H), 1.69-1.58 (m, 1H), 1.37 (t, J=7.2 Hz, 3H), 1.17 (d, J=6.0 Hz, 3H).

### Example 43

### Synthesis of MS-6-015

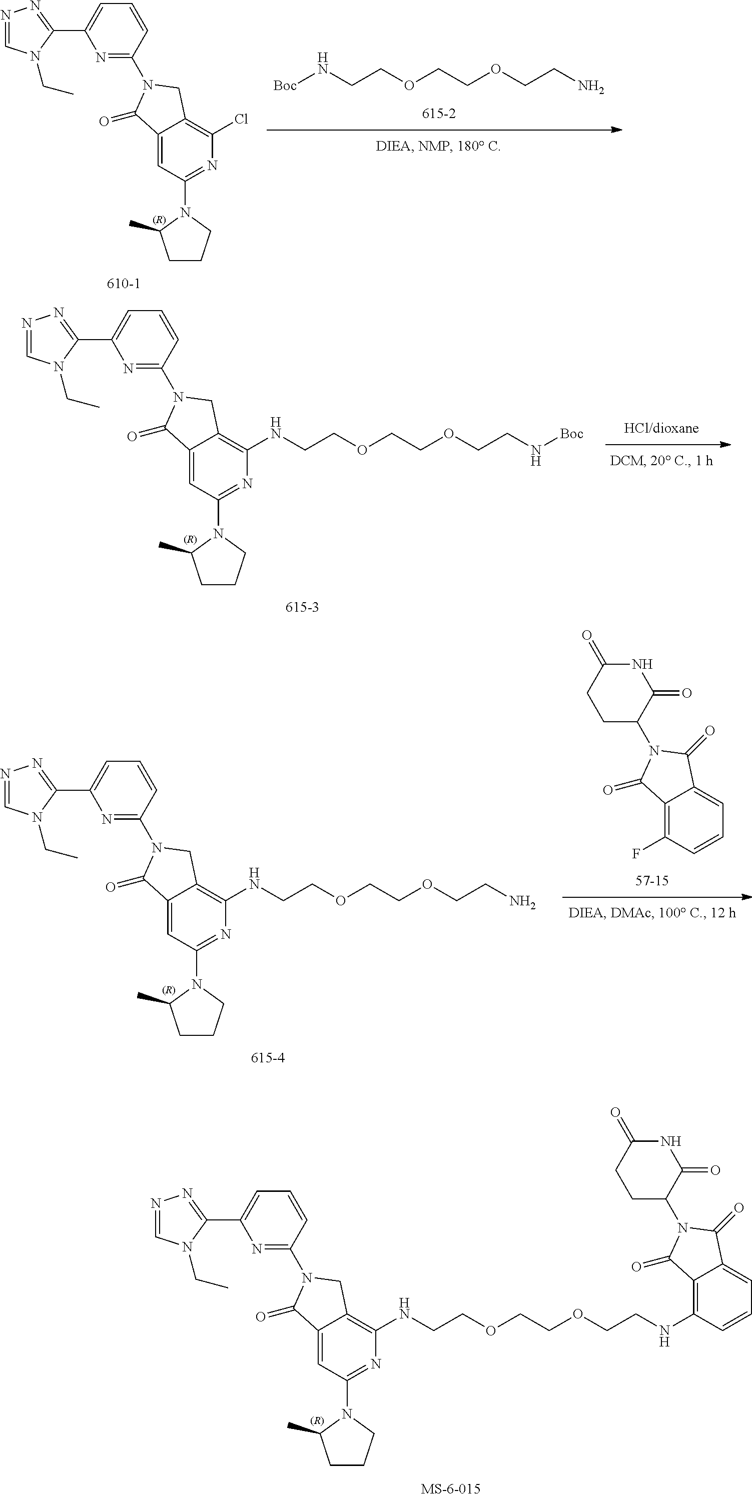

The procedure for preparing compound 610-1 is described in the synthesis of MS-6-006.

Procedure for preparation of compound 615-3. A mixture of 610-1 (400 mg, 943.62 μmol, 1 eq) compound 615-2 (1.17 g, 4.72 mmol, 5 eq) and DIEA (365.87 mg, 2.83 mmol, 493.09 uL, 3 eq) in NMP (4 mL) was microwaved under 160° C. for 2 h. The reaction mixture was quenched by addition H₂O (5 mL) and extracted with (50×3 mL), the combined organic layers was washed with brine (80 mL) and concentrated under reduced pressure to give a residue. The residue was purified by Prep-HPLC (41%-71% ACN in water (0.225% FA), 10 min) to give compound 615-3 (40 mg, 62.92 μmol, 6.67% yield) as a brown solid. LCMS: t_{R}=0.436 min, MS (ESI+) m/z=636.4 [M+1]⁺. ¹H NMR (400 MHz, DMSO-d₆): δ=8.77-8.74 (m, 1H), 8.59 (d, J=8.1 Hz, 1H), 8.06 (t, J=8.0 Hz, 1H), 7.94 (d, J=7.2 Hz, 1H), 6.85-6.68 (m, 2H), 5.87 (s, 1H), 4.82 (s, 2H), 4.65 (q, J=7.2 Hz, 2H), 4.21-4.14 (m, 1H), 3.67-3.49 (m, 8H), 3.46 (d, J=9.6 Hz, 8H), 3.05 (q, J=6.0 Hz, 2H), 2.14-1.88 (m, 3H), 1.72-1.63 (m, 1H), 1.44-1.30 (m, 12H), 1.20 (d, J=6.4 Hz, 3H).

Procedure for preparation of compound 615-4. To a solution of compound 615-3 (100 mg, 157.29 μmol, 1 eq) in DCM (1 mL) was added HCl/dioxane (4 M, 0.5 mL, 12.72 eq) and the mixture was stirred at 25° C. for 12 h. The reaction mixture was concentrated to give compound 615-4 (100 mg, crude, HCl) as a yellow solid, which was used directly. LCMS: t_{R}=0.341 min, MS (ESI+) m/z=536.2 [M+1]⁺

Procedure for preparation of MS-6-015. To a solution of compound 615-4 (100 mg, 174.79 μmol, 1 eq, HCl) in DMAc (1 mL) was added DIEA (67.77 mg, 524.38 μmol, 91.34 uL, 3 eq) and compound 57-15 (48.28 mg, 174.79 μmol, 1 eq) and the mixture was stirred at 100° C. for 12 h. The reaction mixture was filtered and purified by prep-HPLC (40%-70% ACN in water (10 mM NH₄HCO₃), 8 min) to give MS-6-015 (6.49 mg, 7.96 μmol, 4.55% yield, 97.08% purity) as a yellow solid. LCMS: t_{R}=0.402 min, MS (ESI+) m/z=792.5 [M+1]⁺. ¹H NMR (400 MHz, DMSO-d₆): δ=11.17-10.97 (m, 1H), 8.73 (s, 1H), 8.58 (d, J=8.4 Hz, 1H), 8.11-8.01 (m, 1H), 7.93 (dd, J=7.6, 0.8 Hz, 1H), 7.58-7.48 (m, 1H), 7.10 (d, J=8.4 Hz, 1H), 7.01 (d, J=7.2 Hz, 1H), 6.69 (t, J=5.6 Hz, 1H), 6.59 (t, J=6.0 Hz, 1H), 5.86 (s, 1H), 5.03 (dd, J=12.8, 5.2 Hz, 1H), 4.81 (s, 2H), 4.63 (q, J=7.2 Hz, 2H), 4.20-4.09 (m, 1H), 3.63 (dd, J=12.0, 6.0 Hz, 6H), 3.58-3.53 (m, 2H), 3.49-3.41 (m, 6H), 2.90-2.81 (m, 1H), 2.60 (s, 2H), 2.05-1.89 (m, 4H), 1.64 (dd, J=7.6, 1.2 Hz, 1H), 1.38 (t, J=7.2 Hz, 3H), 1.18 (d, J=6.4 Hz, 3H).

### Example 44

### Synthesis of MS-6-016

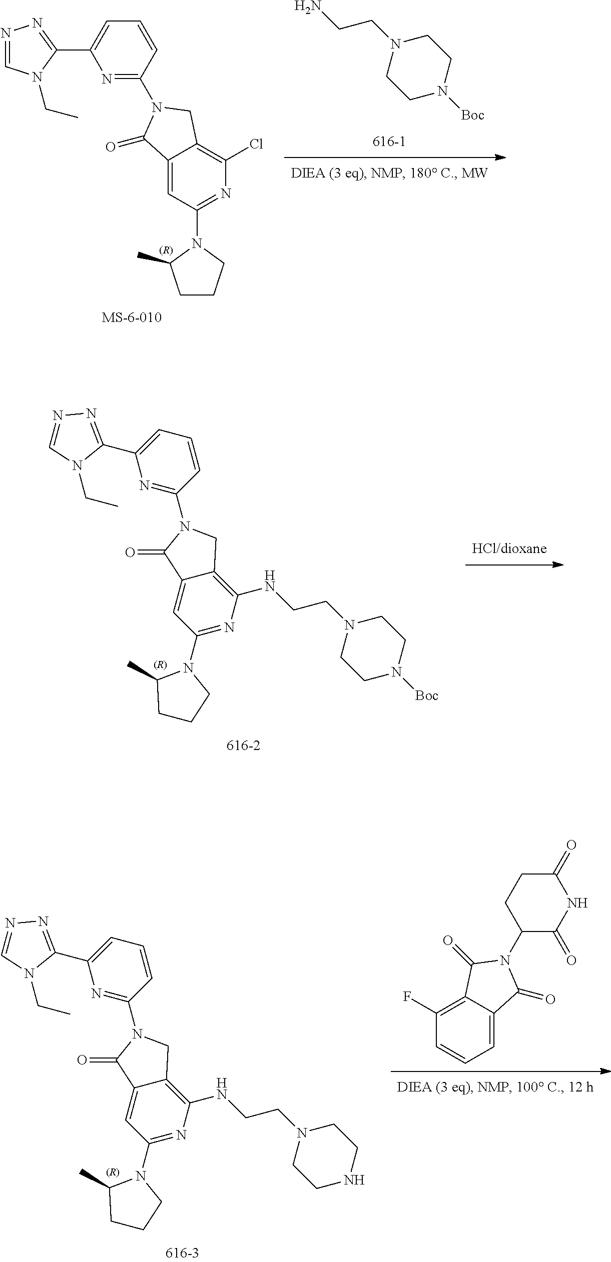

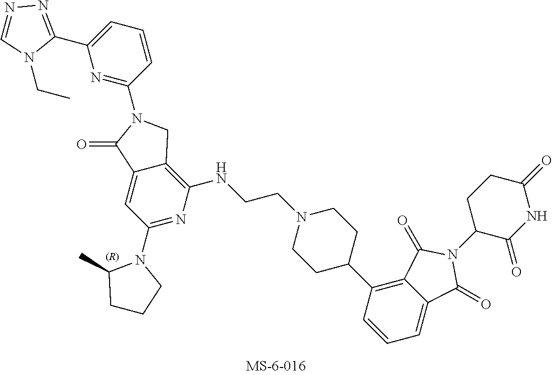

The procedure for preparing compound 610-1 is described in the synthesis of MS-6-006.

Procedure for preparation of compound 616-2. A mixture of compound 610-1 (500 mg, 1.18 mmol, 1 eq), compound 616-1 (811.46 mg, 3.54 mmol, 3 eq) and DIEA (457.34 mg, 3.54 mmol, 616.36 uL, 3 eq) in NMP (3 mL) was microwaved under 150° C. for 2 h. The reaction mixture was filtered and purified by Prep-HPLC (0%-50% ACN in water (0.225% FA), 10 min) to give compound 616-2 (180 mg, 291.85 μmol, 24.74% yield) as a brown solid. LCMS: t_{R}=0.429 min, MS (ESI+) m/z=617.2[M+1]⁺. ¹H NMR (400 MHz, DMSO-d₆): δ=8.75 (s, 1H), 8.58 (d, J=8.4 Hz, 1H), 8.05 (t, J=8.0 Hz, 1H), 7.93 (d, J=7.6 Hz, 1H), 6.51 (t, J=5.2 Hz, 1H), 5.86 (s, 1H), 4.81 (s, 2H), 4.63 (q, J=7.2 Hz, 2H), 4.18 (t, J=6.0 Hz, 1H), 3.57-3.47 (m, 4H), 3.35-3.31 (m, 4H), 2.59 (t, J=7.2 Hz, 2H), 2.42 (t, J=5.2 Hz, 4H), 2.07-1.91 (m, 3H), 1.66 (d, J=2.8 Hz, 1H), 1.43-1.38 (m, 11H), 1.20 (d, J=6.4 Hz, 3H).

Procedure for preparation of compound 616-3. To a solution of compound 616-2 (120 mg, 194.57 μmol, 1 eq) in DCM (2 mL) was added HCl/dioxane (4 M, 1 mL, 20.56 eq) and the mixture was stirred at 25° C. for 1 h. The reaction mixture was concentrated under reduced pressure to give compound 616-3 (110 mg, crude, HCl) as a yellow solid, used directly. LCMS: t_{R}=0.336 min, MS (ESI+) m/z=517.0[M+1]⁺

Procedure for preparation of compound MS-6-016. To a solution of compound 616-3 (100 mg, 180.80 μmol, 1 eq, HCl) and 2-(2,6-dioxopiperidin-3-yl)-4-fluoroisoindoline-1,3-dione (49.94 mg, 180.80 μmol, 1 eq) in DMAc (1 mL) was added DIEA (116.83 mg, 903.99 μmol, 157.46 uL, 5 eq) and the mixture was stirred at 100° C. for 12 h. The reaction mixture was filtered and purified by prep-HPLC (22%-52% ACN in water (0.225% FA), 58 min) and prep-HPLC (37%-67% ACN in water (10 mM NH₄HCO₃), 8 min) to give MS-6-016 (17.33 mg, 22.10 μmol, 12.23% yield, 98.58% purity) as a yellow solid. LCMS: t_{R}=1.984 min, MS (ESI+) m/z=773.3[M+1]⁺. ¹H NMR (400 MHz, DMSO-d₆): δ=11.09 (s, 1H), 8.74 (s, 1H), 8.59 (d, J=8.4 Hz, 1H), 8.07 (t, J=8.0 Hz, 1H), 7.94 (d, J=7.6 Hz, 1H), 7.79-7.66 (m, 1H), 7.36 (t, J=7.2 Hz, 2H), 6.57 (t, J=5.2 Hz, 1H), 5.88 (s, 1H), 5.10 (dd, J=12.8, 5.2 Hz, 1H), 4.84 (s, 2H), 4.64 (q, J=7.2 Hz, 2H), 4.21 (t, J=5.6 Hz, 1H), 3.66-3.54 (m, 2H), 3.49 (t, J=8.0 Hz, 1H), 3.28 (d, J=9.6 Hz, 2H), 2.96-2.79 (m, 1H), 2.76-2.54 (m, 10H), 2.15-1.90 (m, 5H), 1.67 (s, 1H), 1.41 (t, J=7.2 Hz, 3H), 1.23 (d, J=6.4 Hz, 3H).

### Example 45

### Synthesis of MS-6-017

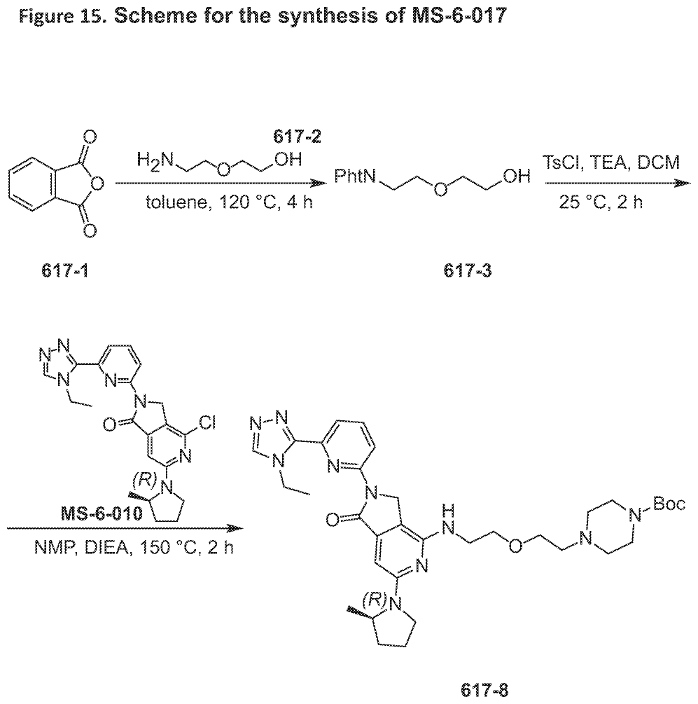

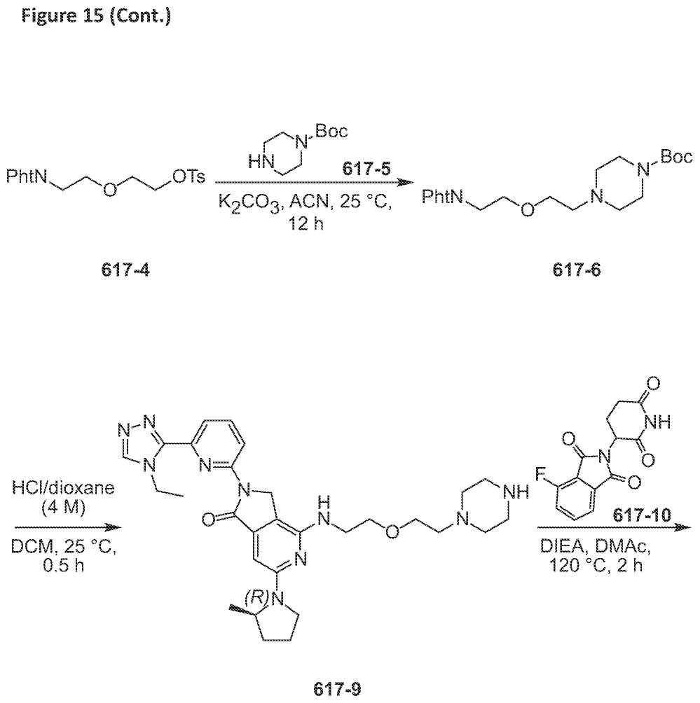

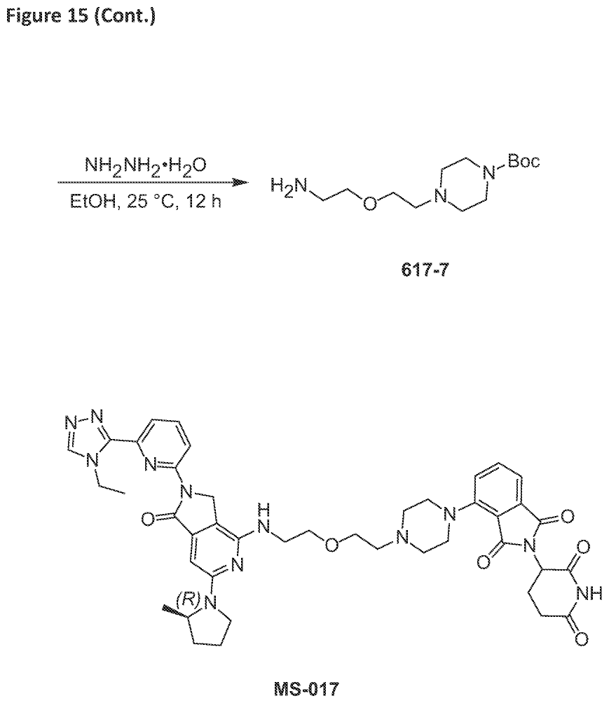

The reaction scheme for the synthesis of MS-6-017 is shown in FIG. 15.

The procedure for preparing compound 610-1 is described in the synthesis of MS-6-006.

Procedure for preparation of compound 617-3. A mixture of compound 617-1 (5 g, 33.76 mmol, 1 eq) and compound 617-2 (3.55 g, 33.76 mmol, 3.38 mL, 1 eq) in toluene (30 mL) was stirred at 120° C. for 4 h. The reaction mixture was concentrated under reduced pressure to give compound 617-3 (6 g, 25.51 mmol, 75.56% yield) as a yellow oil. ¹H NMR (400 MHz, DMSO-d₆): δ=7.90-7.79 (m, 4H), 4.67 (s, 1H), 3.79-3.70 (m, 2H), 3.66-3.59 (m, 2H), 3.55-3.49 (m, 1H), 3.48-3.43 (m, 3H).

Procedure for preparation of compound 617-4. To a solution of compound 617-3 (5 g, 21.26 mmol, 1 eq) in DCM (40 mL) was added TEA (6.45 g, 63.77 mmol, 8.88 mL, 3 eq) and TsCl (4.86 g, 25.51 mmol, 1.2 eq), the resulting mixture was stirred at 25° C. for 2 h. The reaction mixture was quenched by H₂O (50 mL) and extracted with DCM (50 mL×3), the combined organic layers were concentrated under reduced pressure to give a residue. The residue was purified by silica column chromatography (SiO₂, Petroleum ether/Ethyl acetate=100/1 to 3/1) to give compound 617-4 (7.5 g, 19.26 mmol, 90.61% yield) as a white solid. LCMS: t_{R}=0.667 min, MS (ESI+) m/z=390.0[M+1]⁺. ¹H NMR (400 MHz, DMSO-d₆): δ=7.96-7.80 (m, 4H), 7.71 (d, J=8.4 Hz, 2H), 7.42 (d, J=8.0 Hz, 2H), 4.06 (dd, J=5.2, 3.6 Hz, 2H), 3.77-3.64 (m, 2H), 3.63-3.51 (m, 4H), 2.44-2.33 (m, 3H).

Procedure for preparation of compound 617-6. To a solution of compound 617-4 (5 g, 12.84 mmol, 1 eq) and compound 617-5 (2.39 g, 12.84 mmol, 1 eq) in MeCN (50 mL) was added K₂CO₃ (3.55 g, 25.68 mmol, 2 eq) and the mixture was stirred at 25° C. for 12 h. The reaction was quenched by H₂O (50 mL) and extracted with DCM (100 mL), the organic layer was concentrated under reduced pressure to give a residue. The residue was purified by silica column chromatography (SiO₂, Petroleum ether/Ethyl acetate=100/1 to 0/1) to give compound 617-6 (5 g, 12.39 mmol, 96.52% yield) as a yellow solid. LCMS: t_{R}=0.618 min, MS (ESI+) m/z=404.2[M+1]⁺. ¹H NMR (400 MHz, CDCl₃): δ=7.78 (dd, J=5.2, 3.6 Hz, 2H), 7.69-7.64 (m, 2H), 3.86-3.81 (m, 2H), 3.77 (s, 2H), 3.68-3.63 (m, 2H), 3.53 (s, 4H), 3.03-2.50 (m, 6H), 1.39 (s, 9H)

Procedure for preparation of compound 617-7. To a solution of compound 617-6 (5 g, 12.39 mmol, 1 eq) in EtOH (50 mL) was added N₂H₄·H₂O (7.30 g, 123.92 mmol, 7.09 mL, 85% purity, eq) and the mixture was stirred at 25° C. for 12 h. The reaction mixture was filtered and concentrated under reduced pressure to give compound 617-7 (2.9 g, 10.61 mmol, 85.60% yield) as a yellow oil. LCMS: t_{R}=0.167 min, MS (ESI+) m/z=274.2[M+1]⁺. ¹H NMR (400 MHz, DMSO-d₆): δ=3.51-3.47 (m, 2H), 3.36-3.26 (m, 6H), 2.64 (t, J=5.6 Hz, 2H), 2.48 (t, J=6.0 Hz, 2H), 2.40-2.30 (m, 4H), 1.39 (s, 9H).

Procedure for preparation of compound 617-8. A mixture of compound 617-7 (1.03 g, 3.77 mmol, 2 eq), compound 610-1 (800 mg, 1.89 mmol, 1 eq) and DIEA (731.74 mg, 5.66 mmol, 986.17 uL, 3 eq) in NMP (4 mL) was microwaved under 150° C. for 2 h. The residue was purified by prep-HPLC (33%-63% ACN in water (0.05% HCl), 10 min) and lyophilized to give compound 617-8 (150 mg, 226.99 μmol, 12.03% yield) as a brown solid. LCMS: t_{R}=0.398 min, MS (ESI+) m/z=661.4[M+1]

Procedure for preparation of compound 617-9. To a solution of compound 617-8 (120 mg, 181.60 μmol, 1 eq) 120 mg, 181.60 μmol, 1 eq) in DCM (2 mL) was added HCl/dioxane (4 M, 22.70 uL, 0.5 eq) and the mixture was stirred at 25° C. for 0.5 h. The reaction mixture was quenched by NaHCO₃ to adjust pH to 9 and extracted with DCM (50 mL), the organic layer was concentrated under reduced pressure to give compound 617-9 (100 mg, 124.85 μmol, 68.75% yield, 70% purity) as a brown solid. LCMS: t_{R}=0.328 min, MS (ESI+) m/z=561.1[M+1]⁺

Procedure for preparation of MS-6-017. A mixture of compound 617-9 (100 mg, 124.85 μmol, 70% purity, 1 eq) and compound 617-10 (34.48 mg, 124.85 μmol, 1 eq) in DMAc (1 mL) was added DIEA (16.14 mg, 124.85 μmol, 21.75 uL, 1 eq) and the mixture was stirred at 120° C. for 2 h. The mixture was purified by prep-HPLC (17%-47% ACN in water (0.225% FA), 10 min) and lyophilized to give MS-6-017 (33.33 mg, 36.92 μmol, 29.58% yield, 95.6% purity, FA) as a yellow solid. LCMS: t_{R}=1.977 min, MS (ESI+) m/z=817.4[M+1]⁺. ¹H NMR (400 MHz, DMSO-d₆) δ=11.10 (s, 1H), 8.74 (s, 1H), 8.58 (d, J=8.4 Hz, 1H), 8.25 (s, 1H), 8.05 (t, J=8.0 Hz, 1H), 7.93 (d, J=7.6 Hz, 1H), 7.66 (t, J=7.6 Hz, 1H), 7.43-7.21 (m, 2H), 6.74 (t, J=5.4 Hz, 1H), 5.87 (s, 1H), 5.16-5.00 (m, 1H), 4.82 (s, 2H), 4.64 (q, J=7.2 Hz, 2H), 4.23-4.12 (m, 1H), 3.67-3.58 (m, 6H), 3.47 (d, J=1.2 Hz, 2H), 3.24 (s, 4H), 2.92-2.83 (m, 1H), 2.65-2.54 (m, 8H), 2.12-1.95 (m, 4H), 1.66 (d, J=2.0 Hz, 1H), 1.38 (t, J=7.2 Hz, 3H), 1.25-1.15 (m, 3H).

### Example 46

### Synthesis of MS-6-018

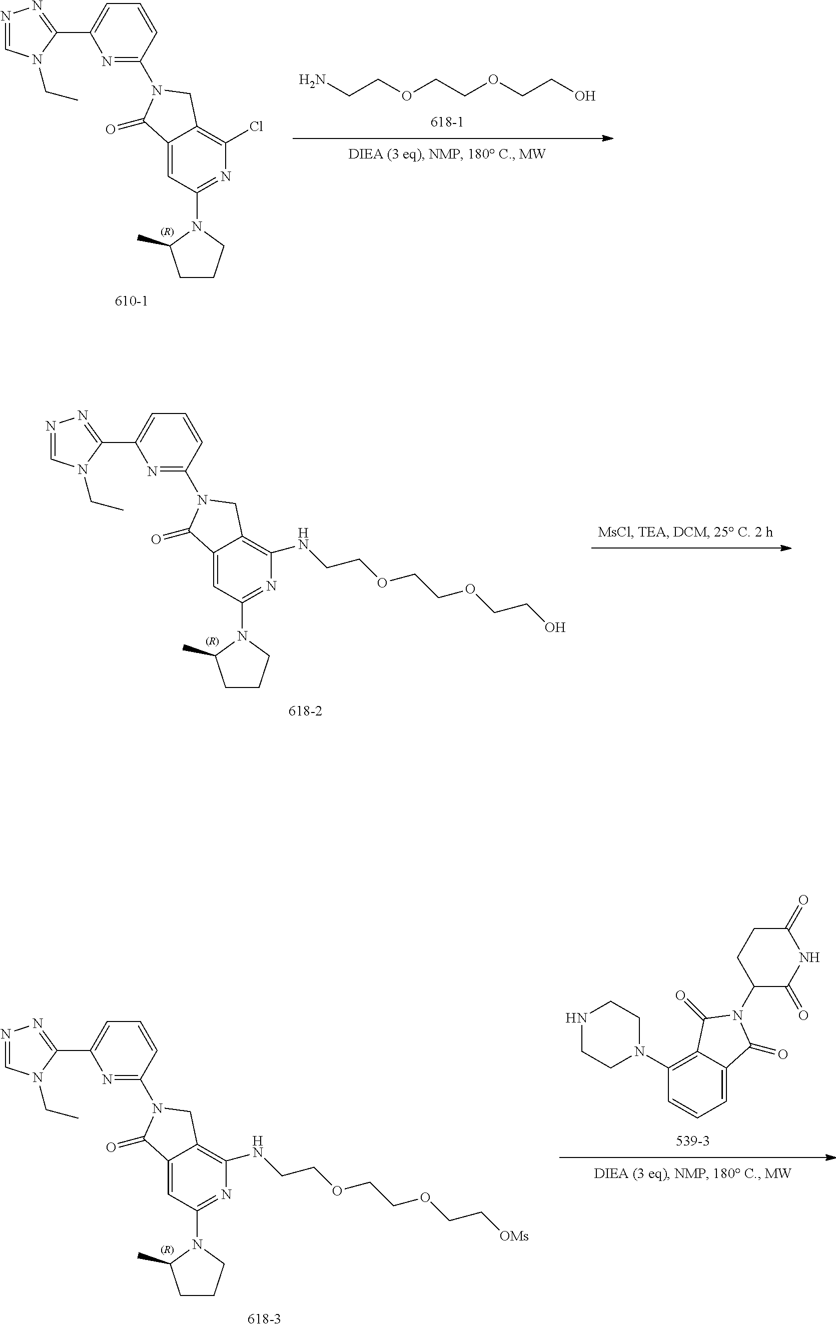

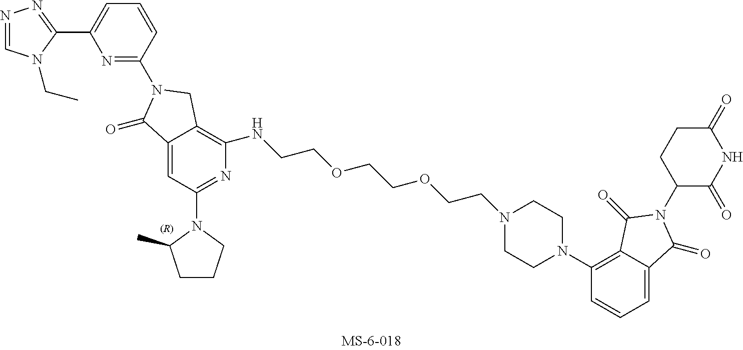

The procedure for preparing compound 610-1 is described in the synthesis of MS-6-006.

The procedure for preparing 539-3 is described in the synthesis of MS-5-039.

Procedure for preparation of compound 618-3. To a solution of compound 618-2 (100 mg, 186.35 μmol, 1 eq) in DCM (1 mL) was added MsCl (42.69 mg, 372.70 μmol, 28.85 uL, 2 eq), Et₃N (37.71 mg, 372.70 μmol, 51.88 uL, 2 eq) at 0° C. Then the mixture was stirred at 25° C. for 1 h. The mixture was concentrated to give compound 618-3 (114 mg, crude) as brown solid. LCMS: t_{R}=0.391 min, MS (ESI+) m/z=615.1[M+1]⁺

Procedure for preparation of compound MS-6-018. A flask charged with a stir bar was added compound 539-3 (114 mg, 185.45 μmol, 1 eq), compound 618-3 (105.38 mg, 278.18 μmol, 1.5 eq, HCl), DIEA (119.84 mg, 927.26 μmol, 161.51 uL, 5 eq) and NMP (1 mL). The mixture was stirred at 100° C. for 12 h. The mixture was filtered and purified by prep-HPLC (15%-45% ACN in water (0.225% FA), 8 min) to give MS-6-018 (12.62 mg, 12.80 μmol, 6.90% yield, 92% purity, FA) as yellow solid. LCMS: t_{R}=0.373 min, MS (ESI+) m/z=861.3[M+1]⁺. ¹H NMR (400 MHz, DMSO-d₆) δ=11.09 (s, 1H), 8.74 (s, 1H), 8.56 (d, J=8.0 Hz, 1H), 8.04 (t, J=8.0 Hz, 1H), 7.92 (d, J=7.6 Hz, 1H), 7.77-7.53 (m, 1H), 7.32 (d, J=7.2 Hz, 1H), 7.24 (d, J=8.6 Hz, 1H), 6.72 (t, J=5.4 Hz, 1H), 5.84 (s, 1H), 5.07 (dd, J=12.8, 5.4 Hz, 1H), 4.80 (s, 2H), 4.63 (q, J=7.2 Hz, 2H), 4.21-4.09 (m, 1H), 3.69-3.41 (m, 16H), 3.25 (s, 2H), 2.93-2.80 (m, 1H), 2.66-2.52 (m, 6H), 2.07-1.88 (m, 4H), 1.71-1.58 (m, 1H), 1.38 (t, J=7.2 Hz, 3H), 1.19 (d, J=6.0 Hz, 3H).

### Example 47 Synthetic Route for MS-6-019

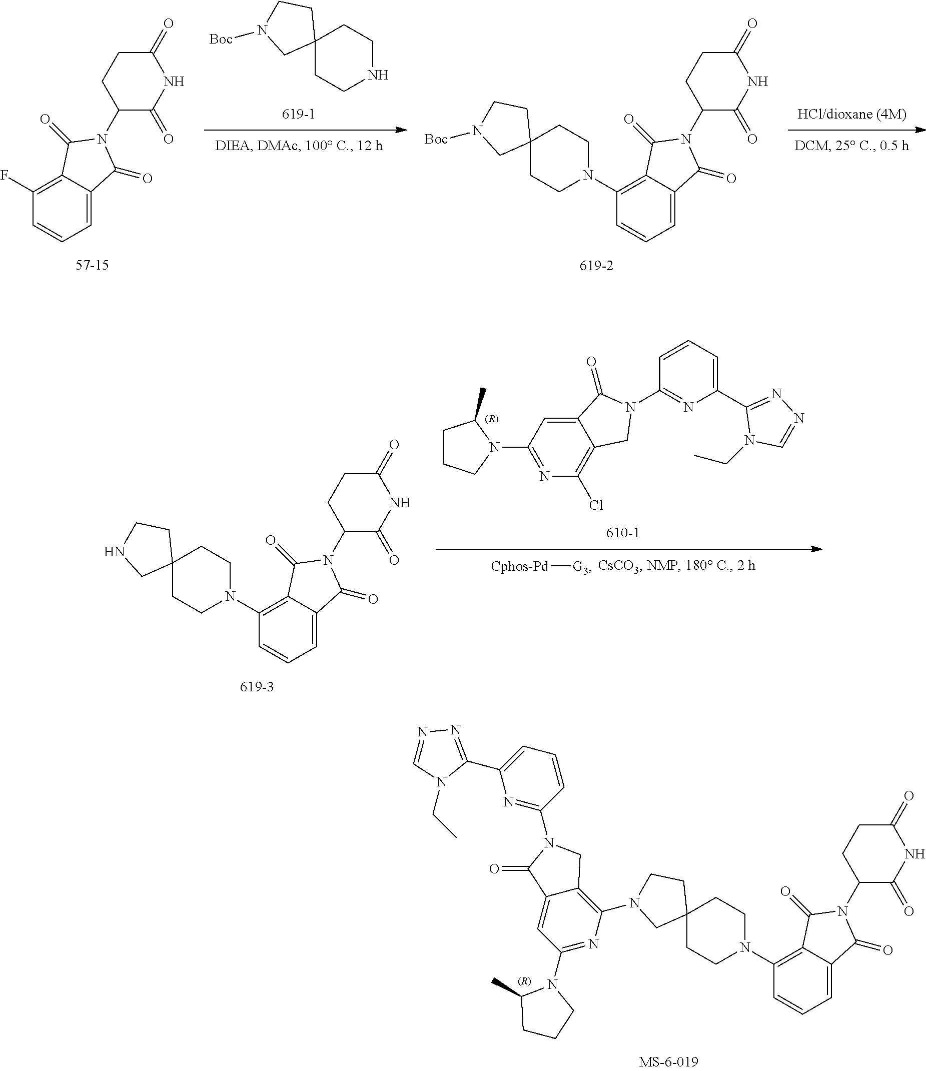

The procedure for preparing compound 610-1 is described in the synthesis of MS-6-006.

Procedure for preparation of 619-2. To a solution of compound 57-15 (5 g, 18.10 mmol, 1 eq) and compound 619-1 (4.35 g, 18.10 mmol, 1 eq) in DMAc (40 mL) was added DIEA (7.02 g, 54.30 mmol, 9.46 mL, 3 eq). The mixture was stirred at 100° C. for 12 h. The reaction mixture was poured into water (200 mL) and a suspension was formed. The suspension was filtered and the filter cake was washed with water (100 mL). The filter cake was further triturated with acetonitrile (100 mL) for 1 h and filtered. The filter cake was was washed with acetonitrile (50 mL) and dried under reduced pressure to give 619-2 (5.3 g, 10.30 mmol, 56.88% yield, 96.47% purity) as a yellow solid. LCMS: t_{R}=0.477 min, MS (ESI+) m/z=497.0 [M+1]⁺. ¹H NMR (400 MHz, CDCl₃): δ=8.04 (s, 1H), 7.64-7.53 (m, 1H), 7.39 (d, J=7.2 Hz, 1H), 7.23-7.14 (m, 1H), 4.97 (dd, J=12.0, 4.8 Hz, 1H), 3.55-3.10 (m, 8H), 2.97-2.64 (m, 3H), 2.18-2.08 (m, 1H), 1.80 (t, J=7.2 Hz, 6H), 1.48 (s, 9H).

Procedure for preparation of compound 619-3. To a solution of 619-2 (500 mg, 1.01 mmol, 1 eq) in DCM (5 mL) was added HCl/dioxane (4 M, 755.20 uL, 3 eq). The mixture was stirred at 25° C. for 0.5 h. The reaction mixture was filtered and the filter residue was concentrated under the reduced pressure to give compound 619-2 (424 mg, crude, HCl) as a yellow solid. LCMS: t_{R}=0.276 min, MS (ESI+) m/z=396.9 [M+1]⁺. ¹H NMR (400 MHz, DMSO-d₆): δ=11.08 (s, 1H), 9.65 (s, 2H), 7.69 (dd, J=8.4, 7.2 Hz, 1H), 7.34 (dd, J=8.4, 5.2 Hz, 2H), 5.09 (dd, J=12.4, 5.2 Hz, 1H), 3.33-3.17 (m, 6H), 3.02 (t, J=5.6 Hz, 2H), 2.96-2.79 (m, 1H), 2.64-2.52 (m, 2H), 2.08-1.99 (m, 1H), 1.90-1.66 (m, 6H)

Procedure for preparation of MS-6-019. A mixture of compound 619-3 (200 mg, 462.00 μmol, 1 eq, HCl) and compound 610-1 (195.84 mg, 462.00 μmol, 1 eq), CPhos Pd G3 (37.25 mg, 46.20 μmol, 0.1 eq) and Cs₂CO₃ (451.59 mg, 1.39 mmol, 3 eq) in NMP (5 mL) was degassed and purged with N2 for 3 times, and then the mixture was stirred at 180° C. for 2 h under N₂ atmosphere. The residue was purified by prep-HPLC (44%-74% ACN in water (0.05% HCl), 10 min), and lyophilized to give MS-6-019 (1.39 mg, 1.69 μmol, 3.65e-1% yield, 95.16% purity) as a yellow solid. LCMS: t_{R}=0.502 min, MS (ESI+) m/z=784.4 [M+1]⁺. ¹H NMR (400 MHz, CDCl₃): δ=8.80-8.70 (m, 1H), 8.26 (s, 1H), 8.12 (d, J=7.2 Hz, 1H), 8.02 (s, 1H), 7.96-7.88 (m, 1H), 7.61 (dd, J=8.4, 7.2 Hz, 1H), 7.42 (d, J=6.8 Hz, 1H), 7.21 (d, J=8.4 Hz, 1H), 6.17 (s, 1H), 5.23 (s, 2H), 4.98 (dd, J=12.0, 5.2 Hz, 1H), 4.65 (q, J=7.2 Hz, 2H), 4.25-4.14 (m, 1H), 3.88-3.80 (m, 2H), 3.66 (d, J=2.4 Hz, 2H), 3.48-3.32 (m, 5H), 2.94-2.88 (m, 1H), 2.86-2.79 (m, 1H), 2.79-2.72 (m, 1H), 2.23 (s, 1H), 2.11 (dd, J=11.2, 5.2 Hz, 2H), 2.04-2.01 (m, 2H), 1.98 (d, J=7.2 Hz, 2H), 1.90 (t, J=5.2 Hz, 4H), 1.75-1.71 (m, 1H), 1.26 (s, 3H), 0.89 (t, J=6.4 Hz, 3H).

### Example 48

### Synthesis of MS-6-020

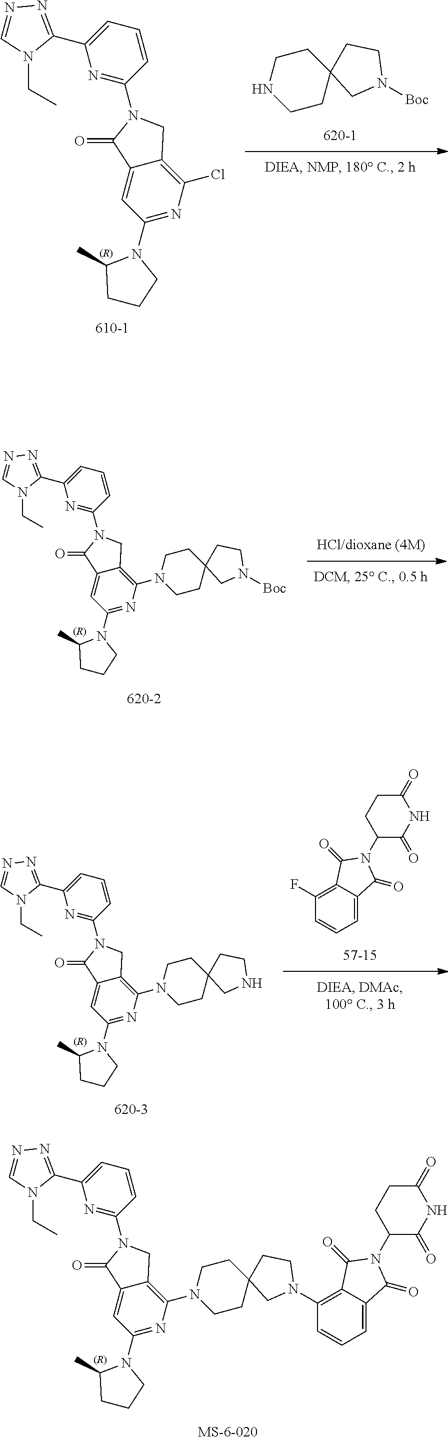

The procedure for preparing compound 610-1 is described in the synthesis of MS-6-006.

Procedure for preparation of compound 620-2. To a solution of compound 610-1 (200 mg, 471.81 μmol, 1 eq) and compound 620-1 (226.79 mg, 943.62 μmol, 2 eq) in NMP (2.5 mL) was added DIEA (182.94 mg, 1.42 mmol, 246.54 uL, 3 eq). The mixture was stirred at 180° C. for 2 h. The mixture was purified by prep-HPLC (60%-95% ACN in water (0.225% FA), 29 min) and lyophilized to give compound 3 (400 mg, 593.65 μmol, 62.91% yield, FA) as a yellow solid. LCMS: t_{R}=0.578 min, MS (ESI+) m/z=628.2[M+1]⁺. ¹H NMR (400 MHz, DMSO-d₆) δ=8.72 (s, 1H), 8.62-8.55 (m, 1H), 8.08-8.01 (m, 1H), 8.00-7.95 (m, 1H), 6.03 (s, 1H), 5.13 (s, 2H), 4.59 (q, J=6.8 Hz, 2H), 4.13 (t, J=6.0 Hz, 1H), 3.70-3.43 (m, 6H), 3.29-3.19 (m, 2H), 3.15 (s, 2H), 2.12-1.86 (m, 3H), 1.76 (t, J=6.0 Hz, 2H), 1.69-1.51 (m, 5H), 1.46 (t, J=7.2 Hz, 3H), 1.40 (s, 9H), 1.18 (d, J=6.0 Hz, 3H)

Procedure for preparation of compound 620-3. To a solution of compound 620-2 (400 mg, 593.65 μmol, 1 eq, FA) in DCM (4 mL) was added HCl/dioxane (4 M, 477.88 uL, 3.22 eq). The mixture was stirred at 25° C. for 0.5 h. The reaction mixture was filtered and the filter residue was concentrated under the reduced pressure to give a residue, and the residue was added NaHCO₃ (aq) until pH around 8, and then extracted with EtOAc (10 mL×2). The combined organic layers were dried over anhydrous Na₂SO₄, filtered and concentrated to give compound 620-3 (100 mg, 189.52 μmol, 31.92% yield) as a yellow solid. LCMS: t_{R}=0.407 min, MS (ESI+) m/z=528.3 [M+1]⁺

Procedure for preparation of MS-6-020. To a solution of compound 620-3 (100 mg, 189.52 μmol, 1 eq) and compound 57-15 (52.35 mg, 189.52 μmol, 1 eq) in DMAc (1 mL) was added DIEA (48.99 mg, 379.03 μmol, 66.02 uL, 2 eq. The mixture was stirred at 100° C. for 3 h. The residue was purified by prep-HPLC (62%-92% ACN in water (0.225% FA), 9 min) and lyophilized to give MS-6-020 (34.2 mg, 40.38 μmol, 21.31% yield, 97.98% purity, FA) as a yellow solid. LCMS: t_{R}=0.543 min, MS (ESI+) m/z=784.4 [M+1]⁺. ¹H NMR (400 MHz, DMSO-d₆): δ=11.05 (s, 1H), 8.71 (s, 1H), 8.61 (d, J=8.4 Hz, 1H), 8.29 (s, 1H), 8.11-8.02 (m, 1H), 8.02-7.94 (m, 1H), 7.64-7.53 (m, 1H), 7.14 (d, J=7.6 Hz, 2H), 6.07 (s, 1H), 5.18 (s, 2H), 5.06 (dd, J=12.8, 5.2 Hz, 1H), 4.60 (q, J=7.2 Hz, 2H), 4.15 (t, J=6.0 Hz, 1H), 3.77-3.56 (m, 6H), 3.56-3.43 (m, 3H), 3.27-3.23 (m, 1H), 2.93-2.81 (m, 1H), 2.63-2.54 (m, 2H), 2.05-1.88 (m, 6H), 1.67 (br s, 5H), 1.46 (t, J=7.2 Hz, 3H), 1.19 (d, J=6.4 Hz, 3H).

### Example 49

### Synthesis of MS-6-021

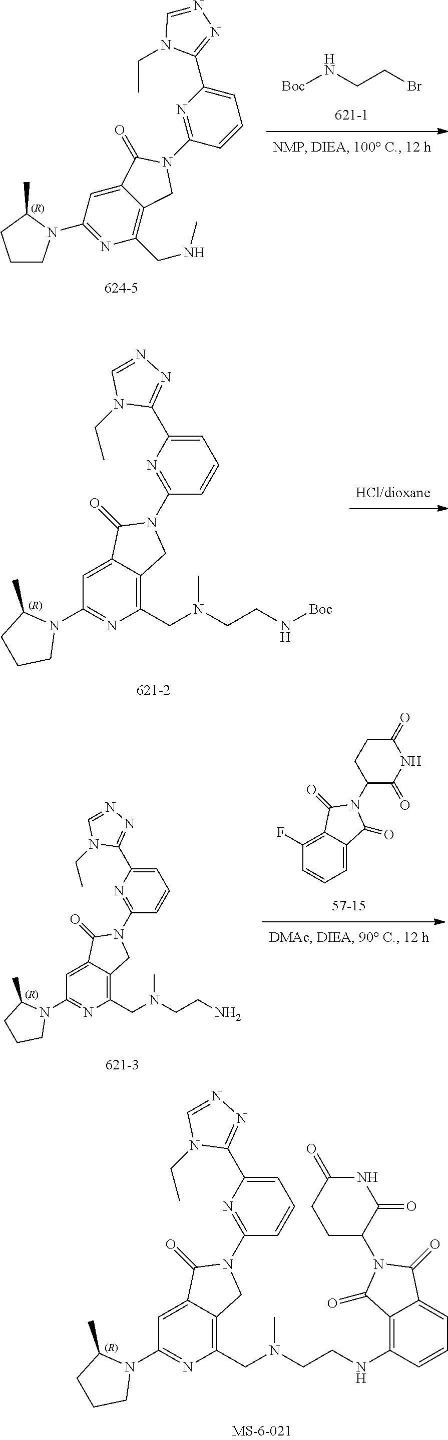

The procedure for preparing compound 624-5 is described in the synthesis of MS-6-027.

Procedure for preparation of compound 621-2. A flask charged with a stir bar was added compound 624-5 (500 mg, 1.16 mmol, 1 eq), compound 621-1 (777.17 mg, 3.47 mmol, 3 eq), DIEA (597.63 mg, 4.62 mmol, 805.43 uL, 4 eq) and NMP (2 mL). The mixture was stirred at 100° C. for 12 h. The mixture was filtered and purified by prep-HPLC (18%-48% ACN in water (0.225% FA), 10 min) to give compound 621-2 (110 mg, 191.07 μmol, 16.53% yield) as yellow solid. LCMS: t_{R}=0.406 min, MS (ESI+) m/z=576.3[M+1]⁺. ¹H NMR (400 MHz, DMSO-d₆) δ=8.73 (s, 1H), 8.61 (d, J=8.4 Hz, 1H), 8.07 (t, J=8.0 Hz, 1H), 8.00 (d, J=7.2 Hz, 1H), 6.57 (s, 1H), 5.12 (s, 2H), 4.66 (q, J=7.2 Hz, 2H), 4.25-4.13 (m, 1H), 3.68 (s, 2H), 3.53-3.49 (m, 1H), 3.36-3.24 (m, 2H), 3.10-2.96 (m, 2H), 2.45-2.37 (m, 2H), 2.27 (s, 3H), 2.09-1.91 (m, 3H), 1.69 (d, J=7.6 Hz, 1H), 1.49 (t, J=7.2 Hz, 3H), 1.27-1.12 (m, 12H).

Procedure for preparation of compound 621-3. To a solution of compound 621-2 (110 mg, 191.07 μmol, 1 eq) in DCM (1 mL) was added HCl/dioxane (4 M, 238.84 uL, 5 eq). The mixture was stirred at 25° C. for 1 h. The mixture was quenched with saturated solution of NaHCO₃ (10 mL) and extracted with DCM/MeOH (10/1, 30 mL), the combined organic phase was dried over anhydrous sodium sulfate, filtered and concentrated to give compound 621-3 (90 mg, crude) as brown oil. LCMS: t_{R}=0.417 min, MS (ESI+) m/z=476.2[M+1]⁺

Procedure for preparation of compound MS-6-021. To a solution of compound 621-3 (90 mg, 189.24 μmol, 1 eq) in NMP (1 mL) was added compound 57-15 (62.73 mg, 227.09 μmol, 1.2 eq), DIEA (73.37 mg, 567.72 μmol, 98.89 uL, 3 eq). Then the mixture was stirred at 100° C. for 12 h. The mixture was filtered and purified by prep-HPLC (10%-40% ACN in water (0.225% FA), 8 min) and prep-HPLC (42%-72% ACN in water (10 mM NH₄HCO₃), 9 min) to give MS-6-021 (7.95 mg, 10.58 μmol, 5.59% yield, 97.4% purity) as yellow solid. LCMS: t_{R}=0.378 min, MS (ESI+) m/z=732.3[M+1]⁺. ¹H NMR (400 MHz, DMSO-d₆) δ=10.95 (s, 1H), 8.64 (s, 1H), 8.56 (d, J=8.4 Hz, 1H), 8.05 (t, J=8.0 Hz, 1H), 7.95 (d, J=7.6 Hz, 1H), 7.42 (t, J=7.6 Hz, 1H), 6.92 (d, J=8.4 Hz, 1H), 6.89-6.81 (m, 1H), 6.47 (s, 1H), 6.37-6.29 (m, 1H), 5.25-5.13 (m, 2H), 4.63 (s, 1H), 4.53 (q, J=6.8 Hz, 2H), 4.16 (t, J=5.6 Hz, 1H), 3.72 (dq, J=13.2, 6.8 Hz, 2H), 3.60-3.44 (m, 1H), 3.32-3.20 (m, 3H), 2.77-2.62 (m, 3H), 2.50-2.37 (m, 5H), 2.24-2.11 (m, 1H), 2.07-1.88 (m, 3H), 1.68 (br s, 1H), 1.47-1.35 (m, 3H), 1.18 (d, J=6.0 Hz, 3H).

### Example 50

### Synthesis of MS-6-022

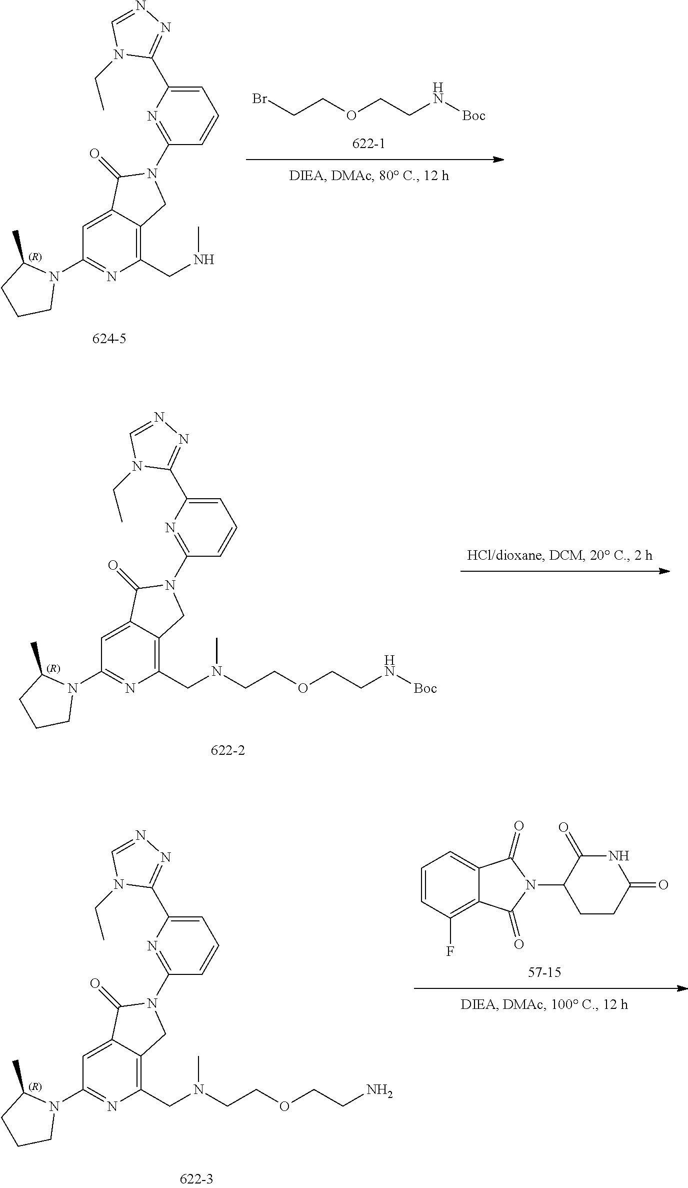

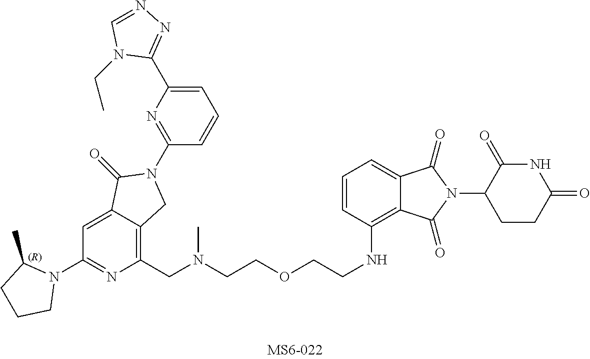

The procedure for preparing compound 624-5 is described in the synthesis of MS-6-027.

Procedure for preparation of compound 622-2. To a solution of compound 624-5 (500 mg, 1.07 mmol, 1 eq, HCl) in DMAC (5 mL) was added DIEA (688.96 mg, 5.33 mmol, 928.51 uL, 5 eq) and compound 622-1 (285.88 mg, 1.07 mmol, 1 eq), the resulting mixture was stirred at 90° C. for 12 h. The reaction was filtered and purified by Prep-HPLC (0%-50% ACN in water (0.225 FA), 10 min) to give compound 622-2 (120 mg, 164.58 μmol, 15.44% yield, 85% purity) as a brown solid. LCMS: t_{R}=0.409 min, MS (ESI+) m/z=620.4 [M+1]⁺

Procedure for preparation of compound 622-3. To a solution of compound 622-2 (100 mg, 161.35 μmol, 1 eq) in DCM (1 mL) was added HCl/dioxane (4 M, 0.5 mL, 12.40 eq) and the mixture was stirred at 25° C. for 0.5 h. The reaction mixture was concentrated under reduced pressure to give compound 622-3 (80 mg, crude, HCl) as a yellow solid, used directly. LCMS: t_{R}=0.320 min, MS (ESI+) m/z=520.4[M+1]⁺

Procedure for preparation of compound MS-6-022. A mixture of compound 622-3 (80 mg, 143.86 μmol, 1 eq, HCl), compound 57-15 (39.74 mg, 143.86 μmol, 1 eq) and DIEA (55.78 mg, 431.58 μmol, 75.17 uL, 3 eq) in DMAC (1 mL) was stirred at 100° C. for 12 h. The reaction mixture was filtered and purified by prep-HPLC (15%-45% ACN in water (0.225% FA), 8 min) to give MS-6-022 (5.2 mg, 6.22 μmol, 4.32% yield, 98.26% purity, FA) as a yellow solid. LCMS: t_{R}=1.545 min, MS (ESI+) m/z=776.3[M+1]⁺. ¹H NMR (400 MHz, DMSO-d₆): δ=11.22-10.92 (m, 1H), 8.70 (s, 1H), 8.53-8.43 (m, 1H), 8.37 (s, 1H), 7.99-7.86 (m, 2H), 7.41 (t, J=8.0 Hz, 1H), 6.95 (d, J=7.2 Hz, 1H), 6.82 (d, J=9.2 Hz, 1H), 6.57 (s, 1H), 6.39 (d, J=2.8 Hz, 1H), 5.19 (s, 2H), 5.05-4.90 (m, 1H), 4.65 (q, J=7.2 Hz, 2H), 4.31-4.10 (m, 1H), 3.74 (s, 2H), 3.63-3.49 (m, 6H), 3.24 (d, J=2.8 Hz, 2H), 2.92-2.82 (m, 1H), 2.64-2.55 (m, 4H), 2.30 (s, 3H), 2.10-1.95 (m, 4H), 1.73-1.65 (m, 1H), 1.48 (t, J=7.2 Hz, 3H), 1.20 (d, J=6.4 Hz, 3H).

### Example 51

### Synthesis of MS-6-023

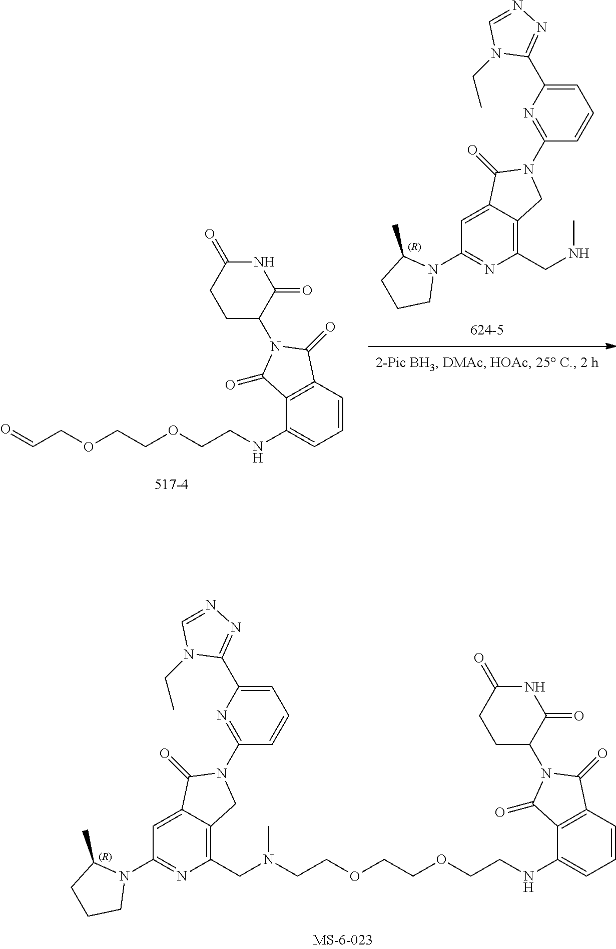

The procedure for preparing compound 517-4 described in the synthesis of MS-5-017.

The procedure for preparing compound 624-5 described in the synthesis of MS-6-027.

Procedure for preparation of MS-6-023. To a solution of compound 517-4 (100 mg, 247.90 μmol, 1 eq) and compound 624-5 (116.26 mg, 247.90 μmol, 1 eq, HCl) in DMAc (1 mL) and HOAc (0.1 mL) was added 2-Pic BH₃ (31.82 mg, 297.48 μmol, 1.2 eq) and the mixture was stirred at 25° C. for 2 h. The reaction mixture was concentrated under the reduced pressure to give a residue. The residue was purified by prep-HPLC (33%-63% ACN in water (10 mM NH₄HCO₃), 8 min) and prep-HPLC (16%-46% ACN in water (0.225% FA), 10 min) and lyophilized to give MS-6-023 (9.25 mg, 10.50 μmol, 4.24% yield, 98.33% purity, FA) as a yellow solid. LCMS: t_{R}=0.677 min, MS (ESI+) m/z=820.4 [M+1]⁺. ¹H NMR (400 MHz, DMSO-d₆) δ=11.08 (s, 1H), 8.73 (s, 1H), 8.61 (d, J=8.4 Hz, 1H), 8.43-8.33 (m, 1H), 8.12-8.03 (m, 1H), 8.02-7.97 (m, 1H), 7.54-7.45 (m, 1H), 6.99 (d, J=7.6 Hz, 2H), 6.57 (s, 1H), 6.48 (t, J=5.6 Hz, 1H), 5.18 (s, 2H), 5.01 (dd, J=12.8, 5.2 Hz, 1H), 4.65 (q, J=7.2 Hz, 2H), 4.18 (t, J=6.0 Hz, 1H), 3.71 (s, 2H), 3.57-3.47 (m, 4H), 3.40 (d, J=5.6 Hz, 6H), 3.28 (d, J=6.0 Hz, 2H), 2.91-2.81 (m, 1H), 2.60-2.53 (m, 4H), 2.27 (s, 3H), 2.07-1.92 (m, 4H), 1.75-1.62 (m, 1H), 1.48 (t, J=7.2 Hz, 3H), 1.18 (d, J=6.4 Hz, 3H).

### Example 52

### Synthesis of MS-6-024

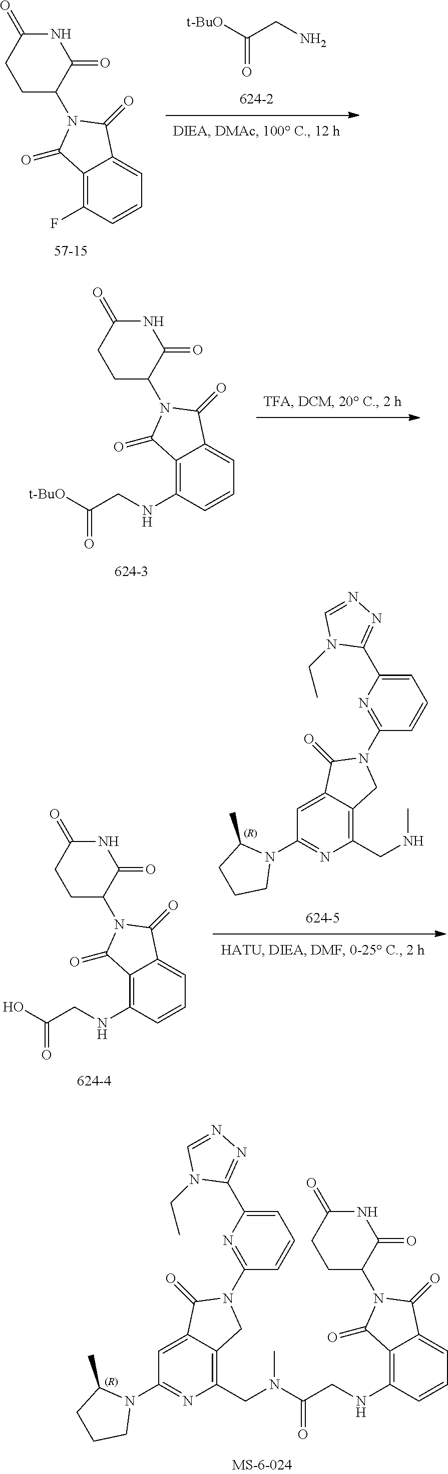

The procedure for preparing compound 624-5 described in the synthesis of MS-6-027.

Procedure for preparation of compound 624-3. To a solution of compound 57-15 (1 g, 3.62 mmol, 1 eq) and compound 624-2 (712.33 mg, 5.43 mmol, 1.5 eq) in DMAc (5 mL) was added DIEA (1.40 g, 10.86 mmol, 1.89 mL, 3 eq), the reaction mixture was stirred at 100° C. for 12 h. The reaction mixture was quenched by addition H₂O (10 mL) and extracted with EtOAc (60×3 mL).

The combined organic layers was washed with brine (80 mL) and concentrated under reduced pressure to get a residue. The residue was purified by flash silica gel chromatography (SiO2, PE/EA 100/1=0/1) to give compound 624-3 (655 mg, 1.69 mmol, 46.70% yield) as a yellow oil. LCMS: t_{R}=0.436 min, MS (ESI+) m/z=331.8[M-56]⁺. ¹H NMR (400 MHz, DMSO-d₆): δ=11.11 (s, 1H), 7.59 (dd, J=8.4, 7.2 Hz, 1H), 7.11-7.07 (m, 1H), 7.01-6.96 (m, 1H), 6.85 (t, J=6.0 Hz, 1H), 5.08 (dd, J=12.8, 5.4 Hz, 1H), 4.10 (d, J=6.0 Hz, 2H), 2.96-2.84 (m, 1H), 2.65-2.54 (m, 2H), 2.09-2.01 (m, 1H), 1.44 (s, 9H)

Procedure for preparation of compound 624-4. To a solution of compound 624-3 (300 mg, 774.42 μmol, 1 eq) in DCM (2 mL) was added TFA (770.00 mg, 6.75 mmol, 0.5 mL, 8.72 eq), the reaction mixture was stirred at 20° C. for 12 h. The reaction mixture was quenched by addition ACN (2 mL) and filtered to give compound 624-4 (195 mg, crude) as a yellow solid. LCMS: t_{R}=0.298 min, MS (ESI+) m/z=331.9[M+1]⁺

Procedure for preparation of compound MS-6-024. To a solution of compound 4 (70 mg, 211.30 μmol, 1.2 eq) in DMF (2 mL) was added HATU (100.43 mg, 264.13 μmol, 1.5 eq) and stirred at 0° C. for 30 min, then the mixture was added compound 624-5 (76.16 mg, 176.08 μmol, 1 eq), DIEA (68.27 mg, 528.25 μmol, 92.01 uL, 3 eq) and stirred at 25° C. for 1.5 h. The reaction mixture was filtered and purified by prep-HPLC (41%-71% ACN in water (0.225% FA), 10 min) to give MS-6-024 (34.44 mg, 46.18 μmol, 26.23% yield, 100% purity) as a yellow solid. LCMS: t_{R}=0.469 min, MS (ESI+) m/z=746.3[M+1]⁺. ¹H NMR (400 MHz, DMSO-d₆): δ=11.11 (s, 1H), 8.66-8.61 (m, 1H), 8.15-8.06 (m, 1H), 8.04-7.98 (m, 1H), 7.57-7.50 (m, 1H), 7.13-6.98 (m, 3H), 6.66-6.61 (m, 1H), 5.17-5.03 (m, 3H), 4.81-4.59 (m, 4H), 4.42-4.02 (m, 2H), 3.59-3.44 (m, 1H), 3.26-3.11 (m, 1H), 2.97-2.85 (m, 2H), 2.64-2.58 (m, 2H), 2.10-1.89 (m, 5H), 1.73-1.60 (m, 1H), 1.51-1.41 (m, 3H), 1.18 (−t, J=6.4 Hz, 2H), 1.06 (d, J=6.4 Hz, 1H).

### Example 53

### Synthetic Route for MS-6-025

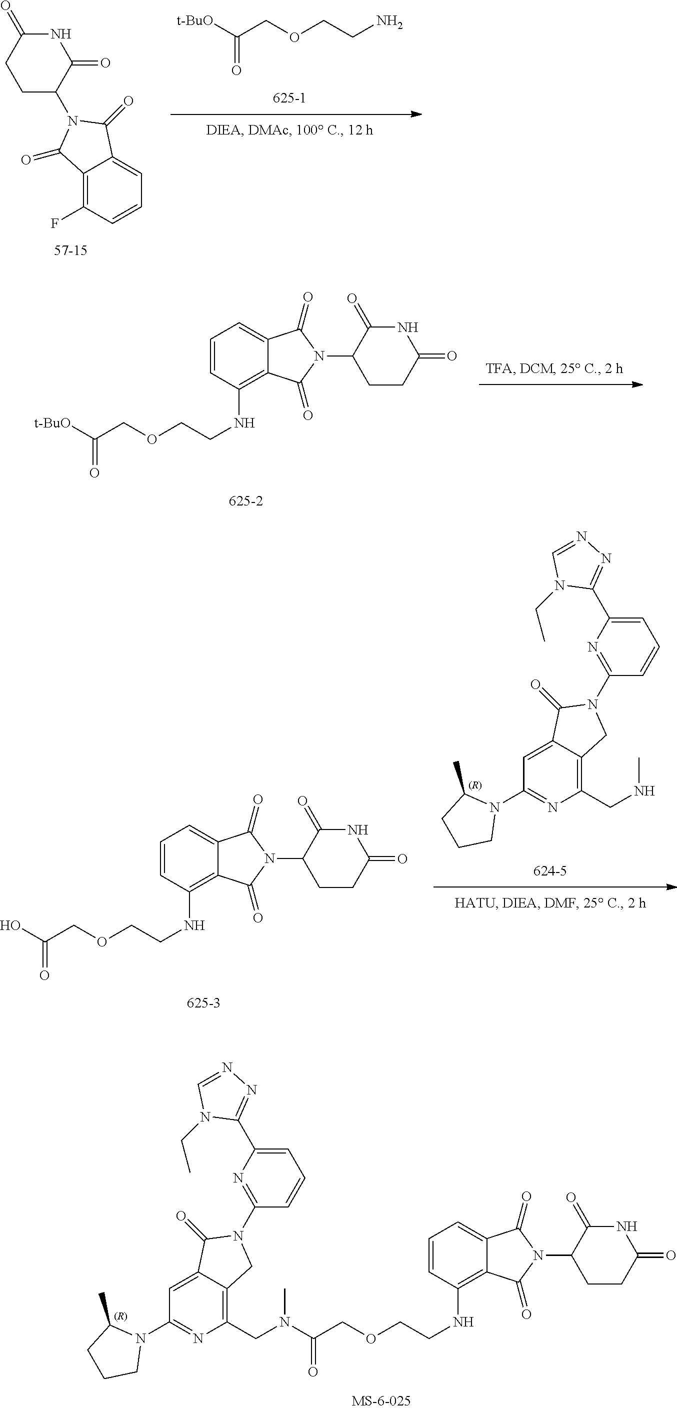

The procedure for preparing compound 624-5 is described in the synthesis of MS-6-027.

Procedure for preparation of compound 625-2. To a solution of compound 57-15 (1 g, 3.62 mmol, 1 eq) and compound 625-1 (951.55 mg, 5.43 mmol, 1.50 eq) in DMAc (5 mL) was added DIEA (1.40 g, 10.86 mmol, 1.89 mL, 3 eq), the reaction mixture was stirred at 100° C. for 12 h. The reaction mixture was quenched by addition H₂O (10 mL) and extracted with EtOAc (60×3 mL), the combined organic layers was washed brine (100 mL) and concentrated under reduced pressure to give a residue. The residue was purified by prep-HPLC (35%-66% ACN in water (0.225% FA), 8 min) and lyophilized to give compound 625-2 (335 mg, 776.47 μmol, 21.45% yield) as a yellow solid. LCMS: t_{R}=0.438 min, MS (ESI+) m/z=432.1 [M+1]⁺. ¹H NMR (400 MHz, DMSO-d₆) δ=11.11 (s, 1H), 7.63-7.56 (m, 1H), 7.16 (d, J=8.4 Hz, 1H), 7.05 (d, J=7.2 Hz, 1H), 6.66 (t, J=5.4 Hz, 1H), 5.06 (dd, J=12.8, 5.2 Hz, 1H), 4.04 (s, 2H), 3.66 (t, J=5.2 Hz, 2H), 3.50 (q, J=5.6 Hz, 2H), 2.91-2.83 (m, 1H), 2.71-2.59 (m, 2H), 2.08-1.99 (m, 1H), 1.41 (s, 9H).

Procedure for preparation of compound 625-3. To a solution of compound 625-2 (200 mg, 463.57 μmol, 1 eq) in DCM (1 mL) was added TFA (770.00 mg, 6.75 mmol, 0.5 mL, 14.57 eq) and the mixture was stirred at 25° C. for 2 h. The reaction mixture was concentrated under reduced pressure to give compound 625-3 (80 mg, 213.14 μmol, 45.98% yield) as a green solid. LCMS: t_{R}=0.319 min, MS (ESI+) m/z=375.8 [M+1]⁺

Procedure for preparation of MS-6-025. A mixture of compound 625-3 80 mg, 213.14 μmol, 1 eq) in DMF (1 mL) was added compound 624-5 (99.96 mg, 213.14 μmol, 1 eq, HCl), HATU (121.57 mg, 319.72 μmol, 1.5 eq) and DIEA (82.64 mg, 639.43 μmol, 111.38 uL, 3 eq) and the mixture was stirred at 25° C. for 2 h. The residue was purified by prep-HPLC (35%-65% ACN in water (0.225% FA), 10 min) and lyophilized to give MS-6-025 (19.39 mg, 23.20 μmol, 10.88% yield, FA) as a yellow solid. LCMS: t_{R}=0.438 min, MS (ESI+) m/z=790.1 [M+1]⁺. ¹H NMR (400 MHz, DMSO-d₆) δ=11.07 (s, 1H), 8.73 (d, J=14.4 Hz, 1H), 8.60 (dd, J=8.0, 6.0 Hz, 1H), 8.14 (s, 1H), 8.08 (q, J=8.0 Hz, 1H), 8.03-7.97 (m, 1H), 7.52 (q, J=7.6 Hz, 1H), 7.08 (d, J=8.4 Hz, 1H), 7.00 (d, J=7.2 Hz, 1H), 6.72-6.52 (m, 2H), 5.18-4.91 (m, 3H), 4.75-4.50 (m, 4H), 4.47-4.24 (m, 2H), 4.23-4.12 (m, 1H), 3.67 (d, J=5.2 Hz, 2H), 3.60-3.38 (m, 4H), 3.05 (s, 2H), 2.92-2.81 (m, 2H), 2.64-2.55 (m, 2H), 2.11-1.89 (m, 4H), 1.72-1.62 (m, 1H), 1.47 (td, J=9.6, 7.2 Hz, 3H), 1.16 (dd, J=19.0, 6.0 Hz, 3H).

### Example 54

### Synthetic Route for MS-6-026

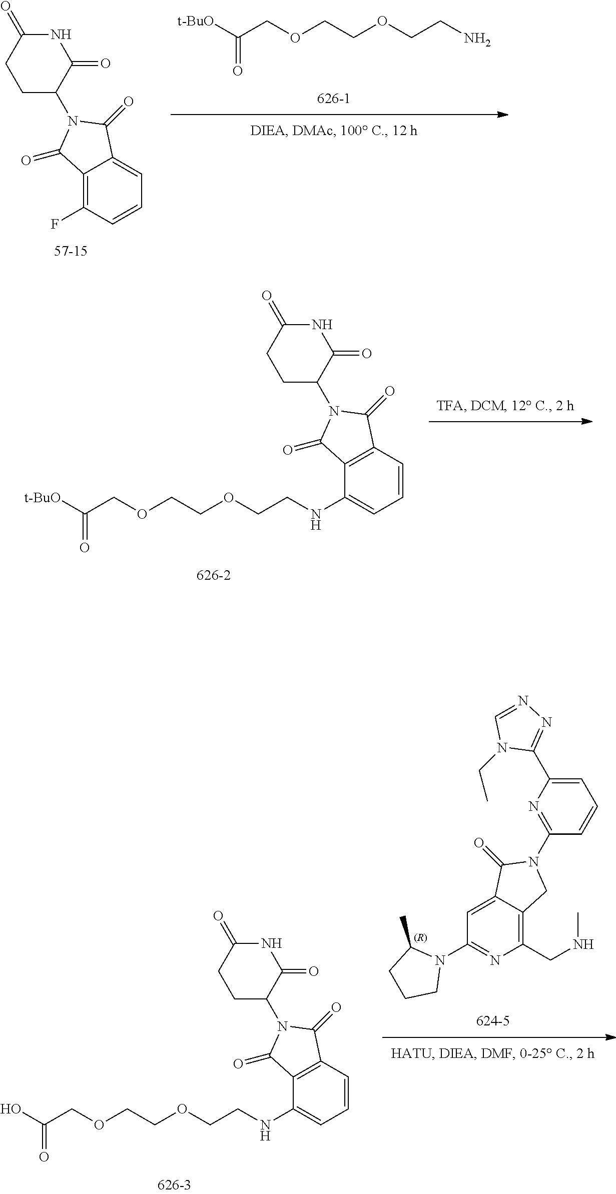

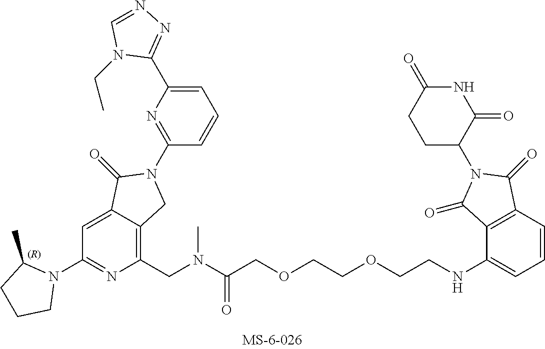

The procedure for preparing compound 624-5 is described in the synthesis of MS-6-027.

Procedure for preparation of 624-2. To a solution of compound 57-15 (1 g, 3.62 mmol, 1 eq) and compound 626-1 (1.19 g, 5.43 mmol, 1.5 eq) in DMAc (5 mL) was added DIEA (1.40 g, 10.86 mmol, 1.89 mL, 3 eq). The reaction mixture was stirred at 100° C. for 12 h. The reaction mixture was quenched by addition H₂O (10 mL) and extracted with EtOAc (60×3 mL), the combined organic layers were washed with brine (90 mL) and concentrated under reduced pressure to give a residue. The residue was purified by flash silica gel chromatography (SiO2, PE/EtOAc 100/1=0/1) to give compound 624-2 (891 mg, 1.87 mmol, 51.76% yield) as a green oil. ¹H NMR (400 MHz, DMSO-d₆): δ=11.10 (s, 1H), 7.59 (dd, J=8.4, 7.2 Hz, 1H), 7.15 (d, J=8.4 Hz, 1H), 7.04 (d, J=6.8 Hz, 1H), 6.61 (s, 1H), 5.06 (dd, J=12.8, 5.4 Hz, 1H), 3.98 (s, 2H), 3.65-3.61 (m, 2H), 3.51-3.42 (m, 6H), 3.17 (s, 1H), 2.91-2.82 (m, 1H), 2.66-2.53 (m, 2H), 2.07-2.01 (m, 1H), 1.41 (s, 9H).

Procedure for preparation of compound 624-3. To a solution of compound 624-2 (200 mg, 420.62 mol, 1 eq) in DCM (2 mL) was added TFA (770.00 mg, 6.75 mmol, 0.5 mL, 16.05 eq), the reaction mixture was stirred at 20° C. for 12 h. The reaction mixture was concentrated under reduced pressure to give compound 624-3 (100 mg, crude) as a brown solid. LCMS: t_{R}=0.334 min, MS (ESI+) m/z=420.0 [M+1]⁺

Procedure for preparation of compound MS-6-026. To a solution of compound 624-3 (80 mg, 190.76 μmol, 1 eq) in DMF (1 mL) was added HATU (108.80 mg, 286.13 μmol, 1.5 eq) under 0° C. and stirred at 0° C. for 30 min, then compound 624-5 (89.46 mg, 190.76 μmol, 1 eq, HCl) and DIEA (73.96 mg, 572.27 μmol, 99.68 μL, 3 eq) was added to the reaction mixture, the resulting mixture was warmed to 25° C. and stirred at 25° C. for another 1.5 h. The reaction mixture was filtered. The residue was purified by prep-HPLC (35%-65% ACN in water (0.225% FA), 10 min) and neutralized with NaHCO₃ aqueous solution to give compound MS-6-026 (32.47 mg, 38.10 mol, 19.98% yield, 97.86% purity) as a yellow solid. LCMS: t_{R}=0.636 min, MS (ESI+) m/z=834.3 [M+1]⁺. ¹H NMR (400 MHz, DMSO-d₆): δ=11.09 (s, 1H), 8.77 (s, 1H), 8.60 (dd, J=8.4, 2.8 Hz, 1H), 8.12-8.05 (m, 1H), 8.04-7.96 (m, 1H), 7.59-7.46 (m, 1H), 7.14-6.92 (m, 2H), 6.66-6.46 (m, 2H), 5.18-4.91 (m, 3H), 4.77-4.48 (m, 4H), 4.40-4.08 (m, 3H), 3.72-3.45 (m, 8H), 3.41 (d, J=4.4 Hz, 2H), 3.04 (s, 2H), 2.92-2.83 (m, 2H), 2.62-2.53 (m, 2H), 2.14-1.87 (m, 4H), 1.69 (s, 1H), 1.57-1.40 (m, 3H), 1.16 (dd, J=18.2, 6.4 Hz, 3H).

### Example 55

### Synthesis of MS-6-027

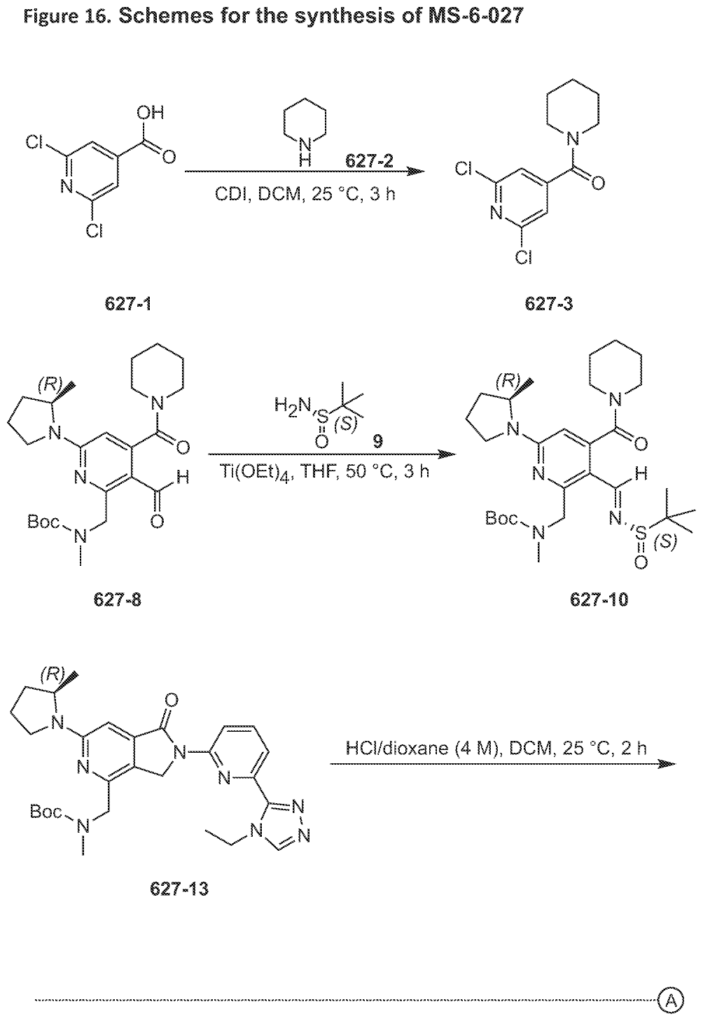

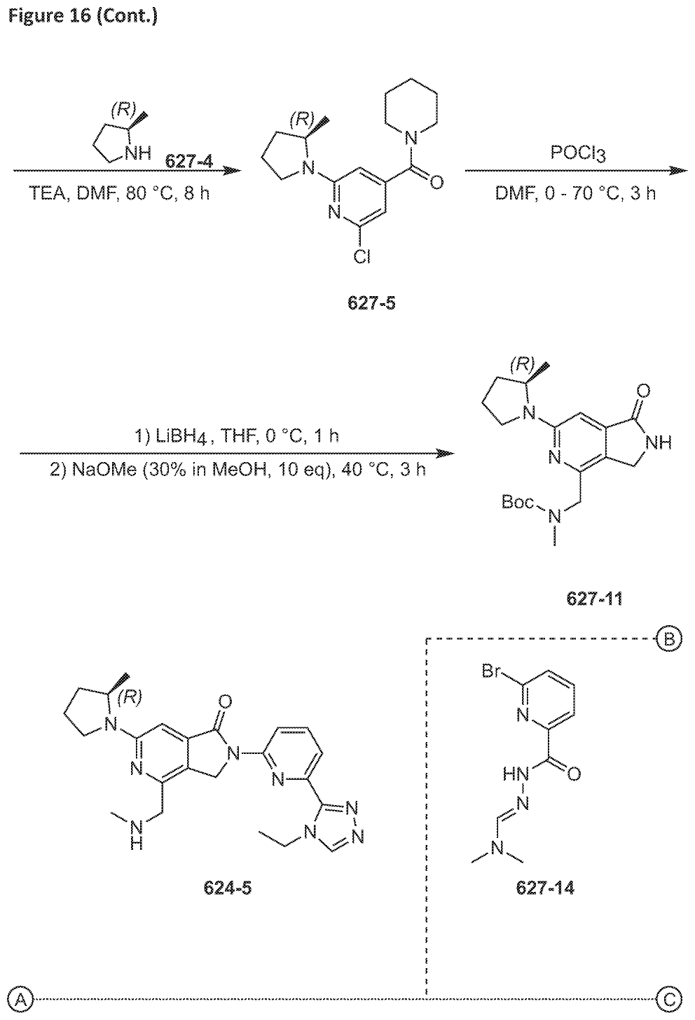

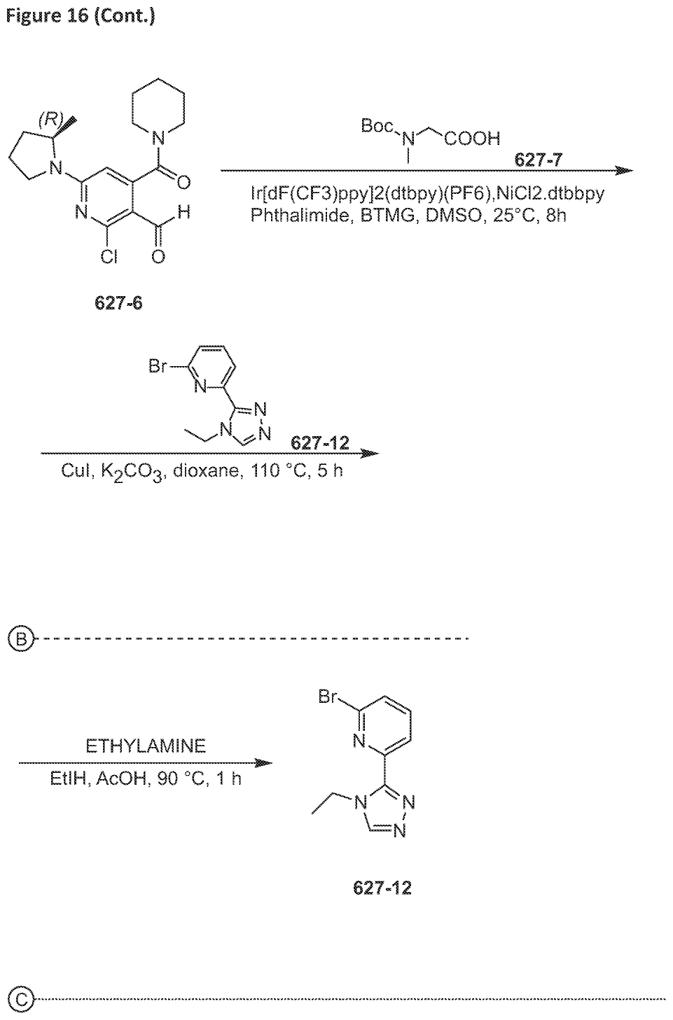

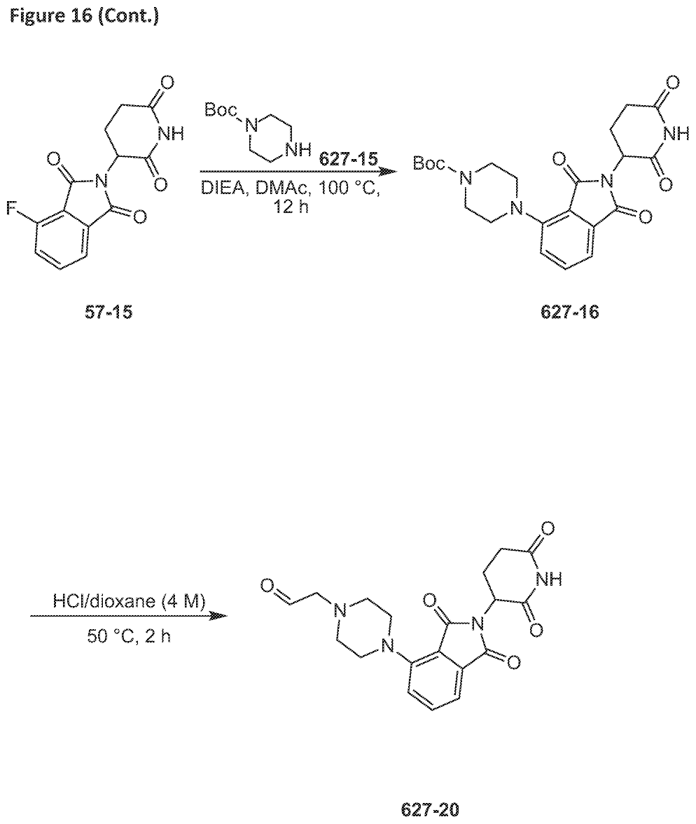

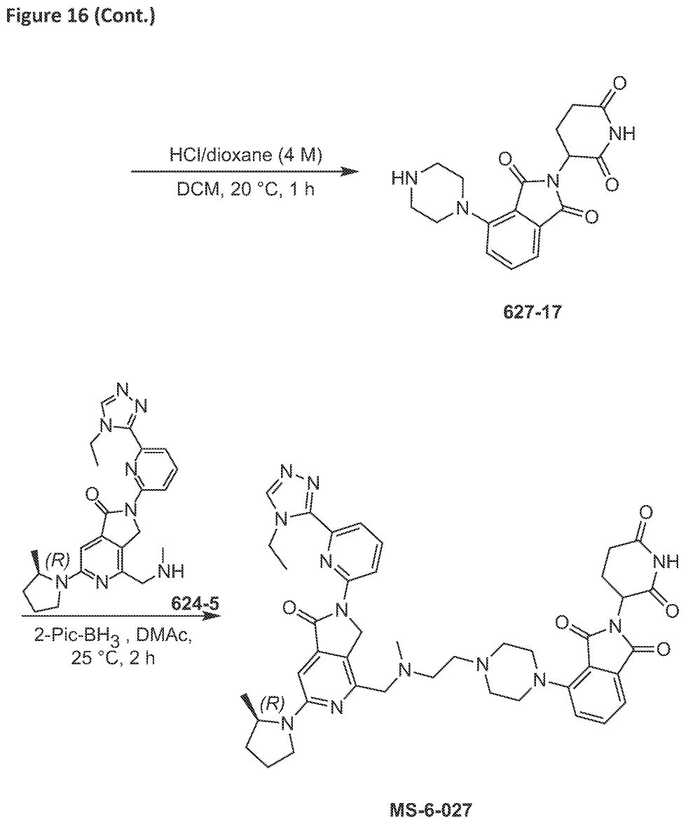

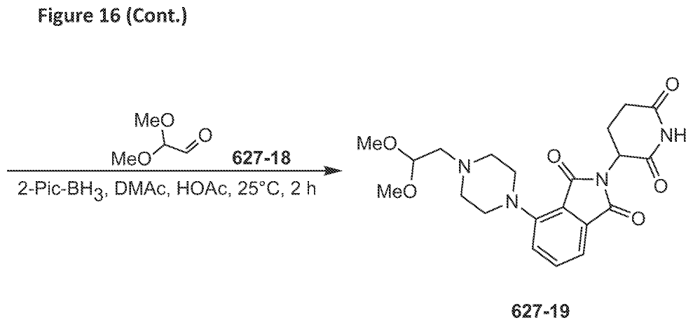

The reaction scheme for the synthesis of MS-6-027 is shown in FIG. 16.

Procedure for preparation of compound 627-3. A mixture of compound 627-1 (90 g, 468.75 mmol, 1 eq), compound 627-2 (43.90 g, 515.63 mmol, 50.92 mL, 1.1 eq), CDI (152.02 g, 937.50 mmol, 2.0 eq) in DCM (300 mL) was degassed and purged with N₂ for 3 times, and then the mixture was stirred at 25° C. for 3 h under N₂ atmosphere. The mixture was concentrated in vacuum to give a residue. The residue was triturated with PE/EA=1/1 at 25° C. for 30 min to give compound 627-3 (110 g, 424.50 mmol, 90.56% yield) as a white solid. LCMS: t_{R}=0.422 min, MS (ESI+) m/z=258.8 [M+1]⁺. ¹H NMR (400 MHz, CDCl₃) δ=7.24-7.22 (m, 2H), 3.68 (t, J=5.2 Hz, 2H), 3.32-3.25 (m, 2H), 1.71-1.67 (m, 4H), 1.56 (d, J=4.4 Hz, 2H).

Procedure for preparation of compound 627-5. A mixture of compound 627-3 (97 g, 374.33 mmol, 1 eq), compound 627-4 (91.04 g, 748.66 mmol, 2.0 eq, HCl), Et₃N (151.51 g, 1.50 mol, 208.41 mL, 4.0 eq) in DMF (400 mL) was degassed and purged with N₂ for 3 times, and then the mixture was stirred at 80° C. for 8 h under N₂ atmosphere. The reaction mixture was poured into water (300 mL) and extracted with ethyl acetate (300 mL×3). The combined organic phase was dried by over Na₂SO₄, filtered and concentrated in vacuo to afford crude. The residue was purified by column chromatography (SiO₂, Petroleum ether/Ethyl acetate=3/1 to 1/1) to give compound 627-5 (82 g, 266.39 mmol, 71.17% yield) as a yellow oil. LCMS: t_{R}=0.506 min, MS (ESI+) m/z=309.5 [M+1]⁺. ¹H NMR (400 MHz, CDCl₃) δ=6.43 (d, J=0.8 Hz, 1H), 6.18 (s, 1H), 4.21-4.13 (m, 1H), 3.76-3.63 (m, 2H), 3.57-3.50 (m, 1H), 3.33 (d, J=5.6 Hz, 2H), 3.26-3.14 (m, 1H), 2.16-1.94 (m, 4H), 1.75-1.62 (m, 6H), 1.21 (d, J=6.4 Hz, 3H).

Procedure for preparation of compound 627-6. To a solution of POCl₃ (49.81 g, 324.87 mmol, 30.19 mL, 5.0 eq) in DMF (100 mL) at 0° C. After addition, the mixture was stirred for 0.5 h, and then compound 627-5 (20 g, 64.97 mmol, 1 eq) was added dropwise. The resulting mixture was stirred at 70° C. for 2.5 h. The reaction mixture was poured into ice-water (100 mL), adjust to pH=7 with NaHCO₃ aqueous solution and extracted with ethyl acetate (300 mL×3). The combined organic phase was dried by over Na₂SO₄, filtered and concentrated in vacuo to afford crude. The residue was triturated with EA (150 mL) to give compound 627-6 (35 g, 104.22 mmol, 53.47% yield) as a brown solid. LCMS: t_{R}=0.462 min, MS (ESI+) m/z=336.2 [M+1]⁺. ¹H NMR (400 MHz, CDCl₃): δ=10.19 (s, 1H), 6.09 (s, 1H), 4.64-3.93 (m, 1H), 3.88-3.62 (m, 3H), 3.56-3.28 (m, 1H), 3.18-3.05 (m, 2H), 2.18-2.01 (m, 3H), 1.88-1.77 (m, 2H), 1.70-1.57 (m, 4H), 1.44-1.35 (m, 1H), 1.25 (s, 3H).

Procedure for preparation of compound 627-8. A mixture of compound 627-6 (10.00 g, 29.78 mmol, 1 eq), compound 627-7 (8.45 g, 44.67 mmol, 1.5 eq), Ir[dF(CF₃)ppy]₂(dtbpy)(PF₆) (334.10 mg, 297.80 μmol, 0.01 eq), NiCl₂.dtbbpy (11.85 g, 29.78 mmol, 1.00 eq), Phthalimide (4.38 g, 29.78 mmol, 1 eq), 2-tert-butyl-1,1,3,3-tetramethyl-guanidine (7.65 g, 44.67 mmol, 8.97 mL, 1.5 eq) in DMSO (100 mL) was degassed and purged with N₂ for 3 times, and then the mixture was stirred at 25° C. for 8 h under N₂ atmosphere. The mixture was filtered and concentrated in vacuum to give a residue. The residue was purified by column chromatography (SiO₂, Petroleum ether/Ethyl acetate=3/1 to 1/1) to give compound 627-8 (8.0 g, 18.00 mmol, 60.43% yield) as a yellow solid. LCMS: t_{R}=0.506 min, MS (ESI+) m/z=309.5 [M+1]⁺. ¹H NMR (400 MHz, CDCl₃) δ=9.97 (s, 1H), 6.07 (s, 1H), 4.79-4.55 (m, 2H), 3.80-3.43 (m, 4H), 3.20 (d, J=4.4 Hz, 2H), 3.00 (s, 2H), 2.96-2.86 (m, 2H), 2.05 (s, 4H), 1.68 (s, 6H), 1.47-1.29 (m, 9H), 1.24 (d, J=8.8 Hz, 3H).

Procedure for preparation of compound 627-10. A mixture of compound 627-8 (12 g, 26.99 mmol, 1 eq) in THE (70 mL) was added Ti(i-PrO)₄ (19.18 g, 67.48 mmol, 19.92 mL, 2.5 eq), then compound 627-9 (3.93 g, 32.39 mmol, 1.2 eq) was added to the reaction mixture and the resulting mixture was stirred at 50° C. for 3 h. The reaction mixture was quenched by aq NaHCO₃ (100 mL) and DCM (200 mL), then filtered and the layer was extracted with DCM (200 mL), the organic layer was concentrated under reduced pressure to give a residue. The residue was purified by flash silica gel chromatography (SiO₂, Petroleum ether/Ethyl acetate=100/1 to 2/1) to give compound 627-10 (82 g, 266.39 mmol, 71.17% yield) as a yellow solid. LCMS: t_{R}=0.550 min, MS (ESI+) m/z=548.2 [M+1]⁺. ¹H NMR (400 MHz, DMSO-d₆) δ=8.42 (s, 1H), 6.24 (s, 1H), 4.86-4.51 (m, 4H), 3.56 (s, 4H), 3.23-3.02 (m, 4H), 2.96 (d, J=12.8 Hz, 5H), 1.71 (d, J=8.8 Hz, 2H), 1.41 (s, 9H), 1.21 (s, 2H), 1.13 (s, 9H).

Procedure for preparation of compound 627-11. To a solution of compound 627-10 (10 g, 18.26 mmol, 1 eq) in THE (80 mL) was added LiBH₄ (477.23 mg, 21.91 mmol, 1.2 eq) at 0° C. and stirred at 0° C. for 1 h, then NaOMe (5 M, 7.30 mL, 30% purity, 2 eq) was slowly added to the reaction mixture and the resulting mixture was heated to 40° C. and stirred at 40° C. for 3 h. The mixture was quenched by aq NH₄Cl (300 mL) and extracted with EtOAc (300 mL×3), the combined organic layers were concentrated under reduced pressure to give a residue. The reaction mixture was purified by flash silica gel chromatography (SiO₂, Petroleum ether/Ethyl acetate=100/1 to 0/1) to give compound 627-11 (4.3 g, 11.93 mmol, 65.34% yield) as a yellow solid. LCMS: t_{R}=0.359 min, MS (ESI+) m/z=361.0 [M+1]⁺. ¹H NMR (400 MHz, DMSO-d₆) δ=8.66 (s, 1H), 6.47 (s, 1H), 4.40 (d, J=5.2 Hz, 2H), 4.23 (s, 2H), 4.20-4.14 (m, 1H), 3.53-3.45 (m, 1H), 3.30-3.22 (m, 1H), 2.88 (s, 3H), 2.05-1.92 (m, 3H), 1.72-1.63 (m, 1H), 1.44-1.27 (m, 9H), 1.19-1.14 (m, 3H).

Procedure for preparation of compound 627-12. To a solution of compound 627-14 (80 g, 295.08 mmol, 1 eq) in EtOH (300 mL) and AcOH (100 mL) was added ETHYLAMINE (4 M, 221.31 mL, 3 eq) and the mixture was stirred at 90° C. for 1 h. The reaction mixture was quenched with aq. NaHCO₃ (200 mL) and extracted with EtOAc (300 mL×3), the combined organic layers were concentrated under reduced pressure to give a residue to give compound 627-12 (80 g, crude) as a yellow solid. LCMS: t_{R}=0.353 min, MS (ESI+) m/z=252.7 [M+1]⁺. ¹H NMR (400 MHz, DMSO-d₆) δ=8.75 (s, 1H), 8.19 (dd, J=7.6, 0.4 Hz, 1H), 7.95 (t, J=7.6 Hz, 1H), 7.78 (dd, J=8.0, 0.8 Hz, 1H), 4.47 (q, J=7.2 Hz, 2H), 1.38 (t, J=7.2 Hz, 3H).

Procedure for preparation of compound 627-13. A mixture of compound 627-12 (3 g, 11.85 mmol, 1 eq), compound 627-11 (4.27 g, 11.85 mmol, 1 eq), CuI (564.36 mg, 2.96 mmol, 0.25 eq) and K₂CO₃ (3.28 g, 23.71 mmol, 2 eq) in dioxane (70 mL) was degassed and purged with N₂ for 3 times and the mixture was stirred at 110° C. for 5 h. The reaction mixture was quenched with H₂O (200 ml) and filtered, the cake was dried in vacuum to give a residue. The residue was triturated with MeCN (30 mL) at 25° C. for 30 min to give compound 627-13 (3.7 g, 6.95 mmol, 58.61% yield) as a brown solid. LCMS: t_{R}=0.495 min, MS (ESI+) m/z=533.2 [M+1]⁺

Procedure for preparation of compound 624-5. To a solution of compound 627-13 (3.7 g, 6.95 mmol, 1 eq) in DCM (20 mL) was added HCl/dioxane (4 M, 10 mL, 5.76 eq) and the mixture was stirred at 25° C. for 2 h. The reaction mixture was filtered. The residue was purified by prep-HPLC (10%-40% ACN in water (0.225% FA), 15 min) and lyophilized to give compound 624-5 (2.8 g, 6.47 mmol, 93.19% yield, 100% purity) as a yellow solid. LCMS: t_{R}=0.359 min, MS (ESI+) m/z=432.9 [M+1]⁺. ¹H NMR (400 MHz, DMSO-d₆) δ=8.75 (s, 1H), 8.63 (d, J=8.4 Hz, 1H), 8.15-8.05 (m, 1H), 8.04-7.98 (m, 1H), 6.62 (s, 1H), 5.17 (s, 2H), 4.64 (q, J=7.2 Hz, 2H), 4.34-4.15 (m, 1H), 4.04-3.80 (m, 2H), 3.65-3.50 (m, 2H), 2.46-2.36 (m, 3H), 2.15-1.91 (m, 4H), 1.75-1.66 (m, 1H), 1.51 (t, J=7.2 Hz, 3H), 1.20 (d, J=6.0 Hz, 3H).

Procedure for preparation of compound 627-16. To a solution of compound 57-15 (20 g, 72.41 mmol, 1 eq) and compound 627-15 (20.23 g, 108.61 mmol, 1.5 eq) in DMA (200 mL) was added DIEA (28.07 g, 217.22 mmol, 37.84 mL, 3 eq). The mixture was stirred at 100° C. for 12 h. The reaction mixture was quenched by addition H₂O (1 L) and filtered to give the crude product. The crude product was triturated with MeCN (100 ml) at 20° C. for 0.5 h to give compound 627-16 (30 g, crude) was obtained as a yellow solid. LCMS: t_{R}=0.439 min, MS (ESI+) m/z=443.0 [M+1]⁺. ¹H NMR (400 MHz, CDCl₃) δ=7.97 (s, 1H), 7.62 (dd, J=8.4, 7.2 Hz, 1H), 7.44 (d, J=7.2 Hz, 1H), 7.17 (d, J=8.4 Hz, 1H), 4.97 (dd, J=12.4, 5.2 Hz, 1H), 3.66 (t, J=4.8 Hz, 4H), 3.29 (q, J=4.4 Hz, 4H), 2.94-2.68 (m, 3H), 2.17-2.10 (m, 1H), 1.50 (s, 9H).

Procedure for preparation of compound 627-17. To a solution of compound 627-16 (28 g, 63.28 mmol, 1 eq) in DCM (100 mL) was added HCl/dioxane (4 M, 100 mL, 6.32 eq), the reaction mixture as stirred at 20° C. for 1 h. The reaction mixture was concentrated under reduced pressure to give compound 627-17 (33.7 g, crude, HCl) was obtained as a yellow solid. LCMS: t_{R}=0.223 min, MS (ESI+) m/z=342.9 [M+1]⁺. ¹H NMR (400 MHz, DMSO-d₆) δ=11.10 (s, 1H), 9.66 (s, 2H), 7.75 (dd, J=8.4, 7.2 Hz, 1H), 7.42 (t, J=8.4 Hz, 2H), 5.11 (dd, J=12.8, 5.2 Hz, 1H), 3.52 (s, 4H), 3.24 (s, 4H), 2.95-2.85 (m, 1H), 2.64-2.52 (m, 2H), 2.05-1.98 (m, 1H).

Procedure for preparation of compound 627-19. To a solution of compound 627-17 (1 g, 2.92 mmol, 1 eq) and compound 627-18 (608.18 mg, 3.51 mmol, 528.85 uL, 60% purity, 1.2 eq) in HOAc (1 mL) and DMAc (10 mL) was added borane;2-methylpyridine (312.43 mg, 2.92 mmol, 1 eq) and the mixture was stirred at 25° C. for 2 h. The reaction mixture was concentrated under reduced pressure to give a residue. The residue was purified by prep-HPLC (10%-40% ACN in water (0.225% FA), 10 min) and lyophilized to give compound 627-19 (500 mg, 1.05 mmol, 35.92% yield, FA) as a yellow solid. LCMS: t_{R}=0.262 min, MS (ESI+) m/z=431.0 [M+1]⁺. ¹H NMR (400 MHz, DMSO-d₆) δ=11.10 (s, 1H), 8.14 (s, 1H), 7.76 (t, J=7.8 Hz, 1H), 7.42 (dd, J=19.6, 7.6 Hz, 2H), 5.11 (dd, J=12.6, 5.2 Hz, 1H), 3.37 (s, 10H), 2.92-2.83 (m, 1H), 2.73-2.52 (m, 6H), 2.09-2.01 (m, 1H).

Procedure for preparation of compound 627-20. A mixture of compound 627-19 (300 mg, 696.94 μmol, 1 eq) in HCl/dioxane (4 M, 1.50 mL, 8.61 eq) was stirred at 50° C. for 2 h. The reaction mixture was concentrated under reduced pressure to give compound 627-20 (200 mg, crude) as a yellow solid. LCMS: t_{R}=0.221 min, MS (ESI+) m/z=402.9 [M+1]⁺. ¹H NMR (400 MHz, DMSO-d₆) δ=11.09 (s, 1H), 9.64 (s, 1H), 7.76-7.66 (m, 1H), 7.43-7.30 (m, 2H), 5.09 (dd, J=12.8, 5.2 Hz, 1H), 3.57 (s, 4H), 3.32-3.23 (m, 4H), 2.92-2.82 (m, 1H), 2.66 (d, J=4.4 Hz, 4H), 2.63-2.53 (m, 2H), 2.08-2.00 (m, 1H).

Procedure for preparation of MS-6-027. To a solution of compound 627-20 (80 mg, 208.12 μmol, 1 eq) and compound 624-5 (90.02 mg, 208.12 μmol, 1 eq) in DMAc (1 mL) was added 2-Pic-BH₃ (22.26 mg, 208.12 μmol, 1 eq) and the mixture was stirred at 25° C. for 2 h. The reaction mixture was filtered. The residue was purified by prep-HPLC (41%-71% ACN in water (10 mM NH₄HCO₃), 8 min) and lyophilized to give MS-6-027 (10.07 mg, 12.15 μmol, 5.84% yield, 96.65% purity) as a yellow solid. LCMS: t_{R}=0.383 min, MS (ESI+) m/z=801.5 [M+1]⁺. ¹H NMR (400 MHz, DMSO-d₆) δ=11.22-10.78 (m, 1H), 8.75 (s, 1H), 8.61 (d, J=8.0 Hz, 1H), 8.13-7.90 (m, 2H), 7.72-7.57 (m, 1H), 7.40-7.27 (m, 1H), 7.08-6.94 (m, 1H), 6.66-6.51 (m, 1H), 5.38-5.19 (m, 2H), 5.14-4.95 (m, 1H), 4.69 (d, J=7.2 Hz, 2H), 4.32-4.08 (m, 1H), 3.74 (s, 2H), 3.62-3.47 (m, 1H), 3.31 (s, 4H), 3.02 (s, 4H), 2.92-2.78 (m, 1H), 2.71-2.54 (m, 2H), 2.44-2.39 (m, 4H), 2.33 (s, 4H), 2.09-1.94 (m, 4H), 1.69 (d, J=1.6 Hz, 1H), 1.57-1.45 (m, 3H), 1.20 (d, J=6.0 Hz, 3H).

### Example 56

### Synthesis of MS-6-028

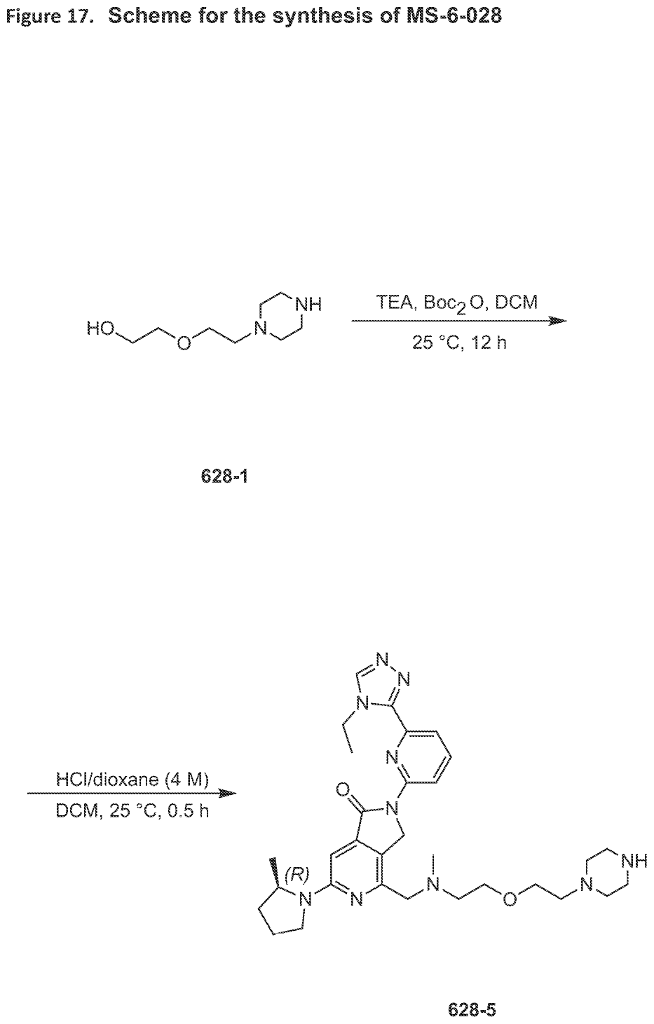

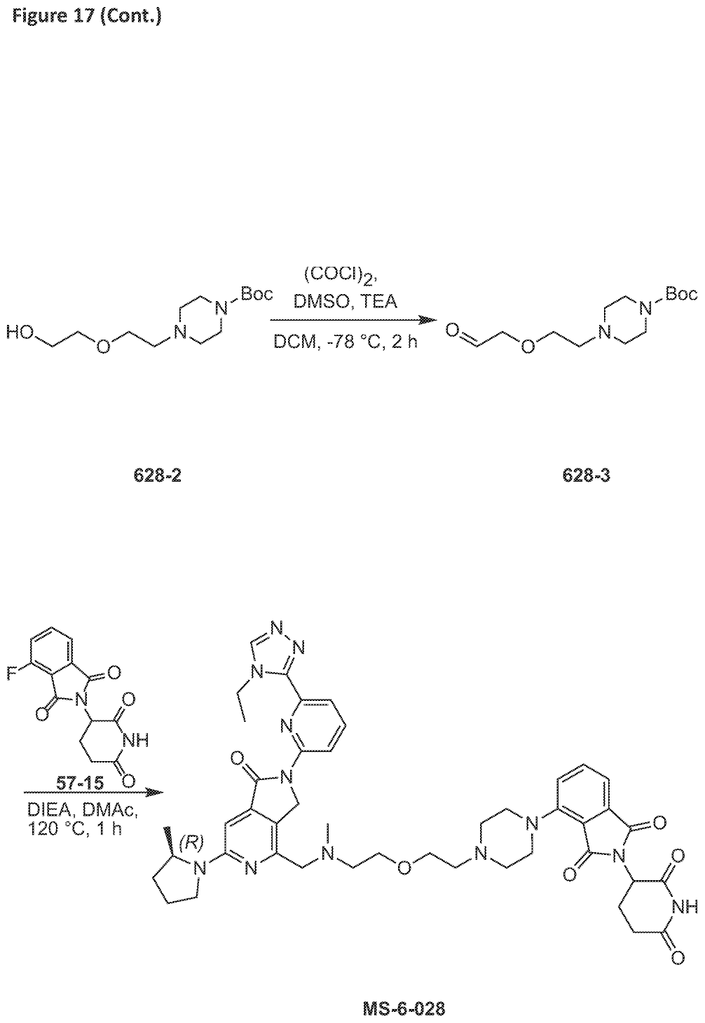

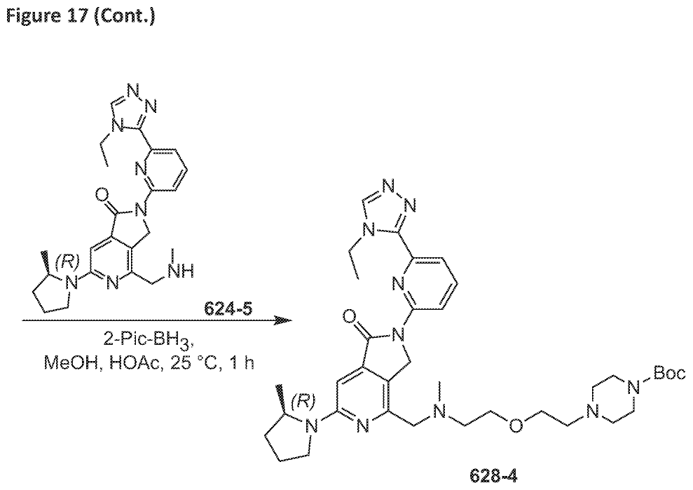

The reaction scheme for the synthesis of MS-6-028 is shown in FIG. 17.

The procedure for preparing compound 624-5 is described in the synthesis of MS-6-027.

Procedure for preparation of compound 628-2. To a solution of compound 628-1 (5 g, 28.70 mmol, 4.72 mL, 1 eq) in DCM (30 mL) was added TEA (5.81 g, 57.39 mmol, 7.99 mL, 2 eq) and Boc₂O (6.89 g, 31.57 mmol, 7.25 mL, 1.1 eq) and the mixture was stirred at 25° C. for 12 h. The mixture was concentrated in vacuum to give a residue. The residue was purified by flash silica gel chromatography (SiO₂, Petroleum ether/Ethyl acetate=100/1 to 0/1) to give compound 628-2 (6 g, 21.87 mmol, 76.21% yield) as a yellow oil. LCMS: t_{R}=0.208 min, MS (ESI+) m/z=275.2 [M+1]⁺. ¹H NMR (400 MHz, CDCl₃) δ=3.65-3.52 (m, 6H), 3.42-3.36 (m, 4H), 2.56-2.50 (m, 2H), 2.45-2.36 (m, 4H), 1.39 (s, 9H)

Procedure for preparation of compound 628-3. To a solution of DMSO (711.97 mg, 9.11 mmol, 711.97 uL, 5 eq) in DCM (10 mL) was added a mixture of (COCl)₂ (508.90 mg, 4.01 mmol, 350.96 uL, 2.2 eq) in DCM (10 mL) and stirred at −78° C. for 30 min and then compound 628-2 (500 mg, 1.82 mmol, 1 eq) in DCM (10 mL) was added, then TEA (1.48 g, 14.58 mmol, 2.03 mL, 8 eq) was added and the resulting mixture was stirred at −78° C. for another 1.5 h. The reaction mixture quenched by H₂O (50 mL) and extracted with DCM (200 mL), the organic layers was concentrated under reduced pressure to give compound 628-3 (450 mg, 1.65 mmol, 90.67% yield) as a yellow oil.

Procedure for preparation of compound compound 628-4. To a solution of compound 624-5 (300 mg, 639.68 μmol, 1 eq, HCl) and compound 628-3 (261.32 mg, 959.53 μmol, 1.5 eq) in MeOH (1 mL) and HOAc (0.1 mL) was added 2-Pic-BH₃ (68.42 mg, 639.68 μmol, 1 eq) and the mixture was stirred at 25° C. for 1 h. The reaction mixture was concentrated under reduced pressure to give a residue. The residue was purified by prep-HPLC (10-40% ACN in water (0.225% FA), 10 min) and lyophilized to give compound 628-4 (250 mg, 362.92 μmol, 56.73% yield, 100% purity) as a yellow oil. LCMS: t_{R}=0.358 min, MS (ESI+) m/z=689.4 [M+1]⁺

Procedure for preparation of compound 628-5. To a solution of compound 628-4 (240 mg, 348.40 μmol, 1 eq) in DCM (2 mL) was added HCl/dioxane (4 M, 1 mL, 11.48 eq) and the mixture was stirred at 25° C. for 0.5 h. The residue was quenched by NaHCO₃ (aq) to adjust the pH to 9 and extracted with DCM (50 mL×2), the combined organic layers were concentrated under reduced pressure to give compound 628-5 (200 mg, 203.82 μmol, 58.50% yield, 60% purity) as a brown solid. LCMS: t_{R}=0.312 min, MS (ESI+) m/z=589.3 [M+1]⁺

Procedure for preparation of MS-6-028. To a solution of compound 628-5 (100 mg, 103.61 μmol, 61% purity, 1 eq) and compound 57-15 (28.62 mg, 103.61 μmol, 1 eq) in DMAc (1 mL) was added DIEA (13.39 mg, 103.61 μmol, 18.05 uL, 1 eq) and the mixture was stirred at 120° C. for 1 h. The residue was purified by prep-HPLC (7%-37% ACN in water (0.225% FA), 10 min) and lyophilized to give MS-6-028 (26.40 mg, 29.54 μmol, 28.51% yield, 99.7% purity, FA) as a yellow solid. LCMS: t_{R}=1.409 min, MS (ESI+) m/z=845.3 [M+1]⁺. ¹H NMR (400 MHz, DMSO-d₆) δ=11.08 (s, 1H), 8.74 (s, 1H), 8.59 (d, J=8.0 Hz, 1H), 8.16 (s, 2H), 8.08-8.01 (m, 1H), 8.01-7.92 (m, 1H), 7.67 (t, J=7.6 Hz, 1H), 7.34 (d, J=7.2 Hz, 1H), 7.19 (d, J=8.4 Hz, 1H), 6.57 (s, 1H), 5.21 (s, 2H), 5.07 (dd, J=12.8, 5.2 Hz, 1H), 4.67 (q, J=7.2 Hz, 2H), 4.21-4.15 (m, 1H), 3.75 (s, 2H), 3.51 (d, J=5.2 Hz, 2H), 3.42-3.39 (m, 2H), 3.33-3.27 (m, 2H), 3.10 (s, 4H), 2.91-2.83 (m, 1H), 2.62-2.53 (m, 4H), 2.38 (s, 4H), 2.35-2.27 (m, 5H), 2.11-1.92 (m, 4H), 1.66 (d, J=1.6 Hz, 1H), 1.50 (t, J=7.2 Hz, 3H), 1.17 (d, J=6.0 Hz, 3H).

### Example 57

Synthetic Route for MS-6-029

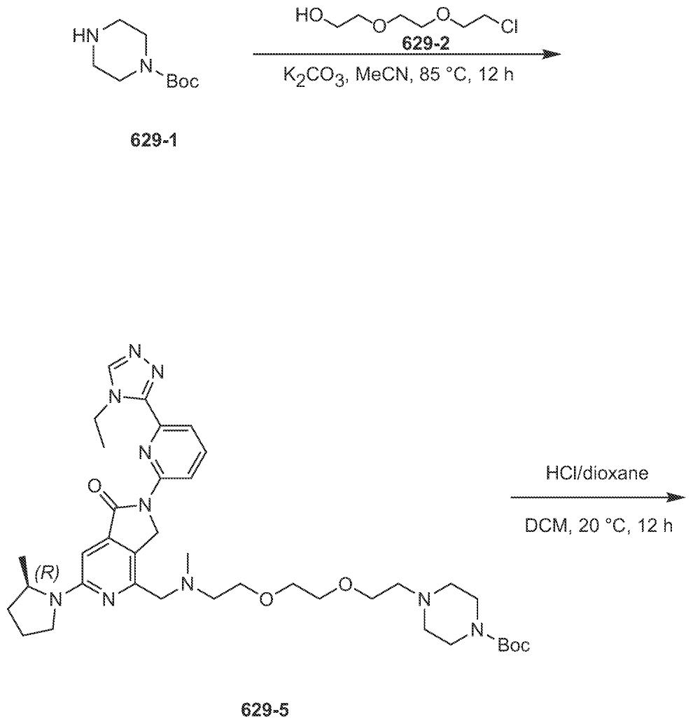

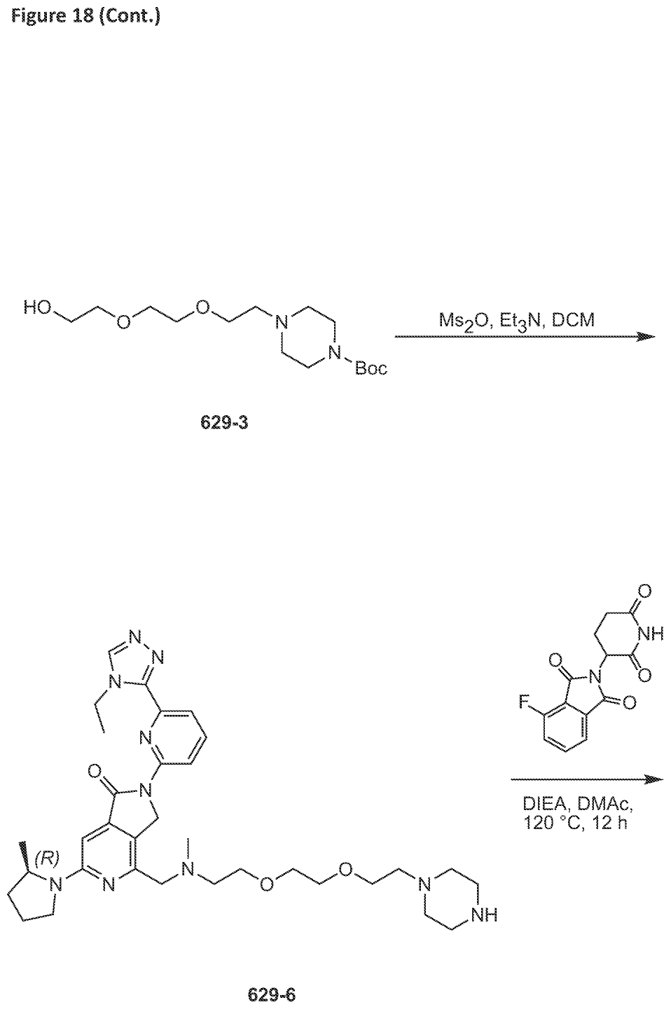

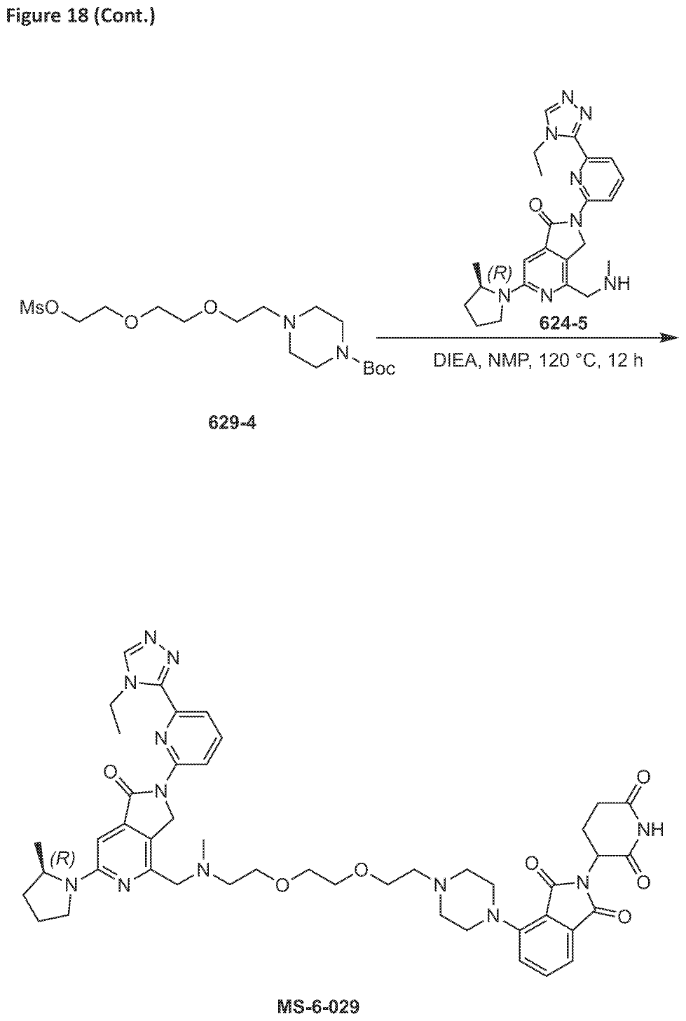

The reaction scheme for the synthesis of MS-6-029 is shown in FIG. 18.

The procedure for preparing compound 624-5 is described in the synthesis of MS-6-027.

Procedure for preparation of compound 629-3. To a solution of compound 629-1 (8 g, 42.95 mmol, 1 eq), compound 629-2 (10.14 g, 60.13 mmol, 1.4 eq) in MeCN (50 mL) was added K₂CO₃ (17.81 g, 128.86 mmol, 3 eq). Then the mixture was stirred at 85° C. for 12 h. The mixture was quenched with water (50 mL) and extracted with ethyl acetate (50 mL×3), the combined organic phase was dried over anhydrous sodium sulfate, filtered and concentrated to give a residue. The residue was purified by flash silica gel chromatography (SiO2, DCM/MeOH=100/1 to 10/1) to give compound 3629- (10 g, 31.41 mmol, 73.12% yield) as yellow oil. ¹H NMR (400 MHz, DMSO-d₆) δ=4.59-4.54 (m, 1H), 3.52-3.39 (m, 12H), 3.30-3.25 (m, 4H), 2.37-2.32 (m, 4H), 1.39 (s, 9H).

Procedure for preparation of compound 629-4. To a solution of compound 629-3 (1 g, 3.14 mmol, 1 eq) in DCM (10 mL) was added Et₃N (3.18 g, 31.41 mmol, 4.37 mL, 10 eq), Ms₂O (4.38 g, 25.13 mmol, 8 eq) at 0° C. Then the mixture was stirred at 25° C. for 1 h. The mixture was concentrated in vacuo to give compound 629-4 (1 g, crude) as yellow oil. LCMS: t_{R}=0.266 min, MS (ESI+) m/z=397.1[M+1]⁺

Procedure for preparation of compound 629-5. To a solution of compound 629-4 (1 g, 2.13 mmol, 1 eq, HCl), compound 624-5 (1.01 g, 2.56 mmol, 1.2 eq) in NMP (6 mL) was added DIEA (1.38 g, 10.66 mmol, 1.86 mL, 5 eq). Then the mixture was stirred at 120° C. for 12 h. The mixture was filtered and purified by prep-HPLC (9%-39% ACN in water (0.225% FA), 10 min) to give compound 629-5 (145 mg, 197.84 μmol, 9.28% yield) as brown oil. LCMS: t_{R}=0.371 min, MS (ESI+) m/z=733.3[M+1]⁺. ¹H NMR (400 MHz, DMSO-d₆) δ=8.75 (s, 1H), 8.63 (d, J=8.4 Hz, 1H), 8.08 (t, J=7.6 Hz, 2H), 8.01 (d, J=7.2 Hz, 1H), 6.60 (s, 1H), 5.19 (s, 2H), 4.66 (q, J=7.2 Hz, 2H), 4.25-4.15 (m, 1H), 3.82-3.76 (m, 2H), 3.58-3.49 (m, 8H), 3.22 (s, 13H), 2.34 (s, 3H), 2.12-1.91 (m, 4H), 1.69 (s, 1H), 1.53-1.46 (m, 3H), 1.19 (d, J=6.0 Hz, 3H).

Procedure for preparation of compound 629-6. To a solution of compound 629-5 (145 mg, 197.84 μmol, 1 eq) in DCM (1 mL) was added HCl/dioxane (4 M, 247.30 uL, 5 eq) at 0° C. Then the mixture was stirred at 25° C. for 1 h. The mixture was quenched with saturated solution of NaHCO₃ (2 mL) and extracted with dichloromethane/methanol (10/1), the combined organic phase was dried over anhydrous sodium sulfate, filtered and concentrated to give compound 629-6 (91 mg, crude) as brown oil. LCMS: t_{R}=0.305 min, MS (ESI+) m/z=633.4[M+1]⁺

Procedure for preparation of compound MS-6-029. To a solution of compound 629-6 (91 mg, 143.81 μmol, 1 eq) in NMP (0.5 mL) was added 2-(2,6-dioxopiperidin-3-yl)-4-fluoroisoindoline-1,3-dione (51.64 mg, 186.95 μmol, 1.3 eq) and DIEA (55.76 mg, 431.42 μmol, 75.15 uL, 3 eq). The vial was heated at 120° C. for 12 h. The mixture was filtered and purified by prep-HPLC (11%-31% ACN in water (0.225% FA), 9 min) to give MS-6-029 (35.31 mg, 38.05 μmol, 26.46% yield, 95.8% purity) as yellow solid. LCMS: t_{R}=0.347 min, MS (ESI+) m/z=889.5[M+1]⁺. ¹H NMR (EC8254-41-P1A) (400 MHz, DMSO-d₆) δ=10.97 (s, 1H), 8.61 (s, 1H), 8.46 (d, J=8.0 Hz, 1H), 8.05 (s, 2H), 7.96-7.80 (m, 2H), 7.54 (dd, J=8.0, 7.2 Hz, 1H), 7.21 (d, J=7.2 Hz, 1H), 7.12 (d, J=8.6 Hz, 1H), 6.40 (s, 1H), 5.01 (s, 2H), 4.96 (dd, J=12.8, 5.6 Hz, 1H), 4.53 (q, J=7.2 Hz, 2H), 4.09-3.97 (m, 4H), 3.59 (s, 2H), 3.44-3.31 (m, 4H), 3.13-3.10 (m, 1H), 3.30-3.00 (m, 11H), 2.75 (m, 1H), 2.50-2.44 (m, 2H), 2.24 (t, J=5.6 Hz, 2H), 2.16 (s, 3H), 1.94-1.75 (m, 4H), 1.53 (s, 1H), 1.36 (t, J=7.2 Hz, 3H), 1.03 (d, J=6.0 Hz, 3H).

### Example 58

### Synthesis of MS-6-037

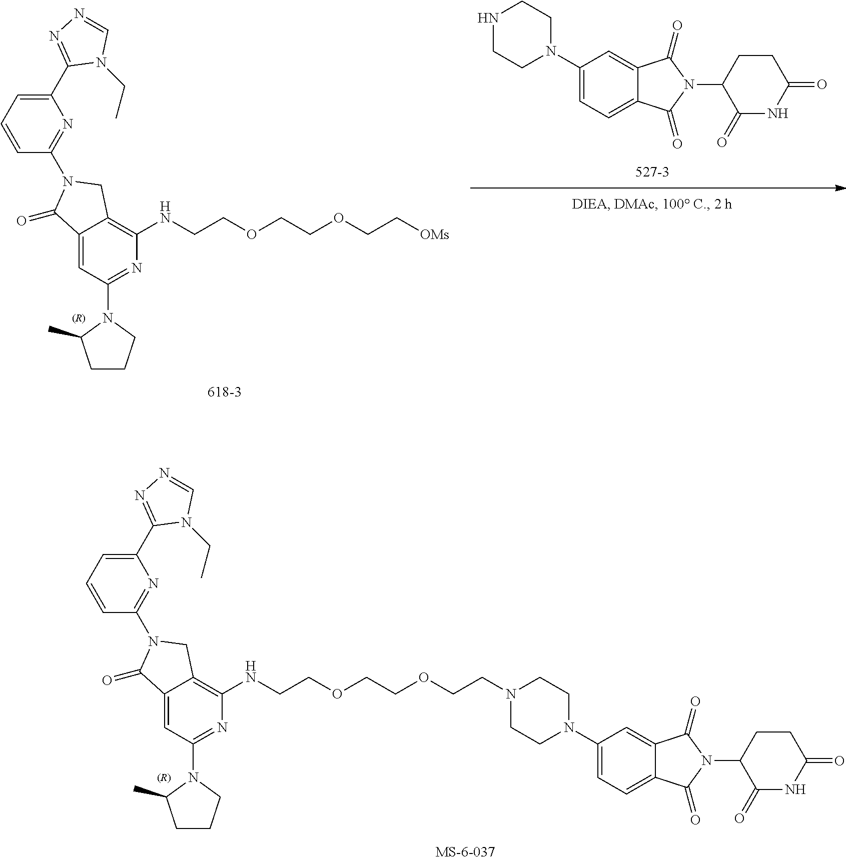

The procedure for preparing compound 618-3 is described in the synthesis of MS-6-018.

The procedure for preparing compound 527-3 is described in the synthesis of MS-6-046.

Procedure for preparation of compound MS-6-037. To a solution of compound 618-3 (130 mg, 211.48 μmol, 1 eq) in NMP (1 mL) was added compound 527-3 (108.60 mg, 317.22 μmol, 1.5 eq), DIEA (82.00 mg, 634.44 μmol, 110.51 uL, 3 eq). Then the mixture was stirred at 120° C. for 12 h. The reaction was monitored by LCMS, LCMS showed compound 618-3 was consumed and the desired mass was detected. The mixture was filtered and purified by prep-HPLC (25%-45% ACN in water (0.225% FA), 9 min) to give MS-6-037 (24.54 mg, 25.70 μmol, 12.15% yield, 95% purity, FA) as yellow solid. LCMS: t_{R}=0.374 min, MS (ESI+) m/z=861.4[M+1]⁺. ¹H NMR (400 MHz, DMSO-d₆) δ=11.07 (s, 1H), 8.73 (s, 1H), 8.55 (d, J=8.4 Hz, 1H), 8.16 (s, 1H), 8.03 (t, J=8.0 Hz, 1H), 7.92 (d, J=7.6 Hz, 1H), 7.61 (d, J=8.4 Hz, 1H), 7.31-7.22 (m, 1H), 7.16 (dd, J=2.0, 8.6 Hz, 1H), 6.70 (br t, J=5.4 Hz, 1H), 5.85 (s, 1H), 5.06 (dd, J=5.2, 12.8 Hz, 1H), 4.79 (s, 2H), 4.63 (q, J=7.0 Hz, 2H), 4.20-4.12 (m, 1H), 3.67-3.64 (m, 2H), 3.59-3.51 (m, 10H), 3.38-3.20 (m, 10H), 2.88 (ddd, J=5.2, 14.0, 17.4 Hz, 1H), 2.63-2.53 (m, 2H), 2.06-1.87 (m, 4H), 1.70-1.59 (m, 1H), 1.38 (t, J=7.2 Hz, 3H), 1.19 (d, J=6.0 Hz, 3H).

### Example 59

### Synthesis of MS-6-038

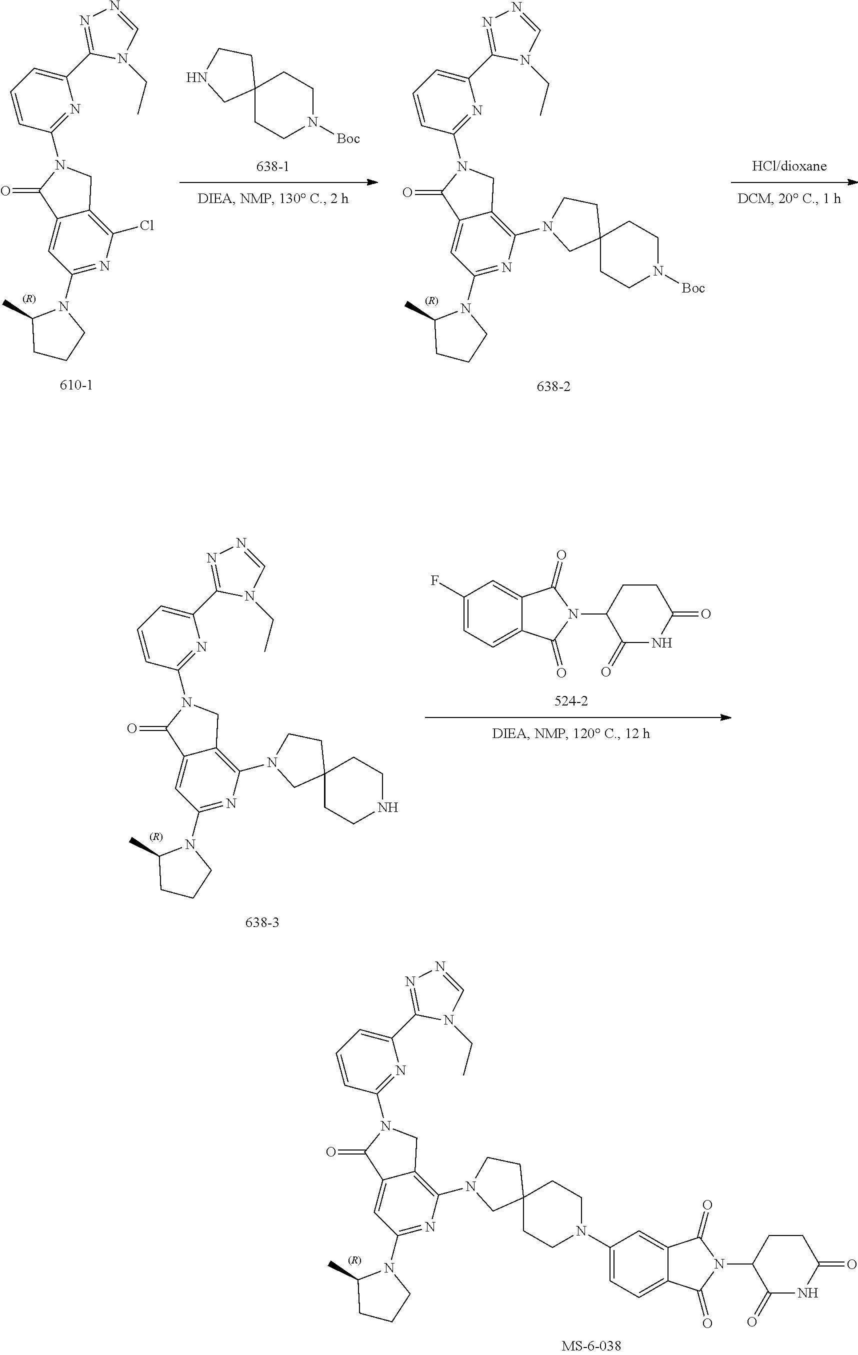

The procedure for preparing compound 610-1 is described in the synthesis of MS-6-006.

Procedure for preparation of 638-2. To a solution of compound 610-1 (300 mg, 707.72 μmol, 1 eq) and compound 638-1 (340.19 mg, 1.42 mmol, 2 eq) in NMP (3 mL) was added DIEA (274.40 mg, 2.12 mmol, 369.81 uL, 3 eq). The mixture was stirred at 130° C. for 2 h by microwave. The residue was purified by prep-HPLC (63%-93% ACN in water (10 mM NH₄HCO₃), 8 min) and lyophilized to give compound 3 (184 mg, 274.16 μmol, 38.74% yield) as a yellow solid. LCMS: t_{R}=0.537 min, MS (ESI+) m/z=627.9 [M+1]⁺. ¹H NMR (400 MHz, DMSO-d₆) δ=8.74 (s, 1H), 8.61 (d, J=8.0 Hz, 1H), 8.06 (t, J=8.0 Hz, 1H), 7.98 (d, J=7.6 Hz, 1H), 5.90 (s, 1H), 5.33 (s, 2H), 4.61 (q, J=7.2 Hz, 2H), 4.15 (s, 1H), 3.72 (d, J=8.4 Hz, 2H), 3.62-3.50 (m, 2H), 3.46 (s, 4H), 3.26 (d, J=9.8 Hz, 2H), 2.05-1.90 (m, 3H), 1.84 (t, J=6.6 Hz, 2H), 1.65 (s, 1H), 1.53-1.43 (m, 7H), 1.40 (s, 9H), 1.20 (d, J=6.0 Hz, 3H).

Procedure for preparation of compound 638-3. To a solution of compound 638-2 (180 mg, 286.73 μmol, 1 eq) in DCM (2 mL) was added HCl/dioxane (4 M, 358.41 μL, 5 eq). The mixture was stirred at 20° C. for 1 h. The reaction mixture was filtered and the filter residue was concentrated under the reduced pressure to give compound 638-3 (84 mg, 138.66 μmol, 48.36% yield) as a red solid. LCMS: t_{R}=0.387 min, MS (ESI+) m/z=528.1 [M+1]⁺

Procedure for preparation of MS-6-038. To a solution of compound 638-3 (84 mg, 159.19 μmol, 1 eq) and compound 524-2 (65.96 mg, 238.79 μmol, 1.5 eq) in NMP (1 mL) was added DIEA (41.15 mg, 318.39 μmol, 55.46 μL, 2 eq). The mixture was stirred at 120° C. for 12 h. The residue was purified by prep-HPLC (58%-88% ACN in water (0.225% FA), 10 min) to give MS-6-038 (27.88 mg, 34.06 μmol, 21.40% yield) as a yellow solid. LCMS: t_{R}=0.516 min, MS (ESI+) m/z=784.0 [M+1]⁺. ¹H NMR (400 MHz, DMSO-d₆) δ=11.08 (s, 1H), 8.69 (s, 1H), 8.61 (dd, J=8.4, 0.8 Hz, 1H), 8.09-8.02 (m, 1H), 7.97 (dd, J=7.6, 0.8 Hz, 1H), 7.68 (d, J=8.6 Hz, 1H), 7.37 (d, J=1.6 Hz, 1H), 7.28 (dd, J=8.6, 2.0 Hz, 1H), 5.91 (s, 1H), 5.35 (s, 2H), 5.07 (dd, J=12.8, 5.4 Hz, 1H), 4.59 (q, J=7.2 Hz, 2H), 4.21-4.11 (m, 1H), 3.85-3.70 (m, 2H), 3.68-3.53 (m, 6H), 3.49-3.44 (m, 1H), 3.26-3.22 (m, 1H), 2.92-2.81 (m, 1H), 2.62-2.54 (m, 2H), 2.01 (dd, J=7.6, 3.6 Hz, 3H), 1.91 (t, J=6.8 Hz, 3H), 1.67 (s, 5H), 1.44 (t, J=7.2 Hz, 3H), 1.20 (d, J=6.4 Hz, 3H).

### Example 60

### Synthesis of MS-6-039

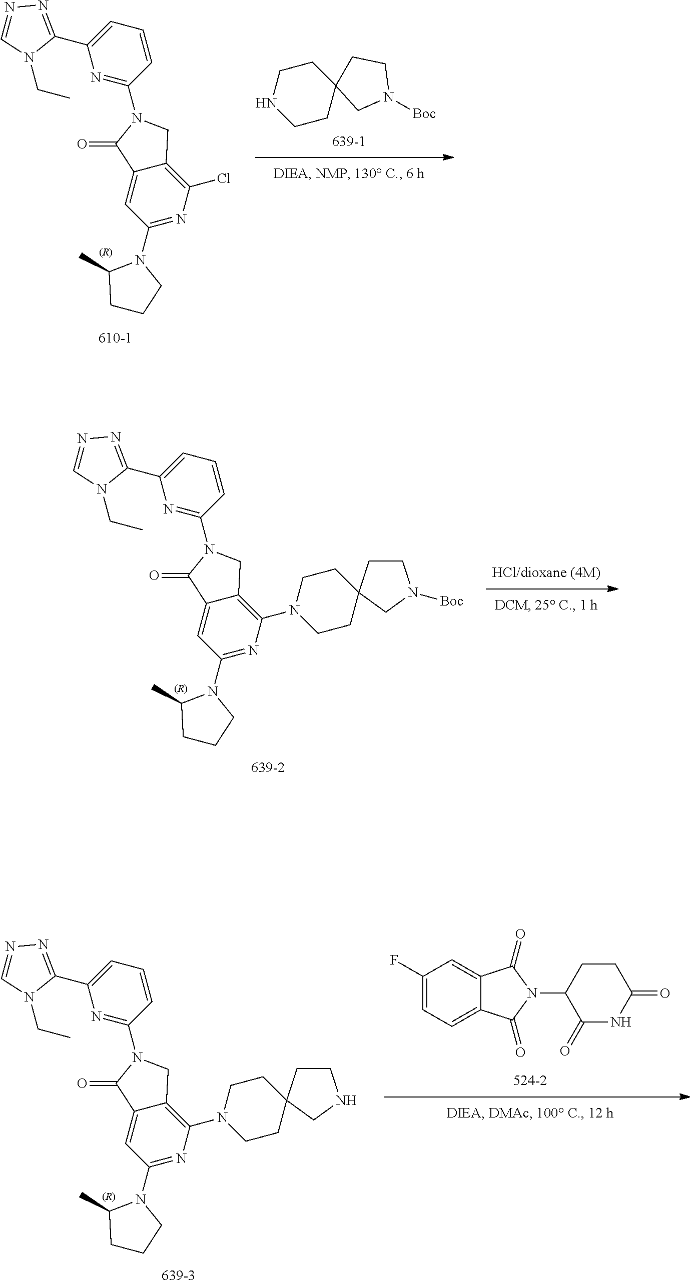

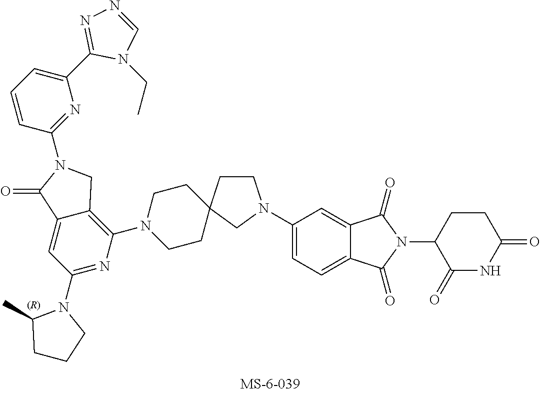

The procedure for preparing compound 610-1 is described in the synthesis of MS-6-006.

Procedure for preparation of compound 639-2. To a solution of compound 610-1 (300 mg, 707.72 μmol, 1 eq) and compound 639-1 (340.19 mg, 1.42 mmol, 2 eq) in NMP (3 mL) was added DIEA (274.40 mg, 2.12 mmol, 369.81 μL, 3 eq). The mixture was stirred at 130° C. for 6 h by microwave. The residue was purified by prep-HPLC (60%-90% ACN in water (10 mM NH₄HCO₃), 8 min) and lyophilized to give compound 639-2 (119 mg, 185.77 μmol, 26.25% yield) as a red solid. LCMS: t_{R}=0.590 min, MS (ESI+) m/z=628.2[M+1]⁺. ¹H NMR (400 MHz, DMSO-d₆) δ=8.73 (s, 1H), 8.61 (d, J=7.6 Hz, 1H), 8.07 (t, J=8.0 Hz, 1H), 7.99 (d, J=7.6 Hz, 1H), 6.06 (s, 1H), 5.17 (s, 2H), 4.61 (q, J=7.2 Hz, 2H), 4.15 (t, J=6.3 Hz, 1H), 3.59 (s, 3H), 3.55-3.43 (m, 2H), 3.31-3.20 (m, 3H), 3.15 (s, 2H), 2.10-1.90 (m, 3H), 1.83-1.72 (m, 2H), 1.70-1.61 (m, 1H), 1.58 (s, 4H), 1.47 (t, J=7.6 Hz, 3H), 1.40 (s, 9H), 1.19 (d, J=6.0 Hz, 3H).

Procedure for preparation of compound 639-3. To a solution of compound 639-2 (110 mg, 175.22 mol, 1 eq) in DCM (1 mL) was added HCl/dioxane (4 M, 131.42 μL, 3 eq). The mixture was stirred at 25° C. for 1 h. The reaction mixture was concentrated in vacuum and the reaction mixture was added water and NaHCO₃ (aq) until pH around 8, then extracted with EtOAC (10 mL×3). The combined organic phase was washed with brine (10 mL×3), dried over anhydrous Na₂SO₄, filtered and concentrated in vacuo to give compound 639-3 (90 mg, 163.74 μmol, 97.83% yield) as a yellow solid. LCMS: t_{R}=0.408 min, MS (ESI+) m/z=528.0[M+1]⁺

Procedure for preparation of MS-6-039. To a solution of compound 639-3 (90 mg, 170.56 μmol, 1 eq) and compound 524-2 (47.11 mg, 170.56 μmol, 1 eq) in DMAc (2 mL) was added DIEA (44.09 mg, 341.13 μmol, 59.42 μL, 2 eq). The mixture was stirred at 100° C. for 12 h. The residue was purified by prep-HPLC (61%-91% ACN in water (0.225% FA), 58 min) and lyophilized to give MS-6-039 (36.07 mg, 43.81 μmol, 25.68% yield) as a yellow solid. LCMS: t_{R}=0.552 min, MS (ESI+) m/z=784.36[M+1]⁺. ¹H NMR (400 MHz, DMSO-d₆) δ=11.08 (s, 1H), 8.71 (s, 1H), 8.62 (d, J=8.0 Hz, 1H), 8.08 (t, J=7.0 Hz, 1H), 7.99 (d, J=7.8 Hz, 1H), 7.66 (d, J=8.4 Hz, 1H), 6.98 (s, 1H), 6.85 (dd, J=2.0, 8.6 Hz, 1H), 6.08 (s, 1H), 5.19 (s, 2H), 5.07 (dd, J=5.4, 12.8 Hz, 1H), 4.61 (q, J=7.0 Hz, 2H), 4.17 (t, J=5.8 Hz, 1H), 3.80-3.70 (m, 2H), 3.66-3.47 (m, 5H), 3.45-3.39 (m, 2H), 3.29 (s, 1H), 2.95-2.83 (m, 1H), 2.63-2.54 (m, 2H), 2.10-1.93 (m, 6H), 1.69 (s, 5H), 1.46 (t, J=7.6 Hz, 3H), 1.20 (d, J=6.2 Hz, 3H).

### Example 61

### Synthesis of MS-6-040

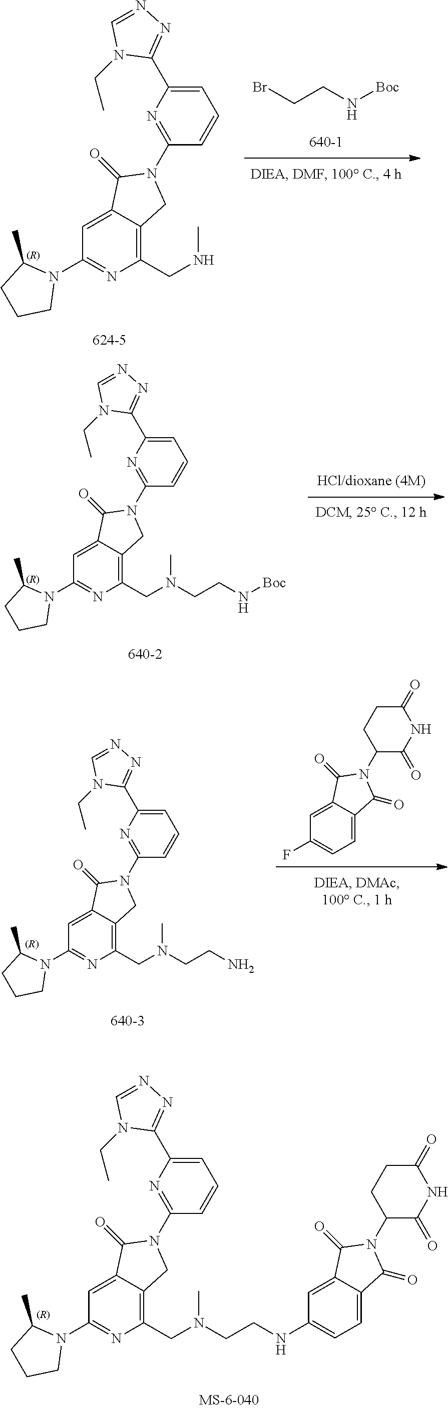

The procedure for preparing compound 624-5 is described in the synthesis of MS-6-027.

Procedure for preparation of compound 640-2. To a solution of compound 624-5 (1 g, 2.13 mmol, 1 eq, HCl) and compound 640-1 (716.75 mg, 3.20 mmol, 1.5 eq) in DMF (10 mL) was added DIEA (826.75 mg, 6.40 mmol, 1.11 mL, 3 eq) and the mixture was stirred at 100° C. for 4 h. The mixture was concentrated in vacuum to give a residue. The residue was purified by prep-HPLC (10%-40% ACN in water (0.225% FA), 10 min) and lyophilized to give compound 640-2 (300 mg, 510.68 μmol, 23.95% yield, 98% purity) as a brown solid. LCMS: t_{R}=0.407 min, MS (ESI+) m/z=576.2 [M+1]⁺

Procedure for preparation of compound 640-3. To a solution of compound 640-2 (300 mg, 521.10 μmol, 1 eq) in DCM (3 mL) was added HCl/dioxane (4 M, 130.28 uL, 1 eq) and the mixture was stirred at 25° C. for 12 h. The reaction mixture was basified with NaHCO₃ (50 mL) and extracted with DCM/MeOH (4/1, 40 mL×2), the combined organic layers were concentrated under reduced pressure to give compound 640-3 (180 mg, 378.48 μmol, 72.63% yield) as a brown solid. LCMS: t_{R}=0.300 min, MS (ESI+) m/z=476.2 [M+1]⁺

Procedure for preparation of MS-6-040. To a solution of compound 640-3 (60 mg, 117.18 μmol, 1 eq, HCl) and 2-(2,6-dioxopiperidin-3-yl)-5-fluoroisoindoline-1,3-dione (32.37 mg, 117.18 μmol, 1 eq) in DMAc (1 mL) was added DIEA (45.43 mg, 351.53 μmol, 61.23 uL, 3 eq) and the mixture was stirred at 100° C. for 1 h. The residue was purified by prep-HPLC (12%-42% ACN in water (0.225% FA), 10 min) and lyophilized to give MS-6-040 (14.14 mg, 18.02 μmol, 15.38% yield, 99.11% purity, FA) as a yellow solid. LCMS: t_{R}=1.456 min, MS (ESI+) m/z=732.3 [M+1]⁺. ¹H NMR (400 MHz, DMSO-d₆) δ=11.04 (s, 1H), 8.68 (s, 1H), 8.57 (d, J=8.4 Hz, 1H), 8.30-8.23 (m, 1H), 8.12-8.02 (m, 1H), 8.01-7.97 (m, 1H), 7.27 (d, J=8.0 Hz, 1H), 6.88 (t, J=5.2 Hz, 1H), 6.71 (d, J=1.2 Hz, 1H), 6.58 (dd, J=8.4, 2.0 Hz, 1H), 6.47 (s, 1H), 5.11 (s, 2H), 5.00 (dd, J=12.8, 5.6 Hz, 1H), 4.58 (q, J=7.2 Hz, 2H), 4.17 (t, J=5.6 Hz, 1H), 3.80-3.67 (m, 2H), 3.55-3.44 (m, 2H), 3.26 (s, 2H), 2.94-2.84 (m, 1H), 2.66-2.54 (m, 4H), 2.40 (s, 3H), 2.10-1.94 (m, 4H), 1.69 (d, J=2.0 Hz, 1H), 1.47 (t, J=7.2 Hz, 3H), 1.19 (d, J=6.0 Hz, 3H)

### Example 62

### Synthesis of MS-6-041

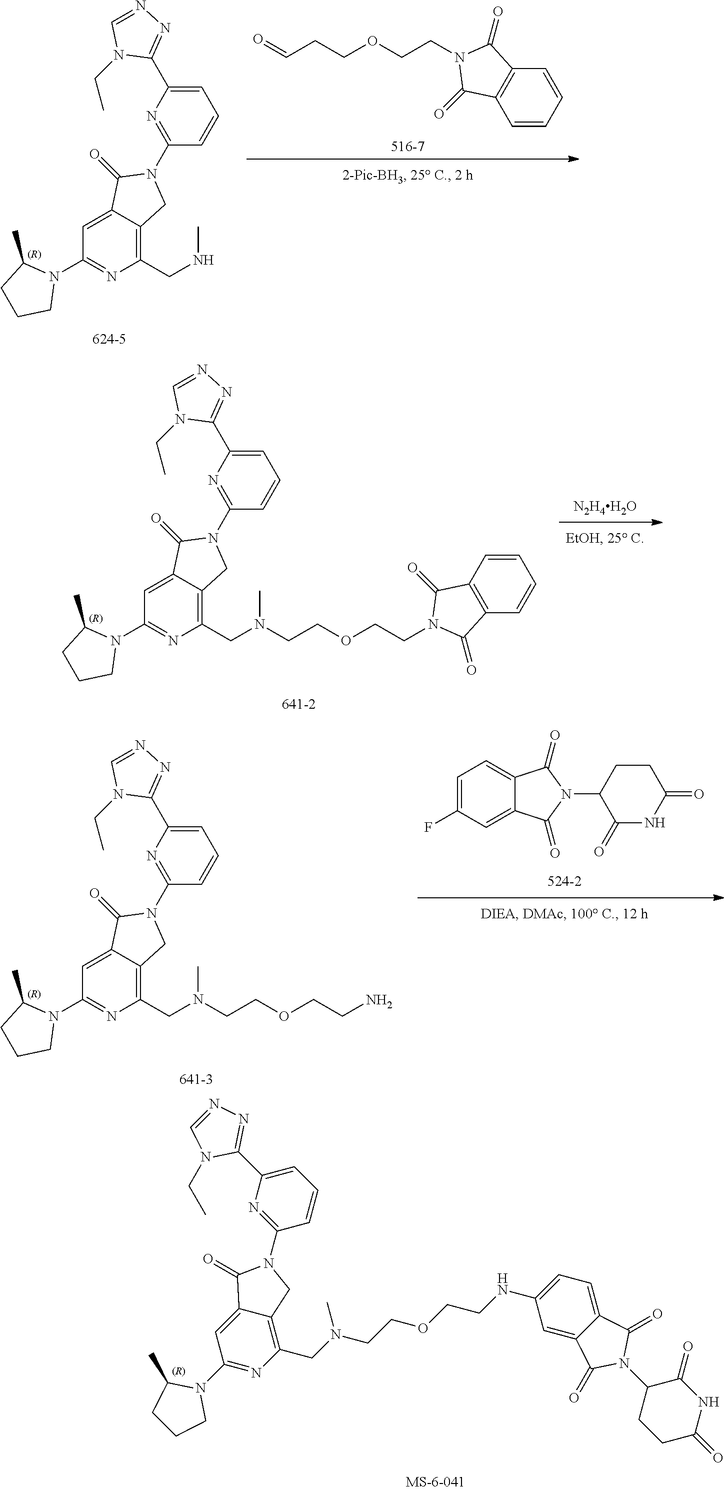

The procedure for preparing compound 624-5 is described in the synthesis of MS-6-027.

The procedure for preparing compound 516-7 is described in the synthesis of MS-5-016.

Procedure for preparation of compound 641-2. To a solution of compound 624-5 (300 mg, 693.61 μmol, 1 eq) and compound 516-7 (566.17 mg, 2.43 mmol, 3.5 eq) in MeOH (2 mL) was added borane;2-methylpyridine (148.38 mg, 1.39 mmol, 2 eq). The mixture was stirred at 25° C. for 2 h. The reaction was monitored by LCMS, LCMS showed compound 624-5 was consumed and the desired mass was detected. The mixture was filtered and purified by prep-HPLC (45%-75% ACN in water (10 mM NH₄HCO₃), 8 min) to give compound 641-2 (176 mg, 235.66 μmol, 33.98% yield, 87% purity) as yellow solid. LCMS: t_{R}=0.391 min, MS (ESI+) m/z=650.2[M+1]⁺. ¹H NMR (400 MHz, DMSO-d₆) δ=8.70 (s, 1H), 8.56 (d, J=8.4 Hz, 1H), 8.08-7.94 (m, 2H), 7.71-7.66 (m, 2H), 7.65-7.59 (m, 2H), 6.51 (s, 1H), 4.99 (s, 2H), 4.59 (q, J=7.2 Hz, 2H), 4.18-4.10 (m, 2H), 3.68-3.51 (m, 10H), 2.16 (s, 3H), 2.07-1.91 (m, 3H), 1.67 (br s, 1H), 1.42 (t, J=7.2 Hz, 3H), 1.16 (d, J=6.3 Hz, 3H).

Procedure for preparation of compound 641-3. To a solution of compound 641-2 (100 mg, 153.91 μmol, 1 eq) in EtOH (1 mL) was added N₂H₄·H₂O (38.52 mg, 769.54 μmol, 37.40 uL, 5 eq). Then the mixture was stirred at 25° C. for 1 h. The reaction was monitored by LCMS, LCMS showed compound 641-2 was consumed and the desired mass was detected. The mixture was concentrated to give a residue to give compound 641-3 (79 mg, crude) as yellow solid. LCMS: t_{R}=0.312 min, MS (ESI+) m/z=520.3[M+1]⁺

Procedure for preparation of compound MS-6-041. To a solution of compound 641-3 (79 mg, 152.03 μmol, 1 eq) and 2-(2,6-dioxopiperidin-3-yl)-5-fluoroisoindoline-1,3-dione (54.59 mg, 197.64 μmol, 1.3 eq) in NMP (1 mL) was added DIEA (58.95 mg, 456.08 μmol, 79.44 uL, 3 eq).

Then the mixture was stirred at 120° C. for 12 h. The reaction was monitored by LCMS, LCMS showed compound 641-3 was consumed and the desired mass was detected. The mixture was filtered and purified by prep-HPLC (17%-47% ACN in water (0.225% FA), 9 min) to give MS-6-041 (176 mg, 235.66 μmol, 33.98% yield, 87% purity) as yellow solid. LCMS: t_{R}=0.367 min, MS (ESI+) m/z=776.3[M+1]⁺. ¹H NMR (400 MHz, DMSO-d₆) δ=11.06 (br s, 1H), 8.70 (s, 1H), 8.55 (d, J=8.2 Hz, 1H), 8.02 (t, J=7.8 Hz, 1H), 7.95 (d, J=7.6 Hz, 1H), 7.36 (d, J=8.2 Hz, 1H), 6.98 (br t, J=5.2 Hz, 1H), 6.80-6.74 (m, 1H), 6.64 (br d, J=8.8 Hz, 1H), 6.58 (s, 1H), 5.13 (s, 2H), 5.02 (dd, J=5.4, 12.8 Hz, 1H), 4.64 (q, J=7.0 Hz, 2H), 4.23-4.15 (m, 1H), 3.74 (s, 2H), 3.59-3.49 (m, 5H), 3.18 (br d, J=5.2 Hz, 2H), 2.95-2.83 (m, 1H), 2.59 (br dd, J=5.4, 11.4 Hz, 4H), 2.28 (s, 2H), 2.08 (s, 3H), 2.06-1.93 (m, 3H), 1.69 (br s, 1H), 1.51-1.40 (m, 3H), 1.19 (d, J=6.0 Hz, 3H).

### Example 63

### Synthesis of MS-6-043

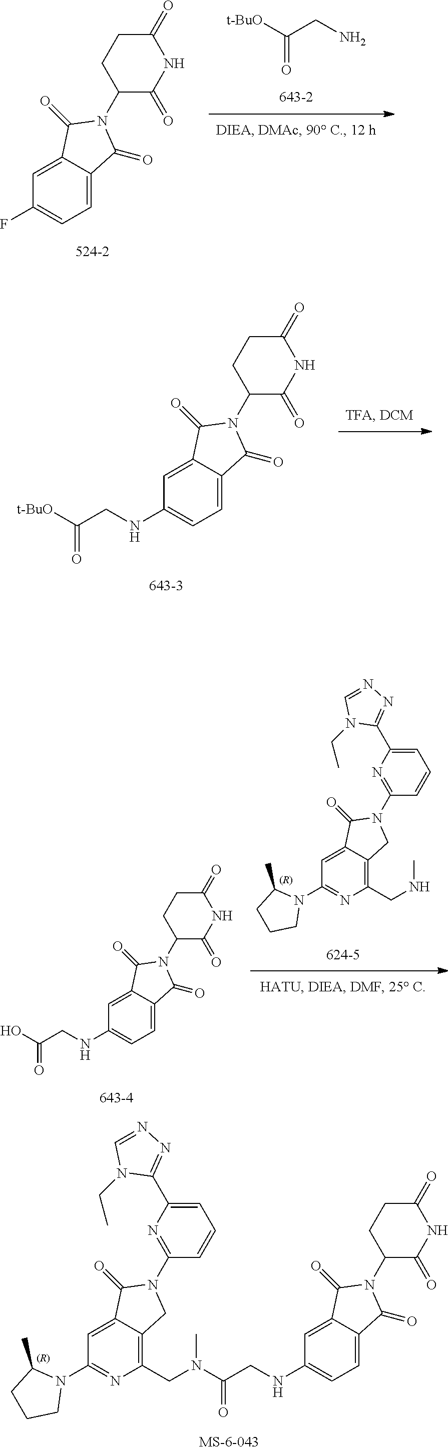

The procedure for preparing compound 624-5 is described in the synthesis of MS-6-027.

Procedure for preparation of compound 643-3. To a solution of compound 643-2 (712.33 mg, 5.43 mmol, 1.5 eq) in DMAc (30 mL) was added DIEA (1.40 g, 10.86 mmol, 1.89 mL, 3 eq), compound 524-2 (1 g, 3.62 mmol, 1 eq). The mixture was stirred at 90° C. for 12 h under an N₂ atmosphere. The reaction was monitored by LCMS, LCMS showed compound 643-1 was consumed and the desired mass was detected. After cooling, the mixture was poured onto half-saturated brine (100 mL), and it was extracted with EtOAc (50 mL×3). The combined organic layers were further washed with brine (50 mL). The organic layers were dried over Na₂SO₄, filtered and concentrated in vacuo. The residue was purified by flash silica gel chromatography (0˜60% Ethylacetate/Petroleum ether) to give compound 643-3 (450 mg, 1.16 mmol, 32.09% yield) as yellow solid. LCMS: t_{R}=0.408 min, MS (ESI+) m/z=388.1[M+1]⁺. ¹H NMR ¹H NMR (400 MHz, DMSO-d₆) δ=11.07 (s, 1H), 7.60 (d, J=8.4 Hz, 1H), 7.35 (t, J=6.4 Hz, 1H), 6.97 (s, 1H), 6.89 (dd, J=1.8, 8.4 Hz, 1H), 5.04 (dd, J=5.4, 13.2 Hz, 1H), 4.01 (dd, J=2.3, 6.8 Hz, 2H), 2.93-2.82 (m, 1H), 2.64-2.53 (m, 2H), 2.06-2.00 (m, 1H), 1.43 (s, 9H).

Procedure for preparation of compound 643-4. To a solution of compound 643-3 (300 mg, 774.42 μmol, 1 eq) in DCM (0.5 mL) was added TFA (0.5 mL) at 0° C. The mixture was stirred at 25° C. for 8 h. The reaction was monitored by LCMS, LCMS showed compound 643-3 was consumed and the desired mass was detected. The mixture was filtered and concentrated to give compound 643-4 (250 mg, crude) as yellow solid. LCMS: t_{R}=0.270 min, MS (ESI+) m/z=331.9[M+1]⁺.

Procedure for preparation of compound MS-6-043. To a solution of compound 643-4 (120 mg, 362.23 μmol, 1 eq) in DMF (1 mL) was added compound 624-5 (156.67 mg, 362.23 μmol, 1 eq), HATU (206.60 mg, 543.35 μmol, 1.5 eq), DIEA (140.45 mg, 1.09 mmol, 189.28 uL, 3 eq), The mixture was stirred at 25° C. for 1 h. The reaction was monitored by LCMS, LCMS showed compound 643-4 was consumed and the desired mass was detected. The mixture was filtered and purified by prep-HPLC (36%-66% ACN in water (0.225% FA), 9 min) to give MS-6-043 (37.92 mg, 48.81 μmol, 13.48% yield, 96% purity) as yellow solid. LCMS: t_{R}=0.440 min, MS (ESI+) m/z=746.3[M+1]⁺. ¹H NMR (400 MHz, DMSO-d₆) δ=11.04 (s, 1H), 8.91-8.52 (m, 2H), 8.16-7.90 (m, 2H), 7.55-7.39 (m, 1H), 7.25-6.82 (m, 3H), 6.63 (d, J=4.8 Hz, 1H), 5.14 (s, 1H), 5.07 (s, 1H), 5.00 (ddd, J=5.4, 12.8, 15.6 Hz, 1H), 4.80 (s, 1H), 4.75-4.55 (m, 2H), 4.49 (q, J=6.8 Hz, 1H), 4.32-4.06 (m, 3H), 3.64-3.43 (m, 2H), 3.20 (s, 2H), 2.94 (s, 1H), 2.92-2.82 (m, 1H), 2.63-2.52 (m, 2H), 2.14-1.87 (m, 4H), 1.74-1.61 (m, 1H), 1.53-1.35 (m, 3H), 1.21-1.05 (m, 3H).

### Example 64

### Synthesis of MS-6-044

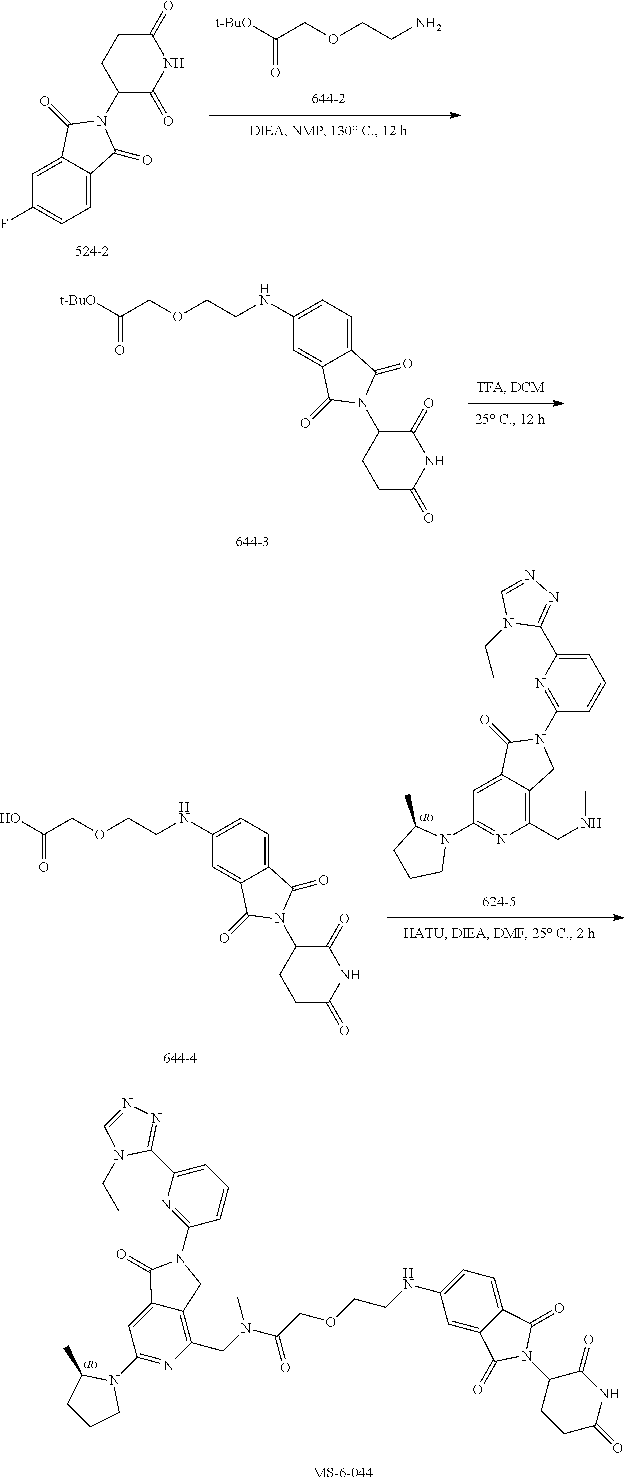

The procedure for preparing compound 624-5 is described in the synthesis of MS-6-027.

Procedure for preparation of compound 644-3. To a solution of t compound 524-2 (380.62 mg, 2.17 mmol, 1.2 eq) and compound 644-2 (500 mg, 1.81 mmol, 1 eq) in NMP (3 mL) was added DIEA (701.85 mg, 5.43 mmol, 945.89 μL, 3 eq). The mixture was stirred at 130° C. for 12 h. The reaction mixture was extracted with EtOAc (3 mL×3). The combined organic layers were dried over Na₂SO₄, filtered and concentrated under reduced pressure to give a residue, which was purified by prep-HPLC (30%-60% ACN in water (0.225% FA), 10 min) and lyophilized to give compound 644-3 (39 mg, 86.60 μmol, 4.78% yield) as a yellow solid. LCMS: t_{R}=0.401 min, MS (ESI+) m/z=432.17 [M+1]⁺.

Procedure for preparation of compound 644-4. To a solution of compound 644-3 (100 mg, 231.78 mol, 1 eq) in DCM (0.5 mL) was added TFA (0.5 mL) at 0° C. The mixture was stirred at 25° C. for 12 h. The reaction mixture was concentrated under reduced pressure to give a residue. The residue was purified by column chromatography (SiO₂, DCM/MeOH=100/0 to 10/1) to give compound 644-4 (68 mg, 181.17 μmol, 78.16% yield) as a green solid. LCMS: t_{R}=0.292 min, MS (ESI+) m/z=375.9 [M+1]⁺.

Procedure for preparation of MS-6-044. To a solution of compound 644-4 (68 mg, 181.17 μmol, 1 eq) and compound 624-5 (117.54 mg, 271.75 μmol, 1.5 eq) in DMF (1 mL) was added DIEA (117.08 mg, 905.85 μmol, 157.79 μL, 5 eq) and HATU (103.33 mg, 271.76 μmol, 1.5 eq). The mixture was stirred at 25° C. for 2 h. The residue was purified by prep-HPLC (36%-66% ACN in water (0.225% FA), 15 min) and lyophilized to give MS-6-044 (21.17 mg, 25.38 μmol, 14.01% yield) as a yellow solid. LCMS: t_{R}=1.465 min, MS (ESI+) m/z=790.3 [M+1]⁺. ¹H NMR (400 MHz, DMSO-d₆): δ=11.05 (s, 1H), 8.82 (s, 1H), 8.61 (t, J=6.8 Hz, 1H), 8.08 (dt, J=8.0, 2.4 Hz, 1H), 8.03-7.96 (m, 1H), 7.48 (dd, J=10.4, 8.0 Hz, 1H), 6.99-6.86 (m, 1H), 6.61 (d, J=4.4 Hz, 1H), 5.12-4.98 (m, 3H), 4.71-4.57 (m, 4H), 4.29 (s, 2H), 4.24-4.10 (m, 1H), 3.70-3.62 (m, 2H), 3.52 (s, 2H), 3.38-3.21 (m, 4H), 3.08-2.81 (m, 4H), 2.57 (d, J=18.0 Hz, 2H), 2.00 (d, J=15.6 Hz, 4H), 1.68 (s, 1H), 1.48 (td, J=10.4, 7.0 Hz, 3H), 1.15 (dd, J=18.6, 6.0 Hz, 3H).

### Example 65

### Synthesis of MS-6-045

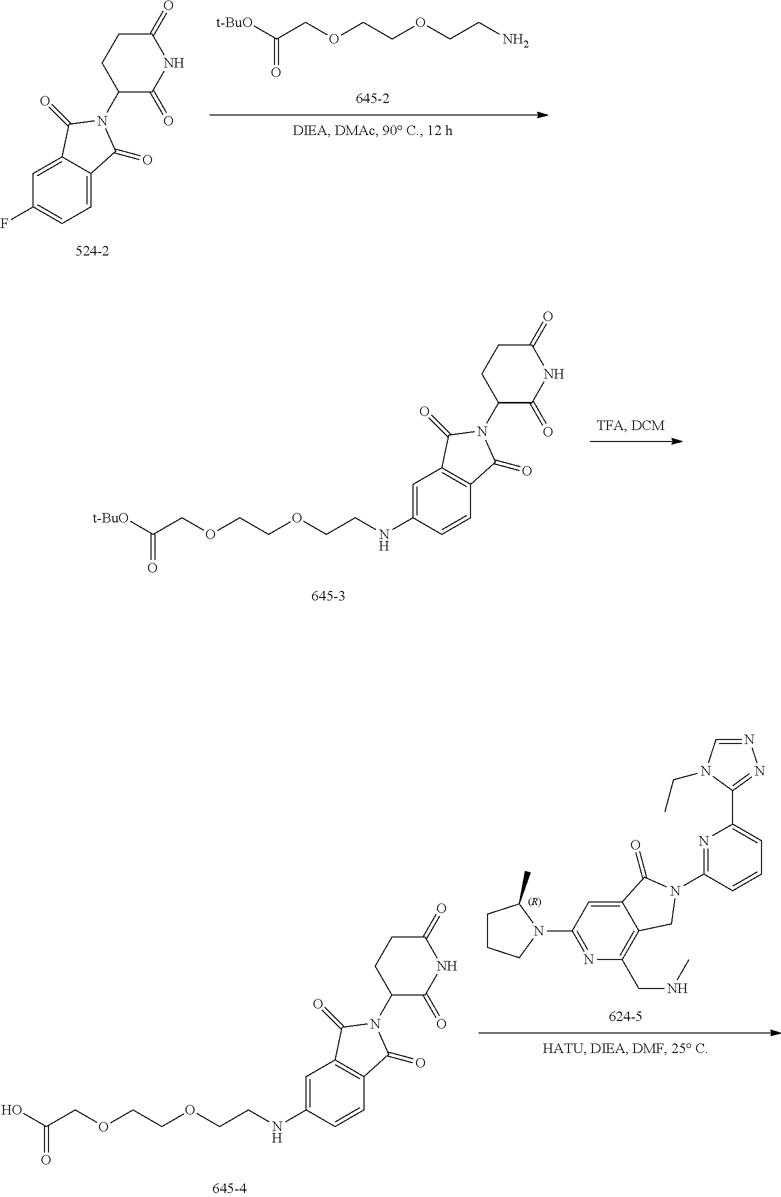

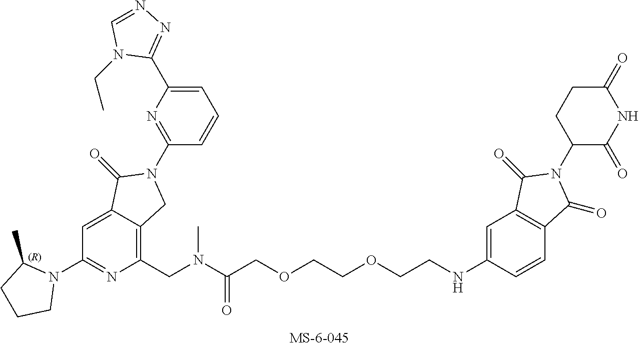

The procedure for preparing compound 624-5 is described in the synthesis of MS-6-027. Procedure for preparation of compound 645-3. To a solution of compound 524-2 in DMAc (5 mL) was added DIEA (1.47 g, 11.40 mmol, 1.99 mL, 5 eq). The mixture was stirred at 90° C. for 12 h. The reaction was monitored by LCMS, LCMS showed compound 645-1 was consumed and the desired mass was detected. The reaction mixture was concentrated under reduced pressure to remove solvent to give a residue, which was purified by prep-HPLC (30%-60% ACN in water (0.225% FA), 10 min). The result solution was lyophilized to give compound 645-3 (160 mg, 336.49 μmol, 14.76% yield) as green gum. LCMS: t_{R}=0.403 min, MS (ESI+) m/z=420.1[M+1]⁺. ¹H NMR (400 MHz, DMSO-d₆): δ=11.24-10.86 (m, 1H), 7.56 (d, J=8.0 Hz, 1H), 7.15 (t, J=5.4 Hz, 1H), 7.01 (d, J=1.8 Hz, 1H), 6.90 (dd, J=2.0, 8.8 Hz, 1H), 5.03 (dd, J=5.4, 12.8 Hz, 1H), 4.03-3.94 (m, 2H), 3.66-3.49 (m, 7H), 3.41-3.35 (m, 2H), 2.96-2.79 (m, 1H), 2.64-2.53 (m, 1H), 2.05-1.95 (m, 1H), 1.41 (s, 9H).

Procedure for preparation of compound 645-4. To a solution of compound 645-3 (150 mg, 315.46 μmol, 1 eq) in DCM (1 mL) was added TFA (1.54 g, 13.51 mmol, 1 mL, 42.81 eq). The mixture was stirred at 25° C. for 2 h. The reaction was monitored by LCMS, LCMS showed compound 645-3 was consumed and the desired mass was detected. The reaction mixture was concentrated under reduced pressure to remove solvents to give compound 645-4 (200 mg, crude, TFA) as brown liquid. LCMS: t_{R}=0.305 min, MS (ESI+) m/z=420.1[M+1]⁺.

Procedure for preparation of compound MS-6-045. To a solution of compound 645-4 (150 mg, 315.46 μmol, 1 eq) and compound 624-5 (154.06 mg, 356.20 μmol, 1 eq) in DMF (2.5 mL) was added HATU (203.16 mg, 534.30 μmol, 1.5 eq) and DIEA (184.15 mg, 1.42 mmol, 248.17 uL, 4 eq). The mixture was stirred at 25° C. for 2 h. The reaction was monitored by LCMS, LCMS showed compound 645-4 was consumed and the desired mass was detected. The reaction mixture was concentrated under reduced pressure to remove solvent, which was purified by prep-HPLC (31%-61% ACN in water (0.225% FA), 10 min). The result solution was lyophilized to give compound MS-6-045 (14.32 mg, 16.27 μmol, 4.57% yield, HCOOH) as yellow solid. LCMS: t_{R}=1.435 min, MS (ESI+) m/z=834.4 [M+1]⁺. ¹H NMR (400 MHz, DMSO-d₆): δ=11.05 (s, 1H), 8.74 (d, J=6.4 Hz, 1H), 8.62-8.58 (m, 1H), 8.41 (s, 1H), 8.10-8.05 (m, 1H), 7.99 (d, J=7.6 Hz, 1H), 7.48 (d, J=8.4 Hz, 1H), 7.10 (dd, J=5.2, 3.6 Hz, 1H), 6.95 (s, 1H), 6.82 (d, J=8.4 Hz, 1H), 6.62 (d, J=4.0 Hz, 1H), 5.10-5.06 (m, 2H), 5.00 (dd, J=5.6, 12.8 Hz, 1H), 4.58-4.58 (m, 1H), 4.69-4.55 (m, 4H), 4.33-4.23 (m, 2H), 4.22-4.15 (m, 1H), 3.56 (dd, J=17.6, 4.4 Hz, 8H), 3.05 (s, 2H), 2.93-2.80 (m, 3H), 2.58 (s, 2H), 2.06-1.96 (m, 4H), 1.69-1.64 (m, 1H), 1.51-1.45 (m, 3H), 1.19-1.12 (m, 3H).

### Example 66

### Synthesis of MS-6-046

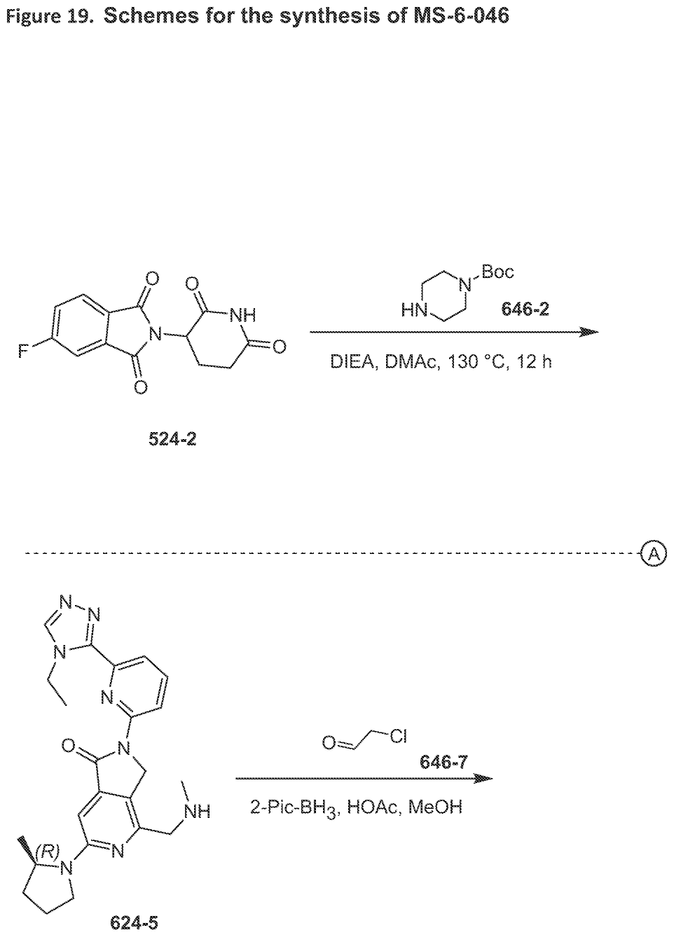

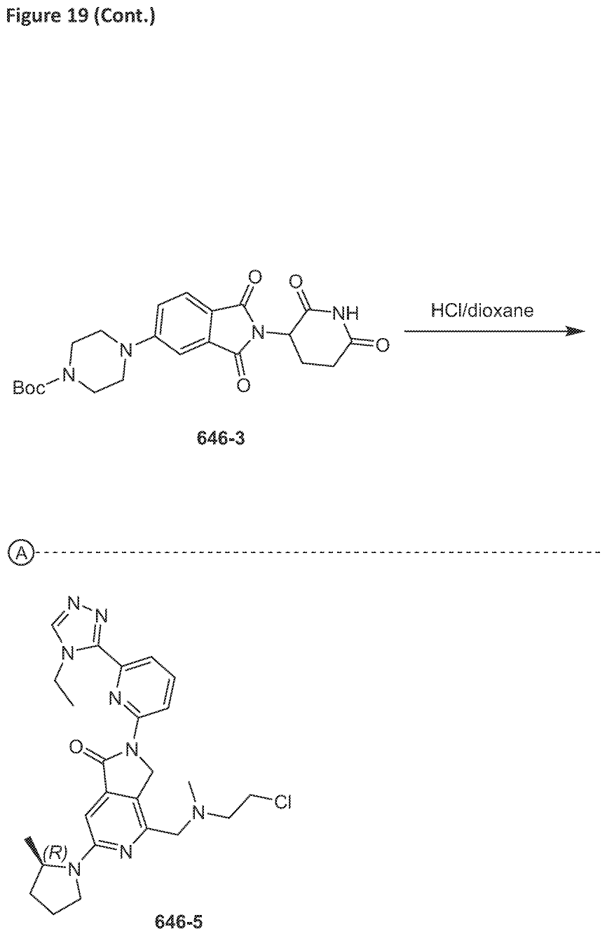

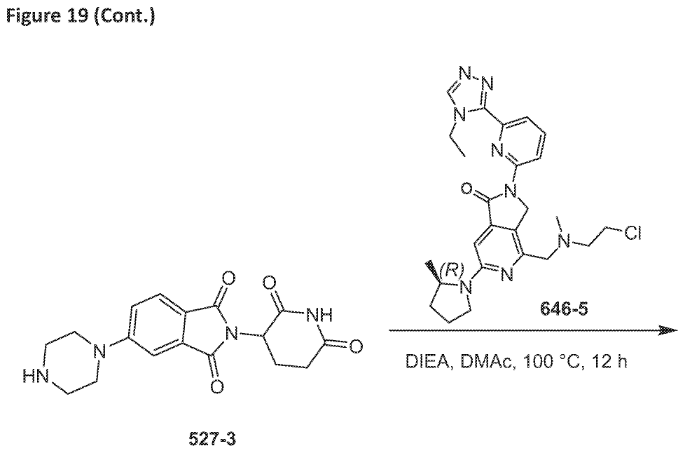

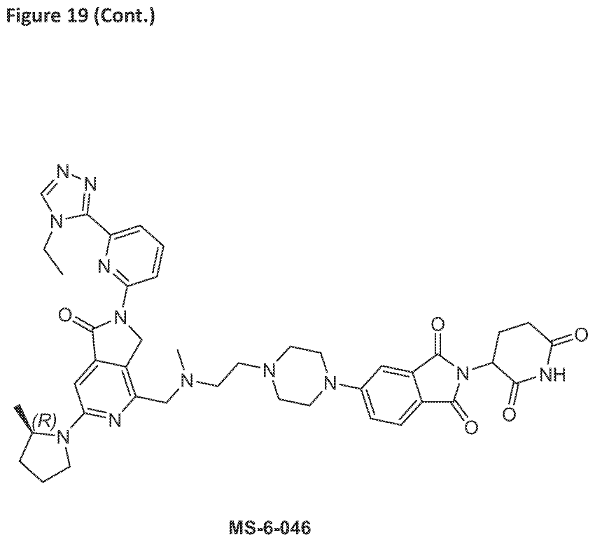

The reaction scheme for the synthesis of MS-6-046 is shown in FIG. 19.

The procedure for preparing compound 624-5 is described in the synthesis of MS-6-027.

Procedure for preparation of compound 646-3. To a solution of compound 646-2 (20.23 g, 108.61 mmol, 1.5 eq) in NMP (500 mL) was added DIEA (9.36 g, 72.41 mmol, 12.61 mL, 1 eq), compound 524-2 (20 g, 72.41 mmol, 1 eq). The mixture was stirred at 130° C. for 24 h under an N₂ atmosphere. The reaction was monitored by LCMS, LCMS showed compound 524-2 was consumed and the desired mass was detected. After cooling, the mixture was poured into water (1.5 L), the suspension was filtered and the filter cake was dried in vacuo to give compound 646-3 (32 g, 72.32 mmol, 99.88% yield) as green solid. LCMS: t_{R}=0.445 min, MS (ESI+) m/z=387.0[M-56+1]⁺. ¹H NMR (400 MHz, DMSO-d₆) δ=11.08 (s, 1H), 7.69 (d, J=8.4 Hz, 1H), 7.34 (d, J=2.4 Hz, 1H), 7.24 (dd, J=8.4, 2.4 Hz, 1H), 5.07 (dd, J=12.8, 5.4 Hz, 1H), 3.46 (s, 8H), 2.96-2.79 (m, 1H), 2.62-2.52 (m, 2H), 2.07-1.96 (m, 1H), 1.42 (s, 9H).

Procedure for preparation of compound 527-3. To a solution of compound 646-3 (5 g, 11.30 mmol, 1 eq) in DCM (25 mL) was added HCl/dioxane (4 M, 14.13 mL, 5 eq). The mixture was stirred at 25° C. for 1 h. The reaction was monitored by LCMS, LCMS showed compound 646-3 was consumed and the desired mass was detected. The mixture was filtered to give compound 527-3 (4.2 g, crude, HCl) as brown solid. LCMS: t_{R}=0.211 min, MS (ESI+) m/z=343.2[M+1]⁺. ¹H NMR (400 MHz, DMSO-d₆) δ=11.08 (s, 1H), 9.63 (br s, 2H), 7.73 (d, J=8.4 Hz, 1H), 7.44 (d, J=2.4 Hz, 1H), 7.32 (dd, J=8.4, 2.4 Hz, 1H), 5.08 (dd, J=12.8, 5.2 Hz, 1H), 3.80-3.63 (m, 4H), 3.25-3.12 (m, 4H), 2.95-2.81 (m, 1H), 2.63-2.52 (m, 2H), 2.06-1.99 (m, 1H).

Procedure for preparation of compound MS-6-046. A vial charged a stir bar was added compound 646-4 (105.02 mg, 277.24 μmol, 1.5 eq, HCl), compound 646-5 (100 mg, 184.83 μmol, 1 eq, FA). Then NMP (1 mL) and DIEA (119.44 mg, 924.14 μmol, 160.97 uL, 5 eq) were added. The mixture was stirred at 100° C. for 12 h. The reaction was monitored by LCMS, LCMS showed compound 646-4 was consumed and the desired mass was detected. The mixture was filtered and purified by prep-HPLC (15%-45% ACN in water (0.225% FA), 8 min) to give MS-6-046 (39.39 mg, 46.04 μmol, 24.91% yield, 99% purity, FA) as yellow solid. LCMS: t_{R}=0.380 min, MS (ESI+) m/z=801.3[M+1]⁺. ¹H NMR (400 MHz, DMSO-d₆) δ=11.09 (s, 1H), 8.75 (s, 1H), 8.52 (dd, J=7.2, 1.8 Hz, 1H), 7.99-7.88 (m, 2H), 7.62 (d, J=8.4 Hz, 1H), 7.15 (s, 1H), 7.07 (dd, J=8.6, 1.8 Hz, 1H), 6.59 (s, 1H), 5.26 (s, 2H), 5.07 (dd, J=12.8, 5.4 Hz, 1H), 4.67 (q, J=7.2 Hz, 2H), 4.26-4.15 (m, 1H), 3.78-3.69 (m, 2H), 3.53 (br dd, J=9.2, 7.6 Hz, 2H), 3.17 (s, 6H), 2.94-2.83 (m, 1H), 2.62-2.53 (m, 2H), 2.47-2.35 (m, 6H), 2.32 (s, 3H), 2.11-1.93 (m, 4H), 1.69 (s, 1H), 1.58-1.47 (m, 3H), 1.19 (d, J=6.4 Hz, 3H).

### Example 67

### Synthesis of MS-6-047

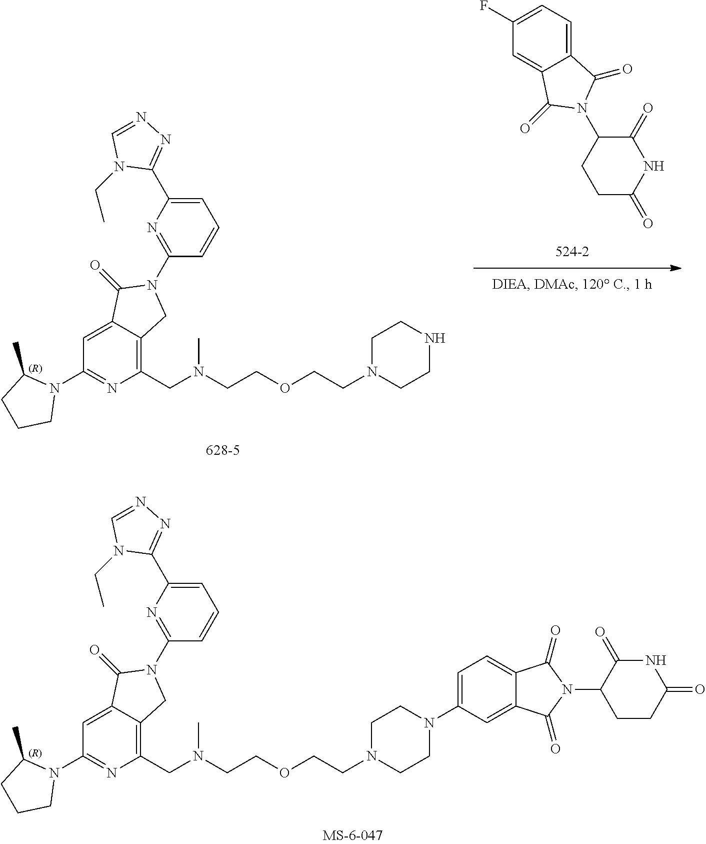

The procedure for preparing compound 628-5 is described in the synthesis of MS-6-028.

Procedure for preparation of MS-6-047. To a solution of compound 628-5 (100 mg, 103.61 μmol, 61% purity, 1 eq) and compound 524-2 (28.62 mg, 103.61 μmol, 1 eq) in DMAc (1 mL) was added DIEA (13.39 mg, 103.61 μmol, 18.05 uL, 1 eq) and the mixture was stirred at 120° C. for 1 h. The residue was purified by prep-HPLC (7%-37% ACN in water (0.225% FA), 10 min) and lyophilized to give MS-6-047 (21.52 mg, 24.08 μmol, 23.24% yield, 99.7% purity, FA) as a yellow solid. LCMS: t_{R}=1.402 min, MS (ESI+) m/z=845.3 [M+1]⁺. ¹H NMR (400 MHz, DMSO-d₆) δ=11.09 (s, 1H), 8.75 (s, 1H), 8.61 (d, J=8.4 Hz, 1H), 8.21 (s, 1H), 8.10-8.03 (m, 1H), 8.02-7.97 (m, 1H), 7.66 (d, J=8.4 Hz, 1H), 7.23 (s, 1H), 7.15 (d, J=7.2 Hz, 1H), 6.58 (s, 1H), 5.22 (s, 2H), 5.08 (dd, J=12.8, 5.6 Hz, 1H), 4.68 (q, J=7.2 Hz, 2H), 4.23-4.14 (m, 1H), 3.76 (s, 2H), 3.51 (d, J=5.2 Hz, 2H), 3.40 (s, 2H), 3.30 (d, J=9.2 Hz, 4H), 3.23 (s, 4H), 2.93-2.86 (m, 1H), 2.61 (d, J=2.4 Hz, 2H), 2.56 (d, J=5.4 Hz, 2H), 2.31 (s, 7H), 2.08-1.92 (m, 4H), 1.67 (s, 1H), 1.51 (t, J=7.2 Hz, 3H), 1.18 (d, J=6.4 Hz, 3H).

### Example 68

### Synthesis of MS-6-048

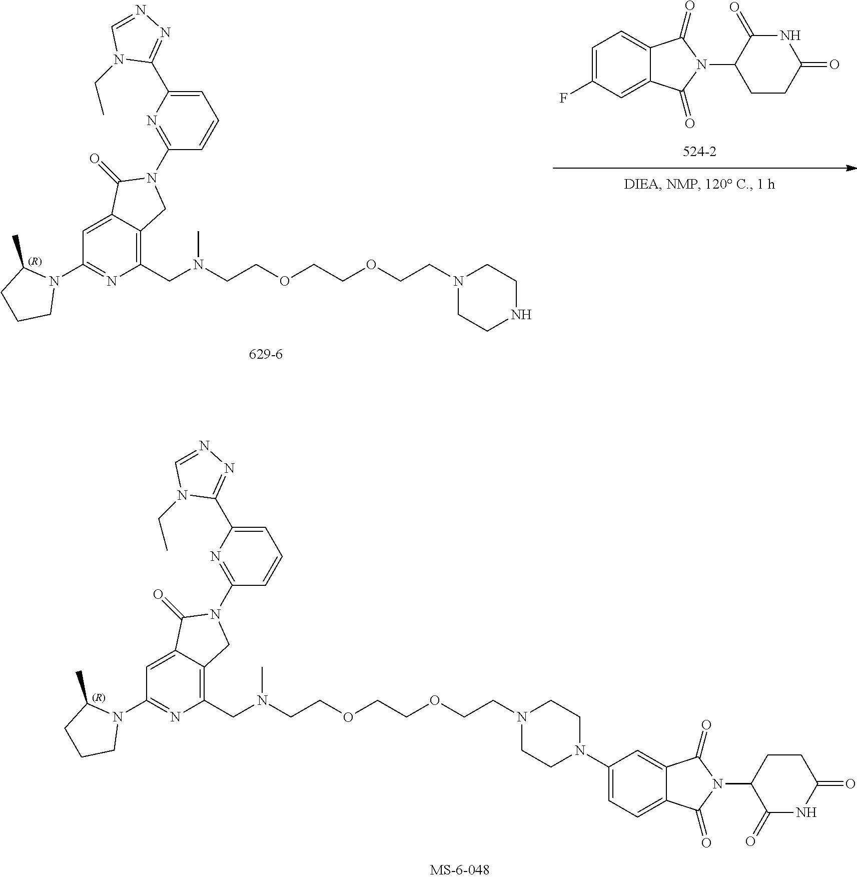

The procedure for preparing compound 629-6 is described in the synthesis of MS-6-029.

Procedure for preparation of compound MS-6-048. To a solution of compound 629-6 (55 mg, 86.92 μmol, 1 eq) in NMP (0.5 mL) was added compound 524-2 (28.81 mg, 104.30 μmol, 1.2 eq) and DIEA (33.70 mg, 260.75 μmol, 45.42 uL, 3 eq). The vial was heated at 120° C. for 12 h. The reaction was monitored by LCMS, LCMS showed compound 648-1 was consumed and the desired mass was detected. The mixture was filtered and purified by prep-HPLC (38%-68% ACN in water, 8 min) to give MS-6-048 (10 mg, 10.45 μmol, 12.02% yield, 92.9% purity) as yellow solid. LCMS: t_{R}=0.344 min, MS (ESI+) m/z=889.4[M+1]⁺. ¹H NMR (400 MHz, DMSO-d₆) δ=11.07 (s, 1H), 8.74 (s, 1H), 8.62 (d, J=8.0 Hz, 1H), 8.07 (t, J=7.6 Hz, 1H), 8.01 (d, J=7.2 Hz, 1H), 7.64 (d, J=8.6 Hz, 1H), 7.30-7.24 (m, 1H), 7.17 (dd, J=2.0, 8.4 Hz, 1H), 6.56 (s, 1H), 5.19 (s, 2H), 5.07 (dd, J=13.2, 5.6 Hz, 1H), 4.67 (q, J=6.8 Hz, 2H), 4.17 (t, J=5.6 Hz, 1H), 3.73 (s, 2H), 3.54-3.49 (m, 2H), 3.41-3.36 (m, 2H), 3.25 (br s, 10H), 2.95-2.81 (m, 1H), 2.68-2.52 (m, 4H), 2.44-2.38 (m, 4H), 2.32 (t, J=5.6 Hz, 2H), 2.29 (s, 3H), 2.07-1.91 (m, 4H), 1.67 (s, 1H), 1.49 (t, J=7.2 Hz, 3H), 1.17 (d, J=6.0 Hz, 3H).

### Example 69

### Synthesis of MS-6-049

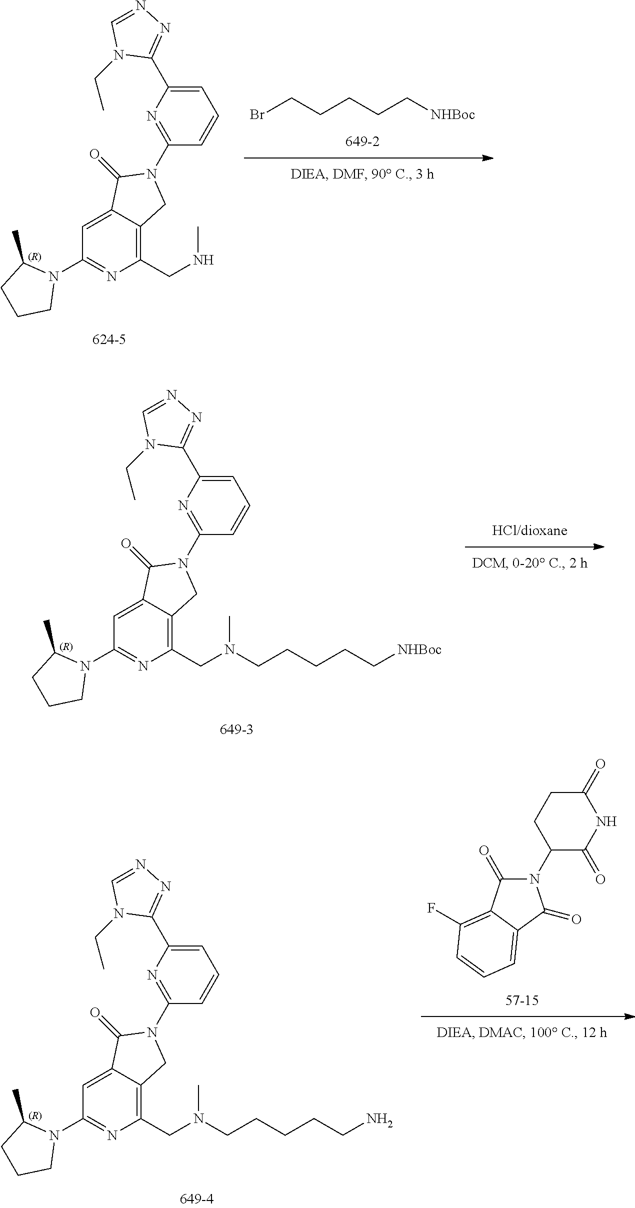

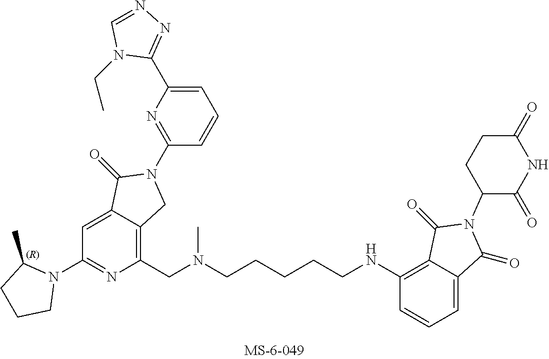

The procedure for preparing compound 624-5 is described in the synthesis of MS-6-027.

Procedure for preparation of compound 649-3. To a solution of compound 624-5 (300 mg, 693.61 μmol, 1 eq) and compound 649-2 (276.93 mg, 1.04 mmol, 1.5 eq) in DMF (3 mL) was added DIEA (179.29 mg, 1.39 mmol, 241.63 uL, 2 eq). The mixture was stirred at 90° C. for 3 h. The reaction was monitored by LCMS. LCMS showed compound 649-1 was consumed and the desired mass was detected. The reaction mixture was filtered and the filtrate was concentrated under the reduced pressure to give the product, which was purified by prep-HPLC (16%-46% ACN in water (0.225% FA), 10 min). The result solution was lyophilized to give compound 649-3 (186 mg, 292.07 μmol, 42.11% yield, 97.01% purity) as a yellow solid. LCMS: t_{R}=0.417 min, MS (ESI+) m/z=618.4 [M+1]⁺. ¹H NMR (400 MHz, DMSO-d6) δ=8.75 (s, 1H), 8.62 (d, J=8.4 Hz, 1H), 8.16-8.07 (m, 1H), 8.00 (d, J=7.2 Hz, 1H), 6.77 (s, 1H), 5.16 (s, 2H), 4.63 (q, J=7.0 Hz, 2H), 4.54-4.38 (m, 1H), 4.27 (s, 2H), 3.59 (s, 1H), 3.36-3.33 (m, 4H), 3.00-2.78 (m, 4H), 2.67 (s, 2H), 2.16-1.86 (m, 4H), 1.72 (s, 2H), 1.48 (t, J=7.2 Hz, 3H), 1.34 (s, 10H), 1.25 (s, 1H), 1.19 (d, J=6.4 Hz, 4H).

Procedure for preparation of compound 649-2. To a solution of compound 649-3 (180 mg, 291.36 μmol, 1 eq) in DCM (3 mL) was added HCl/dioxane (4 M, 218.52 uL, 3 eq). The mixture was stirred at 25° C. for 2 h. The reaction was monitored by LCMS. LCMS showed compound 649-3 was consumed and the desired mass was detected. The reaction mixture was filtered and the filtrate was concentrated under the reduced pressure to give the product, which was concentrated in vacuum and the reaction mixture was added water and NaHCO₃ (aq.) until pH around 8, then extracted with EtOAc (10 mL×3). The combined organic phase was washed with brine (10 mL×3), dried over anhydrous Na₂SO₄, filtered and concentrated in vacuo to give compound 649-4 (124 mg, 234.31 μmol, 80.42% yield, 97.82% purity) as a yellow solid. LCMS: t_{R}=0.304 min, MS (ESI+) m/z=518.3 [M+1]⁺.

Procedure for preparation of compound MS-6-049. To a solution of compound 649-4 (80 mg, 154.54 μmol, 1 eq) and compound 57-15 (42.69 mg, 154.54 μmol, 1 eq) in DMAc (1 mL) was added DIEA (39.95 mg, 309.08 μmol, 53.84 uL, 2 eq). The mixture was stirred at 100° C. for 12 h. The reaction was monitored by LCMS, LCMS showed compound 649-4 was consumed. Several new peaks were shown on LC-MS and 26% of desired compound was detected. The reaction mixture was concentrated under reduced pressure to remove solvent, which was purified by prep-HPLC (20%-50% ACN in water (0.225% FA), 10 min). The result solution was lyophilized to give compound MS-6-049 (8.95 mg, 11.27 μmol, 7.30% yield, 97.48% purity) was obtained as yellow solid. LCMS: t_{R}=1.430 min, MS (ESI+) m/z=774.4[M+1]⁺. ¹H NMR (400 MHz, DMSO-d₆): δ=11.07 (s, 1H), 8.70 (s, 1H), 8.59 (d, J=8.4 Hz, 1H), 8.08-8.03 (m, 1H), 8.00-7.96 (m, 1H), 7.50-7.44 (m, 1H), 6.98-6.92 (m, 2H), 6.58 (s, 1H), 6.41 (t, J=5.6 Hz, 1H), 5.12 (s, 2H), 5.01 (dd, J=5.2, 12.8 Hz, 1H), 4.62 (q, J=6.8 Hz, 2H), 4.23-4.16 (m, 1H), 3.66 (d, J=3.2 Hz, 2H), 3.55-3.49 (m, 1H), 3.22-3.12 (m, 3H), 2.89-2.82 (m, 1H), 2.59-2.55 (m, 2H), 2.40-2.36 (m, 2H), 2.22 (s, 3H), 2.09-1.94 (m, 5H), 1.70-1.66 (m, 1H), 1.53-1.46 (m, 6H), 1.34-1.28 (m, 2H), 1.18 (d, J=6.4 Hz, 3H).

### Example 70

### Synthesis of MS-6-050

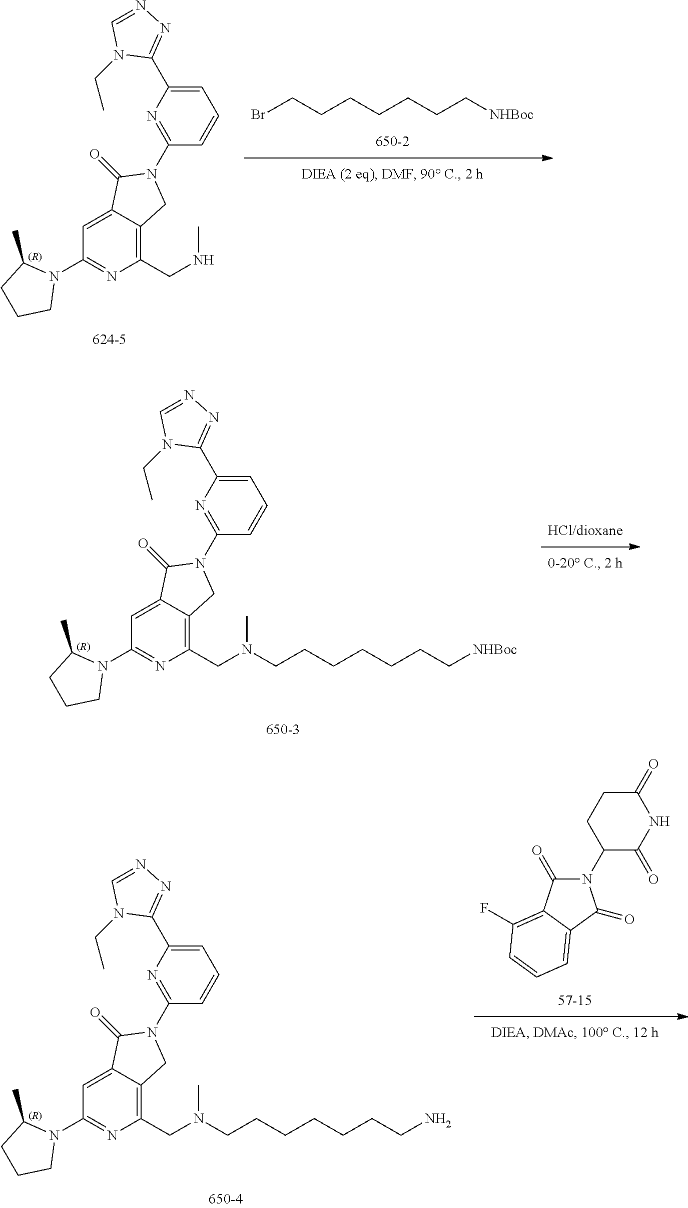

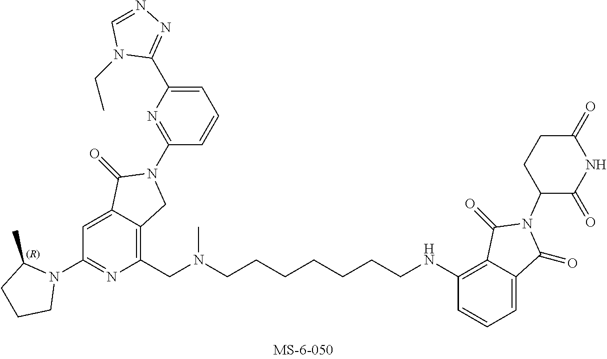

The procedure for preparing compound 624-5 is described in the synthesis of MS-6-027.

Procedure for preparation of compound 650-3. To a solution of compound 650-1 (600 mg, 1.39 mmol, 1 eq) and compound 650-2 (408.16 mg, 1.39 mmol, 1 eq) in DMF (4 mL) was added DIEA (358.58 mg, 2.77 mmol, 483.26 uL, 2 eq). The mixture was stirred at 90° C. for 2 h. The reaction was monitored by LCMS. LCMS showed compound 650-1 was consumed and the desired mass was detected. The reaction mixture was concentrated under reduced pressure to remove solvent, which was purified by prep-HPLC (13%-43% ACN in water (0.225% FA), 10 min). The result solution was lyophilized to give compound 650-3 (140 mg, 216.77 μmol, 15.63% yield) as brown solid. LCMS: t_{R}=0.448 min, MS (ESI+) m/z=646.4[M+1]⁺. ¹H NMR (400 MHz, DMSO-d₆): δ=8.74 (s, 1H), 8.58 (d, J=8.0 Hz, 1H), 8.08-7.94 (m, 2H), 6.68 (t, J=5.2 Hz, 1H), 6.59-6.50 (m, 1H), 5.06 (s, 2H), 4.62 (q, J=7.0 Hz, 2H), 4.23-4.10 (m, 1H), 3.71-3.57 (m, 2H), 3.49 (t, J=7.4 Hz, 1H), 3.33-3.22 (m, 1H), 2.80 (q, J=6.4 Hz, 2H), 2.36 (t, J=7.2 Hz, 2H), 2.21 (s, 3H), 2.04 (d, J=5.2 Hz, 2H), 1.93 (d, J=2.6 Hz, 1H), 1.70-1.64 (m, 1H), 1.49-1.41 (m, 5H), 1.33 (s, 9H), 1.28-1.23 (m, 2H), 1.17 (d, J=6.0 Hz, 9H).

Procedure for preparation of compound 650-4. To a solution of compound 650-3 (130 mg, 201.29 μmol, 1 eq) in DCM (2 mL) was added HCl/dioxane (4 M, 251.61 uL, 5 eq) at 0° C. The mixture was stirred at 25° C. for 2 h. The reaction was monitored by LCMS, LCMS showed compound 650-3 was consumed and the desired mass was detected. The reaction mixture was concentrated under reduced pressure to remove solvent. Compound 650-4 (100 mg, 171.77 μmol, 85.33% yield, HCl) was brown solid. LCMS: t_{R}=0.332 min, MS (ESI+) m/z=546.5[M+1]⁺.

Procedure for preparation of compound MS-6-050. To a solution of compound 650-4 (90 mg, 154.59 μmol, 1 eq, HCl) and 2-(2,6-dioxo-3-piperidyl)-4-fluoro-isoindoline-1,3-dione (45.54 mg, 164.87 μmol, 1.07 eq) in DMAC (1 mL) was added DIEA (42.66 mg, 330.08 μmol, 57.49 uL, 2.14 eq). The mixture was stirred at 100° C. for 12 h. The reaction was monitored by LCMS, LCMS showed compound 650-4 was consumed and the desired mass was detected. The reaction mixture was concentrated under reduced pressure to remove solvent, which was purified by prep-HPLC (column: Phenomenex C18 150×25 mm×10 um; mobile phase: [water (NH₄HCO₃)-ACN]; B %: 60%-90%, 8 min), the result solution was lyophilized to give compound MS-6-050 (10.95 mg, 13.65 μmol, 8.83% yield) as yellow solid. LCMS: t_{R}=1.506 min, MS (ESI+) m/z=802.6[M+1]⁺. ¹H NMR (400 MHz, DMSO-d₆): δ=11.34-10.96 (m, 1H), 8.74 (s, 1H), 8.64-8.60 (m, 1H), 8.09-8.04 (m, 1H), 8.00-7.97 (m, 1H), 7.53 (dd, J=7.2, 8.4 Hz, 1H), 7.01-6.97 (m, 2H), 6.59 (s, 1H), 6.45-6.40 (m, 1H), 5.14 (s, 2H), 5.02 (dd, J=5.2, 12.4 Hz, 1H), 4.64 (q, J=7.2 Hz, 2H), 4.22-4.16 (m, 1H), 3.65 (d, J=4.0 Hz, 2H), 3.17-3.12 (m, 2H), 2.89-2.83 (m, 1H), 2.60 (d, J=4.4 Hz, 2H), 2.55 (s, 2H), 2.38-2.34 (m, 2H), 2.21 (s, 3H), 2.06-1.93 (m, 5H), 1.70-1.66 (m, 1H), 1.50-1.44 (m, 6H), 1.22-1.16 (m, 9H).

### Example 71

### Synthesis of MS-6-051

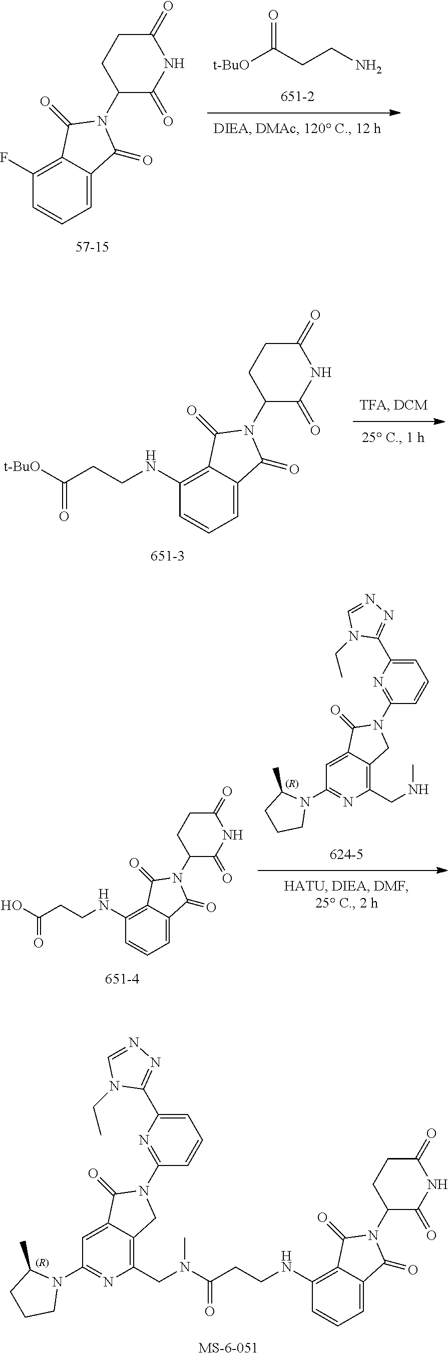

The procedure for preparing compound 624-5 is described in the synthesis of MS-6-027. Procedure for preparation of compound 651-3. To a solution of compound 651-2 (500.00 mg, 3.44 mmol, 1 eq) and compound 57-15 (1.43 g, 5.17 mmol, 1.5 eq) in DMAc (2 mL) was added DIEA (1.34 g, 10.33 mmol, 1.80 mL, 3 eq). The mixture was stirred at 120° C. for 12 h. The residue was purified by prep-TLC (SiO₂, Petroleum ether/Ethyl acetate=4/1, Rf=0.5) and eluted with ACN (50 mL) to give compound 651-3 (576 mg, 229.59 μmol, 6.67% yield) as a green solid. LCMS: t_{R}=0.457 min, MS (ESI+) m/z=402.16 [M+1]⁺. ¹H NMR (400 MHz, DMSO-d₆) δ=11.05 (s, 1H), 7.55 (t, J=7.2 Hz, 1H), 7.76-7.51 (m, 1H), 7.01 (d, J=7.2 Hz, 1H), 7.14-6.96 (m, 1H), 6.59 (t, J=6.0 Hz, 1H), 5.12 (dd, J=112.8, 5.2 Hz, OH), 5.02 (dd, J=12.8, 5.2 Hz, 1H), 3.49 (q, J=6.4 Hz, 1H), 3.30 (s, 5H), 2.93-2.76 (m, 1H), 2.55-2.44 (m, 5H), 1.98-1.94 (m, 1H), 1.94-1.93 (m, 1H), 1.35 (s, 5H), 1.13 (t, J=7.2 Hz, 1H).

Procedure for preparation of compound 651-4. To a solution of compound 651-3 (200 mg, 498.24 μmol, 1 eq) in DCM (2 mL) was added TFA (284.05 mg, 2.49 mmol, 184.45 uL, 5 eq). The mixture was stirred at 25° C. for 1 h. The reaction mixture was filtered and the filter cake was dyed under the reduced pressure to give compound 651-4 (204 mg, 590.78 μmol, crude product) as a yellow solid. LCMS: t_{R}=0.306 min, MS (ESI+) m/z=346.1 [M+1]⁺.

Procedure for preparation of MS-6-051. To a solution of d compound 651-4 (204 mg, 590.78 μmol, 1 eq) and d compound 624-5 (383.29 mg, 886.17 μmol, 1.5 eq) in DMAc (2 mL) was added HATU (336.95 mg, 886.17 μmol, 1.5 eq) and DIEA (381.77 mg, 2.95 mmol, 514.52 uL, 5 eq). The mixture was stirred at 25° C. for 2 h. The residue was purified by prep-HPLC (41%-71% ACN in water (0.225% FA), 10 min) and lyophilized to give MS-6-051 (98.78 mg, 129.25 μmol, 21.88% yield) as a yellow solid. LCMS: t_{R}=0.459 min, MS (ESI+) m/z=760.32 [M+1]⁺. ¹H NMR (400 MHz, DMSO-d₆) δ=11.08 (s, 1H), 8.75 (d, J=17.0 Hz, 1H), 8.60 (t, J=6.0 Hz, 1H), 8.11-8.03 (m, 1H), 7.99 (d, J=7.2 Hz, 1H), 7.49 (t, J=7.2 Hz, 1H), 7.12-7.00 (m, 1H), 6.96 (dd, J=6.8, 4.4 Hz, 1H), 6.83-6.68 (m, 1H), 6.59 (d, J=3.6 Hz, 1H), 5.09 (d, J=17.6 Hz, 2H), 5.04-4.90 (m, 1H), 4.74-4.55 (m, 4H), 4.20-4.00 (m, 1H), 3.60-3.39 (m, 4H), 3.14-2.90 (m, 3H), 2.88-2.68 (m, 3H), 2.62-2.51 (m, 2H), 2.03-1.83 (m, 4H), 1.66-1.55 (m, 1H), 1.49 (td, J=13.8, 7.2 Hz, 3H), 1.17-1.04 (m, 3H).

### Example 72

### Synthesis of MS-6-052

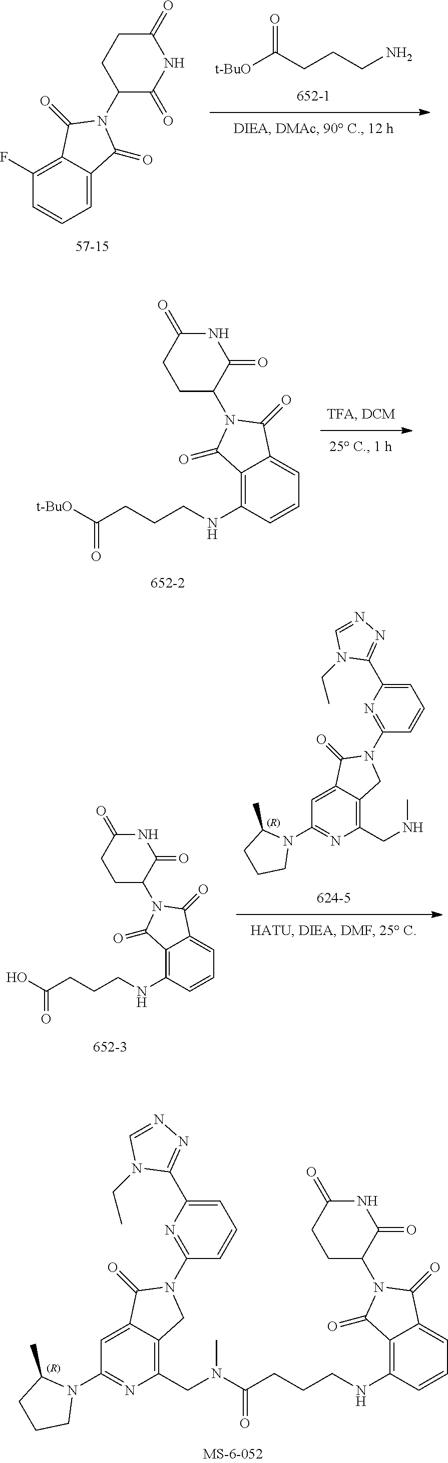

Procedure for preparation of compound 652-2. To a solution of compound 57-15 (1.30 g, 4.71 mmol, 1.5 eq) and compound 652-1 (500 mg, 3.14 mmol, 1 eq) in DMAc (2 mL) was added DIEA (1.22 g, 9.42 mmol, 1.64 mL, 3 eq). The mixture was stirred at 90° C. for 12 h. The residue was purified by column chromatography (SiO₂, Petroleum ether/Ethyl acetate=100/0 to 4/1) to give compound 652-2 (935 mg, 729.21 μmol, 23.22% yield) as a green solid. LCMS: t_{R}=0.449 min, MS (ESI+) m/z=416.17 [M+1]⁺. ¹H NMR (400 MHz, DMSO-d₆) δ=11.14 (s, 1H), 11.09 (s, 1H), 7.98-7.87 (m, 1H), 7.81-7.71 (m, 1H), 7.58 (t, J=7.8 Hz, 1H), 5.04 (dd, J=12.8, 5.2 Hz, 1H), 5.15 (dd, J=12.8, 5.2 Hz, 1H), 3.47-3.38 (m, 2H), 3.33-3.21 (m, 2H), 3.09-2.79 (m, 2H), 2.63-2.51 (m, 3H), 2.31-2.24 (m, 1H), 1.98 (s, 1H), 2.14-1.94 (m, 1H), 1.77 (m, 1H), 1.24-1.14 (m, 1H), 1.38 (s, 4H)

Procedure for preparation of compound 652-3. To a solution of compound 652-2 (400 mg, 962.84 μmol, 1 eq) in DCM (2 mL) was added TFA (548.93 mg, 4.81 mmol, 356.45 uL, 5 eq). The mixture was stirred at 25° C. for 1 h. The reaction mixture was filtered and the filtrate was concentrated under the reduced pressure to give compound 652-3 (150 mg, 153.16 μmol, 15.91% yield) as a green solid. LCMS: t_{R}=0.304 min, MS (ESI+) m/z=360.11 [M+1]⁺

Procedure for preparation of MS-6-052. To a solution of compound 652-3 (150 mg, 417.44 μmol, 1 eq) and compound 624-5 (270.83 mg, 626.16 μmol, 1.5 eq) in DMAc (2 mL) was added HATU (79.36 mg, 208.72 μmol, 0.5 eq) and DIEA (269.75 mg, 2.09 mmol, 363.54 uL, 5 eq). The mixture was stirred at 25° C. for 2 h. The residue was purified by prep-HPLC (42%-72% ACN in water (0.225% FA), 10 min) and lyophilized to give compound MS-6-052 (98.65 mg, 124.35 μmol, 29.79% yield) as a yellow solid. LCMS: t_{R}=0.463 min, MS (ESI+) m/z=774.34 [M+1]⁺. ¹H NMR (400 MHz, DMSO-d₆) δ=11.08 (s, 1H), 8.76 (d, J=12.0 Hz, 1H), 8.60 (dd, J=8.4, 3.6 Hz, 1H), 8.07 (dt, J=8.0, 4.0 Hz, 1H), 7.99 (d, J=7.6 Hz, 1H), 7.53-7.44 (m, 1H), 7.13-7.06 (m, 1H), 6.97 (t, J=6.4 Hz, 1H), 6.58 (s, 2H), 5.13-4.98 (m, 3H), 4.71-4.54 (m, 4H), 4.21-4.07 (m, 1H), 3.59-3.39 (m, 2H), 3.35-3.19 (m, 4H), 3.14-2.89 (m, 3H), 2.89-2.81 (m, 1H), 2.62-2.52 (m, 2H), 2.06-1.89 (m, 4H), 1.86-1.76 (m, 2H), 1.70-1.59 (m, 1H), 1.48 (td, J=9.6, 7.2 Hz, 3H), 1.13 (dd, J=17.6, 6.0 Hz, 3H) Example 73

### Synthesis of MS-6-053

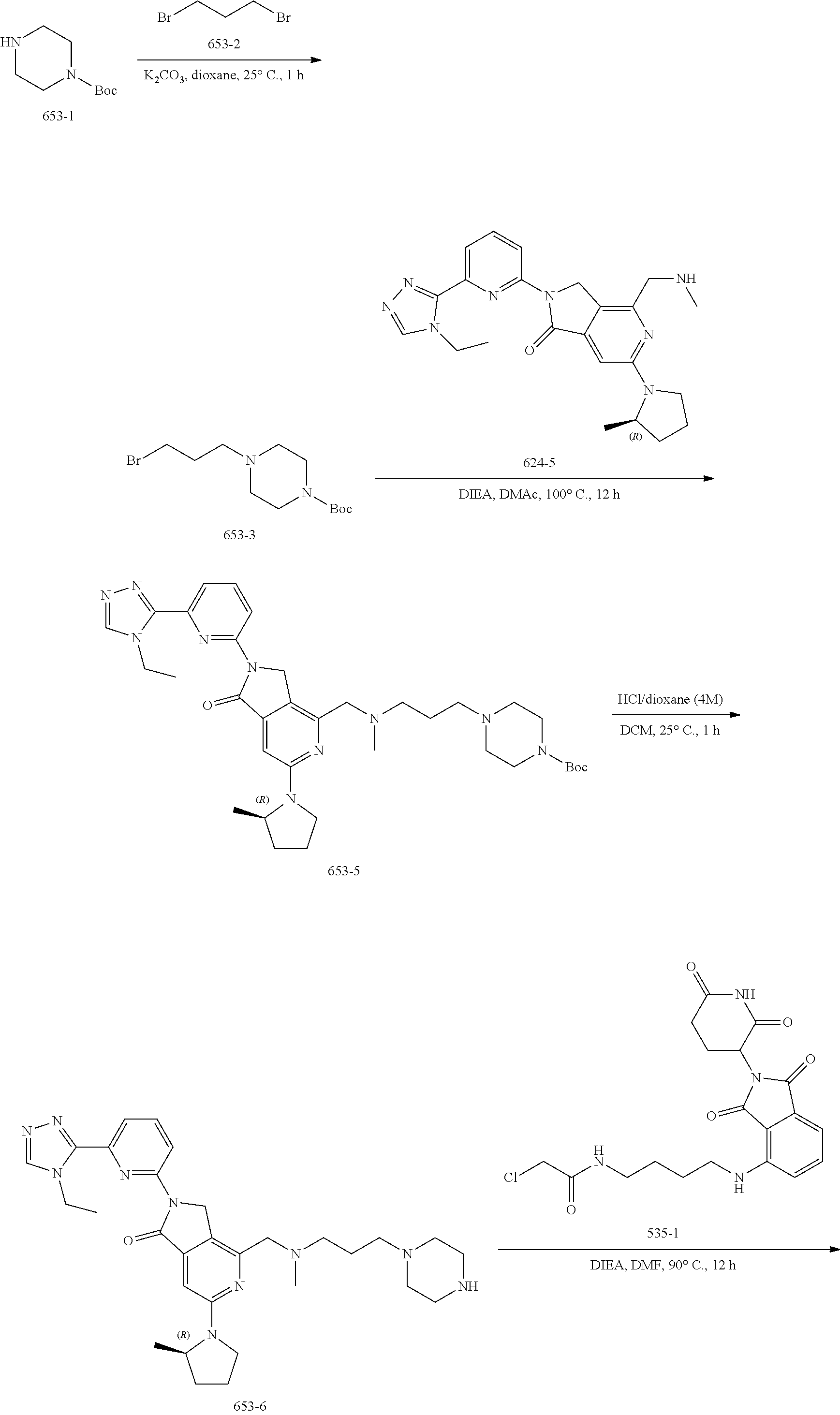

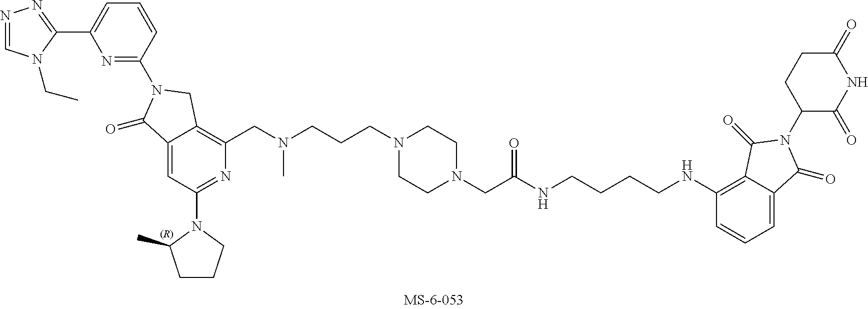

The procedure for preparing compound 624-5 is described in the synthesis of MS-6-027.

Procedure for preparation of compound 653-3. To a solution of compound 653-2 (108.40 g, 536.91 mmol, 54.75 mL, 10 eq) and compound 653-1 (10 g, 53.69 mmol, 1 eq) in dioxane (200 mL) was added K₂CO₃ (37.10 g, 268.46 mmol, 5 eq). The mixture was stirred at 25° C. for 12 h. The reaction mixture was diluted with H₂O (200 mL) and extracted with ethyl acetate (300 mL×3), the combined organic layers were washed with brine (300 mL), dried over anhydrous Na₂SO₄, filtered and concentrated under reduced pressure to give a residue. The residue was purified by column chromatography (SiO₂, DCM/MeOH=100/0 to 4/1) to give compound 653-3 (13 g, 40.62 mmol, 75.66% yield) as a white solid. LCMS: t_{R}=0.410 min, MS (ESI+) m/z=307.09 [M+1]⁺. ¹H NMR (400 MHz, CDCl₃) δ=3.48-3.26 (m, 6H), 2.48-2.38 (m, 2H), 2.37-2.28 (m, 4H), 2.04-1.79 (m, 2H), 1.39 (s, 9H).

Procedure for preparation of compound 653-5. To a solution of compound 653-3 (1.06 g, 3.47 mmol, 1.5 eq) and compound 624-5 (1 g, 2.31 mmol, 1 eq) in DMAc (10 mL) was added DIEA (895.65 mg, 6.93 mmol, 1.21 mL, 3 eq). The mixture was stirred at 100° C. for 12 h. The filtrate was purified by prep-HPLC (6%-36% ACN in water (0.225% FA), 10 min) and lyophilized to give compound 653-5 (348 mg, 504.96 mmol, 21.86% yield) as a white solid. LCMS: t_{R}=0.371 min, MS (ESI+) m/z=659.41 [M+1]⁺

Procedure for preparation of compound 653-6. To a solution of compound 653-5 (248 mg, 376.42 μmol, 1 eq) in DCM (3 mL) was added HCl/dioxane (4 M, 470.53 uL, 5 eq). The mixture was stirred at 25° C. for 1 h. The reaction mixture was filtered and the filtrate was concentrated under the reduced pressure to give compound 653-6 (371 mg, 491.69 mmol) as a yellow solid. LCMS: t_{R}=0.310 min, MS (ESI+) m/z=559.35 [M+1]⁺

Procedure for preparation of MS-6-053. To a solution of compound 653-7 (100 mg, 178.98 μmol, 1 eq) and compound 535-1 (79.09 mg, 187.93 μmol, 1.05 eq) in DMAc (1 mL) was added DIEA (69.40 mg, 536.94 μmol, 93.53 uL, 3 eq). The mixture was stirred at 90° C. for 12 h. The residue was purified by prep-HPLC (10%-40% ACN in water (0.225% FA), 10 min) and lyophilized to give MS-6-053 (16.89 mg, 17.72 μmol, 9.90% yield) as a yellow solid. LCMS: t_{R}=0.349 min, MS (ESI+) m/z=943.5 [M+1]⁺. ¹H NMR (400 MHz, DMSO-d₆) δ=8.75 (s, 1H), 8.62 (d, J=8.4 Hz, 1H), 8.08 (t, J=8.0 Hz, 1H), 7.99 (d, J=7.2 Hz, 1H), 7.62 (t, J=6.0 Hz, 1H), 7.54 (t, J=7.6 Hz, 1H), 7.08 (d, J=8.8 Hz, 1H), 6.99 (d, J=7.2 Hz, 1H), 6.60 (s, 1H), 6.54 (t, J=6.0 Hz, 1H), 5.14 (s, 2H), 5.03 (dd, J=12.6, 5.2 Hz, 1H), 4.64 (q, J=7.2 Hz, 2H), 4.20 (s, 1H), 3.66 (s, 2H), 3.53 (s, 1H), 3.29-3.28 (m, 2H), 3.11 (d, J=6.0 Hz, 2H), 2.78 (s, 3H), 2.67 (s, 1H), 2.65-2.53 (m, 4H), 2.41-2.31 (m, 4H), 2.28-2.14 (m, 13H), 2.06-1.94 (m, 4H), 1.69 (s, 1H), 1.53-1.46 (m, 6H), 1.19 (d, J=6.0 Hz, 3H)

### Example 74

### Synthesis of MS-6-054

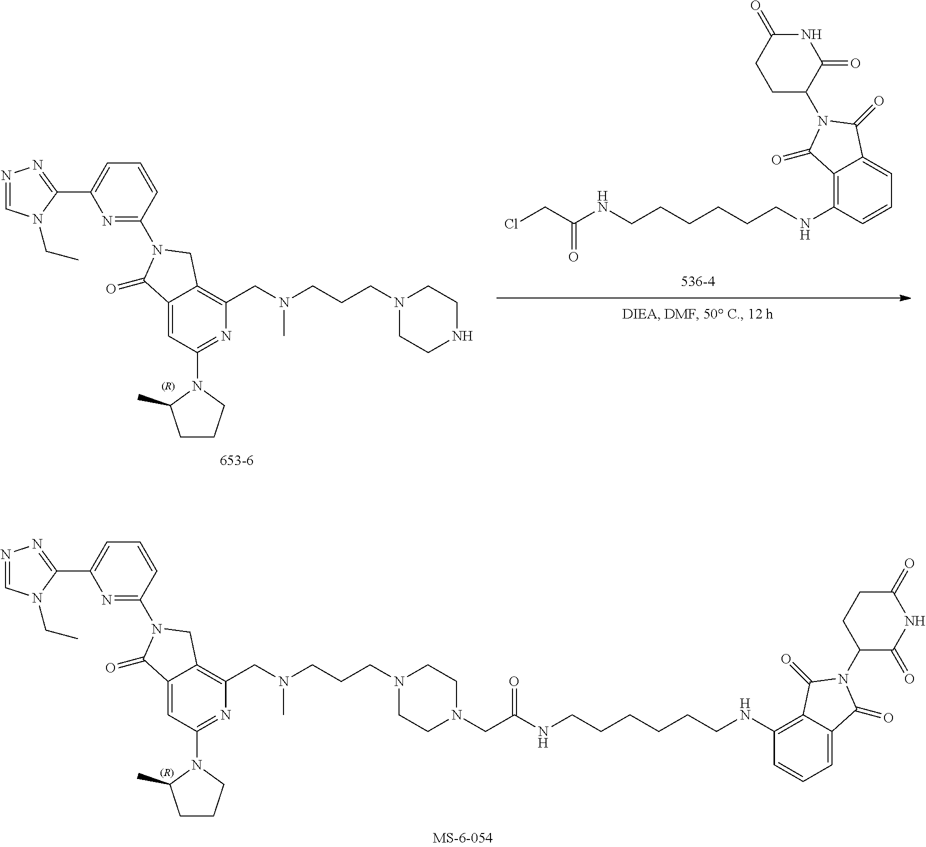

The procedure for preparing compound 536-4 is described in the synthesis of MS-5-036.

The procedure for preparing compound 653-6 is described in the synthesis of MS-6-053.

Procedure for preparation of compound MS-6-054.

To a solution of compound 653-6 (470 mg, 789.68 μmol, 1 eq, HCl) and compound 536-4 (283.59 mg, 631.74 μmol, 0.8 eq) in DMF (4 mL) was added DIEA (306.18 mg, 2.37 mmol, 412.64 uL, 3 eq). The mixture was stirred at 50° C. for 12 h. The reaction was monitored by LCMS, LCMS showed compound 653-6 was consumed and the desired mass was detected. The reaction mixture was concentrated under reduced pressure to remove solvent, which was purified by prep-HPLC (45%-75% ACN in water (10 mM NH₄HCO₃), 10 min). The result solution was lyophilized to give compound MS-6-054 (101.69 mg, 102.19 μmol, 12.94% yield, 97.59% purity) was obtained as yellow solid. LCMS: t_{R}=1.330 min, MS (ESI+) m/z=971.5[M+1]⁺. ¹H NMR (400 MHz, DMSO-d₆): δ=11.08 (s, 1H), 8.75 (s, 1H), 8.60 (d, J=8.4 Hz, 1H), 8.09-8.03 (m, 1H), 8.00-7.95 (m, 1H), 7.59-7.52 (m, 2H), 7.05 (d, J=8.6 Hz, 1H), 6.98 (d, J=7.0 Hz, 1H), 6.57 (s, 1H), 6.49 (t, J=5.6 Hz, 1H), 5.10 (s, 2H), 5.04 (dd, J=5.4, 12.8 Hz, 1H), 4.63 (q, J=7.0 Hz, 2H), 4.18 (t, J=5.8 Hz, 1H), 3.64 (s, 2H), 3.52 (t, J=7.4 Hz, 1H), 3.24 (d, J=6.6 Hz, 2H), 3.06 (q, J=6.8 Hz, 2H), 2.91-2.84 (m, 1H), 2.78 (s, 2H), 2.62-2.53 (m, 2H), 2.36 (t, J=7.0 Hz, 2H), 2.27-2.14 (m, 12H), 2.09-1.92 (m, 5H), 1.68 (s, 1H), 1.59-1.51 (m, 4H), 1.47 (t, J=7.2 Hz, 3H), 1.41-1.36 (m, 2H), 1.35-1.20 (m, 5H), 1.18 (d, J=6.0 Hz, 3H).

### Example 75

### Synthesis of MS-6-055

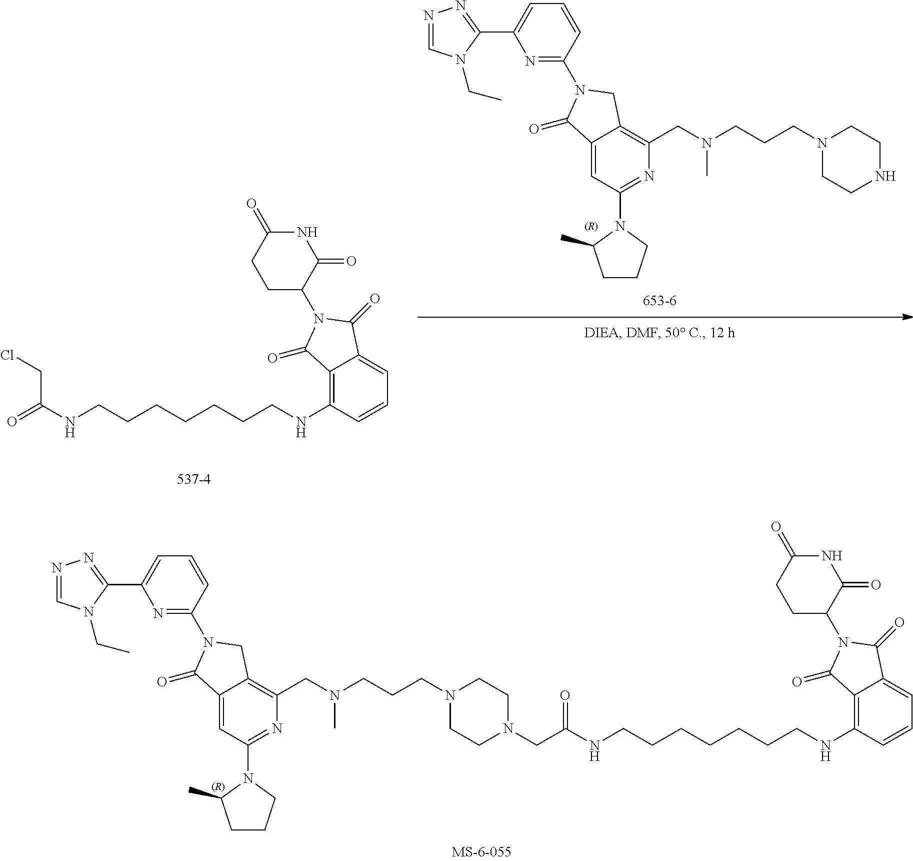

The procedure for preparing compound 537-4 is described in the synthesis of MS-5-037.

Procedure for preparation of compound MS-6-055. To a solution of compound 537-4 (96 mg, 135.53 μmol, 78.88% purity, 1 eq) and compound 653-6 (65.88 mg, 142.31 μmol, 1.05 eq) DMF (1 mL) was added DIEA (52.55 mg, 406.60 μmol, 70.82 uL, 3 eq). The mixture was stirred at 90° C. for 12 h. The residue was purified by prep-HPLC (18%-48% ACN in water (0.225% FA), 9 min) and lyophilized to give MS-6-055 (22.97 mg, 23.32 μmol, 17.20% yield) as a yellow solid. LCMS: t_{R}=0.387 min, MS (ESI+) m/z=985.55 [M+1]⁺. ¹H NMR (400 MHz, DMSO-d₆) δ=11.09 (s, 1H), 8.75 (s, 1H), 8.62 (d, J=8.0 Hz, 1H), 8.10-8.05 (m, 1H), 7.98 (d, J=7.6 Hz, 1H), 7.55 (t, J=7.6 Hz, 2H), 7.05 (d, J=8.4 Hz, 1H), 6.99 (d, J=6.8 Hz, 1H), 6.60-6.57 (m, 1H), 6.53-6.45 (m, 1H), 5.12 (s, 2H), 5.04 (dd, J=12.4, 5.2 Hz, 1H), 4.63 (q, J=7.2 Hz, 2H), 4.19 (s, 1H), 3.73-3.57 (m, 3H), 3.52 (s, 4H), 3.05 (bd, J=6.4 Hz, 2H), 2.94-2.78 (m, 3H), 2.60-2.54 (m, 2H), 2.43-2.28 (m, 5H), 2.26-2.16 (m, 10H), 2.06-1.94 (m, 4H), 1.69 (s, 2H), 1.48 (t, J=7.2 Hz, 3H), 1.42-1.33 (m, 3H), 1.19 (d, J=6.0 Hz, 10H).

### Example 76

### Synthesis of MS-6-058

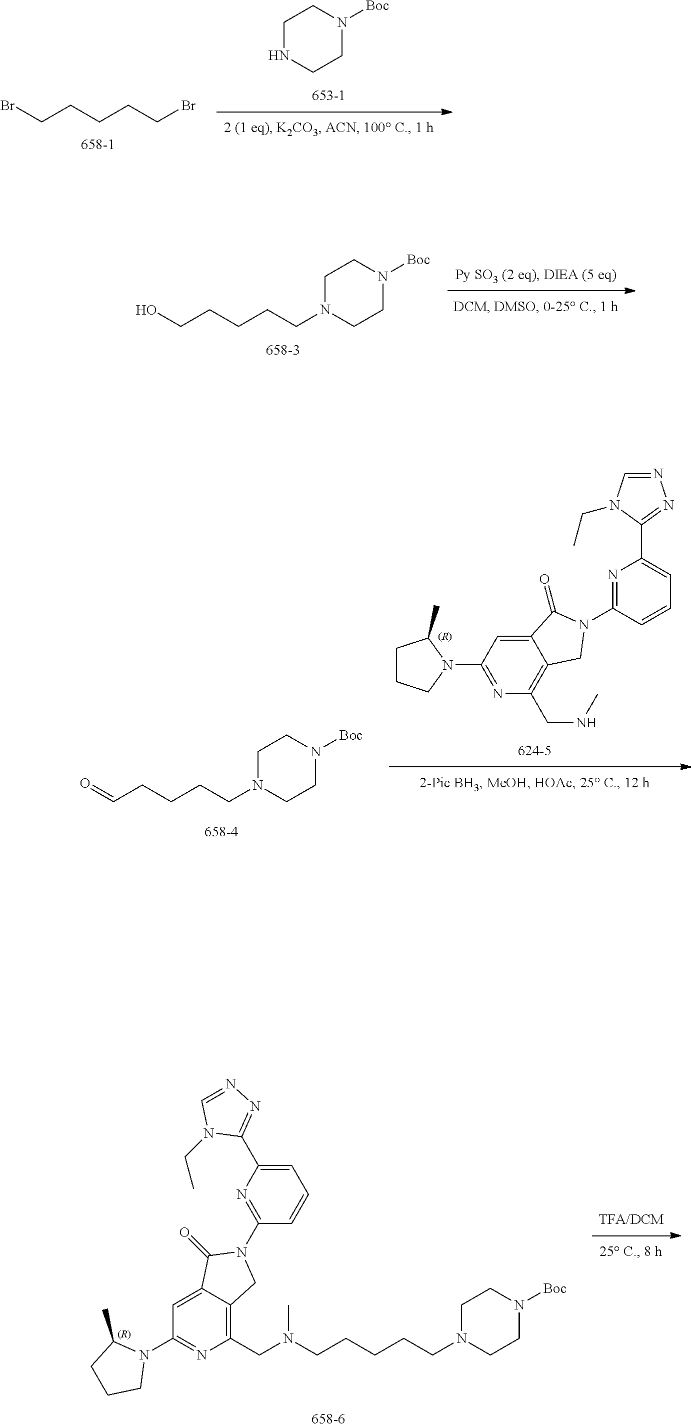

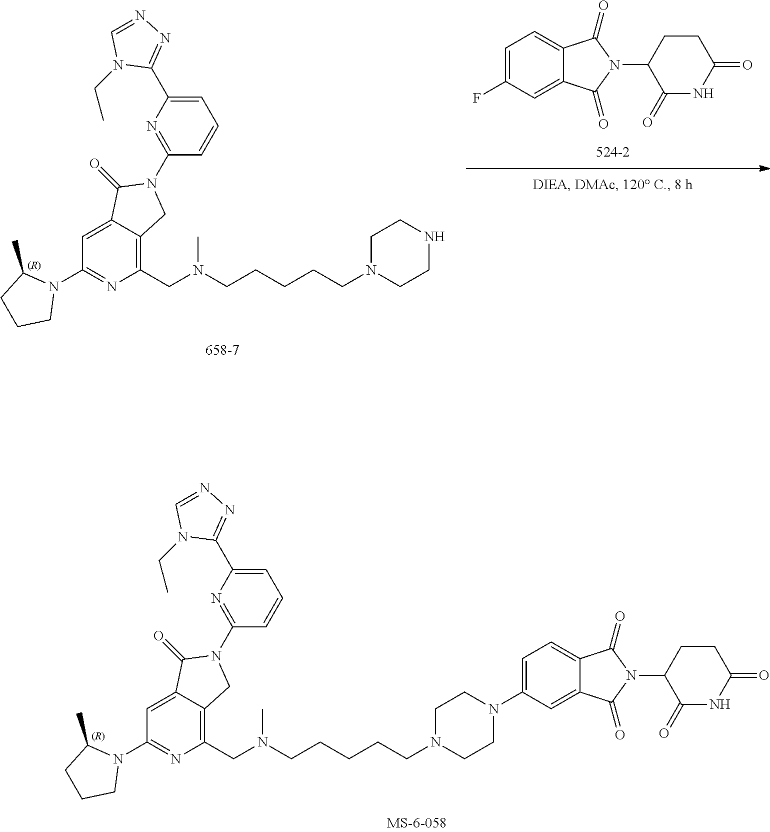

The procedure for preparing compound 624-5 is described in the synthesis of MS-6-027.

Procedure for preparation of compound 658-3. To a solution of compound 658-1 (2 g, 11.97 mmol, 1 eq) and compound 653-1 (2.23 g, 11.97 mmol, 1 eq) in ACN (20 mL) was added K₂CO₃ (4.96 g, 35.92 mmol, 3 eq). The mixture was stirred at 100° C. for 1 h. The reaction mixture was filtered and the filtrate was concentrated under the reduced pressure to give compound 658-3 (3.49 g, crude) as a white solid. LCMS: t_{R}=0.234 min, MS (ESI+) m/z=273.1[M+1]⁺. ¹H NMR (400 MHz, CDCl₃) δ=3.64 (t, J=6.4 Hz, 2H), 3.52-3.34 (m, 6H), 2.85-2.77 (m, 1H), 2.39-2.35 (m, 4H), 1.62-1.50 (m, 4H), 1.46-1.44 (m, 9H), 1.44-1.35 (m, 2H).

Procedure for preparation of compound 658-4. To a solution of compound 658-3 (500 mg, 1.84 mmol, 1 eq) in DCM (5 mL) was added PySO₃ (584.34 mg, 3.67 mmol, 2 eq) in DMSO (0.5 mL) and DIEA (1.19 g, 9.18 mmol, 1.60 mL, 5 eq) at 0° C. The mixture was stirred at 25° C. for 1 h. The reaction mixture was partitioned between DCM (10 mL) and water (10 mL). After quenching the reaction, the reaction mixture was poured into separatory funnel and separated to give compound 658-4 (1.16 g, crude) as a colourless oil.

Procedure for preparation of compound 658-6. To a solution of compound 658-4 (500 mg, 1.16 mmol, 1 eq) and compound 624-5 (625.10 mg, 2.31 mmol, 2 eq) in MeOH (5 mL) and HOAc (0.5 mL) was added 2-Pic BH₃ (247.30 mg, 2.31 mmol, 2 eq). The mixture was stirred at 25° C. for 12 h. The residue was purified by prep-HPLC (58%-88% ACN in water (10 mM NH₄HCO₃), 8 min) and lyophilized to give compound 658-6 (280 mg, 407.64 μmol, 35.26% yield) as a white solid. LCMS: t_{R}=0.373 min, MS (ESI+) m/z=687.6[M+1]⁺

Procedure for preparation of compound 658-7. To a solution of compound 658-6 (250 mg, 363.96 μmol, 1 eq) in TFA (0.2 mL) and DCM (2 mL). The mixture was stirred at 25° C. for 8 h. The mixture was filtered and concentrated in vacuum to give compound 658-7 (260 mg, crude, TFA) as a brown oil. LCMS: t_{R}=0.284 min, MS (ESI+) m/z=587.5[M+1]⁺

Procedure for preparation of MS-6-058. To a solution of compound 658-7 (100 mg, 142.70 μmol, 1 eq, TFA) in DMAc (0.8 mL) was added DIEA (147.54 mg, 1.14 mmol, 198.84 μL, 8 eq) and compound 524-2 (39.42 mg, 142.70 μmol, 1 eq). The mixture was stirred at 120° C. for 8 h. The residue was purified by prep-HPLC (10%-40% ACN in water (0.225% FA), 10 min) and lyophilized to give MS-6-058 (7.17 mg, 7.94 μmol, 5.56% yield, 98.4% purity, FA) as a yellow solid. LCMS: t_{R}=0.321 min, MS (ESI+) m/z=843.6[M+1]⁺. ¹H NMR (400 MHz, DMSO-d₆) δ=11.08 (s, 1H), 8.75 (s, 1H), 8.60 (d, J=8.4 Hz, 1H), 8.25 (s, 1H), 8.10-8.02 (m, 1H), 8.02-7.96 (m, 1H), 7.66 (d, J=8.4 Hz, 1H), 7.26 (s, 1H), 7.18 (d, J=8.4 Hz, 1H), 6.58 (s, 1H), 5.13 (s, 2H), 5.07 (dd, J=5.6, 12.8 Hz, 1H), 4.65 (q, J=7.2 Hz, 2H), 4.19 (d, J=5.6 Hz, 1H), 3.66 (d, J=2.8 Hz, 2H), 3.53 (d, J=6.8 Hz, 2H), 3.29 (s, 4H), 2.91-2.83 (m, 1H), 2.63-2.53 (m, 2H), 2.40-2.29 (m, 6H), 2.23 (s, 3H), 2.16 (t, J=6.8 Hz, 2H), 2.07-1.91 (m, 4H), 1.68 (s, 1H), 1.48 (q, J=6.4 Hz, 5H), 1.38-1.30 (m, 2H), 1.25 (d, J=7.2 Hz, 2H), 1.18 (d, J=6.0 Hz, 3H)

### Example 77

### Synthesis of MS-6-059

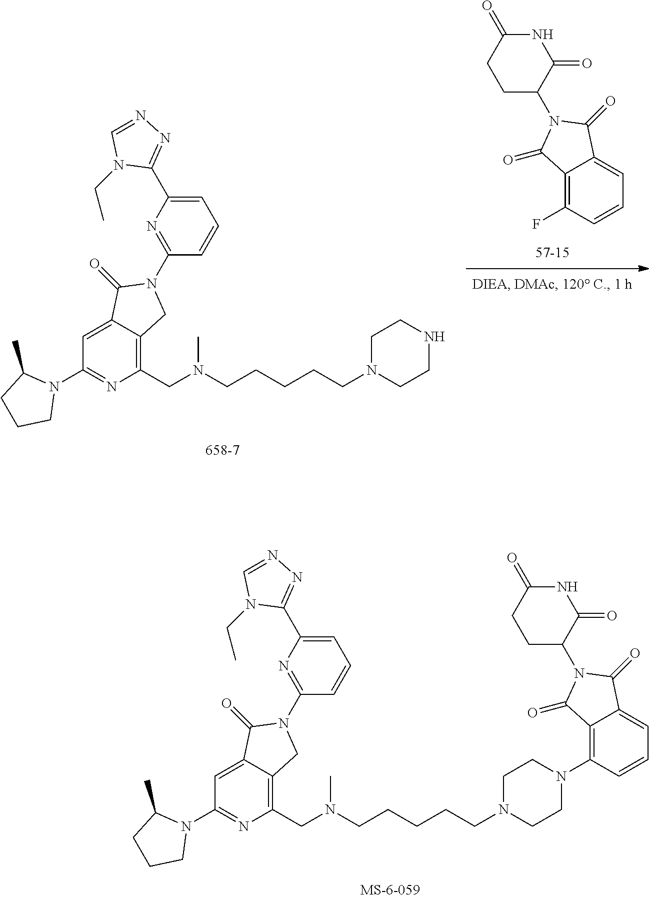 [SMILES: CCn1cnnc1-c1cccc(N2Cc3c(cc(N4CCC[C@H]4C)nc3CN(C)CCCCCN3CCNCC3)C2=O)n1 ; O=C1CCC(N2C(=O)c3cccc(F)c3C2=O)C(=O)N1 ; CCn1cnnc1-c1cccc(N2Cc3c(cc(N4CCC[C@H]4C)nc3CN(C)CCCCCN3CCN(c4cccc5c4C(=O)N(C4CCC(=O)NC4=O)C5=O)CC3)C2=O)n1]

The procedure for preparing compound 658-7 is described in the synthesis of MS-6-058.

Procedure for preparation of MS-6-059. To a solution of compound 658-7 (100 mg, 142.70 μmol, 1 eq, TFA) in DMAc (1 mL) was added DIEA (73.77 mg, 570.78 μmol, 99.42 μL, 4 eq) and compound 57-15 (39.42 mg, 142.70 μmol, 1 eq). The mixture was stirred at 120° C. for 1 h. The residue was purified by prep-HPLC (8%-38% ACN in water (0.225% FA), 10 min) and lyophilized to give MS-6-059 (2.95 mg, 3.32 μmol, 52.93% yield, 100% purity, FA) as a yellow solid. LCMS: t_{R}=0.345 min, MS (ESI+) m/z=843.4[M+1]⁺. ¹H NMR (400 MHz, DMSO-d₆) δ=11.08 (s, 1H), 8.75 (s, 1H), 8.65-8.58 (m, 1H), 8.21 (s, 1H), 8.09-8.02 (m, 1H), 8.01-7.96 (m, 1H), 7.69 (dd, J=8.2, 7.2 Hz, 1H), 7.35 (d, J=7.2 Hz, 1H), 7.24 (d, J=8.6 Hz, 1H), 6.59 (s, 1H), 5.15 (s, 2H), 5.08 (dd, J=12.8, 5.6 Hz, 1H), 4.66 (q, J=7.2 Hz, 2H), 4.25-4.17 (m, 1H), 3.67 (d, J=4.0 Hz, 2H), 3.52 (d, J=2.4 Hz, 2H), 3.17 (s, 4H), 2.88-2.82 (m, 1H), 2.64-2.56 (m, 2H), 2.43-2.35 (m, 6H), 2.24 (s, 3H), 2.19 (t, J=6.8 Hz, 2H), 2.12-1.96 (m, 4H), 1.69 (d, J=4.8 Hz, 1H), 1.54-1.45 (m, 5H), 1.40-1.31 (m, 2H), 1.29-1.22 (m, 2H), 1.19 (d, J=6.4 Hz, 3H).

### Example 78

### Synthesis of MS-6-060

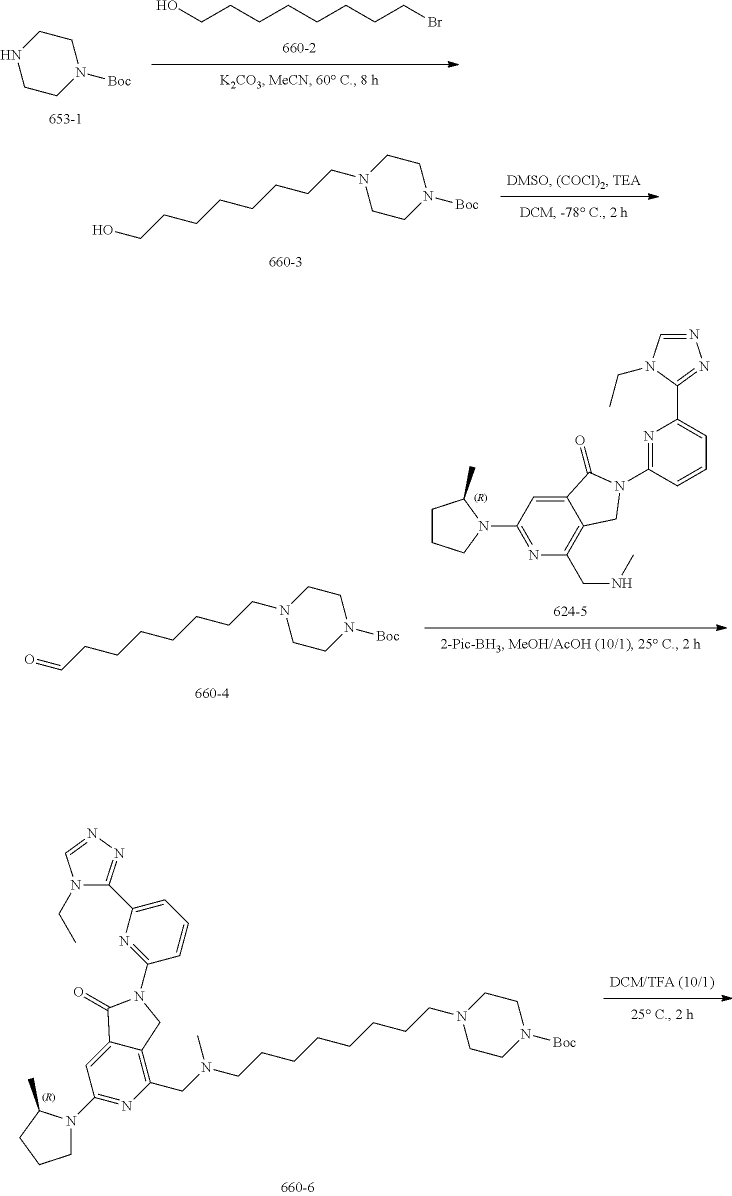

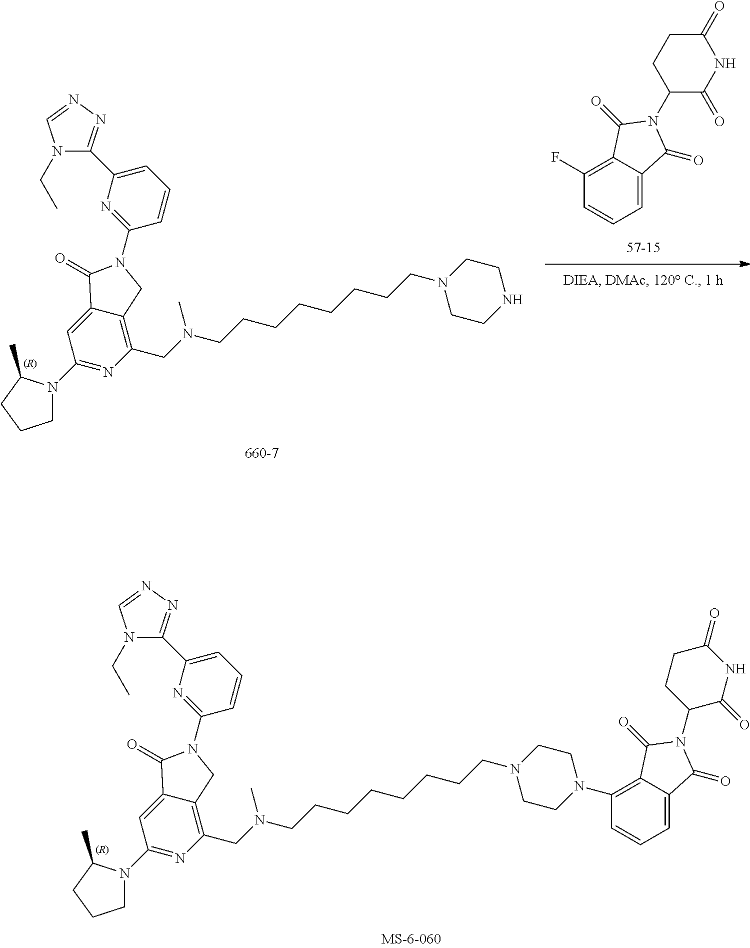

The procedure for preparing compound 624-5 is described in the synthesis of MS-6-024.

Procedure for preparation of compound 660-3. To a solution of compound 653-1 (2 g, 10.74 mmol, 1 eq) in MeCN (20 mL) was added K₂CO₃ (4.45 g, 32.21 mmol, 3 eq) and 8-bromooctan-1-ol (2.25 g, 10.74 mmol, 1.84 mL, 1 eq). The mixture was stirred at 60° C. for 8 h. The mixture was filtered and concentrated in vacuum to give compound 660-3 (3.0 g, crude) as a yellow oil. LCMS: t_{R}=0.278 min, MS (ESI+) m/z=315.3 [M+1]⁺. ¹H NMR (400 MHz, DMSO-d₆) δ=4.20 (t, J=4.8 Hz, 1H), 3.26 (d, J=5.2 Hz, 2H), 3.17-3.24 (m, 4H), 3.06-3.17 (m, 2H), 2.14-2.18 (m, 4H), 1.28 (s, 12H), 1.15 (s, 9H).

Procedure for preparation of compound 660-4. To a solution of DMSO (1.24 g, 15.90 mmol, 1.24 mL, 5 eq) in DCM (20 mL) was added a solution of (COCl)₂ (807.26 mg, 6.36 mmol, 556.73 L, 2 eq) in DCM (20 mL) at −78° C. then stirred for 0.5 h. A solution of compound 660-3 (1 g, 3.18 mmol, 1 eq) in DCM (20 mL) was added dropwise then stirred for 0.5 h. TEA (2.57 g, 25.44 mmol, 3.54 mL, 8 eq) was added and the resulting mixture was stirred at −78° C. for another 1.5 h. The reaction mixture was partitioned between DCM (20 mL) and H₂O (20 mL). The organic phase was separated, washed with brine (10 mL×3), dried over Na₂SO₄, filtered and concentrated under reduced pressure to give compound 660-4 (1 g, crude) as a white oil. ¹H NMR (400 MHz, DMSO-d₆) δ=9.65 (t, J=1.6 Hz, 1H), 3.27 (s, 4H), 2.70 (d, J=4.0 Hz, 2H), 2.40 (m, J=7.2, 1.6 Hz, 2H), 2.24-2.30 (m, 4H), 1.12-1.44 (m, 19H).

Procedure for preparation of compound 660-6. To a solution of compound 660-4 (866.86 mg, 2.77 mmol, 4 eq), AcOH (124.96 mg, 2.08 mmol, 119.01 μL, 3 eq) in MeOH (10 mL) was added compound 624-5 (300 mg, 693.61 μmol, 1 eq) at 25° C. After addition, the mixture was stirred at 25° C. for 1 h. 2-Picoline borane complex (222.57 mg, 2.08 mmol, 3 eq) was added portionwise at 25° C. The resulting mixture was stirred at 25° C. for 1 h. The reaction mixture was diluted with saturated aqueous solution of NaHCO₃ (5 mL) and extracted with EtOAc (10 mL). The combined organic layers were washed with brine (5 mL×3), dried over Na₂SO₄, filtered and concentrated under reduced pressure to give a residue. The residue was purified by prep-HPLC (13%-43% ACN in water (0.225% FA), 10 min). The collected fractions were lyophilized to give compound 660-6 (150 mg, 201.66 μmol, 29.07% yield, 98% purity) as a yellow oil. LCMS: t_{R}=0.373 min, MS (ESI+) m/z=729.6 [M+1]⁺. ¹H NMR (400 MHz, DMSO-d₆) δ=8.74 (s, 1H), 8.63 (dd, J=8.0, 4.0 Hz, 1H), 8.04-8.11 (m, 1H) 7.94-8.04 (m, 1H), 6.59 (s, 1H), 5.14 (s, 2H), 4.64 (m, J=7.2 Hz, 2H), 4.01-4.25 (m, 2H), 3.66 (d, J=5.2 Hz, 2H), 3.53 (dd, J=8.0, 8.0 Hz, 4H), 3.26 (s, 4H), 2.32-2.37 (m, 2H), 2.23 (s, 3H), 2.16-2.21 (m, 4H), 2.10 (d, J=8.0 Hz, 2H), 1.46-1.50 (m, 3H), 1.38 (s, 9H), 1.16-1.27 (m, 10H), 1.10 (s, 6H).

Procedure for preparation of compound 660-7. To a solution of compound 660-6 (150 mg, 205.77 mol, 1 eq) in DCM (15 mL) was added TFA (1.5 mL). The mixture was stirred at 25° C. for 2 h. The mixture was filtered and concentrated in vacuum to give compound 660-7 (300 mg, crude, TFA) as a yellow oil. LCMS: t_{R}=0.313 min, MS (ESI+) m/z=629.5 [M+1]⁺

Procedure for preparation of MS-6-060. To a solution of compound 660-7 (280 mg, 376.91 μmol, 1 eq, TFA) in DMAc (3 mL) was added DIEA (243.57 mg, 1.88 mmol, 328.26 μL, 5 eq) and compound 57-15 (104.11 mg, 376.91 μmol, 1 eq). The mixture was stirred at 120° C. for 1 h. The residue was purified by prep-HPLC (18%-38% ACN in water (0.225% FA), 10 min). The collected fractions were lyophilized to give MS-6-060 (5 mg, 5.54 μmol, 1.47% yield, 98% purity) as a brown solid. LCMS: t_{R}=0.358 min, MS (ESI+) m/z=885.6 [M+1]⁺. ¹H NMR (400 MHz, DMSO-d₆) δ=11.09 (s, 1H), 8.75 (s, 1H), 8.63 (dd, J=8.0, 0.8 Hz, 1H), 8.23 (s, 2H), 8.05-8.11 (m, 1H), 8.00 (dd, J=7.6, 0.8 Hz, 1H), 7.69 (dd, J=8.8, 7.6 Hz, 1H), 7.33 (dd, J=17.2, 7.6 Hz, 2H), 6.59 (s, 1H), 5.14 (s, 2H), 5.09 (dd, J=12.8, 5.6 Hz, 1H), 4.62-4.69 (m, 2H), 4.17-4.22 (m, 1H), 3.66 (d, J=5.6 Hz, 2H), 3.25 (s, 4H), 2.88 (s, 1H), 2.53-2.60 (m, 2H), 2.43-2.47 (m, 4H), 2.32-2.35 (m, 2H), 2.23 (s, 3H), 2.17-2.21 (m, 2H), 1.92-2.10 (m, 5H), 1.69 (dd, J=7.2, 2.8 Hz, 1H), 1.49 (m, J=7.19 Hz, 3H), 1.39-1.46 (m, 2H), 1.25-1.31 (m, 2H), 1.19 (d, J=6.0 Hz, 6H), 1.09-1.15 (m, 6H).

### Example 79

### Synthesis of MS-6-061

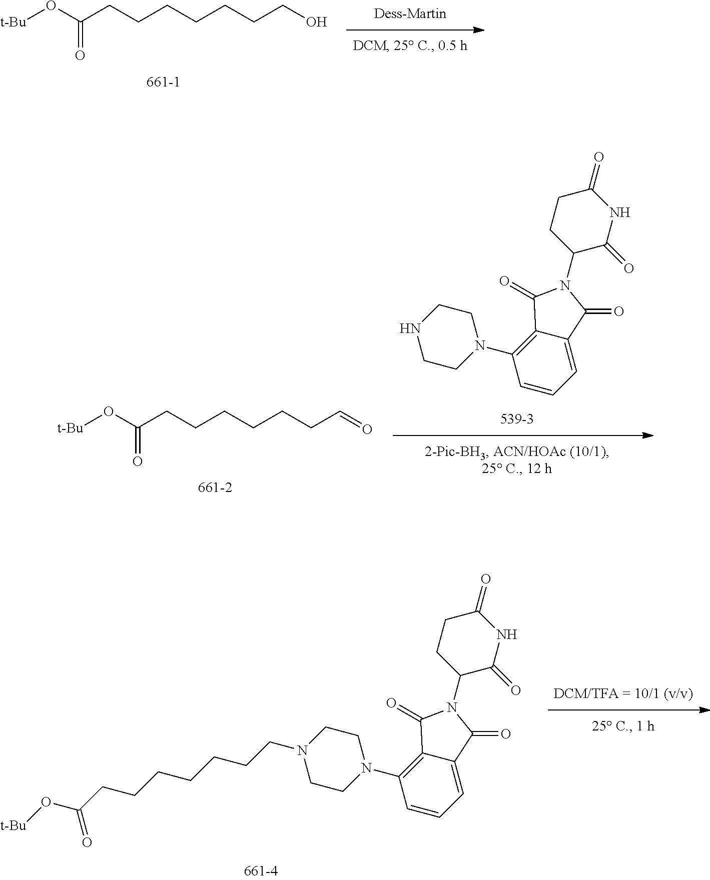 [SMILES: CC(C)(C)OC(=O)CCCCCCCO ; O=C1CCC(N2C(=O)c3cccc(N4CCNCC4)c3C2=O)C(=O)N1 ; CC(C)(C)OC(=O)CCCCCCC=O ; CC(C)(C)OC(=O)CCCCCCCN1CCN(c2cccc3c2C(=O)N(C2CCC(=O)NC2=O)C3=O)CC1]

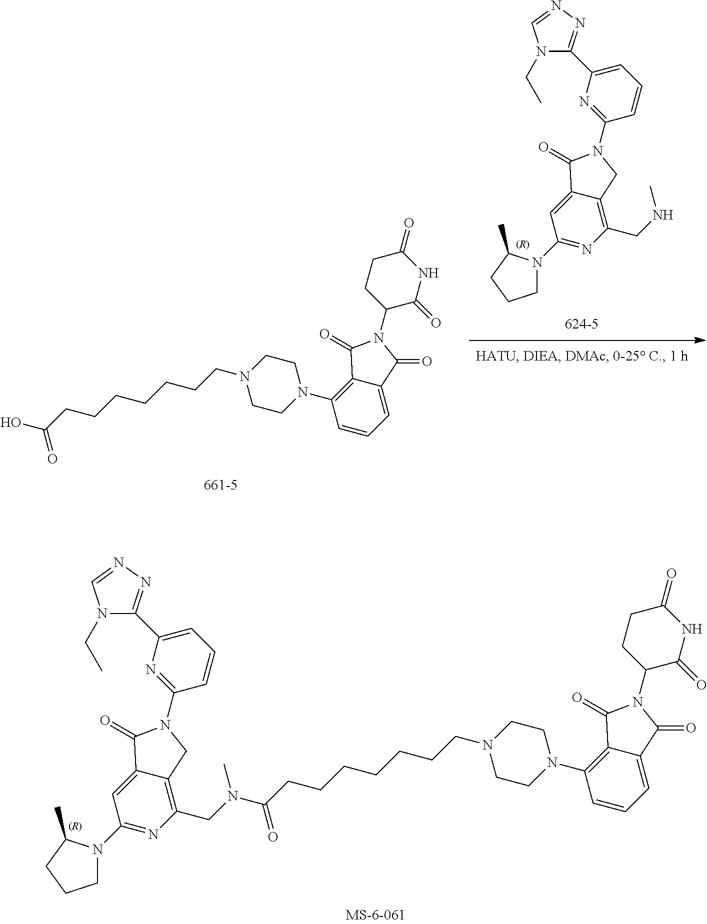

The procedure for preparing compound 539-3 is described in the synthesis of MS-5-039.

Procedure for preparation of compound 661-2. To a solution of compound 661-1 (1 g, 4.62 mmol, 1 eq) in DCM (10 mL) was added Dess-Martin (1.96 g, 4.62 mmol, 1.43 mL, 1 eq). The mixture was stirred at 25° C. for 0.5 h. The reaction mixture was diluted with water (20 mL), and extracted with DCM (10 mL×2). The combined organic layers were dried over anhydrous Na₂SO₄, filtered and concentrated under reduced pressure to give compound 661-2 (1.58 g, crude) as a white solid.

Procedure for preparation of compound 661-4. To a solution of compound 661-2 (208.66 mg, 973.67 μmol, 1 eq) and compound 539-3 (200 mg, 584.20 μmol, 0.6 eq) in ACN (2 mL) and HOAc (0.2 mL) was added 2-Pic-BH₃ (208.29 mg, 1.95 mmol, 2 eq). The residue was purified by prep-HPLC (22%-52% ACN in water (0.225% FA), 10 min) and lyophilized to give compound 661-4 (92 mg, 170.17 μmol, 17.48% yield) as a yellow solid. LCMS: t_{R}=0.370 min, MS (ESI+) m/z=541.2 [M+1]⁺. ¹H NMR (EC6212-387-P1A) (400 MHz, DMSO-d₆): δ=11.08 (s, 1H), 7.70 (dd, J=8.4, 7.2 Hz, 1H), 7.39-7.30 (m, 2H), 5.09 (dd, J=12.8, 5.6 Hz, 1H), 2.95 (d, J=4.0 Hz, 2H), 2.92-2.85 (m, 3H), 2.57 (s, 2H), 2.06-1.97 (m, 1H), 1.52-1.42 (m, 8H), 1.39 (s, 9H), 1.27 (s, 10H).

Procedure for preparation of compound 661-5. To a solution of compound 661-4 (80 mg, 147.97 μmol, 1 eq) in DCM (1 mL) and TFA (0.1 mL). The mixture was stirred at 25° C. for 1 h. The reaction mixture was concentrated under the reduced pressure to give compound 661-5 (80 mg, crude) as a yellow oil. LCMS: t_{R}=0.285 min, MS (ESI+) m/z=485.2 [M+1]⁺

Procedure for preparation of MS-6-061. To a solution of compound 661-5 (80 mg, 165.10 μmol, 1 eq) in DMAc (1 mL) was added HATU (94.17 mg, 247.66 μmol, 1.5 eq) at 0° C. for 0.5 h.

Then DIEA (106.69 mg, 825.52 μmol, 143.79 uL, 5 eq) and compound 624-5 (71.41 mg, 165.10 μmol, 1 eq) was added. The mixture was stirred at 25° C. for 0.5 h. The residue was purified by prep-HPLC (40%-70% ACN in water (10 mM NH₄HCO₃), 8 min) and lyophilized to give MS-6-061 (11.54 mg, 12.40 μmol, 7.51% yield, 96.6% purity) as a yellow solid. LCMS: t_{R}=0.392 min, MS (ESI+) m/z=450.4 [M+1]⁺. ¹H NMR (EC6212-412-P1A) (400 MHz, DMSO-d₆): δ=11.07 (s, 1H), 8.76 (d, J=3.2 Hz, 1H), 8.68-8.56 (m, 1H), 8.15-8.04 (m, 1H), 8.03-7.94 (m, 1H), 7.76-7.62 (m, 1H), 7.39-7.27 (m, 2H), 6.68-6.57 (m, 1H), 5.15-4.99 (m, 3H), 4.71-4.55 (m, 4H), 4.30-4.07 (m, 1H), 3.56-3.49 (m, 1H), 3.26 (s, 5H), 3.11 (s, 2H), 2.93-2.86 (m, 2H), 2.63-2.54 (m, 4H), 2.42-2.31 (m, 3H), 2.29-2.23 (m, 2H), 2.09-1.94 (m, 4H), 1.75-1.64 (m, 1H), 1.53-1.44 (m, 5H), 1.42-1.35 (m, 2H), 1.29-1.13 (m, 10H).

Certain compounds disclosed herein have the structures shown in Table 1.

TABLE 1

| Ex- | Com- | | |
|---|---|---|---|
| am- | pound | | |
| ples | ID | Compound Structure | Compound Name |
| 1 | MS-5- 006 | 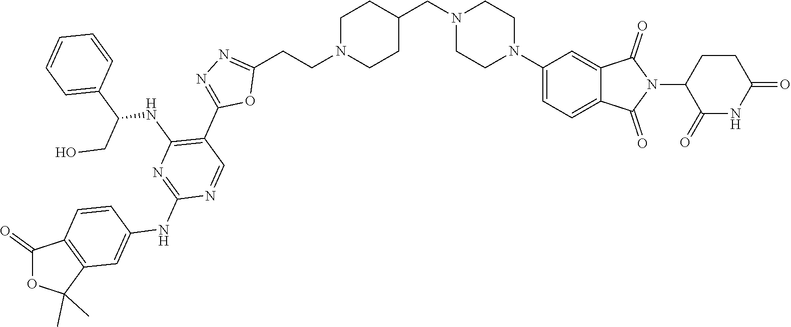 [SMILES: CC1(C)OC(=O)c2ccc(Nc3ncc(-c4nnc(CCN5CCC(CN6CCN(c7ccc8c(c7)C(=O)N(C7CCC(=O)NC7=O)C8=O)CC6)CC5)o4)c(N[C@H](CO)c4ccccc4)n3)cc21] | 5-(4-((1-(2-(5-(2-((3,3-dimethyl-1- oxo-1,3-dihydroisobenzofuran-5- yl)amino)-4-(((S)-2-hydroxy-1- phenylethyl)amino)pyrimidin-5-yl)- 1,3,4-oxadiazol-2-yl)ethyl)piperidin- 4-yl)methyl)piperazin-1-yl)-2-(2,6- dioxopiperidin-3-yl)isoindoline-1,3- dione |
| 2 | MS-5- 007 | 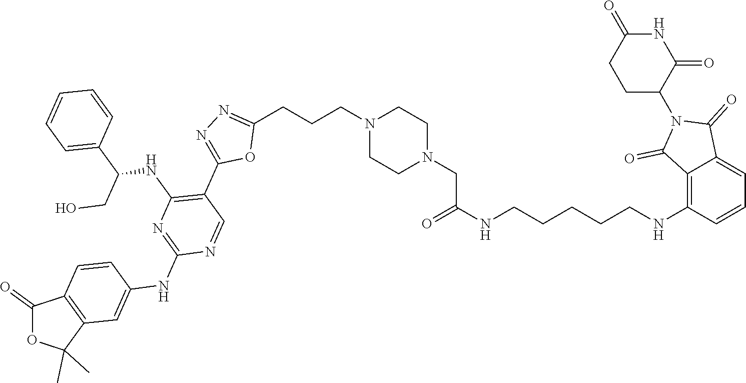 [SMILES: CC1(C)OC(=O)c2ccc(Nc3ncc(-c4nnc(CCCN5CCN(CC(=O)NCCCCCNc6cccc7c6C(=O)N(C6CCC(=O)NC6=O)C7=O)CC5)o4)c(N[C@H](CO)c4ccccc4)n3)cc21] | 2-(4-(3-(5-(2-((3,3-dimethyl-1-oxo- 1,3-dihydroisobenzofuran-5- yl)amino)-4-(((S)-2-hydroxy-1- phenylethyl)amino)pyrimidin-5-yl)- 1,3,4-oxadiazol-2- yl)propyl)piperazin-1-yl)-N-(5-((2- (2,6-dioxopiperidin-3-yl)-1,3- dioxoisoindolin-4- yl)amino)pentyl)acetamide |
| 3 | MS-5- 008 | 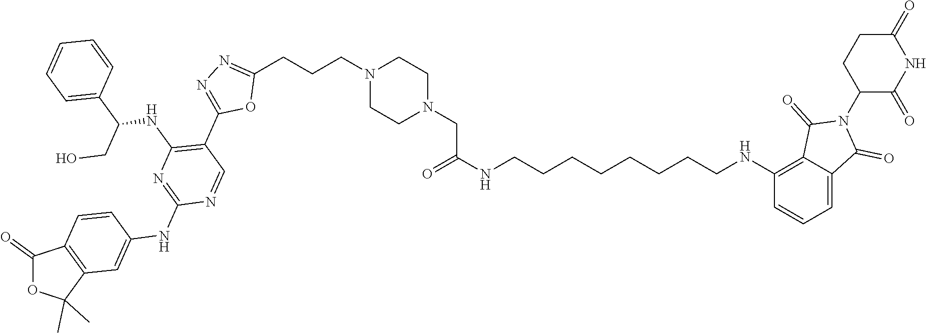 [SMILES: CC1(C)OC(=O)c2ccc(Nc3ncc(-c4nnc(CCCN5CCN(CC(=O)NCCCCCCCCNc6cccc7c6C(=O)N(C6CCC(=O)NC6=O)C7=O)CC5)o4)c(N[C@H](CO)c4ccccc4)n3)cc21] | 2-(4-(3-(5-(2-((3,3-dimethyl-1-oxo- 1,3-dihydroisobenzofuran-5- yl)amino)-4-(((S)-2-hydroxy-1- phenylethyl)amino)pyrimidin-5-yl)- 1,3,4-oxadiazol-2- yl)propyl)piperazin-1-yl)-N-(8-((2- (2,6-dioxopiperidin-3-yl)-1,3- dioxoisoindolin-4- yl)amino)octyl)acetamide |
| 4 | MS-5- 009 | 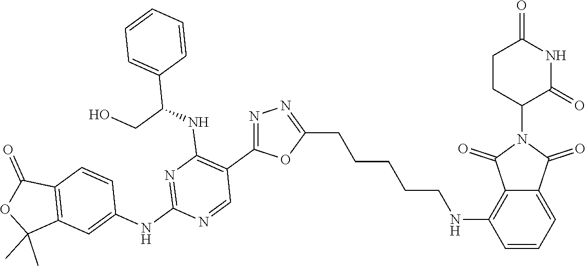 [SMILES: CC1(C)OC(=O)c2ccc(Nc3ncc(-c4nnc(CCCCCNc5cccc6c5C(=O)N(C5CCC(=O)NC5=O)C6=O)o4)c(N[C@H](CO)c4ccccc4)n3)cc21] | 4-((5-(5-(2-((3,3-dimethyl-1-oxo- 1,3-dihydroisobenzofuran-5- yl)amino)-4-(((S)-2-hydroxy-1- phenylethyl)amino)pyrimidin-5-yl)- 1,3,4-oxadiazol-2-yl)pentyl)amino)- 2-(2,6-dioxopiperidin-3- yl)isoindoline-1,3-dione |
| 5 | MS-5- 010 | 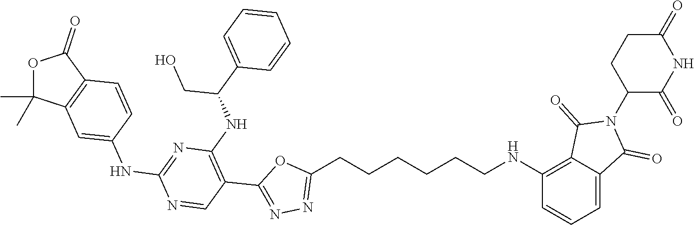 [SMILES: CC1(C)OC(=O)c2ccc(Nc3ncc(-c4nnc(CCCCCCNc5cccc6c5C(=O)N(C5CCC(=O)NC5=O)C6=O)o4)c(N[C@H](CO)c4ccccc4)n3)cc21] | 4-((6-(5-(2-((3,3-dimethyl-1-oxo- 1,3-dihydroisobenzofuran-5- yl)amino)-4-(((S)-2-hydroxy- 1-phenylethyl)amino) pyrimidin-5-yl)-1,3,4- oxadiazol-2-yl)hexyl)amino)- 2-(2,6-dioxopiperidin-3- yl)isoindoline-1,3-dione |
| 6 | MS-5- 011 | 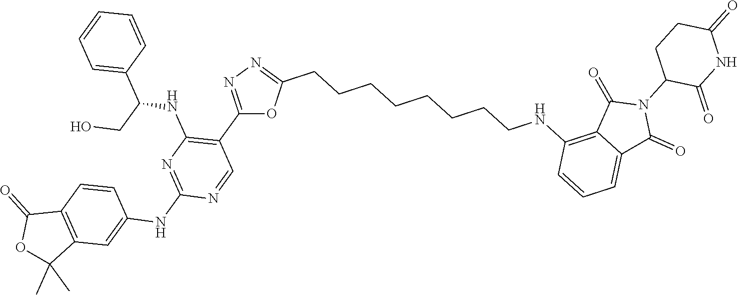 [SMILES: CC1(C)OC(=O)c2ccc(Nc3ncc(-c4nnc(CCCCCCCCNc5cccc6c5C(=O)N(C5CCC(=O)NC5=O)C6=O)o4)c(N[C@H](CO)c4ccccc4)n3)cc21] | 4-((8-(5-(2-((3,3-dimethyl-1-oxo- 1,3-dihydroisobenzofuran-5- yl)amino)-4-(((S)-2-hydroxy-1- phenylethyl)amino)pyrimidin- 5-yl)-1,3,4-oxadiazol-2-yl)octyl) amino)-2-(2,6-dioxopiperidin-3- yl)isoindoline-1,3-dione |
| 7 | MS-5- 015 | 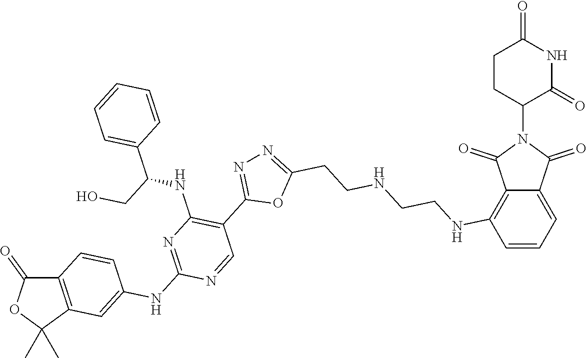 [SMILES: CC1(C)OC(=O)c2ccc(Nc3ncc(-c4nnc(CCNCCNc5cccc6c5C(=O)N(C5CCC(=O)NC5=O)C6=O)o4)c(N[C@H](CO)c4ccccc4)n3)cc21] | 4-((2-((2-(5-(2-((3,3-dimethyl-1- oxo-1,3-dihydroisobenzofuran-5- yl)amino)-4-(((S)-2-hydroxy-1- phenylethyl)amino)pyrimidin-5- yl)-1,3,4-oxadiazol-2-yl)ethyl) amino)ethyl)amino)-2-(2,6- dioxopiperidin-3-yl)isoindoline- 1,3-dione |
| 8 | MS-5- 016 | 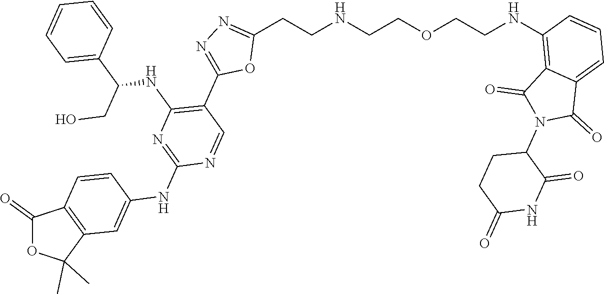 [SMILES: CC1(C)OC(=O)c2ccc(Nc3ncc(-c4nnc(CCNCCOCCNc5cccc6c5C(=O)N(C5CCC(=O)NC5=O)C6=O)o4)c(N[C@H](CO)c4ccccc4)n3)cc21] | 4-((2-(2-((2-(5-(2-((3,3-dimethyl-1- oxo-1,3-dihydroisobenzofuran-5- yl)amino)-4-(((S)-2-hydroxy-1- phenylethyl)amino)pyrimidin-5-yl)- 1,3,4-oxadiazol-2-yl)ethyl) amino)ethoxy)ethyl)amino)- 2-(2,6-dioxopiperidin-3- yl)isoindoline-1,3-dione |
| 9 | MS-5- 017 | 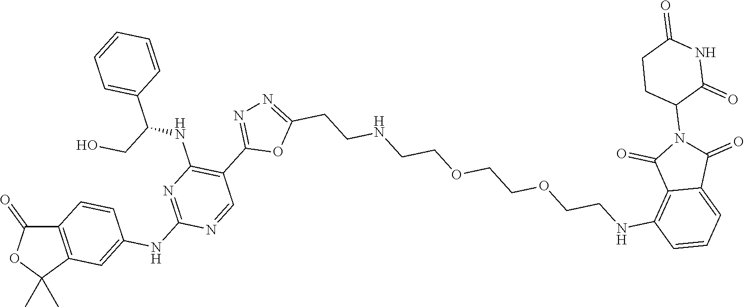 [SMILES: CC1(C)OC(=O)c2ccc(Nc3ncc(-c4nnc(CCNCCOCCOCCNc5cccc6c5C(=O)N(C5CCC(=O)NC5=O)C6=O)o4)c(N[C@H](CO)c4ccccc4)n3)cc21] | 4-((2-(2-(2-((2-(5-(2-((3,3-dimethyl- 1-oxo-1,3-dihydroisobenzofuran-5- yl)amino)-4-(((S)-2-hydroxy-1- phenylethyl)amino)pyrimidin-5-yl)- 1,3,4-oxadiazol-2-yl)ethyl) amino)ethoxy)ethoxy)ethyl) amino)-2-(2,6-dioxopiperidin-3- yl)isoindoline-1,3-dione |
| 10 | MS-5- 018 | 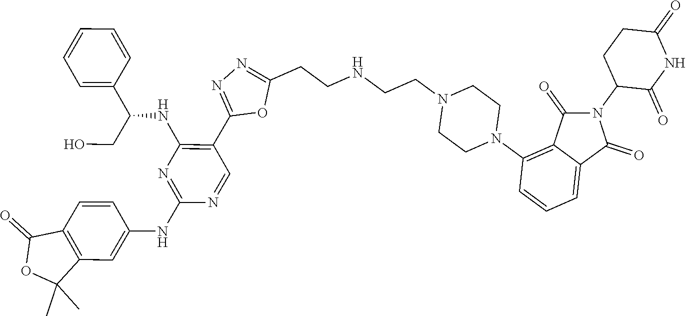 [SMILES: CC1(C)OC(=O)c2ccc(Nc3ncc(-c4nnc(CCNCCN5CCN(c6cccc7c6C(=O)N(C6CCC(=O)NC6=O)C7=O)CC5)o4)c(N[C@H](CO)c4ccccc4)n3)cc21] | 4-(4-(2-((2-(5-(2-((3,3-dimethyl-1- oxo-1,3-dihydroisobenzofuran-5- yl)amino)-4-(((S)-2-hydroxy-1- phenylethyl)amino)pyrimidin-5-yl)- 1,3,4-oxadiazol-2-yl)ethyl) amino)ethyl)piperazin-1-yl)- 2-(2,6-dioxopiperidin-3- yl)isoindoline-1,3-dione |
| 11 | MS-5- 019 | 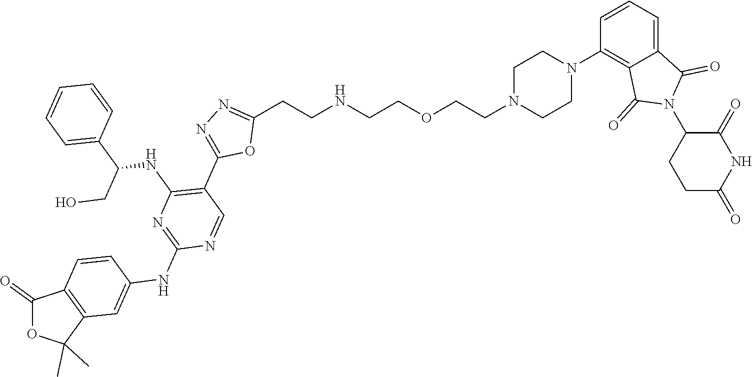 [SMILES: CC1(C)OC(=O)c2ccc(Nc3ncc(-c4nnc(CCNCCOCCN5CCN(c6cccc7c6C(=O)N(C6CCC(=O)NC6=O)C7=O)CC5)o4)c(N[C@H](CO)c4ccccc4)n3)cc21] | 4-(4-(2-(2-((2-(5-(2-((3,3-dimethyl- 1-oxo-1,3-dihydroisobenzofuran-5- yl)amino)-4-(((S)-2-hydroxy-1- phenylethyl)amino)pyrimidin-5-yl)- 1,3,4-oxadiazol-2-yl)ethyl) amino)ethoxy)ethyl)piperazin- 1-yl)-2-(2,6-dioxopiperidin-3- yl)isoindoline-1,3-dione |
| 12 | MS-5- 021 | 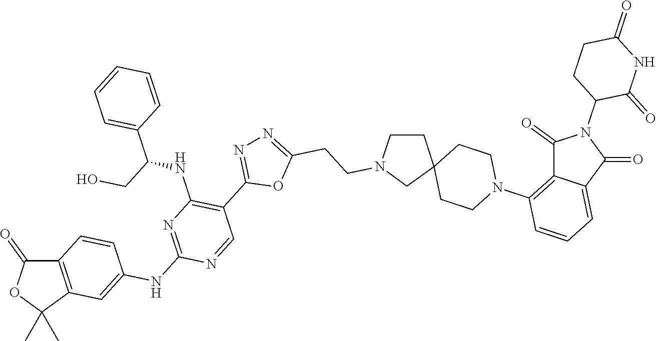 [SMILES: CC1(C)OC(=O)c2ccc(Nc3ncc(-c4nnc(CCN5CCC6(CCN(c7cccc8c7C(=O)N(C7CCC(=O)NC7=O)C8=O)CC6)C5)o4)c(N[C@H](CO)c4ccccc4)n3)cc21] | 4-(2-(2-(5-(2-((3,3-dimethyl-1-oxo- 1,3-dihydroisobenzofuran-5- yl)amino)-4-(((S)-2-hydroxy-1- phenylethyl)amino)pyrimidin-5-yl)- 1,3,4-oxadiazol-2-yl)ethyl)-2,8- diazaspiro[4.5]decan-8-yl)-2-(2,6- dioxopiperidin-3-yl)isoindoline-1,3- dione |
| 13 | MS-5- 022 | 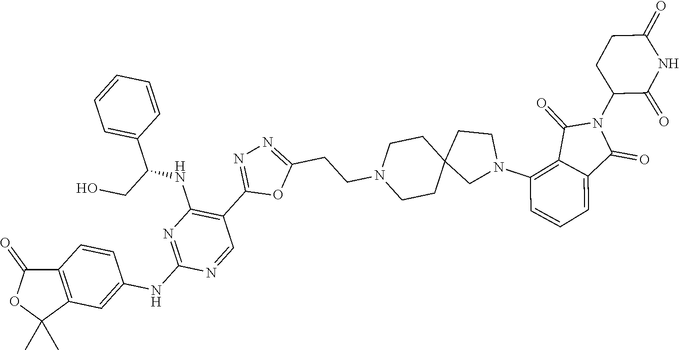 [SMILES: CC1(C)OC(=O)c2ccc(Nc3ncc(-c4nnc(CCN5CCC6(CC5)CCN(c5cccc7c5C(=O)N(C5CCC(=O)NC5=O)C7=O)C6)o4)c(N[C@H](CO)c4ccccc4)n3)cc21] | 4-(8-(2-(5-(2-((3,3-dimethyl-1-oxo- 1,3-dihydroisobenzofuran-5- yl)amino)-4-(((S)-2-hydroxy-1- phenylethyl)amino)pyrimidin-5-yl)- 1,3,4-oxadiazol-2-yl)ethyl)-2,8- diazaspiro[4.5]decan-2-yl)-2-(2,6- dioxopiperidin-3-yl)isoindoline-1,3- dione |
| 14 | MS-5- 024 | 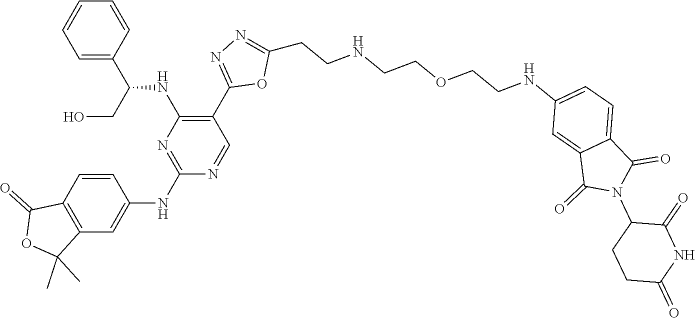 [SMILES: CC1(C)OC(=O)c2ccc(Nc3ncc(-c4nnc(CCNCCOCCNc5ccc6c(c5)C(=O)N(C5CCC(=O)NC5=O)C6=O)o4)c(N[C@H](CO)c4ccccc4)n3)cc21] | 5-((2-(2-((2-(5-(2-((3,3-dimethyl-1- oxo-1,3-dihydroisobenzofuran-5- yl)amino)-4-(((S)-2-hydroxy-1- phenylethyl)amino)pyrimidin-5-yl)- 1,3,4-oxadiazol-2-yl)ethyl) amino)ethoxy)ethyl)amino)- 2-(2,6-dioxopiperidin-3- yl)isoindoline-1,3-dione |
| 15 | MS-5- 026 | 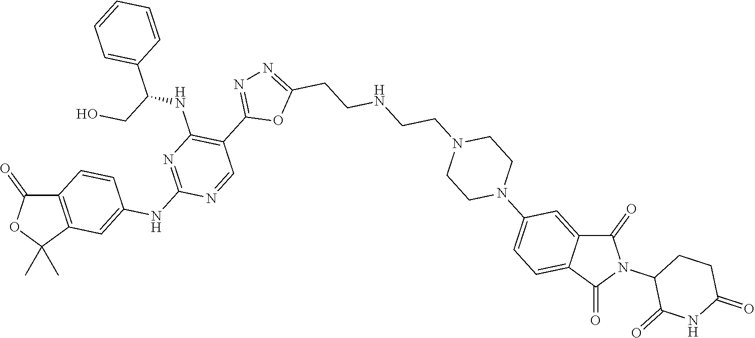 [SMILES: CC1(C)OC(=O)c2ccc(Nc3ncc(-c4nnc(CCNCCN5CCN(c6ccc7c(c6)C(=O)N(C6CCC(=O)NC6=O)C7=O)CC5)o4)c(N[C@H](CO)c4ccccc4)n3)cc21] | 5-(4-(2-((2-(5-(2-((3,3-dimethyl-1- oxo-1,3-dihydroisobenzofuran-5- yl)amino)-4-(((S)-2-hydroxy-1- phenylethyl)amino)pyrimidin-5-yl)- 1,3,4-oxadiazol-2-yl)ethyl) amino)ethyl)piperazin-1-yl)- 2-(2,6-dioxopiperidin-3- yl)isoindoline-1,3-dione |
| 16 | MS-5- 027 | 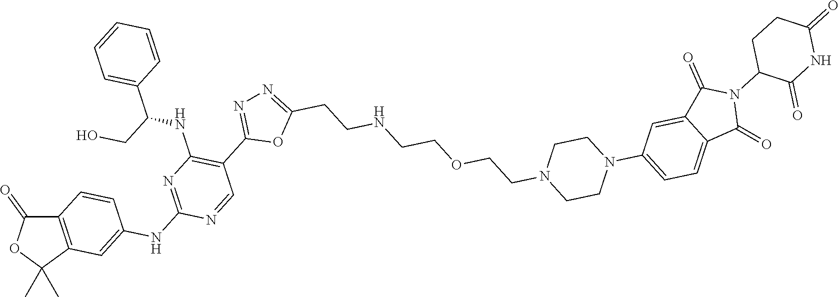 [SMILES: CC1(C)OC(=O)c2ccc(Nc3ncc(-c4nnc(CCNCCOCCN5CCN(c6ccc7c(c6)C(=O)N(C6CCC(=O)NC6=O)C7=O)CC5)o4)c(N[C@H](CO)c4ccccc4)n3)cc21] | 5-(4-(2-(2-((2-(5-(2-((3,3-dimethyl- 1-oxo-1,3-dihydroisobenzofuran-5- yl)amino)-4-(((S)-2-hydroxy-1- phenylethyl)amino)pyrimidin-5-yl)- 1,3,4-oxadiazol-2-yl)ethyl) amino)ethoxy)ethyl)piperazin- 1-yl)-2-(2,6-dioxopiperidin-3- yl)isoindoline-1,3-dione |
| 17 | MS-5- 028 | 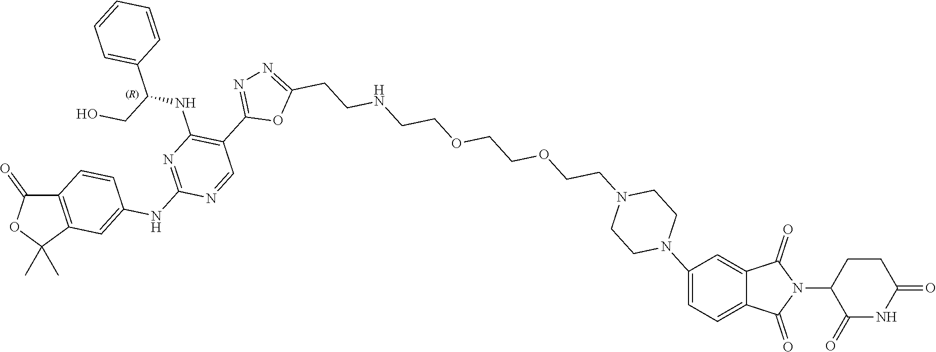 | 4-(4-(2-(2-(2-((2-(5-(2-((3,3- dimethyl-1-oxo-1,3- dihydroisobenzofuran-5- yl)amino)-4-(((S)-2-hydroxy-1- phenylethyl)amino)pyrimidin-5- yl)-1,3,4-oxadiazol-2- yl)ethyl)amino)ethoxy)ethoxy) ethyl)piperazin-1-yl)-2-(2,6- dioxopiperidin-3-yl)isoindoline- 1,3-dione |
| 18 | MS-5- 029 | 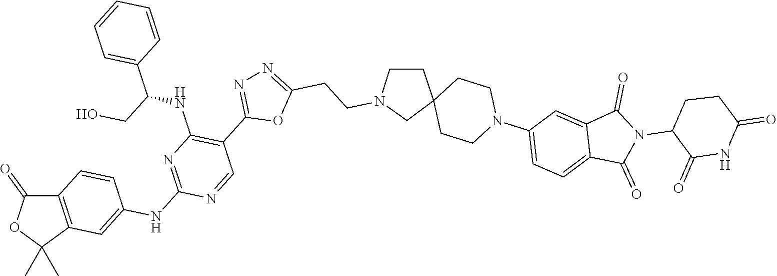 [SMILES: CC1(C)OC(=O)c2ccc(Nc3ncc(-c4nnc(CCN5CCC6(CCN(c7ccc8c(c7)C(=O)N(C7CCC(=O)NC7=O)C8=O)CC6)C5)o4)c(N[C@H](CO)c4ccccc4)n3)cc21] | 5-(2-(2-(5-(2-((3,3-dimethyl-1-oxo- 1,3-dihydroisobenzofuran-5- yl)amino)-4-(((S)-2-hydroxy-1- phenylethyl)amino)pyrimidin-5-yl)- 1,3,4-oxadiazol-2-yl)ethyl)-2,8- diazaspiro[4.5]decan-8-yl)-2-(2,6- dioxopiperidin-3-yl)isoindoline-1,3- dione |
| 19 | MS-5- 030 | 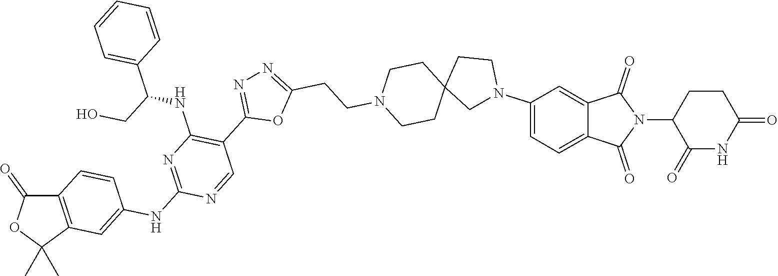 [SMILES: CC1(C)OC(=O)c2ccc(Nc3ncc(-c4nnc(CCN5CCC6(CC5)CCN(c5ccc7c(c5)C(=O)N(C5CCC(=O)NC5=O)C7=O)C6)o4)c(N[C@H](CO)c4ccccc4)n3)cc21] | 5-(8-(2-(5-(2-((3,3-dimethyl-1-oxo- 1,3-dihydroisobenzofuran-5- yl)amino)-4-(((S)-2-hydroxy-1- phenylethyl)amino)pyrimidin-5-yl)- 1,3,4-oxadiazol-2-yl)ethyl)-2,8- diazaspiro[4.5]decan-2-yl)-2-(2,6- dioxopiperidin-3-yl)isoindoline-1,3- dione |
| 20 | MS-5- 033 | 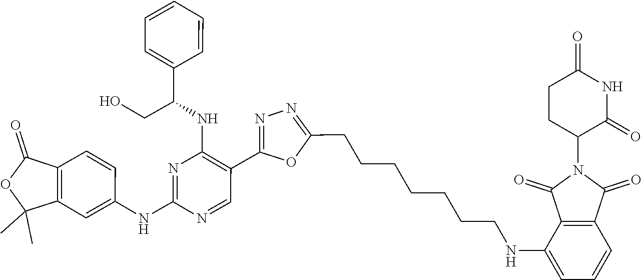 [SMILES: CC1(C)OC(=O)c2ccc(Nc3ncc(-c4nnc(CCCCCCCNc5cccc6c5C(=O)N(C5CCC(=O)NC5=O)C6=O)o4)c(N[C@H](CO)c4ccccc4)n3)cc21] | 4-((7-(5-(2-((3,3-dimethyl-1-oxo- 1,3-dihydroisobenzofuran-5- yl)amino)-4-(((S)-2-hydroxy-1- phenylethyl)amino)pyrimidin-5-yl)- 1,3,4-oxadiazol-2-yl)heptyl)amino)- 2-(2,6-dioxopiperidin-3- yl)isoindoline-1,3-dione |
| 21 | MS-5- 034 | 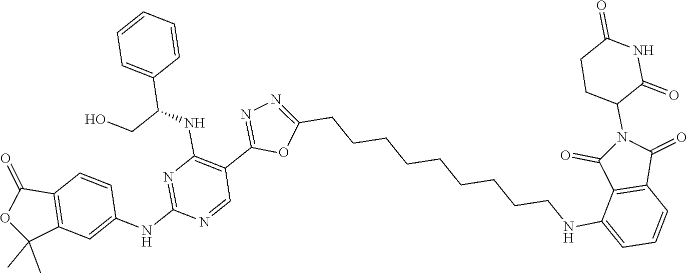 [SMILES: CC1(C)OC(=O)c2ccc(Nc3ncc(-c4nnc(CCCCCCCCCNc5cccc6c5C(=O)N(C5CCC(=O)NC5=O)C6=O)o4)c(N[C@H](CO)c4ccccc4)n3)cc21] | 4-((9-(5-(2-((3,3-dimethyl-1-oxo- 1,3-dihydroisobenzofuran-5- yl)amino)-4-(((S)-2-hydroxy-1- phenylethyl)amino)pyrimidin-5-yl)- 1,3,4-oxadiazol-2-yl)nonyl)amino)- 2-(2,6-dioxopiperidin-3- yl)isoindoline-1,3-dione |
| 22 | MS-5- 035 | 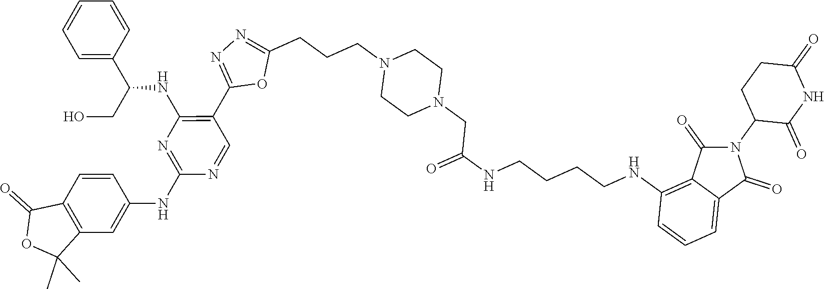 [SMILES: CC1(C)OC(=O)c2ccc(Nc3ncc(-c4nnc(CCCN5CCN(CC(=O)NCCCCNc6cccc7c6C(=O)N(C6CCC(=O)NC6=O)C7=O)CC5)o4)c(N[C@H](CO)c4ccccc4)n3)cc21] | 2-(4-(3-(5-(2-((3,3-dimethyl-1-oxo- 1,3-dihydroisobenzofuran-5- yl)amino)-4-(((S)-2-hydroxy-1- phenylethyl)amino)pyrimidin-5-yl)- 1,3,4-oxadiazol-2- yl)propyl)piperazin-1-yl)-N-(4-((2- (2,6-dioxopiperidin-3-yl)-1,3- dioxoisoindolin-4- yl)amino)butyl)acetamide |
| 23 | MS-5- 036 | 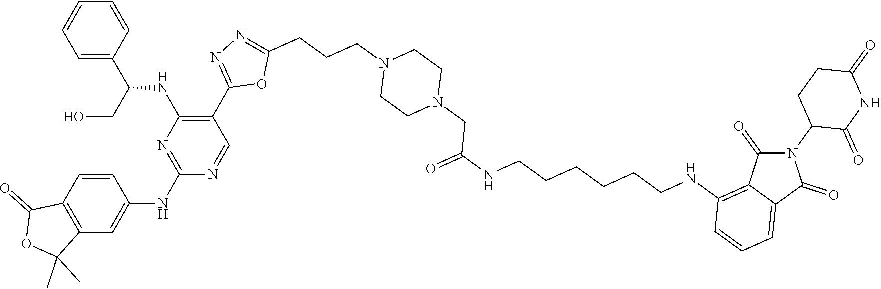 [SMILES: CC1(C)OC(=O)c2ccc(Nc3ncc(-c4nnc(CCCN5CCN(CC(=O)NCCCCCCNc6cccc7c6C(=O)N(C6CCC(=O)NC6=O)C7=O)CC5)o4)c(N[C@H](CO)c4ccccc4)n3)cc21] | 2-(4-(3-(5-(2-((3,3-dimethyl-1-oxo- 1,3-dihydroisobenzofuran-5- yl)amino)-4-(((S)-2-hydroxy-1- phenylethyl)amino)pyrimidin-5-yl)- 1,3,4-oxadiazol-2- yl)propyl)piperazin-1-yl)-N-(6-((2- (2,6-dioxopiperidin-3-yl)-1,3- dioxoisoindolin-4- yl)amino)hexyl)acetamide |
| 24 | MS-5- 037 | 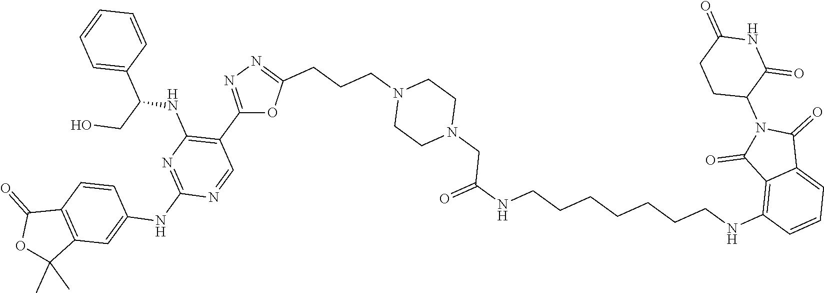 [SMILES: CC1(C)OC(=O)c2ccc(Nc3ncc(-c4nnc(CCCN5CCN(CC(=O)NCCCCCCCNc6cccc7c6C(=O)N(C6CCC(=O)NC6=O)C7=O)CC5)o4)c(N[C@H](CO)c4ccccc4)n3)cc21] | 2-(4-(3-(5-(2-((3,3-dimethyl-1-oxo- 1,3-dihydroisobenzofuran-5- yl)amino)-4-(((S)-2-hydroxy-1- phenylethyl)amino)pyrimidin-5-yl)- 1,3,4-oxadiazol-2-yl)propyl) piperazin-1-yl)-N-(7-((2- (2,6-dioxopiperidin-3-yl)-1,3- dioxoisoindolin-4- yl)amino)heptyl)acetamide |
| 25 | MS-5- 038 | 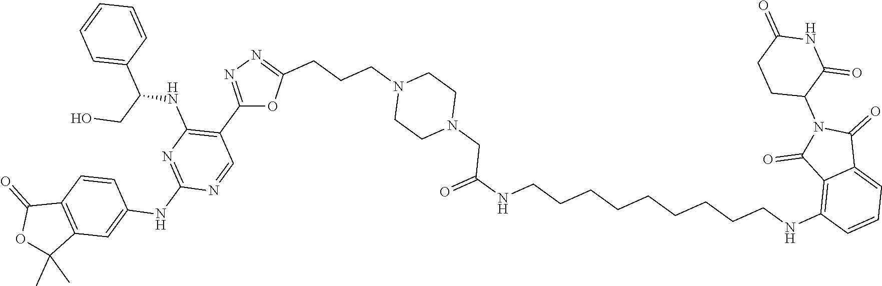 [SMILES: CC1(C)OC(=O)c2ccc(Nc3ncc(-c4nnc(CCCN5CCN(CC(=O)NCCCCCCCCCNc6cccc7c6C(=O)N(C6CCC(=O)NC6=O)C7=O)CC5)o4)c(N[C@H](CO)c4ccccc4)n3)cc21] | 2-(4-(3-(5-(2-((3,3-dimethyl-1-oxo- 1,3-dihydroisobenzofuran-5- yl)amino)-4-(((S)-2-hydroxy-1- phenylethyl)amino)pyrimidin-5-yl)- 1,3,4-oxadiazol-2- yl)propyl)piperazin-1-yl)-N-(9-((2- (2,6-dioxopiperidin-3-yl)-1,3- dioxoisoindolin-4- yl)amino)nonyl)acetamide |
| 26 | MS-5- 039 | 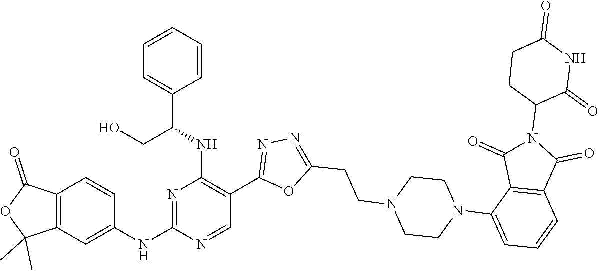 [SMILES: CC1(C)OC(=O)c2ccc(Nc3ncc(-c4nnc(CCN5CCN(c6cccc7c6C(=O)N(C6CCC(=O)NC6=O)C7=O)CC5)o4)c(N[C@H](CO)c4ccccc4)n3)cc21] | 4-(4-(2-(5-(2-((3,3-dimethyl-1-oxo- 1,3-dihydroisobenzofuran-5- yl)amino)-4-(((S)-2-hydroxy-1- phenylethyl)amino)pyrimidin-5-yl)- 1,3,4-oxadiazol-2- yl)ethyl)piperazin-1-yl)-2-(2,6- dioxopiperidin-3-yl)isoindoline-1,3- dione |
| 27 | MS-5- 040 | 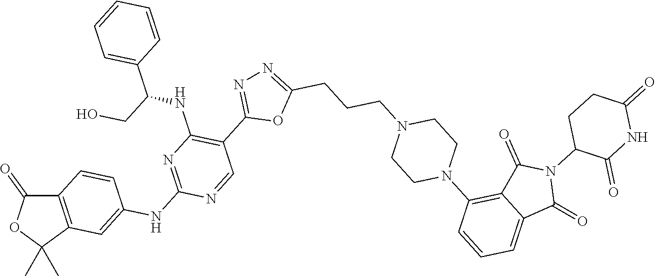 [SMILES: CC1(C)OC(=O)c2ccc(Nc3ncc(-c4nnc(CCCN5CCN(c6cccc7c6C(=O)N(C6CCC(=O)NC6=O)C7=O)CC5)o4)c(N[C@H](CO)c4ccccc4)n3)cc21] | 4-(4-(3-(5-(2-((3,3-dimethyl-1-oxo- 1,3-dihydroisobenzofuran-5- yl)amino)-4-(((S)-2-hydroxy-1- phenylethyl)amino)pyrimidin-5-yl)- 1,3,4-oxadiazol-2- yl)propyl)piperazin-1-yl)-2-(2,6- dioxopiperidin-3-yl)isoindoline-1,3- dione |
| 28 | MS-5- 041 | 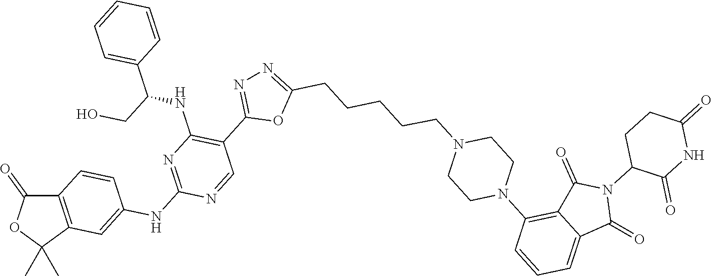 [SMILES: CC1(C)OC(=O)c2ccc(Nc3ncc(-c4nnc(CCCCCN5CCN(c6cccc7c6C(=O)N(C6CCC(=O)NC6=O)C7=O)CC5)o4)c(N[C@H](CO)c4ccccc4)n3)cc21] | 4-(4-(5-(5-(2-((3,3-dimethyl-1-oxo- 1,3-dihydroisobenzofuran-5- yl)amino)-4-(((S)-2-hydroxy-1- phenylethyl)amino)pyrimidin-5-yl)- 1,3,4-oxadiazol-2- yl)pentyl)piperazin-1-yl)-2-(2,6- dioxopiperidin-3-yl)isoindoline-1,3- dione |
| 29 | MS-5- 042 | 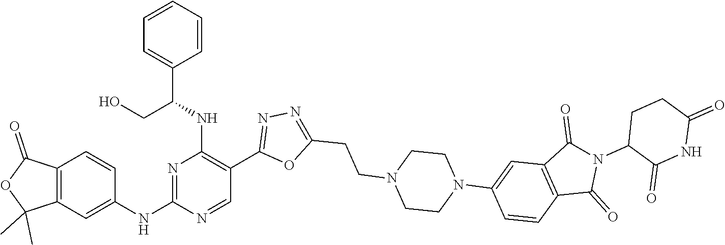 [SMILES: CC1(C)OC(=O)c2ccc(Nc3ncc(-c4nnc(CCN5CCN(c6ccc7c(c6)C(=O)N(C6CCC(=O)NC6=O)C7=O)CC5)o4)c(N[C@H](CO)c4ccccc4)n3)cc21] | 5-(4-(2-(5-(2-((3,3-dimethyl-1-oxo- 1,3-dihydroisobenzofuran-5- yl)amino)-4-(((S)-2-hydroxy-1- phenylethyl)amino)pyrimidin-5-yl)- 1,3,4-oxadiazol-2- yl)ethyl)piperazin-1-yl)-2-(2,6- dioxopiperidin-3-yl)isoindoline-1,3- dione |
| 30 | MS-5- 043 | 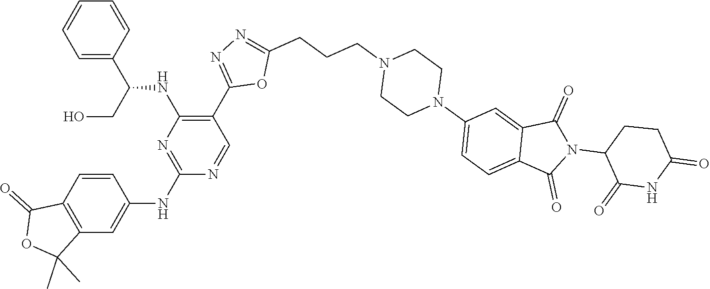 [SMILES: CC1(C)OC(=O)c2ccc(Nc3ncc(-c4nnc(CCCN5CCN(c6ccc7c(c6)C(=O)N(C6CCC(=O)NC6=O)C7=O)CC5)o4)c(N[C@H](CO)c4ccccc4)n3)cc21] | 5-(4-(3-(5-(2-((3,3-dimethyl-1-oxo- 1,3-dihydroisobenzofuran-5- yl)amino)-4-(((S)-2-hydroxy-1- phenylethyl)amino)pyrimidin-5-yl)- 1,3,4-oxadiazol-2- yl)propyl)piperazin-1-yl)-2-(2,6- dioxopiperidin-3-yl)isoindoline-1,3- dione |
| 31 | MS-5- 044 | 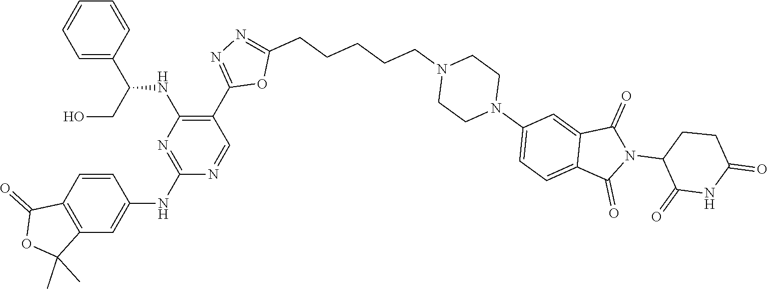 [SMILES: CC1(C)OC(=O)c2ccc(Nc3ncc(-c4nnc(CCCCCN5CCN(c6ccc7c(c6)C(=O)N(C6CCC(=O)NC6=O)C7=O)CC5)o4)c(N[C@H](CO)c4ccccc4)n3)cc21] | 5-(4-(5-(5-(2-((3,3-dimethyl-1-oxo- 1,3-dihydroisobenzofuran-5- yl)amino)-4-(((S)-2-hydroxy-1- phenylethyl)amino)pyrimidin-5-yl)- 1,3,4-oxadiazol-2- yl)pentyl)piperazin-1-yl)-2-(2,6- dioxopiperidin-3-yl)isoindoline-1,3- dione |
| 32 | MS-5- 045 | 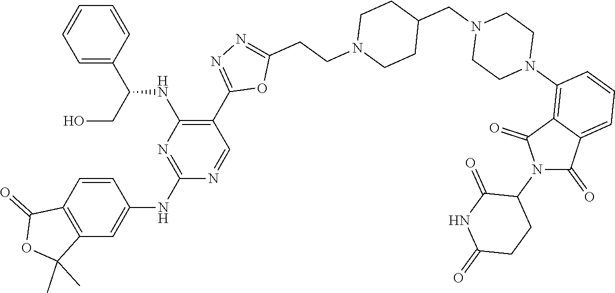 [SMILES: CC1(C)OC(=O)c2ccc(Nc3ncc(-c4nnc(CCN5CCC(CN6CCN(c7cccc8c7C(=O)N(C7CCC(=O)NC7=O)C8=O)CC6)CC5)o4)c(N[C@H](CO)c4ccccc4)n3)cc21] | 4-(4-((1-(2-(5-(2-((3,3-dimethyl-1- oxo-1,3-dihydroisobenzofuran-5- yl)amino)-4-(((S)-2-hydroxy-1- phenylethyl)amino)pyrimidin-5-yl)- 1,3,4-oxadiazol-2-yl)ethyl)piperidin- 4-yl)methyl)piperazin-1-yl)-2-(2,6- dioxopiperidin-3-yl)isoindoline-1,3- dione |
| 33 | MS-5- 046 | 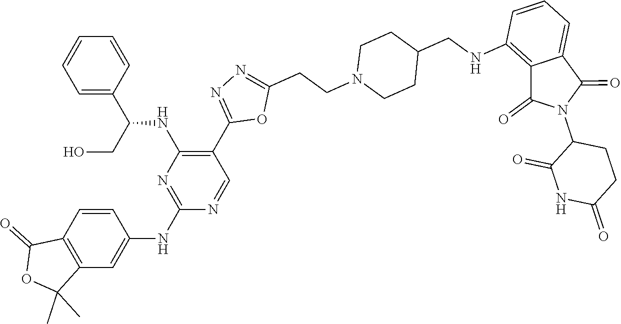 [SMILES: CC1(C)OC(=O)c2ccc(Nc3ncc(-c4nnc(CCN5CCC(CNc6cccc7c6C(=O)N(C6CCC(=O)NC6=O)C7=O)CC5)o4)c(N[C@H](CO)c4ccccc4)n3)cc21] | 4-(((1-(2-(5-(2-((3,3-dimethyl-1- oxo-1,3-dihydroisobenzofuran-5- yl)amino)-4-(((S)-2-hydroxy-1- phenylethyl)amino)pyrimidin-5-yl)- 1,3,4-oxadiazol-2-yl)ethyl)piperidin- 4-yl)methyl)amino)-2-(2,6- dioxopiperidin-3-yl)isoindoline-1,3- dione |
| 34 | MS-6- 003 | 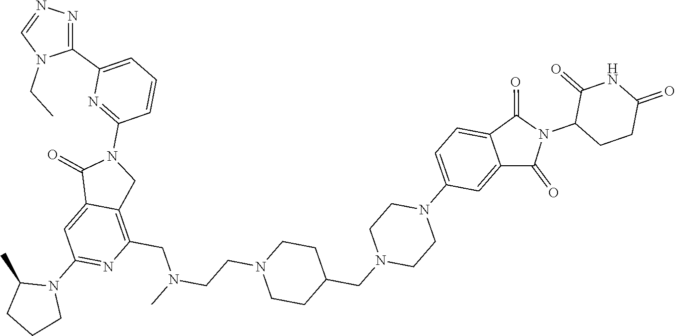 [SMILES: CCn1cnnc1-c1cccc(N2Cc3c(cc(N4CCC[C@H]4C)nc3CN(C)CCN3CCC(CN4CCN(c5ccc6c(c5)C(=O)N(C5CCC(=O)NC5=O)C6=O)CC4)CC3)C2=O)n1] | 2-(2,6-dioxopiperidin-3-yl)-5-(4-((1- (2-(((2-(6-(4-ethyl-4H-1,2,4-triazol- 3-yl)pyridin-2-yl)-6-((R)-2- methylpyrrolidin-1-yl)-1-oxo-2,3- dihydro-1H-pyrrolo[3,4-c]pyridin-4- yl)methyl)(methyl)amino)ethyl) piperidin-4-yl)methyl)piperazin-1- yl)isoindoline-1,3-dione |
| 35 | MS-6- 004 | 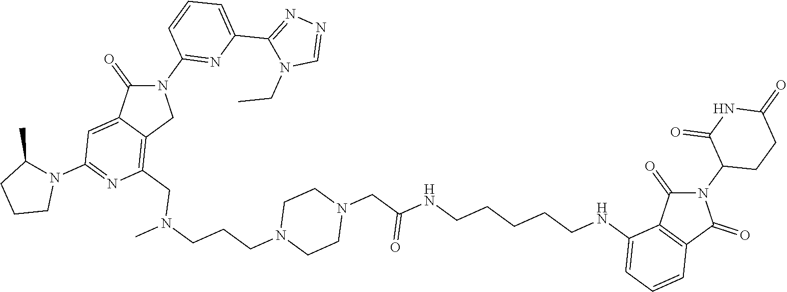 [SMILES: CCn1cnnc1-c1cccc(N2Cc3c(cc(N4CCC[C@H]4C)nc3CN(C)CCCN3CCN(CC(=O)NCCCCCNc4cccc5c4C(=O)N(C4CCC(=O)NC4=O)C5=O)CC3)C2=O)n1] | N-(5-((2-(2,6-dioxopiperidin-3-yl)- 1,3-dioxoisoindolin-4- yl)amino)pentyl)-2-(4-(3- (((2-(6-(4-ethyl-4H-1,2,4-triazol-3- yl)pyridin-2-yl)-6-((R)-2- methylpyrrolidin-1-yl)-1- oxo-2,3-dihydro-1H-pyrrolo[3,4- c]pyridin-4- yl)methyl)(methyl)amino)propyl) piperazin-1-yl)acetamide hydrate |
| 36 | MS-6- 005 | 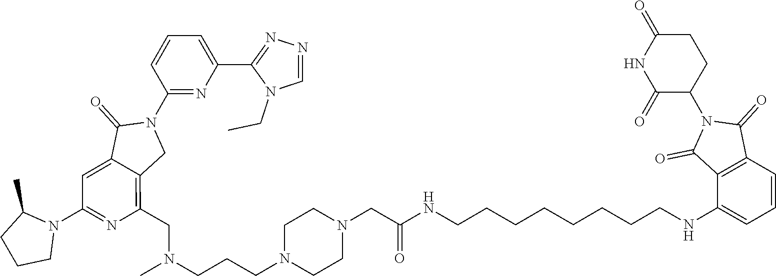 [SMILES: CCn1cnnc1-c1cccc(N2Cc3c(cc(N4CCC[C@H]4C)nc3CN(C)CCCN3CCN(CC(=O)NCCCCCCCCNc4cccc5c4C(=O)N(C4CCC(=O)NC4=O)C5=O)CC3)C2=O)n1] | N-(8-((2-(2,6-dioxopiperidin-3-yl)- 1,3-dioxoisoindolin-4- yl)amino)octyl)-2-(4-(3- (((2-(6-(4-ethyl-4H-1,2,4-triazol- 3-yl)pyridin-2-yl)-6-((R)-2- methylpyrrolidin-1-yl)-1- oxo-2,3-dihydro-1H-pyrrolo[3,4- c]pyridin-4- yl)methyl)(methyl)amino)propyl) piperazin-1-yl)acetamide |
| 37 | MS-6- 006 | 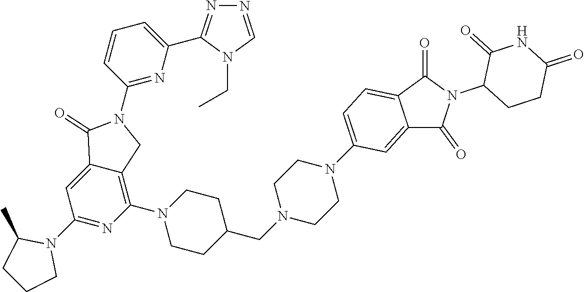 [SMILES: CCn1cnnc1-c1cccc(N2Cc3c(cc(N4CCC[C@H]4C)nc3N3CCC(CN4CCN(c5ccc6c(c5)C(=O)N(C5CCC(=O)NC5=O)C6=O)CC4)CC3)C2=O)n1] | 2-(2,6-dioxopiperidin-3-yl)-5-(4-((1- (2-(6-(4-ethyl-4H-1,2,4-triazol-3- yl)pyridin-2-yl)-6-((R)-2- methylpyrrolidin-1-yl)-1-oxo-2,3- dihydro-1H-pyrrolo[3,4-c]pyridin-4- yl)piperidin-4-yl)methyl)piperazin-1- yl)isoindoline-1,3-dione |
| 38 | MS-6- 007 | 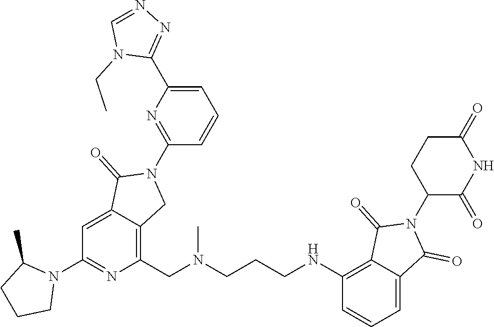 [SMILES: CCn1cnnc1-c1cccc(N2Cc3c(cc(N4CCC[C@H]4C)nc3CN(C)CCCNc3cccc4c3C(=O)N(C3CCC(=O)NC3=O)C4=O)C2=O)n1] | 2-(2,6-dioxopiperidin-3-yl)-4-((3- (((2-(6-(4-ethyl-4H-1,2,4-triazol-3- yl)pyridin-2-yl)-6-((R)-2- methylpyrrolidin-1-yl)-1-oxo-2,3- dihydro-1H-pyrrolo[3,4-c]pyridin-4- yl)methyl)(methyl)amino)propyl) amino)isoindoline-1,3-dione |
| 39 | MS-6- 008 | 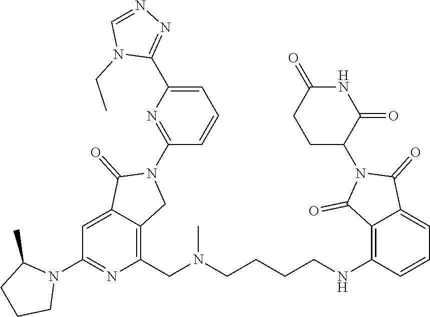 [SMILES: CCn1cnnc1-c1cccc(N2Cc3c(cc(N4CCC[C@H]4C)nc3CN(C)CCCCNc3cccc4c3C(=O)N(C3CCC(=O)NC3=O)C4=O)C2=O)n1] | 2-(2,6-dioxopiperidin-3-yl)-4-((4- (((2-(6-(4-ethyl-4H-1,2,4-triazol-3- yl)pyridin-2-yl)-6-((R)-2- methylpyrrolidin-1-yl)-1-oxo-2,3- dihydro-1H-pyrrolo[3,4-c]pyridin-4- yl)methyl)(methyl)amino)butyl) amino)isoindoline-1,3-dione |
| 40 | MS-6- 009 | 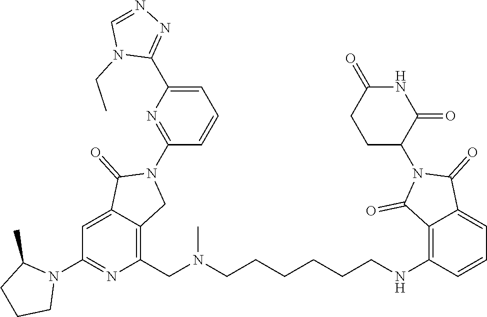 [SMILES: CCn1cnnc1-c1cccc(N2Cc3c(cc(N4CCC[C@H]4C)nc3CN(C)CCCCCCNc3cccc4c3C(=O)N(C3CCC(=O)NC3=O)C4=O)C2=O)n1] | 2-(2,6-dioxopiperidin-3-yl)-4-((6- (((2-(6-(4-ethyl-4H-1,2,4-triazol-3- yl)pyridin-2-yl)-6-((R)-2- methylpyrrolidin-1-yl)-1-oxo-2,3- dihydro-1H-pyrrolo[3,4-c]pyridin-4- yl)methyl)(methyl)amino)hexyl) amino)isoindoline-1,3-dione |
| 41 | MS-6- 013 | 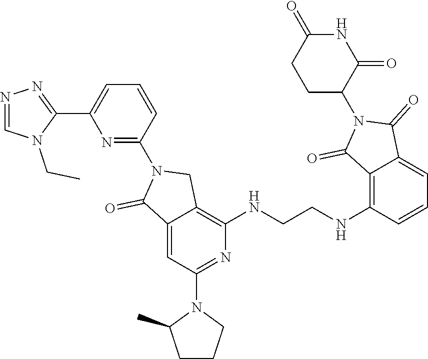 [SMILES: CCn1cnnc1-c1cccc(N2Cc3c(cc(N4CCC[C@H]4C)nc3NCCNc3cccc4c3C(=O)N(C3CCC(=O)NC3=O)C4=O)C2=O)n1] | 2-(2,6-dioxopiperidin-3-yl)-4-((2- ((2-(6-(4-ethyl-4H-1,2,4-triazol-3- yl)pyridin-2-yl)-6-((R)-2- methylpyrrolidin-1-yl)-1-oxo-2,3- dihydro-1H-pyrrolo[3,4-c]pyridin-4- yl)amino)ethyl)amino)isoindoline- 1,3-dione |
| 42 | MS-6- 014 | 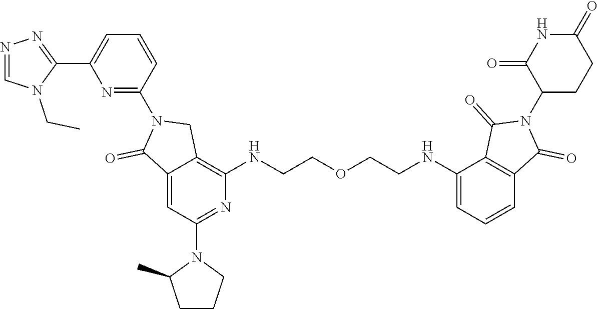 [SMILES: CCn1cnnc1-c1cccc(N2Cc3c(cc(N4CCC[C@H]4C)nc3NCCOCCNc3cccc4c3C(=O)N(C3CCC(=O)NC3=O)C4=O)C2=O)n1] | 2-(2,6-dioxopiperidin-3-yl)-4-((2-(2- ((2-(6-(4-ethyl-4H-1,2,4-triazol-3- yl)pyridin-2-yl)-6-((R)-2- methylpyrrolidin-1-yl)-1-oxo-2,3- dihydro-1H-pyrrolo[3,4-c]pyridin-4- yl)amino)ethoxy)ethyl)amino) isoindoline-1,3-dione |
| 43 | MS-6- 015 | 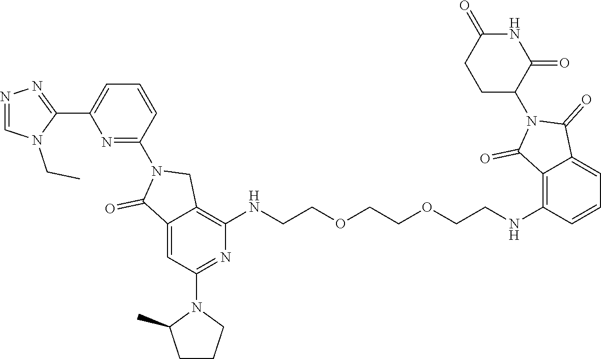 [SMILES: CCn1cnnc1-c1cccc(N2Cc3c(cc(N4CCC[C@H]4C)nc3NCCOCCOCCNc3cccc4c3C(=O)N(C3CCC(=O)NC3=O)C4=O)C2=O)n1] | 2-(2,6-dioxopiperidin-3-yl)-4-((2-(2- (2-((2-(6-(4-ethyl-4H-1,2,4-triazol-3- yl)pyridin-2-yl)-6-((R)-2- methylpyrrolidin-1-yl)-1-oxo-2,3- dihydro-1H-pyrrolo[3,4-c]pyridin-4- yl)amino)ethoxy)ethoxy)ethyl) amino)isoindoline-1,3-dione |
| 44 | MS-6- 016 | 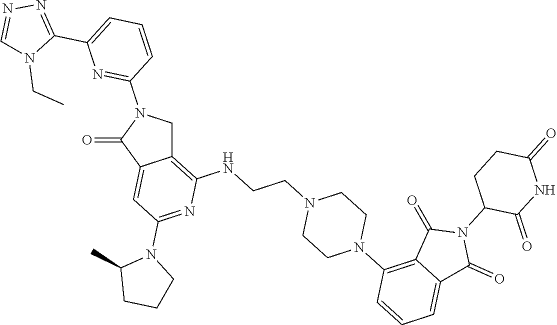 [SMILES: CCn1cnnc1-c1cccc(N2Cc3c(cc(N4CCC[C@H]4C)nc3NCCN3CCN(c4cccc5c4C(=O)N(C4CCC(=O)NC4=O)C5=O)CC3)C2=O)n1] | 2-(2,6-dioxopiperidin-3-yl)-4-(4-(2- ((2-(6-(4-ethyl-4H-1,2,4-triazol-3- yl)pyridin-2-yl)-6-((R)-2- methylpyrrolidin-1-yl)-1-oxo-2,3- dihydro-1H-pyrrolo[3,4-c]pyridin-4- yl)amino)ethyl)piperazin-1- yl)isoindoline-1,3-dione |
| 45 | MS-6- 017 | 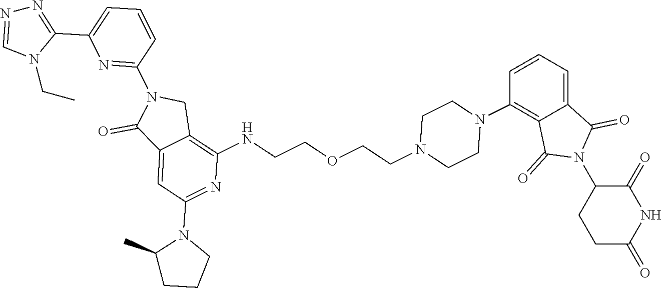 [SMILES: CCn1cnnc1-c1cccc(N2Cc3c(cc(N4CCC[C@H]4C)nc3NCCOCCN3CCN(c4cccc5c4C(=O)N(C4CCC(=O)NC4=O)C5=O)CC3)C2=O)n1] | 2-(2,6-dioxopiperidin-3-yl)-4-(4-(2- (2-((2-(6-(4-ethyl-4H-1,2,4-triazol-3- yl)pyridin-2-yl)-6-((R)-2- methylpyrrolidin-1-yl)-1-oxo-2,3- dihydro-1H-pyrrolo[3,4-c]pyridin-4- yl)amino)ethoxy)ethyl)piperazin-1- yl)isoindoline-1,3-dione |
| 46 | MS-6- 018 | 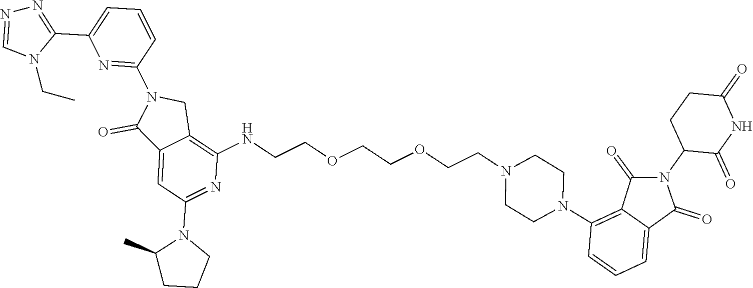 [SMILES: CCn1cnnc1-c1cccc(N2Cc3c(cc(N4CCC[C@H]4C)nc3NCCOCCOCCN3CCN(c4cccc5c4C(=O)N(C4CCC(=O)NC4=O)C5=O)CC3)C2=O)n1] | 2-(2,6-dioxopiperidin-3-yl)-4-(4-(2- (2-(2-((2-(6-(4-ethyl-4H-1,2,4- triazol-3-yl)pyridin-2-yl)-6-((R)-2- methylpyrrolidin-1-yl)-1-oxo-2,3- dihydro-1H-pyrrolo[3,4-c]pyridin-4- yl)amino)ethoxy)ethoxy)ethyl) piperazin-1-yl)isoindoline-1,3-dione |
| 47 | MS-6- 019 | 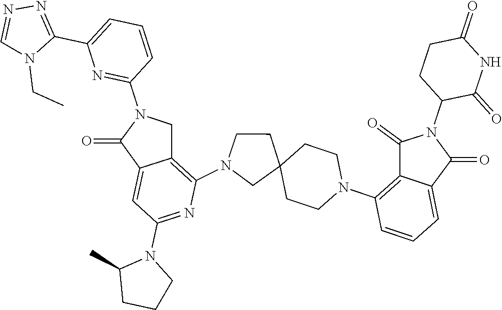 [SMILES: CCn1cnnc1-c1cccc(N2Cc3c(cc(N4CCC[C@H]4C)nc3N3CCC4(CCN(c5cccc6c5C(=O)N(C5CCC(=O)NC5=O)C6=O)CC4)C3)C2=O)n1] | 2-(2,6-dioxopiperidin-3-yl)-4-(2-(2- (6-(4-ethyl-4H-1,2,4-triazol-3- yl)pyridin-2-yl)-6-((R)-2- methylpyrrolidin-1-yl)-1-oxo-2,3- dihydro-1H-pyrrolo[3,4-c]pyridin-4- yl)-2,8-diazaspiro[4.5]decan-8- yl)isoindoline-1,3-dione |
| 48 | MS-6- 020 | 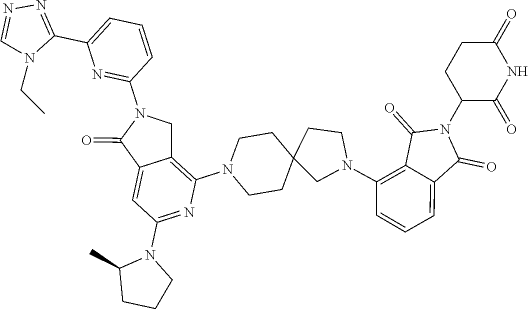 [SMILES: CCn1cnnc1-c1cccc(N2Cc3c(cc(N4CCC[C@H]4C)nc3N3CCC4(CCN(c5cccc6c5C(=O)N(C5CCC(=O)NC5=O)C6=O)C4)CC3)C2=O)n1] | 2-(2,6-dioxopiperidin-3-yl)-4-(8-(2- (6-(4-ethyl-4H-1,2,4-triazol-3- yl)pyridin-2-yl)-6-((R)-2- methylpyrrolidin-1-yl)-1-oxo-2,3- dihydro-1H-pyrrolo[3,4-c]pyridin-4- yl)-2,8-diazaspiro[4.5]decan-2- yl)isoindoline-1,3-dione |
| 49 | MS-6- 021 | 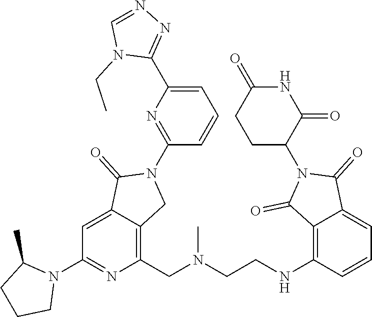 [SMILES: CCn1cnnc1-c1cccc(N2Cc3c(cc(N4CCC[C@H]4C)nc3CN(C)CCNc3cccc4c3C(=O)N(C3CCC(=O)NC3=O)C4=O)C2=O)n1] | 2-(2,6-dioxopiperidin-3-yl)-4-((2- (((2-(6-(4-ethyl-4H-1,2,4-triazol-3- yl)pyridin-2-yl)-6-((R)-2- methylpyrrolidin-1-yl)-1-oxo-2,3- dihydro-1H-pyrrolo[3,4-c]pyridin-4- yl)methyl)(methyl)amino)ethyl) amino)isoindoline-1,3-dione |
| 50 | MS-6- 022 | 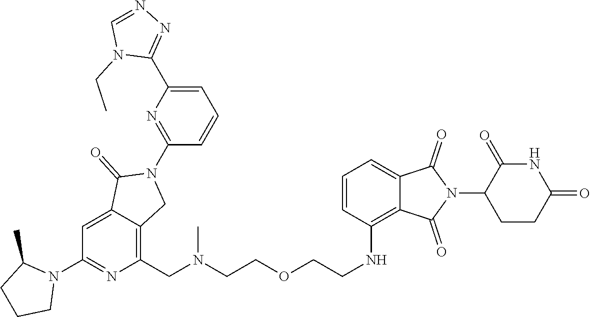 [SMILES: CCn1cnnc1-c1cccc(N2Cc3c(cc(N4CCC[C@H]4C)nc3CN(C)CCOCCNc3cccc4c3C(=O)N(C3CCC(=O)NC3=O)C4=O)C2=O)n1] | 2-(2,6-dioxopiperidin-3-yl)-4-((2-(2- (((2-(6-(4-ethyl-4H-1,2,4-triazol-3- yl)pyridin-2-yl)-6-((R)-2- methylpyrrolidin-1-yl)-1-oxo-2,3- dihydro-1H-pyrrolo[3,4-c]pyridin-4- yl)methyl)(methyl)amino)ethoxy) ethyl)amino)isoindoline-1,3-dione |
| 51 | MS-6- 023 | 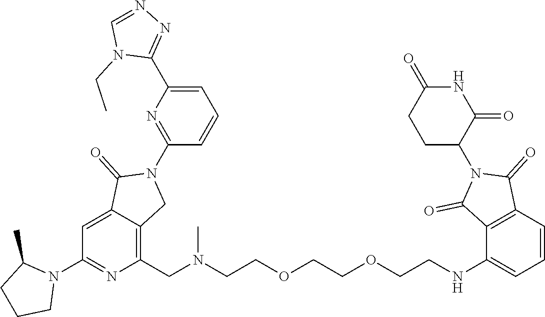 [SMILES: CCn1cnnc1-c1cccc(N2Cc3c(cc(N4CCC[C@H]4C)nc3CN(C)CCOCCOCCNc3cccc4c3C(=O)N(C3CCC(=O)NC3=O)C4=O)C2=O)n1] | 2-(2,6-dioxopiperidin-3-yl)-4-((2-(2- (2-(((2-(6-(4-ethyl-4H-1,2,4-triazol- 3-yl)pyridin-2-yl)-6-((R)-2- methylpyrrolidin-1-yl)-1-oxo-2,3- dihydro-1H-pyrrolo[3,4-c]pyridin-4- yl)methyl)(methyl)amino)ethoxy) ethoxy)ethyl)amino)isoindoline-1,3- dione |
| 52 | MS-6- 024 | 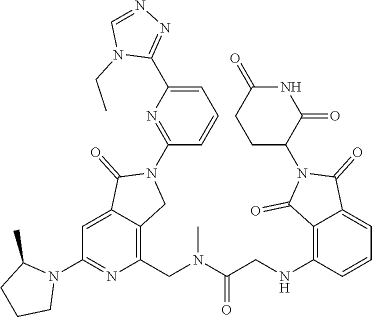 [SMILES: CCn1cnnc1-c1cccc(N2Cc3c(cc(N4CCC[C@H]4C)nc3CN(C)C(=O)CNc3cccc4c3C(=O)N(C3CCC(=O)NC3=O)C4=O)C2=O)n1] | 2-((2-(2,6-dioxopiperidin-3-yl)-1,3- dioxoisoindolin-4-yl)amino)-N-((2- (6-(4-ethyl-4H-1,2,4-triazol-3- yl)pyridin-2-yl)-6-((R)-2- methylpyrrolidin-1-yl)-1-oxo-2,3- dihydro-1H-pyrrolo[3,4-c]pyridin-4- yl)methyl)-N-methylacetamide |
| 53 | MS-6- 025 | 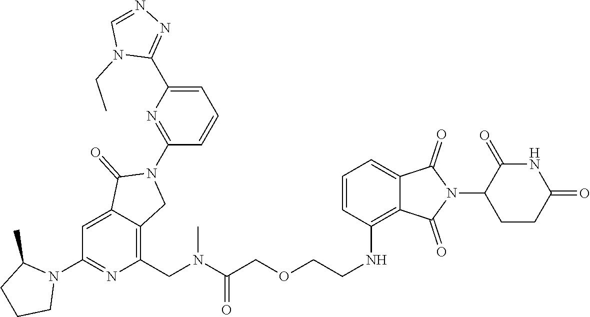 [SMILES: CCn1cnnc1-c1cccc(N2Cc3c(cc(N4CCC[C@H]4C)nc3CN(C)C(=O)COCCNc3cccc4c3C(=O)N(C3CCC(=O)NC3=O)C4=O)C2=O)n1] | 2-(2-((2-(2,6-dioxopiperidin-3-yl)- 1,3-dioxoisoindolin-4- yl)amino)ethoxy)-N-((2- (6-(4-ethyl-4H-1,2,4-triazol-3- yl)pyridin-2-yl)-6-((R)-2- methylpyrrolidin-1-yl)-1-oxo- 2,3-dihydro-1H-pyrrolo[3,4- c]pyridin-4-yl)methyl)-N- methylacetamide |
| 54 | MS-6- 026 | 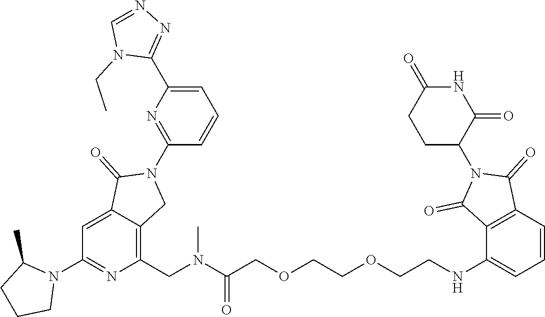 [SMILES: CCn1cnnc1-c1cccc(N2Cc3c(cc(N4CCC[C@H]4C)nc3CN(C)C(=O)COCCOCCNc3cccc4c3C(=O)N(C3CCC(=O)NC3=O)C4=O)C2=O)n1] | 2-(2-(2-((2-(2,6-dioxopiperidin-3- yl)-1,3-dioxoisoindolin-4- yl)amino)ethoxy)ethoxy)-N-((2-(6- (4-ethyl-4H-1,2,4-triazol-3- yl)pyridin-2-yl)-6-((R)-2- methylpyrrolidin-1-yl)-1-oxo-2,3- dihydro-1H-pyrrolo[3,4-c]pyridin-4- yl)methyl)-N-methylacetamide |
| 55 | MS-6- 027 | 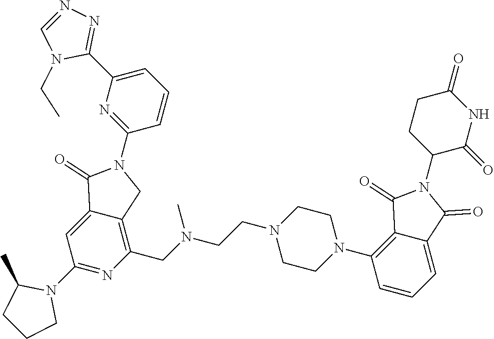 [SMILES: CCn1cnnc1-c1cccc(N2Cc3c(cc(N4CCC[C@H]4C)nc3CN(C)CCN3CCN(c4cccc5c4C(=O)N(C4CCC(=O)NC4=O)C5=O)CC3)C2=O)n1] | 2-(2,6-dioxopiperidin-3-yl)-4-(4-(2- (((2-(6-(4-ethyl-4H-1,2,4-triazol-3- yl)pyridin-2-yl)-6-((R)-2- methylpyrrolidin-1-yl)-1-oxo-2,3- dihydro-1H-pyrrolo[3,4-c]pyridin-4- yl)methyl)(methyl)amino)ethyl) piperazin-1-yl)isoindoline-1,3-dione |
| 56 | MS-6- 028 | 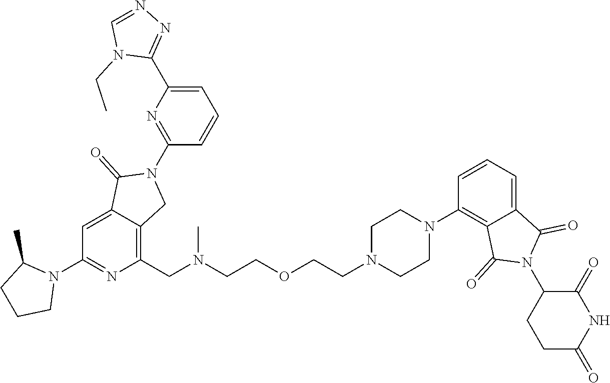 [SMILES: CCn1cnnc1-c1cccc(N2Cc3c(cc(N4CCC[C@H]4C)nc3CN(C)CCOCCN3CCN(c4cccc5c4C(=O)N(C4CCC(=O)NC4=O)C5=O)CC3)C2=O)n1] | 2-(2,6-dioxopiperidin-3-yl)-4-(4-(2- (2-(((2-(6-(4-ethyl-4H-1,2,4-triazol- 3-yl)pyridin-2-yl)-6-((R)-2- methylpyrrolidin-1-yl)-1-oxo-2,3- dihydro-1H-pyrrolo[3,4-c]pyridin-4- yl)methyl)(methyl)amino)ethoxy) ethyl)piperazin-1-yl)isoindoline-1,3- dione |
| 57 | MS-6- 029 | 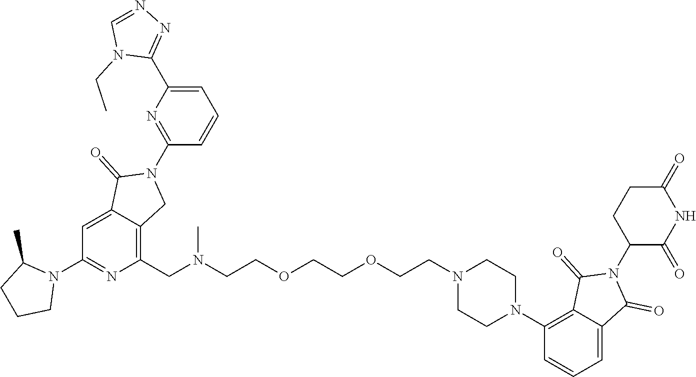 [SMILES: CCn1cnnc1-c1cccc(N2Cc3c(cc(N4CCC[C@H]4C)nc3CN(C)CCOCCOCCN3CCN(c4cccc5c4C(=O)N(C4CCC(=O)NC4=O)C5=O)CC3)C2=O)n1] | 2-(2,6-dioxopiperidin-3-yl)-4-(4-(2- (2-(2-(((2-(6-(4-ethyl-4H-1,2,4- triazol-3-yl)pyridin-2-yl)-6-((R)-2- methylpyrrolidin-1-yl)-1-oxo-2,3- dihydro-1H-pyrrolo[3,4-c]pyridin-4- yl)methyl)(methyl)amino)ethoxy) ethoxy)ethyl)piperazin-1-yl) isoindoline-1,3-dione |
| 58 | MS-6- 037 | 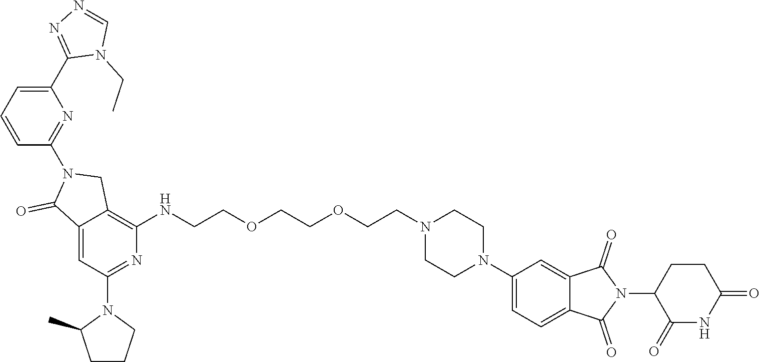 [SMILES: CCn1cnnc1-c1cccc(N2Cc3c(cc(N4CCC[C@H]4C)nc3NCCOCCOCCN3CCN(c4ccc5c(c4)C(=O)N(C4CCC(=O)NC4=O)C5=O)CC3)C2=O)n1] | 2-(2,6-dioxopiperidin-3-yl)-5-(4-(2- (2-(2-((2-(6-(4-ethyl-4H-1,2,4- triazol-3-yl)pyridin-2-yl)-6-((R)-2- methylpyrrolidin-1-yl)-1-oxo-2,3- dihydro-1H-pyrrolo[3,4-c]pyridin-4- yl)amino)ethoxy)ethoxy)ethyl) piperazin-1-yl)isoindoline-1,3-dione |
| 59 | MS-6- 038 | 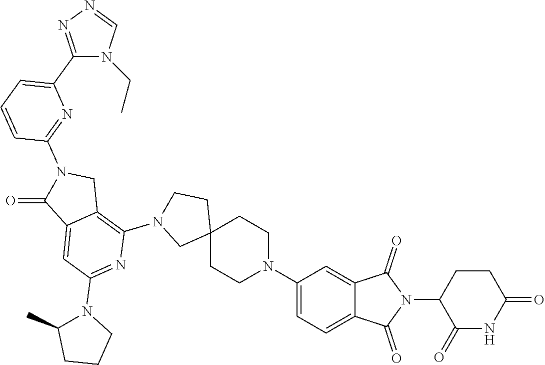 [SMILES: CCn1cnnc1-c1cccc(N2Cc3c(cc(N4CCC[C@H]4C)nc3N3CCC4(CCN(c5ccc6c(c5)C(=O)N(C5CCC(=O)NC5=O)C6=O)CC4)C3)C2=O)n1] | 2-(2,6-dioxopiperidin-3-yl)-5-(2-(2- (6-(4-ethyl-4H-1,2,4-triazol-3- yl)pyridin-2-yl)-6-((R)-2- methylpyrrolidin-1-yl)-1-oxo-2,3- dihydro-1H-pyrrolo[3,4-c]pyridin-4- yl)-2,8-diazaspiro[4.5]decan-8- yl)isoindoline-1,3-dione |
| 60 | MS-6- 039 | 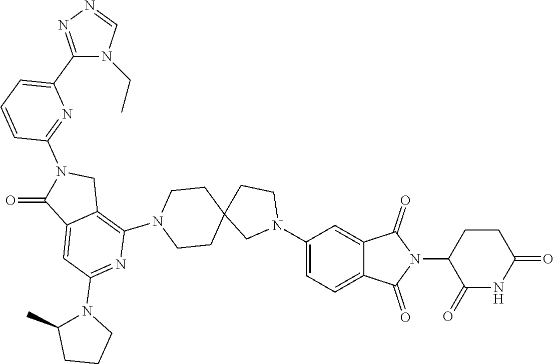 [SMILES: CCn1cnnc1-c1cccc(N2Cc3c(cc(N4CCC[C@H]4C)nc3N3CCC4(CCN(c5ccc6c(c5)C(=O)N(C5CCC(=O)NC5=O)C6=O)C4)CC3)C2=O)n1] | 2-(2,6-dioxopiperidin-3-yl)-5-(8-(2- (6-(4-ethyl-4H-1,2,4-triazol-3- yl)pyridin-2-yl)-6-((R)-2- methylpyrrolidin-1-yl)-1-oxo-2,3- dihydro-1H-pyrrolo[3,4-c]pyridin-4- yl)-2,8-diazaspiro[4.5]decan-2- yl)isoindoline-1,3-dione |
| 61 | MS-6- 040 | 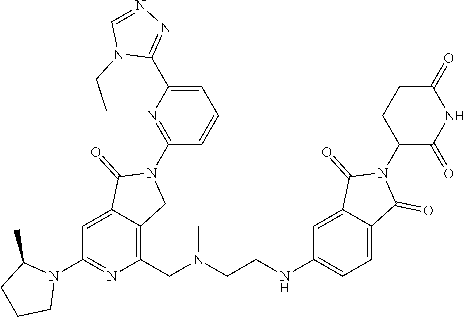 [SMILES: CCn1cnnc1-c1cccc(N2Cc3c(cc(N4CCC[C@H]4C)nc3CN(C)CCNc3ccc4c(c3)C(=O)N(C3CCC(=O)NC3=O)C4=O)C2=O)n1] | 2-(2,6-dioxopiperidin-3-yl)-5-((2- (((2-(6-(4-ethyl-4H-1,2,4-triazol-3- yl)pyridin-2-yl)-6-((R)-2- methylpyrrolidin-1-yl)-1-oxo-2,3- dihydro-1H-pyrrolo[3,4-c]pyridin-4- yl)methyl)(methyl)amino)ethyl) amino)isoindoline-1,3-dione |
| 62 | MS-6- 041 | 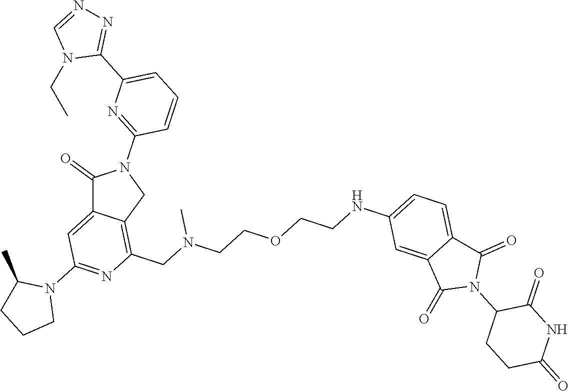 [SMILES: CCn1cnnc1-c1cccc(N2Cc3c(cc(N4CCC[C@H]4C)nc3CN(C)CCOCCNc3ccc4c(c3)C(=O)N(C3CCC(=O)NC3=O)C4=O)C2=O)n1] | 2-(2,6-dioxopiperidin-3-yl)-5-((2-(2- (((2-(6-(4-ethyl-4H-1,2,4-triazol-3- yl)pyridin-2-yl)-6-((R)-2- methylpyrrolidin-1-yl)-1-oxo-2,3- dihydro-1H-pyrrolo[3,4-c]pyridin-4- yl)methyl)(methyl)amino)ethoxy) ethyl)amino)isoindoline-1,3-dione |
| 63 | MS-6- 043 | 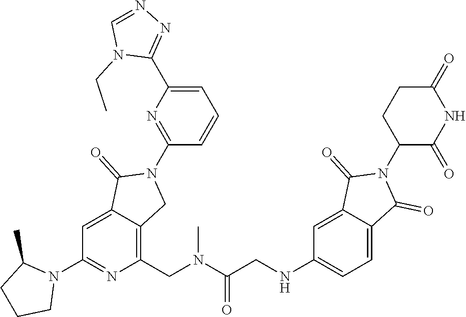 [SMILES: CCn1cnnc1-c1cccc(N2Cc3c(cc(N4CCC[C@H]4C)nc3CN(C)C(=O)CNc3ccc4c(c3)C(=O)N(C3CCC(=O)NC3=O)C4=O)C2=O)n1] | 2-((2-(2,6-dioxopiperidin-3-yl)-1,3- dioxoisoindolin-5-yl)amino)-N-((2- (6-(4-ethyl-4H-1,2,4-triazol-3- yl)pyridin-2-yl)-6-((R)-2- methylpyrrolidin-1-yl)-1-oxo-2,3- dihydro-1H-pyrrolo[3,4-c]pyridin-4- yl)methyl)-N-methylacetamide |
| 64 | MS-6- 044 | 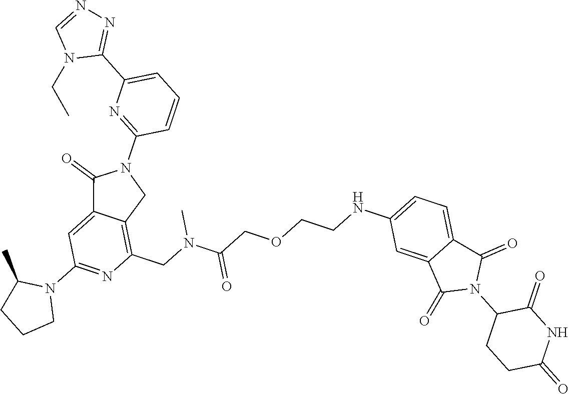 [SMILES: CCn1cnnc1-c1cccc(N2Cc3c(cc(N4CCC[C@H]4C)nc3CN(C)C(=O)COCCNc3ccc4c(c3)C(=O)N(C3CCC(=O)NC3=O)C4=O)C2=O)n1] | 2-(2-((2-(2,6-dioxopiperidin-3-yl)- 1,3-dioxoisoindolin-5- yl)amino)ethoxy)-N-((2- (6-(4-ethyl-4H-1,2,4-triazol-3- yl)pyridin-2-yl)-6-((R)-2- methylpyrrolidin-1-yl)-1-oxo- 2,3-dihydro-1H-pyrrolo[3,4- c]pyridin-4-yl)methyl)-N- methylacetamide |
| 65 | MS-6- 045 | 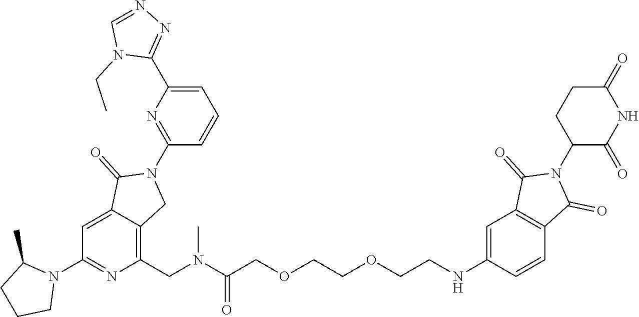 [SMILES: CCn1cnnc1-c1cccc(N2Cc3c(cc(N4CCC[C@H]4C)nc3CN(C)C(=O)COCCOCCNc3ccc4c(c3)C(=O)N(C3CCC(=O)NC3=O)C4=O)C2=O)n1] | 2-(2-(2-((2-(2,6-dioxopiperidin-3- yl)-1,3-dioxoisoindolin-5- yl)amino)ethoxy)ethoxy)-N-((2-(6- (4-ethyl-4H-1,2,4-triazol-3- yl)pyridin-2-yl)-6-((R)-2- methylpyrrolidin-1-yl)-1-oxo-2,3- dihydro-1H-pyrrolo[3,4-c]pyridin-4- yl)methyl)-N-methylacetamide |
| 66 | MS-6- 046 | 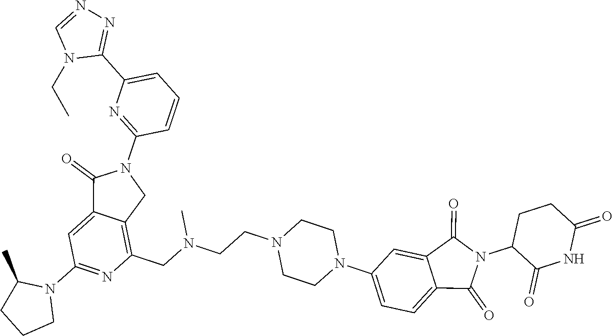 [SMILES: CCn1cnnc1-c1cccc(N2Cc3c(cc(N4CCC[C@H]4C)nc3CN(C)CCN3CCN(c4ccc5c(c4)C(=O)N(C4CCC(=O)NC4=O)C5=O)CC3)C2=O)n1] | 2-(2,6-dioxopiperidin-3-yl)-5-(4-(2- (((2-(6-(4-ethyl-4H-1,2,4-triazol-3- yl)pyridin-2-yl)-6-((R)-2- methylpyrrolidin-1-yl)-1-oxo-2,3- dihydro-1H-pyrrolo[3,4-c]pyridin-4- yl)methyl)(methyl)amino)ethyl) piperazin-1-yl)isoindoline-1,3-dione |
| 67 | MS-6- 047 | 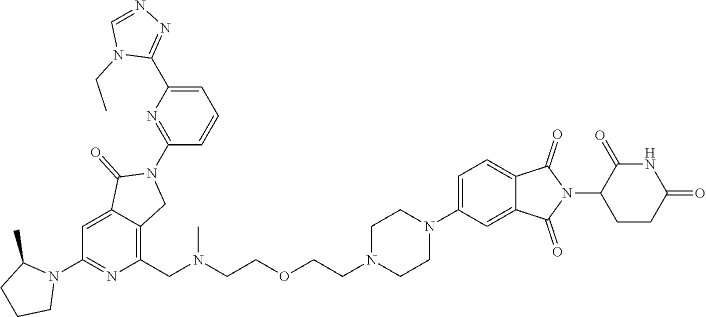 [SMILES: CCn1cnnc1-c1cccc(N2Cc3c(cc(N4CCC[C@H]4C)nc3CN(C)CCOCCN3CCN(c4ccc5c(c4)C(=O)N(C4CCC(=O)NC4=O)C5=O)CC3)C2=O)n1] | 2-(2,6-dioxopiperidin-3-yl)-5-(4-(2- (2-(((2-(6-(4-ethyl-4H-1,2,4-triazol- 3-yl)pyridin-2-yl)-6-((R)-2- methylpyrrolidin-1-yl)-1-oxo-2,3- dihydro-1H-pyrrolo[3,4-c]pyridin-4- yl)methyl)(methyl)amino)ethoxy) ethyl)piperazin-1-yl)isoindoline-1,3- dione |
| 68 | MS-6- 048 | 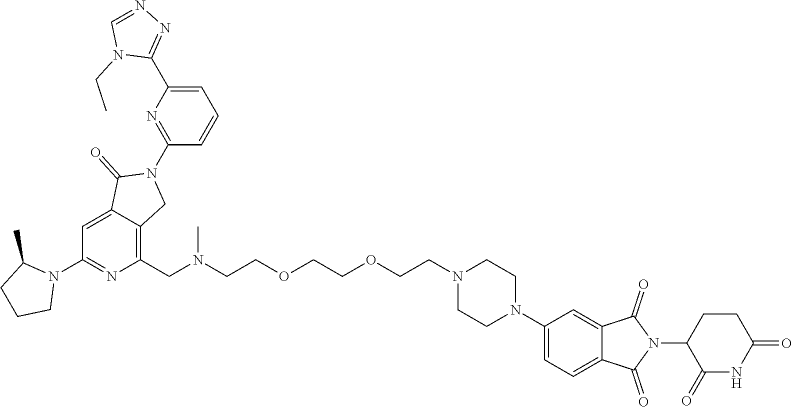 [SMILES: CCn1cnnc1-c1cccc(N2Cc3c(cc(N4CCC[C@H]4C)nc3CN(C)CCOCCOCCN3CCN(c4ccc5c(c4)C(=O)N(C4CCC(=O)NC4=O)C5=O)CC3)C2=O)n1] | 2-(2,6-dioxopiperidin-3-yl)-5-(4-(2- (2-(2-(((2-(6-(4-ethyl-4H-1,2,4- triazol-3-yl)pyridin-2-yl)-6-((R)-2- methylpyrrolidin-1-yl)-1-oxo-2,3- dihydro-1H-pyrrolo[3,4-c]pyridin-4- yl)methyl)(methyl)amino)ethoxy) ethoxy)ethyl)piperazin-1-yl) isoindoline-1,3-dione |
| 69 | MS-6- 049 | 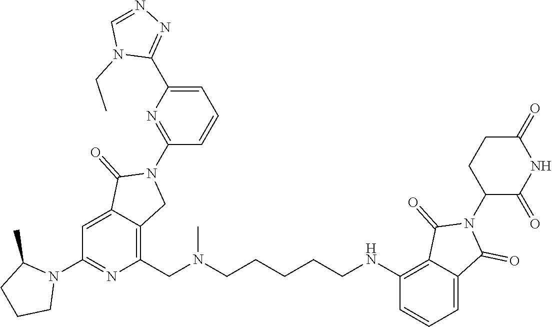 [SMILES: CCn1cnnc1-c1cccc(N2Cc3c(cc(N4CCC[C@H]4C)nc3CN(C)CCCCCNc3cccc4c3C(=O)N(C3CCC(=O)NC3=O)C4=O)C2=O)n1] | 2-(2,6-dioxopiperidin-3-yl)-4-((5- (((2-(6-(4-ethyl-4H-1,2,4-triazol-3- yl)pyridin-2-yl)-6-((R)-2- methylpyrrolidin-1-yl)-1-oxo-2,3- dihydro-1H-pyrrolo[3,4-c]pyridin-4- yl)methyl)(methyl)amino)pentyl) amino)isoindoline-1,3-dione |
| 70 | MS-6- 050 | 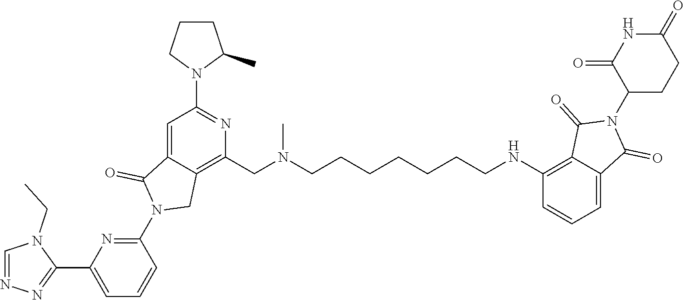 [SMILES: CCn1cnnc1-c1cccc(N2Cc3c(cc(N4CCC[C@H]4C)nc3CN(C)CCCCCCCNc3cccc4c3C(=O)N(C3CCC(=O)NC3=O)C4=O)C2=O)n1] | 2-(2,6-dioxopiperidin-3-yl)-4-((7- (((2-(6-(4-ethyl-4H-1,2,4-triazol-3- yl)pyridin-2-yl)-6-((R)-2- methylpyrrolidin-1-yl)-1-oxo-2,3- dihydro-1H-pyrrolo[3,4-c]pyridin-4- yl)methyl)(methyl)amino)heptyl) amino)isoindoline-1,3-dione |
| 71 | MS-6- 051 | 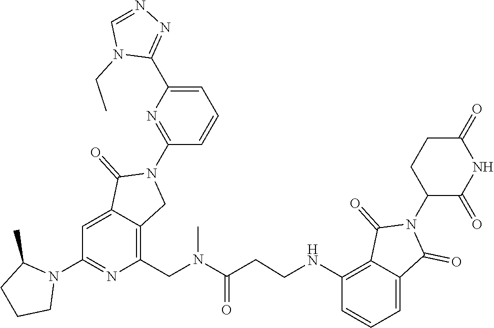 [SMILES: CCn1cnnc1-c1cccc(N2Cc3c(cc(N4CCC[C@H]4C)nc3CN(C)C(=O)CCNc3cccc4c3C(=O)N(C3CCC(=O)NC3=O)C4=O)C2=O)n1] | 3-((2-(2,6-dioxopiperidin-3-yl)-1,3- dioxoisoindolin-4-yl)amino)-N-((2- (6-(4-ethyl-4H-1,2,4-triazol-3- yl)pyridin-2-yl)-6-((R)-2- methylpyrrolidin-1-yl)-1-oxo-2,3- dihydro-1H-pyrrolo[3,4-c]pyridin-4- yl)methyl)-N-methylpropanamide |
| 72 | MS-6- 052 | 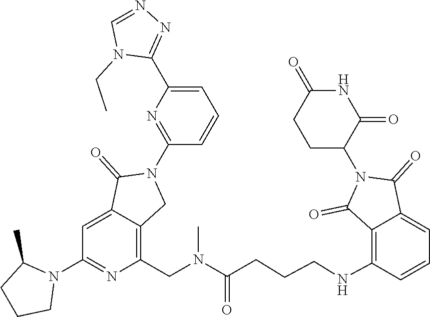 [SMILES: CCn1cnnc1-c1cccc(N2Cc3c(cc(N4CCC[C@H]4C)nc3CN(C)C(=O)CCCNc3cccc4c3C(=O)N(C3CCC(=O)NC3=O)C4=O)C2=O)n1] | 4-((2-(2,6-dioxopiperidin-3-yl)-1,3- dioxoisoindolin-4-yl)amino)-N-((2- (6-(4-ethyl-4H-1,2,4-triazol-3- yl)pyridin-2-yl)-6-((R)-2- methylpyrrolidin-1-yl)-1-oxo-2,3- dihydro-1H-pyrrolo[3,4-c]pyridin-4- yl)methyl)-N-methylbutanamide |
| 73 | MS-6- 053 | 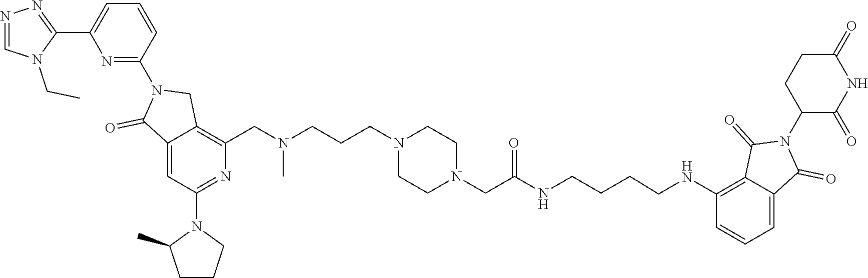 [SMILES: CCn1cnnc1-c1cccc(N2Cc3c(cc(N4CCC[C@H]4C)nc3CN(C)CCCN3CCN(CC(=O)NCCCCNc4cccc5c4C(=O)N(C4CCC(=O)NC4=O)C5=O)CC3)C2=O)n1] | N-(4-((2-(2,6-dioxopiperidin-3-yl)- 1,3-dioxoisoindolin-4- yl)amino)butyl)-2-(4-(3- (((2-(6-(4-ethyl-4H-1,2,4- triazol-3-yl)pyridin-2-yl)-6-((R)- 2-methylpyrrolidin-1-yl)-1- oxo-2,3-dihydro-1H-pyrrolo[3,4- c]pyridin-4- yl)methyl)(methyl)amino)propyl) piperazin-1-yl)acetamide |
| 74 | MS-6- 054 | 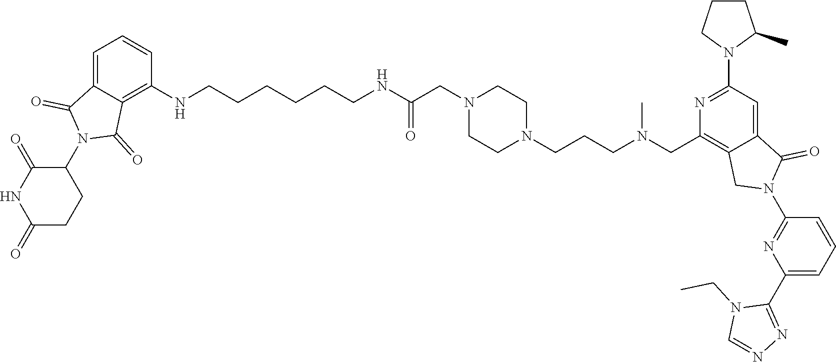 [SMILES: CCn1cnnc1-c1cccc(N2Cc3c(cc(N4CCC[C@H]4C)nc3CN(C)CCCN3CCN(CC(=O)NCCCCCCNc4cccc5c4C(=O)N(C4CCC(=O)NC4=O)C5=O)CC3)C2=O)n1] | N-(6-((2-(2,6-dioxopiperidin-3-yl)- 1,3-dioxoisoindolin-4- yl)amino)hexyl)-2-(4-(3- (((2-(6-(4-ethyl-4H-1,2,4-triazol- 3-yl)pyridin-2-yl)-6-((R)-2- methylpyrrolidin-1-yl)-1- oxo-2,3-dihydro-1H-pyrrolo[3,4- c]pyridin-4- yl)methyl)(methyl)amino)propyl) piperazin-1-yl)acetamide |
| 75 | MS-6- 055 | 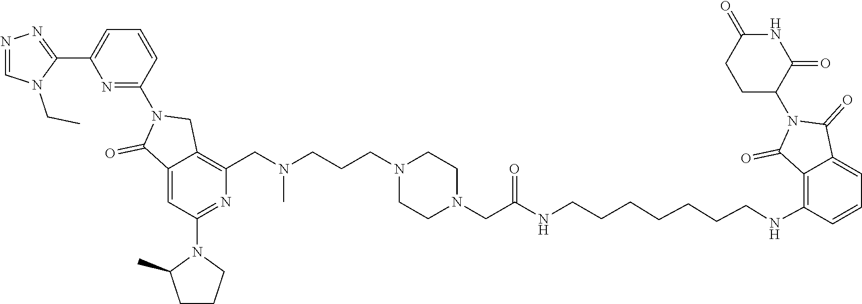 [SMILES: CCn1cnnc1-c1cccc(N2Cc3c(cc(N4CCC[C@H]4C)nc3CN(C)CCCN3CCN(CC(=O)NCCCCCCCNc4cccc5c4C(=O)N(C4CCC(=O)NC4=O)C5=O)CC3)C2=O)n1] | N-(7-((2-(2,6-dioxopiperidin-3-yl)- 1,3-dioxoisoindolin-4- yl)amino)heptyl)-2-(4-(3- (((2-(6-(4-ethyl-4H-1,2,4- triazol-3-yl)pyridin-2-yl)-6-((R)- 2-methylpyrrolidin-1-yl)-1- oxo-2,3-dihydro-1H-pyrrolo[3,4- c]pyridin-4- yl)methyl)(methyl)amino)propyl) piperazin-1-yl)acetamide |
| 76 | MS-6- 058 | 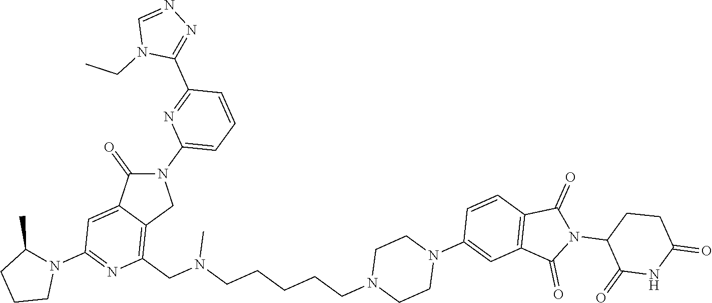 [SMILES: CCn1cnnc1-c1cccc(N2Cc3c(cc(N4CCC[C@H]4C)nc3CN(C)CCCCCN3CCN(c4ccc5c(c4)C(=O)N(C4CCC(=O)NC4=O)C5=O)CC3)C2=O)n1] | 2-(2,6-dioxopiperidin-3-yl)-5-(4-(5- (((2-(6-(4-ethyl-4H-1,2,4-triazol-3- yl)pyridin-2-yl)-6-((R)-2- methylpyrrolidin-1-yl)-1-oxo-2,3- dihydro-1H-pyrrolo[3,4-c]pyridin-4- yl)methyl)(methyl)amino)pentyl) piperazin-1-yl)isoindoline-1,3-dione |
| 77 | MS-6- 059 | 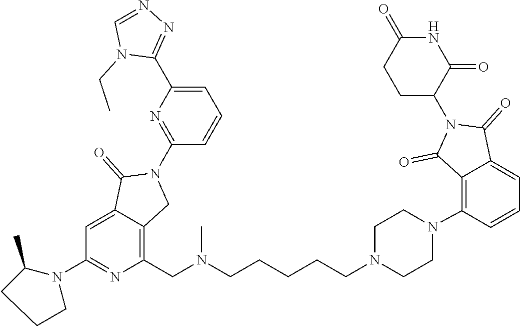 [SMILES: CCn1cnnc1-c1cccc(N2Cc3c(cc(N4CCC[C@H]4C)nc3CN(C)CCCCCN3CCN(c4cccc5c4C(=O)N(C4CCC(=O)NC4=O)C5=O)CC3)C2=O)n1] | 2-(2,6-dioxopiperidin-3-yl)-4-(4-(5- (((2-(6-(4-ethyl-4H-1,2,4-triazol-3- yl)pyridin-2-yl)-6-((R)-2- methylpyrrolidin-1-yl)-1-oxo-2,3- dihydro-1H-pyrrolo[3,4-c]pyridin-4- yl)methyl)(methyl)amino)pentyl) piperazin-1-yl)isoindoline-1,3-dione |
| 78 | MS-6- 060 | 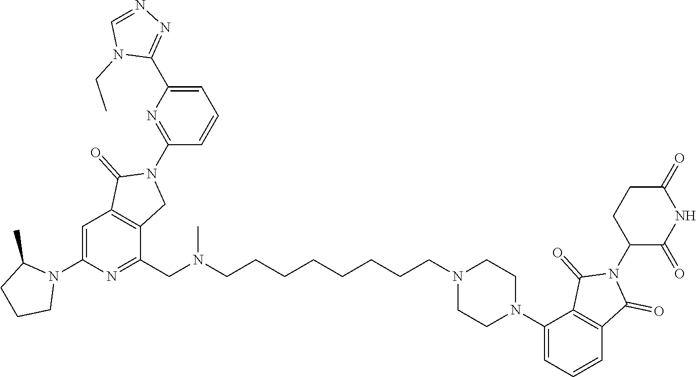 [SMILES: CCn1cnnc1-c1cccc(N2Cc3c(cc(N4CCC[C@H]4C)nc3CN(C)CCCCCCCCN3CCN(c4cccc5c4C(=O)N(C4CCC(=O)NC4=O)C5=O)CC3)C2=O)n1] | 2-(2,6-dioxopiperidin-3-yl)-4-(4-(8- (((2-(6-(4-ethyl-4H-1,2,4-triazol-3- yl)pyridin-2-yl)-6-((R)-2- methylpyrrolidin-1-yl)-1-oxo-2,3- dihydro-1H-pyrrolo[3,4-c]pyridin-4- yl)methyl)(methyl)amino)octyl) piperazin-1-yl)isoindoline-1,3-dione |
| 79 | MS-6- 061 | 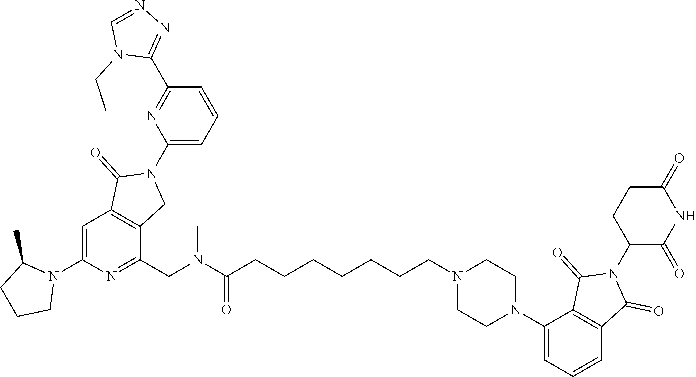 [SMILES: CCn1cnnc1-c1cccc(N2Cc3c(cc(N4CCC[C@H]4C)nc3CN(C)C(=O)CCCCCCCN3CCN(c4cccc5c4C(=O)N(C4CCC(=O)NC4=O)C5=O)CC3)C2=O)n1] | 8-(4-(2-(2,6-dioxopiperidin-3-yl)- 1,3-dioxoisoindolin-4-yl)piperazin- 1-yl)-N-((2-(6-(4-ethyl-4H-1,2,4- triazol-3-yl)pyridin-2-yl)-6-((R)-2- methylpyrrolidin-1-yl)-1-oxo-2,3- dihydro-1H-pyrrolo[3,4-c]pyridin-4- yl)methyl)-N-methyloctanamide |

TABLE 2

| Compound Structure | Compound Name |
|---|---|
| 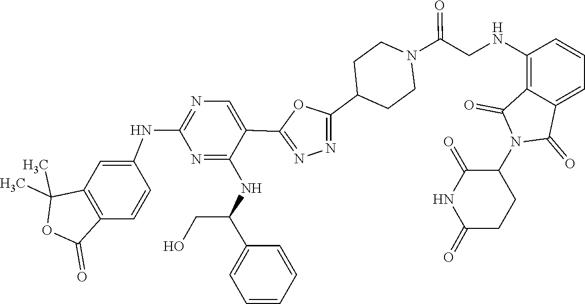 [SMILES: CC1(C)OC(=O)c2ccc(Nc3ncc(-c4nnc(C5CCN(C(=O)CNc6cccc7c6C(=O)N(C6CCC(=O)NC6=O)C7=O)CC5)o4)c(N[C@H](CO)c4ccccc4)n3)cc21] | 4-((2-(4-(5-(2-((3,3-dimethyl-1-oxo-1,3- dihydroisobenzofuran-5-yl)amino)-4- (((S)-2-hydroxy-1- phenylethyl)amino)pyrimidin-5-yl)- 1,3,4-oxadiazol-2-yl)piperidin-1-yl)-2- oxoethyl)amino)-2-(2,6-dioxopiperidin- 3-yl)isoindoline-1,3-dione |
| 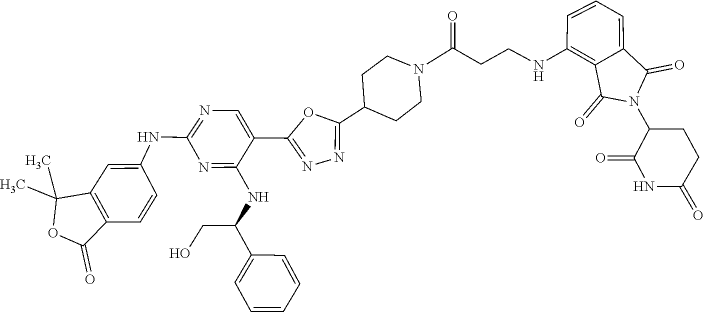 [SMILES: CC1(C)OC(=O)c2ccc(Nc3ncc(-c4nnc(C5CCN(C(=O)CCNc6cccc7c6C(=O)N(C6CCC(=O)NC6=O)C7=O)CC5)o4)c(N[C@H](CO)c4ccccc4)n3)cc21] | 4-((3-(4-(5-(2-((3,3-dimethyl-1-oxo-1,3- dihydroisobenzofuran-5-yl)amino)-4-(((S)- 2-hydroxy-1-phenylethyl)amino)pyrimidin-5-yl)- 1,3,4-oxadiazol-2-yl)piperidin-1-yl)-3- oxopropyl)amino)-2-(2,6- dioxopiperidin-3-yl)isoindoline-1,3-dione |
| 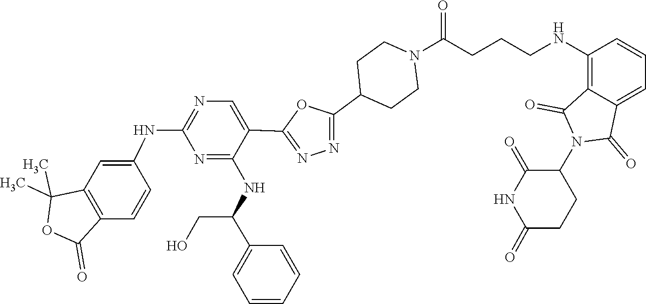 [SMILES: CC1(C)OC(=O)c2ccc(Nc3ncc(-c4nnc(C5CCN(C(=O)CCCNc6cccc7c6C(=O)N(C6CCC(=O)NC6=O)C7=O)CC5)o4)c(N[C@H](CO)c4ccccc4)n3)cc21] | 4-((4-(4-(5-(2-((3,3-dimethyl-1-oxo-1,3- dihydroisobenzofuran-5-yl)amino)-4- (((S)-2-hydroxy-1- phenylethyl)amino)pyrimidin-5-yl)- 1,3,4-oxadiazol-2-yl)piperidin-1-yl)-4- oxobutyl)amino)-2-(2,6-dioxopiperidin- 3-yl)isoindoline-1,3-dione |
| 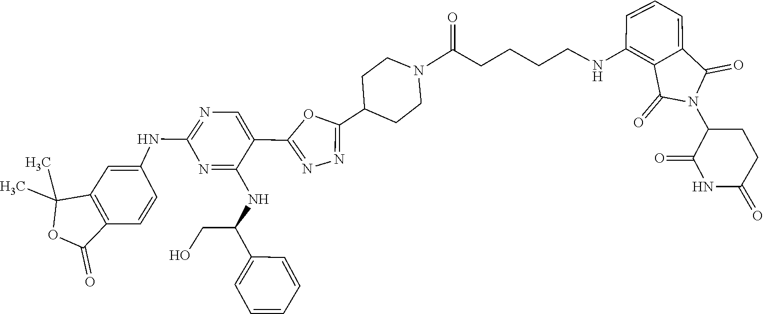 [SMILES: CC1(C)OC(=O)c2ccc(Nc3ncc(-c4nnc(C5CCN(C(=O)CCCCNc6cccc7c6C(=O)N(C6CCC(=O)NC6=O)C7=O)CC5)o4)c(N[C@H](CO)c4ccccc4)n3)cc21] | 4-((5-(4-(5-(2-((3,3-dimethyl-1-oxo-1,3- dihydroisobenzofuran-5-yl)amino)-4- (((S)-2-hydroxy-1- phenylethyl)amino)pyrimidin-5-yl)- 1,3,4-oxadiazol-2-yl)piperidin-1-yl)-5- oxopentyl)amino)-2-(2,6- dioxopiperidin-3-yl)isoindoline-1,3-dione |
| 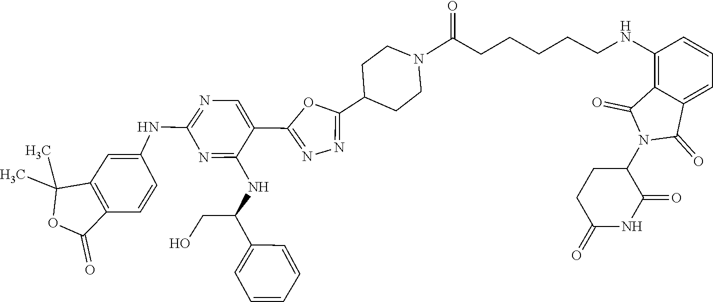 [SMILES: CC1(C)OC(=O)c2ccc(Nc3ncc(-c4nnc(C5CCN(C(=O)CCCCCNc6cccc7c6C(=O)N(C6CCC(=O)NC6=O)C7=O)CC5)o4)c(N[C@H](CO)c4ccccc4)n3)cc21] | 4-((6-(4-(5-(2-((3,3-dimethyl-1-oxo-1,3- dihydroisobenzofuran-5-yl)amino)-4- (((S)-2-hydroxy-1- phenylethyl)amino)pyrimidin-5-yl)- 1,3,4-oxadiazol-2-yl)piperidin-1-yl)-6- oxohexyl)amino)-2-(2,6-dioxopiperidin- 3-yl)isoindoline-1,3-dione |
| 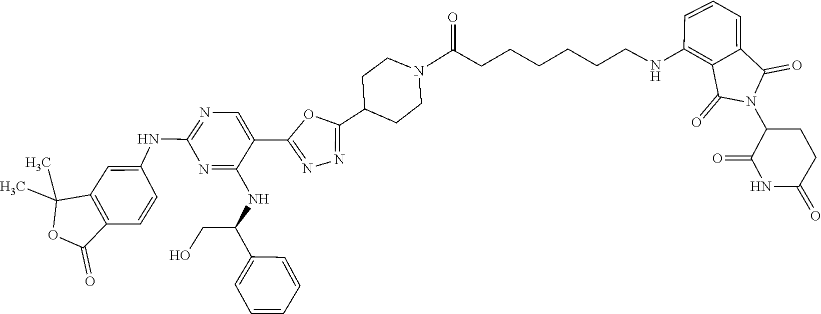 [SMILES: CC1(C)OC(=O)c2ccc(Nc3ncc(-c4nnc(C5CCN(C(=O)CCCCCCNc6cccc7c6C(=O)N(C6CCC(=O)NC6=O)C7=O)CC5)o4)c(N[C@H](CO)c4ccccc4)n3)cc21] | 4-((7-(4-(5-(2-((3,3-dimethyl-1-oxo-1,3- dihydroisobenzofuran-5-yl)amino)-4- (((S)-2-hydroxy-1- phenylethyl)amino)pyrimidin-5-yl)- 1,3,4-oxadiazol-2-yl)piperidin-1-yl)-7- oxoheptyl)amino)-2-(2,6- dioxopiperidin-3-yl)isoindoline-1,3-dione |
| 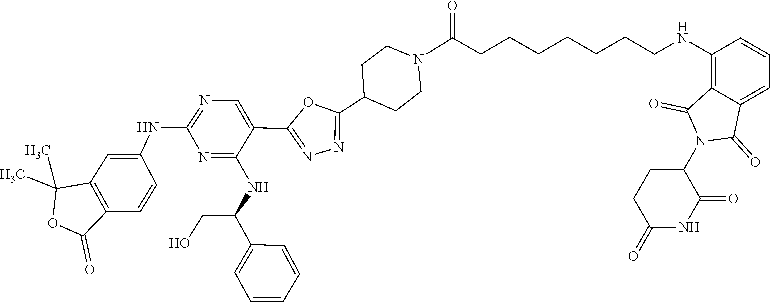 [SMILES: CC1(C)OC(=O)c2ccc(Nc3ncc(-c4nnc(C5CCN(C(=O)CCCCCCCNc6cccc7c6C(=O)N(C6CCC(=O)NC6=O)C7=O)CC5)o4)c(N[C@H](CO)c4ccccc4)n3)cc21] | 4-((8-(4-(5-(2-((3,3-dimethyl-1-oxo-1,3- dihydroisobenzofuran-5-yl)amino)-4- (((S)-2-hydroxy-1- phenylethyl)amino)pyrimidin-5-yl)- 1,3,4-oxadiazol-2-yl)piperidin-1-yl)-8- oxooctyl)amino)-2-(2,6-dioxopiperidin- 3-yl)isoindoline-1,3-dione |
| 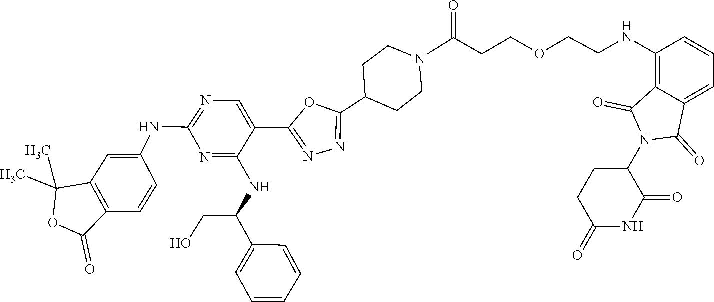 [SMILES: CC1(C)OC(=O)c2ccc(Nc3ncc(-c4nnc(C5CCN(C(=O)CCOCCNc6cccc7c6C(=O)N(C6CCC(=O)NC6=O)C7=O)CC5)o4)c(N[C@H](CO)c4ccccc4)n3)cc21] | 4-((2-(3-(4-(5-(2-((3,3-dimethyl-1-oxo- 1,3-dihydroisobenzofuran-5-yl)amino)- 4-(((S)-2-hydroxy-1- phenylethyl)amino)pyrimidin-5-yl)- 1,3,4-oxadiazol-2-yl)piperidin-1-yl)-3- oxopropoxy)ethyl)amino)-2-(2,6- dioxopiperidin-3-yl)isoindoline-1,3-dione |
| 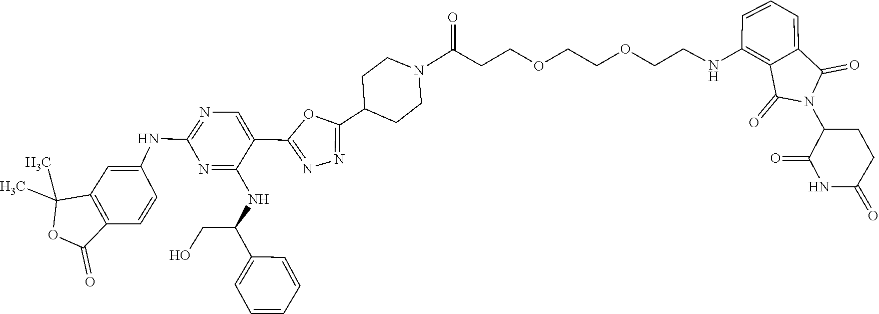 [SMILES: CC1(C)OC(=O)c2ccc(Nc3ncc(-c4nnc(C5CCN(C(=O)CCOCCOCCNc6cccc7c6C(=O)N(C6CCC(=O)NC6=O)C7=O)CC5)o4)c(N[C@H](CO)c4ccccc4)n3)cc21] | 4-((2-(2-(3-(4-(5-(2-((3,3-dimethyl-1-oxo-1,3- dihydroisobenzofuran-5-yl)amino)-4-(((S)-2- hydroxy-1-phenylethyl)amino)pyrimidin-5-yl)- 1,3,4-oxadiazol-2-yl)piperidin-1-yl)-3- oxopropoxy)ethoxy)ethyl)amino)-2- (2,6-dioxopiperidin-3-yl)isoindoline-1,3- dione |
| 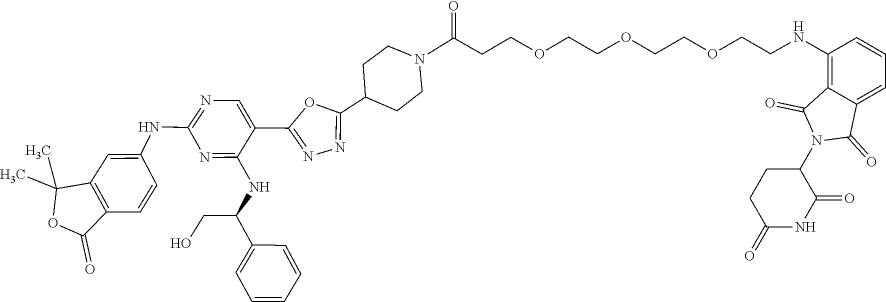 [SMILES: CC1(C)OC(=O)c2ccc(Nc3ncc(-c4nnc(C5CCN(C(=O)CCOCCOCCOCCNc6cccc7c6C(=O)N(C6CCC(=O)NC6=O)C7=O)CC5)o4)c(N[C@H](CO)c4ccccc4)n3)cc21] | 4-((2-(2-(2-(3-(4-(5-(2-((3,3-dimethyl-1- oxo-1,3-dihydroisobenzofuran-5- yl)amino)-4-(((S)-2-hydroxy-1- phenylethyl)amino)pyrimidin-5-yl)- 1,3,4-oxadiazol-2-yl)piperidin-1-yl)-3- oxopropoxy)ethoxy)ethoxy)ethyl)amino)-2- (2,6-dioxopiperidin-3-yl)isoindoline-1,3-dione |
| 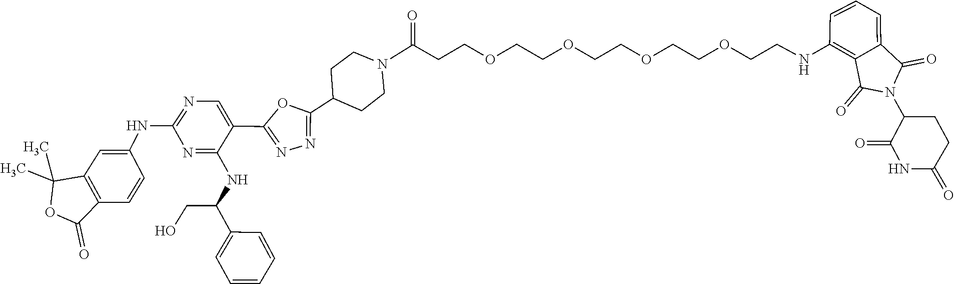 [SMILES: CC1(C)OC(=O)c2ccc(Nc3ncc(-c4nnc(C5CCN(C(=O)CCOCCOCCOCCOCCNc6cccc7c6C(=O)N(C6CCC(=O)NC6=O)C7=O)CC5)o4)c(N[C@H](CO)c4ccccc4)n3)cc21] | 4-((15-(4-(5-(2-((3,3-dimethyl-1-oxo- 1,3-dihydroisobenzofuran-5-yl)amino)- 4-(((S)-2-hydroxy-1- phenylethyl)amino)pyrimidin-5-yl)- 1,3,4-oxadiazol-2-yl)piperidin-1-yl)-15- oxo-3,6,9,12- tetraoxapentadecyl)amino)-2-(2,6- dioxopiperidin-3-yl)isoindoline-1,3-dione |
| 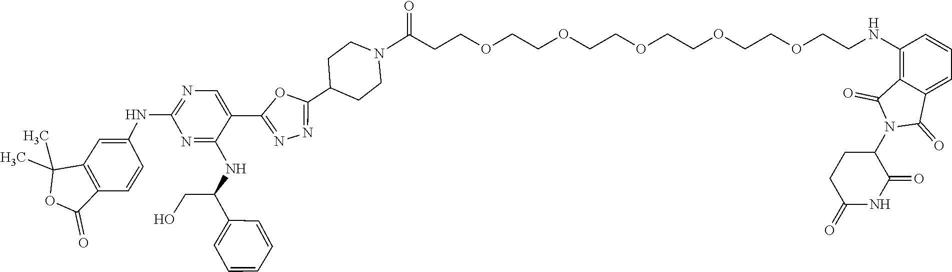 [SMILES: CC1(C)OC(=O)c2ccc(Nc3ncc(-c4nnc(C5CCN(C(=O)CCOCCOCCOCCOCCOCCNc6cccc7c6C(=O)N(C6CCC(=O)NC6=O)C7=O)CC5)o4)c(N[C@H](CO)c4ccccc4)n3)cc21] | 4-((18-(4-(5-(2-((3,3-dimethyl-1-oxo- 1,3-dihydroisobenzofuran-5-yl)amino)- 4-(((S)-2-hydroxy-1- phenylethyl)amino)pyrimidin-5-yl)- 1,3,4-oxadiazol-2-yl)piperidin-1-yl)-18- oxo-3,6,9,12,15- pentaoxaoctadecyl)amino)-2-(2,6- dioxopiperidin-3-yl)isoindoline-1,3- dione |
| 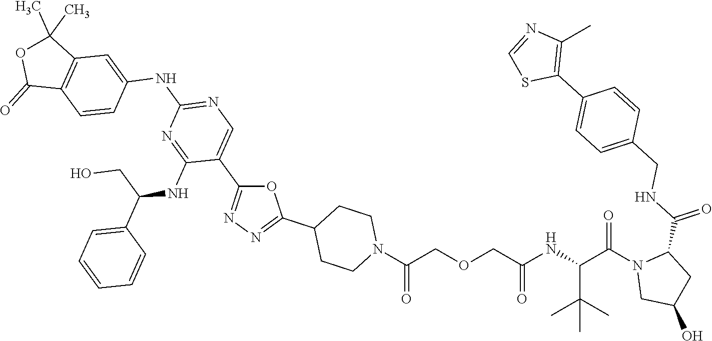 [SMILES: Cc1ncsc1-c1ccc(CNC(=O)[C@@H]2C[C@@H](O)CN2C(=O)[C@@H](NC(=O)COCC(=O)N2CCC(c3nnc(-c4cnc(Nc5ccc6c(c5)C(C)(C)OC6=O)nc4N[C@H](CO)c4ccccc4)o3)CC2)C(C)(C)C)cc1] | (2S,4R)-1-((S)-2-(2-(2-(4-(5-(2-((3,3- dimethyl-1-oxo-1,3- dihydroisobenzofuran-5-yl)amino)-4- (((S)-2-hydroxy-1- phenylethyl)amino)pyrimidin-5-yl)- 1,3,4-oxadiazol-2-yl)piperidin-1-yl)-2- oxoethoxy)acetamido)-3,3- dimethylbutanoyl)-4-hydroxy-N-(4-(4- methylthiazol-5-yl)benzyl)pyrrolidine-2- carboxamide |
| 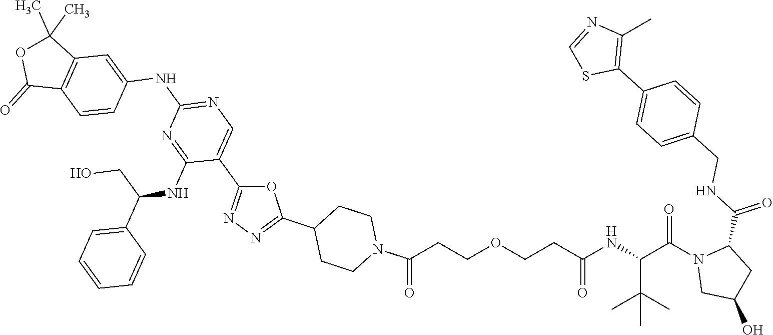 [SMILES: Cc1ncsc1-c1ccc(CNC(=O)[C@@H]2C[C@@H](O)CN2C(=O)[C@@H](NC(=O)CCOCCC(=O)N2CCC(c3nnc(-c4cnc(Nc5ccc6c(c5)C(C)(C)OC6=O)nc4N[C@H](CO)c4ccccc4)o3)CC2)C(C)(C)C)cc1] | (2S,4R)-1-((S)-2-(3-(3-(4-(5-(2-((3,3- dimethyl-1-oxo-1,3- dihydroisobenzofuran-5-yl)amino)-4- (((S)-2-hydroxy-1- phenylethyl)amino)pyrimidin-5-yl)- 1,3,4-oxadiazol-2-yl)piperidin-1-yl)-3- oxopropoxy)propanamido)-3,3- dimethylbutanoyl)-4-hydroxy-N-(4-(4- methylthiazol-5-yl)benzyl)pyrrolidine-2- carboxamide |
| 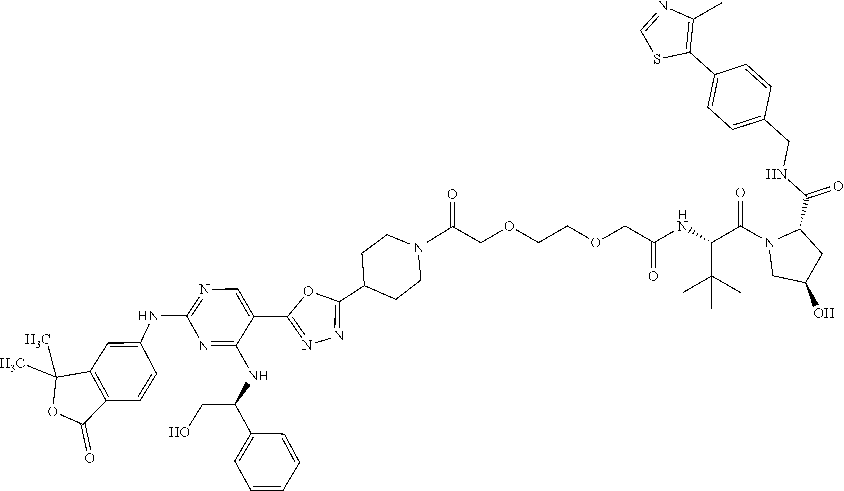 [SMILES: Cc1ncsc1-c1ccc(CNC(=O)[C@@H]2C[C@@H](O)CN2C(=O)[C@@H](NC(=O)COCCOCC(=O)N2CCC(c3nnc(-c4cnc(Nc5ccc6c(c5)C(C)(C)OC6=O)nc4N[C@H](CO)c4ccccc4)o3)CC2)C(C)(C)C)cc1] | (2S,4R)-1-((S)-2-(2-(2-(2-(4-(5-(2-((3,3- dimethyl-1-oxo-1,3- dihydroisobenzofuran-5-yl)amino)-4- (((S)-2-hydroxy-1- phenylethyl)amino)pyrimidin-5-yl)- 1,3,4-oxadiazol-2-yl)piperidin-1-yl)-2- oxoethoxy)ethoxy)acetamido)-3,3- dimethylbutanoyl)-4-hydroxy-N-(4-(4- methylthiazol-5-yl)benzyl)pyrrolidine-2- carboxamide |
| 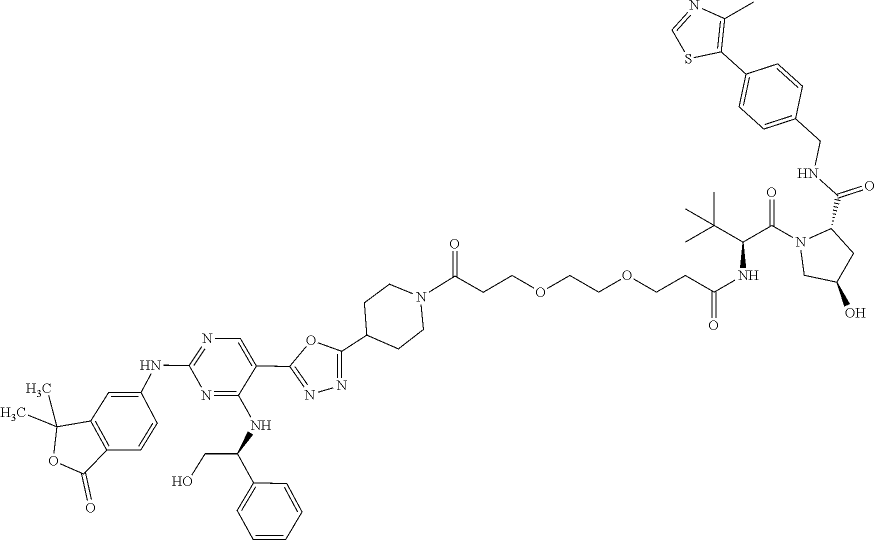 [SMILES: Cc1ncsc1-c1ccc(CNC(=O)[C@@H]2C[C@@H](O)CN2C(=O)[C@@H](NC(=O)CCOCCOCCC(=O)N2CCC(c3nnc(-c4cnc(Nc5ccc6c(c5)C(C)(C)OC6=O)nc4N[C@H](CO)c4ccccc4)o3)CC2)C(C)(C)C)cc1] | (2S,4R)-1-((S)-2-(3-(2-(3-(4-(5-(2-((3,3- dimethyl-1-oxo-1,3- dihydroisobenzofuran-5-yl)amino)-4- (((S)-2-hydroxy-1- phenylethyl)amino)pyrimidin-5-yl)- 1,3,4-oxadiazol-2-yl)piperidin-1-yl)-3- oxopropoxy)ethoxy)propanamido)-3,3- dimethylbutanoyl)-4-hydroxy-N-(4-(4- methylthiazol-5-yl)benzyl)pyrrolidine-2- carboxamide |
| 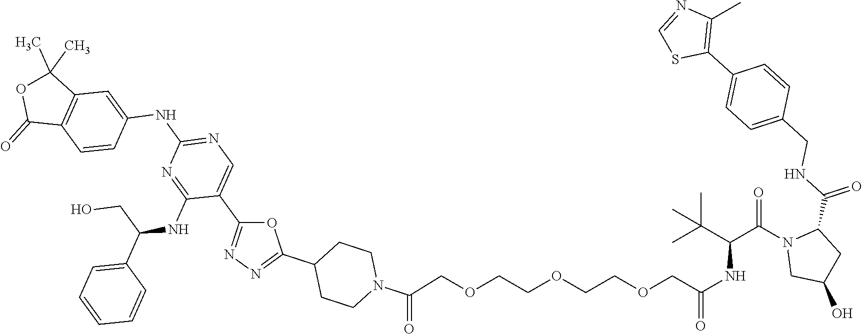 [SMILES: Cc1ncsc1-c1ccc(CNC(=O)[C@@H]2C[C@@H](O)CN2C(=O)[C@@H](NC(=O)COCCOCCOCC(=O)N2CCC(c3nnc(-c4cnc(Nc5ccc6c(c5)C(C)(C)OC6=O)nc4N[C@H](CO)c4ccccc4)o3)CC2)C(C)(C)C)cc1] | (2S,4R)-1-((S)-2-(tert-butyl)-14-(4-(5- (2-((3,3-dimethyl-1-oxo-1,3- dihydroisobenzofuran-5-yl)amino)-4- (((S)-2-hydroxy-1- phenylethyl)amino)pyrimidin-5-yl)- 1,3,4-oxadiazol-2-yl)piperidin-1-yl)- 4,14-dioxo-6,9,12-trioxa-3- azatetradecanoyl)-4-hydroxy-N-(4-(4- methylthiazol-5-yl)benzyl)pyrrolidine-2- carboxamide |
| 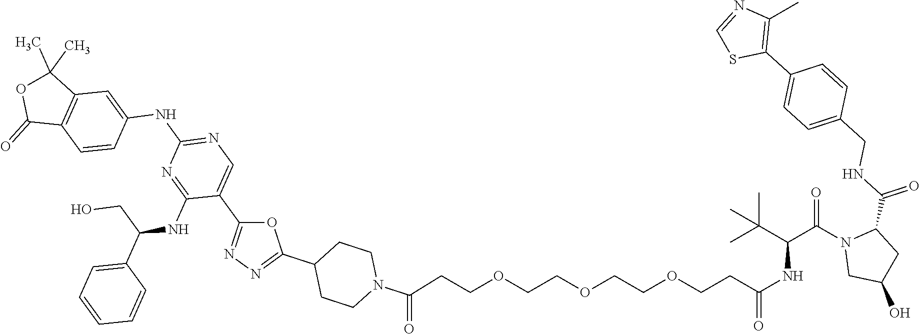 [SMILES: Cc1ncsc1-c1ccc(CNC(=O)[C@@H]2C[C@@H](O)CN2C(=O)[C@@H](NC(=O)CCOCCOCCOCCC(=O)N2CCC(c3nnc(-c4cnc(Nc5ccc6c(c5)C(C)(C)OC6=O)nc4N[C@H](CO)c4ccccc4)o3)CC2)C(C)(C)C)cc1] | (2S,4R)-1-((S)-2-(tert-butyl)-16-(4-(5- (2-((3,3-dimethyl-1-oxo-1,3- dihydroisobenzofuran-5-yl)amino)-4- (((S)-2-hydroxy-1- phenylethyl)amino)pyrimidin-5-yl)- 1,3,4-oxadiazol-2-yl)piperidin-1-yl)- 4,16-dioxo-7,10,13-trioxa-3- azahexadecanoyl)-4-hydroxy-N-(4-(4- methylthiazol-5-yl)benzyl)pyrrolidine-2- carboxamide |
| 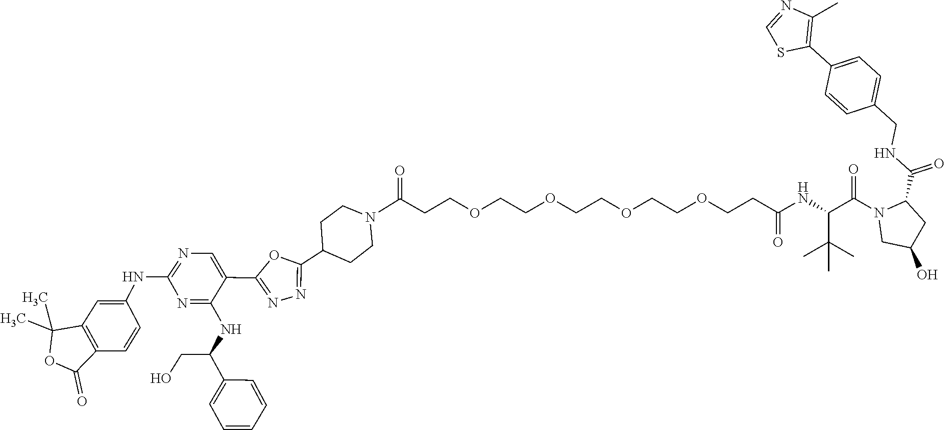 [SMILES: Cc1ncsc1-c1ccc(CNC(=O)[C@@H]2C[C@@H](O)CN2C(=O)[C@@H](NC(=O)CCOCCOCCOCCOCCC(=O)N2CCC(c3nnc(-c4cnc(Nc5ccc6c(c5)C(C)(C)OC6=O)nc4N[C@H](CO)c4ccccc4)o3)CC2)C(C)(C)C)cc1] | (2S,4R)-1-((S)-2-(tert-butyl)-19-(4-(5- (2-((3,3-dimethyl-1-oxo-1,3- dihydroisobenzofuran-5-yl)amino)-4- (((S)-2-hydroxy-1- phenylethyl)amino)pyrimidin-5-yl)- 1,3,4-oxadiazol-2-yl)piperidin-1-yl)- 4,19-dioxo-7,10,13,16-tetraoxa-3- azanonadecanoyl)-4-hydroxy-N-(4-(4- methylthiazol-5-yl)benzyl)pyrrolidine-2- carboxamide |
| 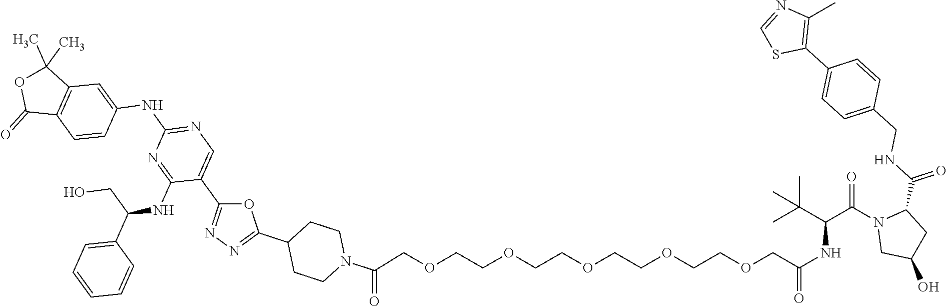 [SMILES: Cc1ncsc1-c1ccc(CNC(=O)[C@@H]2C[C@@H](O)CN2C(=O)[C@@H](NC(=O)COCCOCCOCCOCCOCC(=O)N2CCC(c3nnc(-c4cnc(Nc5ccc6c(c5)C(C)(C)OC6=O)nc4N[C@H](CO)c4ccccc4)o3)CC2)C(C)(C)C)cc1] | (2S,4R)-1-((S)-2-(tert-butyl)-20-(4-(5- (2-((3,3-dimethyl-1-oxo-1,3- dihydroisobenzofuran-5-yl)amino)-4- (((S)-2-hydroxy-1- phenylethyl)amino)pyrimidin-5-yl)- 1,3,4-oxadiazol-2-yl)piperidin-1-yl)- 4,20-dioxo-6,9,12,15,18-pentaoxa-3- azaicosanoyl)-4-hydroxy-N-(4-(4- methylthiazol-5-yl)benzyl)pyrrolidine-2- carboxamide |
| 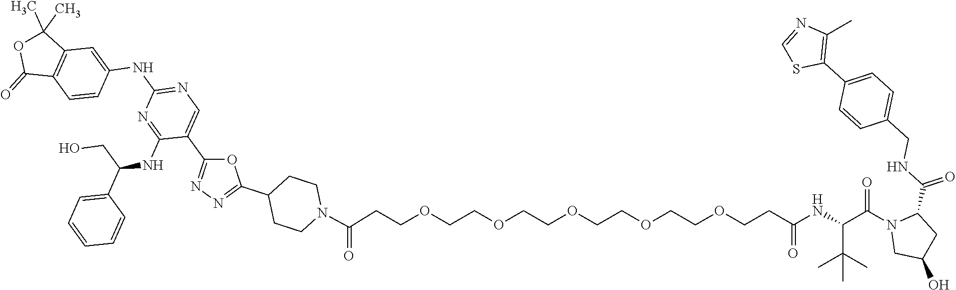 [SMILES: Cc1ncsc1-c1ccc(CNC(=O)[C@@H]2C[C@@H](O)CN2C(=O)[C@@H](NC(=O)CCOCCOCCOCCOCCOCCC(=O)N2CCC(c3nnc(-c4cnc(Nc5ccc6c(c5)C(C)(C)OC6=O)nc4N[C@H](CO)c4ccccc4)o3)CC2)C(C)(C)C)cc1] | (2S,4R)-1-((S)-2-(tert-butyl)-22-(4-(5- (2-((3,3-dimethyl-1-oxo-1,3- dihydoisobenzofuran-5-yl)amino)-4- (((S)-2-hydroxy-1- phenylethyl)amino)pyrimidin-5-yl)- 1,3,4-oxadiazol-2-yl)piperidin-1-yl)- 4,22-dioxo-7,10,13,16,19-pentaoxa-3- azadocosanoyl)-4-hydroxy-N-(4-(4- methylthiazol-5-yl)benzyl)pyrrolidine-2- carboxamide |
| 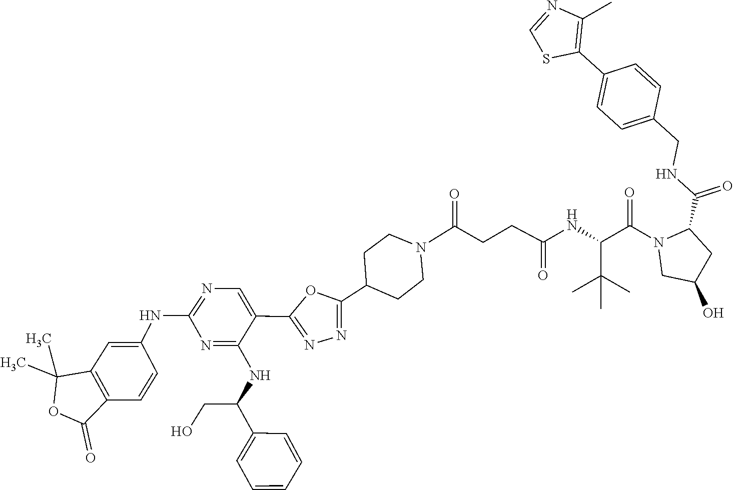 [SMILES: Cc1ncsc1-c1ccc(CNC(=O)[C@@H]2C[C@@H](O)CN2C(=O)[C@@H](NC(=O)CCC(=O)N2CCC(c3nnc(-c4cnc(Nc5ccc6c(c5)C(C)(C)OC6=O)nc4N[C@H](CO)c4ccccc4)o3)CC2)C(C)(C)C)cc1] | (2S,4R)-1-((S)-2-(4-(4-(5-(2-((3,3- dimethyl-1-oxo-1,3- dihydroisobenzofuran-5-yl)amino)-4- (((S)-2-hydroxy-1- phenylethyl)amino)pyrimidin-5-yl)- 1,3,4-oxadiazol-2-yl)piperidin-1-yl)-4- oxobutanamido)-3,3- dimethylbutanoyl)-4-hydroxy-N-(4-(4- methylthiazol-5-yl)benzyl)pyrrolidine-2- carboxamide |
| 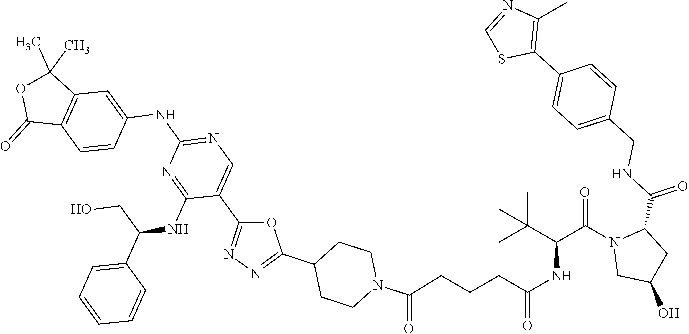 [SMILES: Cc1ncsc1-c1ccc(CNC(=O)[C@@H]2C[C@@H](O)CN2C(=O)[C@@H](NC(=O)CCCC(=O)N2CCC(c3nnc(-c4cnc(Nc5ccc6c(c5)C(C)(C)OC6=O)nc4N[C@H](CO)c4ccccc4)o3)CC2)C(C)(C)C)cc1] | (2S,4R)-1-((S)-2-(5-(4-(5-(2-((3,3- dimethyl-1-oxo-1,3- dihydroisobenzofuran-5-yl)amino)-4- (((S)-2-hydroxy-1- phenylethyl)amino)pyrimidin-5-yl)- 1,3,4-oxadiazol-2-yl)piperidin-1-yl)-5- oxopentanamido)-3,3- dimethylbutanoyl)-4-hydroxy-N-(4-(4- methylthiazol-5-yl)benzyl)pyrrolidine-2- carboxamide |
| 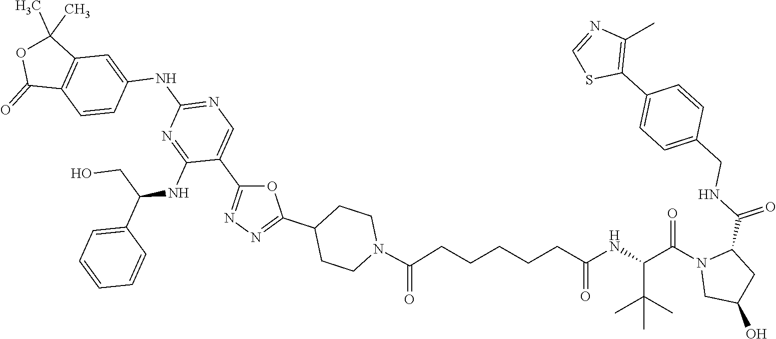 [SMILES: Cc1ncsc1-c1ccc(CNC(=O)[C@@H]2C[C@@H](O)CN2C(=O)[C@@H](NC(=O)CCCCCC(=O)N2CCC(c3nnc(-c4cnc(Nc5ccc6c(c5)C(C)(C)OC6=O)nc4N[C@H](CO)c4ccccc4)o3)CC2)C(C)(C)C)cc1] | (2S,4R)-1-((S)-2-(7-(4-(5-(2-((3,3- dimethyl-1-oxo-1,3- dihydroisobenzofuran-5-yl)amino)-4- (((S)-2-hydroxy-1- phenylethyl)amino)pyrimidin-5-yl)- 1,3,4-oxadiazol-2-yl)piperidin-1-yl)-7- oxoheptanamido)-3,3- dimethylbutanoyl)-4-hydroxy-N-(4-(4- methylthiazol-5-yl)benzyl)pyrrolidine-2- carboxamide |
| 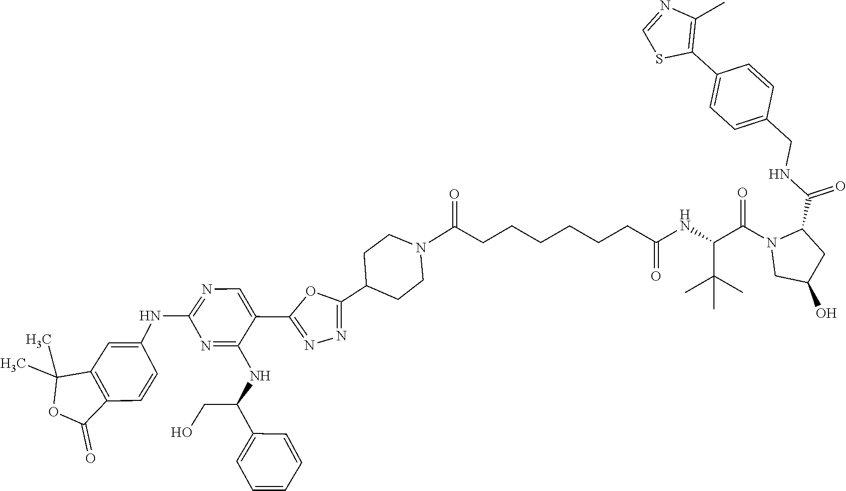 [SMILES: Cc1ncsc1-c1ccc(CNC(=O)[C@@H]2C[C@@H](O)CN2C(=O)[C@@H](NC(=O)CCCCCCC(=O)N2CCC(c3nnc(-c4cnc(Nc5ccc6c(c5)C(C)(C)OC6=O)nc4N[C@H](CO)c4ccccc4)o3)CC2)C(C)(C)C)cc1] | (2S,4R)-1-((S)-2-(8-(4-(5-(2-((3,3- dimethyl-1-oxo-1,3- dihydroisobenzofuran-5-yl)amino)-4- (((S)-2-hydroxy-1- phenylethyl)amino)pyrimidin-5-yl)- 1,3,4-oxadiazol-2-yl)piperidin-1-yl)-8- oxooctanamido)-3,3- dimethylbutanoyl)-4-hydroxy-N-(4-(4- methylthiazol-5-yl)benzyl)pyrrolidine-2- carboxamide |
| 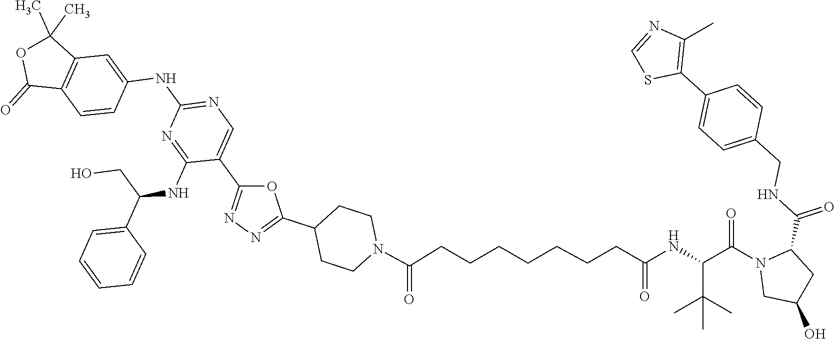 [SMILES: Cc1ncsc1-c1ccc(CNC(=O)[C@@H]2C[C@@H](O)CN2C(=O)[C@@H](NC(=O)CCCCCCCC(=O)N2CCC(c3nnc(-c4cnc(Nc5ccc6c(c5)C(C)(C)OC6=O)nc4N[C@H](CO)c4ccccc4)o3)CC2)C(C)(C)C)cc1] | (2S,4R)-1-((S)-2-(9-(4-(5-(2-((3,3- dimethyl-1-oxo-1,3- dihydroisobenzofuran-5-yl)amino)-4- (((S)-2-hydroxy-1- phenylethyl)amino)pyrimidin-5-yl)- 1,3,4-oxadiazol-2-yl)piperidin-1-yl)-9- oxononanamido)-3,3- dimethylbutanoyl)-4-hydroxy-N-(4-(4- methylthiazol-5-yl)benzyl)pyrrolidine-2- carboxamide |
| 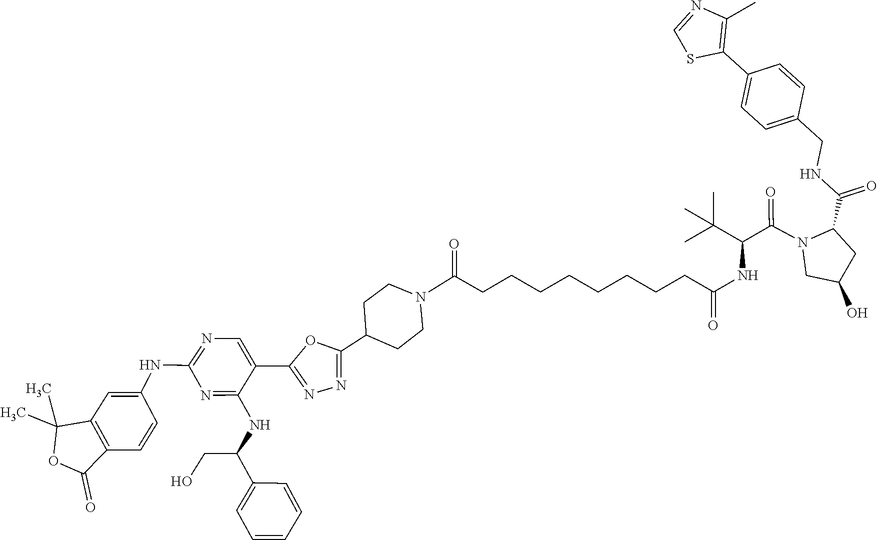 [SMILES: Cc1ncsc1-c1ccc(CNC(=O)[C@@H]2C[C@@H](O)CN2C(=O)[C@@H](NC(=O)CCCCCCCCC(=O)N2CCC(c3nnc(-c4cnc(Nc5ccc6c(c5)C(C)(C)OC6=O)nc4N[C@H](CO)c4ccccc4)o3)CC2)C(C)(C)C)cc1] | (2S,4R)-1-((S)-2-(10-(4-(5-(2-((3,3- dimethyl-1-oxo-1,3- dihydroisobenzofuran-5-yl)amino)-4- (((S)-2-hydroxy-1- phenylethyl)amino)pyrimidin-5-yl)- 1,3,4-oxadiazol-2-yl)piperidin-1-yl)-10- oxodecanamido)-3,3- dimethylbutanoyl)-4-hydroxy-N-(4-(4- methylthiazol-5-yl)benzyl)pyrrolidine-2- carboxamide |
| 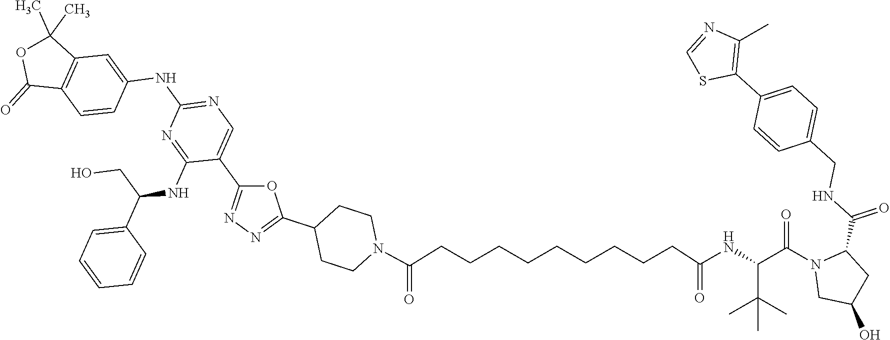 [SMILES: Cc1ncsc1-c1ccc(CNC(=O)[C@@H]2C[C@@H](O)CN2C(=O)[C@@H](NC(=O)CCCCCCCCCC(=O)N2CCC(c3nnc(-c4cnc(Nc5ccc6c(c5)C(C)(C)OC6=O)nc4N[C@H](CO)c4ccccc4)o3)CC2)C(C)(C)C)cc1] | (2S,4R)-1-((S)-2-(11-(4-(5-(2-((3,3- dimethyl-1-oxo-1,3- dihydroisobenzofuran-5-yl)amino)-4- (((S)-2-hydroxy-1- phenylethyl)amino)pyrimidin-5-yl)- 1,3,4-oxadiazol-2-yl)piperidin-1-yl)-11- oxoundecanamido)-3,3- dimethylbutanoyl)-4-hydroxy-N-(4-(4- methylthiazol-5-yl)benzyl)pyrrolidine-2- carboxamide |
| 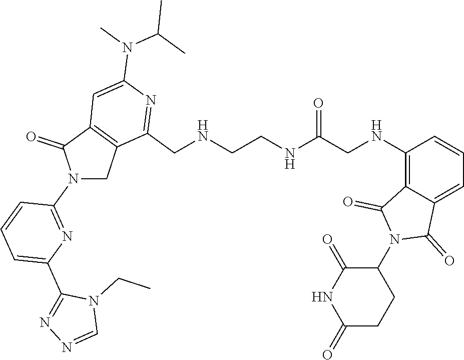 [SMILES: CCn1cnnc1-c1cccc(N2Cc3c(cc(N(C)C(C)C)nc3CNCCNC(=O)CNc3cccc4c3C(=O)N(C3CCC(=O)NC3=O)C4=O)C2=O)n1] | 2-((2-(2,6-dioxopiperidin-3-yl)-1,3- dioxoisoindolin-4-yl)amino)-N-(2-(((2- (6-(4-ethyl-4H-1,2,4-triazol-3-yl)pyridin- 2-yl)-6-(isopropyl(methyl)amino)-1- oxo-2,3-dihydro-1H-pyrrolo[3,4- c]pyridin-4- yl)methyl)amino)ethyl)acetamide |
| 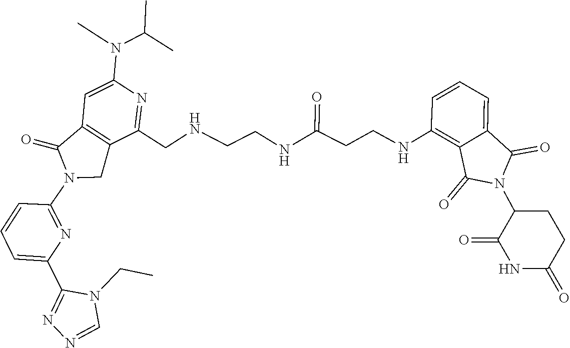 [SMILES: CCn1cnnc1-c1cccc(N2Cc3c(cc(N(C)C(C)C)nc3CNCCNC(=O)CCNc3cccc4c3C(=O)N(C3CCC(=O)NC3=O)C4=O)C2=O)n1] | 3-((2-(2,6-dioxopiperidin-3-yl)-1,3- dioxoisoindolin-4-yl)amino)-N-(2-(((2- (6-(4-ethyl-4H-1,2,4-triazol-3-yl)pyridin- 2-yl)-6-(isopropyl(methyl)amino)-1- oxo-2,3-dihydro-1H-pyrrolo[3,4- c]pyridin-4- yl)methyl)amino)ethyl)propanamide |
| 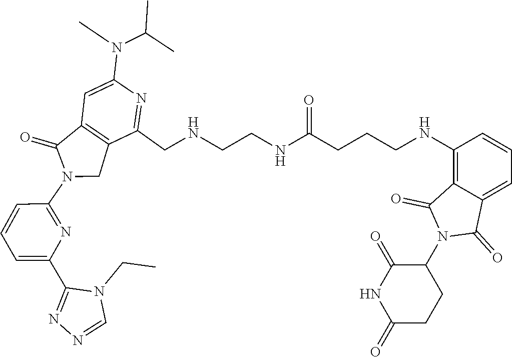 [SMILES: CCn1cnnc1-c1cccc(N2Cc3c(cc(N(C)C(C)C)nc3CNCCNC(=O)CCCNc3cccc4c3C(=O)N(C3CCC(=O)NC3=O)C4=O)C2=O)n1] | 4-((2-(2,6-dioxopiperidin-3-yl)-1,3- dioxoisoindolin-4-yl)amino)-N-(2-(((2- (6-(4-ethyl-4H-1,2,4-triazol-3-yl)pyridin- 2-yl)-6-(isopropyl(methyl)amino)-1- oxo-2,3-dihydro-1H-pyrrolo[3,4- c]pyridin-4- yl)methyl)amino)ethyl)butanamide |
| 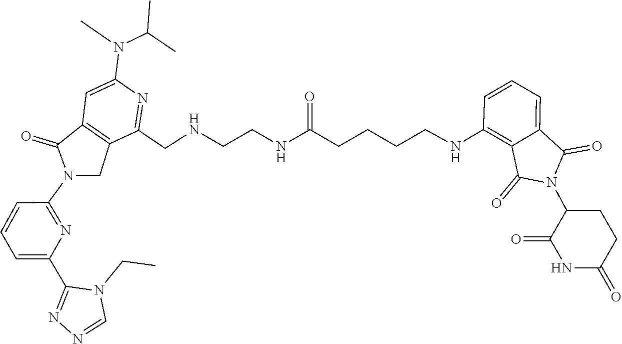 [SMILES: CCn1cnnc1-c1cccc(N2Cc3c(cc(N(C)C(C)C)nc3CNCCNC(=O)CCCCNc3cccc4c3C(=O)N(C3CCC(=O)NC3=O)C4=O)C2=O)n1] | 5-((2-(2,6-dioxopiperidin-3-yl)-1,3- dioxoisoindolin-4-yl)amino)-N-(2-(((2- (6-(4-ethyl-4H-1,2,4-triazol-3-yl)pyridin- 2-yl)-6-(isopropyl(methyl)amino)-1- oxo-2,3-dihydro-1H-pyrrolo[3,4- c]pyridin-4- yl)methyl)amino)ethyl)pentanamide |
| 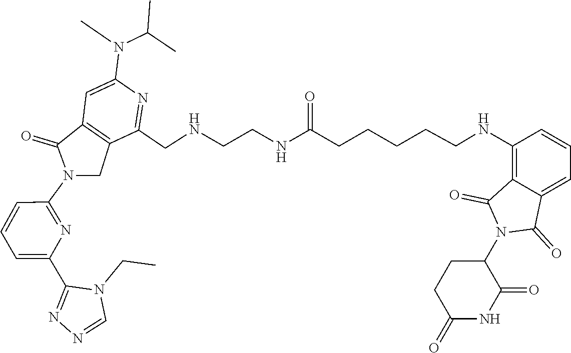 [SMILES: CCn1cnnc1-c1cccc(N2Cc3c(cc(N(C)C(C)C)nc3CNCCNC(=O)CCCCCNc3cccc4c3C(=O)N(C3CCC(=O)NC3=O)C4=O)C2=O)n1] | 6-((2-(2,6-dioxopiperidin-3-yl)-1,3- dioxoisoindolin-4-yl)amino)-N-(2-(((2- (6-(4-ethyl-4H-1,2,4-triazol-3-yl)pyridin- 2-yl)-6-(isopropyl(methyl)amino)-1- oxo-2,3-dihydro-1H-pyrrolo[3,4- c]pyridin-4- yl)methyl)amino)ethyl)hexanamide |
| 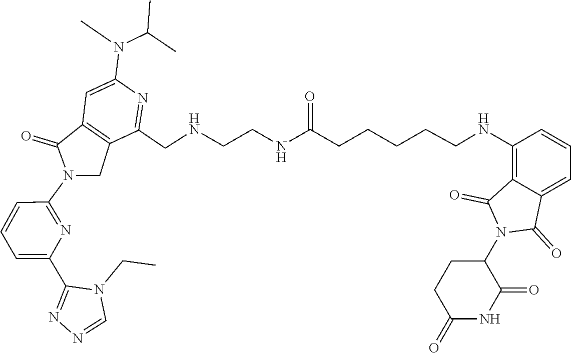 [SMILES: CCn1cnnc1-c1cccc(N2Cc3c(cc(N(C)C(C)C)nc3CNCCNC(=O)CCCCCNc3cccc4c3C(=O)N(C3CCC(=O)NC3=O)C4=O)C2=O)n1] | 7-((2-(2,6-dioxopiperidin-3-yl)-1,3- dioxoisoindolin-4-yl)amino)-N-(2-(((2- (6-(4-ethyl-4H-1,2,4-triazol-3-yl)pyridin- 2-yl)-6-(isopropyl(methyl)amino)-1- oxo-2,3-dihydro-1H-pyrrolo[3,4- c]pyridin-4- yl)methyl)amino)ethyl)heptanamide |
| 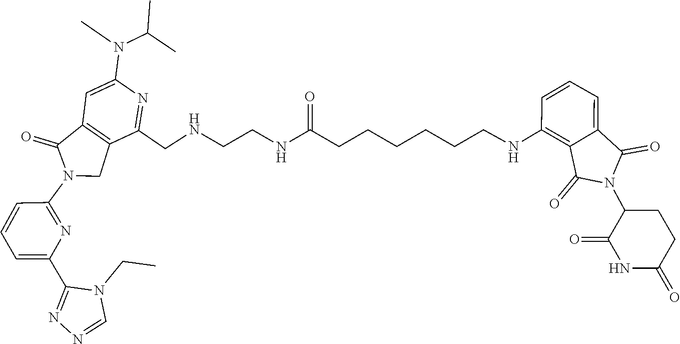 [SMILES: CCn1cnnc1-c1cccc(N2Cc3c(cc(N(C)C(C)C)nc3CNCCNC(=O)CCCCCCNc3cccc4c3C(=O)N(C3CCC(=O)NC3=O)C4=O)C2=O)n1] | 7-((2-(2,6-dioxopiperidin-3-yl)-1,3- dioxoisoindolin-4-yl)amino)-N-(2-(((2- (6-(4-ethyl-4H-1,2,4-triazol-3-yl)pyridin- 2-yl)-6-(isopropyl(methyl)amino)-1- oxo-2,3-dihydro-1H-pyrrolo[3,4- c]pyridin-4- yl)methyl)amino)ethyl)heptanamide |
| 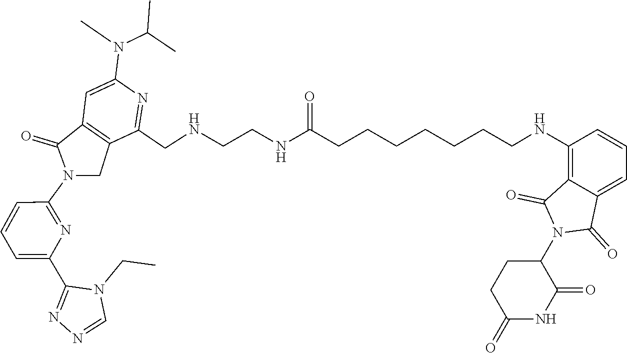 [SMILES: CCn1cnnc1-c1cccc(N2Cc3c(cc(N(C)C(C)C)nc3CNCCNC(=O)CCCCCCCNc3cccc4c3C(=O)N(C3CCC(=O)NC3=O)C4=O)C2=O)n1] | 8-((2-(2,6-dioxopiperidin-3-yl)-1,3- dioxoisoindolin-4-yl)amino)-N-(2-(((2- (6-(4-ethyl-4H-1,2,4-triazol-3-yl)pyridin- 2-yl)-6-(isopropyl(methyl)amino)-1- oxo-2,3-dihydro-1H-pyrrolo[3,4- c]pyridin-4- yl)methyl)amino)ethyl)octanamide |
| 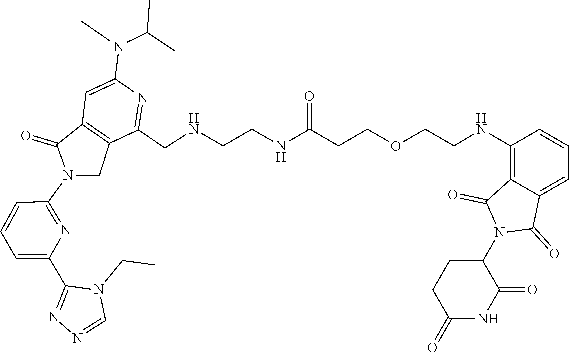 [SMILES: CCn1cnnc1-c1cccc(N2Cc3c(cc(N(C)C(C)C)nc3CNCCNC(=O)CCOCCNc3cccc4c3C(=O)N(C3CCC(=O)NC3=O)C4=O)C2=O)n1] | 3-(2-((2-(2,6-dioxopiperidin-3-yl)-1,3- dioxoisoindolin-4-yl)amino)ethoxy)-N- (2-(((2-(6-(4-ethyl-4H-1,2,4-triazol-3- yl)pyridin-2-yl)-6- (isopropyl(methyl)amino)-1-oxo-2,3- dihydro-1H-pyrrolo[3,4-c]pyridin-4- yl)methyl)amino)ethyl)propanamide |
| 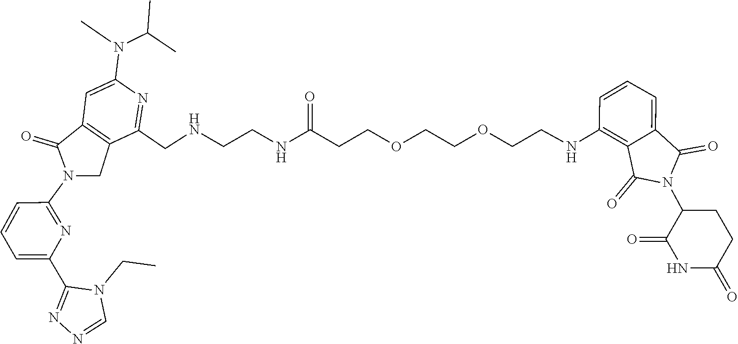 [SMILES: CCn1cnnc1-c1cccc(N2Cc3c(cc(N(C)C(C)C)nc3CNCCNC(=O)CCOCCOCCNc3cccc4c3C(=O)N(C3CCC(=O)NC3=O)C4=O)C2=O)n1] | 3-(2-(2-((2-(2,6-dioxopiperidin-3-yl)- 1,3-dioxoisoindolin-4- yl)amino)ethoxy)ethoxy)-N-(2-(((2-(6- (4-ethyl-4H-1,2,4-triazol-3-yl)pyridin-2- yl)-6-(isopropyl(methyl)amino)-1-oxo- 2,3-dihydro-1H-pyrrolo[3,4-c]pyridin-4- yl)methyl)amino)ethyl)propanamide |
| 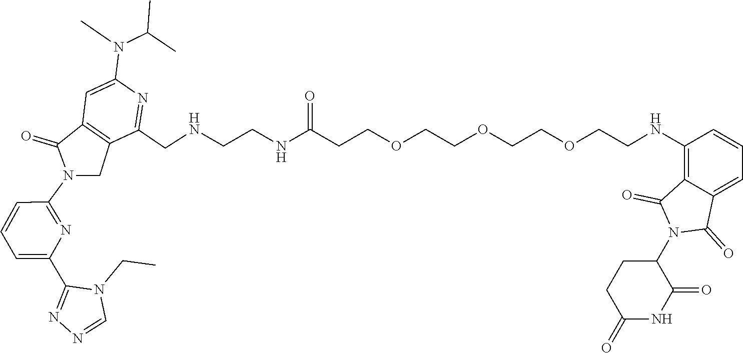 [SMILES: CCn1cnnc1-c1cccc(N2Cc3c(cc(N(C)C(C)C)nc3CNCCNC(=O)CCOCCOCCOCCNc3cccc4c3C(=O)N(C3CCC(=O)NC3=O)C4=O)C2=O)n1] | 3-(2-(2-(2-((2-(2,6-dioxopiperidin-3-yl)- 1,3-dioxoisoindolin-4- yl)amino)ethoxy)ethoxy)ethoxy)-N-(2- (((2-(6-(4-ethyl-4H-1,2,4-triazol-3- yl)pyridin-2-yl)-6- (isopropyl(methyl)amino)-1-oxo-2,3- dihydro-1H-pyrrolo[3,4-c]pyridin-4- yl)methyl)amino)ethyl)propanamide |
| 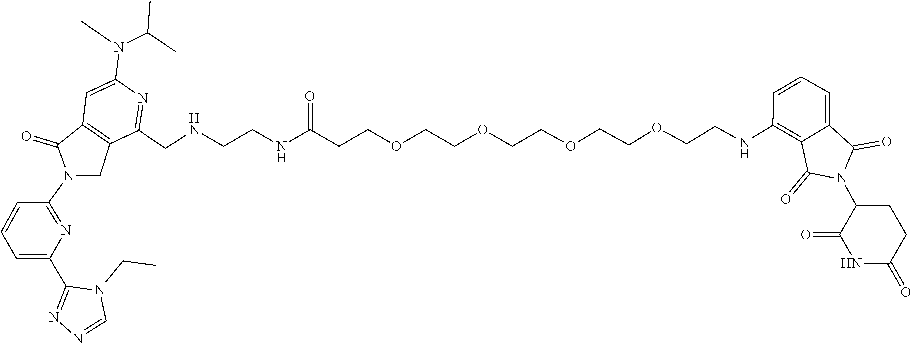 [SMILES: CCn1cnnc1-c1cccc(N2Cc3c(cc(N(C)C(C)C)nc3CNCCNC(=O)CCOCCOCCOCCOCCNc3cccc4c3C(=O)N(C3CCC(=O)NC3=O)C4=O)C2=O)n1] | 1-((2-(2,6-dioxopiperidin-3-yl)-1,3- dioxoisoindolin-4-yl)amino)-N-(2-(((2- (6-(4-ethyl-4H-1,2,4-triazol-3-yl)pyridin- 2-yl)-6-(isopropyl(methyl)amino)-1- oxo-2,3-dihydro-1H-pyrrolo[3,4- c]pyridin-4-yl)methyl)amino)ethyl)- 3,6,9,12-tetraoxapentadecan-15-amide |
| 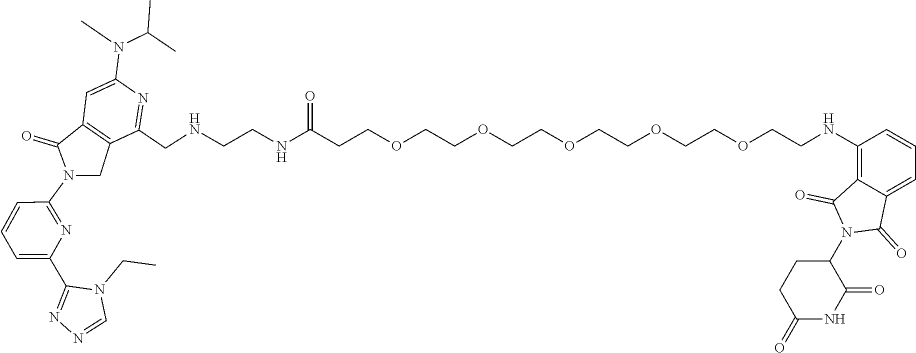 [SMILES: CCn1cnnc1-c1cccc(N2Cc3c(cc(N(C)C(C)C)nc3CNCCNC(=O)CCOCCOCCOCCOCCOCCNc3cccc4c3C(=O)N(C3CCC(=O)NC3=O)C4=O)C2=O)n1] | 1-((2-(2,6-dioxopiperidin-3-yl)-1,3- dioxoisoindolin-4-yl)amino)-N-(2-(((2- (6-(4-ethyl-4H-1,2,4-triazol-3-yl)pyridin- 2-yl)-6-(isopropyl(methyl)amino)-1- oxo-2,3-dihydro-1H-pyrrolo[3,4- c]pyridin-4-yl)methyl)amino)ethyl)- 3,6,9,12-pentaoxaoctadecan-18- amide |
| 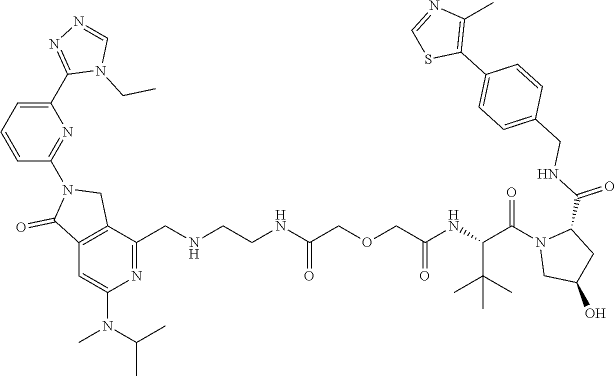 [SMILES: CCn1cnnc1-c1cccc(N2Cc3c(cc(N(C)C(C)C)nc3CNCCNC(=O)COCC(=O)N[C@H](C(=O)N3C[C@H](O)C[C@H]3C(=O)NCc3ccc(-c4scnc4C)cc3)C(C)(C)C)C2=O)n1] | (2S,4R)-1-((S)-12-(tert-butyl)-1-(2-(6- (4-ethyl-4H-1,2,4-triazol-3-yl)pyridin-2- yl)-6-(isopropyl(methyl)amino)-1-oxo- 2,3-dihydro-1H-pyrrolo[3,4-c]pyridin-4- yl)-6,10-dioxo-8-oxa-2,5,11- triazatridecan-13-oyl)-4-hydroxy-N-(4- (4-methylthiazol-5- yl)benzyl)pyrrolidine-2-carboxamide |
| 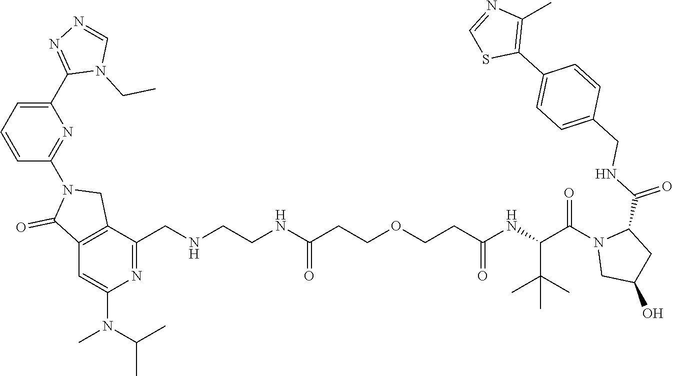 [SMILES: CCn1cnnc1-c1cccc(N2Cc3c(cc(N(C)C(C)C)nc3CNCCNC(=O)CCOCCC(=O)N[C@H](C(=O)N3C[C@H](O)C[C@H]3C(=O)NCc3ccc(-c4scnc4C)cc3)C(C)(C)C)C2=O)n1] | (2S,4R)-1-((S)-14-(tert-butyl)-1-(2-(6- (4-ethyl-4H-1,2,4-triazol-3-yl)pyridin-2- yl)-6-(isopropyl(methyl)amino)-1-oxo- 2,3-dihydro-1H-pyrrolo[3,4-c]pyridin-4- yl)-6,12-dioxo-9-oxa-2,5,13- triazapentadecan-15-oyl)-4-hydroxy-N- (4-(4-methylthiazol-5- yl)benzyl)pyrrolidine-2-carboxamide |
| 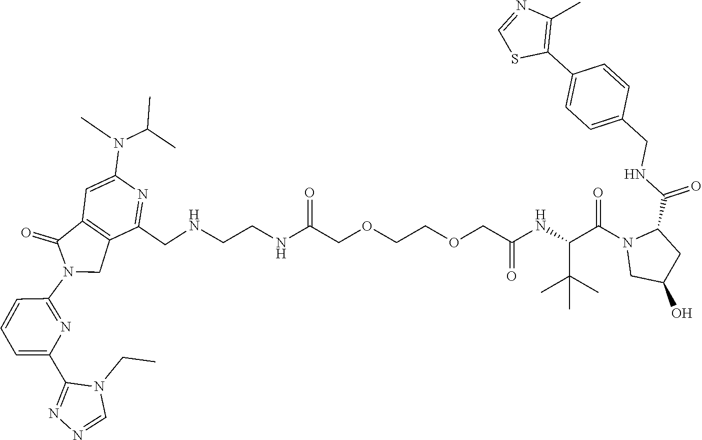 [SMILES: CCn1cnnc1-c1cccc(N2Cc3c(cc(N(C)C(C)C)nc3CNCCNC(=O)COCCOCC(=O)N[C@H](C(=O)N3C[C@H](O)C[C@H]3C(=O)NCc3ccc(-c4scnc4C)cc3)C(C)(C)C)C2=O)n1] | (2S,4R)-1-((S)-15-(tert-butyl)-1-(2-(6- (4-ethyl-4H-1,2,4-triazol-3-yl)pyridin-2- yl)-6-(isopropyl(methyl)amino)-1-oxo- 2,3-dihydro-1H-pyrrolo[3,4-c]pyridin-4- yl)-6,13-dioxo-8,11-dioxa-2,5,14- triazahexadecan-16-oyl)-4-hydroxy-N- (4-(4-methylthiazol-5- yl)benzyl)pyrrolidine-2-carboxamide |
| 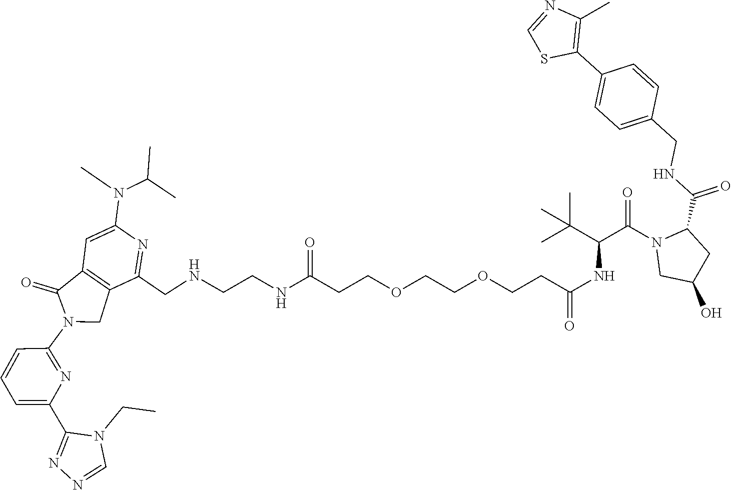 [SMILES: CCn1cnnc1-c1cccc(N2Cc3c(cc(N(C)C(C)C)nc3CNCCNC(=O)CCOCCOCCC(=O)N[C@H](C(=O)N3C[C@H](O)C[C@H]3C(=O)NCc3ccc(-c4scnc4C)cc3)C(C)(C)C)C2=O)n1] | (2S,4R)-1-((S)-17-(tert-butyl)-1-(2-(6- (4-ethyl-4H-1,2,4-triazol-3-yl)pyridin-2- yl)-6-(isopropyl(methyl)amino)-1-oxo- 2,3-dihydro-1H-pyrrolo[3,4-c]pyridin-4- yl)-6,15-dioxo-9,12-dioxa-2,5,16- triazaoctadecan-18-oyl)-4-hydroxy-N- (4-(4-methylthiazol-5- yl)benzyl)pyrrolidine-2-carboxamide |
| 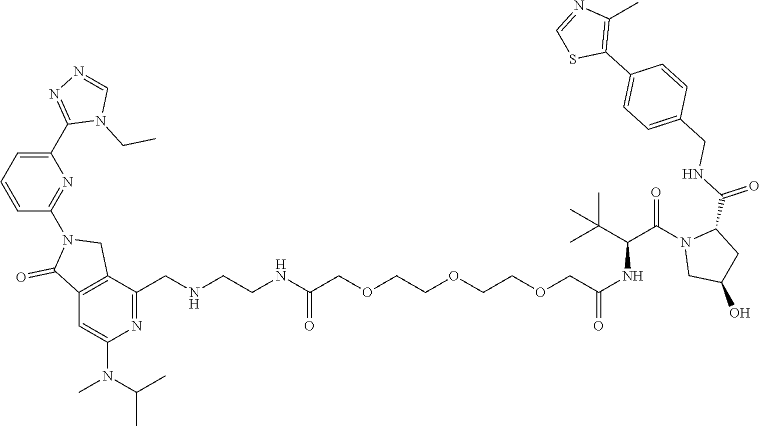 [SMILES: CCn1cnnc1-c1cccc(N2Cc3c(cc(N(C)C(C)C)nc3CNCCNC(=O)COCCOCCOCC(=O)N[C@H](C(=O)N3C[C@H](O)C[C@H]3C(=O)NCc3ccc(-c4scnc4C)cc3)C(C)(C)C)C2=O)n1] | (2S,4R)-1-((S)-18-(tert-butyl)-1-(2-(6- (4-ethyl-4H-1,2,4-triazol-3-yl)pyridin-2- yl)-6-(isopropyl(methyl)amino)-1-oxo- 2,3-dihydro-1H-pyrrolo[3,4-c]pyridin-4- yl)-6,16-dioxo-8,11,14-trioxa-2,5,17- triazanonadecan-19-oyl)-4-hydroxy-N- (4-(4-methylthiazol-5- yl)benzyl)pyrrolidine-2-carboxamide |
| 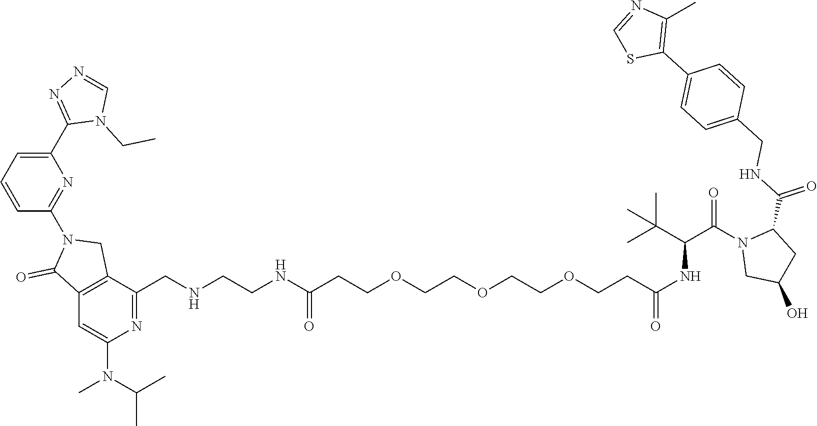 [SMILES: CCn1cnnc1-c1cccc(N2Cc3c(cc(N(C)C(C)C)nc3CNCCNC(=O)CCOCCOCCOCCC(=O)N[C@H](C(=O)N3C[C@H](O)C[C@H]3C(=O)NCc3ccc(-c4scnc4C)cc3)C(C)(C)C)C2=O)n1] | (2S,4R)-1-((S)-20-(tert-butyl)-1-(2-(6- (4-ethyl-4H-1,2,4-triazol-3-yl)pyridin-2- yl)-6-(isopropyl(methyl)amino)-1-oxo- 2,3-dihydro-1H-pyrrolo[3,4-c]pyridin-4- yl)-6,18-dioxo-9,12,15-trioxa-2,5,19- triazahenicosan-21-oyl)-4-hydroxy-N- (4-(4-methylthiazol-5- yl)benzyl)pyrrolidine-2-carboxamide |
| 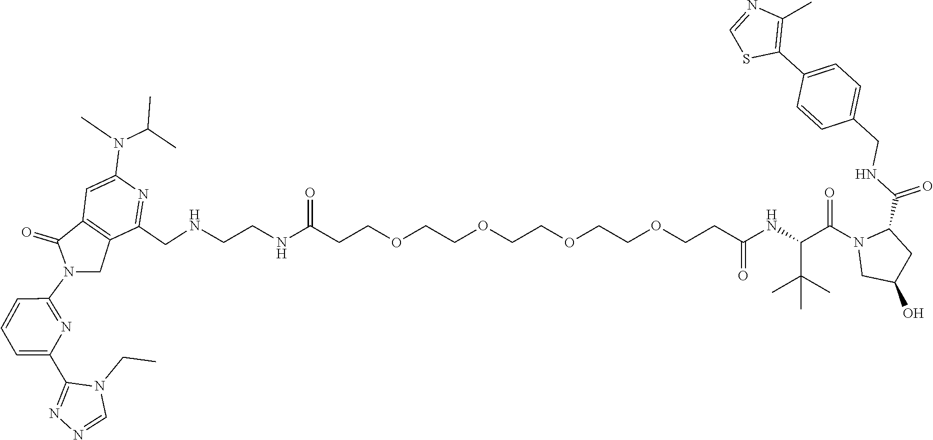 [SMILES: CCn1cnnc1-c1cccc(N2Cc3c(cc(N(C)C(C)C)nc3CNCCNC(=O)CCOCCOCCOCCOCCC(=O)N[C@H](C(=O)N3C[C@H](O)C[C@H]3C(=O)NCc3ccc(-c4scnc4C)cc3)C(C)(C)C)C2=O)n1] | N¹-(2-(((2-(6-(4-ethyl-4H-1,2,4-triazol- 3-yl)pyridin-2-yl)-6- (isopropyl(methyl)amino)-1-oxo-2,3- dihydro-1H-pyrrolo[3,4-c]pyridin-4- yl)methyl)amino)ethyl)-N¹⁶-((S)-1- ((2S,4R)-4-hydroxy-2-((4-(4- methylthiazol-5- yl)benzyl)carbamoyl)pyrrolidin-1-yl)- 3,3-dimethyl-1-oxobutan-2-yl)- 4,7,10,13-tetraoxahexadecanediamide |
| 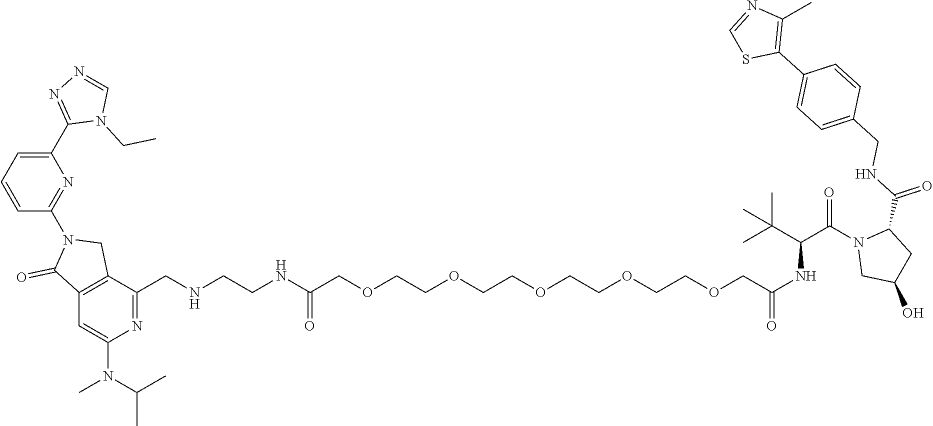 [SMILES: CCn1cnnc1-c1cccc(N2Cc3c(cc(N(C)C(C)C)nc3CNCCNC(=O)COCCOCCOCCOCCOCC(=O)N[C@H](C(=O)N3C[C@H](O)C[C@H]3C(=O)NCc3ccc(-c4scnc4C)cc3)C(C)(C)C)C2=O)n1] | N¹-(2-(((2-(6-(4-ethyl-4H-1,2,4-triazol- 3-yl)pyridin-2-yl)-6- (isopropyl(methyl)amino)-1-oxo-2,3- dihydro-1H-pyrrolo[3,4-c]pyridin-4- yl)methyl)amino)ethyl)-N¹⁷-((S)-1- ((2S,4R)-4-hydroxy-2-((4-(4- methylthiazol-5- yl)benzyl)carbamoyl)pyrrolidin-1-yl)- 3,3-dimethyl-1-oxobutan-2-yl)- 3,6,9,12,15- pentaoxaheptadecanediamide |
| 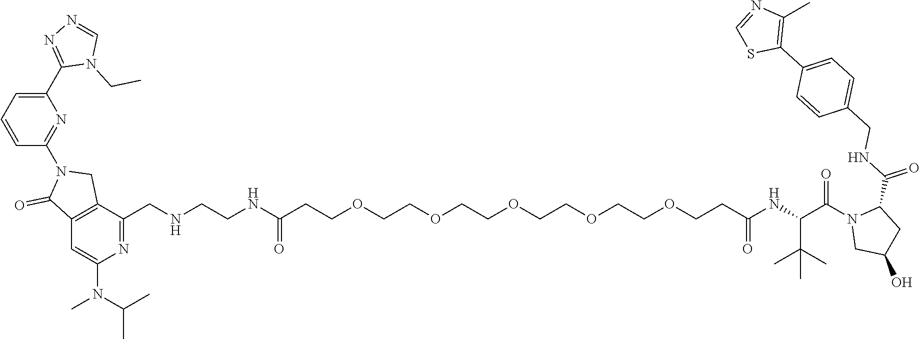 [SMILES: CCn1cnnc1-c1cccc(N2Cc3c(cc(N(C)C(C)C)nc3CNCCNC(=O)CCOCCOCCOCCOCCOCCC(=O)N[C@H](C(=O)N3C[C@H](O)C[C@H]3C(=O)NCc3ccc(-c4scnc4C)cc3)C(C)(C)C)C2=O)n1] | N¹-(2-(((2-(6-(4-ethyl-4H-1,2,4-triazol- 3-yl)pyridin-2-yl)-6- (isopropyl(methyl)amino)-1-oxo-2,3- dihydro-1H-pyrrolo[3,4-c]pyridin-4- yl)methyl)amino)ethyl)-N¹⁹-((S)-1- ((2S,4R)-4-hydroxy-2-((4-(4- methylthiazol-5- yl)benzyl)carbamoyl)pyrrolidin-1-yl)- 3,3-dimethyl-1-oxobutan-2-yl)- 4,7,10,13,16- pentaoxanonadecanediamide |
| 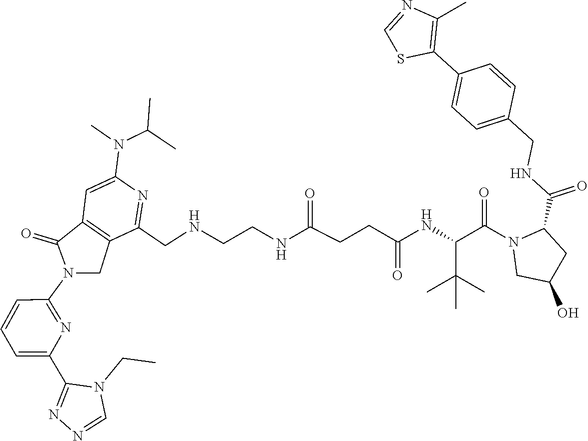 [SMILES: CCn1cnnc1-c1cccc(N2Cc3c(cc(N(C)C(C)C)nc3CNCCNC(=O)CCC(=O)N[C@H](C(=O)N3C[C@H](O)C[C@H]3C(=O)NCc3ccc(-c4scnc4C)cc3)C(C)(C)C)C2=O)n1] | N¹-(2-(((2-(6-(4-ethyl-4H-1,2,4-triazol- 3-yl)pyridin-2-yl)-6- (isopropyl(methyl)amino)-1-oxo-2,3- dihydro-1H-pyrrolo[3,4-c]pyridin-4- yl)methyl)amino)ethyl)-N⁴-((S)-1- ((2S,4R)-4-hydroxy-2-((4-(4- methylthiazol-5- yl)benzyl)carbamoyl)pyrrolidin-1-yl)- 3,3-dimethyl-1-oxobutan-2- yl)succinamide |
| 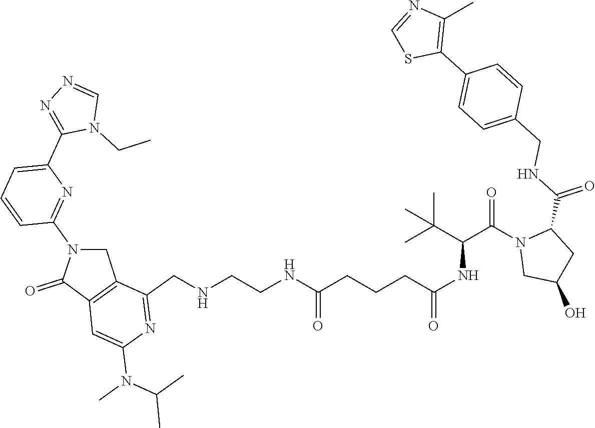 [SMILES: CCn1cnnc1-c1cccc(N2Cc3c(cc(N(C)C(C)C)nc3CNCCNC(=O)CCCC(=O)N[C@H](C(=O)N3C[C@H](O)C[C@H]3C(=O)NCc3ccc(-c4scnc4C)cc3)C(C)(C)C)C2=O)n1] | N¹-(2-(((2-(6-(4-ethyl-4H-1,2,4-triazol- 3-yl)pyridin-2-yl)-6- (isopropyl(methyl)amino)-1-oxo-2,3- dihydro-1H-pyrrolo[3,4-c]pyridin-4- yl)methyl)amino)ethyl)-N⁵-((S)-1- ((2S,4R)-4-hydroxy-2-((4-(4- methylthiazol-5- yl)benzyl)carbamoyl)pyrrolidin-1-yl)- 3,3-dimethyl-1-oxobutan-2- yl)glutaramide |
| 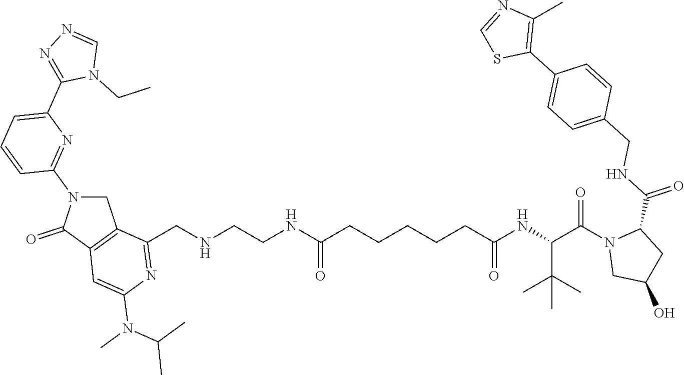 [SMILES: CCn1cnnc1-c1cccc(N2Cc3c(cc(N(C)C(C)C)nc3CNCCNC(=O)CCCCCC(=O)N[C@H](C(=O)N3C[C@H](O)C[C@H]3C(=O)NCc3ccc(-c4scnc4C)cc3)C(C)(C)C)C2=O)n1] | N¹-(2-(((2-(6-(4-ethyl-4H-1,2,4-triazol- 3-yl)pyridin-2-yl)-6- (isopropyl(methyl)amino)-1-oxo-2,3- dihydro-1H-pyrrolo[3,4-c]pyridin-4- yl)methyl)amino)ethyl)-N⁷-((S)-1- ((2S,4R)-4-hydroxy-2-((4-(4- methylthiazol-5- yl)benzyl)carbamoyl)pyrrolidin-1-yl)- 3,3-dimethyl-1-oxobutan-2- yl)heptanediamide |
| 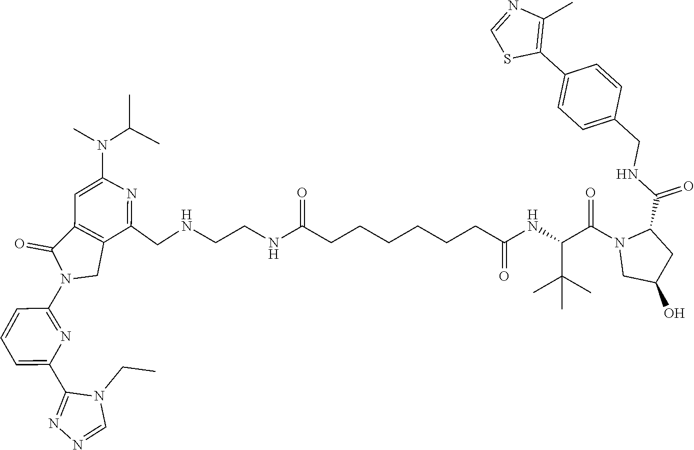 [SMILES: CCn1cnnc1-c1cccc(N2Cc3c(cc(N(C)C(C)C)nc3CNCCNC(=O)CCCCCCC(=O)N[C@H](C(=O)N3C[C@H](O)C[C@H]3C(=O)NCc3ccc(-c4scnc4C)cc3)C(C)(C)C)C2=O)n1] | N¹-(2-(((2-(6-(4-ethyl-4H-1,2,4-triazol- 3-yl)pyridin-2-yl)-6- (isopropyl(methyl)amino)-1-oxo-2,3- dihydro-1H-pyrrolo[3,4-c]pyridin-4- yl)methyl)amino)ethyl)-N⁸-((S)-1- ((2S,4R)-4-hydroxy-2-((4-(4- methylthiazol-5- yl)benzyl)carbamoyl)pyrrolidin-1-yl)- 3,3-dimethyl-1-oxobutan-2- yl)octanediamide |
| 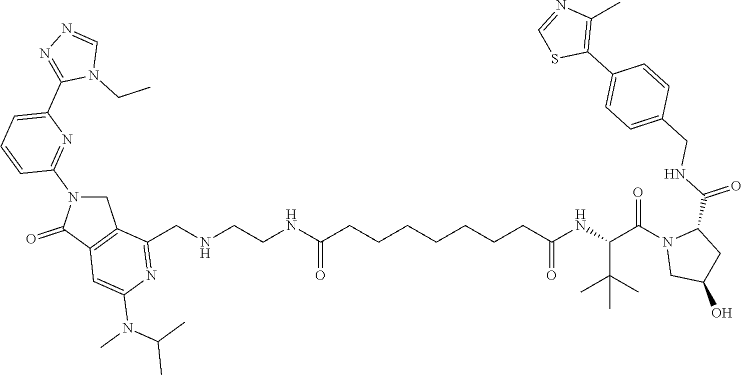 [SMILES: CCn1cnnc1-c1cccc(N2Cc3c(cc(N(C)C(C)C)nc3CNCCNC(=O)CCCCCCCC(=O)N[C@H](C(=O)N3C[C@H](O)C[C@H]3C(=O)NCc3ccc(-c4scnc4C)cc3)C(C)(C)C)C2=O)n1] | N¹-(2-(((2-(6-(4-ethyl-4H-1,2,4-triazol- 3-yl)pyridin-2-yl)-6- (isopropyl(methyl)amino)-1-oxo-2,3- dihydro-1H-pyrrolo[3,4-c]pyridin-4- yl)methyl)amino)ethyl)-N⁹-((S)-1- ((2S,4R)-4-hydroxy-2-((4-(4- methylthiazol-5- yl)benzyl)carbamoyl)pyrrolidin-1-yl)- 3,3-dimethyl-1-oxobutan-2- yl)nonanediamide |
| 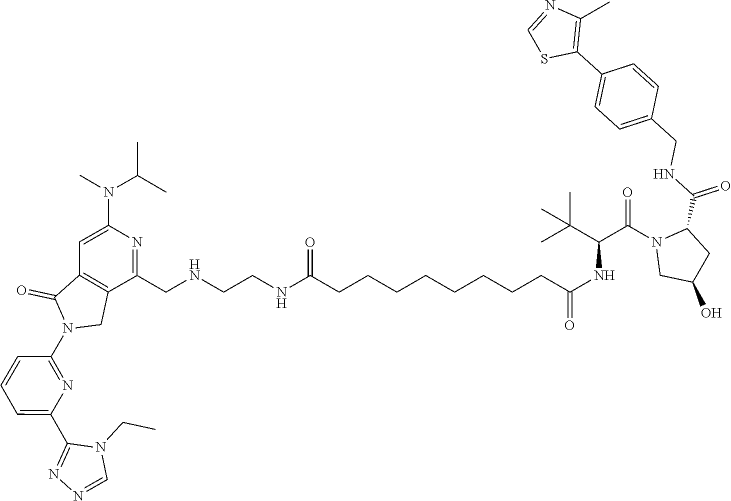 [SMILES: CCn1cnnc1-c1cccc(N2Cc3c(cc(N(C)C(C)C)nc3CNCCNC(=O)CCCCCCCCC(=O)N[C@H](C(=O)N3C[C@H](O)C[C@H]3C(=O)NCc3ccc(-c4scnc4C)cc3)C(C)(C)C)C2=O)n1] | N¹-(2-(((2-(6-(4-ethyl-4H-1,2,4-triazol- 3-yl)pyridin-2-yl)-6- (isopropyl(methyl)amino)-1-oxo-2,3- dihydro-1H-pyrrolo[2,3-c]pyridin-4- yl)methyl)amino)ethyl)-N¹⁰-((S)-1- ((2S,4R)-4-hydroxy-2-((4-(4- methylthiazol-5- yl)benzyl)carbamoyl)pyrrolidin-1-yl)- 3,3-dimethyl-1-oxobutan-2- yl)decanediamide |
| 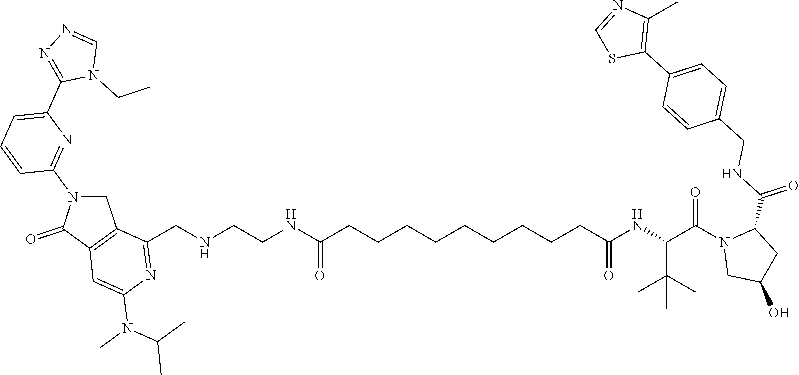 [SMILES: CCn1cnnc1-c1cccc(N2Cc3c(cc(N(C)C(C)C)nc3CNCCNC(=O)CCCCCCCCCC(=O)N[C@H](C(=O)N3C[C@H](O)C[C@H]3C(=O)NCc3ccc(-c4scnc4C)cc3)C(C)(C)C)C2=O)n1] | N¹-(2-(((2-(6-(4-ethyl-4H-1,2,4-triazol- 3-yl)pyridin-2-yl)-6- (isopropyl(methyl)amino)-1-oxo-2,3- dihydro-1H-pyrrolo[3,4-c]pyridin-4- yl)methyl)amino)ethyl)-N¹¹-((S)-1- ((2S,4R)-4-hydroxy-2-((4-(4- methylthiazol-5- yl)benzyl)carbamoyl)pyrrolidin-1-yl)- 3,3-dimethyl-1-oxobutan-2- yl)undecanediamide |
| 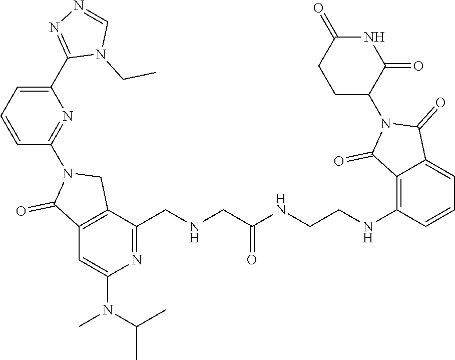 [SMILES: CCn1cnnc1-c1cccc(N2Cc3c(cc(N(C)C(C)C)nc3CNCC(=O)NCCNc3cccc4c3C(=O)N(C3CCC(=O)NC3=O)C4=O)C2=O)n1] | N-(2-((2-(2,6-dioxopiperidin-3-yl)-1,3- dioxoisoindolin-4-yl)amino)ethyl)-2- (((2-(6-(4-ethyl-4H-1,2,4-triazol-3- yl)pyridin-2-yl)-6- (isopropyl(methyl)amino)-1-oxo-2,3- dihydro-1H-pyrrolo[3,4-c]pyridin-4- yl)methyl)amino)acetamide |
| 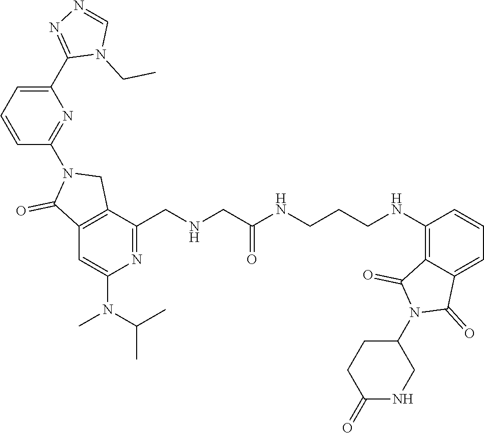 [SMILES: CCn1cnnc1-c1cccc(N2Cc3c(cc(N(C)C(C)C)nc3CNCC(=O)NCCCNc3cccc4c3C(=O)N(C3CCC(=O)NC3)C4=O)C2=O)n1] | N-(3-((2-(2,6-dioxopiperidin-3-yl)-1,3- dioxoisoindolin-4-yl)amino)propyl)-2- (((2-(6-(4-ethyl-4H-1,2,4-triazol-3- yl)pyridin-2-yl)-6- (isopropyl(methyl)amino)-1-oxo-2,3- dihydro-1H-pyrrolo[3,4-c]pyridin-4- yl)methyl)amino)acetamide |
| 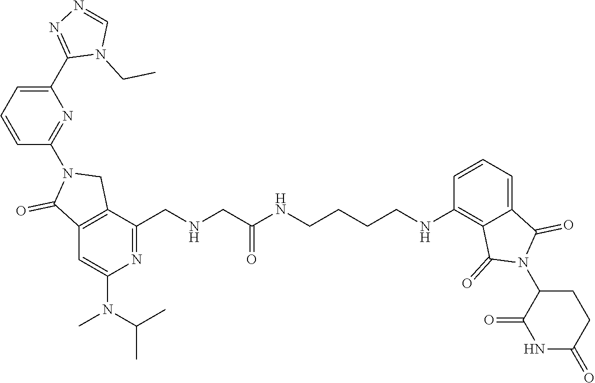 [SMILES: CCn1cnnc1-c1cccc(N2Cc3c(cc(N(C)C(C)C)nc3CNCC(=O)NCCCCNc3cccc4c3C(=O)N(C3CCC(=O)NC3=O)C4=O)C2=O)n1] | N-(4-((2-(2,6-dioxopiperidin-3-yl)-1,3- dioxoisoindolin-4-yl)amino)butyl)-2- (((2-(6-(4-ethyl-4H-1,2,4-triazol-3- yl)pyridin-2-yl)-6- (isopropyl(methyl)amino)-1-oxo-2,3- dihydro-1H-pyrrolo[3,4-c]pyridin-4- yl)methyl)amino)acetamide |
| 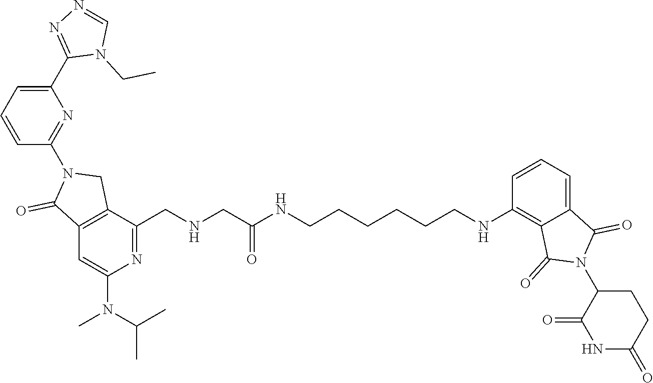 [SMILES: CCn1cnnc1-c1cccc(N2Cc3c(cc(N(C)C(C)C)nc3CNCC(=O)NCCCCCCNc3cccc4c3C(=O)N(C3CCC(=O)NC3=O)C4=O)C2=O)n1] | N-(5-((2-(2,6-dioxopiperidin-3-yl)-1,3- dioxoisoindolin-4-yl)amino)pentyl)-2- (((2-(6-(4-ethyl-4H-1,2,4-triazol-3- yl)pyridin-2-yl)-6- (isopropyl(methyl)amino)-1-oxo-2,3- dihydro-1H-pyrrolo[3,4-c]pyridin-4- yl)methyl)amino)acetamide |
| 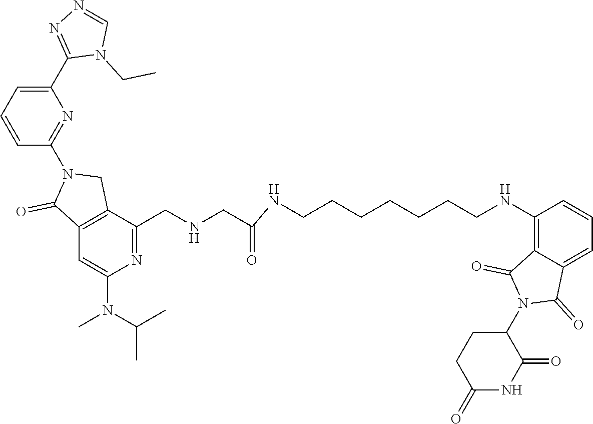 [SMILES: CCn1cnnc1-c1cccc(N2Cc3c(cc(N(C)C(C)C)nc3CNCC(=O)NCCCCCCCNc3cccc4c3C(=O)N(C3CCC(=O)NC3=O)C4=O)C2=O)n1] | N-(7-((2-(2,6-dioxopiperidin-3-yl)-1,3- dioxoisoindolin-4-yl)amino)heptyl)-2- (((2-(6-(4-ethyl-4H-1,2,4-triazol-3- yl)pyridin-2-yl)-6- (isopropyl(methyl)amino)-1-oxo-2,3- dihydro-1H-pyrrolo[3,4-c]pyridin-4- yl)methyl)amino)acetamide |
| 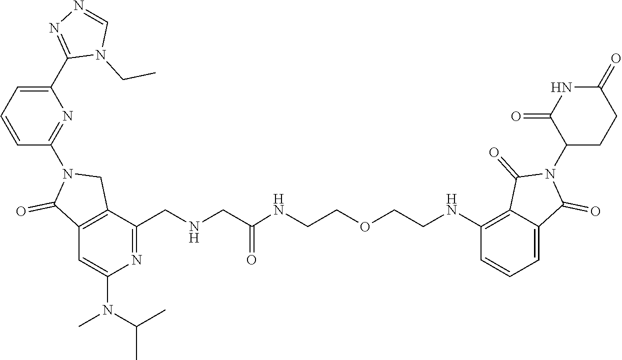 [SMILES: CCn1cnnc1-c1cccc(N2Cc3c(cc(N(C)C(C)C)nc3CNCC(=O)NCCOCCNc3cccc4c3C(=O)N(C3CCC(=O)NC3=O)C4=O)C2=O)n1] | N-(8-((2-(2,6-dioxopiperidin-3-yl)-1,3- dioxoisoindolin-4-yl)amino)octyl)-2- (((2-(6-(4-ethyl-4H-1,2,4-triazol-3- yl)pyridin-2-yl)-6- (isopropyl(methyl)amino)-1-oxo-2,3- dihydro-1H-pyrrolo[3,4-c]pyridin-4- yl)methyl)amino)acetamide |
| 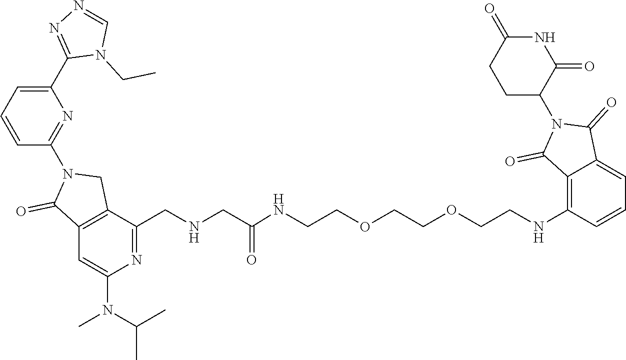 [SMILES: CCn1cnnc1-c1cccc(N2Cc3c(cc(N(C)C(C)C)nc3CNCC(=O)NCCOCCOCCNc3cccc4c3C(=O)N(C3CCC(=O)NC3=O)C4=O)C2=O)n1] | N-(2-(2-(2-((2-(2,6-dioxopiperidin-3-yl)- 1,3-dioxoisoindolin-4- yl)amino)ethoxy)ethoxy)ethyl)-2-(((2- (6-(4-ethyl-4H-1,2,4-triazol-3-yl)pyridin- 2-yl)-6-(isopropyl(methyl)amino)-1- oxo-2,3-dihydro-1H-pyrrolo[3,4- c]pyridin-4-yl)methyl)amino)acetamide |
| 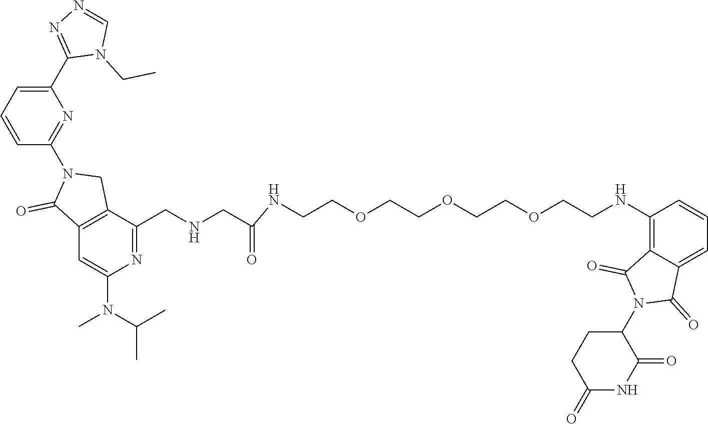 [SMILES: CCn1cnnc1-c1cccc(N2Cc3c(cc(N(C)C(C)C)nc3CNCC(=O)NCCOCCOCCOCCNc3cccc4c3C(=O)N(C3CCC(=O)NC3=O)C4=O)C2=O)n1] | N-(2-(2-(2-(2-((2-(2,6-dioxopiperidin-3- yl)-1,3-dioxoisoindolin-4- yl)amino)ethoxy)ethoxy)ethoxy)ethyl)- 2-(((2-(6-(4-ethyl-4H-1,2,4-triazol-3- yl)pyridin-2-yl)-6- (isopropyl(methyl)amino)-1-oxo-2,3- dihydro-1H-pyrrolo[3,4-c]pyridin-4- yl)methyl)amino)acetamide |
| 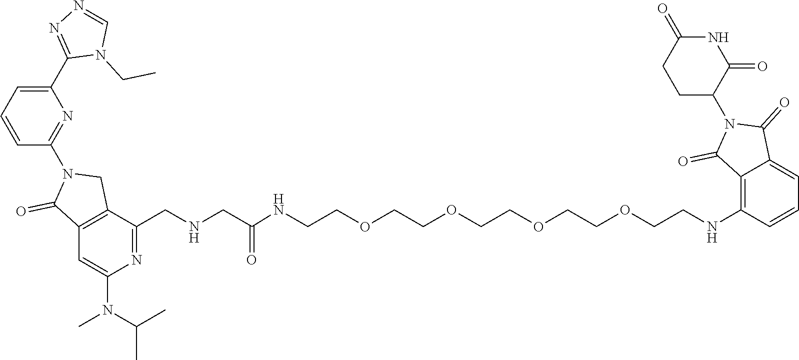 [SMILES: CCn1cnnc1-c1cccc(N2Cc3c(cc(N(C)C(C)C)nc3CNCC(=O)NCCOCCOCCOCCOCCNc3cccc4c3C(=O)N(C3CCC(=O)NC3=O)C4=O)C2=O)n1] | N-(14-((2-(2,6-dioxopiperidin-3-yl)-1,3- dioxoisoindolin-4-yl)amino)-3,6,9,12- tetraoxatetradecyl)-2-(((2-(6-(4-ethyl- 4H-1,2,4-triazol-3-yl)pyridin-2-yl)-6- (isopropyl(methyl)amino)-1-oxo-2,3- dihydro-1H-pyrrolo[3,4-c]pyridin-4- yl)methyl)amino)acetamide |
| 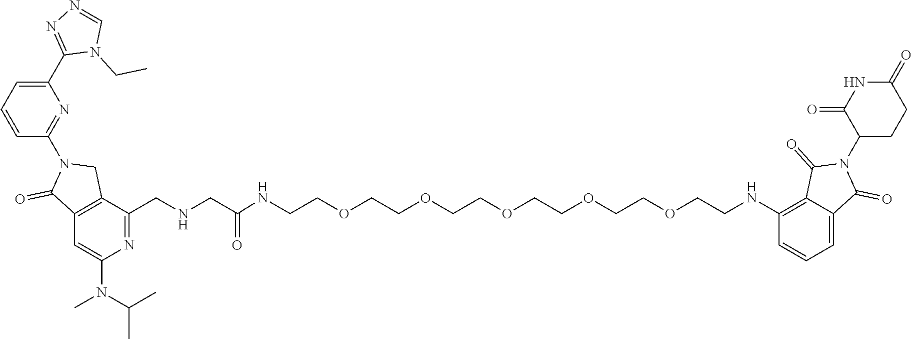 [SMILES: CCn1cnnc1-c1cccc(N2Cc3c(cc(N(C)C(C)C)nc3CNCC(=O)NCCOCCOCCOCCOCCOCCNc3cccc4c3C(=O)N(C3CCC(=O)NC3=O)C4=O)C2=O)n1] | N-(17-((2-(2,6-dioxopiperidin-3-yl)-1,3- dioxoisoindolin-4-yl)amino)- 3,6,9,12,15-pentaoxaheptadecyl)-2- (((2-(6-(4-ethyl-4H-1,2,4-triazol-3- yl)pyridin-2-yl)-6- (isopropyl(methyl)amino)-1-oxo-2,3- dihydro-1H-pyrrolo[3,4-c]pyridin-4- yl)methyl)amino)acetamide |
| 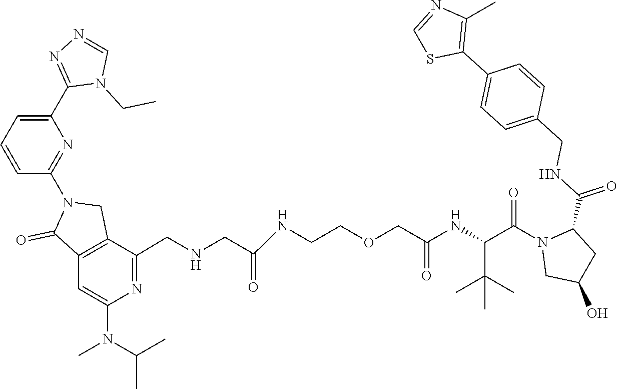 [SMILES: CCn1cnnc1-c1cccc(N2Cc3c(cc(N(C)C(C)C)nc3CNCC(=O)NCCOCC(=O)N[C@H](C(=O)N3C[C@H](O)C[C@H]3C(=O)NCc3ccc(-c4scnc4C)cc3)C(C)(C)C)C2=O)n1] | (2S,4R)-1-((S)-12-(tert-butyl)-1-(2-(6- (4-ethyl-4H-1,2,4-triazol-3-yl)pyridin-2- yl)-6-(isopropyl(methyl)amino)-1-oxo- 2,3-dihydro-1H-pyrrolo[3,4-c]pyridin-4- yl)-4,10-dioxo-8-oxa-2,5,11- triazatridecan-13-oyl)-4-hydroxy-N-(4- (4-methylthiazol-5- yl)benzyl)pyrrolidine-2-carboxamide |
| 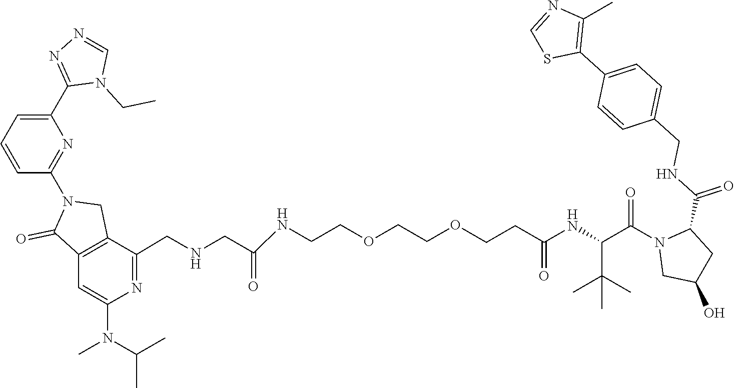 [SMILES: CCn1cnnc1-c1cccc(N2Cc3c(cc(N(C)C(C)C)nc3CNCC(=O)NCCOCCOCCC(=O)N[C@H](C(=O)N3C[C@H](O)C[C@H]3C(=O)NCc3ccc(-c4scnc4C)cc3)C(C)(C)C)C2=O)n1] | (2S,4R)-1-((S)-13-(tert-butyl)-1-(2-(6- (4-ethyl-4H-1,2,4-triazol-3-yl)pyridin-2- yl)-6-(isopropyl(methyl)amino)-1-oxo- 2,3-dihydro-1H-pyrrolo[3,4-c]pyridin-4- yl)-4,11-dioxo-8-oxa-2,5,12- triazatetradecan-14-oyl)-4-hydroxy-N- (4-(4-methylthiazol-5- yl)benzyl)pyrrolidine-2-carboxamide |
| 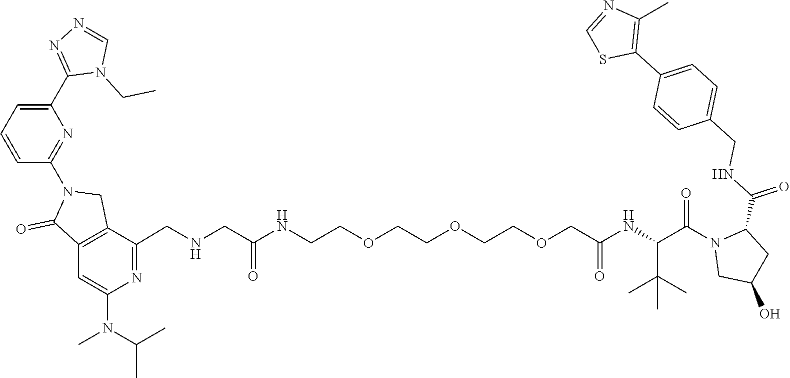 [SMILES: CCn1cnnc1-c1cccc(N2Cc3c(cc(N(C)C(C)C)nc3CNCC(=O)NCCOCCOCCOCC(=O)N[C@H](C(=O)N3C[C@H](O)C[C@H]3C(=O)NCc3ccc(-c4scnc4C)cc3)C(C)(C)C)C2=O)n1] | (2S,4R)-1-((S)-18-(tert-butyl)-1-(2-(6- (4-ethyl-4H-1,2,4-triazol-3-yl)pyridin-2- yl)-6-(isopropyl(methyl)amino)-1-oxo- 2,3-dihydro-1H-pyrrolo[3,4-c]pyridin-4- yl)-4,16-dioxo-8,11,14-trioxa-2,5,17- triazanonadecan-19-oyl)-4-hydroxy-N- (4-(4-methylthiazol-5- yl)benzyl)pyrrolidine-2-carboxamide |
| 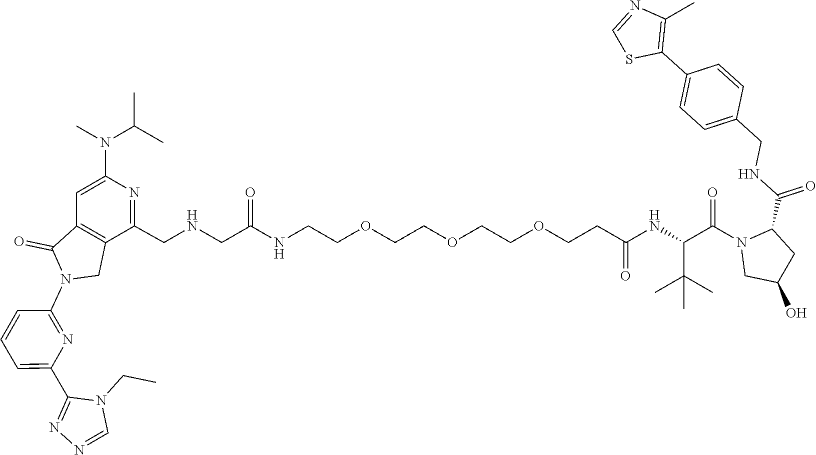 [SMILES: CCn1cnnc1-c1cccc(N2Cc3c(cc(N(C)C(C)C)nc3CNCC(=O)NCCOCCOCCOCCC(=O)N[C@H](C(=O)N3C[C@H](O)C[C@H]3C(=O)NCc3ccc(-c4scnc4C)cc3)C(C)(C)C)C2=O)n1] | (2S,4R)-1-((S)-19-(tert-butyl)-1-(2-(6- (4-ethyl-4H-1,2,4-triazol-3-yl)pyridin-2- yl)-6-(isopropyl(methyl)amino)-1-oxo- 2,3-dihydro-1H-pyrrolo[3,4-c]pyridin-4- yl)-4,17-dioxo-8,11,14-trioxa-2,5,18- triazaicosan-20-oyl)-4-hydroxy-N-(4-(4- methylthiazol-5-yl)benzyl)pyrrolidine-2- carboxamide |
| 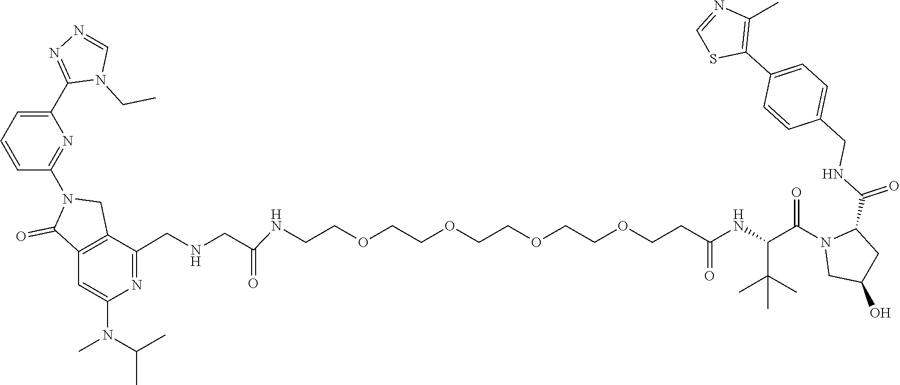 [SMILES: CCn1cnnc1-c1cccc(N2Cc3c(cc(N(C)C(C)C)nc3CNCC(=O)NCCOCCOCCOCCOCCC(=O)N[C@H](C(=O)N3C[C@H](O)C[C@H]3C(=O)NCc3ccc(-c4scnc4C)cc3)C(C)(C)C)C2=O)n1] | (2S,4R)-1-((S)-22-(tert-butyl)-1-(2-(6- (4-ethyl-4H-1,2,4-triazol-3-yl)pyridin-2- yl)-6-(isopropyl(methyl)amino)-1-oxo- 2,3-dihydro-1H-pyrrolo[3,4-c]pyridin-4- yl)-4,20-dioxo-8,11,14,17-tetraoxa- 2,5,21-triazatricosan-23-oyl)-4- hydroxy-N-(4-(4-methylthiazol-5- yl)benzyl)pyrrolidine-2-carboxamide |
| 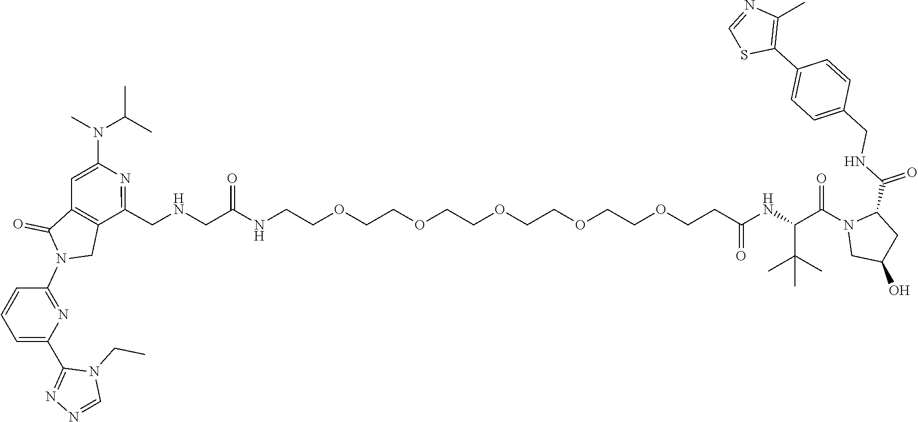 [SMILES: CCn1cnnc1-c1cccc(N2Cc3c(cc(N(C)C(C)C)nc3CNCC(=O)NCCOCCOCCOCCOCCOCCC(=O)N[C@H](C(=O)N3C[C@H](O)C[C@H]3C(=O)NCc3ccc(-c4scnc4C)cc3)C(C)(C)C)C2=O)n1] | (2S,4R)-1-((S)-25-(tert-butyl)-1-(2-(6- (4-ethyl-4H-1,2,4-triazol-3-yl)pyridin-2- yl)-6-(isopropyl(methyl)amino)-1-oxo- 2,3-dihydro-1H-pyrrolo[3,4-c]pyridin-4- yl)-4,23-dioxo-8,11,14,17,20-pentaoxa- 2,5,24-triazahexacosan-26-oyl)-4- hydroxy-N-(4-(4-methylthiazol-5- yl)benzyl)pyrrolidine-2-carboxamide |
| 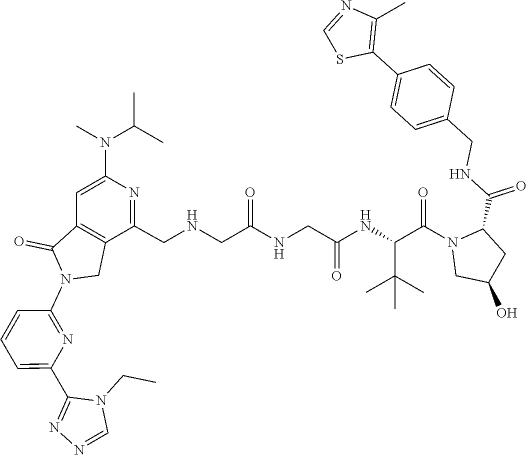 [SMILES: CCn1cnnc1-c1cccc(N2Cc3c(cc(N(C)C(C)C)nc3CNCC(=O)NCC(=O)N[C@H](C(=O)N3C[C@H](O)C[C@H]3C(=O)NCc3ccc(-c4scnc4C)cc3)C(C)(C)C)C2=O)n1] | (2S,4R)-1-((S)-2-(2-(2-(((2-(6-(4-ethyl- 4H-1,2,4-triazol-3-yl)pyridin-2-yl)-6- (isopropyl(methyl)amino)-1-oxo-2,3- dihydro-1H-pyrrolo[3,4-c]pyridin-4- yl)methyl)amino)acetamido)acetamido)- 3,3-dimethylbutanoyl)-4-hydroxy-N-(4- (4-methylthiazol-5- yl)benzyl)pyrrolidine-2-carboxamide |
| 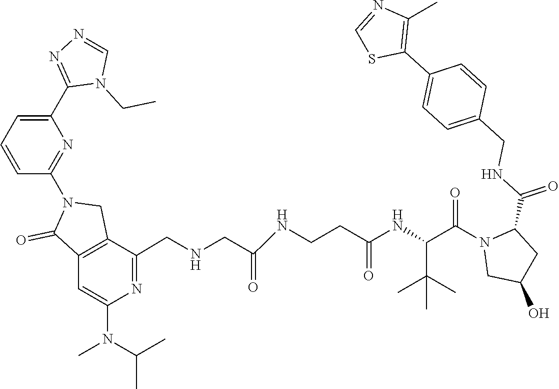 [SMILES: CCn1cnnc1-c1cccc(N2Cc3c(cc(N(C)C(C)C)nc3CNCC(=O)NCCC(=O)N[C@H](C(=O)N3C[C@H](O)C[C@H]3C(=O)NCc3ccc(-c4scnc4C)cc3)C(C)(C)C)C2=O)n1] | (2S,4R)-1-((S)-2-(3-(2-(((2-(6-(4-ethyl- 4H-1,2,4-triazol-3-yl)pyridin-2-yl)-6- (isopropyl(methyl)amino)-1-oxo-2,3- dihydro-1H-pyrrolo[3,4-c]pyridin-4- yl)methyl)amino)acetamido)propanamido)- 3,3-dimethylbutanoyl)-4-hydroxy- N-(4-(4-methylthiazol-5- yl)benzyl)pyrrolidine-2-carboxamide |
| 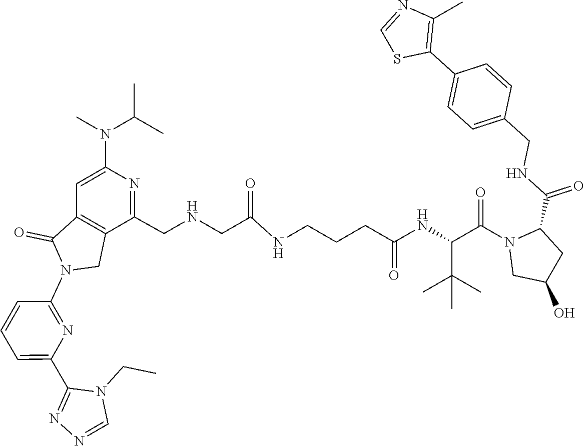 [SMILES: CCn1cnnc1-c1cccc(N2Cc3c(cc(N(C)C(C)C)nc3CNCC(=O)NCCCC(=O)N[C@H](C(=O)N3C[C@H](O)C[C@H]3C(=O)NCc3ccc(-c4scnc4C)cc3)C(C)(C)C)C2=O)n1] | (2S,4R)-1-((S)-2-(4-(2-(((2-(6-(4-ethyl- 4H-1,2,4-triazol-3-yl)pyridin-2-yl)-6- (isopropyl(methyl)amino)-1-oxo-2,3- dihydro-1H-pyrrolo[3,4-c]pyridin-4- yl)methyl)amino)acetamido)butanamido)- 3,3-dimethylbutanoyl)-4-hydroxy-N- (4-(4-methylthiazol-5- yl)benzyl)pyrrolidine-2-carboxamide |
| 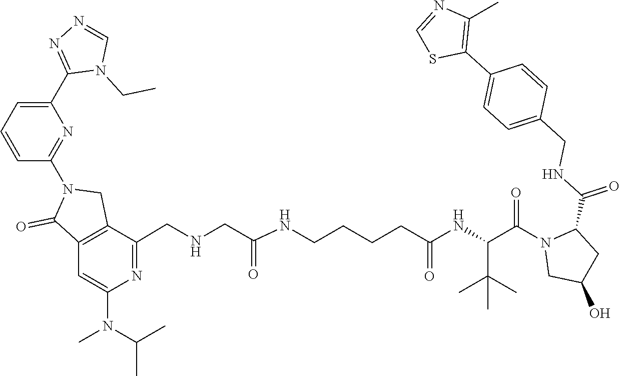 [SMILES: CCn1cnnc1-c1cccc(N2Cc3c(cc(N(C)C(C)C)nc3CNCC(=O)NCCCCC(=O)N[C@H](C(=O)N3C[C@H](O)C[C@H]3C(=O)NCc3ccc(-c4scnc4C)cc3)C(C)(C)C)C2=O)n1] | (2S,4R)-1-((S)-2-(5-(2-(((2-(6-(4-ethyl- 4H-1,2,4-triazol-3-yl)pyridin-2-yl)-6- (isopropyl(methyl)amino)-1-oxo-2,3- dihydro-1H-pyrrolo[3,4-c]pyridin-4- yl)methyl)amino)acetamido)pentanamido)- 3,3-dimethylbutanoyl)-4-hydroxy- N-(4-(4-methylthiazol-5- yl)benzyl)pyrrolidine-2-carboxamide |
| 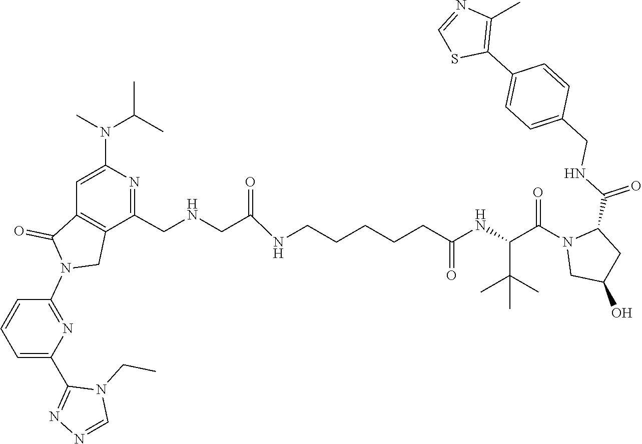 [SMILES: CCn1cnnc1-c1cccc(N2Cc3c(cc(N(C)C(C)C)nc3CNCC(=O)NCCCCCC(=O)N[C@H](C(=O)N3C[C@H](O)C[C@H]3C(=O)NCc3ccc(-c4scnc4C)cc3)C(C)(C)C)C2=O)n1] | (2S,4R)-1-((S)-2-(6-(2-(((2-(6-(4-ethyl- 4H-1,2,4-triazol-3-yl)pyridin-2-yl)-6- (isopropyl(methyl)amino)-1-oxo-2,3- dihydro-1H-pyrrolo[3,4-c]pyridin-4- yl)methyl)amino)acetamido)hexanamido)- 3,3-dimethylbutanoyl)-4-hydroxy-N- (4-(4-methylthiazol-5- yl)benzyl)pyrrolidine-2-carboxamide |
| 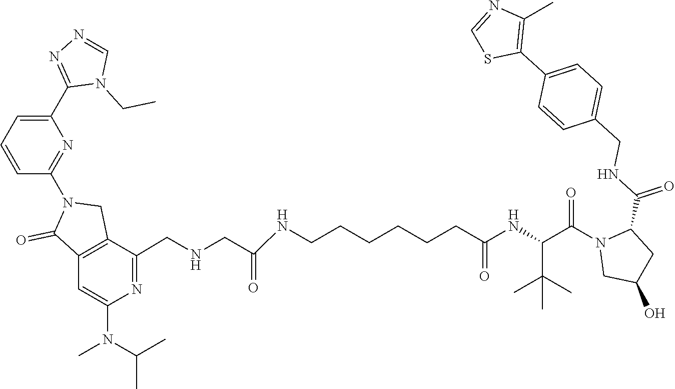 [SMILES: CCn1cnnc1-c1cccc(N2Cc3c(cc(N(C)C(C)C)nc3CNCC(=O)NCCCCCCC(=O)N[C@H](C(=O)N3C[C@H](O)C[C@H]3C(=O)NCc3ccc(-c4scnc4C)cc3)C(C)(C)C)C2=O)n1] | (2S,4R)-1-((S)-2-(7-(2-(((2-(6-(4-ethyl- 4H-1,2,4-triazol-3-yl)pyridin-2-yl)-6- (isopropyl(methyl)amino)-1-oxo-2,3- dihydro-1H-pyrrolo[3,4-c]pyridin-4- yl)methyl)amino)acetamido)heptanamido)- 3,3-dimethylbutanoyl)-4-hydroxy- N-(4-(4-methylthiazol-5- yl)benzyl)pyrrolidine-2-carboxamide |
| 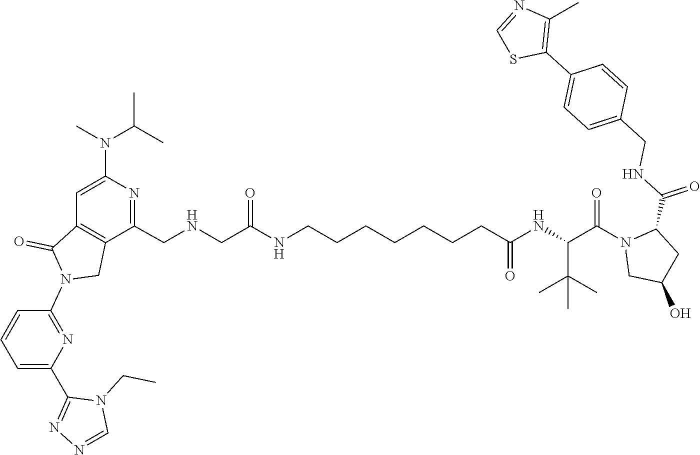 [SMILES: CCn1cnnc1-c1cccc(N2Cc3c(cc(N(C)C(C)C)nc3CNCC(=O)NCCCCCCCC(=O)N[C@H](C(=O)N3C[C@H](O)C[C@H]3C(=O)NCc3ccc(-c4scnc4C)cc3)C(C)(C)C)C2=O)n1] | (2S,4R)-1-((S)-2-(8-(2-(((2-(6-(4-ethyl- 4H-1,2,4-triazol-3-yl)pyridin-2-yl)-6- (isopropyl(methyl)amino)-1-oxo-2,3- dihydro-1H-pyrrolo[3,4-c]pyridin-4- yl)methyl)amino)acetamido)octanamido)- 3,3-dimethylbutanoyl)-4-hydroxy-N- (4-(4-methylthiazol-5- yl)benzyl)pyrrolidine-2-carboxamide |
| 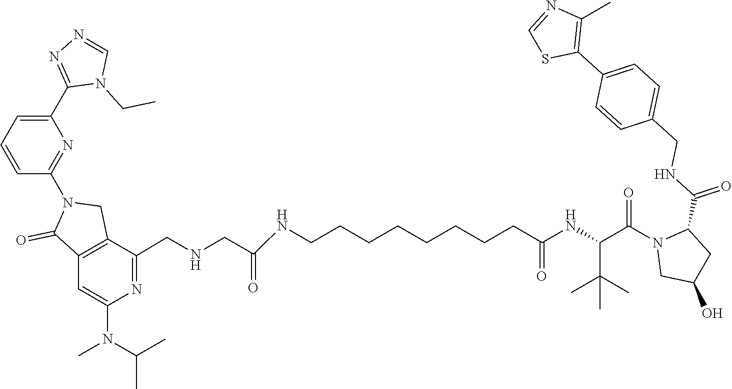 [SMILES: CCn1cnnc1-c1cccc(N2Cc3c(cc(N(C)C(C)C)nc3CNCC(=O)NCCCCCCCCC(=O)N[C@H](C(=O)N3C[C@H](O)C[C@H]3C(=O)NCc3ccc(-c4scnc4C)cc3)C(C)(C)C)C2=O)n1] | (2S,4R)-1-((S)-2-(9-(2-(((2-(6-(4-ethyl- 4H-1,2,4-triazol-3-yl)pyridin-2-yl)-6- (isopropyl(methyl)amino)-1-oxo-2,3- dihydro-1H-pyrrolo[3,4-c]pyridin-4- yl)methyl)amino)acetamido)nonanamido)- 3,3-dimethylbutanoyl)-4-hydroxy-N- (4-(4-methylthiazol-5- yl)benzyl)pyrrolidine-2-carboxamide |
| 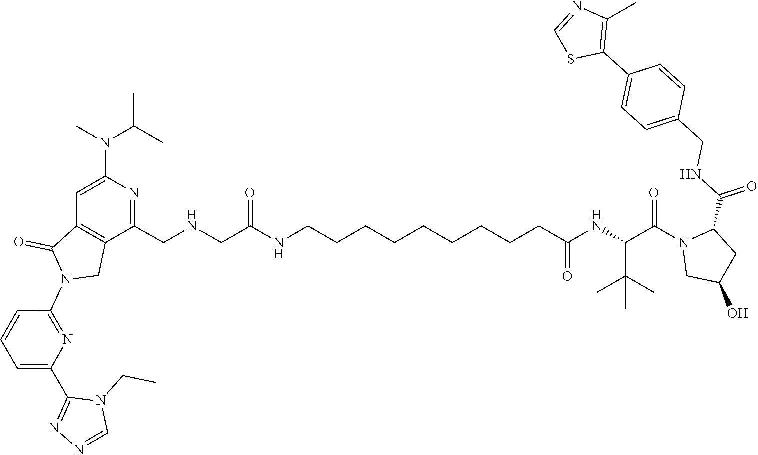 [SMILES: CCn1cnnc1-c1cccc(N2Cc3c(cc(N(C)C(C)C)nc3CNCC(=O)NCCCCCCCCCC(=O)N[C@H](C(=O)N3C[C@H](O)C[C@H]3C(=O)NCc3ccc(-c4scnc4C)cc3)C(C)(C)C)C2=O)n1] | (2S,4R)-1-((S)-2-(10-(2-(((2-(6-(4- ethyl-4H-1,2,4-triazol-3-yl)pyridin-2-yl)- 6-(isopropyl(methyl)amino)-1-oxo-2,3- dihydro-1H-pyrrolo[3,4-c]pyridin-4- yl)methyl)amino)acetamido)decanamido)- 3,3-dimethylbutanoyl)-4-hydroxy-N- (4-(4-methylthiazol-5- yl)benzyl)pyrrolidine-2-carboxamide |
| 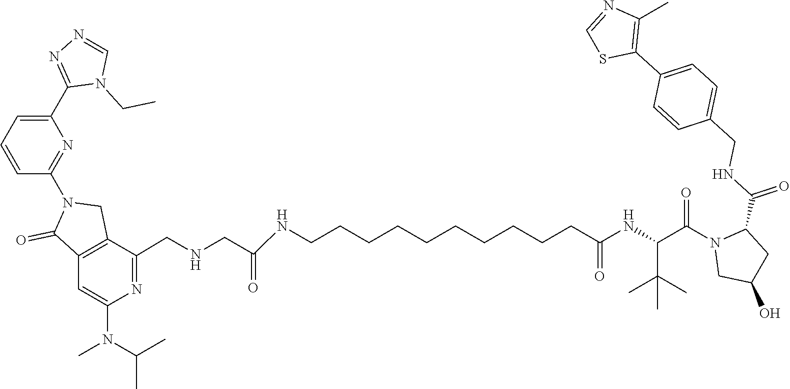 [SMILES: CCn1cnnc1-c1cccc(N2Cc3c(cc(N(C)C(C)C)nc3CNCC(=O)NCCCCCCCCCCC(=O)N[C@H](C(=O)N3C[C@H](O)C[C@H]3C(=O)NCc3ccc(-c4scnc4C)cc3)C(C)(C)C)C2=O)n1] | (2S,4R)-1-((S)-2-(11-(2-(((2-(6-(4- ethyl-4H-1,2,4-triazol-3-yl)pyridin-2-yl)- 6-(isopropyl(methyl)amino)-1-oxo-2,3- dihydro-1H-pyrrolo[3,4-c]pyridin-4- yl)methyl)amino)acetamido)undecanamido)- 3,3-dimethylbutanoyl)-4- hydroxy-N-(4-(4-methylthiazol-5- yl)benzyl)pyrrolidine-2-carboxamide |
| 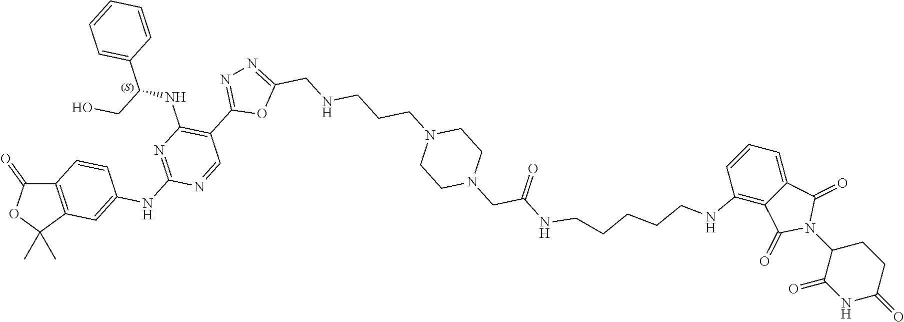 | 2-(4-(3-(((5-(2-((3,3-dimethyl-1-oxo- 1,3-dihydroisobenzofuran-5-yl)amino)- 4-(((S)-2-hydroxy-1- phenylethyl)amino)pyrimidin-5-yl)- 1,3,4-oxadiazol-2- yl)methyl)amino)propyl)piperazin-1-yl)- N-(5-((2-(2,6-dioxopiperidin-3-yl)-1,3- dioxoisoindolin-4- yl)amino)pentyl)acetamide |
| 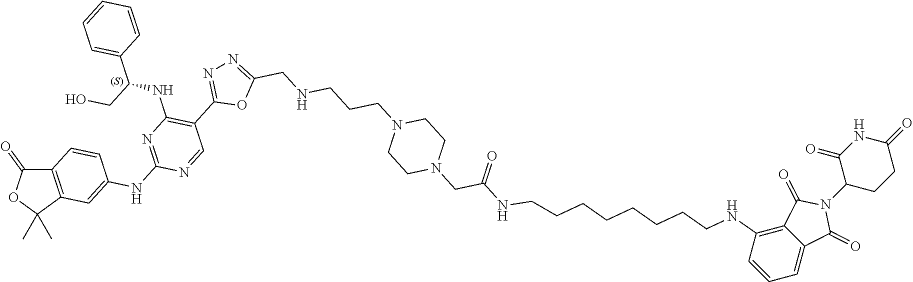 | 2-(4-(3-(((5-(2-((3,3-dimethyl-1-oxo- 1,3-dihydroisobenzofuran-5-yl)amino)- 4-(((S)-2-hydroxy-1- phenylethyl)amino)pyrimidin-5-yl)- 1,3,4-oxadiazol-2- yl)methyl)amino)propyl)piperazin-1-yl)- N-(8-((2-(2,6-dioxopiperidin-3-yl)-1,3- dioxoisoindolin-4- yl)amino)octyl)acetamide |
| 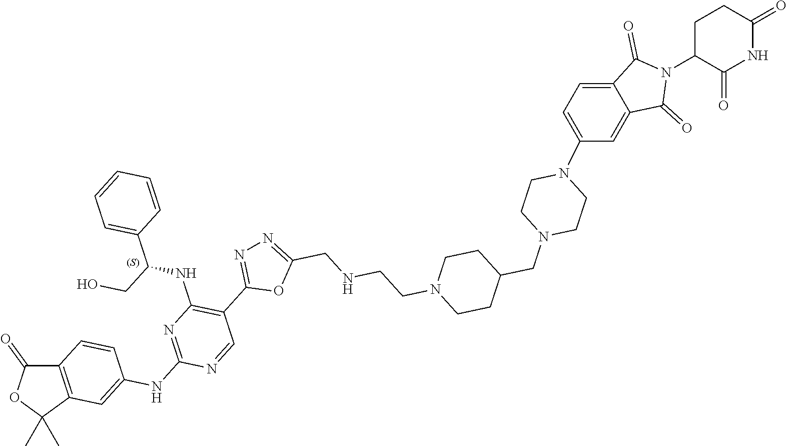 | 5-(4-((1-(2-(((5-(2-((3,3-dimethyl-1-oxo- 1,3-dihydroisobenzofuran-5-yl)amino)- 4-(((S)-2-hydroxy-1- phenylethyl)amino)pyrimidin-5-yl)- 1,3,4-oxadiazol-2- yl)methyl)amino)ethyl)piperidin-4- yl)methyl)piperazin-1-yl)-2-(2,6- dioxopiperidin-3-yl)isoindoline-1,3- dione |
| 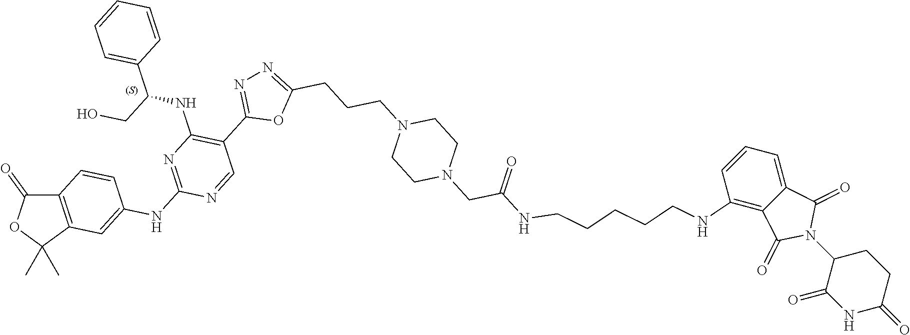 | 2-(4-(3-(5-(2-((3,3-dimethyl-1-oxo-1,3- dihydroisobenzofuran-5-yl)amino)-4- (((S)-2-hydroxy-1- phenylethyl)amino)pyrimidin-5-yl)- 1,3,4-oxadiazol-2-yl)propyl)piperazin- 1-yl)-N-(5-((2-(2,6-dioxopiperidin-3-yl)- 1,3-dioxoisoindolin-4- yl)amino)pentyl)acetamide |
| 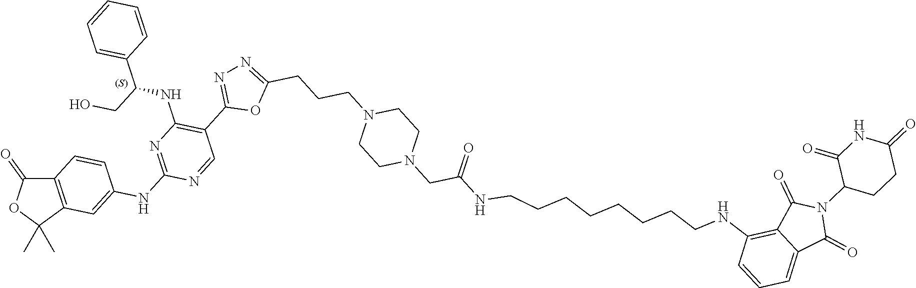 | 2-(4-(3-(5-(2-((3,3-dimethyl-1-oxo-1,3- dihydroisobenzofuran-5-yl)amino)-4- (((S)-2-hydroxy-1- phenylethyl)amino)pyrimidin-5-yl)- 1,3,4-oxadiazol-2-yl)propyl)piperazin- 1-yl)-N-(8-((2-(2,6-dioxopiperidin-3-yl)- 1,3-dioxoisoindolin-4- yl)amino)octyl)acetamide |
| 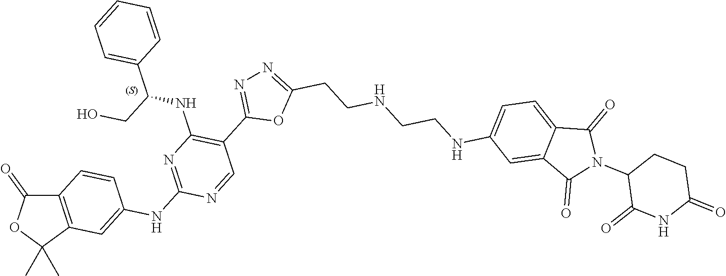 | 5-((2-((2-(5-(2-((3,3-dimethyl-1-oxo- 1,3-dihydroisobenzofuran-5-yl)amino)- 4-(((S)-2-hydroxy-1- phenylethyl)amino)pyrimidin-5-yl)- 1,3,4-oxadiazol-2- yl)ethyl)amino)ethyl)amino)-2-(2,6- dioxopiperidin-3-yl)isoindoline-1,3- dione |
| 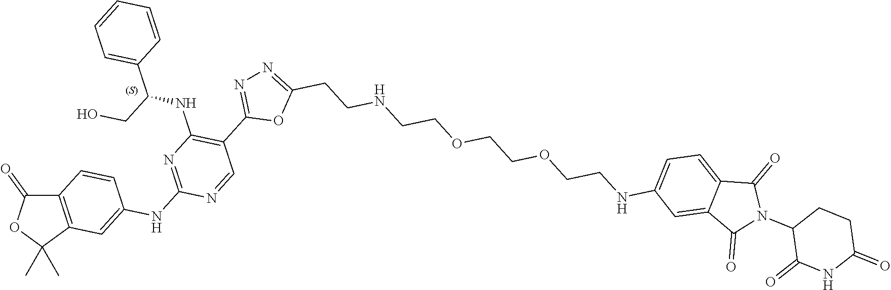 | 5-((2-(2-(2-((2-(5-(2-((3,3-dimethyl-1- oxo-1,3-dihydroisobenzofuran-5- yl)amino)-4-(((S)-2-hydroxy-1- phenylethyl)amino)pyrimidin-5-yl)- 1,3,4-oxadiazol-2- yl)ethyl)amino)ethoxy)ethoxy)ethyl) amino)-2-(2,6-dioxopiperidin-3- yl)isoindoline-1,3-dione |
| 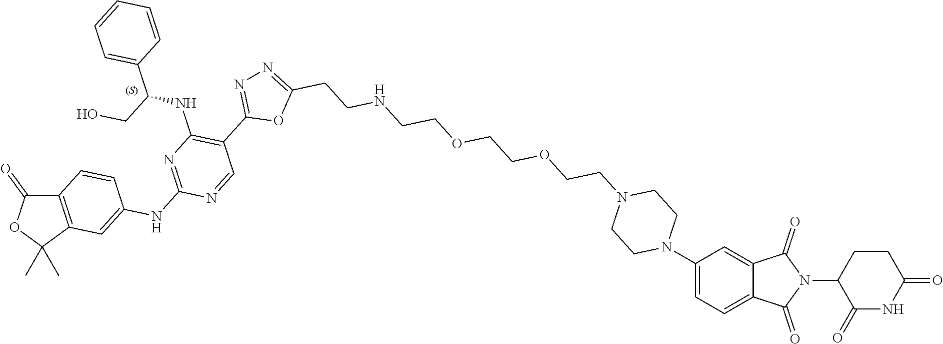 | 5-(4-(2-(2-(2-((2-(5-(2-((3,3-dimethyl-1- oxo-1,3-dihydroisobenzofuran-5- yl)amino)-4-(((S)-2-hydroxy-1- phenylethyl)amino)pyrimidin-5-yl)- 1,3,4-oxadiazol-2- yl)ethyl)amino)ethoxy)ethoxy)ethyl) piperazin-1-yl)-2-(2,6-dioxopiperidin-3- yl)isoindoline-1,3-dione |
| 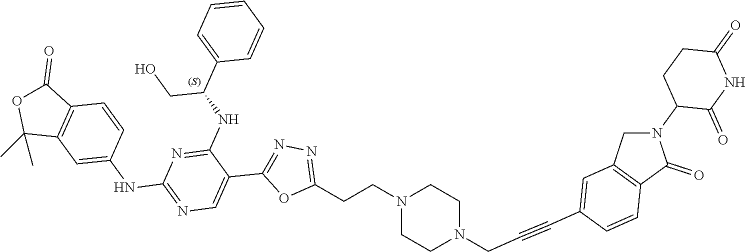 | 3-(5-(3-(4-(2-(5-(2-((3,3-dimethyl-1- oxo-1,3-dihydroisobenzofuran-5- yl)amino)-4-(((S)-2-hydroxy-1- phenylethyl)amino)pyrimidin-5-yl)- 1,3,4-oxadiazol-2-yl)ethyl)piperazin-1- yl)prop-1-yn-1-yl)-1-oxoisoindolin-2- yl)piperidine-2,6-dione |
| 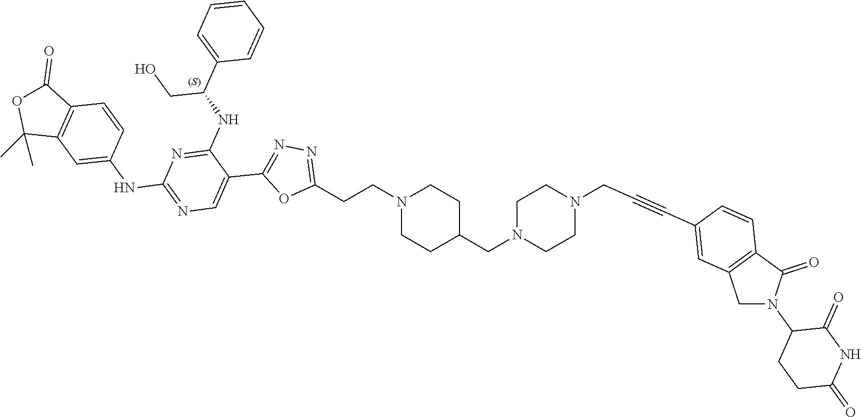 | 3-(5-(3-(4-((1-(2-(5-(2-((3,3-dimethyl-1- oxo-1,3-dihydroisobenzofuran-5- yl)amino)-4-(((S)-2-hydroxy-1- phenylethyl)amino)pyrimidin-5-yl)- 1,3,4-oxadiazol-2-yl)ethyl)piperidin-4- yl)methyl)piperazin-1-yl)prop-1-yn-1- yl)-1-oxoisoindolin-2-yl)piperidine-2,6- dione |
| 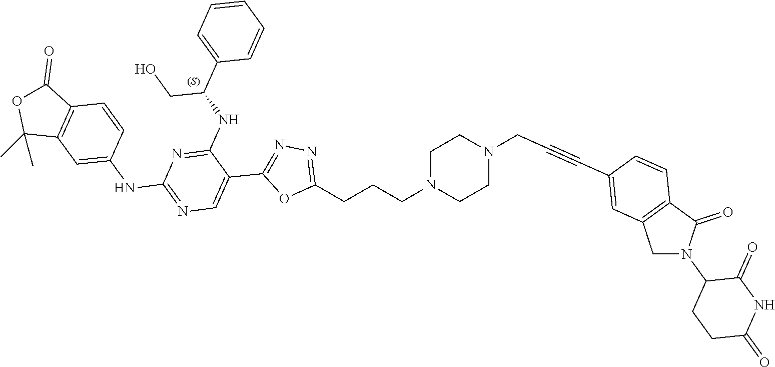 | 3-(5-(3-(4-(3-(5-(2-((3,3-dimethyl-1- oxo-1,3-dihydroisobenzofuran-5- yl)amino)-4-(((S)-2-hydroxy-1- phenylethyl)amino)pyrimidin-5-yl)- 1,3,4-oxadiazol-2-yl)propyl)piperazin- 1-yl)prop-1-yn-1-yl)-1-oxoisoindolin-2- yl)piperidine-2,6-dione |
| 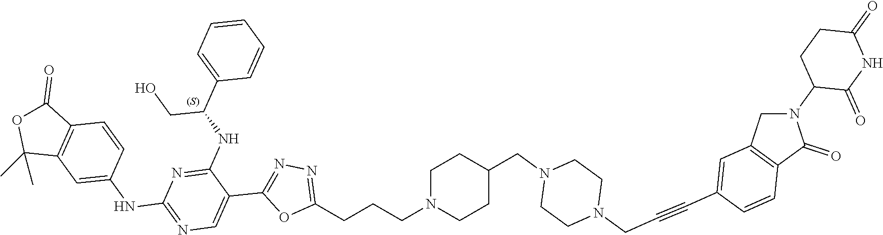 | 3-(5-(3-(4-((1-(3-(5-(2-((3,3-dimethyl-1- oxo-1,3-dihydroisobenzofuran-5- yl)amino)-4-(((S)-2-hydroxy-1- phenylethyl)amino)pyrimidin-5-yl)- 1,3,4-oxadiazol-2-yl)propyl)piperidin-4- yl)methyl)piperazin-1-yl)prop-1-yn-1- yl)-1-oxoisoindolin-2-yl)piperidine-2,6- dione |
| 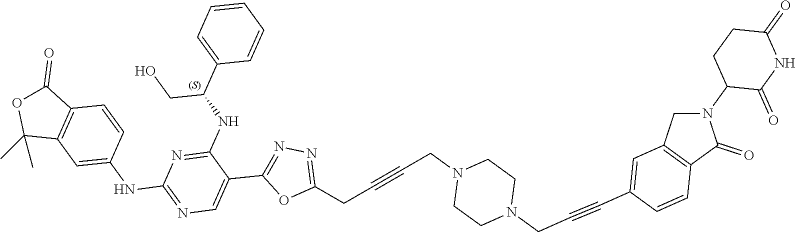 | 3-(5-(3-(4-(4-(5-(2-((3,3-dimethyl-1- oxo-1,3-dihydroisobenzofuran-5- yl)amino)-4-(((S)-2-hydroxy-1- phenylethyl)amino)pyrimidin-5-yl)- 1,3,4-oxadiazol-2-yl)but-2-yn-1- yl)piperazin-1-yl)prop-1-yn-1-yl)-1- oxoisoindolin-2-yl)piperidine-2,6-dione |
| 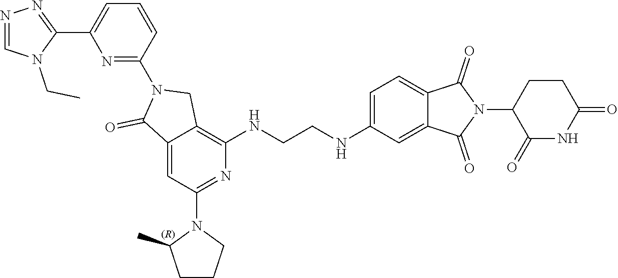 | 2-(2,6-dioxopiperidin-3-yl)-5-((2-((2-(6- (4-ethyl-4H-1,2,4-triazol-3-yl)pyridin-2- yl)-6-((R)-2-methylpyrrolidin-1-yl)-1- oxo-2,3-dihydro-1H-pyrrolo[3,4- c]pyridin-4- yl)amino)ethyl)amino)isoindoline-1,3- dione |
| 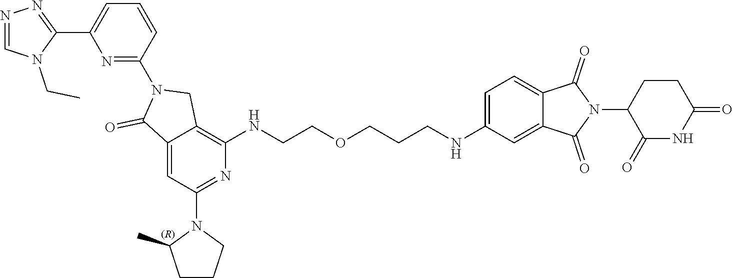 | 2-(2,6-dioxopiperidin-3-yl)-5-((3-(2-((2- (6-(4-ethyl-4H-1,2,4-triazol-3-yl)pyridin- 2-yl)-6-((R)-2-methylpyrrolidin-1-yl)-1- oxo-2,3-dihydro-1H-pyrrolo[3,4- c]pyridin-4- yl)amino)ethoxy)propyl)amino) isoindoline-1,3-dione |
| 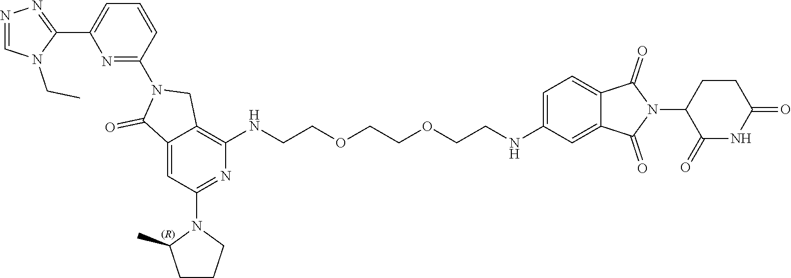 | 2-(2,6-dioxopiperidin-3-yl)-5-((2-(2-(2- ((2-(6-(4-ethyl-4H-1,2,4-triazol-3- yl)pyridin-2-yl)-6-((R)-2- methylpyrrolidin-1-yl)-1-oxo-2,3- dihydro-1H-pyrrolo[3,4-c]pyridin-4- yl)amino)ethoxy)ethoxy)ethyl)amino) isoindoline-1,3-dione |
| 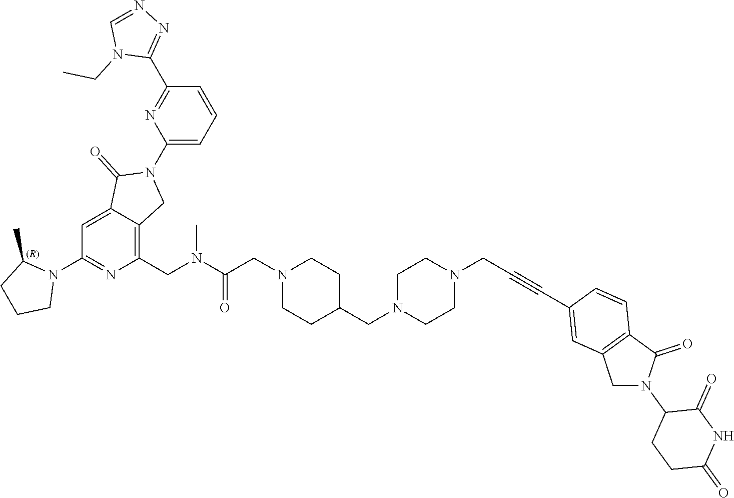 | 2-(4-((4-(3-(2-(2,6-dioxopiperidin-3-yl)- 1-oxoisoindolin-5-yl)prop-2-yn-1- yl)piperazin-1-yl)methyl)piperidin-1-yl)- N-((2-(6-(4-ethyl-4H-1,2,4-triazol-3- yl)pyridin-2-yl)-6-((R)-2- methylpyrrolidin-1-yl)-1-oxo-2,3- dihydro-1H-pyrrolo[3,4-c]pyridin-4- yl)methyl)-N-methylacetamide |
| 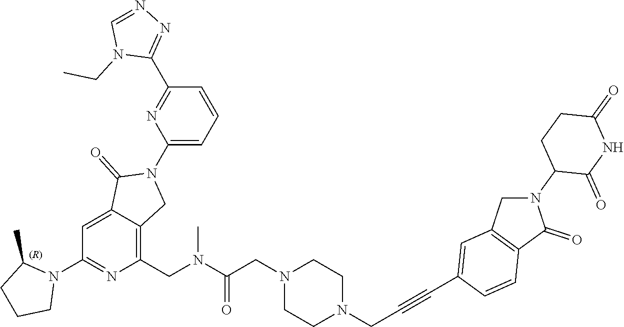 | 2-(4-(3-(2-(2,6-dioxopiperidin-3-yl)-1- oxoisoindolin-5-yl)prop-2-yn-1- yl)piperazin-1-yl)-N-((2-(6-(4-ethyl-4H- 1,2,4-triazol-3-yl)pyridin-2-yl)-6-((R)-2- methylpyrrolidin-1-yl)-1-oxo-2,3- dihydro-1H-pyrrolo[3,4-c]pyridin-4- yl)methyl)-N-methylacetamide |
| 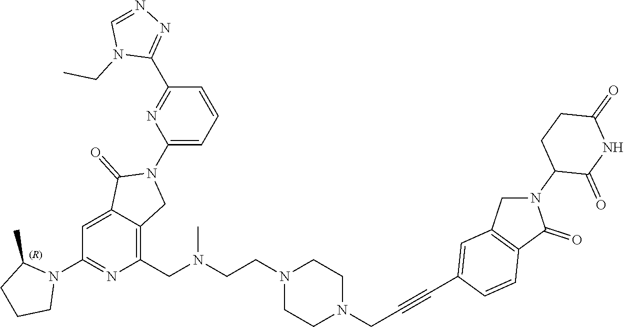 | 3-(5-(3-(4-(2-(((2-(6-(4-ethyl-4H-1,2,4- triazol-3-yl)pyridin-2-yl)-6-((R)-2- methylpyrrolidin-1-yl)-1-oxo-2,3- dihydro-1H-pyrrolo[3,4-c]pyridin-4- yl)methyl)(methyl)amino)ethyl) piperazin-1-yl)prop-1-yn-1-yl)-1- oxoisoindolin-2-yl)piperidine-2,6-dione |
| 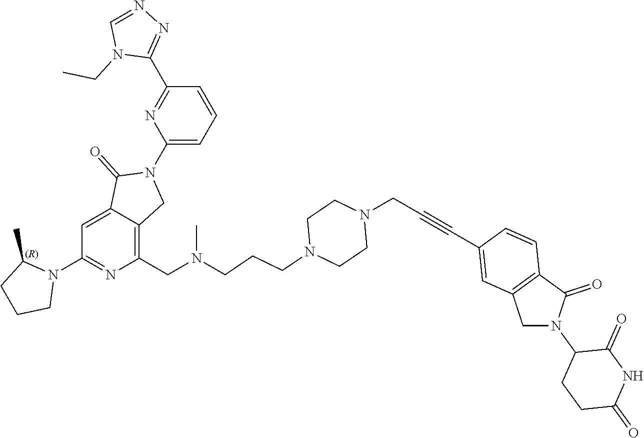 | 3-(5-(3-(4-(3-(((2-(6-(4-ethyl-4H-1,2,4- triazol-3-yl)pyridin-2-yl)-6-((R)-2- methylpyrrolidin-1-yl)-1-oxo-2,3- dihydro-1H-pyrrolo[3,4-c]pyridin-4- yl)methyl(methyl)amino)propyl) piperazin-1-yl)prop-1-yn-1-yl)-1- oxoisoindolin-2-yl)piperidine-2,6-dione |
| 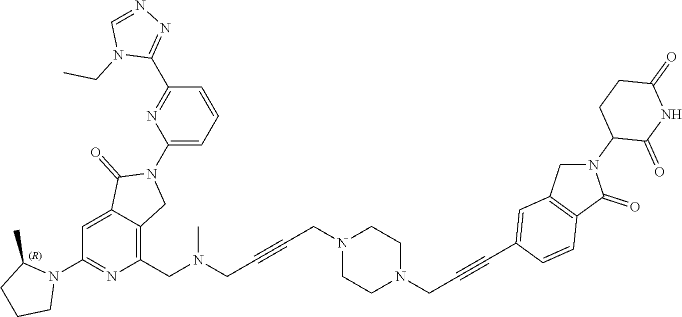 | 3-(5-(3-(4-(4-(((2-(6-(4-ethyl-4H-1,2,4- triazol-3-yl)pyridin-2-yl)-6-((R)-2- methylpyrrolidin-1-yl)-1-oxo-2,3- dihydro-1H-pyrrolo[3,4-c]pyridin-4- yl)methyl)(methyl)amino)but-2-yn-1- yl)piperazin-1-yl)prop-1-yn-1-yl)-1- oxoisoindolin-2-yl)piperidine-2,6-dione |

In Table 1 and Table 2, the left portion of the structure of the HPK1 disruptors/degraders binds to HPK1 and the right portion of the structure recruits the ubiquitination machinery to HPK1, which induces poly-ubiquitination and degradation of HPK1 at the proteasome.

Compounds corresponding to the Examples 1-79 have been synthesized and the final compounds provided with a Compound ID. Compounds in Table 2 have not been synthesized and are not provided with a compound code. These compounds may be synthesized according to the schemes set forth above.

As used herein, in case of discrepancy between the structure and chemical name provided for a particular compound, the given structure shall control.

### Example 80

### General Chemistry Methods

General method for LCMS: Mobile phase: Ramp from 5% ACN (0.01875% TFA) in water (0.0375% TFA) to 95% ACN in water in 0.60 min, flow rate is set at 2.0 mL/min; then hold at 95% ACN for 0.18 min, flow rate is set at 2.0 mL/min; return back to 5% ACN in water and hold for 0.02 min, flow rate is set at 2.0 mL/min. Column temperature at 50° C. The column is of Kinetex® EVO C18, 2.1×30 mm, 5 m.

General method for NMR: ¹H NMR spectra were recorded using Bruker 400 MHz instrument using CDCl₃ or DMSO-d₆ as solvent with tetramethylsilane (TMS) as an internal standard. Chemical shifts were reported in parts per million (ppm) relative to TMS (6). Multiplicities were indicated by s (singlet), d (doublet), t (triplet), q (quartet), m (multiplet), coupling constant J was reported in hertz (Hz).

### Example 81

### Biological Methods:

TCR-Induced IL-2 Production by Jurkat T Cells Jurkat cell line, Clone E6-1 (https://www.atcc.org/products/tib-152), is used as a model system to assess T cell activation in response to exposure to HPK1 PROTACS. Jurkat is easy to propagate indefinitely, and rapidly in large quantities. Jurkat secretes IL-2 and other cytokines upon antibody-mediated crosslinking of the T cell antigen receptor (TCR) in conjunction with the co-engagement of its co-receptor CD28. The presence of IL-2 in the supernatant can be easily assessed and quantitated using commercially available human IIL-2 ELISA assay. IL-2 production is a. well-accepted proxy readout for the activation state of Jurkat since these cells produce IL-2 only in response to the co-engagement of TCR and CD28. Jurkat endogenously expresses a readily detectable level of HPK1. CRISPR-mediated gene editing of the HPK1 loci has shown to impart Jurkat with hyper-activated T cell phenotype, phenocopying the phenotypes observed in genetically engineered HPK1−/− mice. For these reasons, Jurkat is an ideal model system to screen for HPK1 PROTACS leads.

Jurkat is grown in the “complete” RPMI-1640 medium, which is comprised of [RPMI-1640 (Gibco Cat. #: 11875-093), 10% FCS (Gibco Cat. #: A38400-01), 1×Pen/Strep/L-glutamine (Corning Cat. #: 30-009-CI).] Cells in the log phase of growth (approximately 2.5×10⁵ cells per m L) are used for the studies.

Twenty-four hours before the start of the stimulation procedure, coat sterile tissue culture-treated, flat bottom 96-well plate (Corning Cat. #: 3596) with 50 μL of 10 ug/ml of anti-CD3e mAb, clone OKT3 (Invitrogen Cat. #: 16-0037-85), in PBS solution (Gibco Cat. #: 10010-023). On the same day, Jurkat cells are exposed to a series of 3-fold dilutions of the investigational HPK1 PROTACS or its solvent control, DMSO, starting at 1 μM, in 200 IL of complete RPMI media at 1×10⁵ cells per well. After the exposure to HPK1 compounds for 24 hours, cells are transferred to the washed CD3e-coated plates (2× washes with PBS), in presence of 1 μg of CD28 antibody per well (Invitrogen Cat. #: 16-0289-85). The binding of these two monoclonal antibodies initiates the T cells activation process. Cells were left under this stimulation in 37C, 5% CO2 incubator for 24 hours before supernatants are harvested for assessment by IL-2 ELISA (Invitrogen, Catalog #88-7025-88). Supernatants are diluted 1:10 with ELISA binding buffer provided by the kit's manufacturer so that the IL-2 levels would fall within the dynamic range of the assay.

### HPK1 Degradation

To assess the degradation of HPK1, some compounds that induced an elevated level of IL-2 production to a level higher at least 2 times the level produces by cells that were treated by DMSO alone, assessed for their ability to degrade endogenous HPK1. Jurkat cells are treated with a series of three-fold dilutions of HPK1 hits, starting at 1 μg for 24 hours. Cells are harvested and lysed in Thermo Scientific™ M-PER™ Mammalian Protein Extraction Reagent (50 μL per 2×10⁶ cells). Protein concentrations in lysates are determined by BCA assay (Pierce™ BCA Protein Assay Kit Catalog number: 23227) and 50 μg of proteins are resolved by SDS-PAGE. PVDF membranes containing resolved proteins are blocked by BSA and probed with anti-HPK1 pAb (Cell Signaling Technologies, HPK1 Antibody #4472) at 1 μg/mL concentration, using a standard Western blot procedure.

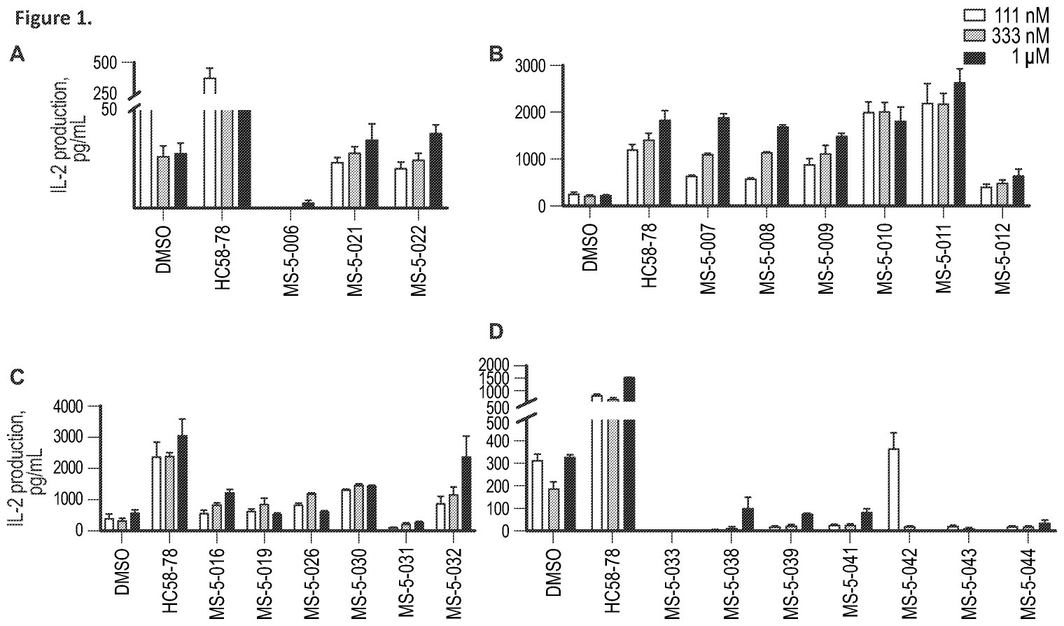

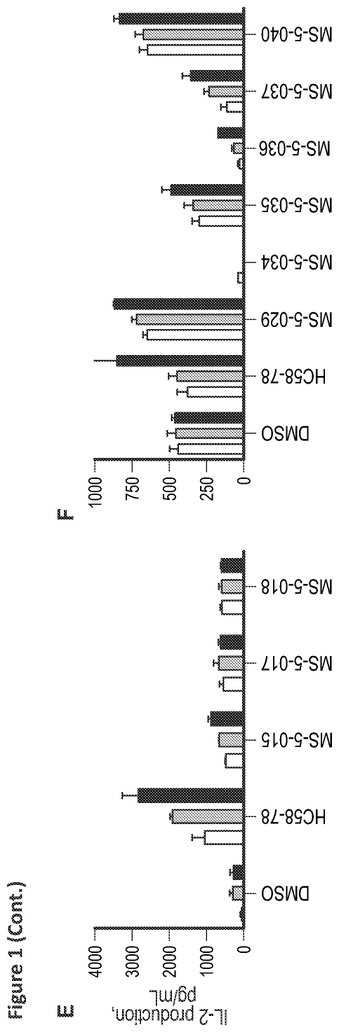

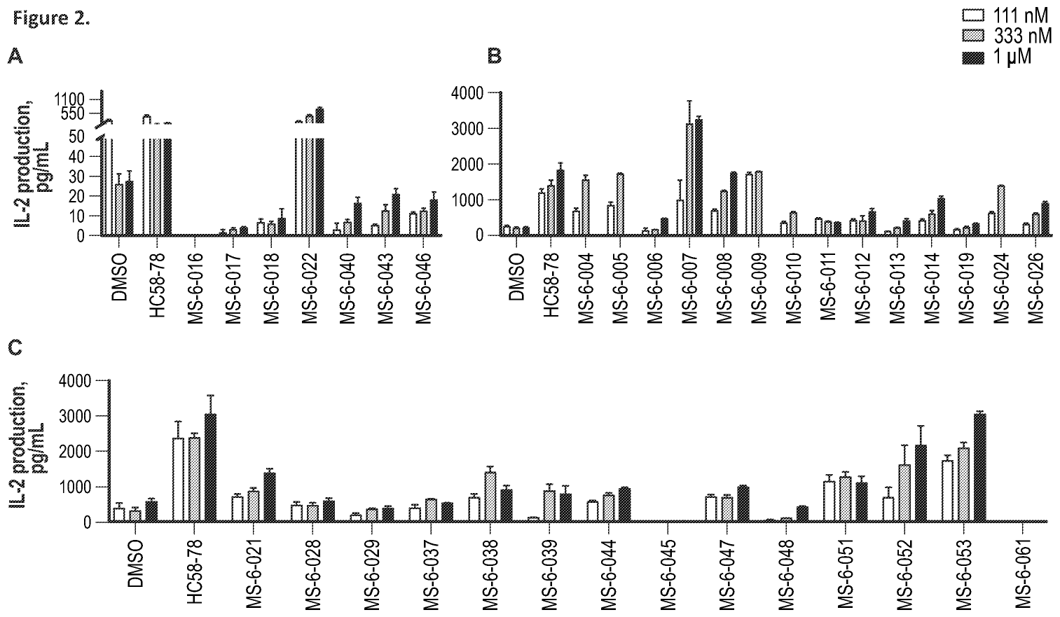

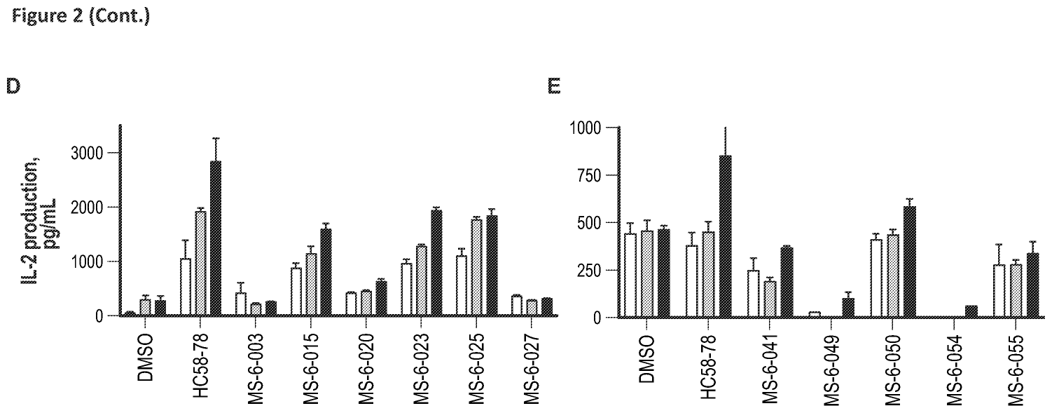

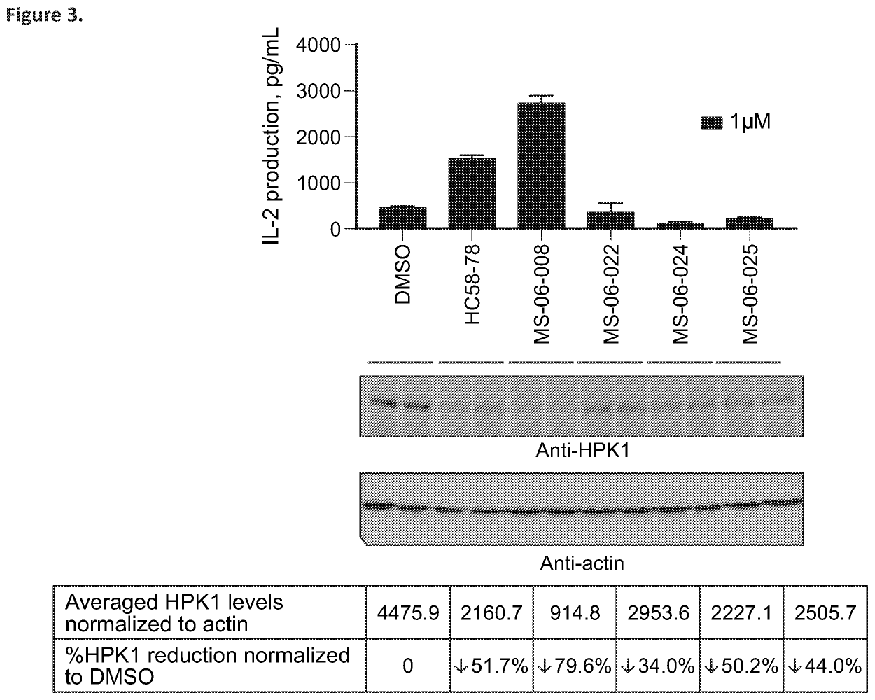

The effect of synthesized compounds on the TCR-induced IL-2 production by Jurkat T cells and the effect of select compound on the HPK1 expression level in Jurkat T cells are shown in FIGS. 1A-1F; 2A-2D and 3.

### OTHER ASPECTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

### REFERENCES

Alzabin, S., Bhardwaj, N., Kiefer, F., Sawasdikosol, S., and Burakoff, S. (2009). Hematopoietic progenitor kinase 1 is a negative regulator of dendritic cell activation. J Immunol 182, 6187-6194.
Alzabin, S., Pyarajan, S., Yee, H., Kiefer, F., Suzuki, A., Burakoff, S., and Sawasdikosol, S. (2010). Hematopoietic progenitor kinase 1 is a critical component of prostaglandin E2-mediated suppression of the anti-tumor immune response. Cancer Immunol Immunother 59, 419-429.
Bondeson, D. P., Mares, A., Smith, I. E., Ko, E., Campos, S., Miah, A. H., Mulholland, K. E., Routly, N., Buckley, D. L., Gustafson, J. L., et al. (2015). Catalytic in vivo protein knockdown by small-molecule PROTACs. Nat Chem Biol 11, 611-617.
Buckley, D. L., and Crews, C. M. (2014). Small-molecule control of intracellular protein levels through modulation of the ubiquitin proteasome system. Angew Chem Int Ed Engl 53, 2312-2330.
Buckley, D. L., Gustafson, J. L., Van Molle, I., Roth, A. G., Tae, H. S., Gareiss, P. C., Jorgensen, W. L., Ciulli, A., and Crews, C. M. (2012a). Small-molecule inhibitors of the interaction between the E3 ligase VHL and HIFlalpha. Angew Chem Int Ed Engl 51, 11463-11467.
Buckley, D. L., Raina, K., Darricarrere, N., Hines, J., Gustafson, J. L., Smith, I. E., Miah, A. H., Harling, J. D., and Crews, C. M. (2015). HaloPROTACS: Use of Small Molecule PROTACs to Induce Degradation of HaloTag Fusion Proteins. ACS Chem Biol 10, 1831-1837.
Buckley, D. L., Van Molle, I., Gareiss, P. C., Tae, H. S., Michel, J., Noblin, D. J., Jorgensen, W. L., Ciulli, A., and Crews, C. M. (2012b). Targeting the von Hippel-Lindau E3 ubiquitin ligase using small molecules to disrupt the VHL/HIF-lalpha interaction. J Am Chem Soc 134, 4465-4468.
Burnet, F. M. (1970). The concept of immunological surveillance. Progress in experimental tumor research 13, 1-27.
Chamberlain, P. P., Lopez-Girona, A., Miller, K., Carmel, G., Pagarigan, B., Chie-Leon, B., Rychak, E., Corral, L. G., Ren, Y. J., Wang, M., et al. (2014). Structure of the human Cereblon-DDB1-lenalidomide complex reveals basis for responsiveness to thalidomide analogs. Nat Struct Mol Biol 21, 803-809.
Corthay, A. (2014). Does the immune system naturally protect against cancer? Frontiers in immunology 5, 197.
Davies, T. G., Wixted, W. E., Coyle, IE., Griffiths-Jones, C., Heam, K., Mcl\fonamin, R., Norton, D., Rich, S. J., Richardson, C., Saxty, G., et al. (2016). Monoacidic Inhibitors of the Kelch-like ECH-Associated Protein 1: Nuclear Factor Erythroid 2-Related Factor 2 (KEAP1:NRF2) Protein-Protein Interaction with High Cell Potency Identified by Fragment-Based Discovery. J Med Chem 59, 3991-4006.
Fischer, E. S., Bohm, K., Lydeard, J. R., Yang, H., Stadler, M. B., Cavadini, S., Nagel, J., Serluca, F., Acker, V., Lingaraju, G. M., et al. (2014). Structure of the DDB1-CRBN E3 ubiquitin ligase in complex with thalidomide. Nature 512, 49-53.
Galdeano, C., Gadd, M. S., Soares, P., Scaffidi, S., Van Molle, I., Birced, I., Hewitt, S., Dias, D. M., and Ciulli, A. (2014). Structure-guided design and optimization of small molecules targeting the protein-protein interaction between the von Hippel-Lindau (VHL) E3 ubiquitin ligase and the hypoxia inducible factor (HIF) alpha subunit with in vitro nanomolar affinities. J Med Chem 57, 8657-8663.
Gillis, S., and J. Watson. 1980. Biochemical and biological characterization of lymphocyte regulatory molecules. V. Identification of an interleukin 2-producing human leukemia T cell line. The Journal of experimental medicine 152: 1709-1719.
Hernandez, S., Qing, J., Thibodeau, R. H., Du, X., Park, S., Lee, H. M., Xu, M., Oh, S., Navarro, A., Roose-Girma, M., et al. (2018). The Kinase Activity of Hematopoietic Progenitor Kinase 1 Is Essential for the Regulation of T Cell Function. Cell reports 25, 80-94.
Hu, M. C., Qiu, W. R., Wang, X., Meyer, C. F., and Tan, T. H. (1996). Human HPK1, a novel human hematopoietic progenitor kinase that activates the JNK/SAPK kinase cascade. Genes Dev 10, 2251-2264.
Ito, T., Ando, H., Suzuki, T., Ogura, T., Hotta, K., Imamura, Y., Yamaguchi, Y., and Handa, H. (2010). Identification of a primary target of thalidomide teratogenicity. Science 327, 1345-1350.
Kiefer, F., Tibbles, L. A., Anafi, M., Janssen, A., Zanke, B. W., Lassam, N., Pawson, T., Woodgett, J. R., and Iscove, N. N. (1996). HPK1, a hematopoietic protein kinase activating the SAPK/JNK pathway. EMBO J 15, 7013-7025.
Lai, A. C., Toure, M., Hellerschmied, D., Salami, J., Jaime-Figueroa, S., Ko, E., Hines, J., and Crews, C. M. (2016). Modular PROTAC Design for the Degradation of Oncogenic BCR-ABL. Angew Chem Int Ed Engl 55, 807-810.
Ling, P., Meyer, C. F., Redmond, L. P., Shui, J. W., Davis, B., Rich, R. R., Hu, M. C., Wange, R. L., and Tan, T. H. (2001). Involvement of hematopoietic progenitor kinase 1 in T cell receptor signaling. The Journal of biological chemistry 276, 18908-18914.
Liou, J., Kiefer, F., Dang, A., Hashimoto, A., Cobb, M. H., Kurosaki, T., and Weiss, A. (2000). HPK1 is activated by lymphocyte antigen receptors and negatively regulates AP-1. Immunity 12, 399-408.
Liu, J., Curtin, J., You, D., Hillerman, S., Li-Wang, B., Eraslan, R., Xie, J., Swanson, J., Ho, C. P., Oppenheimer, S., et al. (2019). Critical role of kinase activity of hematopoietic progenitor kinase 1 in anti-tumor immune surveillance. PloS one 14, e0212670.
Lu, J., Qian, Y., Altieri, M., Dong, H., Wang, J., Raina, K., Hines, J., Winkler, J. D., Crew, A. P., Coleman, K., et al. (2015). Hijacking the E3 Ubiquitin Ligase Cereblon to Efficiently Target BRD4. Chemistry & biology 22, 755-763.
Ohoka, N, Okuhira, K., Ito, M, Nagai, K, Shibata, N., Hattori, T., Ujikawa, O, Shimokawa, K., Sano, O., Koyama, R., et al (2017). In Vivo Knockdown of Pathogenic Proteins via Specific and Nongenetic Inhibitor of Apoptosis Protein (IAP)-dependent Protein Erasers (SNIPERs). J Biol Chem 292, 4556-4570.
Okuhira, K., Ohoka, N., Sai, K., Nishimaki-Mogami, T., Itoh, Y., Ishikawa, M., Hashimoto, Y., and Naito, M. (2011). Specific degradation of CRABP-II via cIAP1-mediated ubiquitylationinduced by hybrid molecules that crosslink c1AP1 and the target protein. FEBS Lett 585, 1147-1152.
Ribas, A., and Wolchok, J. D. (2018). Cancer immunotherapy using checkpoint blockade. Science (New York, NY 359, 1350-1355.
Sawasdikosol, S., Pyarajan, S., Alzabin, S., Matejovic, G., and Burakoff, S. J. (2007). Prostaglandin E2 activates HPK1 kinase activity via a PKA-dependent pathway. The Journal of biological chemistry 282, 34693-34699.
Sawasdikosol, S., Russo, K. M., and Burakoff, S. J. (2003). Hematopoietic progenitor kinase 1 (HPK1) regulates prostaglandin E2-induced fos gene transcription. Blood 101, 3687-3689.
Sawasdikosol, S., Zha, R., Yang, B., and Burakoff, S. (2012). HPK1 as a novel target for cancer immunotherapy. Immunologic research 54, 262-265.
Shibata, N., Miyamoto, N., Nagai, K., Shimokawa, K., Sameshima, T., Ohoka, N., Hattori, T., Imaeda, Y, Nara, H., Cho, N., et al. (2017). Development of protein degradation inducers of oncogenic BCR-ABL protein by conjugation of ABL kinase inhibitors and IAP ligands. Cancer Sci 108, 1657-1666.
Shui, J. W., Boomer, J. S., Han, J., Xu, J., Dement, G. A., Zhou, G., and Tan, T. H. (2007). Hematopoietic progenitor kinase 1 negatively regulates T cell receptor signaling and T cell-mediated immune responses. Nat Immunol 8, 84-91.
Sun, D., Li, Z, Rew, Y., Gribble, M, Bartberger, M. D., Beck, H. P., Canon, J., Chen, A., Chen, X, Chow, D., et al. (2014). Discovery of AMG 232, a potent, selective, and orally bioavailable MDM2-p53 inhibitor in clinical development. J Med Chem 57, 1454-1472.
Weiss, A., R. L. Wiskocil, and J. D. Stobo. 1984. The role of T3 surface molecules in the activation of human T cells: a two-stimulus requirement for IL 2 production reflects events occurring at a pre-translational level. J Immunol 133: 123-128.
Winter, G. E., Buckley, D. L., Paulk, J., Roberts, J. M., Souza, A., Dhe-Paganon, S., and Bradner, J. E. (2015). Phthalimide conjugation as a strategy for in vivo target protein degradation. Science 348, 1376-1381.
Xie, T., Lim, S. M., Westover, K. D., Dodge, M. E., Ercan, D., Ficarro, S. B., Udayakumar, D., Gurbani, D., Tae, H. S., Riddle, S. M., et al. (2014). Pharmacological targeting of the pseudokinase Her3. Nat Chem Biol 10, 1006-1012.
Zengerle, M., Chan, K. H., and Ciulli, A. (2015). Selective Small Molecule Induced Degradation of the BET Bromodomain Protein BRD4. ACS Chem Biol 10, 1770-1777.

1. A bivalent compound comprising an HPK1 ligand conjugated to a degradation/disruption tag through a linker, the HPK1 ligand selected from the group consisting of:
a moiety according to FORMULA 1: 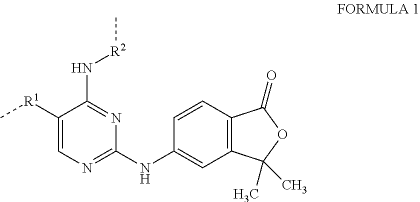
wherein
the “Linker” moiety of the bivalent compound is attached independently to R^{t} or R² (indicated by dotted line);
when the “Linker” moiety of the bivalent compound is attached to R²;
R¹ is, at each occurrence, independently selected from hydrogen, halogen, oxo, CN, NO₂, OR⁴, SR⁴, NR⁴R⁵, C(O)R⁴, C(O)OR⁴, C(O)NR⁴R⁵, S(O)R⁴, S(O)₂R⁴, S(O)₂NR⁴R⁵, NR⁶C(O)OR⁴, NR⁶C(O)R⁴, NR⁶C(O)NR⁴R⁵, NR⁶S(O)R⁴, NR⁶S(O)₂R⁴, NR⁶S(O)₂NR⁴R⁵, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl; wherein
R⁴, R⁵, and R⁶ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl; or
R⁴ and R⁵, R⁴ and R⁶, R⁵ and R⁶ together with the atom to which they are connected form an optionally substituted 3-20 membered cycloalkyl or heterocyclyl ring;
when the “Linker” moiety of the bivalent compound is attached to R¹;
R¹ is, at each occurrence, independently selected from OR⁴, SR⁴, NR⁴R⁵, C(O)R⁴, C(O)OR⁴, C(O)NR⁴R⁵, S(O)R⁴, S(O)₂R⁴, S(O)₂NR⁴R⁵, NR⁶C(O)OR⁴, NR⁶C(O)R⁴, NR⁶C(O)NR⁴R⁵, NR⁶S(O)R⁴, NR⁶S(O)₂R⁴, NR⁶S(O)₂NR⁴R⁵, C₁-C₈ alkylene, optionally substituted C₁-C₈ alkylene, optionally substituted C₂-C₈ alkenylene, optionally substituted C₂-C₈ alkynylene, optionally substituted C₁-C₈ alkoxylene, optionally substituted C₁-C₈alkoxyC₁-C₈alkylene, optionally substituted C₁-C₈alkylaminoC₁-C₈alkylene, optionally substituted 3-8 membered cycloalkylene, optionally substituted C₃-C₈ cycloalkoxylene, optionally substituted 3-8 membered heterocyclylene, optionally substituted arylene, and optionally substituted heteroarylene; wherein
R⁴ is null, or a bivalent moiety selected from optionally substituted C₁-C₈ alkylene, optionally substituted C₂-C₈ alkenylene, optionally substituted C₂-C₈ alkynylene, optionally substituted C₃-C₈ cycloalkylene, optionally substituted 3-8 membered heterocyclylene, optionally substituted arylene, and optionally substituted heteroarylene;
R⁵ and R⁶ are independently selected from optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₈ cycloalkyl, optionally substituted 3-8 membered heterocyclyl, optionally substituted C₂-C₈ alkenyl, optionally substituted C₂-C₈ alkynyl, optionally substituted aryl, and optionally substituted heteroaryl; or
R⁴ and R⁵, R⁴ and R⁶, R⁵ and R⁶ together with the atom to which they are connected form a 3-20 membered cycloalkyl or heterocyclyl ring;
when the “Linker” moiety of the bivalent compound is attached to R¹;
R² is, at each occurrence, independently selected from null, hydrogen, C(O)R⁷, C(O)OR⁷, C(O)NR⁷R⁸, S(O)R⁷, S(O)₂R⁷, S(O)₂NR⁷R′, NR⁹C(O)OR⁷, NR⁹C(O)R⁷, NR⁹C(O)NR⁷R⁸, NR⁹S(O)R⁷, NR⁹S(O)₂R⁷, NR⁹S(O)₂NR⁷R⁸, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl; wherein
R⁷, R⁸, and R⁹ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl; or
R⁷ and R⁸, R⁷ and R⁹, R⁸ and R⁹ together with the atom to which they are connected form an optionally substituted 3-20 membered cycloalkyl or heterocyclyl ring;
when the “Linker” moiety of the bivalent compound is attached to R²;
R² is, at each occurrence, independently selected from C(O)R⁷, C(O)OR⁷, C(O)NR⁷R⁸, S(O)R⁷, S(O)₂R⁷, S(O)₂NR⁷R′, NR⁹C(O)OR⁷, NR⁹C(O)R⁷, NR⁹C(O)NR⁷R⁸, NR⁹S(O)R⁷, NR⁹S(O)₂R⁷, NR⁹S(O)₂NR⁷R⁸, optionally substituted C₁-C₈ alkylene, optionally substituted C₂-C₈ alkenylene, optionally substituted C₂-C₈ alkynylene, optionally substituted C₁-C₈ alkoxylene, optionally substituted C₁-C₈alkoxyC₁-C₈alkylene, optionally substituted C₁-C₈alkylaminoC₁-C₈alkylene, optionally substituted 3-8 membered cycloalkylene, optionally substituted C₃-C₈ cycloalkoxylene, optionally substituted 3-8 membered heterocyclylene, optionally substituted arylene, and optionally substituted heteroarylene; wherein
R⁷ is null, or a bivalent moiety selected from optionally substituted C₁-C₈ alkylene, optionally substituted C₂-C₈ alkenylene, optionally substituted C₂-C₈ alkynylene, optionally substituted C₃-C₈ cycloalkylene, optionally substituted 3-8 membered heterocyclylene, optionally substituted arylene, and optionally substituted heteroarylene;
R⁸ and R⁹ are independently selected from optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₈ cycloalkyl, optionally substituted 3-8 membered heterocyclyl, optionally substituted C₂-C₈ alkenyl, optionally substituted C₂-C₈ alkynyl, optionally substituted aryl, and optionally substituted heteroaryl; or
R⁷ and R⁸, R⁷ and R⁹, R⁸ and R⁹ together with the atom to which they are connected form a 3-20 membered cycloalkyl or heterocyclyl ring;
a moiety according to FORMULA 2: 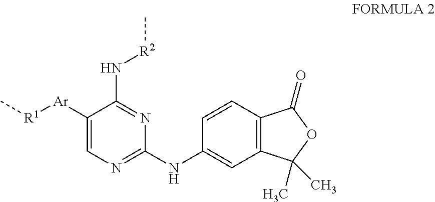
wherein
the “Linker” moiety of the bivalent compound is attached independently to R¹ or R² (indicated by dotted line);
the definitions of, R¹ and R², are the same as for FORMULA 1;
Ar is, at each occurrence, independently selected from null, aryl or heteroaryl, each of which is substituted with R¹ and optionally substituted with one or more substituents independently selected from hydrogen, halogen, oxo, CN, NO₂, OR¹², SR¹², NR¹²R¹³, OCOR¹², OCO₂R¹², OCONR¹²R¹³, COR¹², CO₂R¹², CONR¹²R¹³, SOR¹², SO₂R¹², SO₂NR¹²R¹³, NR¹⁴CO₂R¹², NR¹⁴COR¹², NR¹⁴C(O)NR¹²R¹³, NR¹⁴SOR¹², NR¹⁴SO₂R¹², NR¹⁴SO₂NR¹²R¹³, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted 3-8 membered cycloalkyl, optionally substituted C₃-C₈ cycloalkoxy, optionally substituted 4-8 membered heterocyclyl, optionally substituted aryl, and optionally substituted heteroaryl; wherein
R¹², R¹³, and R¹⁴ are independently selected from hydrogen, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted 3-8 membered cycloalkyl, optionally substituted C₃-C₈ cycloalkoxy, optionally substituted 4-8 membered heterocyclyl, optionally substituted aryl, and optionally substituted heteroaryl; or
R¹² and R¹³, R¹² and R¹⁴, R¹³ and R¹⁴ together with the atom to which they are connected form a 4-20 membered heterocyclyl ring;
a moiety according to FORMULA 2A: 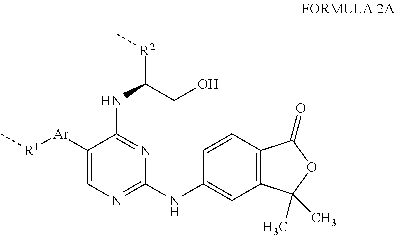
wherein
the “Linker” moiety of the bivalent compound is attached independently to R¹ or R² (indicated by dotted line);
the definitions of R¹, R² and Ar are the same as for FORMULA 2;
a moiety according to FORMULA 2B: 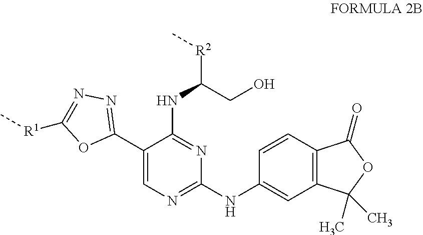
wherein
the “Linker” moiety of the bivalent compound is attached independently to R¹ or R² (indicated by dotted line);
the definitions of R¹ and R² are the same as for FORMULA 2;
a moiety according to FORMULA 2C: 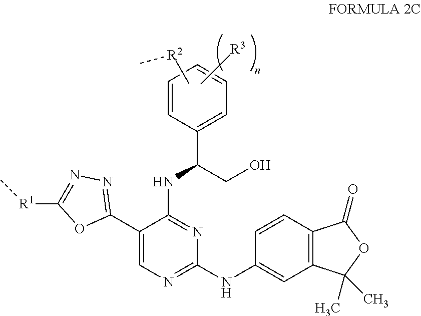
wherein
the “Linker” moiety of the bivalent compound is attached independently to R¹ or R² (indicated by dotted line);
the definitions of R¹ and R² are the same as for FORMULA 2;
R³ is, at each occurrence, independently selected from hydrogen, halogen, oxo, CN, NO₂, OR¹⁵, SR¹⁵, NR¹⁵R¹⁶, C(O)R¹⁶, C(O)OR¹⁵, C(O)NR¹⁵R¹⁶, S(O)R¹, S(O)₂R¹⁵, S(O)₂NR¹⁵R¹⁶, NR¹⁷C(O)OR¹⁵, NR⁶C(O)R¹⁵, NR¹⁷C(O)NR¹⁵R¹⁶, NR¹⁷S(O)R¹⁵, NR¹⁷S(O)₂R¹⁵, NR¹⁷S(O)₂NR¹⁵R¹⁶, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl; wherein
R¹⁵, R¹⁶, and R¹⁷ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl; or
R¹⁵ and R¹⁶, R¹⁵ and R¹⁷, R¹⁶ and R¹⁷ together with the atom to which they are connected form an optionally substituted 3-20 membered cycloalkyl or heterocyclyl ring;
n is independently selected from 0, 1, 2, 3 and 4;
a moiety according to FORMULA 3: 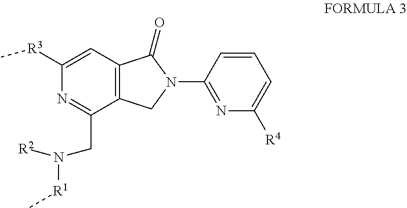
wherein
the “Linker” moiety of the bivalent compound is attached independently to R¹ or R³ (indicated by dotted line);
when the “Linker” moiety of the bivalent compound is attached to R³;
R¹ and R² are, at each occurrence, independently selected from null, hydrogen, C(O)R¹⁰, C(O)OR¹⁰, C(O)NR¹⁰R¹¹, S(O)R¹⁰, S(O)₂R¹⁰, S(O)₂NR¹⁰R¹¹, NR¹²C(O)OR¹⁰, NR¹²C(O)R¹⁰, NR¹²C(O)NR¹⁰R¹¹, NR¹²S(O)R¹⁰, NR¹²S(O)₂R¹⁰, NR¹²S(O)₂NR¹⁰R¹¹, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl; wherein
R¹⁰, R¹¹, and R¹² are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl; or
R¹⁰ and R¹¹, R¹⁰ and R¹², R¹¹ and R¹² together with the atom to which they are connected form an optionally substituted 3-20 membered cycloalkyl or heterocyclyl ring;
R¹ and R² are, at each occurrence, optionally, together with the atom to which they are connected, form an optionally substituted 3-20 membered cycloalkyl or heterocyclyl ring;
R³ is selected from OR¹³, SR¹³, NR¹³R¹⁴, CH₂NR¹³R¹⁴, CH₂CH₂NR¹³R¹⁴, C₁-C₈ alkylene NR¹³R¹⁴, C(O)R¹³, C(O)OR¹³, C(O)NR¹³R¹⁴, S(O)R¹³, S(O)₂R¹⁴, S(O)₂NR¹³R¹⁴, NR¹⁵C(O)OR¹³, NR¹⁵C(O)R¹³, NR¹⁵C(O)NR¹³R¹⁴, NR¹⁵S(O)R³², NR¹⁵S(O)₂R³², NR¹⁵S(O)₂NR¹³R¹⁴ optionally substituted C₁-C₈ alkylene, optionally substituted C₂-C₈ alkenylene, optionally substituted C₂-C₈ alkynylene, optionally substituted C₁-C₈alkoxyC₁-C₈alkylene, optionally substituted C₁-C₈alkylaminoC₁-C₈alkylene, optionally substituted 3-8 membered cycloalkylene, optionally substituted 3-8 membered heterocyclylene, optionally substituted aryl, and optionally substituted heteroaryl, wherein
R¹³ is null, or a bivalent moiety selected from optionally substituted C₁-C₈ alkylenyl, optionally substituted C₂-C₈ alkenylene, optionally substituted C₂-C₈ alkynylene, optionally substituted C₃-C₈ cycloalkylene, optionally substituted 3-8 membered heterocyclylene, optionally substituted arylene, and optionally substituted heteroarylene;
R¹⁴ and R¹⁵ are independently selected from optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₈ cycloalkyl, optionally substituted 3-8 membered heterocyclyl, optionally substituted C₂-C₈ alkenyl, optionally substituted C₂-C₈ alkynyl, optionally substituted aryl, and optionally substituted heteroaryl; or
R¹³ and R¹⁴, R¹³ and R¹⁵, R¹⁴ and R¹⁵ together with the atom to which they are connected form a 3-20 membered cycloalkyl or heterocyclyl ring;
R⁴ is at each occurrence, independently selected from hydrogen, halogen, oxo, CN, NO₂, OR¹⁶, SR¹⁶, NR¹⁶R¹⁷, C(O)R¹⁶, C(O)OR¹⁶, C(O)NR¹⁶R¹⁷, S(O)R¹⁶, S(O)₂R¹⁶, S(O)₂NR¹⁶R¹⁷, NR¹⁸C(O)OR¹⁶, NR¹⁸C(O)R¹⁶, NR¹⁸C(O)NR¹⁶R¹⁷, NR¹⁸S(O)R¹⁶, NR¹⁸S(O)₂R¹⁶, NR¹⁸S(O)₂NR¹⁶R¹⁵, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl; wherein
R¹⁶, R¹⁷, and R¹⁸ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl; or
R¹⁶ and R¹⁷, R¹⁶ and R¹⁸, R¹⁷ and R¹⁸ together with the atom to which they are connected form an optionally substituted 3-20 membered cycloalkyl or heterocyclyl ring;
when the “Linker” moiety of the bivalent compound is attached to R¹;
R¹ is selected from OR¹⁹, SR¹⁹, NR¹⁹R²⁰, CH₂NR¹⁹R²⁰, CH₂CH₂NR¹⁹R²⁰, C₁-C₈ alkylene NR¹⁹R²⁰, C(O)R¹⁹, C(O)OR¹⁹, C(O)NR¹⁹R²⁰, S(O)R¹⁹, S(O)₂R²⁰, S(O)₂NR¹⁹R²⁰, NR²¹C(O)OR¹⁹, NR²¹C(O)R¹⁹, NR²¹C(O)NR¹⁹R²⁰, NR²¹S(O)R¹⁹, NR²¹S(O)₂R¹⁹, NR²¹S(O)₂NR¹⁹R²⁰ optionally substituted C₁-C₈ alkylene, optionally substituted C₂-C₈ alkenylene, optionally substituted C₂-C₈ alkynylene, optionally substituted C₁-C₈alkoxyC₁-C₈alkylene, optionally substituted C₁-C₈alkylaminoC₁-C₈alkylene, optionally substituted 3-8 membered cycloalkylene, optionally substituted 3-8 membered heterocyclylene, optionally substituted aryl, and optionally substituted heteroaryl, wherein
R¹⁹ is null, or a bivalent moiety selected from optionally substituted C₁-C₈ alkylenyl, optionally substituted C₂-C₈ alkenylene, optionally substituted C₂-C₈ alkynylene, optionally substituted C₃-C₈ cycloalkylene, optionally substituted 3-8 membered heterocyclylene, optionally substituted arylene, and optionally substituted heteroarylene;
R²⁰ and R²¹ are independently selected from optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₈ cycloalkyl, optionally substituted 3-8 membered heterocyclyl, optionally substituted C₂-C₈ alkenyl, optionally substituted C₂-C₈ alkynyl, optionally substituted aryl, and optionally substituted heteroaryl; or
R¹⁹ and R²⁰, R¹⁹ and R²¹, R²⁰ and R²¹ together with the atom to which they are connected form a 3-20 membered cycloalkyl or heterocyclyl ring;
R² is, at each occurrence, independently selected from null, hydrogen, C(O)R¹⁰, C(O)OR¹⁰, C(O)NR¹⁰R¹¹, S(O)R¹⁰, S(O)₂R¹⁰, S(O)₂NR¹⁰R¹¹, NR¹²C(O)OR¹⁰, NR¹²C(O)R¹⁰, NR¹²C(O)NR¹⁰R¹¹, NR¹²S(O)R¹⁰, NR¹²S(O)₂R¹°, NR¹²S(O)₂NR¹⁰R¹¹, optionally substituted C₁—C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl; wherein
R¹⁰, R¹¹, and R¹² are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl; or
R¹⁰ and R¹¹, R¹⁰ and R¹², R¹¹ and R¹² together with the atom to which they are connected form an optionally substituted 3-20 membered cycloalkyl or heterocyclyl ring;
R³ and R⁴ are, at each occurrence, independently selected from hydrogen, halogen, oxo, CN, NO₂, OR¹⁶, SR¹⁶, NR¹⁶R¹⁷, C(O)R¹⁶, C(O)OR¹⁶, C(O)NR¹⁶R¹⁷, S(O)R¹⁶, S(O)₂R¹⁶, S(O)₂NR¹⁶R¹⁷, NR¹⁸C(O)OR¹⁶, NR¹⁸C(O)R¹⁶, NR¹⁸C(O)NR¹⁶R¹⁷, NR¹⁸S(O)R¹⁶, NR¹⁸S(O)₂R¹⁶, NR¹⁸S(O)₂NR¹⁶R¹⁵, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl; wherein
R¹⁶, R¹⁷, and R¹⁸ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl; or
R¹⁶ and R¹⁷, R¹⁶ and R¹⁸, R¹⁷ and R¹⁸ together with the atom to which they are connected form an optionally substituted 3-20 membered cycloalkyl or heterocyclyl ring;
a moiety according to FORMULA 3′: 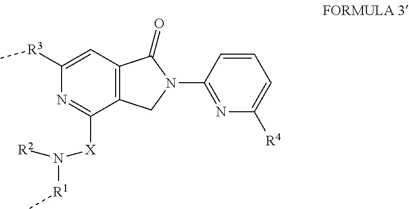
wherein X is absent or when present is CH₂,
the “Linker” moiety of the bivalent compound is attached independently to R¹ or R³ (indicated by dotted line);
when the “Linker” moiety of the bivalent compound is attached to R³;
R¹ and R² are, at each occurrence, independently selected from null, hydrogen, C(O)R¹⁰, C(O)OR¹⁰, C(O)NR¹⁰R¹¹, S(O)R¹⁰, S(O)₂R¹⁰, S(O)₂NR¹⁰R¹¹, NR¹²C(O)OR¹⁰, NR¹²C(O)R¹⁰, NR¹²C(O)NR¹⁰R¹¹, NR¹²S(O)R¹⁰, NR¹²S(O)₂R¹⁰, NR¹²S(O)₂NR¹⁰R¹¹, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl; wherein
R¹⁰, R¹¹, and R¹² are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl; or
R¹⁰ and R¹¹, R¹⁰ and R¹², R¹¹ and R¹² together with the atom to which they are connected form an optionally substituted 3-20 membered cycloalkyl or heterocyclyl ring;
R¹ and R² are, at each occurrence, optionally, together with the atom to which they are connected, form an optionally substituted 3-20 membered cycloalkyl or heterocyclyl ring;
R³ is selected from OR¹³, SR¹³, NR¹³R¹⁴, CH₂NR¹³R¹⁴, CH₂CH₂NR¹³R¹⁴, C₁-C₈ alkylene NR¹³R¹⁴, C(O)R¹³, C(O)OR¹³, C(O)NR¹³R¹⁴, S(O)R¹³, S(O)₂R¹⁴, S(O)₂NR¹³R¹⁴, NR¹⁵C(O)OR¹³, NR¹⁵C(O)R¹³, NR¹⁵C(O)NR¹³R¹⁴, NR¹⁵S(O)R³², NR¹⁵S(O)₂R³², NR¹⁵S(O)₂NR¹³R¹⁴ optionally substituted C₁-C₈ alkylene, optionally substituted C₂-C₈ alkenylene, optionally substituted C₂-C₈ alkynylene, optionally substituted C₁-C₈alkoxyC₁-C₈alkylene, optionally substituted C₁-C₈alkylaminoC₁-C₈alkylene, optionally substituted 3-8 membered cycloalkylene, optionally substituted 3-8 membered heterocyclylene, optionally substituted aryl, and optionally substituted heteroaryl, wherein
R¹³ is null, or a bivalent moiety selected from optionally substituted C₁-C₈ alkylenyl, optionally substituted C₂-C₈ alkenylene, optionally substituted C₂-C₈ alkynylene, optionally substituted C₃-C₈ cycloalkylene, optionally substituted 3-8 membered heterocyclylene, optionally substituted arylene, and optionally substituted heteroarylene;
R¹⁴ and R¹⁵ are independently selected from optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₈ cycloalkyl, optionally substituted 3-8 membered heterocyclyl, optionally substituted C₂-C₈ alkenyl, optionally substituted C₂-C₈ alkynyl, optionally substituted aryl, and optionally substituted heteroaryl; or
R¹³ and R¹⁴, R¹³ and R¹⁵, R¹⁴ and R¹⁵ together with the atom to which they are connected form a 3-20 membered cycloalkyl or heterocyclyl ring;
R⁴ is at each occurrence, independently selected from hydrogen, halogen, oxo, CN, NO₂, OR¹⁶, SR¹⁶, NR¹⁶R¹⁷, C(O)R¹⁶, C(O)OR¹⁶, C(O)NR¹⁶R¹⁷, S(O)R¹⁶, S(O)₂R¹⁶, S(O)₂NR¹⁶R¹⁷, NR¹⁸C(O)OR¹⁶, NR¹⁸C(O)R¹⁶, NR¹⁸C(O)NR¹⁶R¹⁷, NR¹⁸S(O)R¹⁶, NR¹⁸S(O)₂R¹⁶, NR¹⁸S(O)₂NR¹⁶R¹⁵, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl; wherein
R¹⁶, R¹⁷, and R¹⁸ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl; or
R¹⁶ and R¹⁷, R¹⁶ and R¹⁸, R¹⁷ and R¹⁸ together with the atom to which they are connected form an optionally substituted 3-20 membered cycloalkyl or heterocyclyl ring;
when the “Linker” moiety of the bivalent compound is attached to R¹;
R¹ is selected from OR¹⁹, SR¹⁹, NR¹⁹R²⁰, CH₂NR¹⁹R²⁰, CH₂CH₂NR¹⁹R²⁰, C₁-C₈ alkylene NR¹⁹R²⁰, C(O)R¹⁹, C(O)OR¹⁹, C(O)NR¹⁹R²⁰, S(O)R¹⁹, S(O)₂R²⁰, S(O)₂NR¹⁹R²⁰, NR²¹C(O)OR¹⁹, NR²¹C(O)R¹⁹, NR²¹C(O)NR¹⁹R²⁰, NR²¹S(O)R¹⁹, NR²¹S(O)₂R¹⁹, NR²¹S(O)₂NR¹⁹R²⁰ optionally substituted C₁-C₈ alkylene, optionally substituted C₂-C₈ alkenylene, optionally substituted C₂-C₈ alkynylene, optionally substituted C₁-C₈alkoxyC₁-C₈alkylene, optionally substituted C₁-C₈alkylaminoC₁-C₈alkylene, optionally substituted 3-8 membered cycloalkylene, optionally substituted 3-8 membered heterocyclylene, optionally substituted aryl, and optionally substituted heteroaryl, wherein
R¹⁹ is null, or a bivalent moiety selected from optionally substituted C₁-C₈ alkylenyl, optionally substituted C₂-C₈ alkenylene, optionally substituted C₂-C₈ alkynylene, optionally substituted C₃-C₈ cycloalkylene, optionally substituted 3-8 membered heterocyclylene, optionally substituted arylene, and optionally substituted heteroarylene;
R²⁰ and R²¹ are independently selected from optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₈ cycloalkyl, optionally substituted 3-8 membered heterocyclyl, optionally substituted C₂-C₈ alkenyl, optionally substituted C₂-C₈ alkynyl, optionally substituted aryl, and optionally substituted heteroaryl; or
R¹⁹ and R²⁰, R¹⁹ and R²¹, R²⁰ and R²¹ together with the atom to which they are connected form a 3-20 membered cycloalkyl or heterocyclyl ring;
R² is, at each occurrence, independently selected from null, hydrogen, C(O)R¹⁰, C(O)OR¹⁰, C(O)NR¹⁰R¹¹, S(O)R¹⁰, S(O)₂R¹⁰, S(O)₂NR¹⁰R¹¹, NR¹²C(O)OR¹⁰, NR¹²C(O)R¹⁰, NR¹²C(O)NR¹⁰R¹¹, NR¹²S(O)R¹⁰, NR¹²S(O)₂R¹°, NR¹²S(O)₂NR¹⁰R¹¹, optionally substituted C₁—C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl; wherein
R¹⁰, R¹¹, and R¹² are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl; or
R¹⁰ and R¹¹, R¹⁰ and R¹², R¹¹ and R¹² together with the atom to which they are connected form an optionally substituted 3-20 membered cycloalkyl or heterocyclyl ring;
R³ and R⁴ are, at each occurrence, independently selected from hydrogen, halogen, oxo, CN, NO₂, OR¹⁶, SR¹⁶, NR¹⁶R¹⁷, C(O)R¹⁶, C(O)OR¹⁶, C(O)NR¹⁶R¹⁷, S(O)R¹⁶, S(O)₂R¹⁶, S(O)₂NR¹⁶R¹⁷, NR¹⁸C(O)OR¹⁶, NR¹⁸C(O)R¹⁶, NR¹⁸C(O)NR¹⁶R¹⁷, NR¹⁸S(O)R¹⁶, NR¹⁸S(O)₂R¹⁶, NR¹⁸S(O)₂NR¹⁶R¹⁵, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl; wherein
R¹⁶, R¹⁷, and R¹⁸ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl; or
R¹⁶ and R¹⁷, R¹⁶ and R¹⁸, R¹⁷ and R¹⁸ together with the atom to which they are connected form an optionally substituted 3-20 membered cycloalkyl or heterocyclyl ring;
a moiety according to FORMULA 3A: 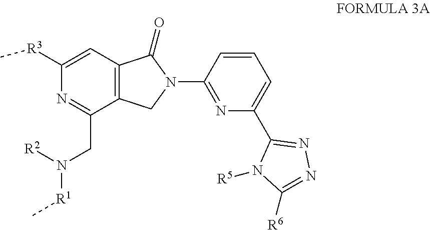
wherein
the “Linker” moiety of the bivalent compound is attached independently to R¹ or R³ (indicated by dotted line);
the definitions of, R¹, R² and R³ are the same as for FORMULA 3;
R⁵ is, at each occurrence, independently selected from null, hydrogen, C(O)R²², C(O)OR²², C(O)NR²²R²³, S(O)R²², S(O)₂R²², S(O)₂NR²²R²³, NR²⁴C(O)OR²², NR²⁴C(O)R²², NR²⁴C(O)NR²²R²³, NR²⁴S(O)R²², NR²⁴S(O)₂R²², NR²⁴S(O)₂NR²²R²³, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl; wherein
R²², R²³, and R²⁴ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl; or
R²² and R²³, R²² and R²⁴, R²³ and R²⁴ together with the atom to which they are connected form an optionally substituted 3-20 membered cycloalkyl or heterocyclyl ring;
R⁶ is, at each occurrence, independently selected from hydrogen, halogen, oxo, CN, NO₂, OR²⁵, SR²⁵, NR²⁵R²⁶, C(O)R²⁵, C(O)OR²⁵, C(O)NR²⁵R²⁶, S(O)R²⁵, S(O)₂R²⁵, S(O)₂NR²⁵R²⁶, NR²⁷C(O)OR²⁵, NR²⁷C(O)R²⁵, NR²¹C(O)NR²⁵R²⁶, NR²⁷S(O)R²⁵, NR²⁷S(O)₂R²⁵, NR²⁷S(O)₂NR²⁵R²⁶, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl; wherein
R²⁵, R²⁶, and R²⁷ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl; or
R²⁵ and R²⁶, R²⁵ and R²⁶, R²⁶ and R²⁷ together with the atom to which they are connected form an optionally substituted 3-20 membered cycloalkyl or heterocyclyl ring;
a moiety according to FORMULA 3A′: 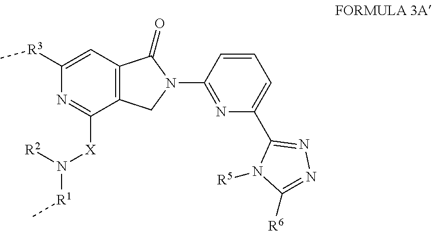
wherein X is absent or when present is CH₂,
the “Linker” moiety of the bivalent compound is attached independently to R¹ or R³ (indicated by dotted line);
the definitions of, R¹, R² and R³ are the same as for FORMULA 3;
R⁵ is, at each occurrence, independently selected from null, hydrogen, C(O)R²², C(O)OR²², C(O)NR²²R²³, S(O)R²², S(O)₂R²², S(O)₂NR²²R²³, NR²⁴C(O)OR²², N²⁴C(O)R²², NR²⁴C(O)NR²²R²³, NR²⁴S(O)R²², NR²⁴S(O)₂R²², NR²⁴S(O)₂NR²²R²³, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl; wherein
R²², R²³, and R²⁴ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl; or
R²² and R²³, R²² and R²⁴, R²³ and R²⁴ together with the atom to which they are connected form an optionally substituted 3-20 membered cycloalkyl or heterocyclyl ring;
R⁶ is, at each occurrence, independently selected from hydrogen, halogen, oxo, CN, NO₂, OR²⁵, SR²⁵, NR²⁵R²⁶, C(O)R²⁵, C(O)OR²⁵, C(O)NR²⁵R²⁶, S(O)R²⁵, S(O)₂R²⁵, S(O)₂NR²⁵R²⁶, NR²⁷C(O)OR²⁵, NR²⁷C(O)R²⁵, NR²¹C(O)NR²⁵R²⁶, NR²⁷S(O)R²⁵, NR²⁷S(O)₂R²⁵, NR²⁷S(O)₂NR²⁵R²⁶, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl; wherein
R²⁵, R²⁶, and R²⁷ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl; or
R²⁵ and R²⁶, R²⁵ and R²⁶, R²⁶ and R²⁷ together with the atom to which they are connected form an optionally substituted 3-20 membered cycloalkyl or heterocyclyl ring;
a moiety according to FORMULA 3B: 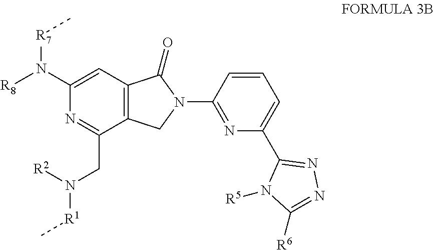
wherein
the “Linker” moiety of the bivalent compound is attached independently to R¹ or R⁷ (indicated by dotted line);
the definitions of R⁵ and R⁶ are the same as for FORMULA 3A;
when the “Linker” moiety of the bivalent compound is attached to R¹;
R¹ is selected from OR²⁸, SR²⁸, NR²⁸R²⁹, CH₂NR²⁸R²⁹, CH₂CH₂NR²⁸R²⁹, C₁-C₈ alkylene NR²⁸R²⁹, C(O)R²⁸, C(O)OR²⁸, C(O)NR²⁸R²⁹, S(O)R²⁸, S(O)₂R²⁹, S(O)₂NR²⁸R²⁹, NR³⁰C(O)OR²⁸, NR³⁰C(O)R²⁸, NR³⁰C(O)NR²⁸R²⁹, NR³⁰S(O)R²⁸, NR³⁰S(O)₂R²⁸, NR³⁰S(O)₂NR²⁸R²⁹ optionally substituted C₁-C₈ alkylene, optionally substituted C₂-C₈ alkenylene, optionally substituted C₂-C₈ alkynylene, optionally substituted C₁-C₈alkoxyC₁-C₈alkylene, optionally substituted C₁-C₈alkylaminoC₁-C₈alkylene, optionally substituted 3-8 membered cycloalkylene, optionally substituted 3-8 membered heterocyclylene, optionally substituted aryl, and optionally substituted heteroaryl, wherein
R²⁸ is null, or a bivalent moiety selected from optionally substituted C₁-C₈ alkylenyl, optionally substituted C₂-C₈ alkenylene, optionally substituted C₂-C₈ alkynylene, optionally substituted C₃-C₈ cycloalkylene, optionally substituted 3-8 membered heterocyclylene, optionally substituted arylene, and optionally substituted heteroarylene;
R²⁹ and R³⁰ are independently selected from optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₈ cycloalkyl, optionally substituted 3-8 membered heterocyclyl, optionally substituted C₂-C₈ alkenyl, optionally substituted C₂-C₈ alkynyl, optionally substituted aryl, and optionally substituted heteroaryl; or
R²⁸ and R²⁹, R²⁸ and R²⁹, R²⁹ and R³⁰ together with the atom to which they are connected form a 3-20 membered cycloalkyl or heterocyclyl ring;
R², R⁷ and R⁸ are, at each occurrence, independently selected from null, hydrogen, C(O)R³¹, C(O)OR³¹, C(O)NR³¹R³², S(O)R³¹, S(O)₂R³¹, S(O)₂NR³¹R³², NR³³C(O)OR³¹, NR³³C(O)R³¹, NR³³C(O)NR³¹R³², NR³³S(O)R³¹, NR³³S(O)₂R³¹, NR³³S(O)₂NR³¹R³², optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl; wherein
R³¹, R³², and R³³ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl; or
R³¹ and R³², R³¹ and R³³, R³² and R³³ together with the atom to which they are connected form an optionally substituted 3-20 membered cycloalkyl or heterocyclyl ring;
R⁷ and R⁸ are, at each occurrence, optionally, together with the atom to which they are connected, form an optionally substituted 3-20 membered cycloalkyl or heterocyclyl ring;
when the “Linker” moiety of the bivalent compound is attached to R⁷;
R⁷ is selected from OR²⁸, SR²⁸, NR²⁸R²⁹, CH₂NR²⁸R²⁹, CH₂CH₂NR²⁸R²⁹, C₁-C₈ alkylene NR²⁸R²⁹, C(O)R²⁸, C(O)OR²⁸, C(O)NR²⁸R²⁹, S(O)R²⁸, S(O)₂R²⁹, S(O)₂NR²⁸R²⁹, NR³⁰C(O)OR²⁸, NR³⁰C(O)R²⁸, NR³⁰C(O)NR²⁸R²⁹, NR³⁰S(O)R²⁸, NR³⁰S(O)₂R²⁸, NR³⁰S(O)₂NR²⁸R²⁹ optionally substituted C₁-C₈ alkylene, optionally substituted C₂-C₈ alkenylene, optionally substituted C₂-C₈ alkynylene, optionally substituted C₁-C₈alkoxyC₁-C₈alkylene, optionally substituted C₁-C₈alkylaminoC₁-C₈alkylene, optionally substituted 3-8 membered cycloalkylene, optionally substituted 3-8 membered heterocyclylene, optionally substituted aryl, and optionally substituted heteroaryl, wherein
R²⁸ is null, or a bivalent moiety selected from optionally substituted C₁-C₈ alkylenyl, optionally substituted C₂-C₈ alkenylene, optionally substituted C₂-C₈ alkynylene, optionally substituted C₃-C₈ cycloalkylene, optionally substituted 3-8 membered heterocyclylene, optionally substituted arylene, and optionally substituted heteroarylene;
R²⁹ and R³⁰ are independently selected from optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₈ cycloalkyl, optionally substituted 3-8 membered heterocyclyl, optionally substituted C₂-C₈ alkenyl, optionally substituted C₂-C₈ alkynyl, optionally substituted aryl, and optionally substituted heteroaryl; or
R²⁸ and R²⁹, R²⁸ and R²⁹, R²⁹ and R³⁰ together with the atom to which they are connected form a 3-20 membered cycloalkyl or heterocyclyl ring;
R¹, R² and R⁸ are, at each occurrence, independently selected from null, hydrogen, C(O)R³¹, C(O)OR³¹, C(O)NR³¹R³², S(O)R³¹, S(O)₂R³¹, S(O)₂NR³¹R³², NR³³C(O)OR³¹, NR³³C(O)R³¹, NR³³C(O)NR³¹R³², NR³³S(O)R³¹, NR³³S(O)₂R³¹, NR³³S(O)₂NR³¹R³², optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl; wherein
R³¹, R³², and R³³ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl; or
R³¹ and R³², R³¹ and R³³, R³² and R³³ together with the atom to which they are connected form an optionally substituted 3-20 membered cycloalkyl or heterocyclyl ring;
R¹ and R² are, at each occurrence, optionally, together with the atom to which they are connected, form an optionally substituted 3-20 membered cycloalkyl or heterocyclyl ring;
a moiety according to FORMULA 4: 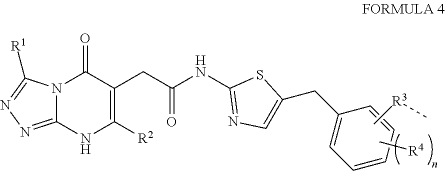
wherein
the “Linker” moiety of the bivalent compound is attached independently to R³ (indicated by dotted line);
R^{1′}R² and R⁴ are, at each occurrence, independently selected from hydrogen, halogen, oxo, CN, NO₂, OR⁸, SR⁵, NR⁵R⁶, C(O)R⁵, C(O)OR⁵, C(O)NR⁵R⁶, S(O)R⁵, S(O)₂R⁵, S(O)₂NR⁵R⁶, NR⁷C(O)OR⁵, NR⁷C(O)R⁵, NR⁷C(O)NR²⁵R²⁶, NR⁷S(O)R⁵, NR⁷S(O)₂R⁵, NR⁷S(O)₂NR⁵R⁶, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl; wherein
R⁵, R⁶, and R⁷ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl; or
R⁵ and R⁶, R⁵ and R⁷, R⁶ and R⁷ together with the atom to which they are connected form an optionally substituted 3-20 membered cycloalkyl or heterocyclyl ring;
n is independently selected from 0, 1, 2, 3 and 4;
R³ is selected from OR⁸, SR⁵, NR⁸R⁹, CH₂NR⁸R⁹, CH₂CH₂NR⁸R⁹, C₁-C₈ alkylene NR⁸R⁹, C(O)R⁸, C(O)OR⁹, C(O)NR⁸R⁹, S(O)R⁸, S(O)₂R⁹, S(O)₂NR⁸R⁹, NR¹⁰C(O)OR⁹, NR¹⁰C(O)R⁹, NR¹⁰C(O)NR⁸R⁹, NR¹⁰S(O)R⁸, NR¹⁰S(O)₂R⁸, NR¹⁰S(O)₂NR⁸R⁹ optionally substituted C₁-C₈ alkylene, optionally substituted C₂-C₈ alkenylene, optionally substituted C₂-C₈ alkynylene, optionally substituted C₁-C₈alkoxyC₁-C₈alkylene, optionally substituted C₁-C₈alkylaminoC₁-C₈alkylene, optionally substituted 3-8 membered cycloalkylene, optionally substituted 3-8 membered heterocyclylene, optionally substituted aryl, and optionally substituted heteroaryl, wherein
R⁸ is null, or a bivalent moiety selected from optionally substituted C₁-C₈ alkylenyl, optionally substituted C₂-C₈ alkenylene, optionally substituted C₂-C₈ alkynylene, optionally substituted C₃-C₈ cycloalkylene, optionally substituted 3-8 membered heterocyclylene, optionally substituted arylene, and optionally substituted heteroarylene;
R⁹ and R¹⁰ are independently selected from optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₈ cycloalkyl, optionally substituted 3-8 membered heterocyclyl, optionally substituted C₂-C₈ alkenyl, optionally substituted C₂-C₈ alkynyl, optionally substituted aryl, and optionally substituted heteroaryl; or
R⁸ and R⁹, R⁸ and R¹⁰, R⁹ and R¹⁰ together with the atom to which they are connected form a 3-20 membered cycloalkyl or heterocyclyl ring;
a moiety according to FORMULA 4A: 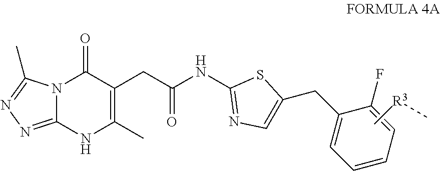
wherein
the “Linker” moiety of the bivalent compound is attached independently to R³ (indicated by dotted line);
the definition of R³ is the same as for FORMULA 4; and
pharmaceutically acceptable salts thereof.

2. The bivalent compound of claim 1, wherein the HPK1 ligand is the moiety according to FORMULA 1.

3. The bivalent compound of claim 1, wherein the HPK1 ligand is the moiety according to FORMULA 2.

4. The bivalent compound of claim 1, wherein the HPK1 ligand is the moiety according to FORMULA 2A.

5. The bivalent compound of claim 1, wherein the HPK1 ligand is the moiety according to FORMULA 2B.

6. The bivalent compound of claim 1, wherein the HPK1 ligand is the moiety according to FORMULA 2C.

7. The bivalent compound of claim 1, wherein the HPK1 ligand is the moiety according to FORMULA 3.

8. The bivalent compound of claim 1, wherein the HPK1 ligand is the moiety according to FORMULA 3A.

9. The bivalent compound of claim 1, wherein the HPK1 ligand is the moiety according to FORMULA 3B.

10. The bivalent compound of claim 1, wherein the HPK1 ligand is the moiety according to FORMULA 4.

11. The bivalent compound of claim 1, wherein the HPK1 ligand is the moiety according to FORMULA 4A.

12. The bivalent compound of claim 1, wherein the degradation/disruption tag is selected from the group consisting of: a moiety according to one of FORMULAE 12A, 12B, 12C and 12D: 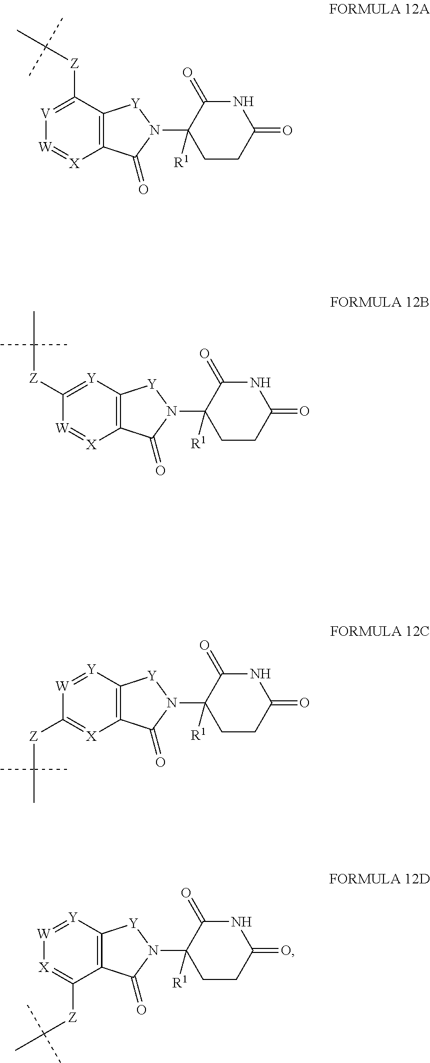
wherein
V, W, and X are independently selected from CR² and N;
Y is selected from CO, CR³R⁴, and N═N;
Z is selected from null, CO, CR⁵R⁶, NR⁵, O, optionally substituted C₁-C₁₀ alkylene, optionally substituted C₁-C₁₀ alkenylene, optionally substituted C₁-C₁₀ alkynylene, optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl; preferably, Z is selected from null, CH₂, CH═CH, C≡C, NH and O;
R¹, and R² are independently selected from hydrogen, halogen, cyano, nitro, optionally substituted C₁-C₆ alkyl, optionally substituted 3 to 6 membered carbocyclyl, and optionally substituted 4 to 6 membered heterocyclyl;
R³, and R⁴ are independently selected from hydrogen, halogen, cyano, nitro, optionally substituted C₁-C₆ alkyl, optionally substituted 3 to 6 membered carbocyclyl, and optionally substituted 4 to 6 membered heterocyclyl; or R³ and R⁴ together with the atom to which they are connected form a 3-6 membered carbocyclyl, or 4-6 membered heterocyclyl; and
R⁵ and R⁶ are independently selected from null, hydrogen, halogen, oxo, hydroxyl, amino, cyano, nitro, optionally substituted C₁-C₆ alkyl, optionally substituted 3 to 6 membered carbocyclyl, and optionally substituted 4 to 6 membered heterocyclyl; or R⁵ and R⁶ together with the atom to which they are connected form a 3-6 membered carbocyclyl, or 4-6 membered heterocyclyl;
a moiety according to one of FORMULAE 12E, 12F, 12G, 12H, and 12I: 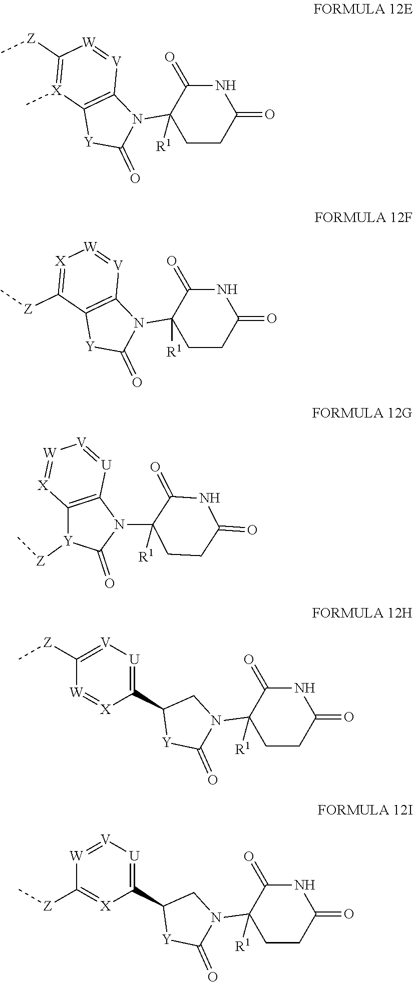
wherein
U, V, W, and X are independently selected from CR² and N;
Y is selected from CR³R⁴, NR³ and O; preferably, Y is selected from CH₂, NH, NCH₃ and O;
Z is selected from null, CO, CR⁵R⁶, NR⁵, O, optionally substituted C₁-C₁₀ alkylene, optionally substituted C₁-C₁₀ alkenylene, optionally substituted C₁-C₁₀ alkynylene, optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl; preferably, Z is selected from null, CH₂, CH═CH, C≡C, NH and O;
R¹, and R² are independently selected from hydrogen, halogen, cyano, nitro, optionally substituted C₁-C₆ alkyl, optionally substituted 3 to 6 membered carbocyclyl, and optionally substituted 4 to 6 membered heterocyclyl;
R³, and R⁴ are independently selected from hydrogen, halogen, cyano, nitro, optionally substituted C₁-C₆ alkyl, optionally substituted 3 to 6 membered carbocyclyl, and optionally substituted 4 to 6 membered heterocyclyl; or R³ and R⁴ together with the atom to which they are connected form a 3-6 membered carbocyclyl, or 4-6 membered heterocyclyl; and
R⁵ and R⁶ are independently selected from null, hydrogen, halogen, oxo, hydroxyl, amino, cyano, nitro, optionally substituted C₁-C₆ alkyl, optionally substituted 3 to 6 membered carbocyclyl, and optionally substituted 4 to 6 membered heterocyclyl; or R⁵ and R⁶ together with the atom to which they are connected form a 3-6 membered carbocyclyl, or 4-6 membered heterocyclyl;
a moiety according to FORMULA 13A: 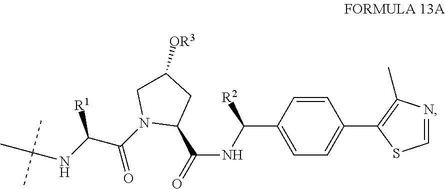
wherein
R¹ and R² are independently selected from hydrogen, optionally substituted C₁-C₈ alkyl, optionally substituted C₁-C₈alkoxyC₁-C₈alkyl, optionally substituted C₁-C₈ haloalkyl, optionally substituted C₁-C₈ hydroxyalkyl, optionally substituted C₁-C₈ aminoalkyl, optionally substituted C₁-C₈alkylaminoC₁-C₈alkyl, optionally substituted C₃-C₇ cycloalkyl, optionally substituted 3-7 membered heterocyclyl, optionally substituted C₂-C₈ alkenyl, and optionally substituted C₂-C₈ alkynyl; and
R³ is hydrogen, optionally substituted C(O)C₁-C₈ alkyl, optionally substituted C(O)C₁-C₈alkoxyC₁-C₈alkyl, optionally substituted C(O)C₁-C₈ haloalkyl, optionally substituted C(O)C₁-C₈ hydroxyalkyl, optionally substituted C(O)C₁-C₈ aminoalkyl, optionally substituted C(O)C₁-C₈alkylaminoC₁-C₈alkyl, optionally substituted C(O)C₃-C₇ cycloalkyl, optionally substituted C(O)(3-7 membered heterocyclyl), optionally substituted C(O)C₂-C₈ alkenyl, optionally substituted C(O)C₂-C₈ alkynyl, optionally substituted C(O)OC₁-C₈alkoxyC₁-C₈alkyl, optionally substituted C(O)OC₁-C₈ haloalkyl, optionally substituted C(O)OC₁-C₈ hydroxyalkyl, optionally substituted C(O)OC₁-C₈ aminoalkyl, optionally substituted C(O)OC₁-C₈alkylaminoC₁-C₈alkyl, optionally substituted C(O)OC₃-C₇ cycloalkyl, optionally substituted C(O)O(3-7 membered heterocyclyl), optionally substituted C(O)OC₂-C₈ alkenyl, optionally substituted C(O)OC₂-C₈ alkynyl, optionally substituted C(O)NC₁-C₈alkoxyC₁-C₈alkyl, optionally substituted C(O)NC₁-C₈ haloalkyl, optionally substituted C(O)NC₁-C₈ hydroxyalkyl, optionally substituted C(O)NC₁-C₈ aminoalkyl, optionally substituted C(O)NC₁-C₈alkylaminoC₁-C₈alkyl, optionally substituted C(O)NC₃-C₇ cycloalkyl, optionally substituted C(O)N(3-7 membered heterocyclyl), optionally substituted C(O)NC₂-C₈ alkenyl, optionally substituted C(O)NC₂-C₈ alkynyl, optionally substituted P(O)(OH)₂, optionally substituted P(O)(OC₁-C₈ alkyl)₂, and optionally substituted P(O)(OC₁-C₈ aryl)₂;
a moiety according to one of FORMULAE 13B, 13C, 13D, 13E and 13F. 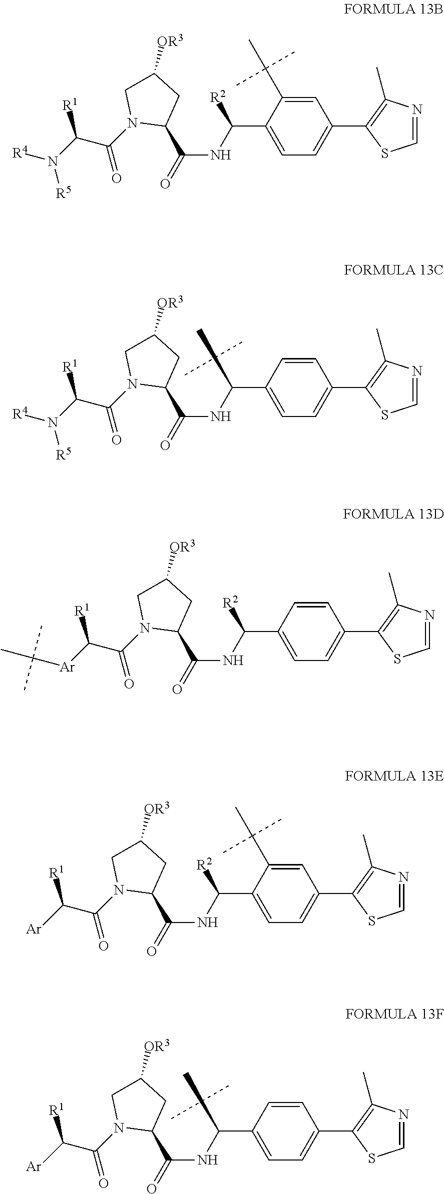
wherein
R¹ and R² are independently selected from hydrogen, halogen, OH, NH₂, CN, optionally substituted C₁-C₈ alkyl, optionally substituted C₁-C₈alkoxyC₁-C₈alkyl, optionally substituted C₁-C₈ haloalkyl, optionally substituted C₁-C₈ hydroxyalkyl, optionally substituted C₁-C₈ aminoalkyl, optionally substituted C₁-C₈alkylaminoC₁-C₈alkyl, optionally substituted C₃-C₇ cycloalkyl, optionally substituted 3-7 membered heterocyclyl, optionally substituted C₂-C₈ alkenyl, and optionally substituted C₂-C₈ alkynyl; (preferably, R¹ is selected from iso-propyl or tert-butyl; and R² is selected from hydrogen or methyl);
R³ is hydrogen, optionally substituted C(O)C₁-C₈ alkyl, optionally substituted C(O)C₁-C₈alkoxyC₁-C₈alkyl, optionally substituted C(O)C₁-C₈ haloalkyl, optionally substituted C(O)C₁-C₈ hydroxyalkyl, optionally substituted C(O)C₁-C₈ aminoalkyl, optionally substituted C(O)C₁-C₈alkylaminoC₁-C₈alkyl, optionally substituted C(O)C₃-C₇ cycloalkyl, optionally substituted C(O)(3-7 membered heterocyclyl), optionally substituted C(O)C₂-C₈ alkenyl, optionally substituted C(O)C₂-C₈ alkynyl, optionally substituted C(O)OC₁-C₈alkoxyC₁-C₈alkyl, optionally substituted C(O)OC₁-C₈ haloalkyl, optionally substituted C(O)OC₁-C₈ hydroxyalkyl, optionally substituted C(O)OC₁-C₈ aminoalkyl, optionally substituted C(O)OC₁-C₈alkylaminoC₁-C₈alkyl, optionally substituted C(O)OC₃-C₇ cycloalkyl, optionally substituted C(O)O(3-7 membered heterocyclyl), optionally substituted C(O)OC₂-C₈ alkenyl, optionally substituted C(O)OC₂-C₈ alkynyl, optionally substituted C(O)NC₁-C₈alkoxyC₁-C₈alkyl, optionally substituted C(O)NC₁-C₈ haloalkyl, optionally substituted C(O)NC₁-C₈ hydroxyalkyl, optionally substituted C(O)NC₁-C₈ aminoalkyl, optionally substituted C(O)NC₁-C₈alkylaminoC₁-C₈alkyl, optionally substituted C(O)NC₃-C₇ cycloalkyl, optionally substituted C(O)N(3-7 membered heterocyclyl), optionally substituted C(O)NC₂-C₈ alkenyl, optionally substituted C(O)NC₂-C₈ alkynyl, optionally substituted P(O)(OH)₂, optionally substituted P(O)(OC₁-C₈ alkyl)₂, and optionally substituted P(O)(OC₁-C₈ aryl)₂; and
R⁴ and R⁵ are independently selected from hydrogen, COR⁶, CO₂R⁶, CONR⁶R⁷, SOR⁶, SO₂R⁶, SO₂NR⁶R⁷, optionally substituted C₁-C₈ alkyl, optionally substituted C₁-C₈alkoxyC₁-C₈alkyl, optionally substituted C₁-C₈alkylaminoC₁-C₈alkyl, optionally substituted 3-8 membered cycloalkyl, optionally substituted 3-8 membered heterocyclyl, optionally substituted aryl, and optionally substituted heteroaryl; wherein
R⁶ and R⁷ are independently selected from hydrogen, optionally substituted C₁-C₈ alkyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted 3-8 membered cycloalkyl, optionally substituted 3-8 membered heterocyclyl, optionally substituted aryl, and optionally substituted heteroaryl; or
R⁴ and R⁵; R⁶ and R⁷ together with the atom to which they are connected form a 4-8 membered cycloalkyl or heterocyclyl ring;
Ar is selected from aryl and heteroaryl, each of which is optionally substituted with one or more substituents independently selected from F, Cl, CN, NO₂, OR⁸, NR⁸R⁹, COR⁸, CO₂R⁸, CONR⁸R⁹, SOR⁸, SO₂R⁸, SO₂NR⁹R¹⁰, NR⁹COR¹⁰, NR⁸C(O)NR⁹R¹⁰, NR⁹SOR¹⁰, NR⁹SO₂R¹⁰, optionally substituted C₁-C₆ alkyl, optionally substituted C₁-C₆ alkoxyalkyl, optionally substituted C₁-C₆ haloalkyl, optionally substituted C₁-C₆ hydroxyalkyl, optionally substituted C₁-C₆alkylaminoC₁-C₆alkyl, optionally substituted C₃-C₇ cycloalkyl, optionally substituted 3-7 membered heterocyclyl, optionally substituted C₂-C₆ alkenyl, optionally substituted C₂-C₆ alkynyl, optionally substituted aryl, and optionally substituted C₄-C₈ heteroaryl; wherein
R⁸, R⁹, and R¹⁰ are independently selected from null, hydrogen, optionally substituted C₁-C₆ alkyl, optionally substituted C₂-C₆ alkenyl, optionally substituted C₂-C₆ alkynyl, optionally substituted C₃-C₇ cycloalkyl, optionally substituted 3-7 membered heterocyclyl, optionally substituted aryl, and optionally substituted heteroaryl; or
R⁸ and R⁹; R⁹ and R¹⁰ together with the atom to which they are connected form a 4-8 membered cycloalkyl or heterocyclyl ring;
a moiety according to FORMULA 14A: 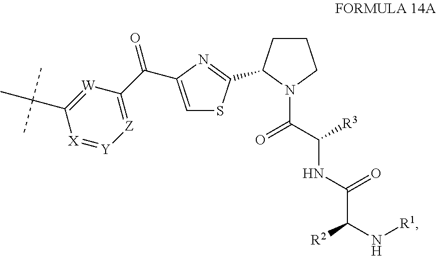
wherein
V, W, X, and Z are independently selected from CR⁴ and N;
R¹, R², R³, and R⁴ are independently selected from hydrogen, optionally substituted C₁-C₈ alkyl, optionally substituted C₁-C₈alkoxyC₁-C₈alkyl, optionally substituted C₁-C₈ haloalkyl, optionally substituted C₁-C₈ hydroxyalkyl, optionally substituted C₃-C₇ cycloalkyl, optionally substituted 3-7 membered heterocyclyl, optionally substituted C₂-C₈ alkenyl, and optionally substituted C₂-C₈ alkynyl; and
a moiety according to FORMULA 14B: 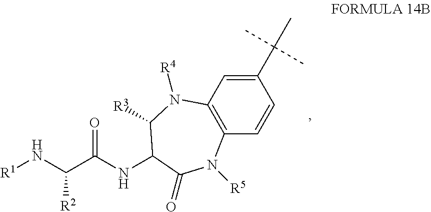
wherein
R¹, R², and R³ are independently selected from hydrogen, halogen, optionally substituted C₁-C₈ alkyl, optionally substituted C₁-C₈alkoxyC₁-C₈alkyl, optionally substituted C₁-C₈ haloalkyl, optionally substituted C₁-C₈ hydroxyalkyl, optionally substituted C₃-C₇ cycloalkyl, optionally substituted 3-7 membered heterocyclyl, optionally substituted C₂-C₈ alkenyl, and optionally substituted C₂-C₈ alkynyl;
R⁴ and R⁵ are independently selected from hydrogen, COR⁶, CO₂R⁶, CONR⁶R⁷, SOR⁶, SO₂R⁶, SO₂NR⁶R⁷, optionally substituted C₁-C₈ alkyl, optionally substituted C₁-C₈alkoxyC₁-C₈alkyl, optionally substituted C₁-C₈alkylaminoC₁-C₈alkyl, optionally substituted aryl-C₁-C₈alkyl, optionally substituted 3-8 membered cycloalkyl, optionally substituted 3-8 membered heterocyclyl, optionally substituted aryl, and optionally substituted heteroaryl; wherein
R⁶ and R⁷ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted 3-8 membered cycloalkyl, optionally substituted 3-8 membered heterocyclyl, optionally substituted aryl, and optionally substituted heteroaryl; or
R⁶ and R⁷ together with the atom to which they are connected form a 4-8 membered cycloalkyl or heterocyclyl ring.

13. The bivalent compound of claim 12, wherein the degradation/disruption tag is the moiety of one of FORMULAE 12A, 12B, 12C and 12D.

14. The bivalent compound of claim 12, wherein the degradation/disruption tag is the moiety of one of FORMULAE 12E, 12F, 12G, 12H and 121.

15. The bivalent compound of claim 12, wherein the degradation/disruption tag is the moiety of FORMULA 13A.

16. The bivalent compound of claim 12, wherein the degradation/disruption tag is the moiety of one of FORMULAE 13B, 13C, 13D, 13E and 13F.

17. The bivalent compound of claim 12, wherein the degradation/disruption tag is the moiety of FORMULA 14A.

18. The bivalent compound of claim 12, wherein the degradation/disruption tag is the moiety of FORMULA 14B.

19. The bivalent compound of claim 1, wherein the degradation/disruption tag is selected from the group consisting of:

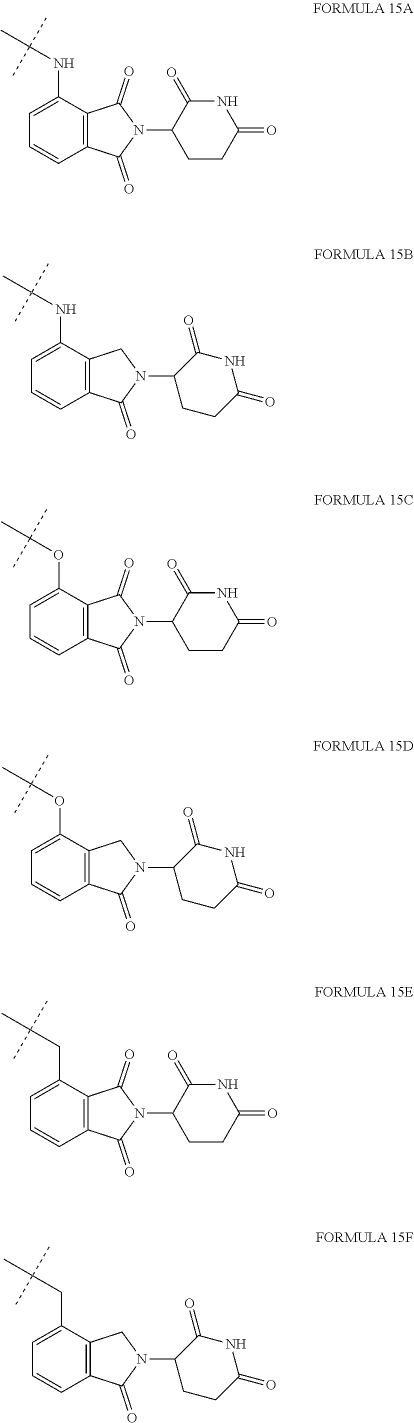

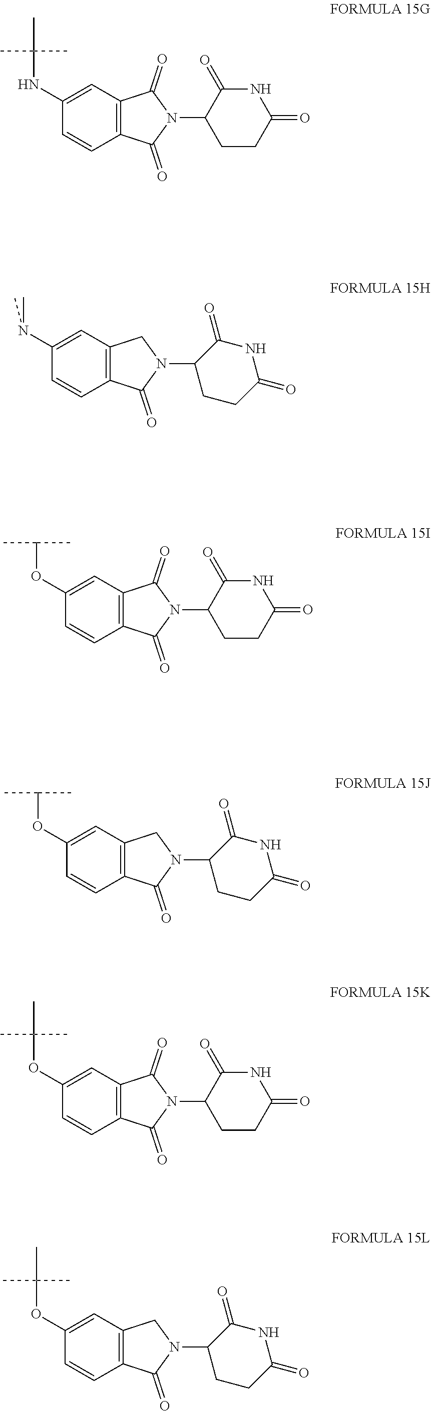

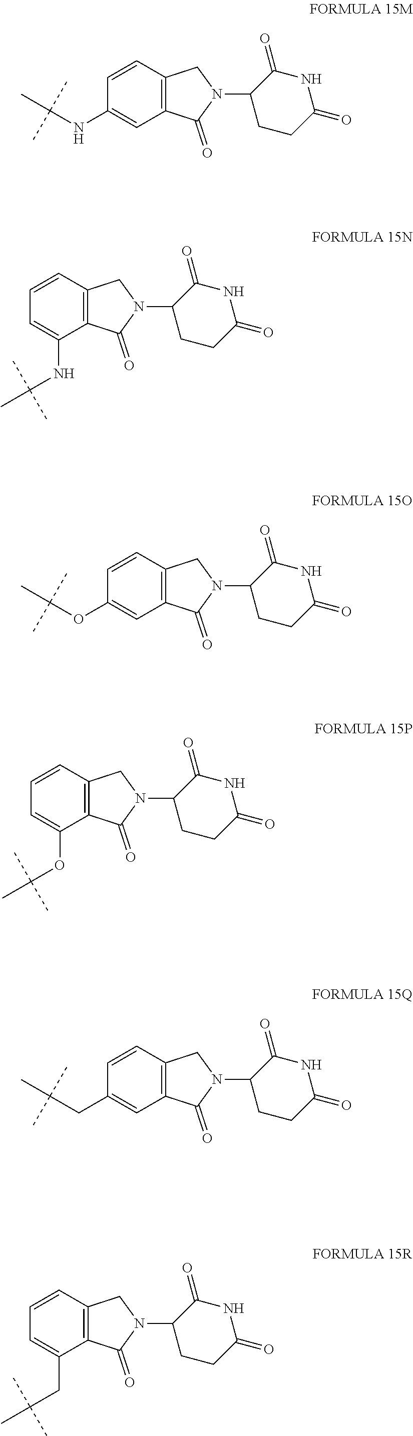

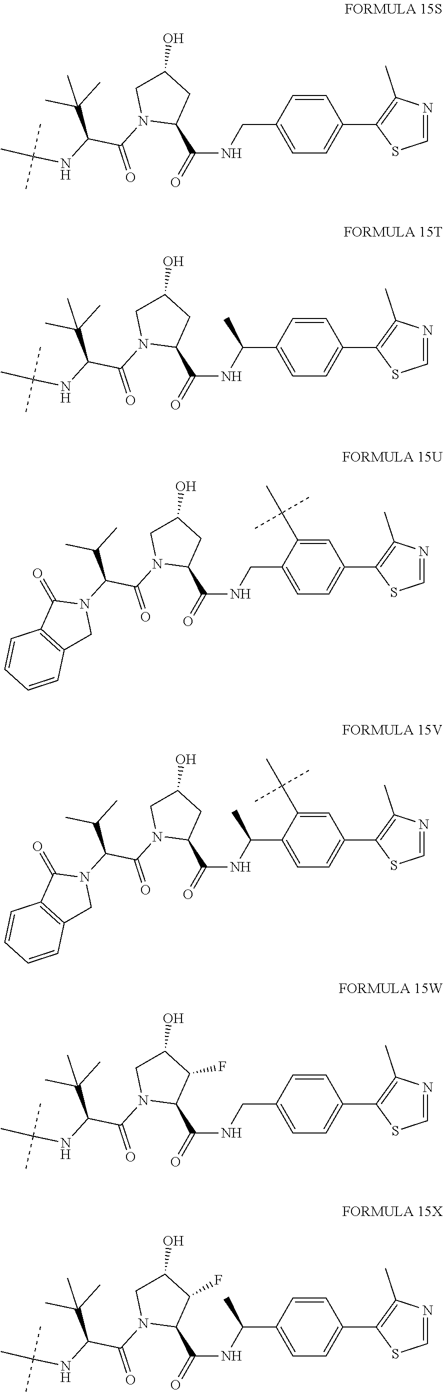

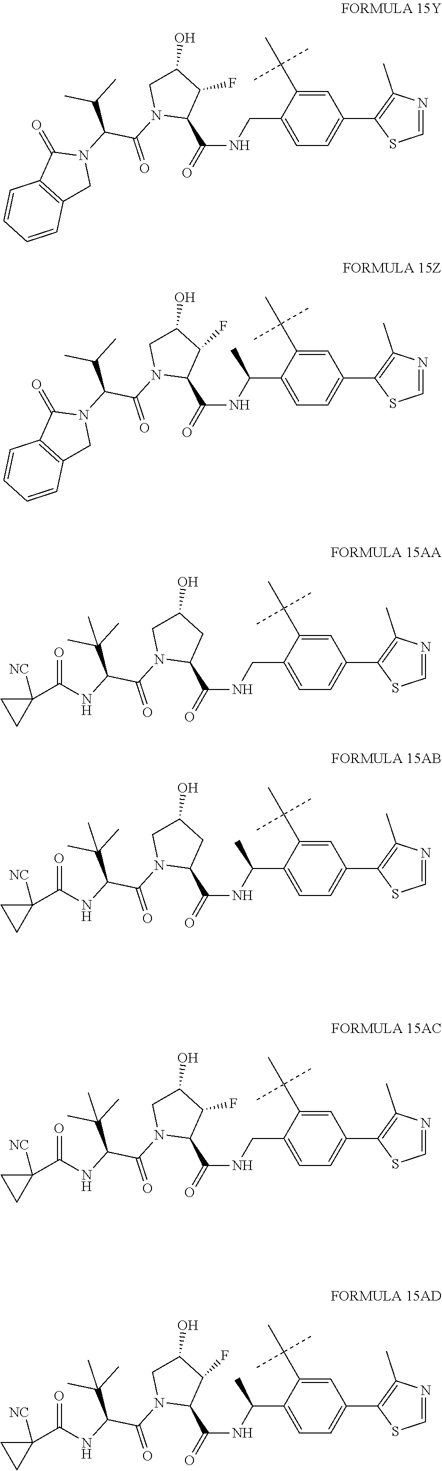

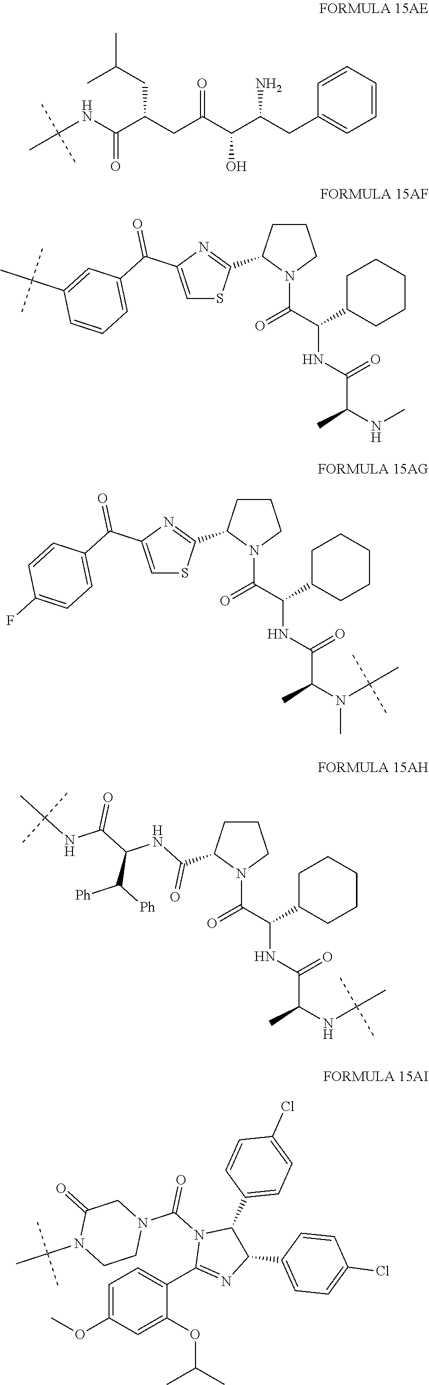

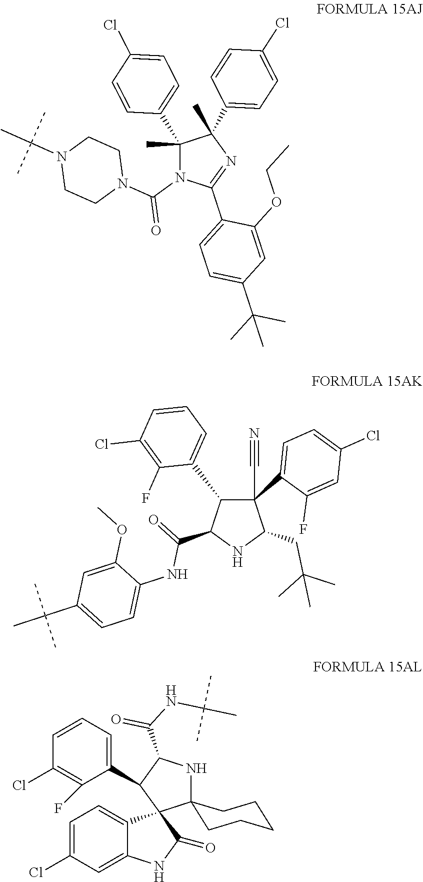

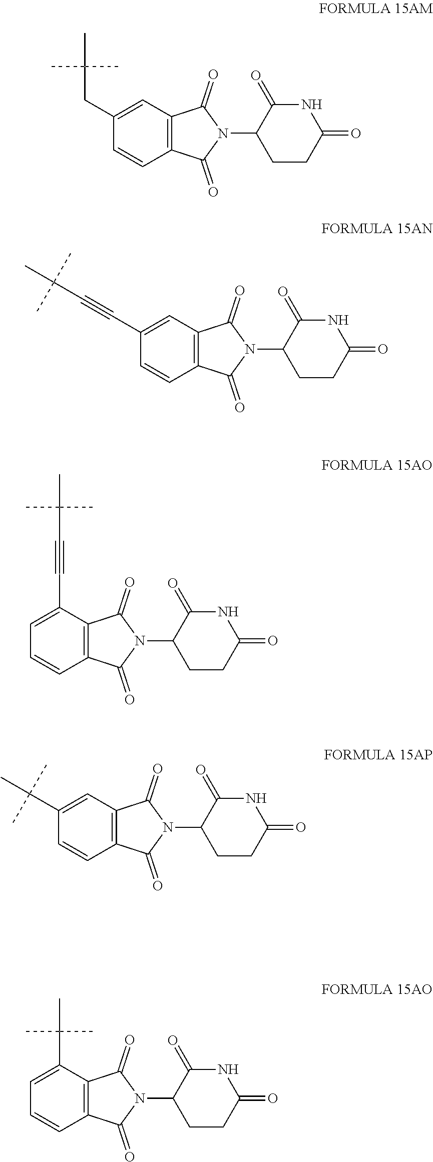

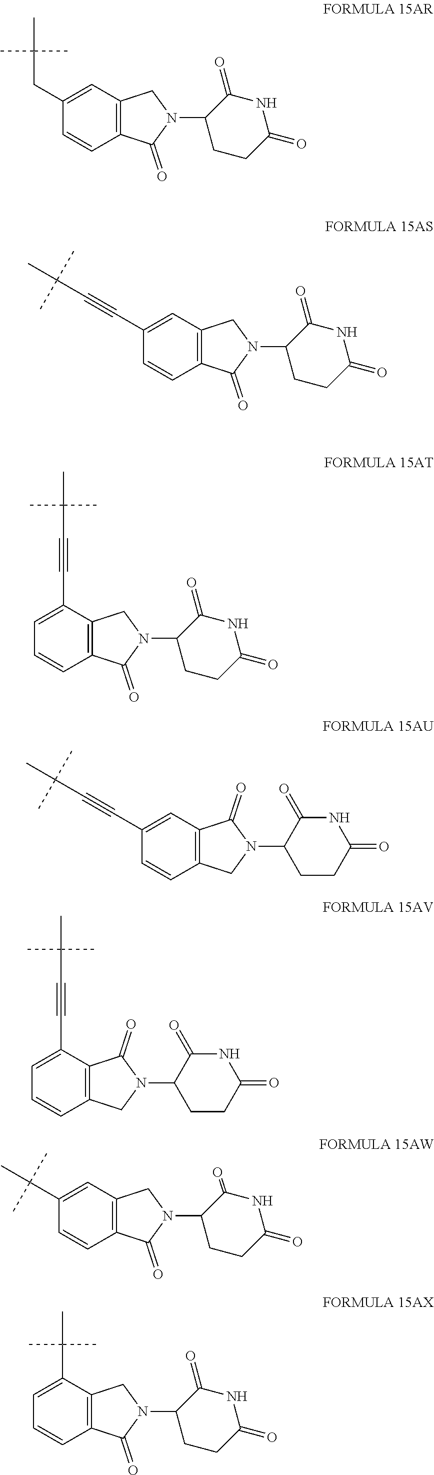

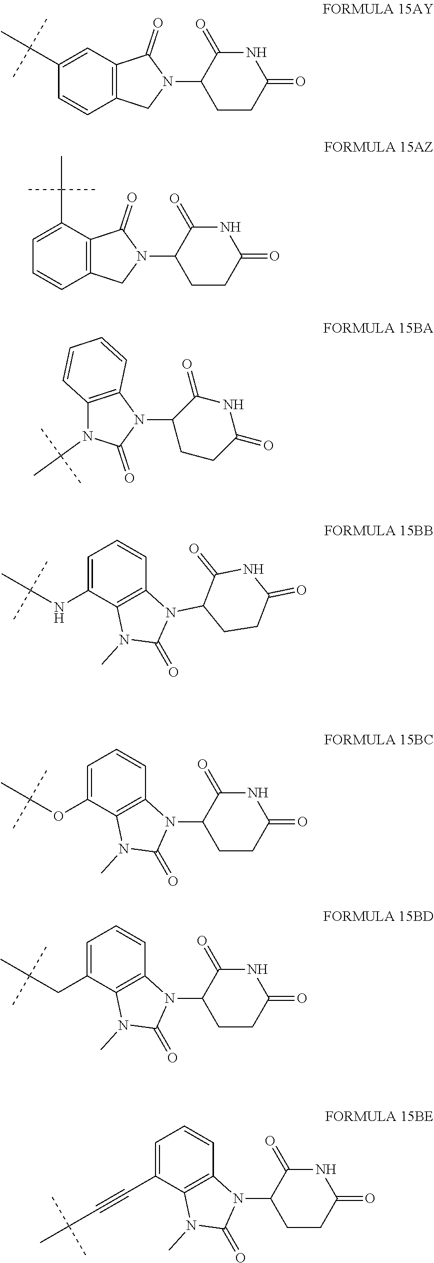

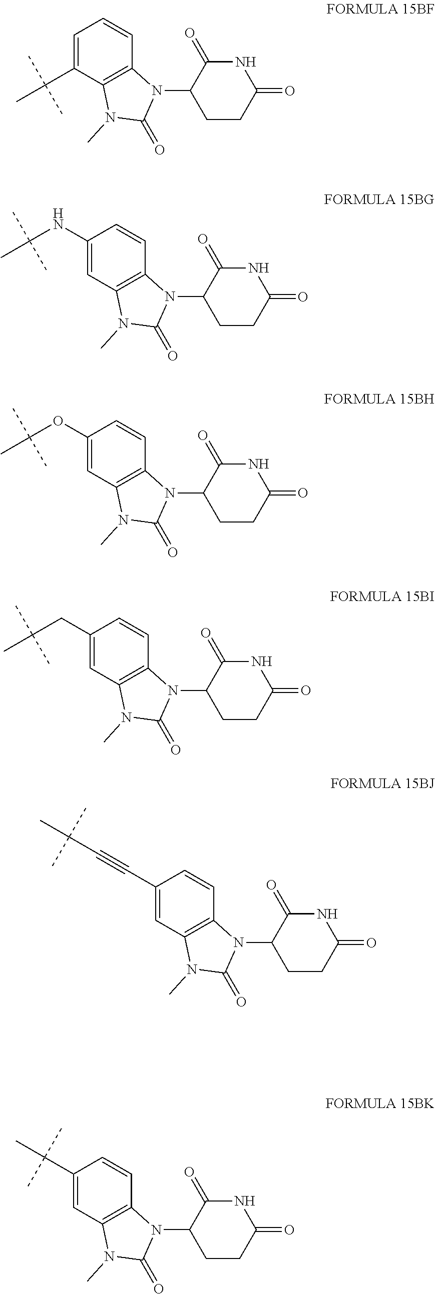

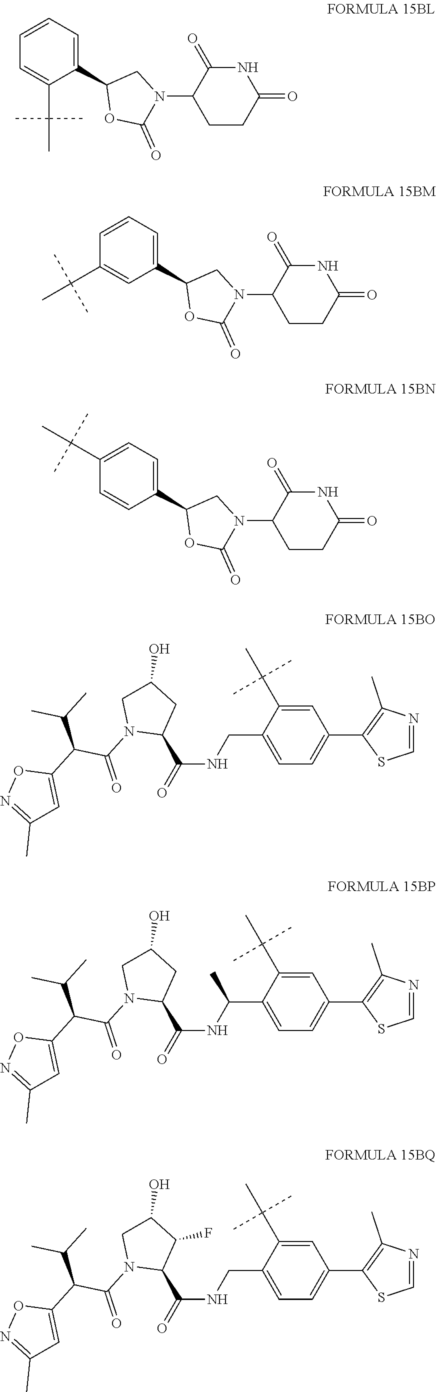

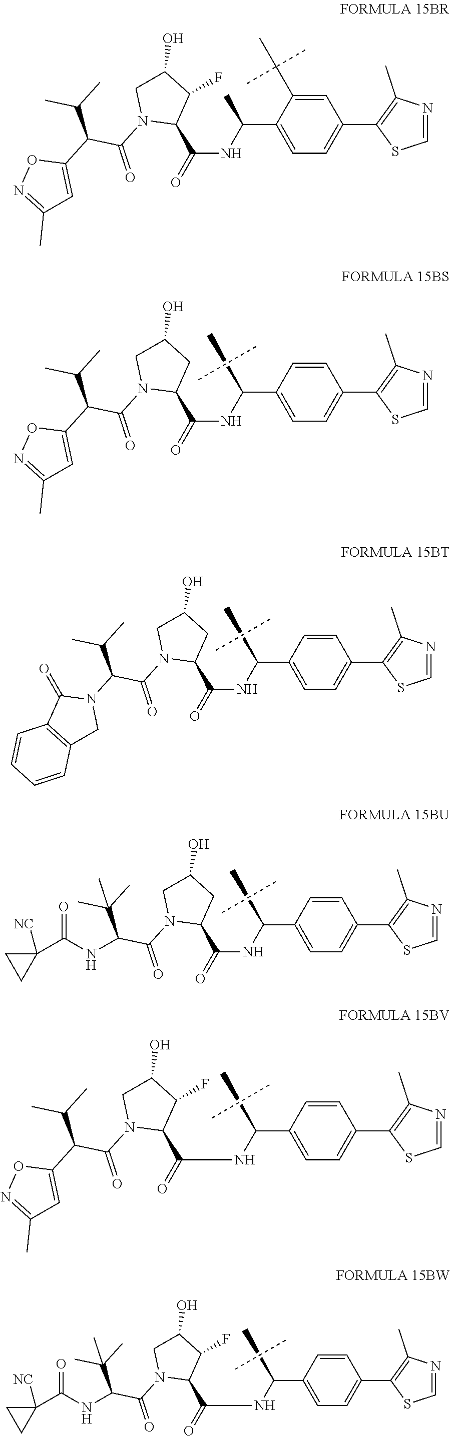

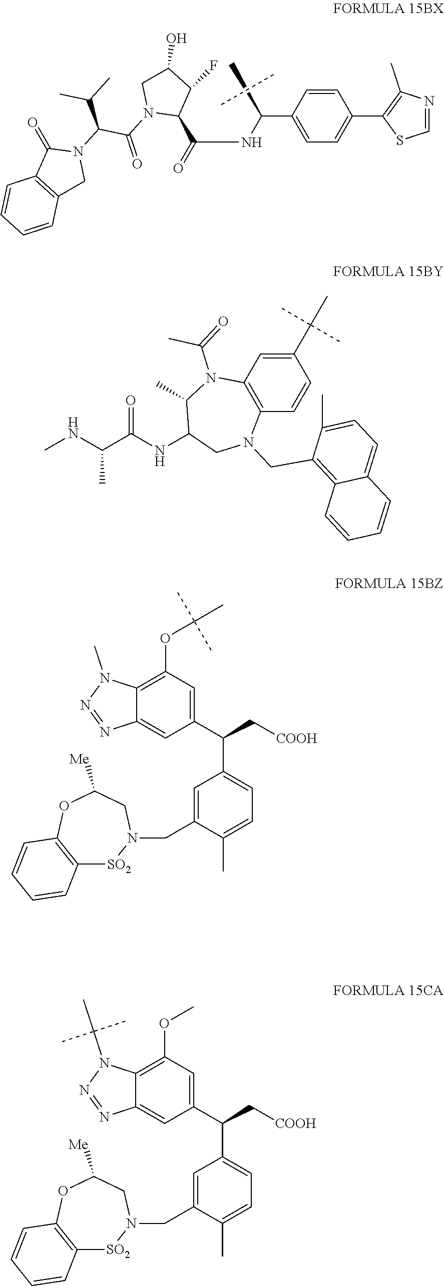

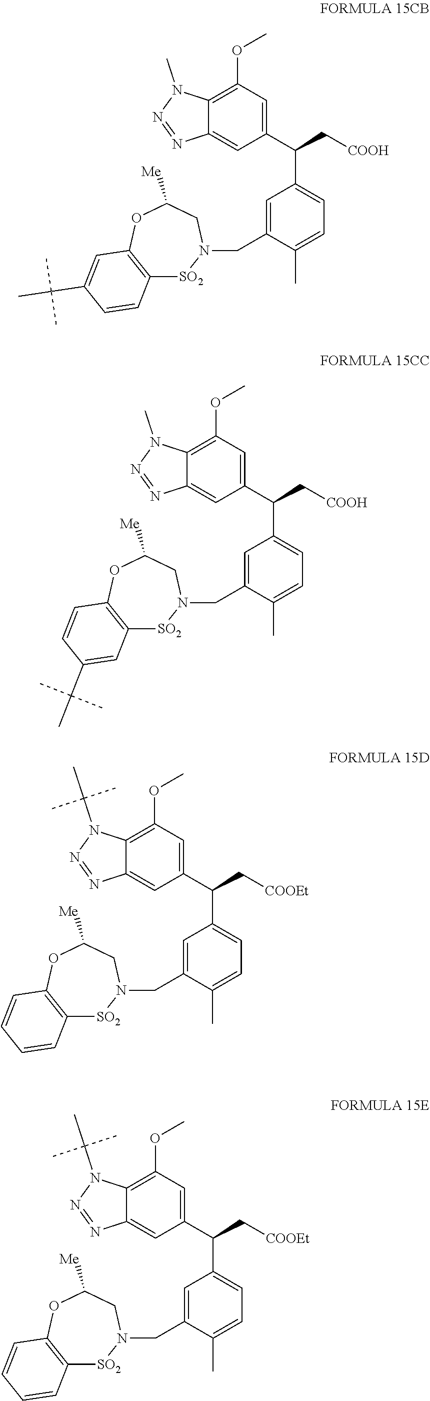

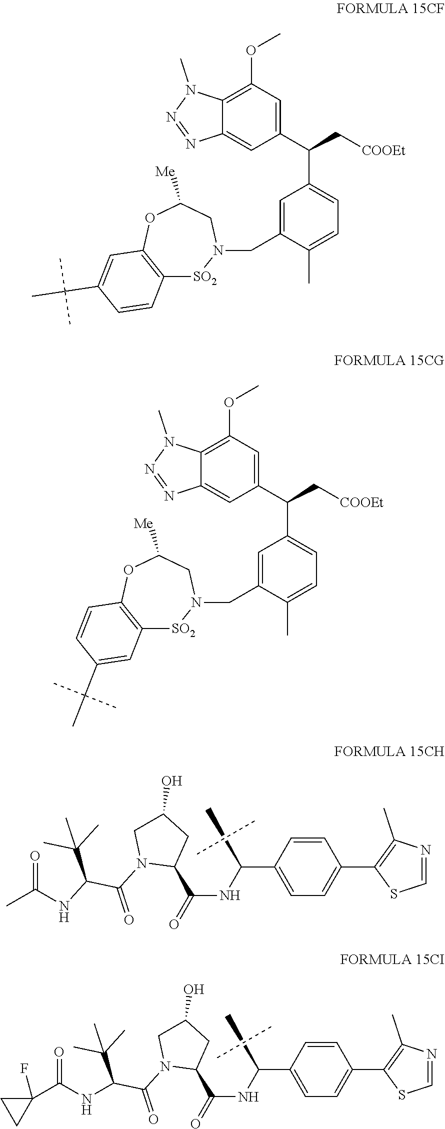

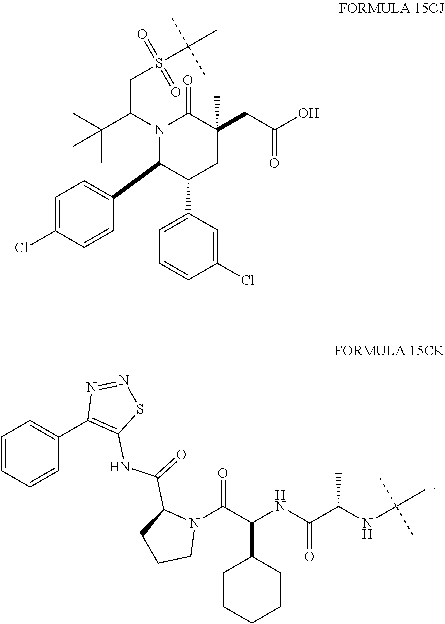

20. The bivalent compound of claim 19, wherein the degradation/disruption tag is one of FORMULAE 15A-15R.

21. The bivalent compound of claim 19, wherein the degradation/disruption tag is one of FORMULAE 15AM-15BN.

22. The bivalent compound of claim 1, wherein the linker is selected from the group consisting of:
a moiety according to FORMULA 16: 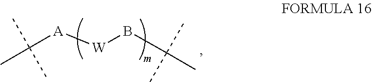
wherein
A, W, and B, at each occurrence, are independently selected from null, CO, CO₂, C(O)NR¹, C(S)NR¹, O, S, SO, SO₂, SO₂NR¹, NR¹, NR¹CO, NR¹CONR², NR⁷C(S), optionally substituted C₁-C₈ alkyl, optionally substituted C₁-C₈ alkoxy, optionally substituted C₁-C₈alkoxyC₁-C₈alkyl, optionally substituted C₁-C₈ haloalkyl, optionally substituted C₁-C₈ hydroxyalkyl, optionally substituted C₂-C₈ alkenyl, optionally substituted C₂-C₈ alkynyl, optionally substituted 3-8 membered cycloalkyl, optionally substituted C₃-C₈ cycloalkoxy, optionally substituted 3-8 membered heterocyclyl, optionally substituted aryl, optionally substituted heteroaryl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, and optionally substituted C₃-C₁₃ spiro heterocyclyl; wherein
R¹ and R² are independently selected from hydrogen, optionally substituted C₁-C₈ alkyl, optionally substituted C₃-C₈ cycloalkyl, optionally substituted 3-8 membered cycloalkoxy, optionally substituted 3-8 membered heterocyclyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, and optionally substituted C₁-C₈alkylaminoC₁-C₈alkyl; and
m is 0 to 15;
a moiety according to FORMULA 16A: 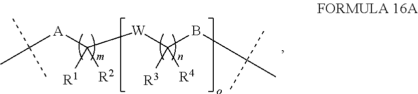
wherein
R¹, R², R³, and R⁴, at each occurrence, are independently selected from hydrogen, halogen, CN, OH, NH₂, optionally substituted C₁-C₈ alkyl, optionally substituted C₃-C₈ cycloalkyl, optionally substituted C₃-C₈ cycloalkoxy, optionally substituted 3-8 membered heterocyclyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, and optionally substituted C₁-C₈ alkylaminoC₁-C₈ alkyl;
A, W, and B, at each occurrence, are independently selected from null, CO, CO₂, C(O)NR⁵, C(S)NR⁵, O, S, SO, SO₂, SO₂NR⁵, NR⁵, NR⁵CO, NR⁵CONR⁶, NR⁵C(S), optionally substituted C₁-C₈ alkyl, optionally substituted C₁-C₈ alkoxy, optionally substituted C₁-C₈alkoxyC₁-C₈alkyl, optionally substituted C₁-C₈ haloalkyl, optionally substituted C₁-C₈ hydroxyalkyl, optionally substituted C₂-C₈ alkenyl, optionally substituted C₂-C₈ alkynyl, optionally substituted 3-8 membered cycloalkyl, optionally substituted C₃-C₈ cycloalkoxy, optionally substituted 3-8 membered heterocyclyl, optionally substituted aryl, optionally substituted heteroaryl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, and optionally substituted C₃-C₁₃ spiro heterocyclyl; wherein
R⁵ and R⁶ are independently selected from hydrogen, optionally substituted C₁-C₈ alkyl, optionally substituted 3-8 membered cycloalkyl, optionally substituted C₃-C₈ cycloalkoxy, optionally substituted 3-8 membered heterocyclyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, and optionally substituted C₁-C₈alkylaminoC₁-C₈alkyl;
m is 0 to 15;
n, at each occurrence, is 0 to 15; and
is 0 to 15;
a moiety according to FORMULA 16B: 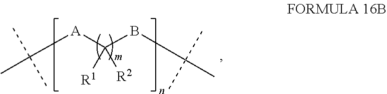
wherein
R¹ and R², at each occurrence, are independently selected from hydrogen, halogen, CN, OH, NH₂, and optionally substituted C₁-C₈ alkyl, optionally substituted 3-8 membered cycloalkyl, optionally substituted C₃-C₈ cycloalkoxy, optionally substituted 3-8 membered heterocyclyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, or C₁-C₈alkylaminoC₁-C₈alkyl;
A and B, at each occurrence, are independently selected from null, CO, CO₂, C(O)NR³, C(S)NR³, O, S, SO, SO₂, SO₂NR³, NR³, NR³CO, NR³CONR⁴, NR³C(S), and optionally substituted C₁-C₈ alkyl, optionally substituted C₁-C₈ alkoxy, optionally substituted C₁-C₈alkoxyC₁-C₈alkyl, optionally substituted C₁-C₈ haloalkyl, optionally substituted C₁-C₈ hydroxyalkyl, optionally substituted C₂-C₈ alkenyl, optionally substituted C₂-C₈ alkynyl, optionally substituted 3-8 membered cycloalkyl, optionally substituted C₃-C₈ cycloalkoxy, optionally substituted 3-8 membered heterocyclyl, optionally substituted aryl, optionally substituted heteroaryl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, or C₃-C₁₃ spiro heterocyclyl; wherein
R³ and R⁴ are independently selected from hydrogen, and optionally substituted C₁-C₈ alkyl, optionally substituted 3-8 membered cycloalkyl, optionally substituted C₃-C₈ cycloalkoxy, optionally substituted 3-8 membered heterocyclyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, or C₁-C₈alkylaminoC₁-C₈alkyl;
each m is 0 to 15; and
n is 0 to 15;
a moiety according to FORMULA 16C: 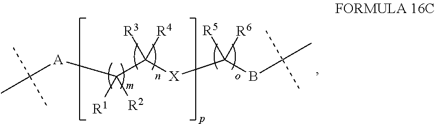
wherein
X is selected from O, NH, and NR⁷;
R¹, R², R³, R⁴, R⁵, and R⁶, at each occurrence, are independently selected from hydrogen, halogen, CN, OH, NH₂, optionally substituted C₁-C₈ alkyl, optionally substituted 3-8 membered cycloalkyl, optionally substituted C₃-C₈ cycloalkoxy, optionally substituted 3-8 membered heterocyclyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, and optionally substituted C₁-C₈ alkylaminoC₁-C₈ alkyl;
A and B, at each occurrence, are independently selected from null, CO, NH, NH—CO, CO—NH, CH₂—NH—CO, CH₂—CO—NH, NH—CO—CH₂, CO—NH—CH₂, CH₂—NH—CH₂—CO—NH, CH₂—NH—CH₂—NH—CO, —CO—NH, CO—NH—CH₂—NH—CH₂, CH₂—NH—CH₂, CO₂, C(O)NR⁷, C(S)NR⁷, O, S, SO, SO₂, SO₂NR⁷, NR⁷, NR⁷CO, NR⁷CONR⁸, NR⁷C(S), optionally substituted C₁-C₈ alkyl, optionally substituted C₁-C₈ alkoxy, optionally substituted C₁-C₈alkoxyC₁-C₈alkyl, optionally substituted C₁-C₈ haloalkyl, optionally substituted C₁-C₈ hydroxyalkyl, optionally substituted C₂-C₈ alkenyl, optionally substituted C₂-C₈ alkynyl, optionally substituted 3-8 membered cycloalkyl, optionally substituted C₃-C₈ cycloalkoxy, optionally substituted 3-8 membered heterocyclyl, optionally substituted aryl, optionally substituted heteroaryl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, and optionally substituted C₃-C₁₃ spiro heterocyclyl; wherein
R⁷ and R⁸ are independently selected from hydrogen, optionally substituted C₁-C₈ alkyl, optionally substituted 3-8 membered cycloalkyl, optionally substituted C₃-C₈ cycloalkoxy, optionally substituted 3-8 membered heterocyclyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, and optionally substituted C₁-C₈ alkylaminoC₁-C₈ alkyl;
m, at each occurrence, is 0 to 15;
n, at each occurrence, is 0 to 15;
o is 0 to 15;
p is 0 to 15; and
a moiety according to one of FORMULAE C1, C2, C3, C4 and C8:

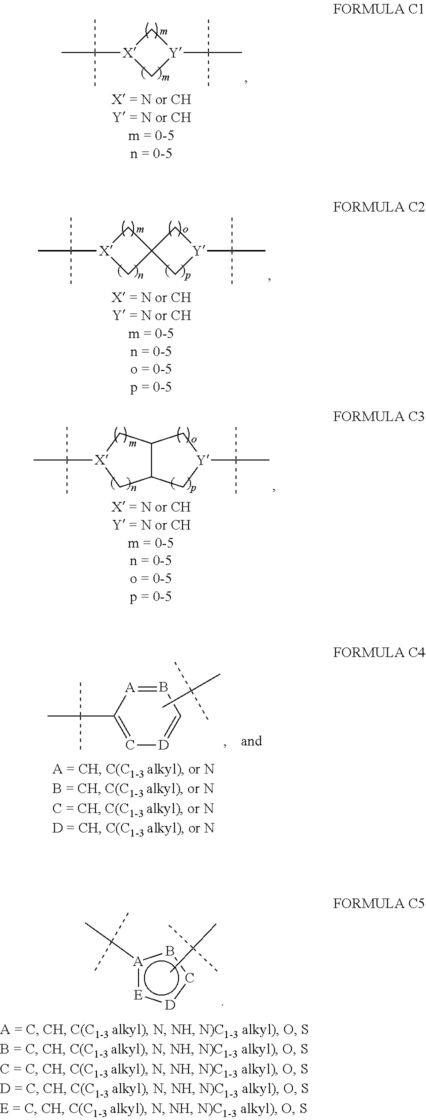

23. The bivalent compound of claim 22, wherein the linker is the moiety of FORMULA 16.

24. The bivalent compound of claim 22, wherein the linker is the moiety of FORMULA 16A.

25. The bivalent compound of claim 22, wherein the linker is the moiety of FORMULA 16B.

26. The bivalent compound of claim 22, wherein the linker is the moiety of FORMULA 16C

27. The bivalent compound of claim 22, wherein the linker is the moiety of one of FORMULAE C1-C8.

28. The bivalent compound of claim 1, wherein the linker is selected from the group consisting of a ring selected from the group consisting of a 3 to 13 membered ring; a 3 to 13 membered fused ring; a 3 to 13 membered bridged ring; and a 3 to 13 membered spiro ring.

29. A bivalent compound selected from the group consisting of:
MS-5-006, MS-5-007, MS-5-008, MS-5-009, MS-5-010, MS-5-011, MS-5-015, MS-5-016, MS-5-017, MS-5-018, MS-5-019, MS-5-021, MS-5-022, MS-5-024, MS-5-026, MS-5-027, MS-5-028, MS-5-029, MS-5-030, MS-5-033, MS-5-034, MS-5-035, MS-5-036, MS-5-037, MS-5-038, MS-5-039, MS-5-040, MS-5-041, MS-5-042, MS-5-043, MS-5-044, MS-5-045, MS-5-046, MS-6-003, MS-6-004, MS-6-005, MS-6-006, MS-6-007, MS-6-008, MS-6-009, MS-6-013, MS-6-014, MS-6-015, MS-6-016, MS-6-017, MS-6-018, MS-6-019, MS-6-020, MS-6-021, MS-6-022, MS-6-023, MS-6-024, MS-6-025, MS-6-026, MS-6-027, MS-6-028, MS-6-029, MS-6-037, MS-6-038, MS-6-039, MS-6-040, MS-6-041, MS-6-043, MS-6-044, MS-6-045, MS-6-046, MS-6-047, MS-6-048, MS-6-049, MS-6-050, MS-6-051, MS-6-052, MS-6-053, MS-6-054, MS-6-055, MS-6-058, MS-6-059, MS-6-060 and MS-6-061.

30. A bivalent compound selected from the group consisting of:
MS-5-006, MS-5-007, MS-5-008, MS-5-009, MS-5-010, MS-5-011, MS-5-015, MS-5-016, MS-5-017, MS-5-018, MS-5-019, MS-5-021, MS-5-022, MS-5-024, MS-5-026, MS-5-027, MS-5-028, MS-5-029, MS-5-030, MS-5-033, MS-5-034, MS-5-035, MS-5-036, MS-5-037, MS-5-038, MS-5-039, MS-5-040, MS-5-041, MS-5-042, MS-5-043, MS-5-044, MS-5-045 and MS-5-046.

31. A bivalent compound selected from the group consisting of:
MS-6-003, MS-6-004, MS-6-005, MS-6-006, MS-6-007, MS-6-008, MS-6-009, MS-6-013, MS-6-014, MS-6-015, MS-6-016, MS-6-017, MS-6-018, MS-6-019, MS-6-020, MS-6-021, MS-6-022, MS-6-023, MS-6-024, MS-6-025, MS-6-026, MS-6-027, MS-6-028, MS-6-029, MS-6-037, MS-6-038, MS-6-039, MS-6-040, MS-6-041, MS-6-043, MS-6-044, MS-6-045, MS-6-046, MS-6-047, MS-6-048, MS-6-049, MS-6-050, MS-6-051, MS-6-052, MS-6-053, MS-6-054, MS-6-055, MS-6-058, MS-6-059, MS-6-060 and MS-6-061.

32. A bivalent compound selected from the group consisting of:
a) 2-(4-(3-(5-(2-((3,3-dimethyl-1-oxo-1,3-dihydroisobenzofuran-5-yl)amino)-4-(((S)-2-hydroxy-1-phenylethyl)amino)pyrimidin-5-yl)-1,3,4-oxadiazol-2-yl)propyl)piperazin-1-yl)-N-(5-((2-(2,6-dioxopiperidin-3-yl)-1,3-dioxoisoindolin-4-yl)amino)pentyl)acetamide (MS-5-007);
b) 2-(4-(3-(5-(2-((3,3-dimethyl-1-oxo-1,3-dihydroisobenzofuran-5-yl)amino)-4-(((S)-2-hydroxy-1-phenylethyl)amino)pyrimidin-5-yl)-1,3,4-oxadiazol-2-yl)propyl)piperazin-1-yl)-N-(8-((2-(2,6-dioxopiperidin-3-yl)-1,3-dioxoisoindolin-4-yl)amino)octyl)acetamide (MS-5-008);
c) 4-((5-(5-(2-((3,3-dimethyl-1-oxo-1,3-dihydroisobenzofuran-5-yl)amino)-4-(((S)-2-hydroxy-1-phenylethyl)amino)pyrimidin-5-yl)-1,3,4-oxadiazol-2-yl)pentyl)amino)-2-(2,6-dioxopiperidin-3-yl)isoindoline-1,3-dione (MS-5-009);
d) 4-((6-(5-(2-((3,3-dimethyl-1-oxo-1,3-dihydroisobenzofuran-5-yl)amino)-4-(((S)-2-hydroxy-1-phenylethyl)amino)pyrimidin-5-yl)-1,3,4-oxadiazol-2-yl)hexyl)amino)-2-(2,6-dioxopiperidin-3-yl)isoindoline-1,3-dione (5MS-5-010); and
e) 4-((8-(5-(2-((3,3-dimethyl-1-oxo-1,3-dihydroisobenzofuran-5-yl)amino)-4-(((S)-2-hydroxy-1-phenylethyl)amino)pyrimidin-5-yl)-1,3,4-oxadiazol-2-yl)octyl)amino)-2-(2,6-dioxopiperidin-3-yl)isoindoline-1,3-dione (MS-5-011).

33. A bivalent compound selected from the group consisting of:
f) 2-(2,6-dioxopiperidin-3-yl)-4-((3-(((2-(6-(4-ethyl-4H-1,2,4-triazol-3-yl)pyridin-2-yl)-6-((R)-2-methylpyrrolidin-1-yl)-1-oxo-2,3-dihydro-1H-pyrrolo[3,4-c]pyridin-4-yl)methyl)(methyl)amino)propyl)amino)isoindoline-1,3-dione (MS-6-007);
g) 2-(2,6-dioxopiperidin-3-yl)-4-((4-(((2-(6-(4-ethyl-4H-1,2,4-triazol-3-yl)pyridin-2-yl)-6-((R)-2-methylpyrrolidin-1-yl)-1-oxo-2,3-dihydro-1H-pyrrolo[3,4-c]pyridin-4-yl)methyl)(methyl)amino)butyl)amino)isoindoline-1,3-dione (MS-6-008);
h) 2-(2,6-dioxopiperidin-3-yl)-4-((6-(((2-(6-(4-ethyl-4H-1,2,4-triazol-3-yl)pyridin-2-yl)-6-((R)-2-methylpyrrolidin-1-yl)-1-oxo-2,3-dihydro-1H-pyrrolo[3,4-c]pyridin-4-yl)methyl)(methyl)amino)hexyl)amino)isoindoline-1,3-dione (MS-6-009); and
i) 2-((2-(2,6-dioxopiperidin-3-yl)-1,3-dioxoisoindolin-4-yl)amino)-N-((2-(6-(4-ethyl-4H-1,2,4-triazol-3-yl)pyridin-2-yl)-6-((R)-2-methylpyrrolidin-1-yl)-1-oxo-2,3-dihydro-1H-pyrrolo[3,4-c]pyridin-4-yl)methyl)-N-methylacetamide (MS-6-024).

34. A pharmaceutical composition comprising:
a bivalent compound according to one of claims 1-33; and
a pharmaceutically acceptable carrier.

35. A method of treating an HPK1-mediated disease, comprising administering to a subject in need thereof a pharmaceutical composition according to claim 34.

36. A method according to claim 35, wherein the HPK1-mediated disease is cancer.

37. The bivalent compound of claim 1, wherein the HPK1 ligand is the moiety according to FORMULA 29.
